(19) United States
(12) Patent Application Publication
(10) Pub. No.: US 2025/0066455 A1
(43) Pub. Date: Feb. 27, 2025

(54) NEUTRALISING ANTIBODIES AND USES THEREOF

(71) Applicant: The University of Melbourne, Melbourne (AU)

(72) Inventors: Adam Kenneth Wheatley, Melbourne (AU); Hyon Tan, Melbourne (AU); Stephen John Kent, Melbourne (AU)

(21) Appl. No.: 18/293,451

(22) PCT Filed: Jul. 29, 2022
(86) PCT No.: PCT/AU2022/050818

§ 371 Date: Jan. 30, 2024

(30) Foreign Application Priority Data
Jul. 30, 2021 (AU) 2021902365
Mar. 23, 2022 (AU) 2022900732

(51) Int. Cl. C07K 16/10; A61K 39/00; A61P 31/14

(52) U.S. Cl. CPC C07K 16/1003; A61P 31/14; A61K 2039/505; C07K 2317/55; C07K 2317/76; C07K 2317/92

(57) ABSTRACT

The present invention relates to antibodies or antigen-binding fragments thereof that neutralise SARS-CoV-2, and uses thereof.

### TECHNICAL FIELD

The present invention relates to antibodies or antigen-binding fragments thereof that neutralise SARS-CoV-2, and uses thereof.

### BACKGROUND OF INVENTION

The global spread of SARS-CoV-2 has sparked intense global research efforts to combat the significant health and economic impacts of the pandemic. Monoclonal antibodies (mAbs) have demonstrated promise as treatments or prophylactic agents against other viral diseases such as RSV (Rocca et al., 2021) and Ebola (Misasi and Sullivan, 2021).

The clinical development of SARS-CoV-2 antibody-based therapeutics (reviewed in Taylor et al., 2021) has seen rapid progression of candidate mAbs through clinical trials, with two human mAb cocktails (casirivimab/imdevimab and bamlanivimab/etesevimab) and one monotherapy (bamlanivimab) conditionally approved for treatment of high-risk ambulatory patients. However, some of these first-generation treatments suffer significant losses of neutralisation potency in the face of ongoing viral evolution, with near complete loss of activity against B.1.351 and P.1 SARS-CoV-2 variants of concern (VOC) reported for many neutralising human mAbs (Wang et al., 2021 b; Wang et al., 2021c; Wang et al., 2021d; Zhou et al., 2021).

There is a need for alternative antibodies that have SARS-CoV-2 neutralising activity. There is also a need for antibodies that have SARS-CoV-2 neutralising activity against Omicron Variants of Concern.

There is also a need to accurately identify and characterise antibody epitopes across the critical viral spike protein which consistently yield robust in vitro and in vivo neutralisation outcomes.

### SUMMARY OF INVENTION

In one embodiment the present invention provides an antibody or an antigen-binding fragment thereof that neutralises SARS-CoV-2.

In one embodiment the present invention provides an antibody or an antigen-binding fragment thereof that neutralises a SARS-CoV-2 variant selected from the group consisting of B.1.1.7 (Alpha), B.1.351 (Beta), B.1.617.2 (Delta), P.1 (Gamma), B.1.427 (Epsilon) B.1.429 (Epsilon), B.1.525 (Eta), B.1.526 (Iota) B.1.617.1 (Kappa), B.1.617.3, and B.1.1.529 (Omicron).

In one embodiment the present invention provides an antibody or an antigen-binding fragment thereof as described herein, wherein the antibody or an antigen-binding fragment thereof that neutralises SARS-CoV-2 with an IC50 of less than 2 ug/ml.

In one embodiment the present invention provides an antibody or an antigen-binding fragment thereof as described herein wherein the antibody is not REGN10933 and REGN10987.

In one embodiment the present invention provides an antibody or an antigen-binding fragment thereof as described herein that binds to epitope cluster 1 of the SARS-CoV-2 spike receptor-binding domain (RBD).

In one embodiment the present invention provides an antibody or an antigen-binding fragment thereof as described herein that binds to epitope cluster 2 of the SARS-CoV-2 spike RBD.

In one embodiment the present invention provides an antibody or an antigen-binding fragment thereof as described herein that binds to epitope cluster 3 of the SARS-CoV-2 spike RBD.

In one embodiment the present invention provides an antibody or an antigen-binding fragment thereof as described herein that binds to epitope cluster 4 of the SARS-CoV-2 spike RBD.

In one embodiment the present invention provides an antibody or an antigen-binding fragment thereof as described herein that binds to epitope cluster 5 of the SARS-CoV-2 spike RBD.

In one embodiment the present invention provides an antibody or an antigen-binding fragment thereof as described herein that binds to epitope cluster 6 of the SARS-CoV-2 spike RBD.

In one embodiment the present invention provides an antibody or an antigen-binding fragment thereof as described herein wherein the antibody or an antigen-binding fragment thereof comprises a HCDR1 of EFIVSRNY (SEQ ID NO: 18), a HCDR2 of IYSGGTT (SEQ ID NO: 44), a HCDR3 of ARDRGDYLFDY (SEQ ID NO: 70), a LCDR1 of QSISSW (SEQ ID NO: 96), a LCDR2 of KAS (SEQ ID NO: 122), and a LCDR3 of QQYNSYFPT (SEQ ID NO: 148).

In one embodiment the present invention provides an antibody or an antigen-binding fragment thereof as described herein wherein the antibody or an antigen-binding fragment thereof comprises a HCDR1 of GLTVSSNY (SEQ ID NO: 20), a HCDR2 of FYPGGST (SEQ ID NO: 46), a HCDR3 of ARDAVYYGMDV (SEQ ID NO: 72), a LCDR1 of QSISSY (SEQ ID NO: 98), a LCDR2 of AAS (SEQ ID NO: 124), and a LCDR3 of QESYSTPGLFT (SEQ ID NO: 150).

In one embodiment the present invention provides an antibody or an antigen-binding fragment thereof as described herein wherein the antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134).

In one embodiment the present invention provides an antibody or an antigen-binding fragment thereof as described herein wherein the antibody or an antigen-binding fragment thereof comprises a HCDR1 of GLTVSSNY (SEQ ID NO: 19), a HCDR2 of IYSGGST (SEQ ID NO: 45), a HCDR3 of ARVGGYCSSANCVSDV (SEQ ID NO: 71), a LCDR1 of QDIRNY (SEQ ID NO: 97), a LCDR2 of DAS (SEQ ID NO: 123), and a LCDR3 of QQYDNLPIT (SEQ ID NO: 149).

In one embodiment the present invention provides an antibody or an antigen-binding fragment thereof as described herein wherein the antibody or an antigen-binding fragment thereof comprises a HCDR1 of GETLSGYY (SEQ ID NO: 11), a HCDR2 of ISLRGTA (SEQ ID NO: 37), a HCDR3 of VGGVVLDNVVWFDP (SEQ ID NO: 63), a LCDR1 of QSVGTY (SEQ ID NO: 89), a LCDR2 of NAS (SEQ ID NO: 115), and a LCDR3 of QQRSNWLT (SEQ ID NO: 141).

In one embodiment the present invention provides an antibody or an antigen-binding fragment thereof as described herein wherein the antibody or an antigen-binding fragment thereof comprises a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154).

In one embodiment the present invention provides an antibody or an antigen-binding fragment thereof as described herein wherein the antibody or an antigen-binding fragment thereof comprises a HCDR1 of GFTFSDYY (SEQ ID NO: 24), a HCDR2 of ISGGSSFT (SEQ ID NO: 50), and a HCDR3 of ARSPPTGDSSDWYDSPAYNNYYMDV (SEQ ID NO: 76).

In one embodiment the present invention provides an antibody or an antigen-binding fragment thereof as described herein wherein the antibody or an antigen-binding fragment thereof comprises a HCDR1 of GFTFSYAW (SEQ ID NO: 21), a HCDR2 of IKRKSDGGTT (SEQ ID NO: 47), a HCDR3 of TTDLCRSTSCEHDAFDI (SEQ ID NO: 73), a LCDR1 of QSIRSY (SEQ ID NO: 99), a LCDR2 of AAS (SEQ ID NO: 125), and a LCDR3 of QQSYTTPAIT (SEQ ID NO: 151).

In one embodiment the present invention provides an antibody or an antigen-binding fragment thereof as described herein wherein the antibody or an antigen-binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133).

In one embodiment the present invention provides an antibody or an antigen-binding fragment thereof as described herein wherein the antibody or an antigen-binding fragment thereof comprises a HCDR1 of GFNFYYSA (SEQ ID NO: 1), a HCDR2 of IVVGSGNT (SEQ ID NO: 27), a HCDR3 of AAPYCSGGSCHDGFDI (SEQ ID NO: 53), a LCDR1 of QSVRSNY (SEQ ID NO: 79), a LCDR2 of GAS (SEQ ID NO: 105), and a LCDR3 of QQYGSSPWT (SEQ ID NO: 131).

In one embodiment the present invention provides an antibody or an antigen-binding fragment thereof as described herein wherein the antibody or an antigen-binding fragment thereof comprises a HCDR1 of GFTFSNSA (SEQ ID NO: 2), a HCDR2 of IVVGSGNT (SEQ ID NO: 28), a HCDR3 of AAPYCGGDCNDGFDV (SEQ ID NO: 54), a LCDR1 of QSVRSSY (SEQ ID NO: 80), a LCDR2 of STS (SEQ ID NO: 106), and a LCDR3 of QQYGSSPWT (SEQ ID NO: 132).

In one embodiment the present invention provides an antibody or an antigen-binding fragment thereof as described herein wherein the antibody or an antigen-binding fragment thereof comprises a HCDR1 of GFTFDLSA (SEQ ID NO: 5), a HCDR2 of IAVGSGNT (SEQ ID NO: 31), a HCDR3 of AAPYCYSTSCADGFDI (SEQ ID NO: 57), a LCDR1 of QSVRSGY (SEQ ID NO: 83), a LCDR2 of GTS (SEQ ID NO: 109), and a LCDR3 of QQYGSSPWT (SEQ ID NO: 135).

In one embodiment the present invention provides an antibody or an antigen-binding fragment thereof as described herein wherein the antibody or an antigen-binding fragment thereof comprises a HCDR1 of GFTFSNAW (SEQ ID NO: 6), a HCDR2 of IKSKTDGGTT (SEQ ID NO: 32), a HCDR3 of TTDPGWWRIAVAGTNY (SEQ ID NO: 58), a LCDR1 of QSISSY (SEQ ID NO: 84), a LCDR2 of AAS (SEQ ID NO: 110), and a LCDR3 of QQSDSSPPT (SEQ ID NO: 136).

In one embodiment the present invention provides an antibody or an antigen-binding fragment thereof as described herein wherein the antibody or an antigen-binding fragment thereof comprises a HCDR1 of GFTFTLSA (SEQ ID NO: 7), a HCDR2 of IVPGSGNV (SEQ ID NO: 33), a HCDR3 of AAPYCNKTRCSDGFDI (SEQ ID NO: 59), a LCDR1 of QSVSSSY (SEQ ID NO: 85), a LCDR2 of GAS (SEQ ID NO: 111), and a LCDR3 of QQYGSSLFT (SEQ ID NO: 137).

In one embodiment the present invention provides an antibody or an antigen-binding fragment thereof as described herein wherein the antibody or an antigen-binding fragment thereof comprises a HCDR1 of GFTFTSSA (SEQ ID NO: 8), a HCDR2 of IVVGSGNT (SEQ ID NO: 34), a HCDR3 of AAPNCNRTICADGFDI (SEQ ID NO: 60), a LCDR1 of QSVRSSY (SEQ ID NO: 86), a LCDR2 of ATS (SEQ ID NO: 112), and a LCDR3 of QQYGSSPWT (SEQ ID NO: 138).

In one embodiment the present invention provides an antibody or an antigen-binding fragment thereof as described herein wherein the antibody or an antigen-binding fragment thereof comprises a HCDR1 of GFTFTTSA (SEQ ID NO: 9), a HCDR2 of IAVGSGNT (SEQ ID NO: 35), a HCDR3 of AAPYCNTTSCDDGFDI (SEQ ID NO: 61), a LCDR1 of QSVRSNY (SEQ ID NO: 87), a LCDR2 of GAS (SEQ ID NO: 113), and a LCDR3 of QQYGSSPWT (SEQ ID NO: 139).

In one embodiment the present invention provides an antibody or an antigen-binding fragment thereof as described herein wherein the antibody or an antigen-binding fragment thereof comprises a HCDR1 of GFIFNRYW (SEQ ID NO: 10), a HCDR2 of IKQDGSEK (SEQ ID NO: 36), a HCDR3 of AADLGILWFGDLRKSEP (SEQ ID NO: 62), a LCDR1 of QGISNS (SEQ ID NO: 88), a LCDR2 of AAS (SEQ ID NO: 114), and a LCDR3 of QEYYSLRT (SEQ ID NO: 140).

In one embodiment the present invention provides an antibody or an antigen-binding fragment thereof as described herein wherein the antibody or an antigen-binding fragment thereof comprises a HCDR1 of GFTYTTSA (SEQ ID NO: 12), a HCDR2 of IVAGSGNT (SEQ ID NO: 38), a HCDR3 of AAPGCNTTICPDGFDI (SEQ ID NO: 64), a LCDR1 of QSVSSSY (SEQ ID NO: 90), a LCDR2 of GAS (SEQ ID NO: 116), and a LCDR3 of QQYGSLPWT (SEQ ID NO: 142).

In one embodiment the present invention provides an antibody or an antigen-binding fragment thereof as described herein wherein the antibody or an antigen-binding fragment thereof comprises a HCDR1 of GGTFSIYA (SEQ ID NO: 13), a HCDR2 of IIPISGTA (SEQ ID NO: 39), a HCDR3 of ARLGRGDYDSSGYYKVYFDY (SEQ ID NO: 65), a LCDR1 of QNIGNY (SEQ ID NO: 91), a LCDR2 of GAS (SEQ ID NO: 117), and a LCDR3 of QKSYSGPYT (SEQ ID NO: 143).

In one embodiment the present invention provides an antibody or an antigen-binding fragment thereof as described herein wherein the antibody or an antigen-binding fragment thereof comprises a HCDR1 of GYRFSNYW (SEQ ID NO: 14), a HCDR2 of IDPSDYYT (SEQ ID NO: 40), a HCDR3 of AKHKFFGELPIRGFDP (SEQ ID NO: 66), a LCDR1 of QSVSGSY (SEQ ID NO: 92), a LCDR2 of GAS (SEQ ID NO: 118), and a LCDR3 of QQYGSSPWT (SEQ ID NO: 144).

In one embodiment the present invention provides an antibody or an antigen-binding fragment thereof as described herein wherein the antibody or an antigen-binding fragment thereof comprises a HCDR1 of GYSFTTHW (SEQ ID NO: 15), a HCDR2 of IDPSDSYT (SEQ ID NO: 41), a HCDR3 of ARENFWSVYYTGIDYYMDV (SEQ ID NO: 67), a LCDR1 of QGISNS (SEQ ID NO: 93), a LCDR2 of AAS (SEQ ID NO: 119), and a LCDR3 of QQYYSTPYT (SEQ ID NO: 145).

In one embodiment the present invention provides an antibody or an antigen-binding fragment thereof as described herein wherein the antibody or an antigen-binding fragment thereof comprises a HCDR1 of GEPLSGYF (SEQ ID NO: 16), a HCDR2 of ISLRGSA (SEQ ID NO: 42), a HCDR3 of SRGVVLNNVVWFDP (SEQ ID NO: 68), a LCDR1 of QSVGSY (SEQ ID NO: 94), a LCDR2 of GVS (SEQ ID NO: 120), and a LCDR3 of QQRSIWLT (SEQ ID NO: 146).

In one embodiment the present invention provides an antibody or an antigen-binding fragment thereof as described herein wherein the antibody or an antigen-binding fragment thereof comprises a HCDR1 of GFTFSTYA (SEQ ID NO: 17), a HCDR2 of ISYDGSTK (SEQ ID NO: 43), a HCDR3 of ARDSEDCSSLSCYLDY (SEQ ID NO: 69), a LCDR1 of QSVLYSSNNKNY (SEQ ID NO: 95), a LCDR2 of WAS (SEQ ID NO: 121), and a LCDR3 of QQYYSTPFT (SEQ ID NO: 147).

In one embodiment the present invention provides an antibody or an antigen-binding fragment thereof as described herein wherein the antibody or an antigen-binding fragment thereof comprises a HCDR1 of GFIVSSNY (SEQ ID NO: 22), a HCDR2 of LYPGGST (SEQ ID NO: 48), a HCDR3 of ARNIYDAFDI (SEQ ID NO: 74), a LCDR1 of QGIGSY (SEQ ID NO: 100), a LCDR2 of AAS (SEQ ID NO: 126), and a LCDR3 of QQLNSYPQGA (SEQ ID NO: 152).

In one embodiment the present invention provides an antibody or an antigen-binding fragment thereof according to any one of claims 1 to 3 wherein the antibody or an antigen-binding fragment thereof comprises a HCDR1 of GFTVSSNY (SEQ ID NO: 25), a HCDR2 of IYSGGST (SEQ ID NO: 51), a HCDR3 of ARLPYGDPA (SEQ ID NO: 77), a LCDR1 of QDIRNY (SEQ ID NO: 103), a LCDR2 of DAS (SEQ ID NO: 129), and a LCDR3 of LQYDNLPLT (SEQ ID NO: 155).

In one embodiment the present invention provides an antibody or an antigen-binding fragment thereof as described herein wherein the antibody or an antigen-binding fragment thereof comprises a HCDR1 of GFTFSSFA (SEQ ID NO: 26), a HCDR2 of ISYDGSTK (SEQ ID NO: 52), a HCDR3 of ARDSEDCSSLSCYLDF (SEQ ID NO: 78), a LCDR1 of QSVLYRSNNKNY (SEQ ID NO: 104), a LCDR2 of WAS (SEQ ID NO: 130), and a LCDR3 of QQYYSTPFT (SEQ ID NO: 156).

In one embodiment the present invention provides an antibody or an antigen-binding fragment thereof according s described herein, wherein the antibody or an antigen-binding fragment thereof neutralises more than one SARS-CoV-2 variant selected from the group consisting of B.1.1.7 (Alpha), B.1.351 (Beta), B.1.617.2 (Delta), P.1 (Gamma), B.1.427 (Epsilon), B.1.429 (Epsilon), B.1.525 (Eta), B.1.526 (Iota) B.1.617.1 (Kappa), B.1.617.3, and B.1.1.529 (Omicron).

In a preferred embodiment, the present invention provides an antibody or an antigen-binding fragment thereof as described herein, wherein the antibody or an antigen-binding fragment thereof neutralises more than one B.1.1.529 (Omicron) variant. For example, Omicron BA.1, Omicron BA.2, and/or Omicron BA.4.

In one embodiment the present invention provides a composition comprising at least one antibody or an antigen-binding fragment thereof as described herein and a pharmaceutically acceptable carrier.

In one embodiment the present invention provides a composition comprising a first antibody or an antigen-binding fragment thereof that binds to a first epitope cluster of the SARS-CoV-2 spike RBD and a second antibody or an antigen-binding fragment thereof that binds to a second epitope cluster of the SARS-CoV-2 spike RBD, and a pharmaceutically acceptable carrier.

In one embodiment the present invention provides a composition as described herein wherein the first epitope cluster of the SARS-CoV-2 spike RBD and the second epitope cluster of the SARS-CoV-2 spike RBD do not overlap.

In one embodiment the present invention provides a composition as described herein, wherein the first antibody or an antigen binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133), and the second antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134).

In one embodiment the present invention provides a composition as described herein, wherein the first antibody or antigen binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133), and the second antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134), and further comprising a third antibody or an antigen binding fragment thereof comprising a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154).

In one embodiment the present invention provides a composition comprising a first antibody or an antigen-binding fragment thereof that binds to an epitope cluster 4 of the SARS-CoV-2 spike RBD and a second antibody or an antigen-binding fragment thereof that binds to an epitope cluster of the SARS-CoV-2 spike RBD overlapping with the ACE2 binding site of the spike protein, and a pharmaceutically acceptable carrier.

In one embodiment the present invention provides a composition as described herein wherein the antibody or an antigen-binding fragment thereof that binds to an epitope cluster 4 of the SARS-CoV-2 spike RBD.

In one embodiment the present invention provides a composition as described herein wherein the antibody or an antigen-binding fragment thereof that binds to an epitope cluster 4 of the SARS-CoV-2 spike RBD comprises a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154)

In one embodiment the present invention provides a composition as described herein wherein the antibody or an antigen-binding fragment thereof that binds to an epitope cluster 4 of the SARS-CoV-2 spike RBD comprises a HCDR1 of GFTFSDYY (SEQ ID NO: 24), a HCDR2 of ISGGSSFT (SEQ ID NO: 50), and a HCDR3 of ARSPPTGDSSDWYDSPAYNNYYMDV (SEQ ID NO: 76)

In one embodiment the present invention provides an isolated polynucleotide comprising a nucleic acid which encodes an antibody or an antigen-binding fragment as described herein.

In one embodiment the present invention provides a vector comprising a polynucleotide as described herein.

In one embodiment the present invention provides a host cell comprising a polynucleotide as described herein.

In one embodiment the present invention provides a method for prophylaxis or treatment of SARS-CoV-2 infection in a subject comprising administering an effective amount of an antibody or an antigen-binding fragment thereof as described herein.

In one embodiment the present invention provides a method for prophylaxis or treatment of SARS-CoV-2 infection in a subject comprising administering an effective amount of a composition as described herein.

A use of an antibody or an antigen-binding fragment thereof as described herein in the manufacture of a medicament for prophylaxis or treatment of SARS-CoV-2 infection in a subject.

In one embodiment the present invention provides a use of a composition as described herein in the manufacture of a medicament for prophylaxis or treatment of SARS-CoV-2 infection in a subject.

### BRIEF DESCRIPTION OF DRAWINGS

FIG. 1: Neutralisation and specificity of SARS-CoV-2 spike-specific mAbs. (A) Neutralisation of live SARS-CoV-2 virus (hCoV-19/Australia/VIC01/2020) was assessed for human mAbs recovered from convalescent COVID-19 individuals (n=69) or REGN10933 and REGN10987. Dashed line indicates 2ug/ml threshold used to define high potency. (B) Binding to recombinant SARS-CoV-2 trimeric spike, monomeric RBD, monomeric NTD or SARS-CoV trimeric spike was assessed by ELISA.

FIG. 2: Neutralisation and specificity of SARS-CoV-2 RBD and spike-specific WCSL mAbs from phage display. (A) Neutralisation of live SARS-CoV-2 virus (hCoV-19/Australia/VIC01/2020) was assessed for synthetic mAbs recovered from phage display (n=120). Dashed line indicates 2 ug/ml threshold used to define high potency. Binding to (B) recombinant SARS-CoV-2 trimeric spike, (C) monomeric SARS-CoV-2 RBD or (D) monomeric SARS-CoV RBD.

FIG. 3: Defining RBD binding epitopes for lead candidate antibodies. (A) Epitope binning competition BLI. Antibodies competing for RBD binding are shown in grey, non-competitive antibody pairs are shown in white. (B) crystal structures of lead candidate antibody fAb fragments in complex with the SARS-CoV-2 RBD (grey). The RBD is shown in surface representation and the footprint of the ACE2 binding site is highlighted in grey. Antibody variable domains are shown in cartoon representation and are coloured by cluster. (C) Surface representation of the SARS-CoV-2 RBD (grey) back face, top and front face (left to right) showing the binding footprints for each of the six identified epitope clusters. Footprints for the ACE2 and antibody binding sites are defined by residue contacts within 4 Å of the SARS-CoV-2 RBD.

FIG. 4: Structures of lead candidate fAbs with SARS-CoV-2 spike. Cryo-EM maps of fAb-spike complexes were lowpassed to 6 Å resolution with spike (light grey), RBDs (darker greys) and fAb shown in darker greys (A) WCSL129 (B) PDI 222 (C) PDI 210 (D) WCSL 119 (E) PDI 215 (F) PDI 93 (G) PDI 96.

FIG. 5: Protective efficacy of antibodies for prophylaxis in mice and hamsters. (A) Potently neutralising mAbs were administered to C57BL/6 mice (N=5 per group) via intraperitoneal injection at high (5 mg/kg), mid (1 mg/kg) and low (0.2 mg/kg) doses one day prior to aerosolised respiratory challenge with SARS-CoV-2. Virus within lung homogenates was quantified by limiting dilution (TCID50) at day 3 post-infection. (B) Spearman correlation between median viral load in the lung at 0.2 mg/kg treatment dose and in vitro microneutralisation activity (ng/ml 50% inhibitory concentration). (C) Two antibody cocktails were administered via intraperitoneal injection at 1 mg/kg, 0.2 mg/kg and 0.05 mg/kg doses one day prior to respiratory challenge, with Virus within lung homogenates assessed at day 3 post-infection. PDI 96 and PDI 222 were administered as single agents or as a cocktail via intraperitoneal injection to Syrian Golden hamsters (N=8 per group) at 5 mg/kg or 0.25 mg/kg one day prior to intranasal challenge with SARS-CoV-2. Four animals were sacrificed at day 3 post-infection for determination of viral loads. Remaining animals were sacrificed at day 7 post-infection for histopathology. (D) Weight loss in challenged animals over time. (E) Virus recoverable in cranial lung homogenates (PFU per 100 mg tissue). (F) Histopathological scoring of the lungs sections of challenged animals. Each animal trial represents a single experiment.

FIG. 6: Epitope resilience of mAbs in the context of SARS-CoV-2 variants. (A) Relative ELISA binding activity was determined for a panel of human mAbs (n=40) using recombinant trimeric spike proteins from wild-type and B.1.1.7, B.1.351 and P.1 variants of concern. (B) Comparison of neutralisation potencies (IC₅₀; ng/ml) for selected mAbs against wild-type and B.1.351 virus isolates. Bars represent geometric mean +/−SD titres of two assays. (C) Human antibody footprints for lead candidates showing heavy and light chain contacts on the RBD surface (light chain in lighter colour for each antibody). Contacts are shown for atoms within 4 Å of the RBD surface. Residues mutated in the variants of concern (VOC) are labelled and coloured in Blue-black, where the antibody footprint overlaps a VOC the contacted atoms are shown in violet.

FIG. 7 COVID-19 convalescent individuals subject to recovery and sequencing of spike-specific memory B cells.

FIG. 8: Gating scheme for the recovery of single spike-specific memory B cells from COVID-19 convalescent individuals.

FIG. 9: Heavy chain V-gene distribution of RBD—(n=97) or S-specific (non-RBD) mAbs (n=64) recovered from convalescent COVID-19 subjects.

FIG. 10: Table of binding and neutralisation characteristics for a subset of anti-RBD mAbs (n=12) with potent neutralising activity.

FIG. 11: Antibody ACE2 footprint overlaps, Antibody Centre of Mass and Antibody contracting CDR loops: (A) ACE2 footprint overlap (B) Axes showing antibody centre of mass and binding angle (C) Placements of antibody CDR loops contacting the SARS-CoV-2 RBD surface.

FIG. 12: Cryo-electron microscopy unsharpened maps of lead candidate antibodies. PDI 93, PDI 96, PDI 210, PDI 215, PDI 222, WCSL 119 and WCSL 129 coloured by local resolution. Highest resolution areas are coloured blue and lowest resolution areas are coloured red. Scales and FSC curves are shown below each map.

FIG. 13: Cryo-EM data processing workflow for SARS-CoV-2 spike-fAb complexes. Stages of data processing shown for (A) Spike+ POI 93. (B) Spike+ POI 96 (C) Spike+ PDI 210 (D) Spike+ POI 215 (E) Spike+ POI 222 (F) RBD+ POI 222 (G) Spike+ WCSL 119 and (H) Spike+ WCSL 129. Details of workflows are fully described in materials and methods.

FIG. 14: Epitope localisation and sequence alignments of VH1-58 stereotypic class of mAbs. Models depicting POI 222 or previously described VH-1-58 class mAbs COVOX253 (PDB: 7BEN) or S2E12 (PDB: 7K45) docked into the cryo-EM map of POI 222 RBD, revealing highly conserved binding epitopes on the RBD. Sequence alignment of selected VH1-58 and VK3-20 immunoglobulins indicating strong conservation of CDR-H3 length and dicysteine motifs, and near identical selection of VK3-20 rearrangements, define this stereotypic class.

FIG. 15: Viral titres in nasal swabs, nasal turbinates and caudal lung samples from hamsters treated prophylactically with human mAbs.

FIG. 16: Impact of LALA Fc mutations on in vivo protection in hamsters prophylactically administered high (5 mg/kg) and low (0.25 mg/kg) doses of PDI 96, PDI 222 or a two antibody cocktail prior to challenge with SARS-CoV-2.

FIG. 17: Comparison of the protective efficacy of a two (PDI 96/PDI 222) versus three antibody cocktail (PDI 96/PDI 222/PDI 215) in hamsters prophylactically administered high (5 mg/kg) and low (0.25 mg/kg) doses prior to challenge with SARS-CoV-2.

FIG. 18: Interactions of structurally characterised lead candidates with RBD residues mutated in variants B.1.1.7, B.1.351 and P1. (A) PDI 37 hydrogen bonds with RBD residue K417. (B) PDI 37 hydrogen bonds with residue N501. (C) PDI 42 hydrogen bond with RBD residue E484. (D) PDI 42 hydrogen bonds with RBD residue N501. (E) WCSL 119 forms two hydrogen bonds with the backbone of RBD residue E484. (F) WCSL 129 hydrogen bonds with RBD residue K417. (G) PDI 93 hydrogen bond with RBD residue E484. (H) PDI 210 VDW interaction with RBD residue E484. (I) PDI 210 VDW interaction with RBD residue N501. (J) PDI 215 salt bridge with RBD residue R82. (K) PDI 231 hydrogen bonds with RBD residue K417. (L) PDI 231 hydrogen bonds with RBD residue N501 and (M) PDI 222 forms a hydrogen bond with the backbone of RBD residue E484.

FIG. 19: (A) Relative RBD binding and (B) ACE-2-RBD inhibitory activity was assessed human mAbs in a high-throughput multiplex assay using a panel of single amino-acid variant RBD proteins. Activity was normalised to wild-type (Wuhan1) RBD. “>100”=low, shaded=intermediate, boxed=high binding or inhibitory activity.

FIG. 20: Characterisation of human mAbs that neutralise SARS-CoV-2.

FIG. 21: Diagrammatic representation of mutations in spike that define B.1.1.7, B1.351, B.617.2 and P1.

FIG. 22: Characterisation of human mAbs that neutralise SARS-CoV-2 variants Beta, Delta and Omicron (BA.1). Relative neutralisation is shown with No neutralisation” indicating no neutralisation, “intermediate” indicating intermediate neutralisation,” strong neutralisation: indicating strong neutralisation, and white indicating not done. Computationally predicted structural features in the HCDR3 are noted; C—C, intra HCDR3 disulphide bond; glycan, N-glycosylation site.

FIG. 23: Characterisation of human mAbs that neutralise SARS-CoV-2 Omicron variants BA.1, BA.2 and BA.4. Relative neutralisation is shown with “10000” indicating no neutralisation, shading indicating >3-fold loss of activity versus neutralisation of ancestral strain, boxes indicating strong neutralisation, n.d. indicating not done.

### DETAILED DESCRIPTION

The present invention is based in part on the characterisation of human monoclonal antibodies that neutralise SARS-CoV-2, and the characterisation of antibody epitopes across the viral spike protein which consistently yield robust in vitro and in vivo neutralisation outcomes.

Accordingly, in one embodiment, the present invention provides an antibody or an antigen-binding fragment thereof that neutralises SARS-CoV-2.

As used herein the term “antibody” includes a polypeptide comprising an immunoglobulin variable region which specifically recognizes an epitope on an antigen, for example, SARS-CoV-2 spike RBD or NTD. The term “antibody” also includes an antibody fragment that maintains the ability to bind to an epitope on an antigen. Preferred “antigen binding fragments” are an Fab fragment, an Fab′ fragment, an F(ab′)2 fragment, an Fv fragment, a single chain antibody, a single chain Fv fragment, a disulfide stabilized Fv protein, or a dimer of a single chain Fv fragment. Methods for producing these fragments and antibodies are well known in the art (see for example, Harlow & Lane: Antibodies, A Laboratory Manual, Cold Spring Harbor Laboratory, 1988).

Antibodies are usually comprised of two identical heavy chains and two identical light chains, each of which has a variable region at its N-terminus (V_{H} and V_{L} region). Usually, a VH and a VL region will combine to form the antigen binding site. However, single domain antibodies, where only one variable region is present and binds to the antigen, have also been described.

Typically, an antibody contains two heavy and two light chains, connected by disulfide bonds. There are 5 major isotypes of antibodies (IgG, IgM, IgE, IgA, IgD), some of which occur as multimers of the basic antibody structure. The isotype is determined by the constant region of the heavy chains. There are two types of light chains, lambda and kappa.

The term “antibody” as used herein includes intact antibodies (also known as full length antibodies, or antibodies which comprise both heavy and light chain variable and constant domains), as well as variants and portions thereof that retain antigen binding. This includes fragments of antibodies such as Fab fragments, F(ab′)2 fragments, Fab′ fragments, single chain Fv fragments, or disulfide-stabilized Fv fragments. Thus, the term “antibody or an antigen-binding fragment thereof” and the term “antibody” alone are used herein interchangeably to refer to an antibody and antigen-binding fragments thereof.

Each heavy and light chain consists of a variable region and a constant region. The variable regions contain framework residues and hypervariable regions, which are also called complementarity determining regions or CDRs.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs.

As used herein, the term “complementarity determining regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. The extent of the framework residues and CDRs can be determined according to a number of well-known numbering schemes including Kabat, Chothia, IMGT etc. The CDR regions are important in binding to the epitope and therefore determine the specificity of the antibody.

As used herein, “framework regions” (FRs) are those variable domain residues other than the CDR residues.

As used herein, a “monoclonal antibody” is an antibody produced by a single clone of B lymphocytes, or by a cell line engineered to express a single antibody.

As used herein, a “chimeric antibody” is an antibody with the variable regions from one species grafted onto the constant regions from a different species. A “humanized” antibody is an antibody where CDR regions from a different species, e.g. a mouse monoclonal antibody, are grafted into the framework of a human antibody. Analogously, a “murinized” antibody is an antibody where the CDR regions from a different species, e.g. a human monoclonal antibody, are grafted into the framework of a mouse antibody. A human antibody is an antibody that is wholly derived from human, i.e. human CDRs in a human framework and any constant region suitable for administration to a human.

As used herein, “antigen binding fragment” refers to any fragment of an antibody that retains the ability to specifically bind the epitope of the antigen that the antibody binds to. These include but are not limited to Fab, F(ab′)2, or single chain Fv fragments.

As used herein, “binding affinity” refers to the affinity of the antibody to its antigen. It can be measured by a variety of techniques, e.g. surface plasmon resonance based technology (BiaCore®), or other methods, including bio-layer interferometry as described herein.

As used herein, an “epitope” is an antigenic determinant, it is defined by the residues or particular chemical structures that the antibody makes contact with on the antigen.

As used herein, “sequence identity” relates to the similarity of amino acid sequences. The best possible alignment of two sequences is prepared, and the sequence identity is determined by the percentage of identical residues. Standard methods are available for the alignment of sequences, e.g. algorithms of Needleman and Wunsch (J Mol Biol (1970) 48, 443), Smith and Waterman (Adv Appl Math (1981) 2, 482), Pearson and Lipman (Proc Natl Acad Sci USA (1988) 85, 2444), and others. Suitable software is commercially available, e.g. the GCG suite of software (Devereux et al (1984), Nucl Acids Res 12, 387), where alignments can be produced using, for example, GAP or BESTFIT with default parameters, or successors thereof. The Blast algorithm, originally described by Altschul et al (J. Mol. Biol. (1990) 215, 403), but further refined to include gapped alignments (Blast 2), available from various sources such as the EBI, NCBI, will also produce alignments and calculate the % identity between two sequences.

As used herein the term “neutralises SARS-CoV-2” refers to reducing the infectivity of SARS-CoV-2, for example, by inhibiting the attachment of SARS-Co-2 to receptors on host cells. In a specific embodiment, the binding molecules of the invention prevent SARS-Co-2 from infecting host cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to infection of host cells by SARS-CoV in the absence of said binding molecules. Neutralisation can for instance be measured as described herein.

In one embodiment, neutralisation is determined by a method as described herein, such as neutralisation assays with suitable cells, such as Vero cells. Example 1 sets out a microneutralisation assay with ELISA-based read out.

Importantly, the present inventors have demonstrated herein that the extent of viral suppression in vivo correlated with in vitro measurements of neutralising activity but not binding affinity, suggesting functional potency is a key defining metric for protective efficacy.

In another embodiment, assessment of mAb binding specificity and can be determined by any suitable means, for example using ELISA or a microneutralisation assay with ELISA-based read out.

In one embodiment, assessment of mAb binding affinity can be determined by any suitable means, for example using bio-layer interferometry, as described herein.

As used herein, “specific binding” refers to the binding to substantially only a single antigen, e.g. the spike RBD.

The SARS-CoV-2 spike protein mediates attachment of the virus to host cell-surface receptors and fusion between virus and cell membranes. Consequently, mutations that affect the antigenicity of the spike protein are of particular importance.

The spike protein is a transmembrane glycoprotein, which forms homotrimers on the surface of the virion. The SARS-CoV-2 spike protein is highly glycosylated, with 66 potential N-glycosylation sites per trimer. The SARS-CoV-2 spike protein is post-translationally cleaved by mammalian furin into two subunits: S1 and S2. The S1 subunit largely consists of the amino-terminal domain and the receptor-binding domain (RBD), and is responsible for binding to the host cell-surface receptor, ACE2, whereas the S2 subunit includes the trimeric core of the protein and is responsible for membrane fusion.

The present inventors have identified herein antibodies which bind six epitope clusters of the spoke RBD, which all display partial overlap of binding sites with that of ACE2.

Preferably, the antibody used in the context of the invention binds human SARS-CoV-2 RBD with a KD of less than 20 nM, more preferably less than 10 nM, more preferably less than 5 nM, even more preferably less than 1 nM using bio-layer interferometry

Preferably, the antibody used in the context of the invention binds human SARS-CoV-2 spike with a KD of less than 1 nM, more preferably less than 0.5 nM, more preferably less than 0.1 nM, even more preferably less than 0.01 nM using bio-layer interferometry.

As used herein, “SARS-CoV-2” refers to coronaviruses related to the Severe Acute Respiratory Syndrome (SARS) virus, and named by International Committee on Taxonomy of Viruses (ICTV) as severe acute respiratory syndrome coronavirus 2 (SARS-CoV-2). As used herein “COVID-19” refer to the disease caused by SARS-CoV-2.

SARS-CoV-2 isolate sequences are available via the Global Initiative on Sharing All Influenza Data (GISAID) database.

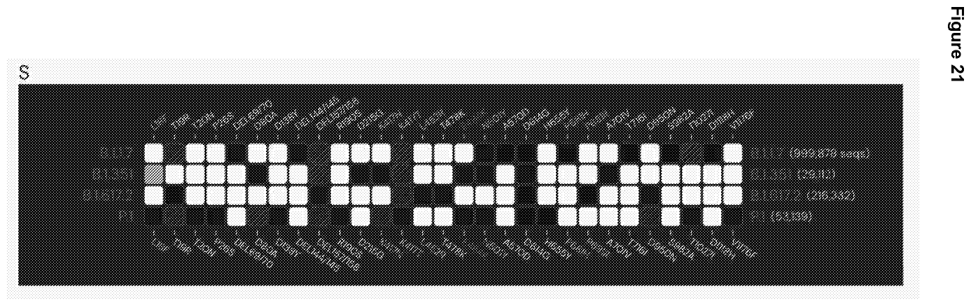

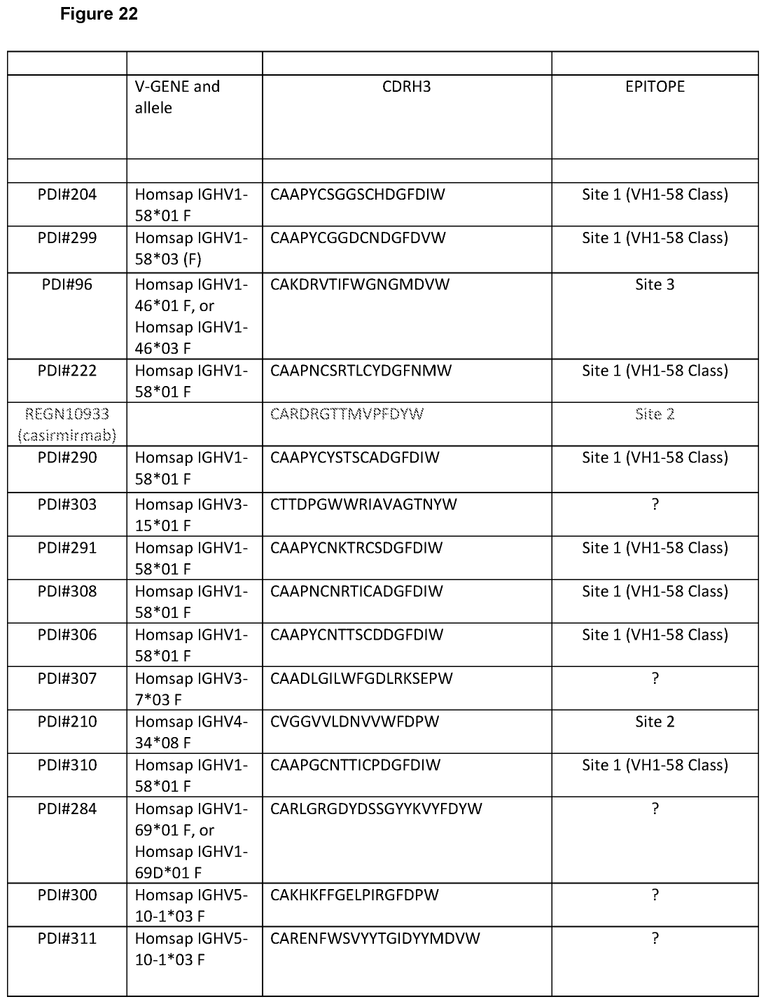

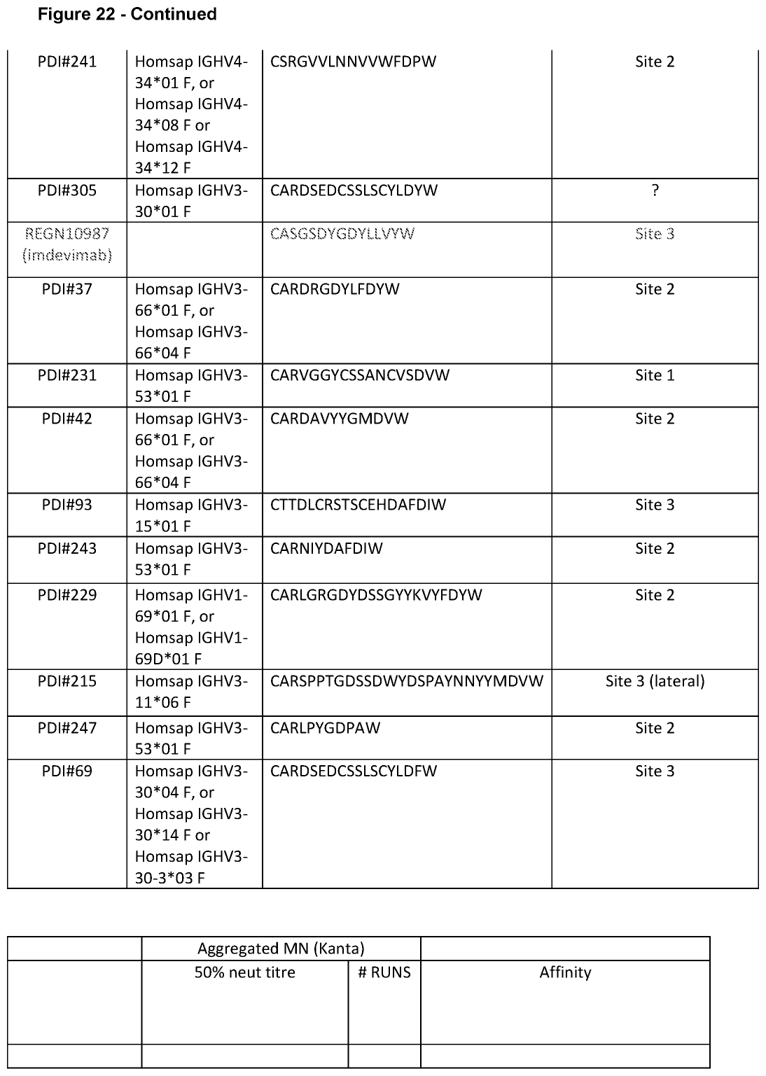

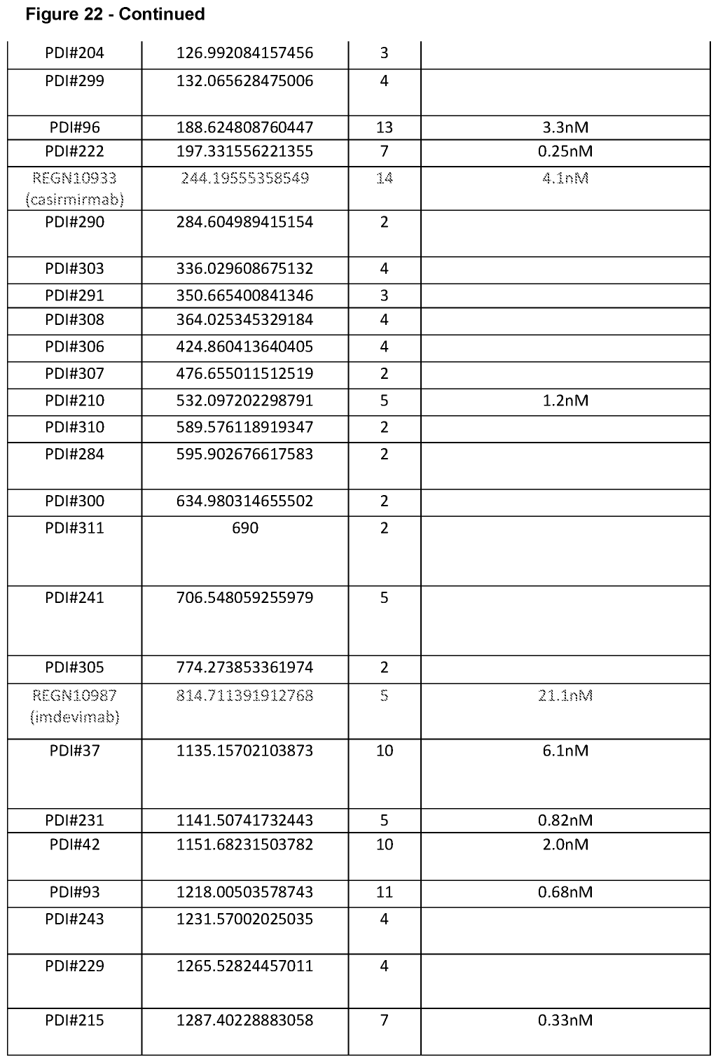

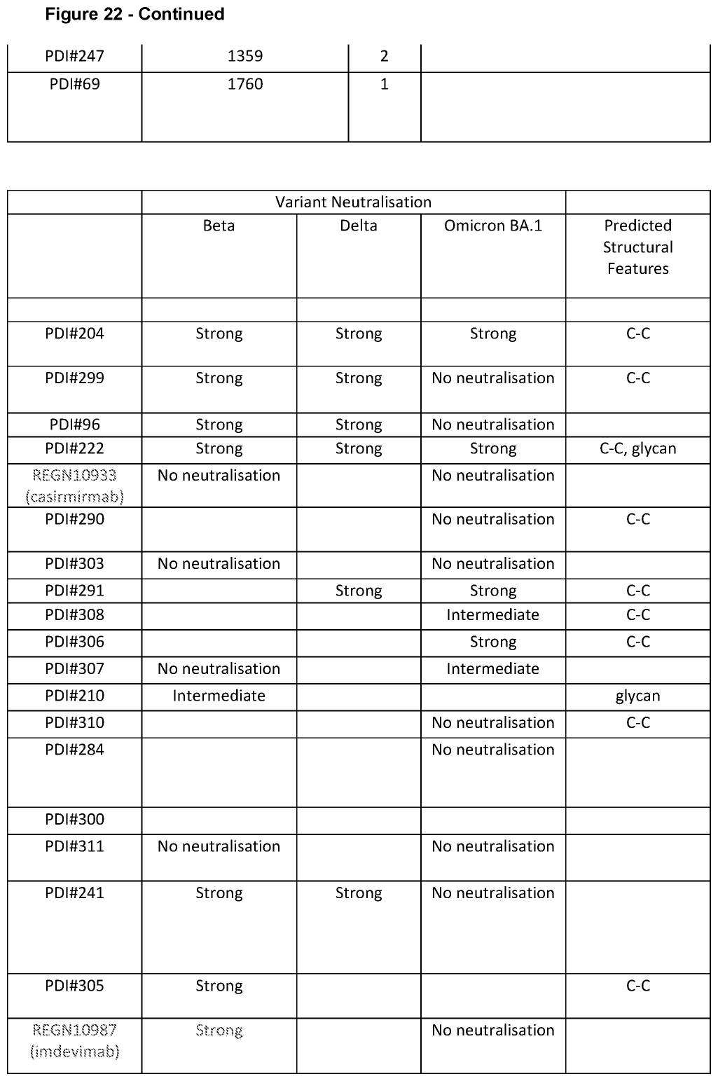

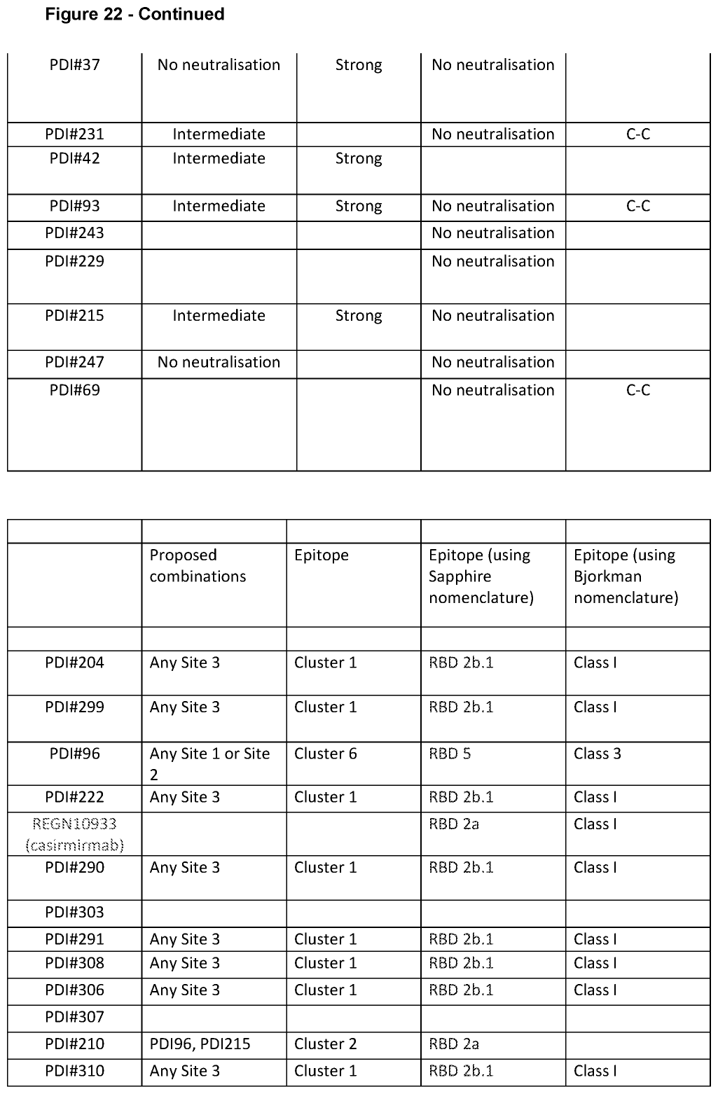

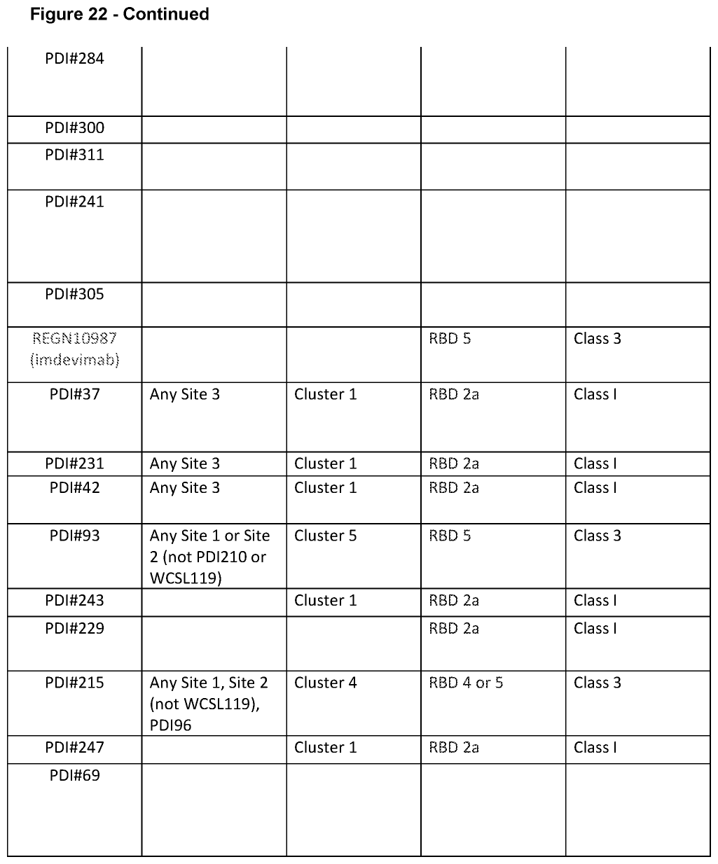

Previous studies have characterised escape mutations—mutations that emerge in virus populations exposed to either mAbs or convalescent plasma containing polyclonal antibodies—have characterised that substitution or deletion of residue 140, or substitutions of residues 148, 140, 150, 151, 345, 346, 352, 378, 406, 408, 417, 439, 441, 443, 444, 445, 446, 447, 448, 449, 450, 452, 453, 455, 456, 458, 472, 473, 474, 476, 477, 478, 479, 483, 484, 485, 486, 487, 489, 490, 493, 494, 496, 499, 501, 503, 504 and 519 of the spike RBD has an effect on mAb binding and/or polyclonal sera binding, and/or emergence of escape mutants to mAbs and/or polyclonal sera binding. FIG. 22 shows the mutations in spike that define B.1.1.7, B1.351, B.617.2 and P1.

The present inventors have demonstrated in the Examples that the antibodies described herein are able to neutralise SARS-CoV-2 isolates of different variants in plaque reduction neutralisation, and in vivo in infected mice and hamsters.

In one embodiment, the present invention provides an antibody or an antigen-binding fragment thereof that neutralises a SARS-CoV-2 isolate selected from the group consisting of hCoV-19/Australia/VIC01/2020, CoV/Australia/QLD/1520/2020, hCoV-19/Australia/VIC2089/2020 and SARS-CoV-2/human/USA/WA-CDC-WA1/2020.

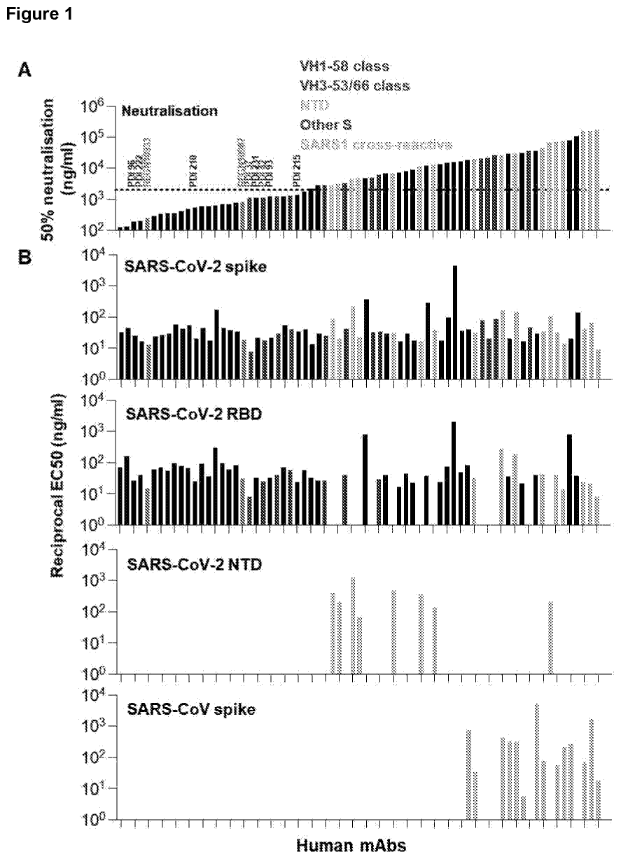

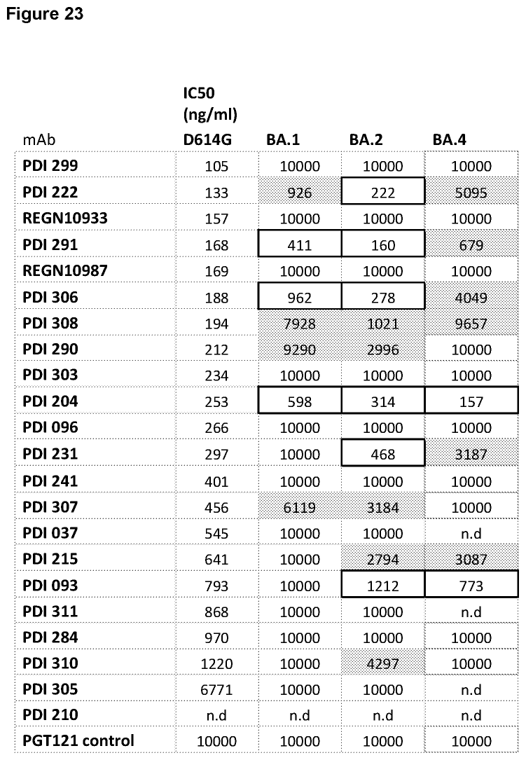

Importantly, the present inventors have demonstrated a number of antibodies surprisingly showed an IC50 and IC100 in microneutralisation assays less than therapeutic monoclonal antibodies that have been approved by the United States Food and Drug Administration. For example, FIG. 1 and FIG. 6 demonstrate PD196, PD1222, and PD1210 all had a lower IC50 in microneutralisation assay than the IC50 of REGN10987, and PD196 and PD1222 have a lower IC50 in microneutralisation assay than the IC50 of REGN10933. PD196, PD1222, and PD1210 all had a lower IC100 in microneutralisation assay than the IC100 of REGN10987, and PD196 and PD1222 a lower IC100 in microneutralisation assay than the IC100 of REGN10933 (data not shown). FIG. 23 demonstrates numerous antibodies with a lower IC50 in microneutralisation assay than the IC50 of REGN10987 or REGN10933.

In one embodiment, the present invention provides an antibody or an antigen-binding fragment thereof that neutralises a SARS-CoV-2 variant selected from the group consisting of B.1.1.7 (Alpha), B.1.351 (Beta), B.1.617.2 (Delta), P.1 (Gamma), B.1.427 (Epsilon), B.1.429 (Epsilon), B.1.525 (Eta), B.1.526 (Iota) B.1.617.1 (Kappa), B.1.617.3 and B.1.1.529 (Omicron).

SARS-CoV-2 variants include:
a. B.1.1.7 (Alpha), which has the following Spike Protein Substitutions: 69del, 70del, 144del, (E484K*), (S494P*), N501Y, A570D, D614G, P681H, T7161, S982A, D1118H (K1191 N*).
b. B.1.351 (Beta), which has the following Spike Protein Substitutions: D80A, D215G, 241del, 242del, 243del, K417N, E484K, N501Y, D614G, A701V.
c. B.1.617.2 (Delta), which has the following Spike Protein Substitutions: T19R, (V70F*), T951, G142D, E156−, F157−, R158G, (A222V*), (W258L*), (K417N*), L452R, T478K, D614G, P681R, D950N.
d. P.1 (Gamma), which has the following Spike Protein Substitutions: L18F, T20N, P26S, D138Y, R190S, K417T, E484K, N501Y, D614G, H655Y, T10271.
e. B.1.427 (Epsilon), which has the following Spike Protein Substitutions: L452R, D614G
f. B.1.429 (Epsilon), which has the following Spike Protein Substitutions: S131, W152C, L452R, D614G.
g. B.1.525 (Eta), which has the following Spike Protein Substitutions: A67V, 69del, 70del, 144del, E484K, D614G, Q677H, F888L.
h. B.1.526 (Iota) which has the following Spike Protein Substitutions: L5F, (D80G*), T951, (Y144-*), (F157S*), D253G, (L452R*), (S477N*), E484K, D614G, A701V, (T859N*), (D950H*), (Q957R*).
i. B.1.617.1 (Kappa), which has the following Spike Protein Substitutions: (T951), G142D, E154K, L452R, E484Q, D614G, P681R, Q1071H.
j. B.1.617.3, which has the following Spike Protein Substitutions: T19R, G142D, L452R, E484Q, D614G, P681R, D950N.
k. B.1.1.529 (Omicron BA.1), which has the following Spike Protein Substitutions: G339D, S371L, S373P, S375F, K417N, N440K, G446S, S477N, T478K, E484A, Q493R, G496S, Q498R, N501Y, Y505H.
l. B.1.1.529 (Omicron BA.2), which has the following Spike Protein Substitutions: G339D, S371 F, S373P, S375F, T376A, D405N, R408S, K417N, N440K, S477N, T478K, E484A, Q493R, Q498R, N501Y, Y505H.
m. B.1.1.529 (Omicron BA.3), which has the following Spike Protein Substitutions: G339D, S371F, S373P, S375F, D405N, K417N, N440K, G446S, S477N, T478K, E484A, Q493R, Q498R, N501Y, Y505H.
n. B.1.1.529 (Omicron BA.4), which has the following Spike Protein Substitutions: G339D, S371 F, S373P, S375F, T376A, D405N, R408S, K417N, N440K, L452R, S477N, T478K, E484A, F486V; R493Q, Q498R, N501Y, Y505H.
o. B.1.1.529 (Omicron BA.5), which has the following Spike Protein Substitutions: G339D, S371 F, S373P, S375F, T376A, D405N, R408S, K417N, N440K, L452R, S477N, T478K, E484A, F486V; R493Q, Q498R, N501Y, Y505H.
p. B.1.1.529 (Omicron BA.2.75), which has the following Spike Protein Substitutions: G339H, S371 F, S373P, S375F, T376A, D405N, R408S, K417N, N440K, G446S, N460K, S477N, T478K, E484A, Q493, Q498R, N501Y, Y505H.

Polyclonal sera from patients infected by wild-type SARS-CoV-2 or recipients of current mRNA vaccines have been demonstrated to show a substantial loss in neutralizing activity against both Omicron BA.1 with a R346K mutation (BA.1.1) and Omicron BA.2, with drops comparable to that reported for BA.1.

On 25 Jan. 2022, the US FDA revised its emergency use authorization (EUA) for two monoclonal antibody combinations to treat mild-to-moderate COVID-19 because they are not effective against the Omicron variant. The drugs for which the EUA was revised were 1) LY-CoVO16 (marketed as etesevimab) combined with LY-CoV555 (marketed as bamlanivimab), from Eli Lilly, which was authorized for use in February 2021, and 2) casirivimab (REGN10933) combined with imdevimab (REGN10987), from Regeneron, which received an EUA in November 2020.

REGN10987 has been shown to lose neutralizing activity against omicron/BA.1 (NC928) and omicron/BA.1.1 (NC929). BA.1.1, a subvariant of BA.1, has the R346K mutation in the S protein. Furthermore, a live-virus focus reduction neutralization test (FRNT) showed that both LY-CoV016 (marketed as etesevimab) and LY-CoV555 (marketed as bamlanivimab), individually and in combination, lost neutralizing activity against omicron/BA.2.

The combination of REGN10987 and REGN10933 (marketed as casirivimab) also inhibited omicron/BA.2 but did not inhibit omicron/BA.1 or omicron/BA.1.1. However, the FRNT50 (the titer of monoclonal antibodies required for a 50% reduction in the number of infectious foci) value of this combination therapy was higher by a factor of 43.0 to 143.6 for omicron/BA.2 than for an ancestral strain—SARS-CoV-2/UT-NC002-1T/Human/2020/Tokyo (NC002)—and other variants of concern (i.e., the alpha [B.1.1.7], beta [B.1.351], gamma [P.1], and delta [B.1.617.2] variants).

Other work has demonstrated that REGN10933, REGN10987, REGN10933+ REGN10987, LY-CoV555, LY-CoVO16, LY-CoV555+LY-CoVO16, AZD8895, AZD1061 lacked detectable neutralizing titer against BA.1, that the AZD8895+AZD1061 mixture and the Vir-7831 monoclonal antibody neutralizing titers were decreased by 359-fold and 11-fold compared to the parental D614G, and that REGN10933, REGN10987, REGN10933+ REGN10987, LY-CoV555, LY-CoV016, and LY-CoV555+LY-CoV016 lacked detectable neutralizing titer against BA.2 while Vir-7831 was active against BA.2, albeit with decreased titer. Celltrion (CT-P59) completely lost neutralizing activity against B.1.1.529 virus in both Vero-TMPRSS2 and Vero-hACE2-TMPRSS2 cells, whereas others were reduced (AstraZeneca's combination COV2-2196 and COV2-2130—the parent mAbs of AZD8895 and AZD1061, which shows a ˜12-fold decrease). The neutralization potency of AZD1061, AZD8895 and BRII-196 has also been demonstrated to be greatly undermined by Omicron.

The present inventors have demonstrated in FIG. 19 that the G446S Spike Protein Substitution of Omicron BA.1 is critical to REGN10987 binding, and that PDI 96, PDI 222, and PDI 210 showed better binding to RBD G446S than REGN10987.

Importantly, FIG. 22 demonstrates a) many antibodies displayed a significant loss of neutralisation potency against Omicron BA.1, and b) many antibodies displayed no significant loss of neutralisation potency against Omicron BA.1. For example, REGN10933 and REGN10987 were demonstrated to not neutralise Omicron BA.1. In contrast, PDI 204, PDI 222, PDI 291 and PDI 306 strongly neutralised Omicron BA.1, and PDI 308 and PDI 307 demonstrated intermediate neutralisation of Omicron BA.1.

FIG. 23 demonstrates a) many antibodies displayed a significant loss of neutralisation potency against Omicron BA.1, BA.2 and/or BA.4, and b) many antibodies displayed no significant loss of neutralisation potency against Omicron BA.1, BA.2 and/or BA.4. For example, REGN10933 and REGN10987 were demonstrated to not neutralise Omicron BA.1, BA.2 or BA.4. In contrast, PDI 222, PDI 291, PDI 306 and PDI 204, PDI 231, PDI 093, neutralised at least one of Omicron BA.1, BA.2 and/or BA.4.

Accordingly, in one embodiment, the present invention provides an antibody or an antigen-binding fragment thereof that neutralises more than one SARS-CoV-2 variant selected from the group consisting of B.1.1.7 (Alpha), B.1.351 (Beta), B.1.617.2 (Delta), P.1 (Gamma), B.1.427 (Epsilon), B.1.429 (Epsilon), B.1.525 (Eta), B.1.526 (Iota), B.1.617.1 (Kappa), B.1.617.3 and B.1.1.529 (Omicron).

In a preferred embodiment, the present invention provides an antibody or an antigen-binding fragment thereof as described herein, wherein the antibody or an antigen-binding fragment thereof neutralises more than one B.1.1.529 (Omicron) variant. For example, Omicron BA.1, Omicron BA.2 and/or Omicron BA.4.

In one embodiment the present invention provides an antibody or an antigen-binding fragment thereof that neutralises SARS-CoV-2 isolate hCoV-19/Australia/VIC01/2020.

FIG. 22 demonstrates a number of antibodies strongly neutralised the Delta, Beta and Omicron variants.

In one embodiment the present invention provides an antibody or an antigen-binding fragment thereof that neutralises SARS-CoV-2 variant Delta.

In one embodiment the present invention provides an antibody or an antigen-binding fragment thereof that neutralises SARS-CoV-2 variant Beta.

In one embodiment the present invention provides an antibody or an antigen-binding fragment thereof that neutralises SARS-CoV-2 variant Omicron.

As used herein, “treating” or “treatment” includes the reduction of any symptoms associated with COVID-19. The term includes therapeutic treatment as well as prophylactic or preventative measures to cure or halt or at least retard disease progress. Those in need of treatment include those already inflicted with a condition resulting from infection with SARS-CoV-2 as well as those in which infection with SARS-CoV-2 is to be prevented. Subjects partially or totally recovered form infection with SARS-CoV-2 might also be in need of treatment. Prevention encompasses inhibiting or reducing the spread of SARS-CoV-2 or inhibiting or reducing the onset, development or progression of one or more of the symptoms associated with infection with SARS-CoV-2.

“Preventing” or “prevention” or “prophylaxis” includes the prevention of any symptoms associated with COVID-19 including the deterioration of the disease.

As explained in more detail in the attached examples, the present inventors have characterised a diverse panel of 92 SARS-CoV-2 neutralising human mAbs including comprehensive structural analysis of a subset.

In one embodiment, the present invention provides an antibody or an antigen-binding fragment thereof as described herein, wherein the antibody or an antigen-binding fragment thereof that neutralises SARS-CoV-2 with an IC50 of less than 200ug/ml.

In one embodiment, the present invention provides an antibody or an antigen-binding fragment thereof as described herein, wherein the antibody or an antigen-binding fragment thereof that neutralises SARS-CoV-2 with an IC50 of less than 100ug/ml.

In one embodiment, the present invention provides an antibody or an antigen-binding fragment thereof as described herein, wherein the antibody or an antigen-binding fragment thereof that neutralises SARS-CoV-2 with an IC50 of less than 50ug/ml.

In one embodiment, the present invention provides an antibody or an antigen-binding fragment thereof as described herein, wherein the antibody or an antigen-binding fragment thereof that neutralises SARS-CoV-2 with an IC50 of less than 10ug/ml.

In one embodiment, the present invention provides an antibody or an antigen-binding fragment thereof as described herein, wherein the antibody or an antigen-binding fragment thereof that neutralises SARS-CoV-2 with an IC50 of less than 2ug/ml.

In one embodiment the antibody or an antigen-binding fragment thereof that neutralises SARS-CoV-2 with an IC50 of less than 2ug/ml comprises a heavy chain variable region sequence of QVQLVQSGPEVKKPGTSVKVSCKASGFTFSNSAVQWVRQARGQRLEWIGWIVVGS GNTDYAQKFQERVTITRDMSTSTAYMELSSLRSEDTAVYFCAAPYCGGDCNDGFDV WGQGTMVTVSS (SEQ ID NO: 244) and comprises a light chain variable region sequence of

(SEQ ID NO: 270)
EIVLTQSPGSLSLSPGERATLSCRASQSVRSSYLAWYQQKLGQAPGLFI
YSTSRRATGIPDRFSGSGSGTDFTLTISRLEPEDFAVYYCQQYGSSPWT
FGQGTRVEIK.

In another embodiment, the antibody or antigen binding fragment thereof comprises a heavy chain variable domain having at least 90% identity to the amino acid sequence of SEQ ID NO: 244 and a light chain variable domain having at least 90% identity to the amino acid of SEQ ID NO: 270.

In one embodiment the antibody or an antigen-binding fragment thereof that neutralises SARS-CoV-2 with an IC50 of less than 2ug/ml comprises a heavy chain variable region sequence of LVQLVQSGAEVKKPGASVKISCKASGYTFTSYYMHWVRQAPGQGLEWMGIINPSG GGTSYAQKFQGRVTMTRDTSTSTVYMELTSLRSDDTAVYYCAKDRVTIFWGNGMD VWGQGTTVTVSS (SEQ ID NO: 245) and comprises a light chain variable region sequence of

(SEQ ID NO: 271)
DIVMTQSPVSLAVSLGERATIKCKSSQSVLYSSNNKNYLGWYQQKPGQP
LRLLIYWASTRESGVPDRFSGSGSGTDFTLTISSLQAEDVAVYYCHQYS
TTPLTFGGGTKVEIK.

In another embodiment, the antibody or antigen binding fragment thereof comprises a heavy chain variable domain having at least 90% identity to the amino acid sequence of SEQ ID NO: 245 and a light chain variable domain having at least 90% identity to the amino acid of SEQ ID NO: 271.

In one embodiment the antibody or an antigen-binding fragment thereof that neutralises SARS-CoV-2 with an IC50 of less than 2ug/ml comprises a heavy chain variable region sequence of QVQLVQSGPEVKKPGTSVKVSCKASGFTFSSSAVQWVRQARGQRLEWIGWIVVGS GNANYAPRFQERVTITRDMSTNTAYMELSSLRSEDTAVYYCAAPNCSRTLCYDGFN MWGQGTMVTVSS (SEQ ID NO: 246) and comprises a light chain variable region sequence of

(SEQ ID NO: 272)
EIVLTQSPGSLSLSPGERATLSCRASQSVRSSYLGWYQQKPGQAPRLLI
YGASSRATGIPDRFSGSGSETDFTLTISRLEPEDFAVYYCQQYDSSPWT
FGQGTKVEIK.

In another embodiment, the antibody or antigen binding fragment thereof comprises a heavy chain variable domain having at least 90% identity to the amino acid sequence of SEQ ID NO: 246 and a light chain variable domain having at least 90% identity to the amino acid of SEQ ID NO: 272.

In one embodiment the antibody or an antigen-binding fragment thereof that neutralises SARS-CoV-2 with an IC50 of less than 2ug/ml comprises a heavy chain variable region sequence of QVQLAQSGPEVKKPGTSVKVSCKASGFTFDLSAVQWVRQARGQRLEWIGWIAVGS GNTDYAQKFQERVTLTRDMSTSTAYMELSSLRSEDTAVYYCAAPYCYSTSCADGFD IWGQGTMVTVSS (SEQ ID NO: 247) and comprises a light chain variable region sequence of

(SEQ ID NO: 273)
EIVLTQFPFSLSLSPGERATLSCRASQSVRSGYLAWYQQKPGQAPRLLI
DGTSSRATGIPDRISGSGFGTHFTLTISRVEPEDFAVYYCQQYGSSPWT
FGQGTKVEIK.

In another embodiment, the antibody or antigen binding fragment thereof comprises a heavy chain variable domain having at least 90% identity to the amino acid sequence of SEQ ID NO: 247 and a light chain variable domain having at least 90% identity to the amino acid of SEQ ID NO: 273.

In one embodiment the antibody or an antigen-binding fragment thereof that neutralises SARS-CoV-2 with an IC50 of less than 2ug/ml comprises a heavy chain variable region sequence of EVQLVESGGGLVKPGGSLRLSCAASGFTFSNAWMSWVRQAPGKGLEWVGHIKSKT DGGTTDYGAPVKGRFTISRDDSKNTLFLQMNSLKTEDTAVYYCTTDPGWWRIAVAG TNYWGQGTLVTVSS (SEQ ID NO: 248) and comprises a light chain variable region sequence of

(SEQ ID NO: 274)
DIQMTQSPFSLSASVGDRVTITCRASQSISSYLNWFQQKPGKAPKLLIY
AASSLQSGVPSRFSGSGSGTDFTLTISSLQPEDFATYYCQQSDSSPPTF
GQGTKVEFK.

In another embodiment, the antibody or antigen binding fragment thereof comprises a heavy chain variable domain having at least 90% identity to the amino acid sequence of SEQ ID NO: 248 and a light chain variable domain having at least 90% identity to the amino acid of SEQ ID NO: 274.

In one embodiment the antibody or an antigen-binding fragment thereof that neutralises SARS-CoV-2 with an IC50 of less than 2ug/ml comprises a heavy chain variable region sequence of QVQLVQSGPEVKKPGTSVKVSCKASGFTFTLSAVQWVRLARGQRLEWIGWIVPGS GNVNYAQKFQERVTITRDMSTNTDYMELSSLRSEDTAVYYCAAPYCNKTRCSDGFD IWGQGTMVTVSS (SEQ ID NO: 249) and comprises a light chain variable region sequence of

(SEQ ID NO: 275)
EIVLTQSPGSLSLSPGERATLSCRASQSVSSSYLAWYQQKPGQAPRLLI
YGASSRASGIPDRFSGSGSGTDFTLTISRLEPEDFAVYYCQQYGSSLFT
FGPGTKVDIK.

In another embodiment, the antibody or antigen binding fragment there comprises a heavy chain variable domain having at least 90% identity to the amino acid sequence of SEQ ID NO: 249 and a light chain variable domain having at least 90% identity to the amino acid of SEQ ID NO: 275.

In one embodiment the antibody or an antigen-binding fragment thereof that neutralises SARS-CoV-2 with an IC50 of less than 2ug/ml comprises a heavy chain variable region sequence of QVQLVQSGPEVKKPGTSVKVSCKASGFTFTSSAVQWVRQARGQRLEWIGWIVVGS GNTNYAQKFQERVTITRDMSTTTAYMEVSSLRSEDTAVYYCAAPNCNRTICADGFDI WGQGTIVTVSS (SEQ ID NO: 250) and comprises a light chain variable region sequence of

(SEQ ID NO: 276)
EIGLTQSPCTVSLSPWERTTLSCRASQSVRSSYLAWYQQKPGQAPRLLI
CATSSRATGIPDRFSGSGSGTDFTLTISRLEPEDFAVYYCQQYGSSPWT
FGQGTKVEIK.

In another embodiment, the antibody or antigen binding fragment thereof comprises a heavy chain variable domain having at least 90% identity to the amino acid sequence of SEQ ID NO: 250 and a light chain variable domain having at least 90% identity to the amino acid of SEQ ID NO: 276.

In one embodiment the antibody or an antigen-binding fragment thereof that neutralises SARS-CoV-2 with an IC50 of less than 2ug/ml comprises a heavy chain variable region sequence of KIQLVQSGPEVKKPGTSVKVSCKASGFTFTTSAVQWVRQARGQRLEWIGWIAVGSG NTEYEQKFQERVTITRDMSTSTAYMELNSLKSEDTAVYYCAAPYCNTTSCDDGFDI WGQGTMVTVSS (SEQ ID NO: 251) and comprises a light chain variable region sequence of

(SEQ ID NO: 277)
EIVLTQSPGTLSLSPGERATLSCRASQSVRSNYLAWYQQKPGQAPRVLI
YGASSRATGIPDRFSGSGSGTDFTLTISRLEPEDFAVYHCQQYGSSPWT
FGQGTKVEIK.

In another embodiment, the antibody or antigen binding fragment thereof comprises a heavy chain variable domain having at least 90% identity to the amino acid sequence of SEQ ID NO: 251 and a light chain variable domain having at least 90% identity to the amino acid of SEQ ID NO: 277

In one embodiment the antibody or an antigen-binding fragment thereof that neutralises SARS-CoV-2 with an IC50 of less than 2ug/ml comprises a heavy chain variable region sequence of EVQLVESGGGLVQPGGSLRLSCAASGFIFNRYWMTWVRQAPGKGLEWVANIKQDG SEKYYVDSVKGRFTISRDNAKNSLYLQMNSLRAEDTAVYYCAADLGILWFGDLRKSE PWGQGTLVTVSS (SEQ ID NO: 252) and comprises a light chain variable region sequence of

(SEQ ID NO: 278)
DIQMTQSPFSLSASVGDRVSITCRASQGISNSLAWYQQKPGKAPKLLLY
AASTLESGVPSRFSGSGSGTDFTLTISSLQPEDFATYYCQEYYSLRTFG
QGTKVEIK.

In another embodiment, the antibody or antigen binding fragment thereof comprises a heavy chain variable domain having at least 90% identity to the amino acid sequence of SEQ ID NO: 252 and a light chain variable domain having at least 90% identity to the amino acid of SEQ ID NO: 278.

In one embodiment the antibody or an antigen-binding fragment thereof that neutralises SARS-CoV-2 with an IC50 of less than 2ug/ml comprises a heavy chain variable region sequence of QVQLQQWGAGLLKPSETLSLTCVVYGETLSGYYWTWIRQSPGKGLEWIGEISLRGT ANYNPSLKSRVTLSVEASKNQFSLKMTSVTAADTAVYYCVGGVVLDNVVWFDPWG QGIPVTVSL (SEQ ID NO: 253) and comprises a light chain variable region sequence of

(SEQ ID NO: 279)
ESVLTQSPVSLSLSPGERATLSCRASQSVGTYLAWYQHRPGQAPRLLIY
NASKRATGIPARFSGSGSGTDFTLTISSLEPEDLAVYFCQQRSNWLTFG
GGTKVEIK.

In another embodiment, the antibody or antigen binding fragment thereof comprises a heavy chain variable domain having at least 90% identity to the amino acid sequence of SEQ ID NO: 253 and a light chain variable domain having at least 90% identity to the amino acid of SEQ ID NO: 279.

In one embodiment the antibody or an antigen-binding fragment thereof that neutralises SARS-CoV-2 with an IC50 of less than 2ug/ml comprises a heavy chain variable region sequence of QVQLVQSGPEVKKPGTSVKVSCKASGFTYTTSAVQWVRQARGQRLEWIGWIVAGS GNTNYAQKFQERVTITRDMSTGTAYMELSSLRSEDTAVYYCAAPGCNTTICPDGFDI WGRGTMVTVSS (SEQ ID NO: 254) and comprises a light chain variable region sequence of

(SEQ ID NO: 280)
EIVLTQSPGTQSLSPGERATLSCRASQSVSSSYLAWYQQKPGQAPRVLV
YGASSRARGIPDRFSGSGSGTDFTLSISRREREDCAVYYCQQYGSLPWT
FGQGTKVEIR.

In another embodiment, the antibody or antigen binding fragment thereof comprises a heavy chain variable domain having at least 90% identity to the amino acid sequence of SEQ ID NO: 254 and a light chain variable domain having at least 90% identity to the amino acid of SEQ ID NO: 280.

In one embodiment the antibody or an antigen-binding fragment thereof that neutralises SARS-CoV-2 with an IC50 of less than 2ug/ml comprises a heavy chain variable region sequence of LVQLVQSGAEVKKPGSSVKVSCKASGGTFSIYAISWVRQAPGQGLEWMGGIIPISGT ANYAQKFQDRVTITADESTSTAYMELSSLRSEDTAVYYCARLGRGDYDSSGYYKVY FDYWGQGTLVTVSS (SEQ ID NO: 255) and comprises a light chain variable region sequence of

(SEQ ID NO: 281)
DIQMTRSSFSLSASVGDRVTITCRASQNIGNYLNWYQQKPGIAPKLIIY
GASSLQSGVPSRFTGSGSGTDFTLTISSMQPEDFATYYCQKSYSGPYTF
GQGTKLEIK.

In another embodiment, the antibody or antigen binding fragment thereof comprises a heavy chain variable domain having at least 90% identity to the amino acid sequence of SEQ ID NO: 255 and a light chain variable domain having at least 90% identity to the amino acid of SEQ ID NO: 281

In one embodiment the antibody or an antigen-binding fragment thereof that neutralises SARS-CoV-2 with an IC50 of less than 2ug/ml comprises a heavy chain variable region sequence of EVQLVQSGVEVRKPGESVRISGKSSGYRFSNYWISWVRQMPGKGLEWMGTIDPSD YYTNYGPSFQGHVTRSGDKSISTAYRQWSSLKASDTAMYYCAKHKFFGELPIRGFD PWGQGTLVTVSS (SEQ ID NO: 256) and comprises a light chain variable region sequence of

(SEQ ID NO: 282)
EIVLTQSPGSLSLSPGERATLSCRASQSVSGSYIAWYQQKPGQAPRLLI
SGASNRATGIPDRFSGSGSGTDFTLTISRLEPEDFAVYSCQQYGSSPWT
FGQGTKVEIK.

In another embodiment, the antibody or antigen binding fragment thereof comprises a heavy chain variable domain having at least 90% identity to the amino acid sequence of SEQ ID NO: 256 and a light chain variable domain having at least 90% identity to the amino acid of SEQ ID NO: 282.

In one embodiment the antibody or an antigen-binding fragment thereof that neutralises SARS-CoV-2 with an IC50 of less than 2ug/ml comprises a heavy chain variable region sequence of VVQLVQSGAEVKKPGESLRISCKGSGYSFTTHWISWVRHMPGKGLEWMGRIDPSD SYTTYSPFFQGLVTISVDKSITTAYLHWSSLKASDTAIYYCARENFWSVYYTGIDYYM DVWGKGTTVTVSS (SEQ ID NO: 257) and comprises a light chain variable region sequence of

(SEQ ID NO: 283)
DIQMTQSPVSLSASIGDRVTFTCRASQGISNSFAWYQQKPGKAPKLLLY
AASRLESGVPSRFSGSGSGTDYTLTISSLQPEDFATYYCQQYYSTPYTF
GQGTKIEIK.

In another embodiment, the antibody or antigen binding fragment thereof comprises a heavy chain variable domain having at least 90% identity to the amino acid sequence of SEQ ID NO: 257 and a light chain variable domain having at least 90% identity to the amino acid of SEQ ID NO: 283.

In one embodiment the antibody or an antigen-binding fragment thereof that neutralises SARS-CoV-2 with an IC50 of less than 2ug/ml comprises a heavy chain variable region sequence of QVQLQQWGAGLLKPSETLSLTCAVYGEPLSGYFWTWIRQPPGKRLEWIGEISLRGS ANYNPSLKSRVTISIEVSKNQFSLKLTSVTAADMAVYYCSRGVVLNNVVWFDPWGQ GTLVTVSS (SEQ ID NO: 258) and comprises a light chain variable region sequence of

(SEQ ID NO: 284)
EIVLTQSPVTLSLSPGERATLSCRASQSVGSYLAWYQQKPGQAPRLLIY
GVSNRASGIPARFSGSGSGTDFTLTISGLEPEDFAVYYCQQRSIWLTFG
GGTKVEIK.

In another embodiment, the antibody or antigen binding fragment thereof comprises a heavy chain variable domain having at least 90% identity to the amino acid sequence of SEQ ID NO: 258 and a light chain variable domain having at least 90% identity to the amino acid of SEQ ID NO: 284.

In one embodiment the antibody or an antigen-binding fragment thereof that neutralises SARS-CoV-2 with an IC50 of less than 2ug/ml comprises a heavy chain variable region sequence of EVQLVESGGGVVQPGRSLRLSCAASGFTFSTYAMHWVRQAPGKGLEWVALISYDG STKYYADSVKGRFTISRDNSKITLYLHMNSLRAEDTAVYYCARDSEDCSSLSCYLDY WGQGTLVTVSS (SEQ ID NO: 259) and comprises a light chain variable region sequence of

(SEQ ID NO: 285)
DIVMTQSPDSLAVSLGERATINCKSSQSVLYSSNNKNYLGWYQQKPGQP
PKLLIYWASTRESGVPDRFSGSGSGTDFTLTISSLQAEDVAVYYCQQYY
STPFTFGPGTTVDIK.

In another embodiment, the antibody or antigen binding fragment thereof comprises a heavy chain variable domain having at least 90% identity to the amino acid sequence of SEQ ID NO: 259 and a light chain variable domain having at least 90% identity to the amino acid of SEQ ID NO: 285.

In one embodiment the antibody or an antigen-binding fragment thereof that neutralises SARS-CoV-2 with an IC50 of less than 2ug/ml comprises a heavy chain variable region sequence of EVQLVESGGGLVQPGGSLRLSCAASEFIVSRNYMSWVRQAPGKGLEWVSVIYSGG TTYYADSVKGRFTISRDSSKNTLYLQMNSLRAEDTAVYYCARDRGDYLFDYWGQGT LVTVSS (SEQ ID NO: 260) and comprises a light chain variable region sequence of

(SEQ ID NO: 286)
DIQMTQPPSPLFASVGDRVTITCRASQSISSWLAWYQQKPGKAPKLLIY
KASSLESGVPSRFSGSGSETEFTLTISSLQPDDFATYYCQQYNSYFPTF
GQGTKVEIK.

In another embodiment, the antibody or antigen binding fragment thereof comprises a heavy chain variable domain having at least 90% identity to the amino acid sequence of SEQ ID NO: 260 and a light chain variable domain having at least 90% identity to the amino acid of SEQ ID NO: 286.

In one embodiment the antibody or an antigen-binding fragment thereof that neutralises SARS-CoV-2 with an IC50 of less than 2ug/ml comprises a heavy chain variable region sequence of EVQLVESGGGLIQPGGSLRLSCVASGLTVSSNYMNWVRQTPGKGLEWVSVIYSGG STFYADSVKGRFTISRDNSKNTLFLQMNSLRAEDTAVYYCARVGGYCSSANCVSDV WGKGTTVTVSS (SEQ ID NO: 261) and comprises a light chain variable region sequence of

(SEQ ID NO: 287)
DIQMTQSPSSLSASVGDRVTITCQASQDIRNYLNWYQQKPGKAPKIMIY
DASILETGVPSRFSGSGSGTDFTFTISSLQPEDIATYYCQQYDNLPITF
GQGTQLEIK.

In another embodiment, the antibody or antigen binding fragment thereof comprises a heavy chain variable domain having at least 90% identity to the amino acid sequence of SEQ ID NO: 261 and a light chain variable domain having at least 90% identity to the amino acid of SEQ ID NO: 287.

In one embodiment the antibody or an antigen-binding fragment thereof that neutralises SARS-CoV-2 with an IC50 of less than 2ug/ml comprises a heavy chain variable region sequence of EVQLVESGGGLVQPGGSLRLSCAASGLTVSSNYMSWVRQAPGKGLEWVSVFYPG GSTYNADSVKGRFTISRDNSKNTLYLQMNSLRAEDTAVYYCARDAVYYGMDVWGQ GTTVTVSS (SEQ ID NO: 262) and comprises a light chain variable region sequence of

(SEQ ID NO: 288)
DIQMTQSPSSLSASVGDRVTITCRASQSISSYLNWYQQKPGKAPKLLIY
AASSLQSGVPSRFSGSGSGTDFTLTISSLQPEDFAIYYCQESYSTPGLF
TFGPGTKVDIK.

In another embodiment, the antibody or antigen binding fragment thereof comprises a heavy chain variable domain having at least 90% identity to the amino acid sequence of SEQ ID NO: 262 and a light chain variable domain having at least 90% identity to the amino acid of SEQ ID NO: 288.

In one embodiment the antibody or an antigen-binding fragment thereof that neutralises SARS-CoV-2 with an IC50 of less than 2ug/ml comprises a heavy chain variable region sequence of VVQLVESGGGLVKPGGSLRLSCAASGFTFSYAWMSWVRQAPGKGLEWVGRIKRK SDGGTTDYAAPVKGRFTISRDDSKNTLYLQMSSLKTEDTAVYYCTTDLCRSTSCEH DAFDIWGQGTMVTVSS (SEQ ID NO: 263) and comprises a light chain variable region sequence of

(SEQ ID NO: 289)
DIQMTQSPSSLSASVGDRVTVTCRASQSIRSYLNWYQQKPGKAPKLLIY
AASSLQSGVPSRFSGSGSGTDFTLTISSLQPEDFATYYCQQSYTTPAIT
FGQGTRVQIK.

In another embodiment, the antibody or antigen binding fragment thereof comprises a heavy chain variable domain having at least 90% identity to the amino acid sequence of SEQ ID NO: 263 and a light chain variable domain having at least 90% identity to the amino acid of SEQ ID NO: 289.

In one embodiment the antibody or an antigen-binding fragment thereof that neutralises SARS-CoV-2 with an IC50 of less than 2ug/ml comprises a heavy chain variable region sequence of EVQLVESGGGLIQPGGSLRLSCAASGFIVSSNYMSWVRQAPGKGLEWVAVLYPGG STFYADSVKGRFTISRDNSKNTLYLQMNSLRAEDTAVYYCARNIYDAFDIWGQGTMV TVSS (SEQ ID NO: 264) and comprises a light chain variable region sequence of

(SEQ ID NO: 290)
DIQLTQSPSSLSASVGDRVTITCRASQGIGSYLAWYQQKPGKAPKLLIY
AASTLQSGVPSRFSGSGSGTEFTLTISSLQPEDFATYYCQQLNSYPQGA
FGQGTKVDIK.

In another embodiment, the antibody or antigen binding fragment thereof comprises a heavy chain variable domain having at least 90% identity to the amino acid sequence of SEQ ID NO: 264 and a light chain variable domain having at least 90% identity to the amino acid of SEQ ID NO: 290.

In one embodiment the antibody or an antigen-binding fragment thereof that neutralises SARS-CoV-2 with an IC50 of less than 2ug/ml comprises a heavy chain variable region sequence of LVQLVQSGAEVKKPGSSVKVSCKASGGTFSIYAISWVRQAPGQGLEWMGGIIPISGT ANYAQKFQDRVTITADESTSTAYMELSSLRSEDTAVYYCARLGRGDYDSSGYYKVY FDYWGQGTLVTVSS (SEQ ID NO: 265) and comprises a light chain variable region sequence of

(SEQ ID NO: 291)
DIQMTRSSFSLSASVGDRVTITCRASQNIGNYLNWYQQKPGIAPKLIIY
GASSLQSGVPSRFTGSGSGTDFTLTISSMQPEDFATYYCQKSYSGPYTF
GQGTKLEIK.

In another embodiment, the antibody or antigen binding fragment thereof comprises a heavy chain variable domain having at least 90% identity to the amino acid sequence of SEQ ID NO: 265 and a light chain variable domain having at least 90% identity to the amino acid of SEQ ID NO: 291.

In one embodiment the antibody or an antigen-binding fragment thereof that neutralises SARS-CoV-2 with an IC50 of less than 2ug/ml comprises a heavy chain variable region sequence of EVQLVESGGGLVKPGGSLRLSCAASGFTFSDYYMSWIRQAPGKGPEWVSYISGGS SFTNYADSVQGRFTIYRDNAKNSMFLRMNSLRAEDTAVYYCARSPPTGDSSDWYD SPAYNNYYMDVWGKGTTVTVSS (SEQ ID NO: 266) and comprises a light chain variable region sequence of

(SEQ ID NO: 292)
EIVLTQSPVTLSLSPGERATLSCRASQSVSSSYLAWYQQKPGQAPRLLI
YGASSRATGIPDRFSGSGSGTDFTLTISRLEPEDFAVYYCQQYGTSPTV
TFGQGTRLEIK.

In another embodiment, the antibody or antigen binding fragment thereof comprises a heavy chain variable domain having at least 90% identity to the amino acid sequence of SEQ ID NO: 266 and a light chain variable domain having at least 90% identity to the amino acid of SEQ ID NO: 292.

In one embodiment the antibody or an antigen-binding fragment thereof that neutralises SARS-CoV-2 with an IC50 of less than 2ug/ml comprises a heavy chain variable region sequence of EVQLVESGGGLIQPGGSLRLSCAASGFTVSSNYMSWVRQAPGKGLKWVSVIYSGG STFYADSVKGRFTISRDDSKNTLFLQMNNLRAEDTAVYYCARLPYGDPAWGQGTLV TVSS (SEQ ID NO: 267) and comprises a light chain variable region sequence of

(SEQ ID NO: 293)
DIQMTQSPSSLSASVGDRVTITCLATQDIRNYLHWYQQKPGKAPKLIIY
DASHLETGVPSRFSGSGSGTDFTFTISSLQPEDIATYYCLQYDNLPLTF
GGGTKLEIK.

In another embodiment, the antibody or antigen binding fragment thereof comprises a heavy chain variable domain having at least 90% identity to the amino acid sequence of SEQ ID NO: 267 and a light chain variable domain having at least 90% identity to the amino acid of SEQ ID NO: 293.

In one embodiment the antibody or an antigen-binding fragment thereof that neutralises SARS-CoV-2 with an IC50 of less than 2ug/ml comprises a heavy chain variable region sequence of EVQLVESGGGVVQPGRSLRLSCAASGFTFSSFAMHWVRQAPGKGLEWVAVISYDG STKYSADSVKGRFTISRDNSKNTLYLQMNSLRAEDTAVYYCARDSEDCSSLSCYLDF WGQGTLVTVSS (SEQ ID NO: 268) and comprises a light chain variable region sequence of

(SEQ ID NO: 294)
DIVMTQSPVSLAVSLGERATINCKSSQSVLYRSNNKNYLAWYQQKPGQP
PKLLIYWASTRGSGVPDRFSGSGSGTDFTLTISSLQAEDVAVYYCQQYY
STPFTFGPGTKVDVK.

In another embodiment, the antibody or antigen binding fragment thereof comprises a heavy chain variable domain having at least 90% identity to the amino acid sequence of SEQ ID NO: 268 and a light chain variable domain having at least 90% identity to the amino acid of SEQ ID NO: 294.

In another embodiment, the antibody or antigen binding fragment thereof comprises a heavy chain variable domain having at least 80, 85, 90, 95, or 99% identity to the amino acid sequence recited and a light chain variable domain having at least 80, 85, 90, 95, or 99% identity to the amino acid sequence recited.

In one embodiment the antibody or an antigen-binding fragment thereof that neutralises SARS-CoV-2 with an IC50 of less than 2ug/ml comprises a HCDR1 of EFIVSRNY (SEQ ID NO: 18), a HCDR2 of IYSGGTT (SEQ ID NO: 44), a HCDR3 of ARDRGDYLFDY (SEQ ID NO: 70), a LCDR1 of QSISSW (SEQ ID NO: 96), a LCDR2 of KAS (SEQ ID NO: 122), and a LCDR3 of QQYNSYFPT (SEQ ID NO: 148).

In one embodiment the antibody or an antigen-binding fragment thereof that neutralises SARS-CoV-2 with an IC50 of less than 2ug/ml comprises a HCDR1 of GLTVSSNY (SEQ ID NO: 20), a HCDR2 of FYPGGST (SEQ ID NO: 46), a HCDR3 of ARDAVYYGMDV (SEQ ID NO: 72), a LCDR1 of QSISSY (SEQ ID NO: 98), a LCDR2 of AAS (SEQ ID NO: 124), and a LCDR3 of QESYSTPGLFT (SEQ ID NO: 150).

In one embodiment the antibody or an antigen-binding fragment thereof that neutralises SARS-CoV-2 with an IC50 of less than 2ug/ml comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134).

In one embodiment the antibody or an antigen-binding fragment thereof that neutralises SARS-CoV-2 with an IC50 of less than 2ug/ml comprises a HCDR1 of GLTVSSNY (SEQ ID NO: 19), a HCDR2 of IYSGGST (SEQ ID NO: 45), a HCDR3 of ARVGGYCSSANCVSDV (SEQ ID NO: 71), a LCDR1 of QDIRNY (SEQ ID NO: 97), a LCDR2 of DAS (SEQ ID NO: 123), and a LCDR3 of QQYDNLPIT (SEQ ID NO: 149).

In one embodiment the antibody or an antigen-binding fragment thereof that neutralises SARS-CoV-2 with an IC50 of less than 2ug/ml comprises a HCDR1 of GETLSGYY (SEQ ID NO: 11), a HCDR2 of ISLRGTA (SEQ ID NO: 37), a HCDR3 of VGGVVLDNVVWFDP (SEQ ID NO: 63), a LCDR1 of QSVGTY (SEQ ID NO: 89), a LCDR2 of NAS (SEQ ID NO: 115), and a LCDR3 of QQRSNWLT (SEQ ID NO: 141).

In one embodiment the antibody or an antigen-binding fragment thereof that neutralises SARS-CoV-2 with an IC50 of less than 2ug/ml comprises a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154).

In one embodiment the antibody or an antigen-binding fragment thereof that neutralises SARS-CoV-2 with an IC50 of less than 2ug/ml comprises a HCDR1 of GFTFSDYY (SEQ ID NO: 24), a HCDR2 of ISGGSSFT (SEQ ID NO: 50), and a HCDR3 of ARSPPTGDSSDWYDSPAYNNYYMDV (SEQ ID NO: 76).

In one embodiment the antibody or an antigen-binding fragment thereof that neutralises SARS-CoV-2 with an IC50 of less than 2ug/ml comprises a HCDR1 of GFTFSYAW (SEQ ID NO: 21), a HCDR2 of IKRKSDGGTT (SEQ ID NO: 47), a HCDR3 of TTDLCRSTSCEHDAFDI (SEQ ID NO: 73), a LCDR1 of QSIRSY (SEQ ID NO: 99), a LCDR2 of AAS (SEQ ID NO: 125), and a LCDR3 of QQSYTTPAIT (SEQ ID NO: 151).

In one embodiment the antibody or an antigen-binding fragment thereof that neutralises SARS-CoV-2 with an IC50 of less than 2ug/ml comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133).

In one embodiment the antibody or an antigen-binding fragment thereof that neutralises SARS-CoV-2 with an IC50 of less than 2ug/ml comprises a HCDR1 of GFNFYYSA (SEQ ID NO: 1), a HCDR2 of IVVGSGNT (SEQ ID NO: 27), a HCDR3 of AAPYCSGGSCHDGFDI (SEQ ID NO: 53), a LCDR1 of QSVRSNY (SEQ ID NO: 79), a LCDR2 of GAS (SEQ ID NO: 105), and a LCDR3 of QQYGSSPWT (SEQ ID NO: 131).

In one embodiment the antibody or an antigen-binding fragment thereof that neutralises SARS-CoV-2 with an IC50 of less than 2ug/ml comprises a HCDR1 of GFTFSNSA (SEQ ID NO: 2), a HCDR2 of IVVGSGNT (SEQ ID NO: 28), a HCDR3 of AAPYCGGDCNDGFDV (SEQ ID NO: 54), a LCDR1 of QSVRSSY (SEQ ID NO: 80), a LCDR2 of STS (SEQ ID NO: 106), and a LCDR3 of QQYGSSPWT (SEQ ID NO: 132).

In one embodiment the antibody or an antigen-binding fragment thereof that neutralises SARS-CoV-2 with an IC50 of less than 2ug/ml comprises a HCDR1 of GFTFDLSA (SEQ ID NO: 5), a HCDR2 of IAVGSGNT (SEQ ID NO: 31), a HCDR3 of AAPYCYSTSCADGFDI (SEQ ID NO: 57), a LCDR1 of QSVRSGY (SEQ ID NO: 83), a LCDR2 of GTS (SEQ ID NO: 109), and a LCDR3 of QQYGSSPWT (SEQ ID NO: 135).

In one embodiment the antibody or an antigen-binding fragment thereof that neutralises SARS-CoV-2 with an IC50 of less than 2ug/ml comprises a HCDR1 of GFTFSNAW (SEQ ID NO: 6), a HCDR2 of IKSKTDGGTT (SEQ ID NO: 32), a HCDR3 of TTDPGWWRIAVAGTNY (SEQ ID NO: 58), a LCDR1 of QSISSY (SEQ ID NO: 84), a LCDR2 of AAS (SEQ ID NO: 110), and a LCDR3 of QQSDSSPPT (SEQ ID NO: 136).

In one embodiment the antibody or an antigen-binding fragment thereof that neutralises SARS-CoV-2 with an IC50 of less than 2ug/ml comprises a HCDR1 of GFTFTLSA (SEQ ID NO: 7), a HCDR2 of IVPGSGNV (SEQ ID NO: 33), a HCDR3 of AAPYCNKTRCSDGFDI (SEQ ID NO: 59), a LCDR1 of QSVSSSY (SEQ ID NO: 85), a LCDR2 of GAS (SEQ ID NO: 111), and a LCDR3 of QQYGSSLFT (SEQ ID NO: 137).

29. An antibody or an antigen-binding fragment thereof according to claim 1 wherein the antibody or an antigen-binding fragment thereof comprises a HCDR1 of GFTFTSSA (SEQ ID NO: 8), a HCDR2 of IVVGSGNT (SEQ ID NO: 34), a HCDR3 of AAPNCNRTICADGFDI (SEQ ID NO: 60), a LCDR1 of QSVRSSY (SEQ ID NO: 86), a LCDR2 of ATS (SEQ ID NO: 112), and a LCDR3 of QQYGSSPWT (SEQ ID NO: 138).

In one embodiment the antibody or an antigen-binding fragment thereof that neutralises SARS-CoV-2 with an IC50 of less than 2ug/ml comprises a HCDR1 of GFTFTTSA (SEQ ID NO: 9), a HCDR2 of IAVGSGNT (SEQ ID NO: 35), a HCDR3 of AAPYCNTTSCDDGFDI (SEQ ID NO: 61), a LCDR1 of QSVRSNY (SEQ ID NO: 87), a LCDR2 of GAS (SEQ ID NO: 113), and a LCDR3 of QQYGSSPWT (SEQ ID NO: 139).

In one embodiment the antibody or an antigen-binding fragment thereof that neutralises SARS-CoV-2 with an IC50 of less than 2ug/ml comprises a HCDR1 of GFIFNRYW (SEQ ID NO: 10), a HCDR2 of IKQDGSEK (SEQ ID NO: 36), a HCDR3 of AADLGILWFGDLRKSEP (SEQ ID NO: 62), a LCDR1 of QGISNS (SEQ ID NO: 88), a LCDR2 of AAS (SEQ ID NO: 114), and a LCDR3 of QEYYSLRT (SEQ ID NO: 140).

In one embodiment the antibody or an antigen-binding fragment thereof that neutralises SARS-CoV-2 with an IC50 of less than 2ug/ml comprises a HCDR1 of GFTYTTSA (SEQ ID NO: 12), a HCDR2 of IVAGSGNT (SEQ ID NO: 38), a HCDR3 of AAPGCNTTICPDGFDI (SEQ ID NO: 64), a LCDR1 of QSVSSSY (SEQ ID NO: 90), a LCDR2 of GAS (SEQ ID NO: 116), and a LCDR3 of QQYGSLPWT (SEQ ID NO: 142).

In one embodiment the antibody or an antigen-binding fragment thereof that neutralises SARS-CoV-2 with an IC50 of less than 2ug/ml comprises a HCDR1 of GGTFSIYA (SEQ ID NO: 13), a HCDR2 of IIPISGTA (SEQ ID NO: 39), a HCDR3 of ARLGRGDYDSSGYYKVYFDY (SEQ ID NO: 65), a LCDR1 of QNIGNY (SEQ ID NO: 91), a LCDR2 of GAS (SEQ ID NO: 117), and a LCDR3 of QKSYSGPYT (SEQ ID NO: 143).

In one embodiment the antibody or an antigen-binding fragment thereof that neutralises SARS-CoV-2 with an IC50 of less than 2ug/ml comprises a HCDR1 of GYRFSNYW (SEQ ID NO: 14), a HCDR2 of IDPSDYYT (SEQ ID NO: 40), a HCDR3 of AKHKFFGELPIRGFDP (SEQ ID NO: 66), a LCDR1 of QSVSGSY (SEQ ID NO: 92), a LCDR2 of GAS (SEQ ID NO: 118), and a LCDR3 of QQYGSSPWT (SEQ ID NO: 144).

In one embodiment the antibody or an antigen-binding fragment thereof that neutralises SARS-CoV-2 with an IC50 of less than 2ug/ml comprises a HCDR1 of GYSFTTHW (SEQ ID NO: 15), a HCDR2 of IDPSDSYT (SEQ ID NO: 41), a HCDR3 of ARENFWSVYYTGIDYYMDV (SEQ ID NO: 67), a LCDR1 of QGISNS (SEQ ID NO: 93), a LCDR2 of AAS (SEQ ID NO: 119), and a LCDR3 of QQYYSTPYT (SEQ ID NO: 145)

In one embodiment the antibody or an antigen-binding fragment thereof that neutralises SARS-CoV-2 with an IC50 of less than 2ug/ml comprises a HCDR1 of GEPLSGYF (SEQ ID NO: 16), a HCDR2 of ISLRGSA (SEQ ID NO: 42), a HCDR3 of SRGVVLNNVVWFDP (SEQ ID NO: 68), a LCDR1 of QSVGSY (SEQ ID NO: 94), a LCDR2 of GVS (SEQ ID NO: 120), and a LCDR3 of QQRSIWLT (SEQ ID NO: 146).

In one embodiment the antibody or an antigen-binding fragment thereof that neutralises SARS-CoV-2 with an IC50 of less than 2ug/ml comprises a HCDR1 of GFTFSTYA (SEQ ID NO: 17), a HCDR2 of ISYDGSTK (SEQ ID NO: 43), a HCDR3 of ARDSEDCSSLSCYLDY (SEQ ID NO: 69), a LCDR1 of QSVLYSSNNKNY (SEQ ID NO: 95), a LCDR2 of WAS (SEQ ID NO: 121), and a LCDR3 of QQYYSTPFT (SEQ ID NO: 147).

In one embodiment the antibody or an antigen-binding fragment thereof that neutralises SARS-CoV-2 with an IC50 of less than 2ug/ml comprises a HCDR1 of GFIVSSNY (SEQ ID NO: 22), a HCDR2 of LYPGGST (SEQ ID NO: 48), a HCDR3 of ARNIYDAFDI (SEQ ID NO: 74), a LCDR1 of QGIGSY (SEQ ID NO: 100), a LCDR2 of AAS (SEQ ID NO: 126), and a LCDR3 of QQLNSYPQGA (SEQ ID NO: 152).

In one embodiment the antibody or an antigen-binding fragment thereof that neutralises SARS-CoV-2 with an IC50 of less than 2ug/ml comprises a HCDR1 of GFTVSSNY (SEQ ID NO: 25), a HCDR2 of IYSGGST (SEQ ID NO: 51), a HCDR3 of ARLPYGDPA (SEQ ID NO: 77), a LCDR1 of QDIRNY (SEQ ID NO: 103), a LCDR2 of DAS (SEQ ID NO: 129), and a LCDR3 of LQYDNLPLT (SEQ ID NO: 155).

In one embodiment the antibody or an antigen-binding fragment thereof that neutralises SARS-CoV-2 with an IC50 of less than 2ug/ml comprises a HCDR1 of GFTFSSFA (SEQ ID NO: 26), a HCDR2 of ISYDGSTK (SEQ ID NO: 52), a HCDR3 of ARDSEDCSSLSCYLDF (SEQ ID NO: 78), a LCDR1 of QSVLYRSNNKNY (SEQ ID NO: 104), a LCDR2 of WAS (SEQ ID NO: 130), and a LCDR3 of QQYYSTPFT (SEQ ID NO: 156).

In one embodiment the antibody or an antigen-binding fragment thereof that neutralises SARS-CoV-2 with an IC50 of less than 2ug/ml In one embodiment the antibody or an antigen-binding fragment thereof that neutralises SARS-CoV-2 with an IC50 of less than 2ug/ml comprises a heavy chain variable region sequence of QVQLVQSGPEVKKPGTSVKVSCKASGFNFYYSAVQWVRQARGQGLEWIGWIVVGS GNTNYAQKFQERVTITRDMSTSTAYMELSSLTSEDAAVYYCAAPYCSGGSCHDGFD IWGQGTMVTVSS(SEQ ID NO: 243) and comprises a light chain variable region sequence of

(SEQ ID NO: 268)
EIVLTQSPGSLSLSPGERATLSCRASQSVRSNYLAWYQQKPGQAPRVLI
YGASSRATGIPDRFSGSGSGTDFTLTISRLEPEDFAVYHCQQYGSSPWT
FGQGTKVEIK.

In another embodiment, the antibody or antigen binding fragment thereof comprises a heavy chain variable domain having at least 90% identity to the amino acid sequence of SEQ ID NO: 243 and a light chain variable domain having at least 90% identity to the amino acid of SEQ ID NO: 268.

In one embodiment the antibody or an antigen-binding fragment thereof that neutralises SARS-CoV-2 with an IC50 of less than 2ug/ml comprises a heavy chain variable region sequence of QVQLVQSGPEVKKPGTSVKVSCKASGFTFSNSAVQWVRQARGQRLEWIGWIVVGS GNTDYAQKFQERVTITRDMSTSTAYMELSSLRSEDTAVYFCAAPYCGGDCNDGFDV WGQGTMVTVSS(SEQ ID NO: 244) and comprises a light chain variable region sequence of

(SEQ ID NO: 269)
EIVLTQSPGSLSLSPGERATLSCRASQSVRSSYLAWYQQKLGQAPGLFI
YSTSRRATGIPDRFSGSGSGTDFTLTISRLEPEDFAVYYCQQYGSSPWT
FGQGTRVEIK.

In another embodiment, the antibody or antigen binding fragment thereof comprises a heavy chain variable domain having at least 90% identity to the amino acid sequence of SEQ ID NO: 244 and a light chain variable domain having at least 90% identity to the amino acid of SEQ ID NO: 269.

Comprises a heavy chain variable region sequence of LVQLVQSGAEVKKPGASVKISCKASGYTFTSYYMHWVRQAPGQGLEWMGIINPSG GGTSYAQKFQGRVTMTRDTSTSTVYMELTSLRSDDTAVYYCAKDRVTIFWGNGMD VWGQGTTVTVSS(SEQ ID NO: 245) and comprises a light chain variable region sequence of

(SEQ ID NO: 270)
DIVMTQSPVSLAVSLGERATIKCKSSQSVLYSSNNKNYLGWYQQKPGQP
LRLLIYWASTRESGVPDRFSGSGSGTDFTLTISSLQAEDVAVYYCHQYS
TTPLTFGGGTKVEIK.

In another embodiment, the antibody or antigen binding fragment thereof comprises a heavy chain variable domain having at least 90% identity to the amino acid sequence of SEQ ID NO: 245 and a light chain variable domain having at least 90% identity to the amino acid of SEQ ID NO: 270.

comprises a heavy chain variable region sequence of QVQLVQSGPEVKKPGTSVKVSCKASGFTFSSSAVQWVRQARGQRLEWIGWIVVGS GNANYAPRFQERVTITRDMSTNTAYMELSSLRSEDTAVYYCAAPNCSRTLCYDGFN MWGQGTMVTVSS(SEQ ID NO: 246) and comprises a light chain variable region sequence of

(SEQ ID NO: 271)
EIVLTQSPGSLSLSPGERATLSCRASQSVRSSYLGWYQQKPGQAPRLLI
YGASSRATGIPDRFSGSGSETDFTLTISRLEPEDFAVYYCQQYDSSPWT
FGQGTKVEIK.

In another embodiment, the antibody or antigen binding fragment thereof comprises a heavy chain variable domain having at least 90% identity to the amino acid sequence of SEQ ID NO: 246 and a light chain variable domain having at least 90% identity to the amino acid of SEQ ID NO: 271.

In one embodiment the antibody or an antigen-binding fragment thereof that neutralises SARS-CoV-2 with an IC50 of less than 2ug/ml comprises a heavy chain variable region sequence of QVQLAQSGPEVKKPGTSVKVSCKASGFTFDLSAVQWVRQARGQRLEWIGWIAVGS GNTDYAQKFQERVTLTRDMSTSTAYMELSSLRSEDTAVYYCAAPYCYSTSCADGFD IWGQGTMVTVSS(SEQ ID NO: 247) and comprises a light chain variable region sequence of

(SEQ ID NO: 272)
EIVLTQFPFSLSLSPGERATLSCRASQSVRSGYLAWYQQKPGQAPRLLI
DGTSSRATGIPDRISGSGFGTHFTLTISRVEPEDFAVYYCQQYGSSPWT
FGQGTKVEIK.

In another embodiment, the antibody or antigen binding fragment thereof comprises a heavy chain variable domain having at least 90% identity to the amino acid sequence of SEQ ID NO: 247 and a light chain variable domain having at least 90% identity to the amino acid of SEQ ID NO: 272.

In one embodiment the antibody or an antigen-binding fragment thereof that neutralises SARS-CoV-2 with an IC50 of less than 2ug/ml comprises a heavy chain variable region sequence of EVQLVESGGGLVKPGGSLRLSCAASGFTFSNAWMSWVRQAPGKGLEWVGHIKSKT DGGTTDYGAPVKGRFTISRDDSKNTLFLQMNSLKTEDTAVYYCTTDPGWWRIAVAG TNYWGQGTLVTVSS(SEQ ID NO: 248) and comprises a light chain variable region sequence of

(SEQ ID NO: 273)
DIQMTQSPFSLSASVGDRVTITCRASQSISSYLNWFQQKPGKAPKLLIY
AASSLQSGVPSRFSGSGSGTDFTLTISSLQPEDFATYYCQQSDSSPPTF
GQGTKVEFK.

In another embodiment, the antibody or antigen binding fragment thereof comprises a heavy chain variable domain having at least 90% identity to the amino acid sequence of SEQ ID NO: 248 and a light chain variable domain having at least 90% identity to the amino acid of SEQ ID NO: 273.

In one embodiment the antibody or an antigen-binding fragment thereof that neutralises SARS-CoV-2 with an IC50 of less than 2ug/ml comprises a heavy chain variable region sequence of QVQLVQSGPEVKKPGTSVKVSCKASGFTFTLSAVQWVRLARGQRLEWIGWIVPGS GNVNYAQKFQERVTITRDMSTNTDYMELSSLRSEDTAVYYCAAPYCNKTRCSDGFD IWGQGTMVTVSS(SEQ ID NO: 249) and comprises a light chain variable region sequence of

(SEQ ID NO: 274)
EIVLTQSPGSLSLSPGERATLSCRASQSVSSSYLAWYQQKPGQAPRLLI
YGASSRASGIPDRFSGSGSGTDFTLTISRLEPEDFAVYYCQQYGSSLFT
FGPGTKVDIK.

In another embodiment, the antibody or antigen binding fragment thereof comprises a heavy chain variable domain having at least 90% identity to the amino acid sequence of SEQ ID NO: 249 and a light chain variable domain having at least 90% identity to the amino acid of SEQ ID NO: 274.

In one embodiment the antibody or an antigen-binding fragment thereof that neutralises SARS-CoV-2 with an IC50 of less than 2ug/ml comprises a heavy chain variable region sequence of QVQLVQSGPEVKKPGTSVKVSCKASGFTFTSSAVQWVRQARGQRLEWIGWIVVGS GNTNYAQKFQERVTITRDMSTTTAYMEVSSLRSEDTAVYYCAAPNCNRTICADGFDI WGQGTIVTVSS(SEQ ID NO: 250) and comprises a light chain variable region sequence of

(SEQ ID NO: 275)
EIGLTQSPCTVSLSPWERTTLSCRASQSVRSSYLAWYQQKPGQAPRLLI
CATSSRATGIPDRFSGSGSGTDFTLTISRLEPEDFAVYYCQQYGSSPWT
FGQGTKVEIK.

In another embodiment, the antibody or antigen binding fragment thereof comprises a heavy chain variable domain having at least 90% identity to the amino acid sequence of SEQ ID NO: 250 and a light chain variable domain having at least 90% identity to the amino acid of SEQ ID NO: 275.

In one embodiment the antibody or an antigen-binding fragment thereof that neutralises SARS-CoV-2 with an IC50 of less than 2ug/ml comprises a heavy chain variable region sequence of KIQLVQSGPEVKKPGTSVKVSCKASGFTFTTSAVQWVRQARGQRLEWIGWIAVGSG NTEYEQKFQERVTITRDMSTSTAYMELNSLKSEDTAVYYCAAPYCNTTSCDDGFDI WGQGTMVTVSS(SEQ ID NO: 251) and comprises a light chain variable region sequence of

(SEQ ID NO: 276)
EIVLTQSPGTLSLSPGERATLSCRASQSVRSNYLAWYQQKPGQAPRVLI
YGASSRATGIPDRFSGSGSGTDFTLTISRLEPEDFAVYHCQQYGSSPWT
FGQGTKVEIK

In another embodiment, the antibody or antigen binding fragment thereof comprises a heavy chain variable domain having at least 90% identity to the amino acid sequence of SEQ ID NO: 251 and a light chain variable domain having at least 90% identity to the amino acid of SEQ ID NO: 276.

In one embodiment the antibody or an antigen-binding fragment thereof that neutralises SARS-CoV-2 with an IC50 of less than 2ug/ml comprises a heavy chain variable region sequence of EVQLVESGGGLVQPGGSLRLSCAASGFIFNRYWMTWVRQAPGKGLEWVANIKQDG SEKYYVDSVKGRFTISRDNAKNSLYLQMNSLRAEDTAVYYCAADLGILWFGDLRKSE PWGQGTLVTVSS(SEQ ID NO: 252) and comprises a light chain variable region sequence of

(SEQ ID NO: 277)
DIQMTQSPFSLSASVGDRVSITCRASQGISNSLAWYQQKPGKAPKLLLY
AASTLESGVPSRFSGSGSGTDFTLTISSLQPEDFATYYCQEYYSLRTFG
QGTKVEIK.

In another embodiment, the antibody or antigen binding fragment thereof comprises a heavy chain variable domain having at least 90% identity to the amino acid sequence of SEQ ID NO: 252 and a light chain variable domain having at least 90% identity to the amino acid of SEQ ID NO: 277.

In one embodiment the antibody or an antigen-binding fragment thereof that neutralises SARS-CoV-2 with an IC50 of less than 2ug/ml comprises a heavy chain variable region sequence of QVQLQQWGAGLLKPSETLSLTCVVYGETLSGYYWTWIRQSPGKGLEWIGEISLRGT ANYNPSLKSRVTLSVEASKNQFSLKMTSVTAADTAVYYCVGGVVLDNVVWFDPWG QGIPVTVSL(SEQ ID NO: 253) and comprises a light chain variable region sequence of

(SEQ ID NO: 278)
ESVLTQSPVSLSLSPGERATLSCRASQSVGTYLAWYQHRPGQAPRLLIYN
ASKRATGIPARFSGSGSGTDFTLTISSLEPEDLAVYFCQQRSNWLTFGGG
TKVEIK.

In another embodiment, the antibody or antigen binding fragment thereof comprises a heavy chain variable domain having at least 90% identity to the amino acid sequence of SEQ ID NO: 253 and a light chain variable domain having at least 90% identity to the amino acid of SEQ ID NO: 278.

In one embodiment the antibody or an antigen-binding fragment thereof that neutralises SARS-CoV-2 with an IC50 of less than 2ug/ml comprises a heavy chain variable region sequence of QVQLVQSGPEVKKPGTSVKVSCKASGFTYTTSAVQWVRQARGQRLEWIGWIVAGS GNTNYAQKFQERVTITRDMSTGTAYMELSSLRSEDTAVYYCAAPGCNTTICPDGFDI WGRGTMVTVSS(SEQ ID NO: 254) and comprises a light chain variable region sequence of

(SEQ ID NO: 279)
EIVLTQSPGTQSLSPGERATLSCRASQSVSSSYLAWYQQKPGQAPRVLVY
GASSRARGIPDRFSGSGSGTDFTLSISRREREDCAVYYCQQYGSLPWTFG
QGTKVEIR.

In another embodiment, the antibody or antigen binding fragment thereof comprises a heavy chain variable domain having at least 90% identity to the amino acid sequence of SEQ ID NO: 254 and a light chain variable domain having at least 90% identity to the amino acid of SEQ ID NO: 279.

In one embodiment the antibody or an antigen-binding fragment thereof that neutralises SARS-CoV-2 with an IC50 of less than 2ug/ml comprises a heavy chain variable region sequence of LVQLVQSGAEVKKPGSSVKVSCKASGGTFSIYAISWVRQAPGQGLEWMGGIIPISGT ANYAQKFQDRVTITADESTSTAYMELSSLRSEDTAVYYCARLGRGDYDSSGYYKVY FDYWGQGTLVTVSS(SEQ ID NO: 255) and comprises a light chain variable region sequence of

(SEQ ID NO: 280)
DIQMTRSSFSLSASVGDRVTITCRASQNIGNYLNWYQQKPGIAPKLIIYG
ASSLQSGVPSRFTGSGSGTDFTLTISSMQPEDFATYYCQKSYSGPYTFGQ
GTKLEIK.

In another embodiment, the antibody or antigen binding fragment thereof comprises a heavy chain variable domain having at least 90% identity to the amino acid sequence of SEQ ID NO: 255 and a light chain variable domain having at least 90% identity to the amino acid of SEQ ID NO: 280.

In one embodiment the antibody or an antigen-binding fragment thereof that neutralises SARS-CoV-2 with an IC50 of less than 2ug/ml comprises a heavy chain variable region sequence of EVQLVQSGVEVRKPGESVRISGKSSGYRFSNYWISWVRQMPGKGLEWMGTIDPSD YYTNYGPSFQGHVTRSGDKSISTAYRQWSSLKASDTAMYYCAKHKFFGELPIRGFD PWGQGTLVTVSS(SEQ ID NO: 256) and comprises a light chain variable region sequence of

(SEQ ID NO: 281)
EIVLTQSPGSLSLSPGERATLSCRASQSVSGSYIAWYQQKPGQAPRLLIS
GASNRATGIPDRFSGSGSGTDFTLTISRLEPEDFAVYSCQQYGSSPWTFG
QGTKVEIK.

In another embodiment, the antibody or antigen binding fragment thereof comprises a heavy chain variable domain having at least 90% identity to the amino acid sequence of SEQ ID NO: 256 and a light chain variable domain having at least 90% identity to the amino acid of SEQ ID NO: 281

In one embodiment the antibody or an antigen-binding fragment thereof that neutralises SARS-CoV-2 with an IC50 of less than 2ug/ml comprises a heavy chain variable region sequence of VVQLVQSGAEVKKPGESLRISCKGSGYSFTTHWISWVRHMPGKGLEWMGRIDPSD SYTTYSPFFQGLVTISVDKSITTAYLHWSSLKASDTAIYYCARENFWSVYYTGIDYYM DVWGKGTTVTVSS(SEQ ID NO: 257) and comprises a light chain variable region sequence of

(SEQ ID NO: 282)
DIQMTQSPVSLSASIGDRVTFTCRASQGISNSFAWYQQKPGKAPKLLLYA
ASRLESGVPSRFSGSGSGTDYTLTISSLQPEDFATYYCQQYYSTPYTFGQ
GTKIEIK.

In another embodiment, the antibody or antigen binding fragment thereof comprises a heavy chain variable domain having at least 90% identity to the amino acid sequence of SEQ ID NO: 257 and a light chain variable domain having at least 90% identity to the amino acid of SEQ ID NO: 282.

In one embodiment the antibody or an antigen-binding fragment thereof that neutralises SARS-CoV-2 with an IC50 of less than 2ug/ml comprises a heavy chain variable region sequence of QVQLQQWGAGLLKPSETLSLTCAVYGEPLSGYFWTWIRQPPGKRLEWIGEISLRGS ANYNPSLKSRVTISIEVSKNQFSLKLTSVTAADMAVYYCSRGVVLNNVVWFDPWGQ GTLVTVSS(SEQ ID NO: 258) and comprises a light chain variable region sequence of

(SEQ ID NO: 283)
EIVLTQSPVTLSLSPGERATLSCRASQSVGSYLAWYQQKPGQAPRLLIYG
VSNRASGIPARFSGSGSGTDFTLTISGLEPEDFAVYYCQQRSIWLTFGGG
TKVEIK.

In another embodiment, the antibody or antigen binding fragment thereof comprises a heavy chain variable domain having at least 90% identity to the amino acid sequence of SEQ ID NO: 258 and a light chain variable domain having at least 90% identity to the amino acid of SEQ ID NO: 283.

In one embodiment the antibody or an antigen-binding fragment thereof that neutralises SARS-CoV-2 with an IC50 of less than 2ug/ml comprises a heavy chain variable region sequence of EVQLVESGGGVVQPGRSLRLSCAASGFTFSTYAMHWVRQAPGKGLEWVALISYDG STKYYADSVKGRFTISRDNSKITLYLHMNSLRAEDTAVYYCARDSEDCSSLSCYLDY WGQGTLVTVSS(SEQ ID NO: 259) and comprises a light chain variable region sequence of

(SEQ ID NO: 284)
DIVMTQSPDSLAVSLGERATINCKSSQSVLYSSNNKNYLGWYQQKPGQPP
KLLIYWASTRESGVPDRFSGSGSGTDFTLTISSLQAEDVAVYYCQQYYST
PFTFGPGTTVDIK.

In another embodiment, the antibody or antigen binding fragment thereof comprises a heavy chain variable domain having at least 90% identity to the amino acid sequence of SEQ ID NO: 259 and a light chain variable domain having at least 90% identity to the amino acid of SEQ ID NO: 284.

In one embodiment the antibody or an antigen-binding fragment thereof that neutralises SARS-CoV-2 with an IC50 of less than 2ug/ml comprises a heavy chain variable region sequence of EVQLVESGGGLVQPGGSLRLSCAASEFIVSRNYMSWVRQAPGKGLEWVSVIYSGG TTYYADSVKGRFTISRDSSKNTLYLQMNSLRAEDTAVYYCARDRGDYLFDYWGQGT LVTVSS(SEQ ID NO: 260) and comprises a light chain variable region sequence of

(SEQ ID NO: 285)
DIQMTQPPSPLFASVGDRVTITCRASQSISSWLAWYQQKPGKAPKLLIYK
ASSLESGVPSRFSGSGSETEFTLTISSLQPDDFATYYCQQYNSYFPTFGQ
GTKVEIK.

In another embodiment, the antibody or antigen binding fragment thereof comprises a heavy chain variable domain having at least 90% identity to the amino acid sequence of SEQ ID NO: 260 and a light chain variable domain having at least 90% identity to the amino acid of SEQ ID NO: 285.

In one embodiment the antibody or an antigen-binding fragment thereof that neutralises SARS-CoV-2 with an IC50 of less than 2ug/ml comprises a heavy chain variable region sequence of EVQLVESGGGLIQPGGSLRLSCVASGLTVSSNYMNWVRQTPGKGLEWVSVIYSGG STFYADSVKGRFTISRDNSKNTLFLQMNSLRAEDTAVYYCARVGGYCSSANCVSDV WGKGTTVTVSS(SEQ ID NO: 261) and comprises a light chain variable region sequence of

(SEQ ID NO: 286)
DIQMTQSPSSLSASVGDRVTITCQASQDIRNYLNWYQQKPGKAPKIMIYD
ASILETGVPSRFSGSGSGTDFTFTISSLQPEDIATYYCQQYDNLPITFGQ
GTQLEIK.

In another embodiment, the antibody or antigen binding fragment thereof comprises a heavy chain variable domain having at least 90% identity to the amino acid sequence of SEQ ID NO: 261 and a light chain variable domain having at least 90% identity to the amino acid of SEQ ID NO: 286.

In one embodiment the antibody or an antigen-binding fragment thereof that neutralises SARS-CoV-2 with an IC50 of less than 2ug/ml comprises a heavy chain variable region sequence of EVQLVESGGGLVQPGGSLRLSCAASGLTVSSNYMSWVRQAPGKGLEWVSVFYPG GSTYNADSVKGRFTISRDNSKNTLYLQMNSLRAEDTAVYYCARDAVYYGMDVWGQ GTTVTVSS(SEQ ID NO: 262) and comprises a light chain variable region sequence of

(SEQ ID NO: 287)
DIQMTQSPSSLSASVGDRVTITCRASQSISSYLNWYQQKPGKAPKLLIYA
ASSLQSGVPSRFSGSGSGTDFTLTISSLQPEDFAIYYCQESYSTPGLFTF
GPGTKVDIK.

In another embodiment, the antibody or antigen binding fragment thereof comprises a heavy chain variable domain having at least 90% identity to the amino acid sequence of SEQ ID NO: 262 and a light chain variable domain having at least 90% identity to the amino acid of SEQ ID NO: 287.

In one embodiment the antibody or an antigen-binding fragment thereof that neutralises SARS-CoV-2 with an IC50 of less than 2ug/ml comprises a heavy chain variable region sequence of VVQLVESGGGLVKPGGSLRLSCAASGFTFSYAWMSWVRQAPGKGLEWVGRIKRK SDGGTTDYAAPVKGRFTISRDDSKNTLYLQMSSLKTEDTAVYYCTTDLCRSTSCEH DAFDIWGQGTMVTVSS(SEQ ID NO: 263) and comprises a light chain variable region sequence of

(SEQ ID NO: 288)
DIQMTQSPSSLSASVGDRVTVTCRASQSIRSYLNWYQQKPGKAPKLLIYA
ASSLQSGVPSRFSGSGSGTDFTLTISSLQPEDFATYYCQQSYTTPAITFG
QGTRVQIK.

In another embodiment, the antibody or antigen binding fragment thereof comprises a heavy chain variable domain having at least 90% identity to the amino acid sequence of SEQ ID NO: 263 and a light chain variable domain having at least 90% identity to the amino acid of SEQ ID NO: 288.

In one embodiment the antibody or an antigen-binding fragment thereof that neutralises SARS-CoV-2 with an IC50 of less than 2ug/ml comprises a heavy chain variable region sequence of EVQLVESGGGLIQPGGSLRLSCAASGFIVSSNYMSWVRQAPGKGLEWVAVLYPGG STFYADSVKGRFTISRDNSKNTLYLQMNSLRAEDTAVYYCARNIYDAFDIWGQGTMV TVSS(SEQ ID NO: 264) and comprises a light chain variable region sequence of

(SEQ ID NO: 289)
DIQLTQSPSSLSASVGDRVTITCRASQGIGSYLAWYQQKPGKAPKLLIYA
ASTLQSGVPSRFSGSGSGTEFTLTISSLQPEDFATYYCQQLNSYPQGAFG
QGTKVDIK.

In another embodiment, the antibody or antigen binding fragment thereof comprises a heavy chain variable domain having at least 90% identity to the amino acid sequence of SEQ ID NO: 264 and a light chain variable domain having at least 90% identity to the amino acid of SEQ ID NO: 289.

In one embodiment the antibody or an antigen-binding fragment thereof that neutralises SARS-CoV-2 with an IC50 of less than 2ug/ml comprises a heavy chain variable region sequence of EVQLVESGGGLVKPGGSLRLSCAASGFTFSDYYMSWIRQAPGKGPEWVSYISGGS SFTNYADSVQGRFTIYRDNAKNSMFLRMNSLRAEDTAVYYCARSPPTGDSSDWYD SPAYNNYYMDVWGKGTTVTVSS(SEQ ID NO: 265) and comprises a light chain variable region sequence of

(SEQ ID NO: 290)
EIVLTQSPVTLSLSPGERATLSCRASQSVSSSYLAWYQQKPGQAPRLLIY
GASSRATGIPDRFSGSGSGTDFTLTISRLEPEDFAVYYCQQYGTSPTVTF
GQGTRLEIK.

In another embodiment, the antibody or antigen binding fragment thereof comprises a heavy chain variable domain having at least 90% identity to the amino acid sequence of SEQ ID NO: 265 and a light chain variable domain having at least 90% identity to the amino acid of SEQ ID NO: 290.

In one embodiment the antibody or an antigen-binding fragment thereof that neutralises SARS-CoV-2 with an IC50 of less than 2ug/ml comprises a heavy chain variable region sequence of EVQLVESGGGLIQPGGSLRLSCAASGFTVSSNYMSWVRQAPGKGLKWVSVIYSGG STFYADSVKGRFTISRDDSKNTLFLQMNNLRAEDTAVYYCARLPYGDPAWGQGTLV TVSS(SEQ ID NO: 266) and comprises a light chain variable region sequence of

(SEQ ID NO: 291)
DIQMTQSPSSLSASVGDRVTITCLATQDIRNYLHWYQQKPGKAPKLIIYD
ASHLETGVPSRFSGSGSGTDFTFTISSLQPEDIATYYCLQYDNLPLTFGG
GTKLEIK.

In another embodiment, the antibody or antigen binding fragment thereof comprises a heavy chain variable domain having at least 90% identity to the amino acid sequence of SEQ ID NO: 266 and a light chain variable domain having at least 90% identity to the amino acid of SEQ ID NO: 291.

In one embodiment the antibody or an antigen-binding fragment thereof that neutralises SARS-CoV-2 with an IC50 of less than 2ug/ml comprises a heavy chain variable region sequence of EVQLVESGGGVVQPGRSLRLSCAASGFTFSSFAMHWVRQAPGKGLEWVAVISYDG STKYSADSVKGRFTISRDNSKNTLYLQMNSLRAEDTAVYYCARDSEDCSSLSCYLDF WGQGTLVTVSS(SEQ ID NO: 267) and comprises a light chain variable region sequence of

(SEQ ID NO: 292)
DIVMTQSPVSLAVSLGERATINCKSSQSVLYRSNNKNYLAWYQQKPGQPP
KLLIYWASTRGSGVPDRFSGSGSGTDFTLTISSLQAEDVAVYYCQQYYST
PFTFGPGTKVDVK

In another embodiment, the antibody or antigen binding fragment thereof comprises a heavy chain variable domain having at least 90% identity to the amino acid sequence of SEQ ID NO: 267 and a light chain variable domain having at least 90% identity to the amino acid of SEQ ID NO: 292

The present inventors have also demonstrated highly potent mAbs bound one of up to 6 putative epitope clusters localised across all faces of the RBD, which except for PDI 215, efficiently blocked engagement of the cellular receptor ACE2.

Alternative epitope cluster schemes have been proposed in the field, including in Hastie et al. (2021) Science 374(6566): 472-478, and Barnes, et al. (2020) Nature 588, 682-687.

Table 2 shows the SARS-CoV-2 spike receptor-binding domain (RBD) residues that comprise epitope cluster 1 (PDI 37, PDI 42, PDI 222, PDI, 231), SARS-CoV-2 spike receptor-binding domain (RBD) residues that comprise epitope cluster 2 (PDI 210); SARS-CoV-2 spike receptor-binding domain (RBD) residues that comprise epitope cluster 4 (PDI 215); SARS-CoV-2 spike receptor-binding domain (RBD) residues that comprise epitope cluster 5 (PDI 93); and SARS-CoV-2 spike receptor-binding domain (RBD) residues that comprise epitope cluster 6 (PDI 96).

In one embodiment the present invention provides an antibody or an antigen-binding fragment thereof as described herein that binds to epitope cluster 1 of the SARS-CoV-2 spike receptor-binding domain (RBD).

As used herein, the term epitope cluster 1 refers to an epitope of the SARS-CoV-2 spike receptor-binding domain comprising one or more residues selected from the group consisting of the ACE2 footprint overlapping residues 487, 475, 486, 489, 456, 455, 417, 453, 493, 505, 501, 502, 500, 498, and 496 of SARS-CoV-2 isolate WHU1 spike RBD, or an equivalent position of a different SARS-CoV-2 isolate spike RBD.

In one embodiment, the epitope cluster 1 comprises the ACE2 footprint overlapping residues 487, 475, 486, 489, 456, 455, 417, 493, 453, 505, 501, 502, 500, 498, and 501 of SARS-CoV-2 isolate WHU1 spike RBD, or an equivalent position of a different SARS-CoV-2 isolate spike RBD.

In another embodiment, the epitope cluster 1 comprises the ACE2 footprint overlapping residues 487, 475, 486, 489, 456, 455, 417, 453, 493, 505, 502, 501, 496, and 498 of SARS-CoV-2 isolate WHU1 spike RBD, or an equivalent position of a different SARS-CoV-2 isolate spike RBD.

In another embodiment, the epitope cluster 1 comprises the ACE2 footprint overlapping residues 487, 475, 486, 456, 489, 455, and 493 of SARS-CoV-2 isolate WHU1 spike RBD, or an equivalent position of a different SARS-CoV-2 isolate spike RBD.

In one embodiment, the epitope cluster 1 consists of the ACE2 footprint overlapping residues 487, 475, 486, 489, 456, 455, 417, 493, 453, 505, 501, 502, 500, 498, and 501 of SARS-CoV-2 isolate WHU1 spike RBD, or an equivalent position of a different SARS-CoV-2 isolate spike RBD.

In another embodiment, the epitope cluster 1 consists of the ACE2 footprint overlapping residues 487, 475, 486, 489, 456, 455, 417, 453, 493, 505, 502, 501, 496, and 498 of SARS-CoV-2 isolate WHU1 spike RBD, or an equivalent position of a different SARS-CoV-2 isolate spike RBD.

In another embodiment, the epitope cluster 1 consists of the ACE2 footprint overlapping residues 487, 475, 486, 456, 489, 455, and 493 of SARS-CoV-2 isolate WHU1 spike RBD, or an equivalent position of a different SARS-CoV-2 isolate spike RBD.

In another embodiment, the epitope cluster 1 comprises the ACE2 footprint overlapping residues 487, 475, 486, 489, 456, 455, 417, 453, 493, 505, 502, 498, 501, and 496 of SARS-CoV-2 isolate WHU1 spike RBD, or an equivalent position of a different SARS-CoV-2 isolate spike RBD.

In one embodiment the present invention provides an antibody or an antigen-binding fragment thereof as described herein that binds to epitope cluster 1 of the SARS-CoV-2 spike RBD comprises a HCDR1 of EFIVSRNY (SEQ ID NO: 18), a HCDR2 of IYSGGTT (SEQ ID NO: 44), a HCDR3 of ARDRGDYLFDY (SEQ ID NO: 70), a LCDR1 of QSISSW (SEQ ID NO: 96), a LCDR2 of KAS (SEQ ID NO: 122), and a LCDR3 of QQYNSYFPT (SEQ ID NO: 148).

In one embodiment the present invention provides an antibody or an antigen-binding fragment thereof as described herein that binds to epitope cluster 1 of the SARS-CoV-2 spike RBD comprises a HCDR1 of GLTVSSNY (SEQ ID NO: 20), a HCDR2 of FYPGGST (SEQ ID NO: 46), a HCDR3 of ARDAVYYGMDV (SEQ ID NO: 72), a LCDR1 of QSISSY (SEQ ID NO: 98), a LCDR2 of AAS (SEQ ID NO: 124), and a LCDR3 of QESYSTPGLFT (SEQ ID NO: 150).

In one embodiment the present invention provides an antibody or an antigen-binding fragment thereof as described herein that binds to epitope cluster 1 of the SARS-CoV-2 spike RBD comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134).

In one embodiment the present invention provides an antibody or an antigen-binding fragment thereof as described herein that binds to epitope cluster 1 of the SARS-CoV-2 spike RBD comprises a HCDR1 of GLTVSSNY (SEQ ID NO: 19), a HCDR2 of IYSGGST (SEQ ID NO: 45), a HCDR3 of ARVGGYCSSANCVSDV (SEQ ID NO: 71), a LCDR1 of QDIRNY (SEQ ID NO: 97), a LCDR2 of DAS (SEQ ID NO: 123), and a LCDR3 of QQYDNLPIT (SEQ ID NO: 149).

In one embodiment the present invention provides an antibody or an antigen-binding fragment thereof as described herein that binds to epitope cluster 2 of the SARS-CoV-2 spike receptor-binding domain (RBD).

As used herein, the term epitope cluster 2 refers to an epitope of the SARS-CoV-2 spike receptor-binding domain comprising one or more residues selected from the group consisting of the ACE2 footprint overlapping residues 487, 475, 486, 489, 456, 455, 453, 493, 505, 501, 498, 449 and 496 of SARS-CoV-2 isolate WHU1 spike RBD, or an equivalent position of a different SARS-CoV-2 isolate spike RBD.

In one embodiment, the epitope cluster 2 comprises the ACE2 footprint overlapping residues 487, 475, 486, 489, 456, 455, 453, 493, 505, 501, 498, 449 and of SARS-CoV-2 isolate WHU1 spike RBD, or an equivalent position of a different SARS-CoV-2 isolate spike RBD.

In one embodiment the present invention provides an antibody or an antigen-binding fragment thereof as described herein that binds to epitope cluster 2 of the SARS-CoV-2 spike RBD comprises a HCDR1 of GETLSGYY (SEQ ID NO: 11), a HCDR2 of ISLRGTA (SEQ ID NO: 37), a HCDR3 of VGGVVLDNVVWFDP (SEQ ID NO: 63), a LCDR1 of QSVGTY (SEQ ID NO: 89), a LCDR2 of NAS (SEQ ID NO: 115), and a LCDR3 of QQRSNWLT (SEQ ID NO: 141).

In one embodiment the present invention provides an antibody or an antigen-binding fragment thereof as described herein that binds to epitope cluster 3 of the SARS-CoV-2 spike receptor-binding domain (RBD).

As used herein, the term epitope cluster 3 refers to an epitope of the SARS-CoV-2 spike receptor-binding domain comprising one or more residues selected from the group consisting of the ACE2 footprint overlapping residues 487, 475, 486, 489, 456, 455, 493, and 449 of SARS-CoV-2 isolate WHU1 spike RBD, or an equivalent position of a different SARS-CoV-2 isolate spike RBD.

In one embodiment, the epitope cluster 3 comprises the ACE2 footprint overlapping residues 487, 475, 486, 489, 456, 455, 493, and 449 of SARS-CoV-2 isolate WHU1 spike RBD, or an equivalent position of a different SARS-CoV-2 isolate spike RBD.

In one embodiment the present invention provides an antibody or an antigen-binding fragment thereof as described herein that binds to epitope cluster 4 of the SARS-CoV-2 spike receptor-binding domain (RBD).

As used herein, the term epitope cluster 4 refers to an epitope of the SARS-CoV-2 spike receptor-binding domain comprising the ACE2 footprint overlapping residues 449, but not the ACE2 footprint overlapping residues 487, 475, 486, 489, 456, 455, 417, 453, 493, 496, 505, 501, 502, 500, 498, or 446 of SARS-CoV-2 isolate WHU1 spike RBD, or an equivalent position of a different SARS-CoV-2 isolate spike RBD.

In one embodiment, the epitope cluster 4 consists of the ACE2 footprint overlapping residue 449 of SARS-CoV-2 isolate WHU1 spike RBD, or an equivalent position of a different SARS-CoV-2 isolate spike RBD.

In one embodiment the present invention provides an antibody or an antigen-binding fragment thereof as described herein that binds to epitope cluster 4 of the SARS-CoV-2 spike RBD comprises a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154).

(SEQ ID NO: 154)
QQYGTSPTVT.

In one embodiment the present invention provides an antibody or an antigen-binding fragment thereof as described herein that binds to epitope cluster 4 of the SARS-CoV-2 spike RBD comprises a HCDR1 of GFTFSDYY (SEQ ID NO: 24), a HCDR2 of ISGGSSFT (SEQ ID NO: 50), and a HCDR3 of

(SEQ ID NO: 76)
ARSPPTGDSSDWYDSPAYNNYYMDV.

In one embodiment the present invention provides an antibody or an antigen-binding fragment thereof as described herein that binds to epitope cluster 5 of the SARS-CoV-2 spike receptor-binding domain (RBD).

As used herein, the term epitope cluster 5 refers to an epitope of the SARS-CoV-2 spike receptor-binding domain comprising the ACE2 footprint overlapping residues 493, 449 and 446, but not the ACE2 footprint overlapping residues 487, 475, 486, 489, 456, 455, 417, 453, 496, 505, 501, 502, 500, or 498, of SARS-CoV-2 isolate WHU1 spike RBD, or an equivalent position of a different SARS-CoV-2 isolate RBD.

In one embodiment, the epitope cluster 5 consists of the ACE2 footprint overlapping residues 446, 449 and 493 of SARS-CoV-2 isolate WHU1 spike RBD, or an equivalent position of a different SARS-CoV-2 isolate spike RBD.

In one embodiment the present invention provides an antibody or an antigen-binding fragment thereof as described herein that binds to epitope cluster 5 of the SARS-CoV-2 spike RBD comprises a HCDR1 of GFTFSYAW (SEQ ID NO: 21), a HCDR2 of IKRKSDGGTT (SEQ ID NO: 47), a HCDR3 of TTDLCRSTSCEHDAFDI (SEQ ID NO: 73), a LCDR1 of QSIRSY (SEQ ID NO: 99), a LCDR2 of AAS (SEQ ID NO: 125), and a LCDR3 of QQSYTTPAIT (SEQ ID NO: 151).

In one embodiment the present invention provides an antibody or an antigen-binding fragment thereof as described herein that binds to epitope cluster 6 of the SARS-CoV-2 spike receptor-binding domain (RBD).

As used herein, the term epitope cluster 6 refers to an epitope of the SARS-CoV-2 spike receptor-binding domain comprising the ACE2 footprint overlapping residues 446 and 500, but not the ACE2 footprint overlapping residues 487, 475, 486, 489, 456, 455, 417, 453, 493, 496, 505, 501, 502, 498, or 449 of SARS-CoV-2 isolate WHU1 spike RBD, or an equivalent position of a different SARS-CoV-2 isolate spike RBD.

In one embodiment, the epitope cluster 6 consists of the ACE2 footprint overlapping residues 446 and 500 of SARS-CoV-2 isolate WHU1 spike RBD, or an equivalent position of a different SARS-CoV-2 isolate spike RBD.

In one embodiment the present invention provides an antibody or an antigen-binding fragment thereof as described herein that binds to epitope cluster 6 of the SARS-CoV-2 spike RBD comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133)

In one embodiment the antibody or an antigen-binding fragment thereof neutralises SARS-CoV-2 with an IC50 of less than 2ug/ml.

In another embodiment, the present invention provides an antibody or antigen binding fragment thereof that neutralises SARS-CoV-2 with an IC50 of less than 10ug/ml.

In another embodiment, the present invention provides an antibody or antigen binding fragment thereof that neutralises SARS-CoV-2 with an IC50 of less than 50ug/ml.

In another embodiment, the present invention provides an antibody or antigen binding fragment thereof that neutralises SARS-CoV-2 with an IC50 of less than 100ug/ml.

In another embodiment, the present invention provides an antibody or antigen binding fragment thereof that neutralises SARS-CoV-2 with an IC50 of less than 200ug/ml.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of QVQLVQSGPEVKKPGTSVKVSCKASGFTFTSSAVQWVRQARGQRLEWIGWIVVGS GNTEYAQKFQERVTITRDMSTSTAYMELSSLRSEDTAVYYCAANNCGRTTCSDAFDI WGQGTMVTLSS (SEQ ID NO: 157) and comprises a light chain variable region sequence of

(SEQ ID NO: 200)
EIVLTQSPGTLSLSSGERATLSGRASQSVSSSYLAWYQQKPGQAPRLLM
YGASSRATGIPDRFSGSGSGTDFTLTISRLEREDFAVYYCQQFRSSPWT
FGQGTKVEIK.

In another embodiment, the antibody or antigen binding fragment thereof comprises a heavy chain variable domain having at least 90% identity to the amino acid sequence of SEQ ID NO: 157 and a light chain variable domain having at least 90% identity to the amino acid of SEQ ID NO: 200.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of LVQLVQSGAEVKKPGSSVKVSCKASGGTFNSYAISWVRQAPGQGLEWMGGIIPIFA TANYAQKFQGRVTITADDSTSTAYMELSSLRSEDTAVYYCARVGAPIERTSSSWHYY YYGMDVWGQGTTVTVSS (SEQ ID NO: 158) and comprises a light chain variable region sequence of

(SEQ ID NO: 201)
DIVMTQSPVSLPVTLGEPASISCRSSQSLLHSNGYNYLDWYLQKPGQSP
QLLIYLGCNRASGVPDRFSGSGSGTDFTLKISRVEAEDVGVYYCMQALQ
TPLTLGGGTRVEVK.

In another embodiment, the antibody or antigen binding fragment thereof comprises a heavy chain variable domain having at least 90% identity to the amino acid sequence of SEQ ID NO: 158 and a light chain variable domain having at least 90% identity to the amino acid of SEQ ID NO: 201.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of EVQLVESGGGLIQPGGSLRLSCAASGFTVSSNYMSWVRQAPGKGLEWVSVIYSGG STYYADSVKGRFTISRDNSKNTLYLQMNSLRAEDTAVYYCARDQGEDVFDIWGQGT MVTVSS (SEQ ID NO: 159) and comprises a light chain variable region sequence of

(SEQ ID NO: 202)
DIQLTQSPSFLSASVGDRVTITCRASQGISSYLAWYQQKPGKAPKLIIY
AASTLQSGVPSRFSGSGSGTDFTLTISSLQPEDFATYYCQQLNSYPPGL
TFGGGTKVEIK.

In another embodiment, the antibody or antigen binding fragment thereof comprises a heavy chain variable domain having at least 90% identity to the amino acid sequence of SEQ ID NO: 159 and a light chain variable domain having at least 90% identity to the amino acid of SEQ ID NO: 202.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of QVQLVQSGAEVKKPGSSVKVSCKASGDTFSNYGINWVRQAPGHGLEWMGGIIPLF ATANYAQNFQGRVTITADESTSTAYMELSSLRSEDTAVYYCASFGGDDGLDKWGQ GTLVIVSS (SEQ ID NO: 160) and comprises a light chain variable region sequence of

(SEQ ID NO: 203)
EIVLTQSPASLSLSPGERATLSCRASQSISSYLAWYQQKPGQVPRLLIY
DASSRATGIPARFSGSGSGTDFTLTISSLEPEDFAVYYCQQRSNWPPFT
FGPGTKVDIK.

In another embodiment, the antibody or antigen binding fragment thereof comprises a heavy chain variable domain having at least 90% identity to the amino acid sequence of SEQ ID NO: 160 and a light chain variable domain having at least 90% identity to the amino acid of SEQ ID NO: 203.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of QVQLVQSGAEVKKPGASVKVSCKVSGYTLIELSMHWVRQAPGKGLEWMGGFDPE DGETIYAQKFQGRVTMTEDTSTDTAYMELSSLRSEDTAVYYCTTQGSGPFGEPRG WFDPWGQGTLVTVSS (SEQ ID NO: 161) and comprises a light chain variable region sequence of

(SEQ ID NO: 204)
QSALTQPPSATGSPGQSVTISCTGTSSDVGGYNHVSWYQQHPGKAPKVM
IYEVSKRPSGVPDRFSGSKSGNTASLTVSGLQAEDEADYYCSSYAGRNN
FVVFGGGTKLTVL.

In another embodiment, the antibody or antigen binding fragment thereof comprises a heavy chain variable domain having at least 90% identity to the amino acid sequence of SEQ ID NO: 161 and a light chain variable domain having at least 90% identity to the amino acid of SEQ ID NO: 204.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of EVQLVESGGGLMQPGGSLRLSCAASGITVSRNYMSWVRQAPGKGLEWVSVMYSG GSTYYADSVKGRFTISRDNSKNTLYLQMNSLRAEDTAVYYCARDLDYYGMDVWGQ GTTVTVSS (SEQ ID NO: 162) and comprises a light chain variable region sequence of

(SEQ ID NO: 205)
DIQLTQSPVSLSASVGDRVTITCRASQGISSYLAWYQQKPGKAHKLIIY
AASTLQSGVPSRFSGSGSGTEFTLTISSLQPEDFATYYCQQLNSYLYTF
GQGTKLEIK.

In another embodiment, the antibody or antigen binding fragment thereof comprises a heavy chain variable domain having at least 90% identity to the amino acid sequence of SEQ ID NO: 162 and a light chain variable domain having at least 90% identity to the amino acid of SEQ ID NO: 205.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of QVQLVQSGAEVKKPGASVKVSCKVSGYTLTELSLHWVRQAPGKGLEWMGGFDPE DAETIYAQKFQGRVTMTEDTSTDTAYMELSSLTSEDTAVYYCATLAYCTNGVCYILG PPPLRKHEELYYFYMDVWGKGTTVTVSS (SEQ ID NO: 163) and comprises a light chain variable region sequence of

(SEQ ID NO: 206)
DIQLTQSPSSLSASVGDRVTITCRASQGISSYLAWYQQKPGKAPKLLIY
AASTLQSGVPSRFSGSGSGTEFTLTISNLQPEDFATYYCQQLNSYPRFT
FGPGTKVDIK.

In another embodiment, the antibody or antigen binding fragment thereof comprises a heavy chain variable domain having at least 90% identity to the amino acid sequence of SEQ ID NO: 163 and a light chain variable domain having at least 90% identity to the amino acid of SEQ ID NO: 206.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of LVQLVQSGAEVKKPGASVKVSCKVSGYTLTELSMHWVRQAPGKGLEWMGGFDPE DVETIYAQKFQGRVTMTEDTSTDTAYMELSSLRSEDTAVYFCAASFAVSGTPGPPS HYNYYYGMDVWGQGTTVTVSS (SEQ ID NO: 164) and comprises a light chain variable region sequence of

(SEQ ID NO: 207)
DIVMTQTPLSSPVTLGQPASISCRSSQSLVHSDGNTYLSWLQQRPGQPP
RLLIYKISNRFSGVPDRFSGSGAGTDFTLKISRVEAEDVAVYYCMQATQ
FPYTFGQGTKLEIK.

In another embodiment, the antibody or antigen binding fragment thereof comprises a heavy chain variable domain having at least 90% identity to the amino acid sequence of SEQ ID NO: 164 and a light chain variable domain having at least 90% identity to the amino acid of SEQ ID NO: 207.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of QVQLVQSGAEVKKPGSSAKVSCKASGGSFSRYAVSWVRQAPGQGLEWMGGIIPIF GTPNYAQKFQGRVTITADESTSTAYMELSSLRSEDTAVYYCARTYSTYPKNRLDWD YYYYYGMDVWGQGTTVTVSS (SEQ ID NO: 165) and comprises a light chain variable region sequence of

(SEQ ID NO: 208)
QSALTQPPSASGTPGQRVTISCSGSSSNIGSNFVYWYQQLPGTAPKLLI
YRNHLRPSGIPDRFSGSKSGTSASLAISGLRSEDEADYYCAAWDDSLSG
WVFGGGTKLTVL.

In another embodiment, the antibody or antigen binding fragment thereof comprises a heavy chain variable domain having at least 90% identity to the amino acid sequence of SEQ ID NO: 165 and a light chain variable domain having at least 90% identity to the amino acid of SEQ ID NO: 208.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of QVQLVQSGAEVKKPGASVKVSCKVSGYTLTELSMHWVRQAPGKGLEWMGGFDPE DAETIYAQKFQGRVTMTEDTSTDTAYMELSSLRSEDTAVYYCATGWSITGTPSNYKY YYGMDVWGQGTTVTVSS (SEQ ID NO: 166) and comprises a light chain variable region sequence of

(SEQ ID NO: 209)
DIQMTQSPFSLSAYVGDRVTITCRASQGIRNDLGWYQQKPGKAPKRLIY
VASSFQSGVPSRFSGSGSGTEFTLTISSLQPEDFATYYCLQHNSYPWTF
GQGTKVEIK.

In another embodiment, the antibody or antigen binding fragment thereof comprises a heavy chain variable domain having at least 90% identity to the amino acid sequence of SEQ ID NO: 166 and a light chain variable domain having at least 90% identity to the amino acid of SEQ ID NO: 209.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of DVQLVETGGGLIQPGGSLRLSCAASGFTVSSNYMSWVRQAPGKGLEWVSVIYSGG STYYADSVKGRFTISRDNSKNTLYLQMNSLRAEDTAVYYCARDYGDFYFDYWGQG TLVTVSS SEQ ID NO: 167 and comprises a light chain variable region sequence of

(SEQ ID NO: 210)
EIVLTQSPGTLSLSPGERATLSCRASQSVSSSYLAWYQQKPGQAPRLLI
YGVSSRATGIPDRFSGSGSGTDFTLTISRLEPEDFAVYYCQQYGSSPRT
FGQGTKVEIK.

In another embodiment, the antibody or antigen binding fragment thereof comprises a heavy chain variable domain having at least 90% identity to the amino acid sequence of SEQ ID NO: 167 and a light chain variable domain having at least 90% identity to the amino acid of SEQ ID NO: 210.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of EVQLVESGGGVVQPGRSLRLSCAASGFTFSNYAMHWVRQAPGKGLEWVAVISYDG SNKFYADSVKGRFTISRDNSKNTLYLQINSLRAEDTAVYYCAREELRALADYWGQGT LVTVSS (SEQ ID NO: 168) and comprises a light chain variable region sequence of

(SEQ ID NO: 211)
DIQMTQSPSSLSASVGDRVTITCRASQSLSNYLNWYQHKPGKAPKLLIY
AASSLQSGVPSRFSGSGSGTDFTLTISSLQPEDFATYYCQQSFSPIFTF
GPGTKVDIK.

In another embodiment, the antibody or antigen binding fragment there comprises a heavy chain variable domain having at least 90% identity to the amino acid sequence of SEQ ID NO: 168 and a light chain variable domain having at least 90% identity to the amino acid of SEQ ID NO: 211.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of EVQLVESGGGLVKPGGSLRLSCVGSEFTFSSYSMNWVRQAPGKGLEWVSSISTSS SDIYYADAVKGRFTISRDNAKNSLYLEMNSLRVEDTAVYYCASILEWFAGYMDVWG KGTTVTVSS (SEQ ID NO: 169) and comprises a light chain variable region sequence of

(SEQ ID NO: 212)
SYELTQPPSVSVAPGQTARITCGGNNIGSKSVHWYQQKPGQAPVLVVYD
DSDRPSGIPERFSGSNSGNTATLTISRVEAGDEADYYCQVWDSRSDHPG
GVFGGGTKLTVL.

In another embodiment, the antibody or antigen binding fragment thereof comprises a heavy chain variable domain having at least 90% identity to the amino acid sequence of SEQ ID NO: 169 and a light chain variable domain having at least 90% identity to the amino acid of SEQ ID NO: 212.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of EVQLVESGGGLIQPGGSLRLSCAASGFTVSSNYMSWVRQAPGKGLEWVSVIYSDG RTHYADSVKGRFTISRDNSKNTLFLQMNRLRAEDTAVFYCASPGIVPAAMGVFDYW GQGTLVTVSS (SEQ ID NO: 170) and comprises a light chain variable region sequence of

(SEQ ID NO: 213)
EIVMTQSPATLSVSPGERATLSCRASQSVRSNLAWYQQKPGQAPRLLIY
GASTRATGIPARFSGSGSGTEFTLTISSLQSEDFAVYYCQQYNNWPLTF
GGGTKVEIK.

In another embodiment, the antibody or antigen binding fragment thereof comprises a heavy chain variable domain having at least 90% identity to the amino acid sequence of SEQ ID NO: 170 and a light chain variable domain having at least 90% identity to the amino acid of SEQ ID NO: 213.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of EVQLVESGGGLIQPGGSLRLSCAASGFTVSSNYMSWVRQAPGKGLEWVSLIYSGG STDYADSVKGRFTISRDNSKNTLYLQMNSLRAEDTAVYYCARDLVARGMDVWGQG TTVTVSS (SEQ ID NO: 171) and comprises a light chain variable region sequence of

(SEQ ID NO: 214)
DIQLTQSPSSLSASVGDRVTITCRASQGISSYLAWYQQKPGKAPKLLIY
AASTLQSGVPSRFSGSGSGTEFTLTISSLQPEDFATYYCQQLNSYPQCT
FGPGTKVDIK.

In another embodiment, the antibody or antigen binding fragment thereof comprises a heavy chain variable domain having at least 90% identity to the amino acid sequence of SEQ ID NO: 171 and a light chain variable domain having at least 90% identity to the amino acid of SEQ ID NO: 214.

In another embodiment, the present invention provides an antibody or antigen binding fragment thereof that neutralises SARS-CoV-2 with an IC50 of less than 10ug/ml, wherein the antibody or antigen binding fragment thereof comprises a heavy chain variable region sequence of QVQLQESGPGLVKPSQTLSLTCTVSGGSISSGGYYWSWIRQHPGKGLEWIGYIYYS GSTYYNPSLKSRVIISVDTSKNQFSLKLSSVTAADTAVYFCARDYGGNSNYFGYWG QGTLVTVSS (SEQ ID NO: 172) and comprises a light chain variable region sequence of

(SEQ ID NO: 215)
DIQMTQSPSSLSASVGDRVTITCQASQDISNYLTWYQQKPGKAPKLLIY
DASNLETGVPSRFSGSGSGTDFTFTISSLQPEDIATYYCQQYDNLPLTF
GGGTKVEIK.

In another embodiment, the antibody or antigen binding fragment thereof comprises a heavy chain variable domain having at least 90% identity to the amino acid sequence of SEQ ID NO: 172 and a light chain variable domain having at least 90% identity to the amino acid of SEQ ID NO: 215.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of QVQLLQSGAEVKKPGASVKVSCKVSGYTLSELSMHWVRQAPGKGLEWMGGFDPE DGETIYAQKFQGRVTMTEDTSTDTAYMELNSLRSEDTAVYYCATGFAVAGTSEAYY YHYGMDVWGQGTTVTVSS (SEQ ID NO: 173) and comprises a light chain variable region sequence of

(SEQ ID NO: 216)
QTVVTQEPSLTVSPGGTVTLTCASSTGAVTSGYYPNWFQQKPGQAPRAL
IYSTSNKYSWTPARFSGSLLGGKAALTLSGVQPEDEAEYYCLLYYGGAR
VFGTGTKVTVL.

In another embodiment, the antibody or antigen binding fragment thereof comprises a heavy chain variable domain having at least 90% identity to the amino acid sequence of SEQ ID NO: 173 and a light chain variable domain having at least 90% identity to the amino acid of SEQ ID NO: 216.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of LVQLVQSGAEVKKPGSSVKVSCKASGGTFSSYAISWVRQAPGQGLEWMGGIIPIFG TANYAQKFQGRVTITADESTSTAYMELSSLRSEDTAVYYCARGYSYDNNGWAHWG QGTLVTVSS (SEQ ID NO: 174) and comprises a light chain variable region sequence of

(SEQ ID NO: 217)
DIQMTQSPSSLSASVGDRVTITCQASQDISNYLNWYQQKPGKAPKLLIY
DASNLETGVPSRFSGSGSGTDFTFTISSLQPEDLATYYCQEYDNLPPRR
TFGQGTKVEIK.

In another embodiment, the antibody or antigen binding fragment thereof comprises a heavy chain variable domain having at least 90% identity to the amino acid sequence of SEQ ID NO: 174 and a light chain variable domain having at least 90% identity to the amino acid of SEQ ID NO: 217.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of EVQLVQSGAEVKKPGESLRISCKGSGYSFTSYWISWVRQMPGKGLEWMGRIDPSD SYTNYSPSFQGLVTISADKSISTAYLQWSSLKASDTAMYYCARGGIRSIAVAYEMTAP LYRGFDPWGQGTLVTVSS (SEQ ID NO: 175) and comprises a light chain variable region sequence of

(SEQ ID NO: 218)
DIVMTQSPVSLPVNIGEPASISCRSSQSLLHSNGYNYLDWYLQKPGQSP
QLLISLGCNRASGVPDRFSGSGSGTDFTLKISRVEAEDVGVYSCMQALQ
TPLTFGGGTKVEIK.

In another embodiment, the antibody or antigen binding fragment thereof comprises a heavy chain variable domain having at least 90% identity to the amino acid sequence of SEQ ID NO: 175 and a light chain variable domain having at least 90% identity to the amino acid of SEQ ID NO: 218.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of EVQLVESGGGVVQPGRSLRVSCAASGFDFSGFGMHWVRQAPGKGLEWVAIIWYD GSNKNYADSVKGRFTISRDNSKNTLYLQMNSLRAEDTAVYYCAREWHHGSGEGYM DVWGKGTTVTVSS (SEQ ID NO: 176) and comprises a light chain variable region sequence of

(SEQ ID NO: 219)
DIVMTQTPLSLPVTPGEPASISCRSSQSLLDSDDGNTYLDWYLQKPGQS
PQLLIYTLSYRASGVPDRFSGSGSGTDFTLKISRVEAEDVGVYYCMQRI
EFPLTFGPGTKVDIK.

In another embodiment, the antibody or antigen binding fragment thereof comprises a heavy chain variable domain having at least 90% identity to the amino acid sequence of SEQ ID NO: 176 and a light chain variable domain having at least 90% identity to the amino acid of SEQ ID NO: 219.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of EVQLLESGGGLVQPGGSLRLSCAASGFTFSSYAMSWVRQAPGKGLEWVSAISGSG GSTYYADSVKGRFTISRDNSKITLYLQVNSLRAEDTAVYYCAKGVVPEAIPWFDPWG QGILVTVSS (SEQ ID NO: 177) and comprises a light chain variable region sequence of

(SEQ ID NO: 220)
EIVLTQSPAPLSLSPGERATLSCRASQSVSSYLAWYQQKPGQAPRLLIY
DASNRATGIPARFSGSGSGTDFTLTISSLEPEDFAVYYCQQRSNWPPTF
GQGTKVEIK.

In another embodiment, the antibody or antigen binding fragment thereof comprises a heavy chain variable domain having at least 90% identity to the amino acid sequence of SEQ ID NO: 177 and a light chain variable domain having at least 90% identity to the amino acid of SEQ ID NO: 220.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of QVQLQESGPGLVKPSQTLSLTCTVSGGSISSGGYYWSWIRQHPGKGLEWIGYIYYS GSTYYNPSLKSRVTISVDTSKNQFSLKLSSVTAADTAVYYCARVVGSSWPWGFDYW GQGTLVTVSS (SEQ ID NO: 178) and comprises a light chain variable region sequence of

(SEQ ID NO: 221)
EIVMTQSPATLSVSPGERATLSCRASQSISSNLAWYQQKPGQAPRLLIY
GASTRATGVPARFSGSGSGTDFTLTISSLQSEDFAVYYCQQYNNWWTFG
QGTKVEIK.

In another embodiment, the antibody or antigen binding fragment thereof comprises a heavy chain variable domain having at least 90% identity to the amino acid sequence of SEQ ID NO: 178 and a light chain variable domain having at least 90% identity to the amino acid of SEQ ID NO: 221.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of QVQLQESGPGLVKPSGTLSLTCAVSNGSVSSINWWSWVRQPPGKGLEWIGEIYHS GSTNYNPSLKSRVTISVDKSKNQISLEMNSVTAADTAFYYCARDRSGGPHGGFDPW GQGTLVTVSS (SEQ ID NO: 179) and comprises a light chain variable region sequence of

(SEQ ID NO: 222)
QSALTQPASVSGSPGQSITISCTGTSSDVGGYNYVSWYQQHPGKAPKLM
IYDVSNRPSGVSNRFSGSKSGNTASLTISGLQAEDEADYYCSSYTSSNT
LAVVFGGGTKLTVL.

In another embodiment, the antibody or antigen binding fragment thereof comprises a heavy chain variable domain having at least 90% identity to the amino acid sequence of SEQ ID NO: 179 and a light chain variable domain having at least 90% identity to the amino acid of SEQ ID NO: 222.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of EVQLVESGGGLVKPGGSLRLSCAASGFSFSDADMSWVRQAPGKGLEWVGRIKTRI AGGTVDYAPPVKGRFTISRDDSKNTLYLQMNSLKTEDTGVYYCTTEGVQSSGWYLS FDFWGQGTLVTVSS (SEQ ID NO: 180) and comprises a light chain variable region sequence of

(SEQ ID NO: 223)
DIQMTQSPFSLSASVGDRVTITCRASQTISNYLNWYQQKPGKAPKLLMY
AASTLQSGVPSRFSGSGSGTDFTLTISSLQPEDFATYYCQQSYSTLYTF
GQGTKLEIK.

In another embodiment, the antibody or antigen binding fragment thereof comprises a heavy chain variable domain having at least 90% identity to the amino acid sequence of SEQ ID NO: 180 and a light chain variable domain having at least 90% identity to the amino acid of SEQ ID NO: 223.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of LVQLVQSGAEVKKPGASVKVSCKASGYTFTRYAMHWVRQAPGQRLEWMGWINAG NGNTKYSEKFQGRVTITRDTSASTAYMELSSLRSEDTAVYHCARGLYYYDRIGSQST EDYFDYWGQGTLVTVSS (SEQ ID NO: 181) and comprises a light chain variable region sequence of

(SEQ ID NO: 224)
QSALTQPPSVSGAPGQRVTISCTGSSSNIGAGYDVHWYQQLPGTAPKLL
IYGNSNRPSGVPDRFSGSKSGTSASLAITGLQAEDEADYYCQSYDSSLS
APYVFGTGTKVTVL.

In another embodiment, the antibody or antigen binding fragment thereof comprises a heavy chain variable domain having at least 90% identity to the amino acid sequence of SEQ ID NO: 181 and a light chain variable domain having at least 90% identity to the amino acid of SEQ ID NO: 224.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of QVQLVQSGAEVKKPGASVKVSCKVSGYTLTELSMHWVRQAPGKGLEWMGGFDPE DAETIYAQKFQGRVTMTEDTSTDTAYMELSSLRSEDTAVYYCATSTAVRGERPGGY NYYYGMDVWGQGTTVTVSS (SEQ ID NO: 182) and comprises a light chain variable region sequence of

(SEQ ID NO: 225)
DIVMTQTPLSSPVTLGQPASISCRSSQSFVHSDGNTYLSWLQQRPGQPP
RLLIYKISGRFCGVPDRFSGSGAGTDFTLKISRVEAEDVGVYYCMQVTQ
FPHTFGQGTKLEIK.

In another embodiment, the antibody or antigen binding fragment thereof comprises a heavy chain variable domain having at least 90% identity to the amino acid sequence of SEQ ID NO: 182 and a light chain variable domain having at least 90% identity to the amino acid of SEQ ID NO: 225.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of EVQLVQSGAEVKKPGESLRISCKGSGYSFTSYWISWVRQMPGKGLEWMGRIDPSD SYTNYSPSFQGHVSISADKSISTAYLQWSSLKASDTAMYYCARHELVCSGGSCYLE EYFQYWGQGTLVTVSS (SEQ ID NO: 183) and comprises a light chain variable region sequence of

(SEQ ID NO: 226)
QCVLTRPPSVSAAPGQKVTISCSGSSSNIGNNYVSWYQQLPGTAPKLLI
YDNNKRPSGIPDRFSGSKSGTSATLGITGLQTGDEADYYCGTWDSSLSA
GVFGGGTKLTVL.

In another embodiment, the antibody or antigen binding fragment thereof comprises a heavy chain variable domain having at least 90% identity to the amino acid sequence of SEQ ID NO: 183 and a light chain variable domain having at least 90% identity to the amino acid of SEQ ID NO: 226.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of QVQLVQSGAEVKKPGASVKVSCKASGYTFTGYYMNWVRQAPGQGLEWMGWINPN SGGTKYIQKFQGRVTMTRDTSISTAYMELSRLRSDDTALYYCARSPVADPWELLRAT YYYYGMDVWGQGTTVTVSS (SEQ ID NO: 184) and comprises a light chain variable region sequence of

(SEQ ID NO: 227)
DIQMTQSPSSVYASVGDRVILTCRASQGISRWLAWYQQKPGKARKLLLY
AASSLQSGVPSRFSGSGSGTDFTLTINSLQPEDFATYYCLQANSFPYTF
GQGTKLEIK.

In another embodiment, the antibody or antigen binding fragment thereof comprises a heavy chain variable domain having at least 90% identity to the amino acid sequence of SEQ ID NO: 184 and a light chain variable domain having at least 90% identity to the amino acid of SEQ ID NO: 227.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of VVQLVQSGAEVKKPGESVRISCKGSGYSFTTYWISWVRQMPGKGLEWMGRIDPSD SYTHYSPSFEGHVTISVDKSVSTAYLQWSSLKASDTAMYYCARQGRWLVPEAEGA WDYNYMDVWGEGTTVTVSS (SEQ ID NO: 185) and comprises a light chain variable region sequence of

(SEQ ID NO: 228)
DIQMTQSPSSLSASVGDRVTITCRASQSIGSWLAWYQQKPGKAPKLLIY
KASSLESGVPSRFSGSGSGTEFTLTISSLQPDDFATYYCQQYNSYFMFS
FGQGTKLEIK.

In another embodiment, the antibody or antigen binding fragment thereof comprises a heavy chain variable domain having at least 90% identity to the amino acid sequence of SEQ ID NO: 185 and a light chain variable domain having at least 90% identity to the amino acid of SEQ ID NO: 228.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of EVQLVESGGGVVQPGRSLRLSCAASGFTFSTYGMHWVRQAPGKGLEWVAVILYDG SDKYYADSVKGRFTISRDNSKNTLFLQMNSLRAEDTAVYYCVKGGWYGDSLRVDY WGQGTLVTVSS (SEQ ID NO: 186) and comprises a light chain variable region sequence of

(SEQ ID NO: 229)
DIQMTQFPFSLFASVGDRVTITCQASQDISNYLNWYQQKPGKAPKLLIY
DASNLETGVPSRFSGSGSGTDFTFTISSLQPEDIATYYCQQYDTLPPIT
FGQGTRLEIK.

In another embodiment, the antibody or antigen binding fragment thereof comprises a heavy chain variable domain having at least 90% identity to the amino acid sequence of SEQ ID NO: 186 and a light chain variable domain having at least 90% identity to the amino acid of SEQ ID NO: 229.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of QVQLQESGPGLVKPSETLSLTCSVSGGSISSNSYYWGWIRQPPGKGLEWIGSMYYS GSTSYNPSLKSRVTISVDTSKNQFSLKLSSVTAADTAVYYCASASKRGYSGYDFISP YYFDYWGQGTLVTVSS (SEQ ID NO: 187) and comprises a light chain variable region sequence of

(SEQ ID NO: 230)
QSVLTQPPWAYGTPGERFTISCSGSSSNIGSNTVNWYQQLPGTAPKLLI
YSNNQRPSGVVDRFSGSKCGTSASLAISGLQSDDEADYYCAAWDDSMIG
PVFGGGTKLT.

In another embodiment, the antibody or antigen binding fragment thereof comprises a heavy chain variable domain having at least 90% identity to the amino acid sequence of SEQ ID NO: 187 and a light chain variable domain having at least 90% identity to the amino acid of SEQ ID NO: 230.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of EVQLVESGGGLVKPGGSLRLSCAASGFSFSNAWMTWVRQAPGKGLEWVGRIKTKI DGGATEYAAPVKGRFTISRDDTKNTLYLQMSSLKTEDTAVYYCTTTVSGWFWGDAF DIWGQGTMVTVSS (SEQ ID NO: 188) and comprises a light chain variable region sequence of

(SEQ ID NO: 231)
DIQMTQSPSSLSASVGDRVTITCRASQTIGNYVIWYQQKPGKAPKLLIY
AASSLQSGVPSRFSGSGSGTDFTLTISSLQPEDLATYYCQQSYNTPRTF
GQGTKVEIK.

In another embodiment, the antibody or antigen binding fragment thereof comprises a heavy chain variable domain having at least 90% identity to the amino acid sequence of SEQ ID NO: 188 and a light chain variable domain having at least 90% identity to the amino acid of SEQ ID NO: 231.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of EVQLVESGGGLVKPGGSLRLSCAASEFTFSNYTMNWVRQAPGKGLEWVSSISSSS SFIYYADSVKGRFTISRDNAKNSLYLQMNSLRAEDTAVYYCARDPSLHRFFRYYYIDV WGKGTTVTVSS (SEQ ID NO: 189) and comprises a light chain variable region sequence of

(SEQ ID NO: 232)
QSALTQPASVSGSPGQSITISCTGTSSDVGSYNLVSWYQQHPGKAPKLM
IYEVSKRPSGVSDRFSGSKSGNTASLTISGLQAEDEADYYCCSYAGSST
LKVFGGGTKLTVL.

In another embodiment, the antibody or antigen binding fragment thereof comprises a heavy chain variable domain having at least 90% identity to the amino acid sequence of SEQ ID NO: 189 and a light chain variable domain having at least 90% identity to the amino acid of SEQ ID NO: 232.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of QVQLQESGPGLVKPSETLSLTCTLYGASISSYYWSWIRQPPGKGLEWIGYMYYSGS TNYNPSLKSRVTISVDTSKNQFSLNLSSVTAADTAVYYCARDREGGYCSGGRCYSS HYMDVWGKGTTVTVSS (SEQ ID NO: 190) and comprises a light chain variable region sequence of

(SEQ ID NO: 233)
SYELTQPPSVSVAPGKTARITCGENNIGNKSVNWYQQKPGQAPVLVIYY
DSDRPSGIPERFSGSNSGNTATLTISWVEAGDEADYYCQVWDSSSDHVV
FGGGTKLTDI.

In another embodiment, the antibody or antigen binding fragment thereof comprises a heavy chain variable domain having at least 90% identity to the amino acid sequence of SEQ ID NO: 190 and a light chain variable domain having at least 90% identity to the amino acid of SEQ ID NO: 233.

In another embodiment, the present invention provides an antibody or antigen binding fragment thereof that neutralises SARS-CoV-2 with an IC50 of less than 50ug/ml, wherein the antibody or antigen binding fragment thereof comprises a heavy chain variable region sequence of QVQLVQSGAEVKKPGASMKVSCKASGYTLANYYIHWVRQAPGQGPEWMGIINPSG GATTYAQKFQGRVTMTSDTSTSTVYMELSSLRSEDTAVYYCARGGATPHLRGGMD VWGKGTTVTVSS (SEQ ID NO: 191) and comprises a light chain variable region sequence of

(SEQ ID NO: 234)
DIQMTQSPFSLSASVGDRVTITCRASQSISTWLAWFQQKPGKAPKLLIY
KASSLESGVPSRFSGSGSGTEFTLTISSLQPDDFATYYCQQYNSYWTFG
QGTKVEIK.

In another embodiment, the antibody or antigen binding fragment thereof comprises a heavy chain variable domain having at least 90% identity to the amino acid sequence of SEQ ID NO: 191 and a light chain variable domain having at least 90% identity to the amino acid of SEQ ID NO: 234.

In another embodiment, the present invention provides an antibody or antigen binding fragment thereof that neutralises SARS-CoV-2 with an IC50 of less than 100ug/ml, wherein the antibody or antigen binding fragment thereof comprises a heavy chain variable region sequence of EVQLVESGGGVVQPGRSLRLSCAASGFTFSSYGMHWVRQAPGKGLEWVAVIWYD GSNKYYADSVKGRFTISRDNSKNTLYLQMNSLRAEDTAVYYCARATMAAIRGGWFD PWGQGTLVTVSS (SEQ ID NO: 192) and comprises a light chain variable region sequence of

(SEQ ID NO: 235)
DIVMTQTPLSLSVTPGQPASISCKFSQSLLHSDGKTYLYWYLQKPGQPP
QLLIYEVSNRFSGVPDRFSGSGSGTDFTVKISRVEAEDVGVYYCMQSIQ
LWTFGQGTKVEIK.

In another embodiment, the antibody or antigen binding fragment thereof comprises a heavy chain variable domain having at least 90% identity to the amino acid sequence of SEQ ID NO: 192 and a light chain variable domain having at least 90% identity to the amino acid of SEQ ID NO: 235.

In another embodiment, the present invention provides an antibody or antigen binding fragment thereof that neutralises SARS-CoV-2 with an IC50 of less than 100ug/ml, wherein the antibody or antigen binding fragment thereof comprises a heavy chain variable region sequence of EVQLVESGGGVVQPGRSLRLSCAASGFTFSNFDMHWVRQAPGKGLEWLAIISSDAT SKFYADSVKGRITISRDNSKNTLNLLVNSLRAEDTAVYYCARDQGGSYDYVGGSYRL GPTYFDYWGQGTPVTVSS (SEQ ID NO: 193) and comprises a light chain variable region sequence of

(SEQ ID NO: 236)
DIQMTQSPSSLSASVGDRVTITCRASQSIGNYLNWYQQKPGKAPKLLIY
AASRLQSGVPSRFSGSGSGTDFTLTISSLQPEDFATYYCQQSYSTLALS
FGGGTKVEIK.

In another embodiment, the antibody or antigen binding fragment thereof comprises a heavy chain variable domain having at least 90% identity to the amino acid sequence of SEQ ID NO: 193 and a light chain variable domain having at least 90% identity to the amino acid of SEQ ID NO: 236.

In another embodiment, the present invention provides an antibody or antigen binding fragment thereof that neutralises SARS-CoV-2 with an IC50 of less than 100ug/ml, wherein the antibody or antigen binding fragment thereof comprises a heavy chain variable region sequence of QVQLQESGPGLVKPSQTLSLTCTVSGGSISSGGYYWSWIRQHPGKGLEWIGYIYSS GSTYYNPSLKSRVTISVDTSKNQFSLKLSSVTAADTAVYYCARVSDFDWFDPWGQG TLVTVSS (SEQ ID NO: 194) and comprises a light chain variable region sequence of

(SEQ ID NO: 237)
EIVLTQSPATLSLSPGERATLSCRASQSVSSYLAWYQQKPGQAPRLLIY
DASNRATGIPARFSGSGSGTDFTLTISSLEPEDFAVYYCQQRFNWPPIT
FGQGTRLEIK.

In another embodiment, the antibody or antigen binding fragment thereof comprises a heavy chain variable domain having at least 90% identity to the amino acid sequence of SEQ ID NO: 194 and a light chain variable domain having at least 90% identity to the amino acid of SEQ ID NO: 237.

In another embodiment, the present invention provides an antibody or antigen binding fragment thereof that neutralises SARS-CoV-2 with an IC50 of less than 100ug/ml, wherein the antibody or antigen binding fragment thereof comprises a heavy chain variable region sequence of EVQLVESGGGVVQPGRSLRLSCAASGFTLSTFGMHWVRQAPGKGLEWVAVISYDG SKKYYADSVKGRFTISRDNSKNTLYLQMNSLRAEDTAVYYCAKDLYYYDTGAPYYF DYWGQGTLVTVSS (SEQ ID NO: 195) and comprises a light chain variable region sequence of

(SEQ ID NO: 238)
EIVLTQSPVSLSLSPGERATLSCRASPSVGTYLAWYQQRPGQAPRLLIY
DASNRATGIPARFSGSGSGTDFTLTISSLEPEDFAVYYCQQRSNWPLIT
FGQGTRLEIK.

In another embodiment, the antibody or antigen binding fragment thereof comprises a heavy chain variable domain having at least 90% identity to the amino acid sequence of SEQ ID NO: 195 and a light chain variable domain having at least 90% identity to the amino acid of SEQ ID NO: 238.

In another embodiment, the present invention provides an antibody or antigen binding fragment thereof that neutralises SARS-CoV-2 with an IC50 of less than 200ug/ml, wherein the antibody or antigen binding fragment thereof comprises a heavy chain variable region sequence of EVQLVESGGGLIQPGGSLRLSCAASGFTVSSNYMSWVRQAPGKGLEWVSVIYSGG STYYADSVKGRFTVSRDNSKNTLYLQMNSLRAEDTAVYYCARGGRYDYDVFDIWG QGTMVTVSS (SEQ ID NO: 196) and comprises a light chain variable region sequence of

(SEQ ID NO: 239)
DIQLTPSSFSLSASVGDRVTITCRASQGISSYLAWYQQKPGKAPKLLIY
AASTLQSGVPSRFSGSGSGTEFTLTISSLQPEDFATYYCQQLNSYPGLT
FGGGTKVEIK.

In another embodiment, the antibody or antigen binding fragment thereof comprises a heavy chain variable domain having at least 90% identity to the amino acid sequence of SEQ ID NO: 196 and a light chain variable domain having at least 90% identity to the amino acid of SEQ ID NO: 239.

In another embodiment, the present invention provides an antibody or antigen binding fragment thereof that neutralises SARS-CoV-2 with an IC50 of less than 200ug/ml, wherein the antibody or antigen binding fragment thereof comprises a heavy chain variable region sequence of EVQLVESGGDLVQPGGSLRLSCAASGFTFSSYDMHWVRQATGKGLEWVSTIGTTG DTYYPGSVKGRFTISRENAKNSLYLQMNSLRAEDTAVYYCARGTHIYHHERERGYY YYYMDVWGKGTTVTVSS (SEQ ID NO: 197) and comprises a light chain variable region sequence of

(SEQ ID NO: 240)
DIQMTQSPSSLSASVGDRVTITCRASQSISSYLNWYQQKPGKAPKLLIY
AASSLQSGVPSRFSGSGSGTDFTLTISNLQPADFATYYCQQSYSTPQYT
FGQGTKLEIK.

In another embodiment, the antibody or antigen binding fragment thereof comprises a heavy chain variable domain having at least 90% identity to the amino acid sequence of SEQ ID NO: 197 and a light chain variable domain having at least 90% identity to the amino acid of SEQ ID NO: 240.

In another embodiment, the present invention provides an antibody or antigen binding fragment thereof that neutralises SARS-CoV-2 with an IC50 of less than 200ug/ml, wherein the antibody or antigen binding fragment thereof comprises a heavy chain variable region sequence of EVQLVESGGGVVQPGRSLRLSCAASGFTFSNYGMHWVRQAPGKGLEWVAVISYD GSNKYSADSVKGRFTISRDNSKNTLYLQMNSLRAEDTAVYYCAKVQDPYCSPTNCY TSNYYFDDWGQGTLVTVSS (SEQ ID NO: 198) and comprises a light chain variable region sequence of

(SEQ ID NO: 241)
DIQLTQSPFSLSASVGDRVTITCRASQGISSYLAWYQQKPGKAPKLLIY
AASTLQSGVPSRFSGSGSGTEFTLTISSLQPEDFATYYCQQLGTTFGQG
TRLEIK.

In another embodiment, the antibody or antigen binding fragment thereof comprises a heavy chain variable domain having at least 90% identity to the amino acid sequence of SEQ ID NO: 198 and a light chain variable domain having at least 90% identity to the amino acid of SEQ ID NO: 241.

In another embodiment, the present invention provides an antibody or antigen binding fragment thereof that neutralises SARS-CoV-2 with an IC50 of less than 200ug/ml, wherein the antibody or antigen binding fragment thereof comprises a heavy chain variable region sequence of EVQLVESGGGVVQPGRSLRLSCAASGFTFTNYGMHWVRQAPGKGLEWVAVISYD GSNKYYADSVKGRFTISRDNSKNTLYLQMNSLRTEDTAVYYCTKCGSPYCSGGSCY SCWFDPWGQGTLVIVSS (SEQ ID NO: 199) and comprises a light chain variable region sequence of

(SEQ ID NO: 242)
DIQMTQSPSSLSASVGDRVTITCQASQDISNYLNWYQQKPGKAPKLLIY
DASNLETGVPSRFSGSGSGTDFTFTISSLQPEDIATYYCQQSDNLPFFG
PGTKVDIK.

In another embodiment, the antibody or antigen binding fragment thereof comprises a heavy chain variable domain having at least 90% identity to the amino acid sequence of SEQ ID NO: 199 and a light chain variable domain having at least 90% identity to the amino acid of SEQ ID NO: 242.

While the exemplified antibodies or fragments thereof are human monoclonal antibodies, in one embodiment the antibody or antigen binding fragment thereof may be any isotype, including IgG, IgM, IgE, IgD, or IgA, and any subtype thereof. Preferably, the antibody or antigen binding fragment thereof of the invention is a human IgG or variant thereof, preferably human IgG4 or variant thereof. Methods to switch the type of antibody are well known in the art. The nucleic acid molecule encoding the VH or VL region is isolated, and operatively linked to a nucleic acid sequence encoding a different CH or CL, respectively, from the constant region of a different class of immunoglobulin molecule.

The present disclosure encompasses proteins and/or antibodies described herein comprising a constant region of an antibody. This includes antigen binding fragments of an antibody fused to a Fc.

Sequences of constant regions useful for producing the proteins of the present disclosure may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4.

In one embodiment, the constant region is human isotype IgG4 or a stabilized IgG4 constant region.

In one embodiment, the Fc region of the constant region has a reduced ability to induce effector function, e.g., compared to a native or wild-type human IgG1 or IgG3 Fc region. In one embodiment, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are well known in the art.

In one embodiment, the Fc region is an IgG4 Fc region (i.e., from an IgG4 constant region), e.g., a human IgG4 Fc region. Sequences of suitable IgG4 Fc regions will be apparent to the skilled person and/or available in publicly available databases (e.g., available from National Center for Biotechnology Information).

In one embodiment, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

The present disclosure also contemplates additional modifications to an antibody. For example, the antibody comprises one or more amino acid substitutions that increase the half-life of the protein. For example, the antibody comprises an Fc region comprising one or more amino acid substitutions that increase the affinity of the Fc region for the neonatal Fc region (FcRn).

Importantly, the present inventors have demonstrated a number of antibodies surprisingly showed better neutralisation or binding than REGN10933 and REGN10987 against numerous SARS-CoV-2 variants (including Omicron variants) and RBDs with substitutions present in spike proteins, including VOC spike proteins). For example, see FIGS. 1, 6, 19, 22 and 23.

In one embodiment, the antibody is not REGN10933 and REGN10987.

The antibody used in the context of the present invention may be produced by any method well-known in the art. For example, the antibody may be produced by introducing a nucleic acid encoding the antibody into a suitable cell, e.g., a mammalian cell line, such as CHO, HEK293, MDCK, COS, HeLa, or myeloma cell lines such as NS0. Another suitable cell line is an insect cell line for use with a baculovirus, such as SF9 cells, SF21 cells, or HighFive™ cells. Yet another cell is a yeast cell, such as Saccharomyces, e.g. S. cerevisiae, or Pichia pistoris. Bacterial host cells such as E. coli are also possible. Methods for introducing DNA into the respective host cells are well known in the art. For example, when the host cell is a mammalian cell line, techniques such as lipofection or electroporation may be used.

The method of producing the antibody may comprise culturing the host cells, such as the cell line or yeast cell, of the invention under appropriate conditions to express the antibody. The antibody may then be purified. The antibody may be secreted by the host cell, and can then easily be purified from the culture supernatant. Techniques for purifying antibodies are well known in the art, and include techniques such as ammonium sulfate precipitation, size exclusion chromatography, affinity chromatography, ion exchange chromatography and others.

When expressed in E. coli, the antibodies or antigen binding fragments thereof may be produced in inclusion bodies. Methods to isolate inclusion bodies and refold the expressed protein are well known in the art.

The present invention also relates to an anti-SARS-CoV-2 antibody for use in a method of treating or preventing COVID-19 in a subject, wherein the antibody is administered to the subject, and wherein the anti-SARS-CoV-2 is obtained by introducing a nucleic acid encoding the anti-SARS-CoV-2 as disclosed above into a cell, preferably the nucleic acids encoding the light and heavy chains described herein, or light and heavy chains comprising the CDRs described herein, and the anti-anti-SARS-CoV-2 antibody is produced in the cell and is subsequently purified.

For example, for the preparation of the pharmaceutical composition for subcutaneous administration, the antibody can be mixed with one or more pharmaceutically acceptable carriers, diluents or excipients. For example, sterile water or physiological saline may be used. Other substances, such as pH buffering solutions, viscosity reducing agents, or stabilizers may also be included.

A wide variety of pharmaceutically acceptable excipients and carriers are known in the art. Such pharmaceutical carriers and excipients as well as suitable pharmaceutical formulations have been amply described in a variety of publications (see for example “Pharmaceutical Formulation Development of Peptides and Proteins”, Frokjaeret al., Taylor & Francis (2000) or “Handbook of Pharmaceutical Excipients”, 3rd edition, Kibbe et al., Pharmaceutical Press (2000) A. Gennaro (2000) “Remington: The Science and Practice of Pharmacy”, 20th edition, Lippincott, Williams, & Wlkins; Pharmaceutical Dosage Forms and Drug Delivery Systems (1999) H. C. Ansel et al., eds 7th ed., Lippincott, Wiliams, & Wilkins; and Handbook of Pharmaceutical Excipients (2000) A. H. Kibbe et al., eds., 3rd ed. Amer. Pharmaceutical Assoc). In particular, the pharmaceutical composition comprising the antibody of the invention may be formulated in lyophilized or stable soluble form. The polypeptide may be lyophilized by a variety of procedures known in the art. Lyophilized formulations are reconstituted prior to use by the addition of one or more pharmaceutically acceptable diluents such as sterile water for injection or sterile physiological saline solution.

Pharmaceutical compositions comprising an antibody or fragments thereof described herein can be administered in dosages and by techniques well known in the art. The amount and timing of the administration will be determined by the treating physician to achieve the desired purposes and should ensure a delivery of a safe and therapeutically effective dose to the blood of the subject to be treated.

In one embodiment, the anti-SARS-CoV-2 antibody or fragment thereof is administered in an amount to maintain an effective concentration of the neutralising antibody in the blood.

The term “therapeutically effective amount” includes an amount of an anti-SARS-CoV-2 antibody or fragment thereof described herein that is effective for preventing, ameliorating and/or treating a condition resulting from infection with SARS-CoV-2.

In one embodiment, the anti-SARS-CoV-2 antibody or fragment thereof is administered in an amount to maintain an effective concentration of the neutralising antibody in the blood.

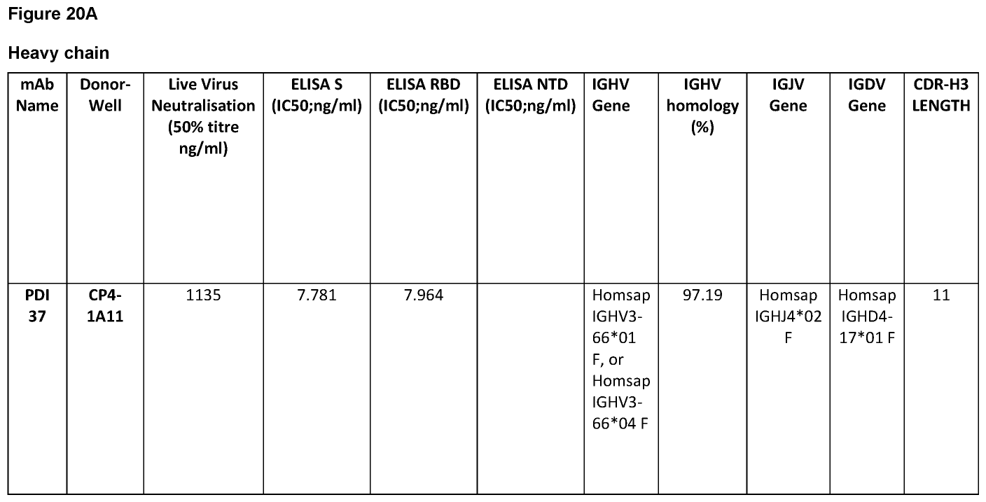

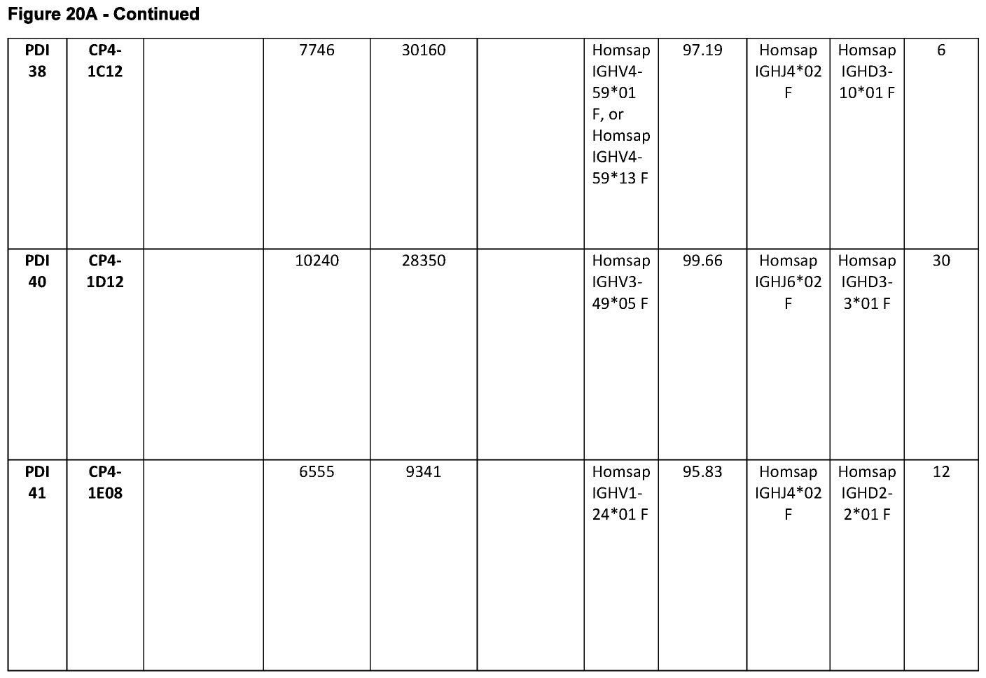

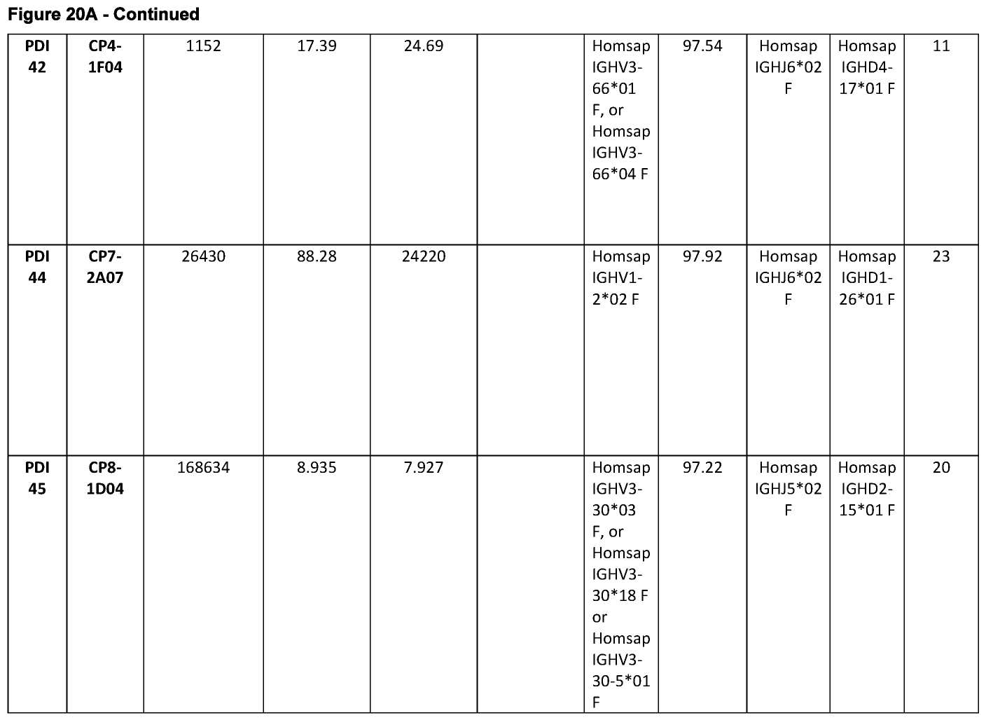

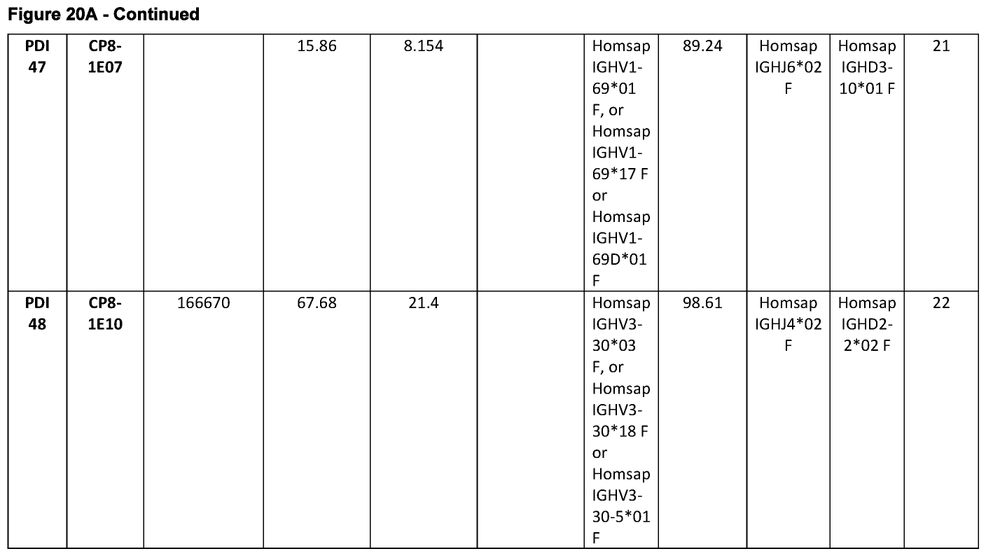

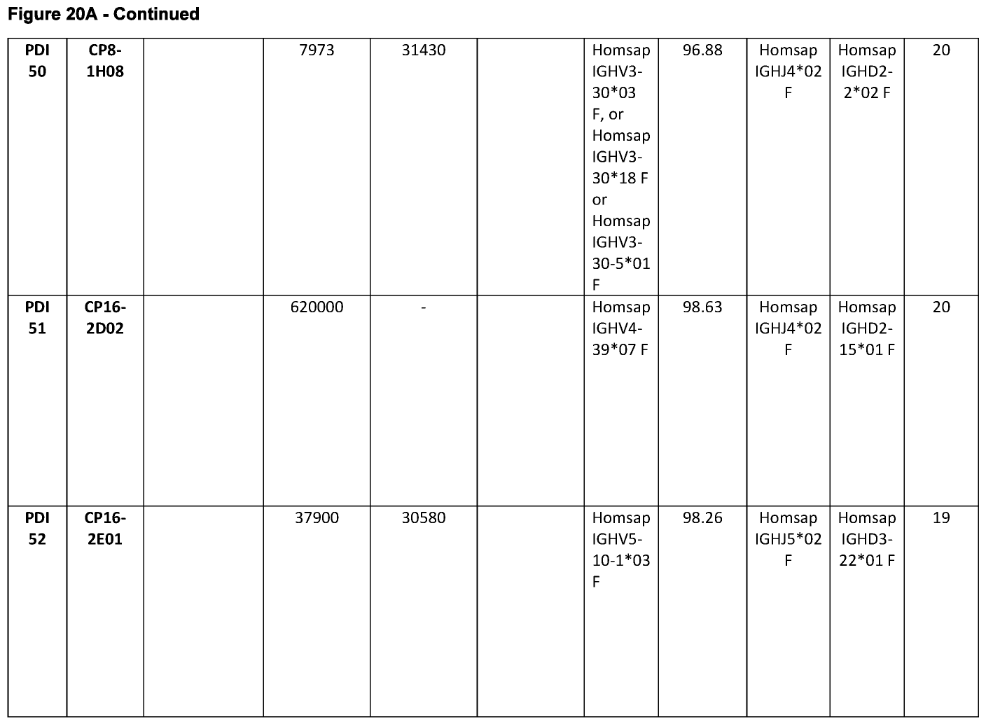

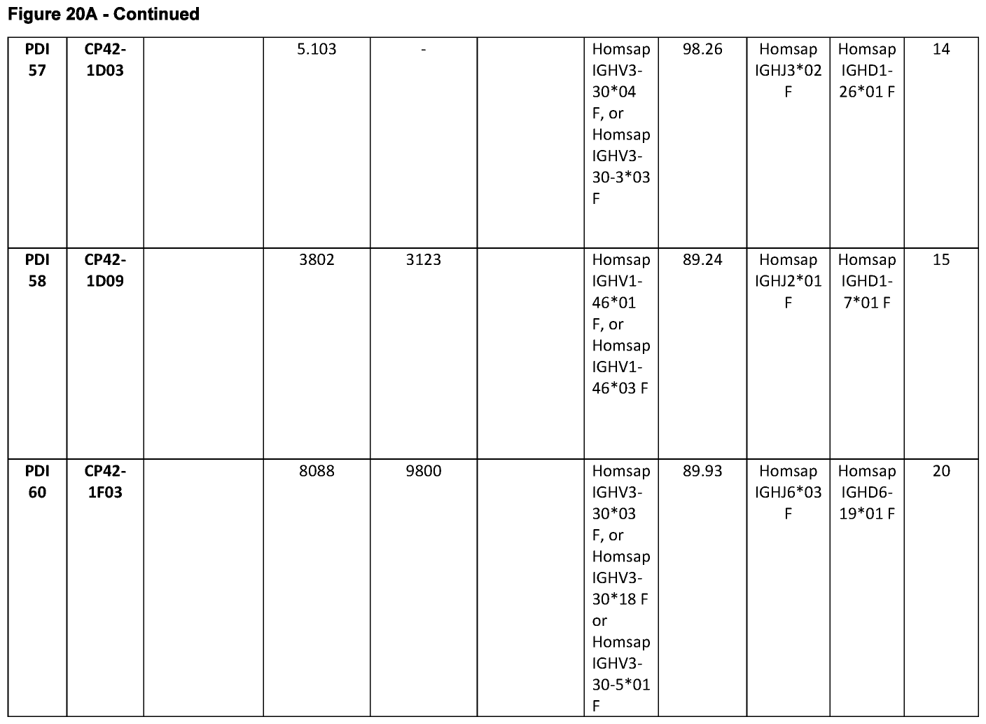

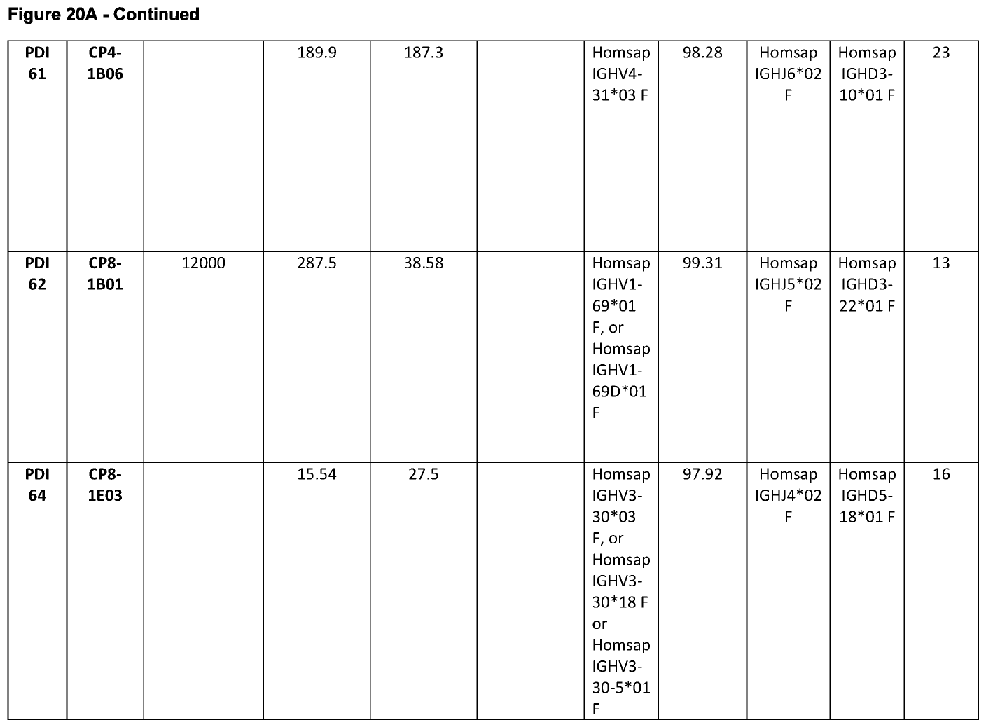

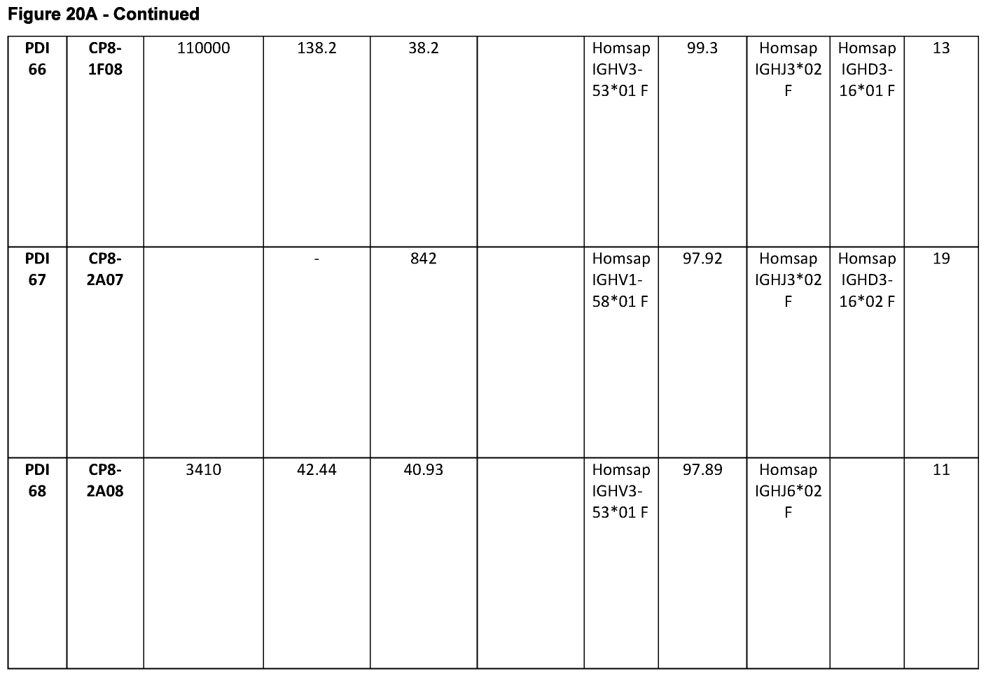

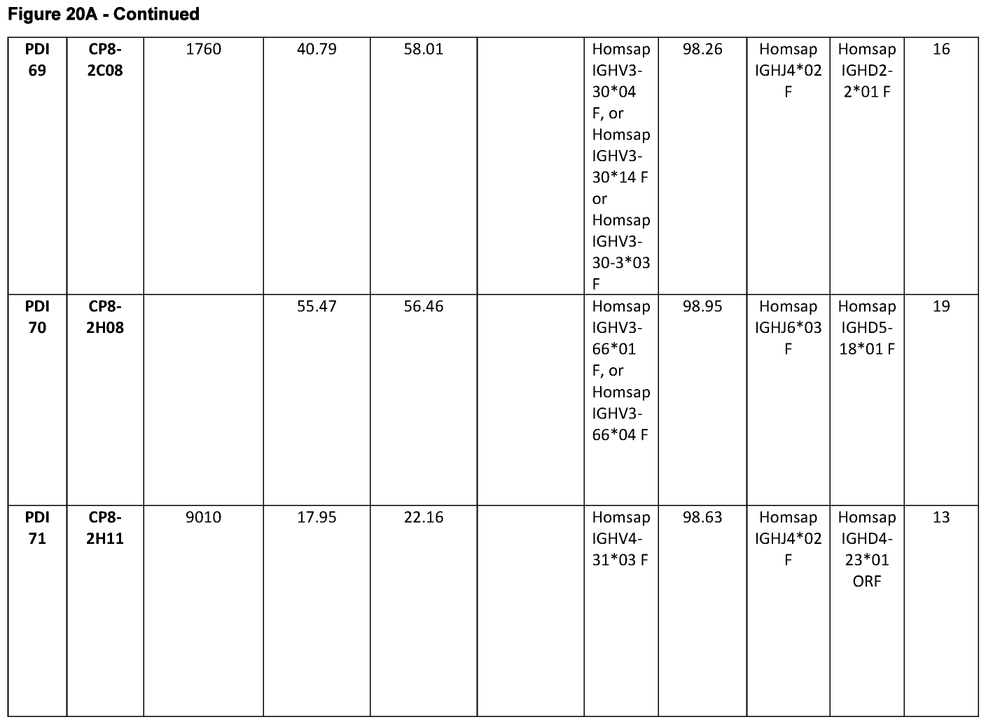

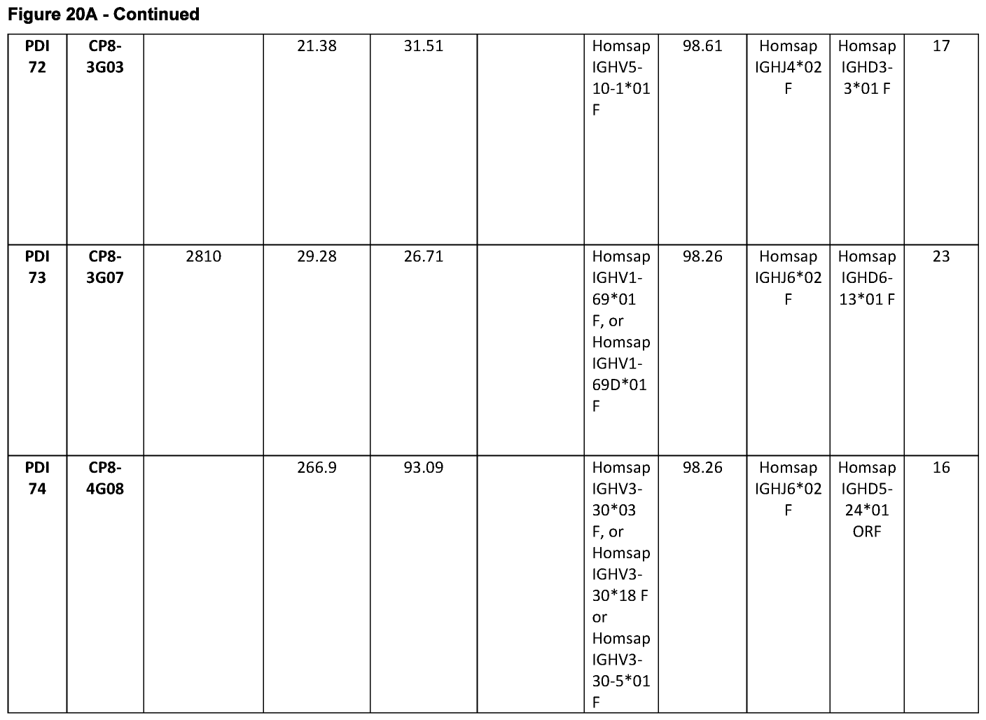

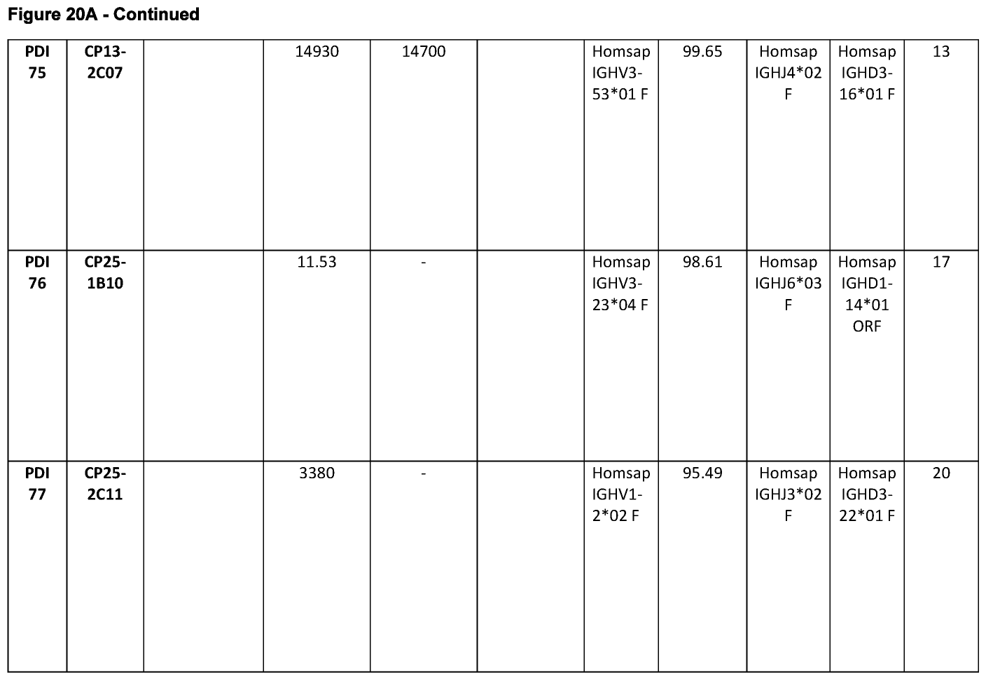

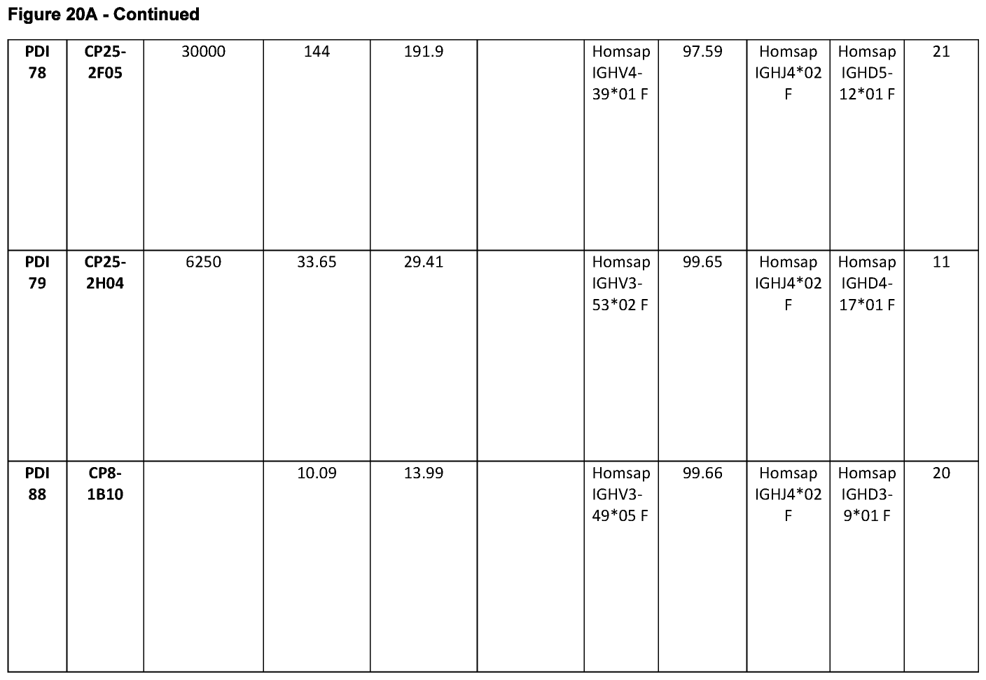

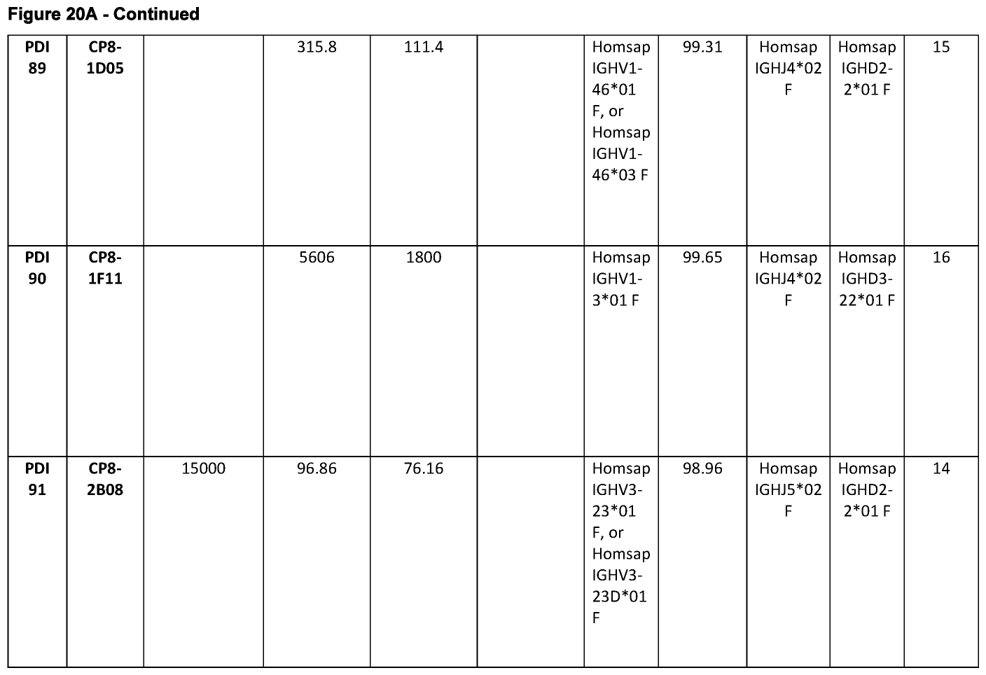

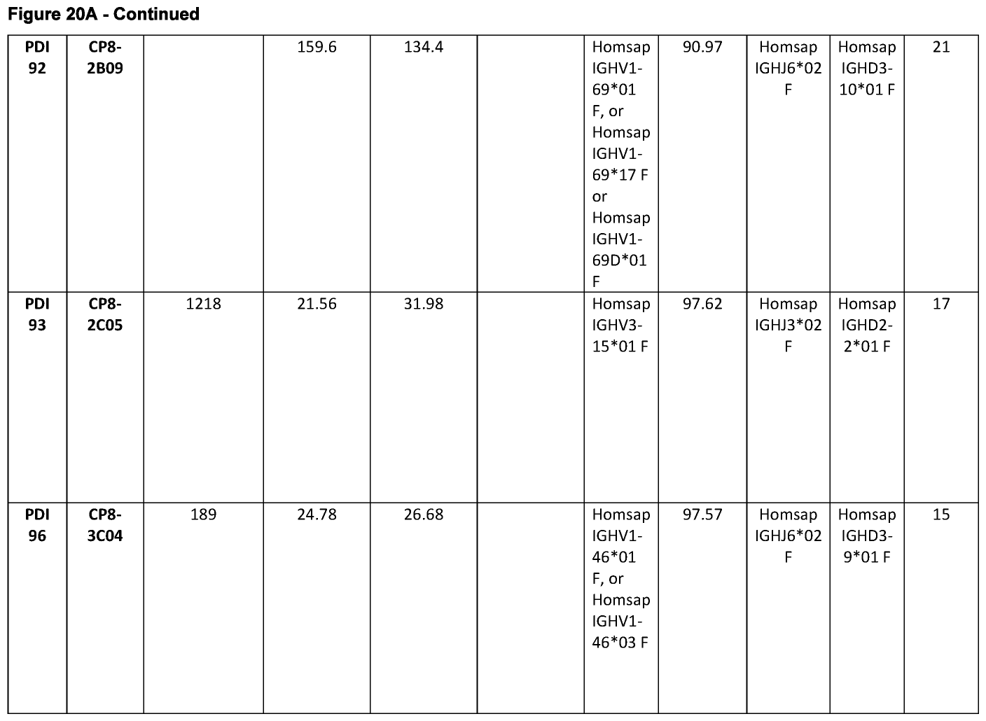

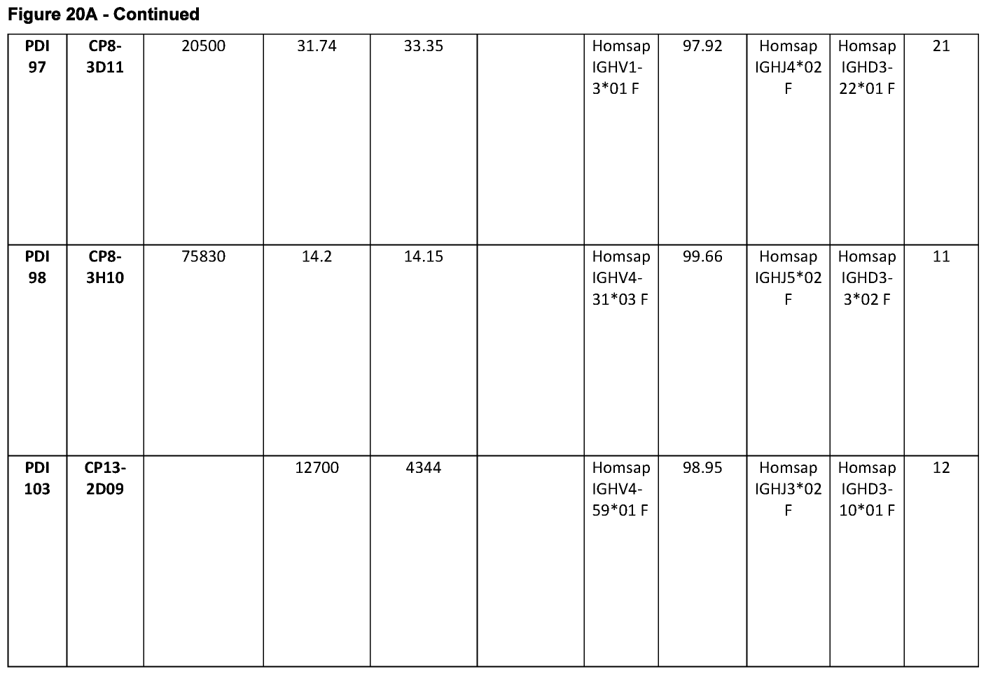

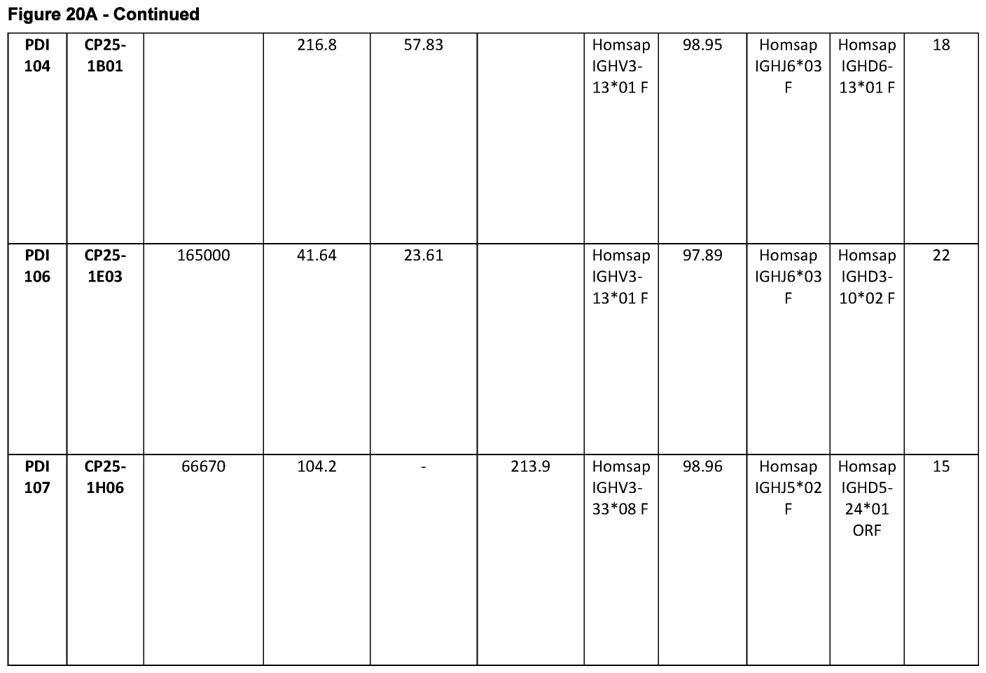

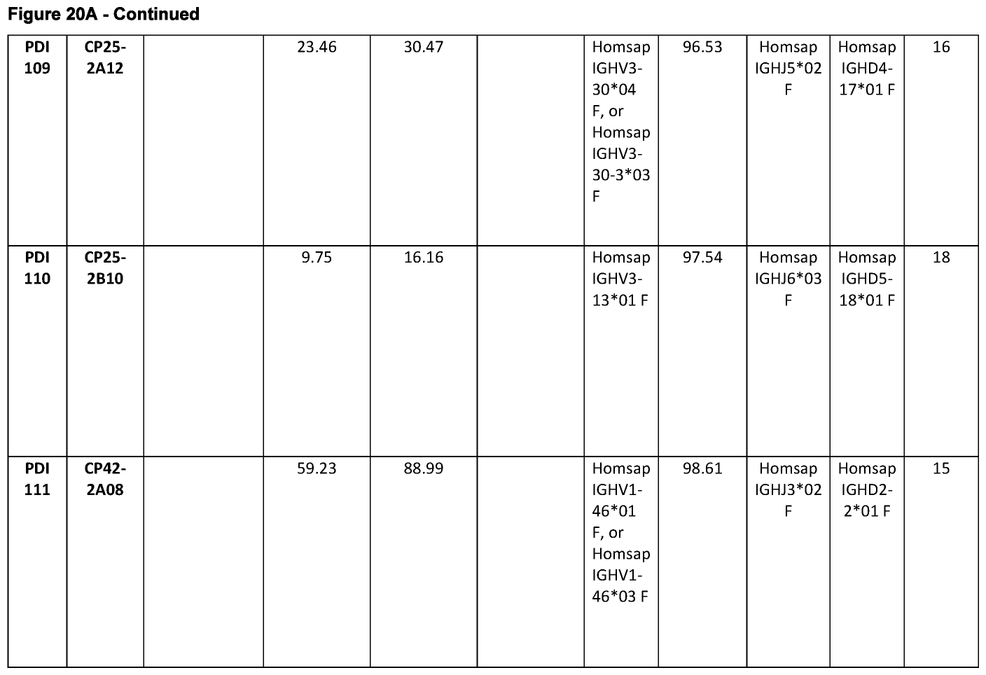

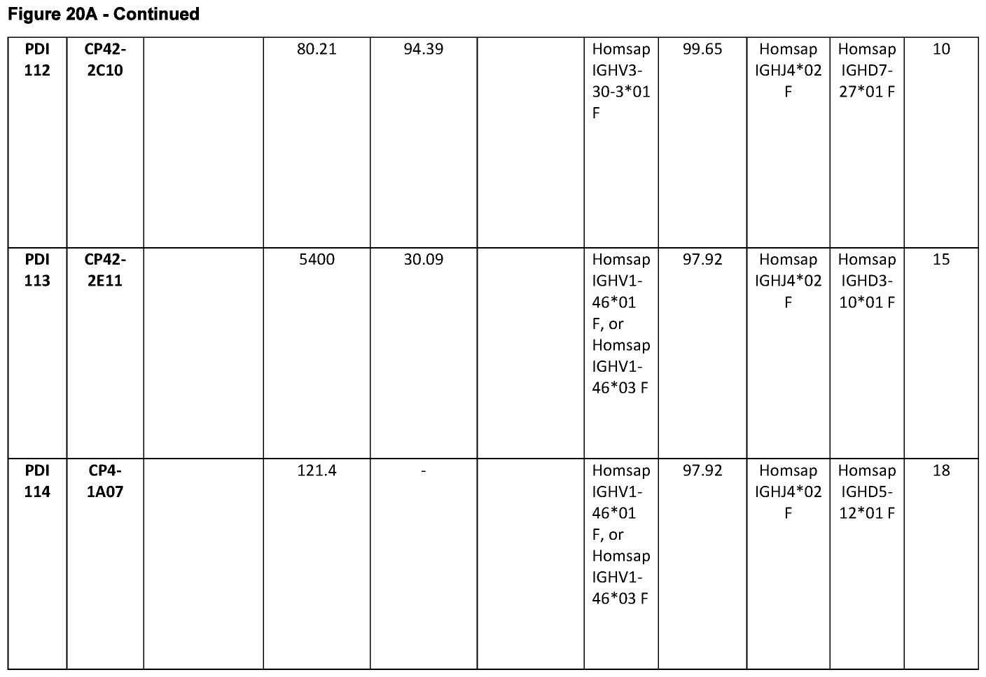

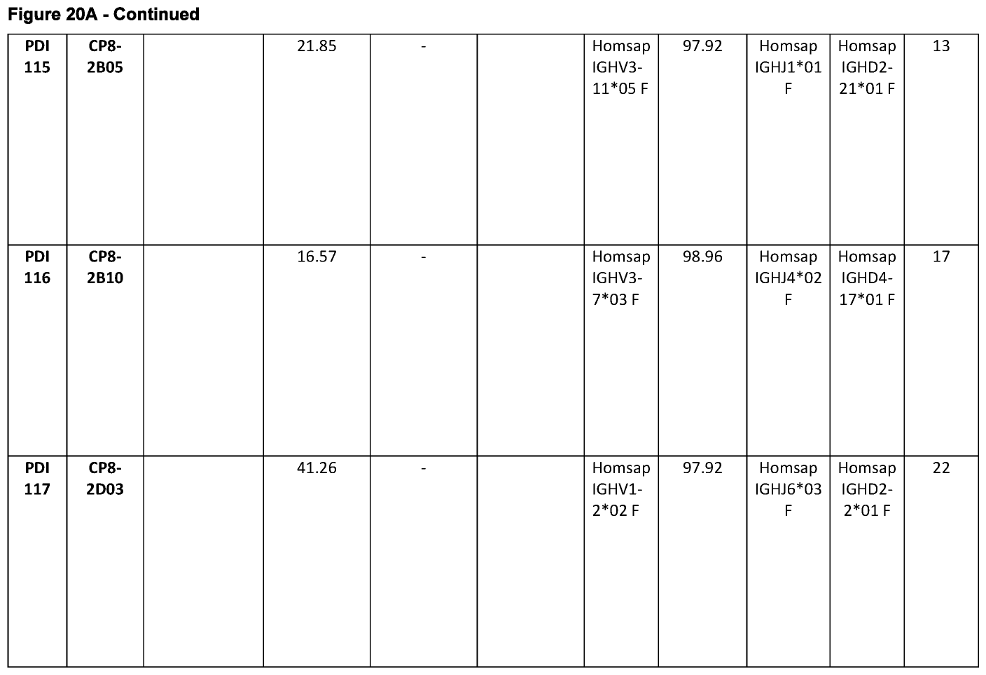

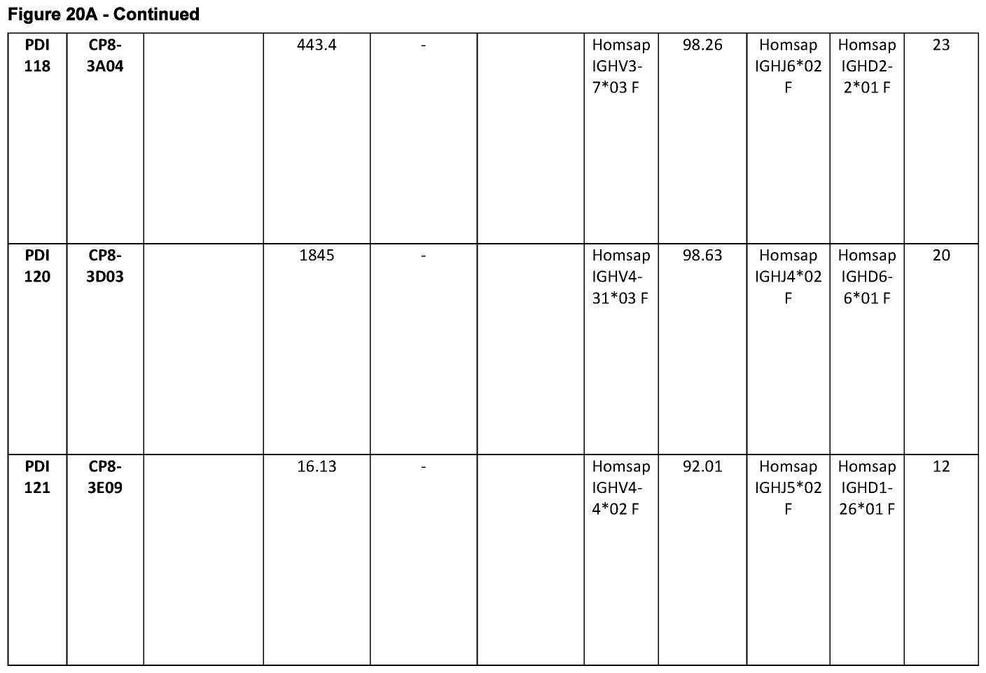

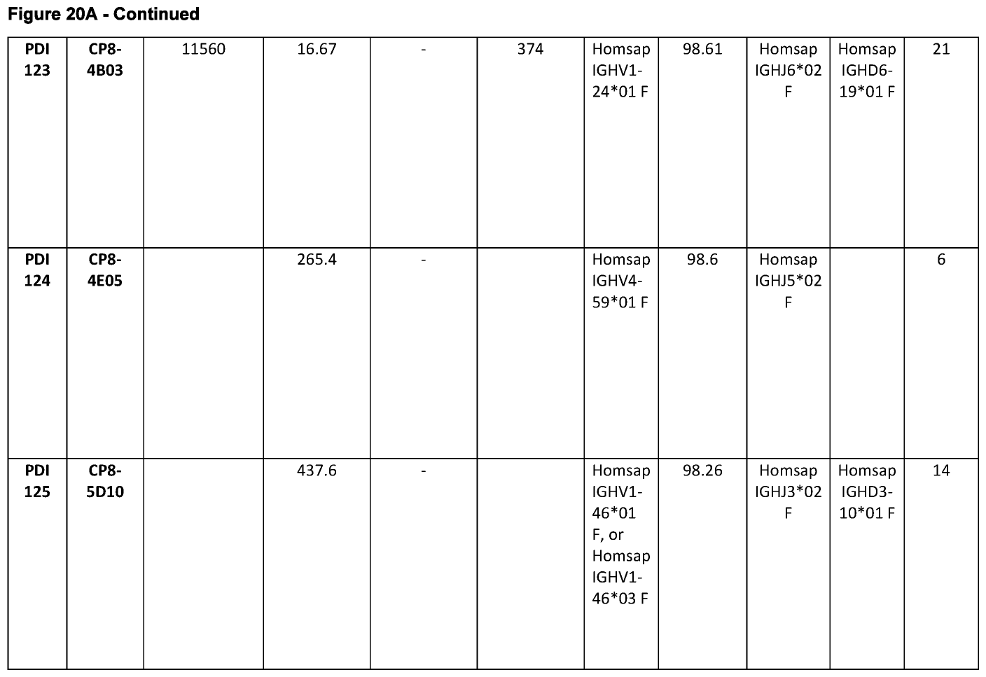

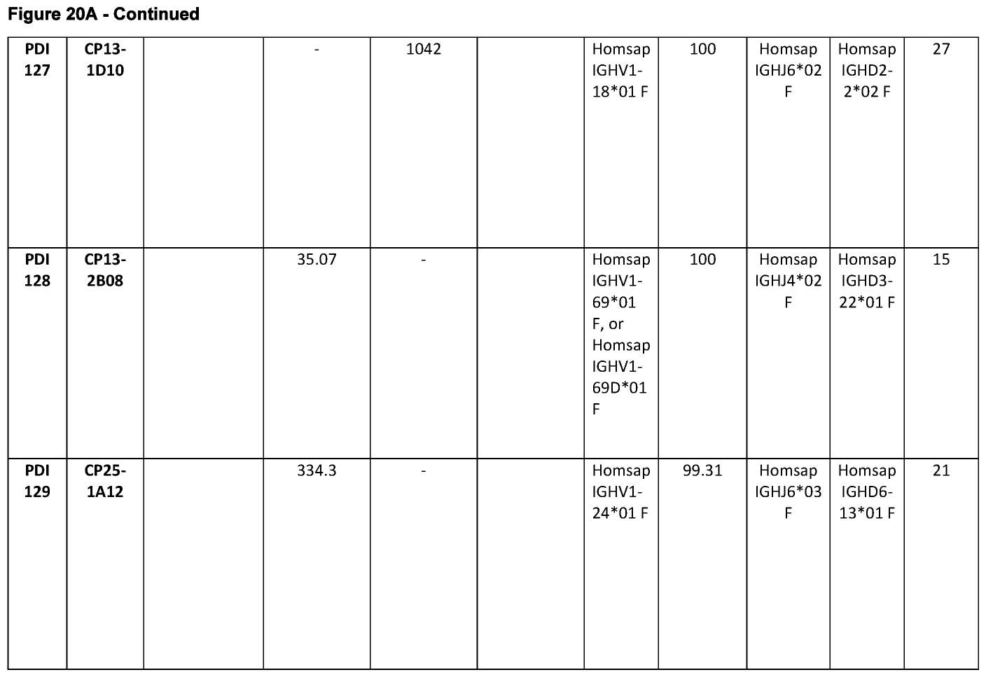

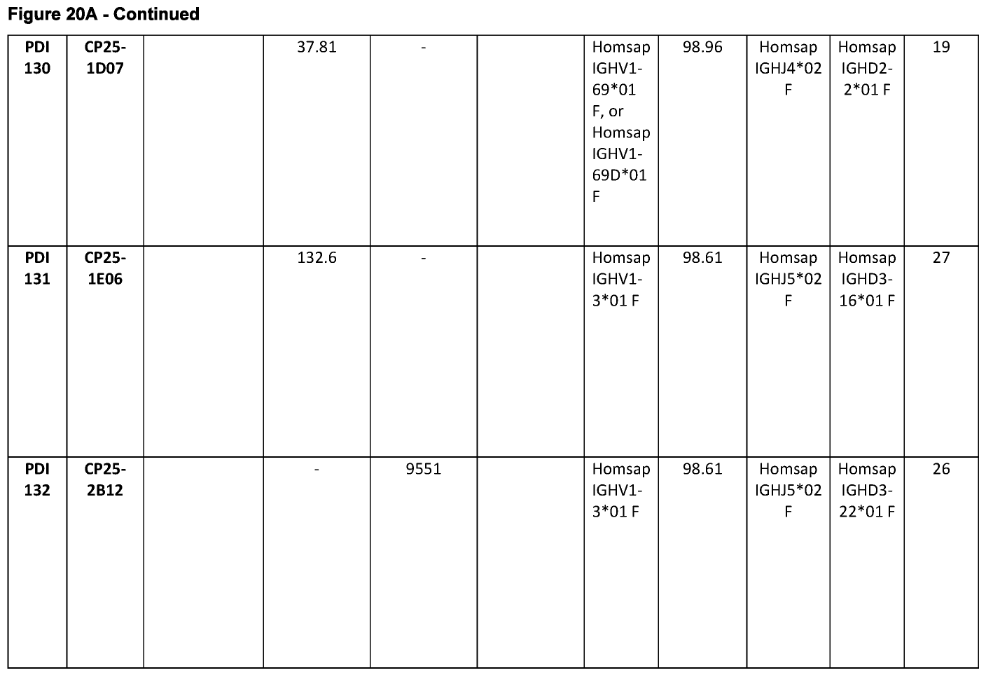

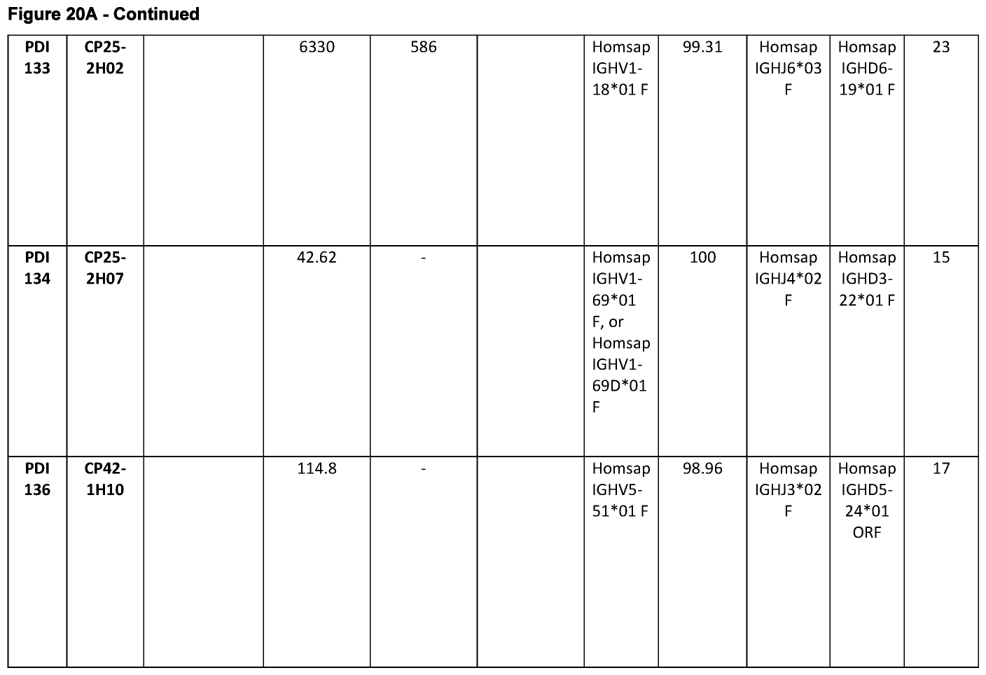

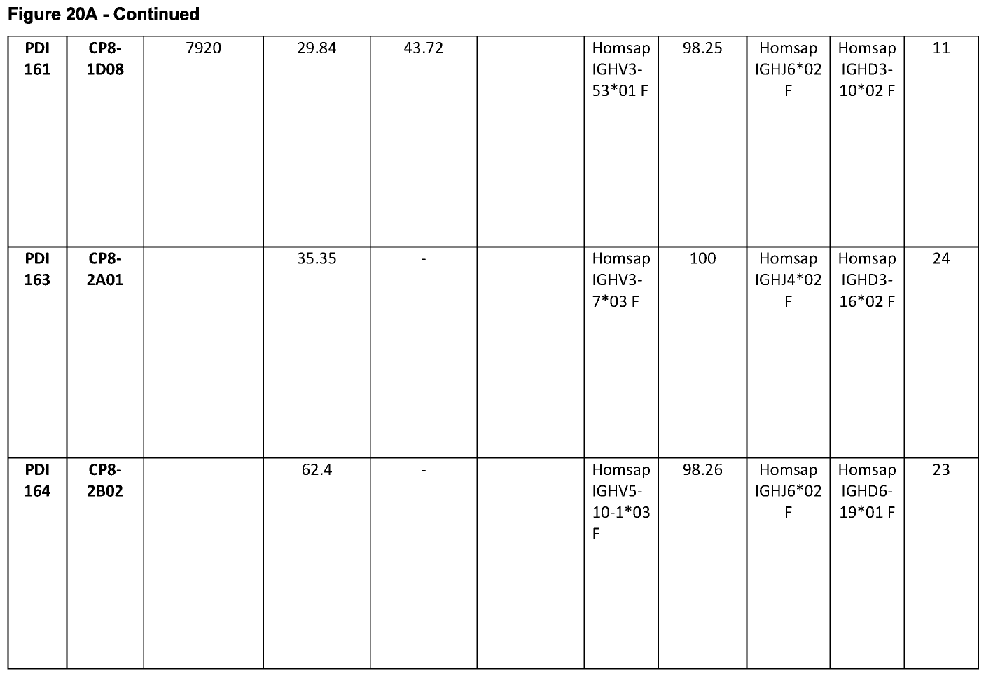

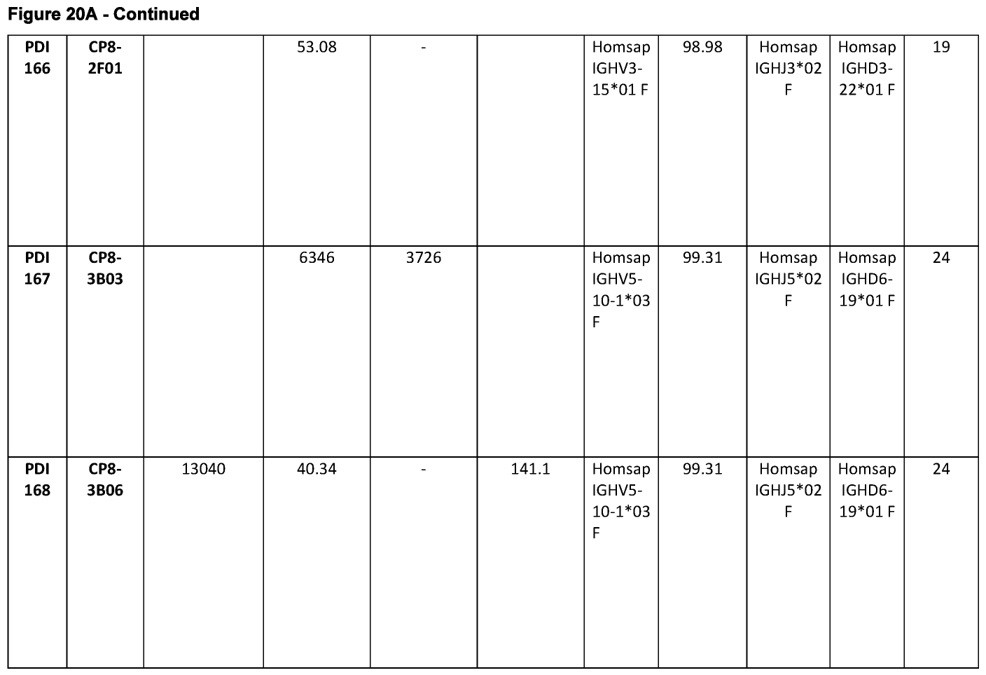

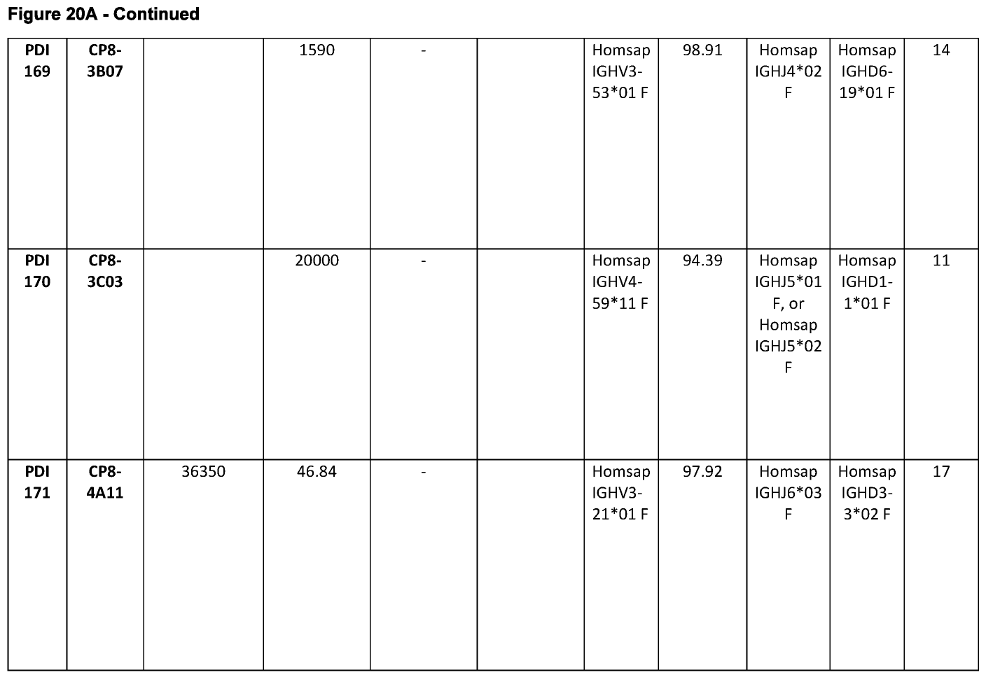

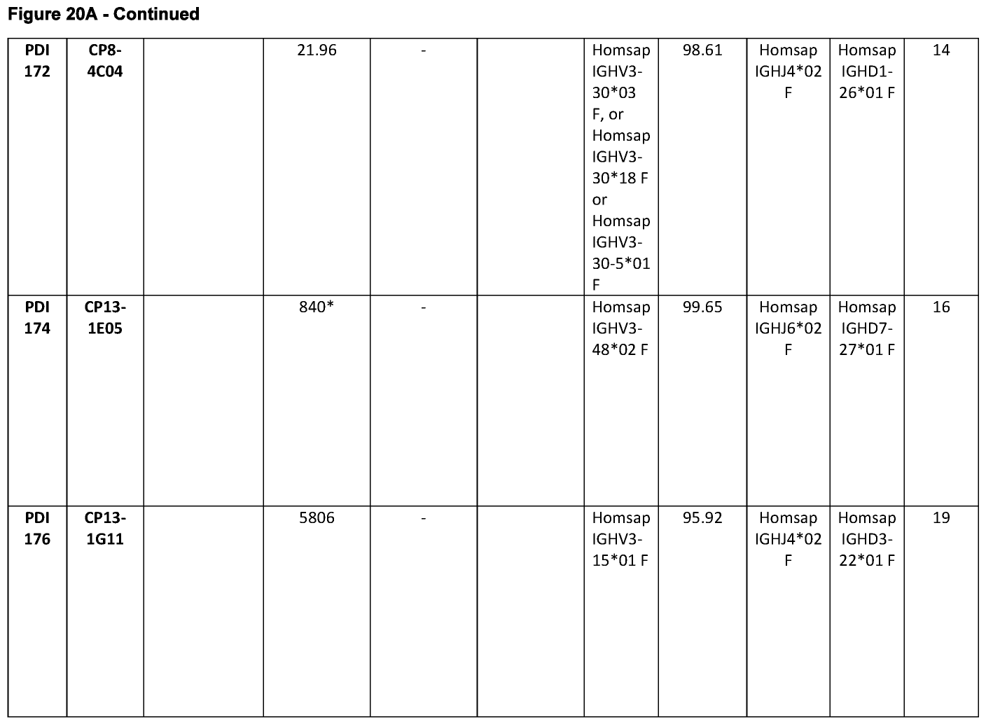

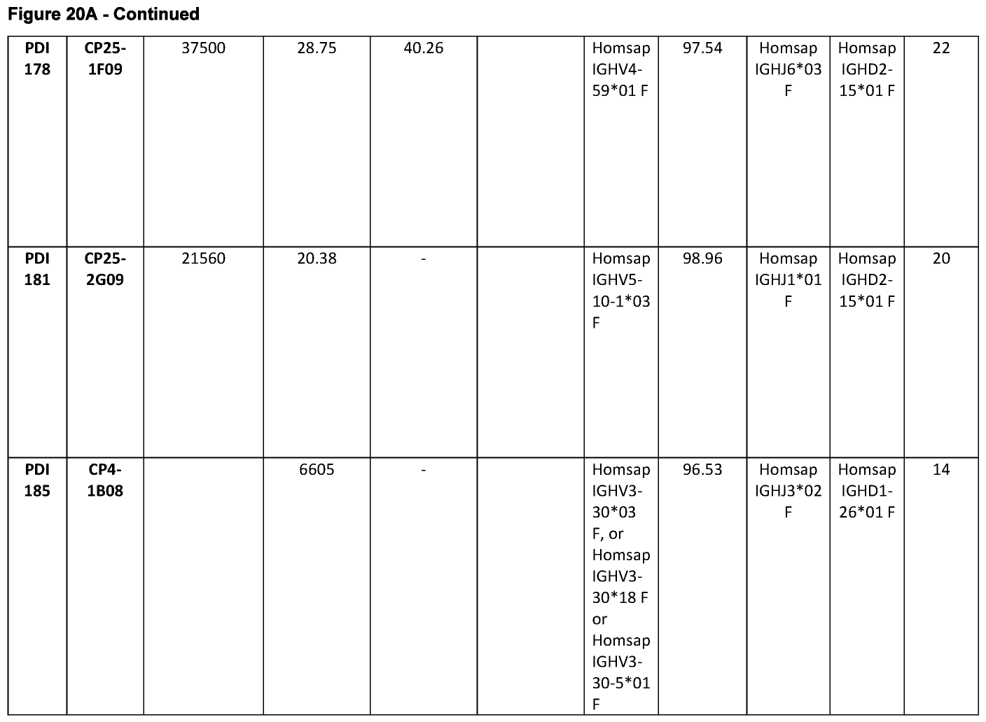

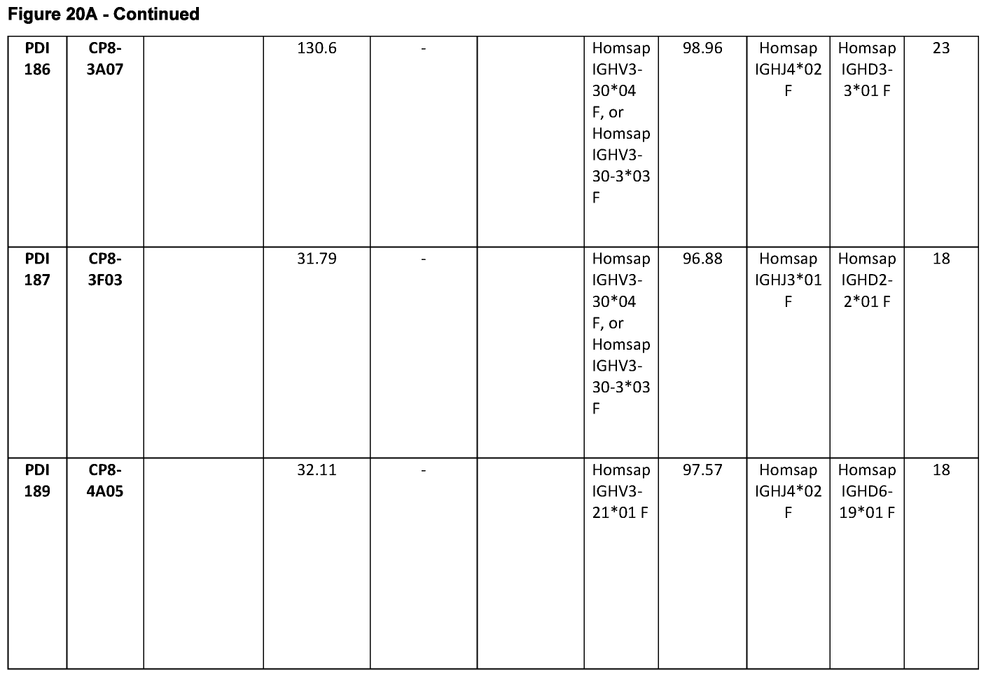

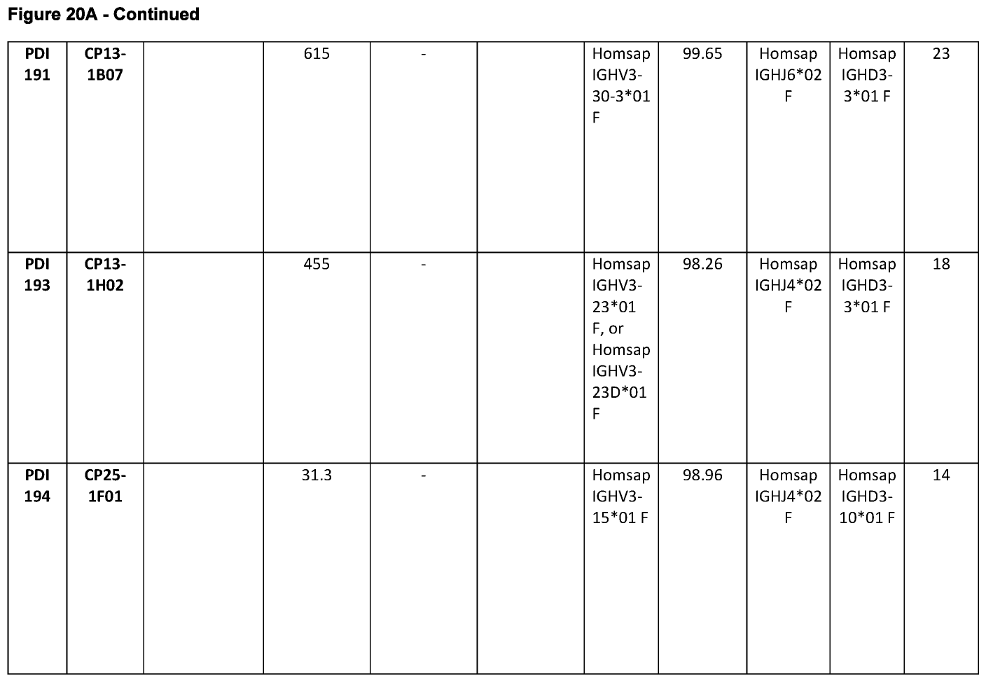

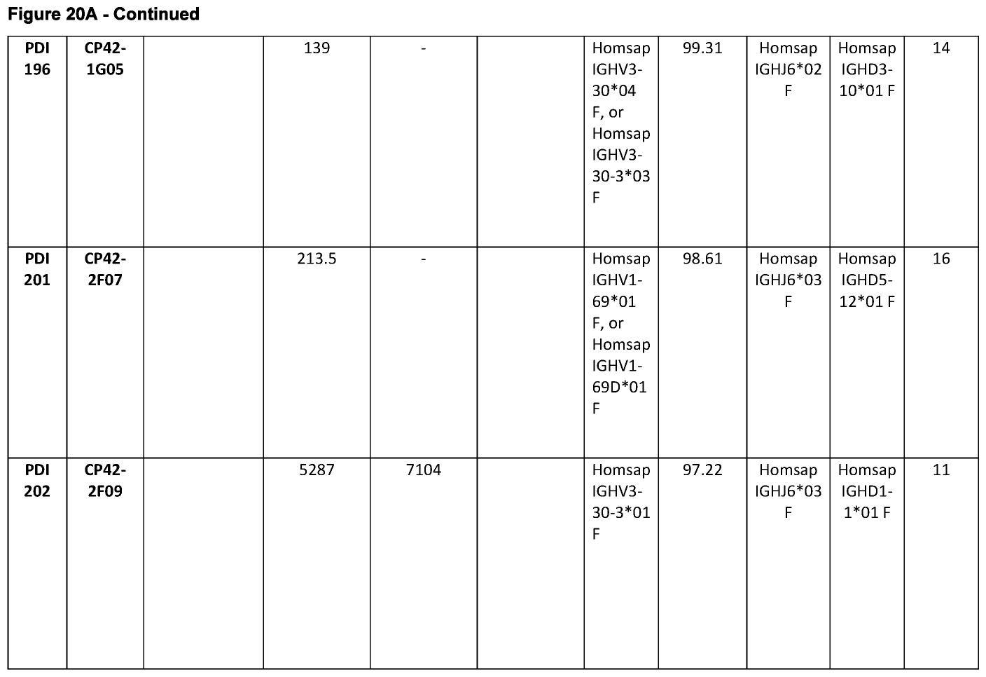

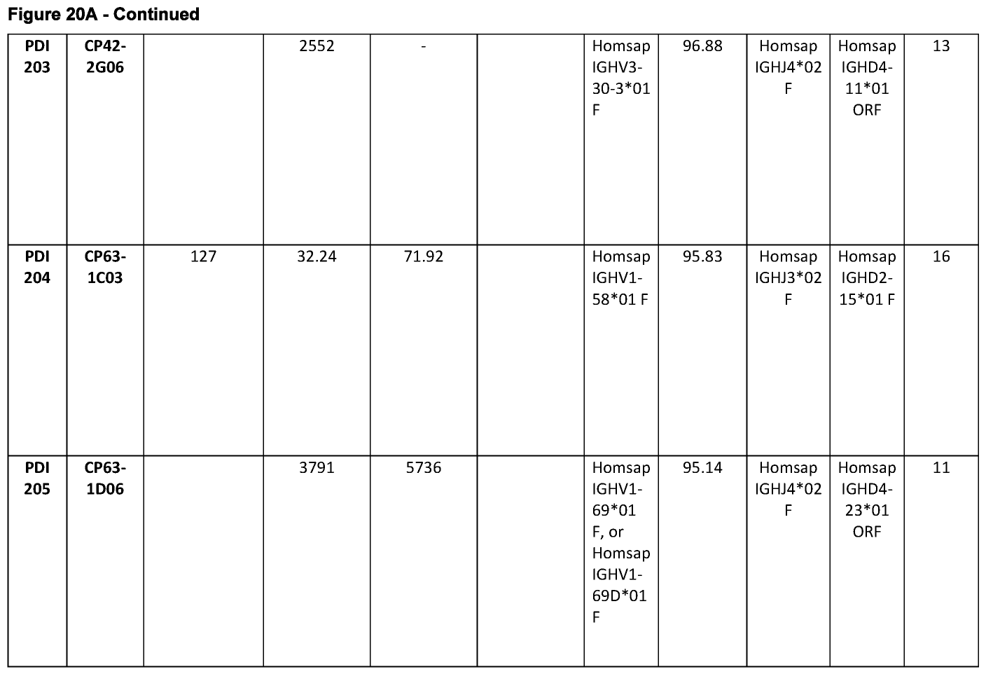

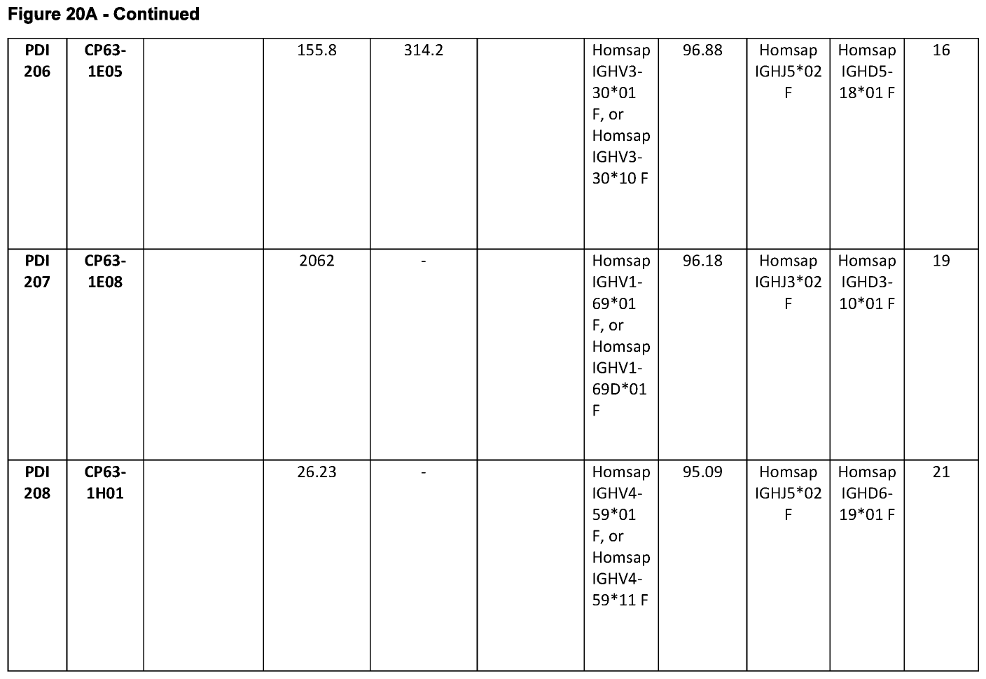

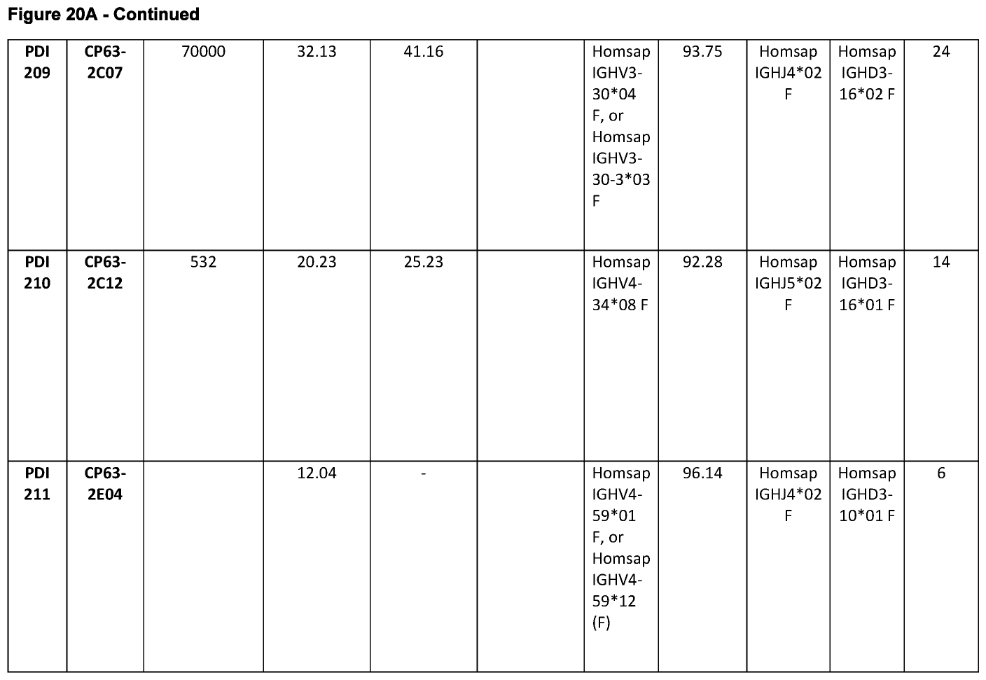

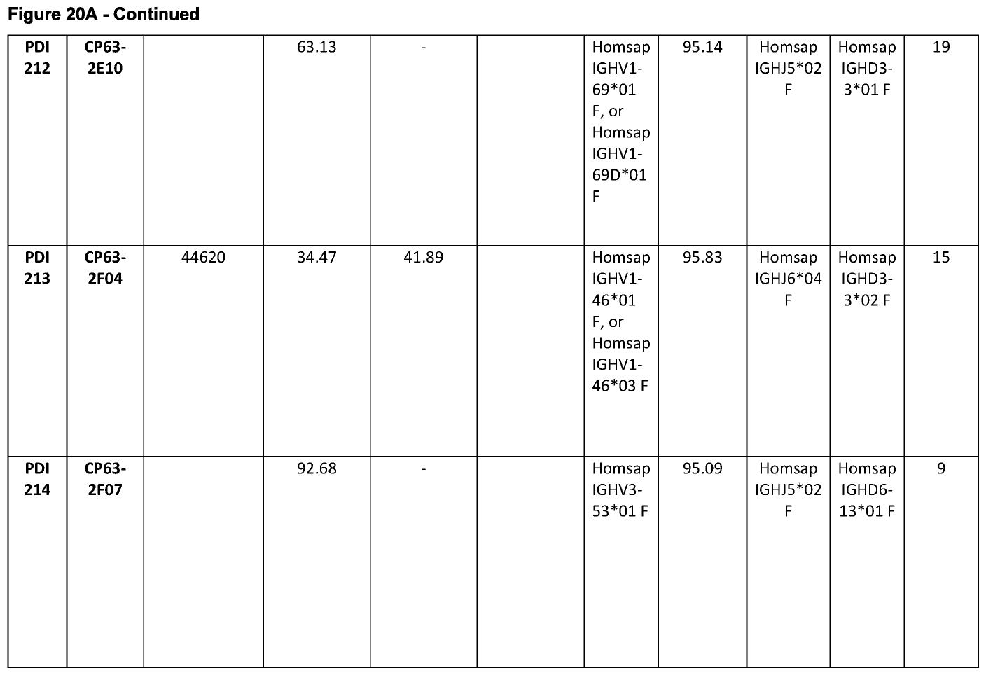

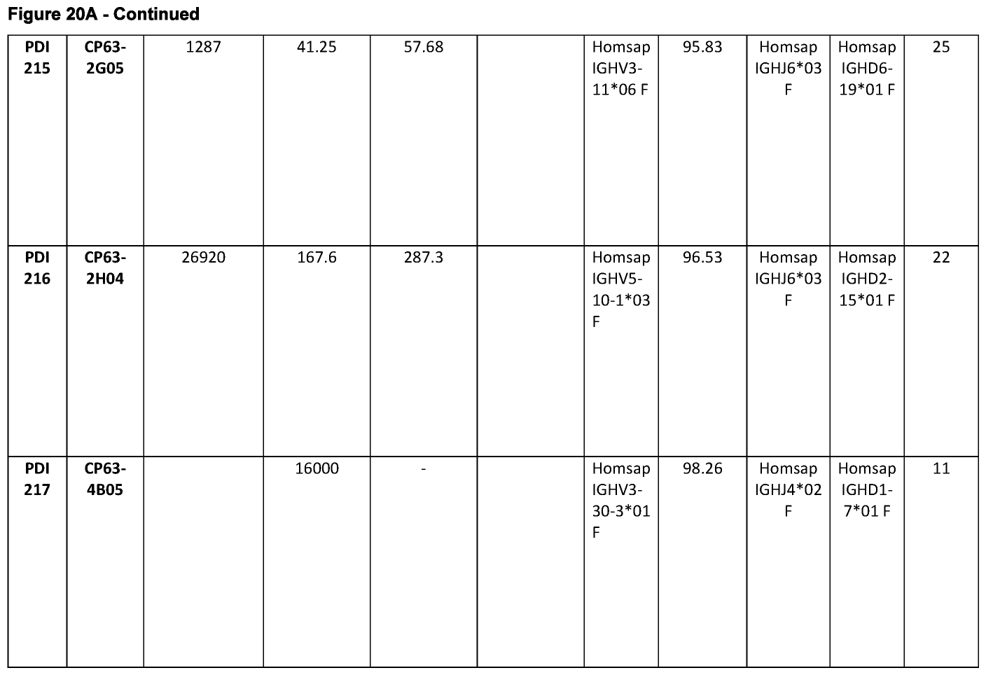

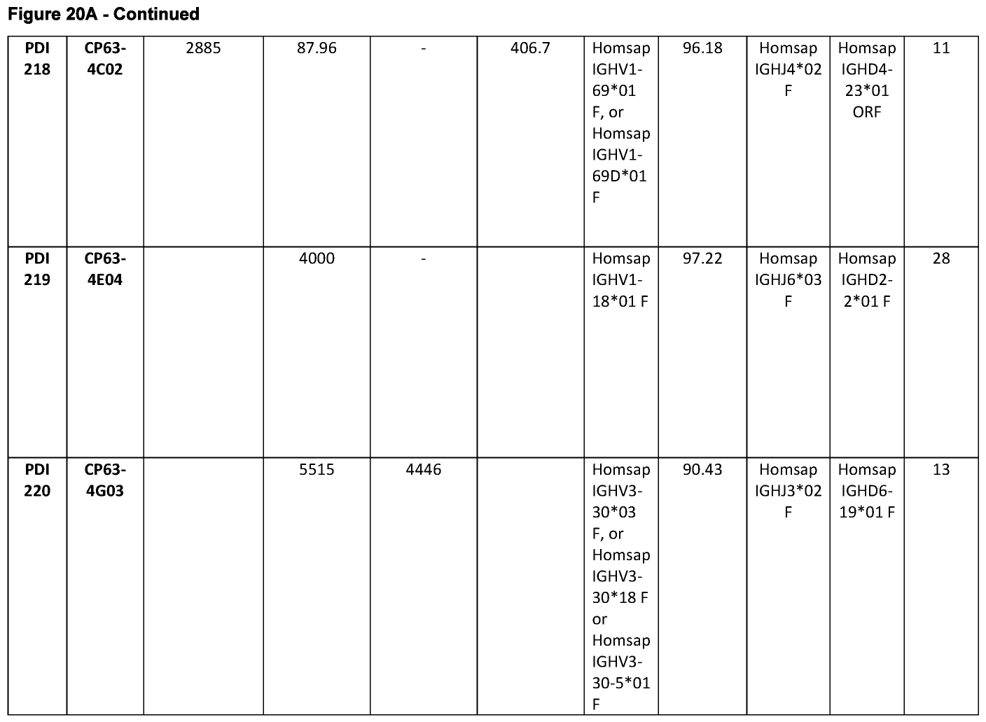

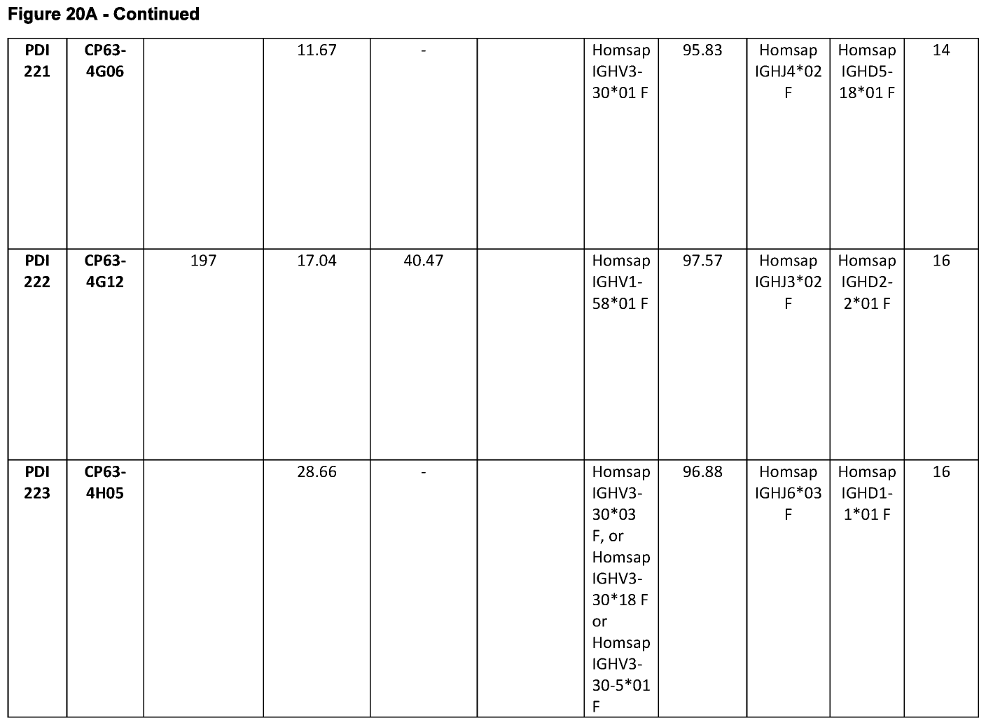

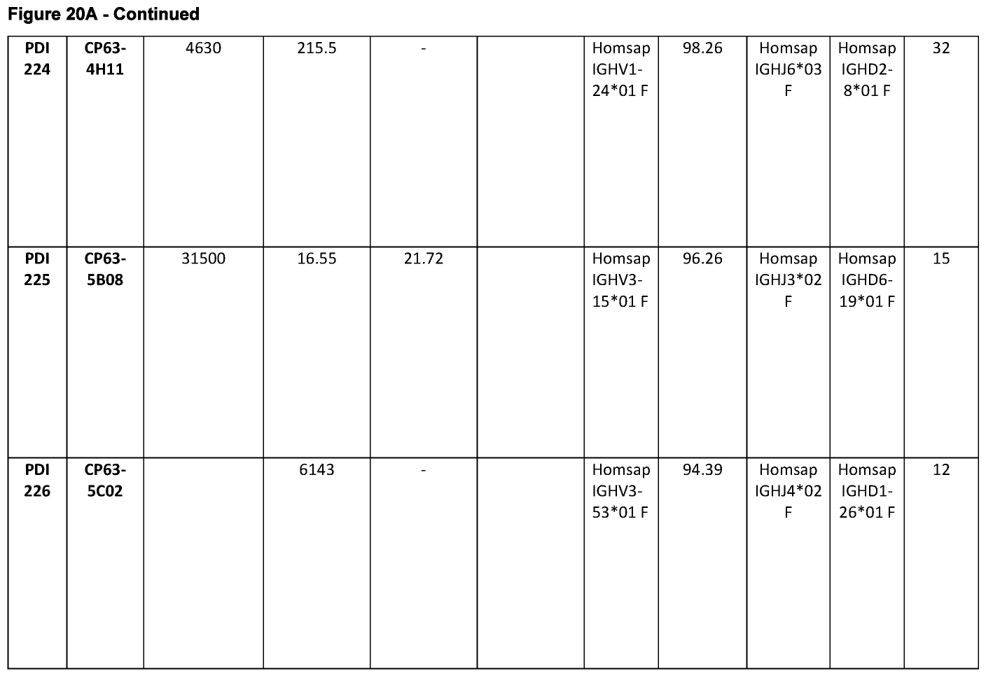

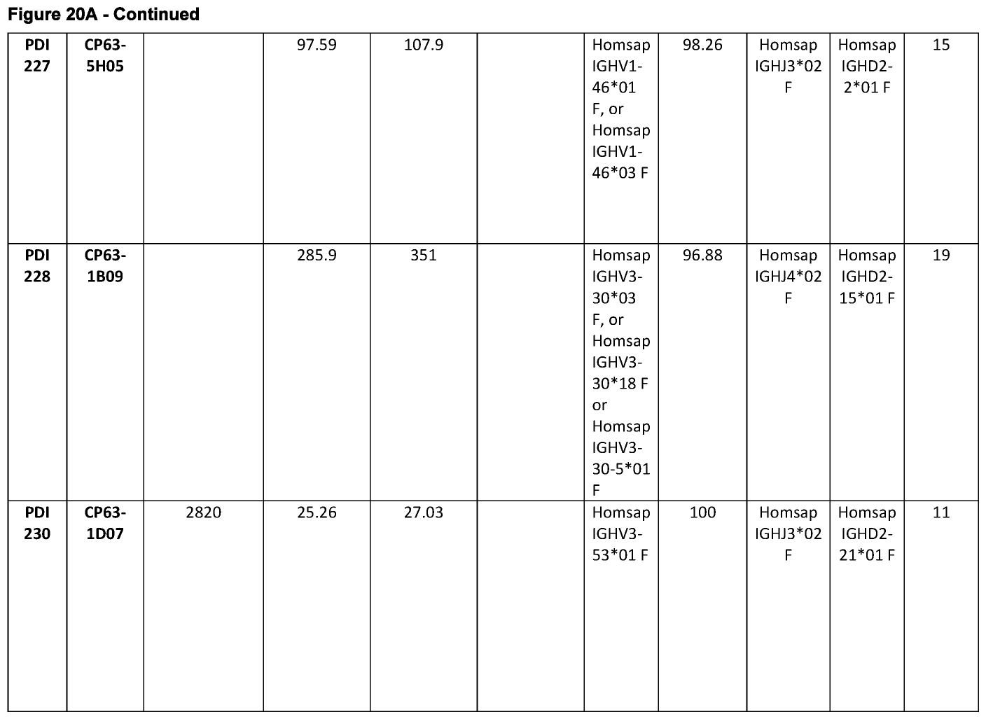

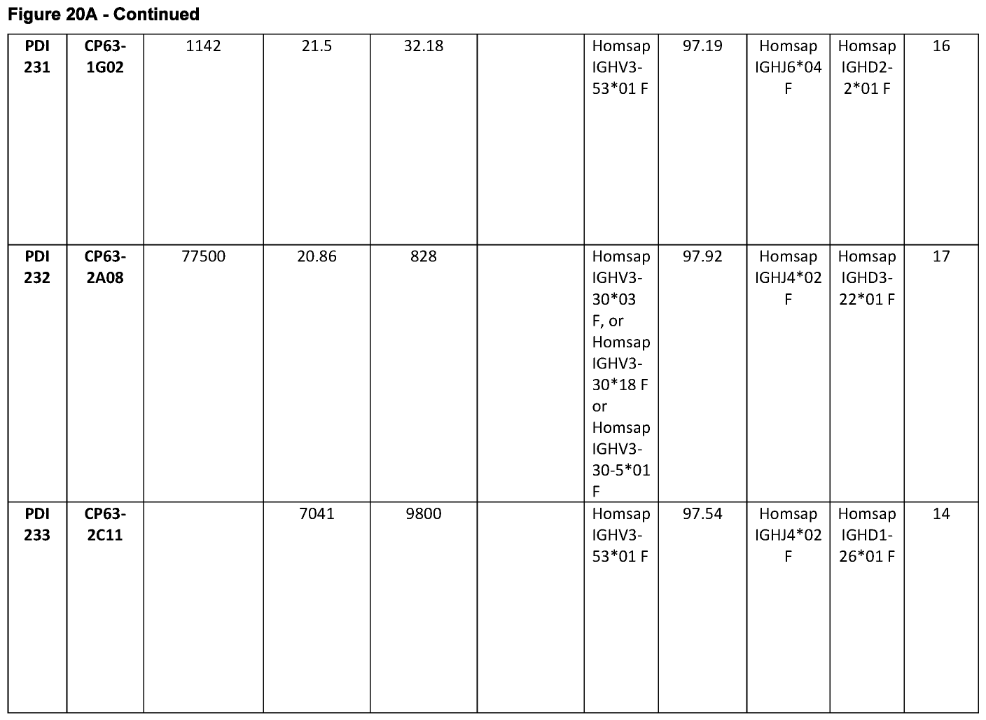

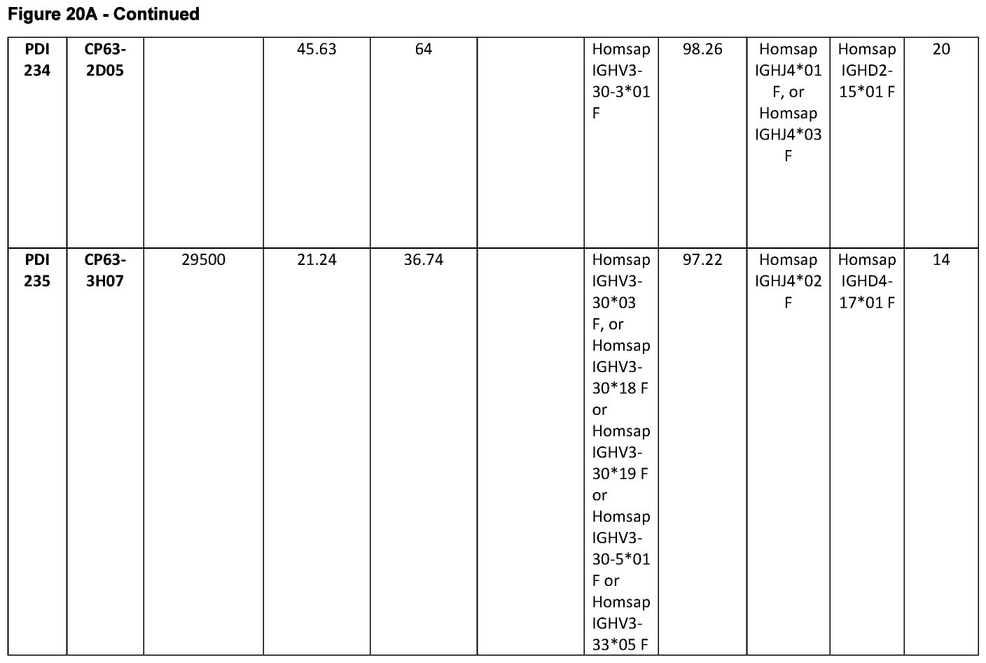

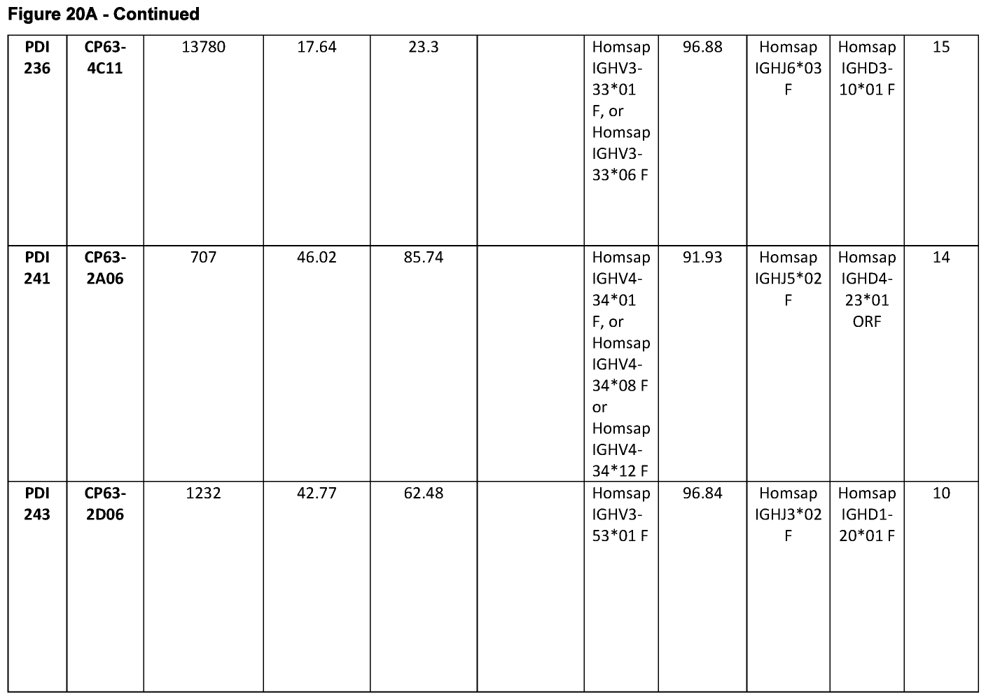

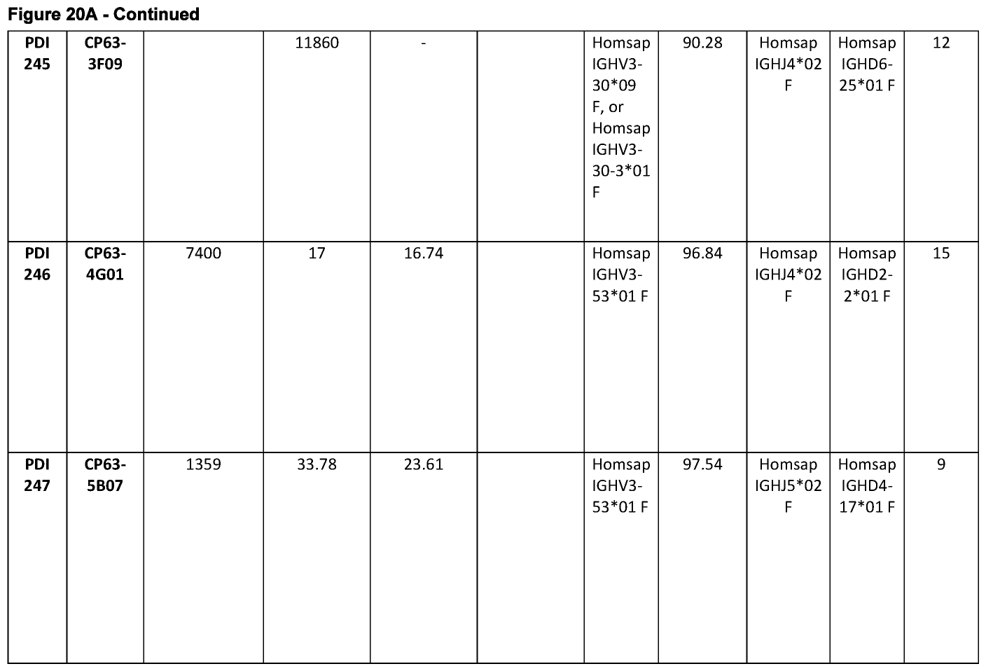

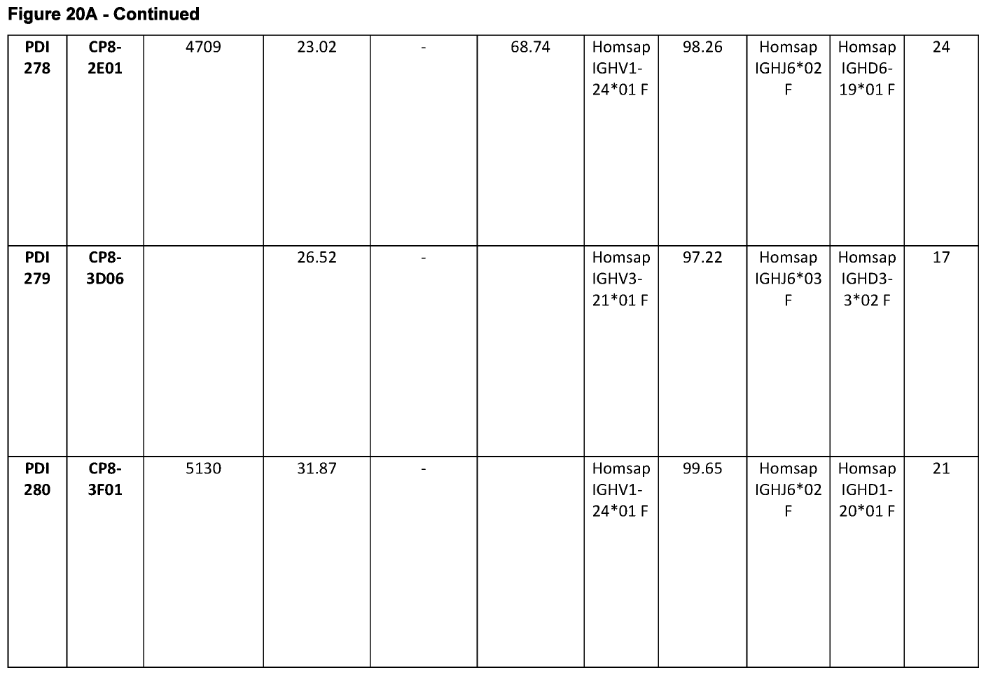

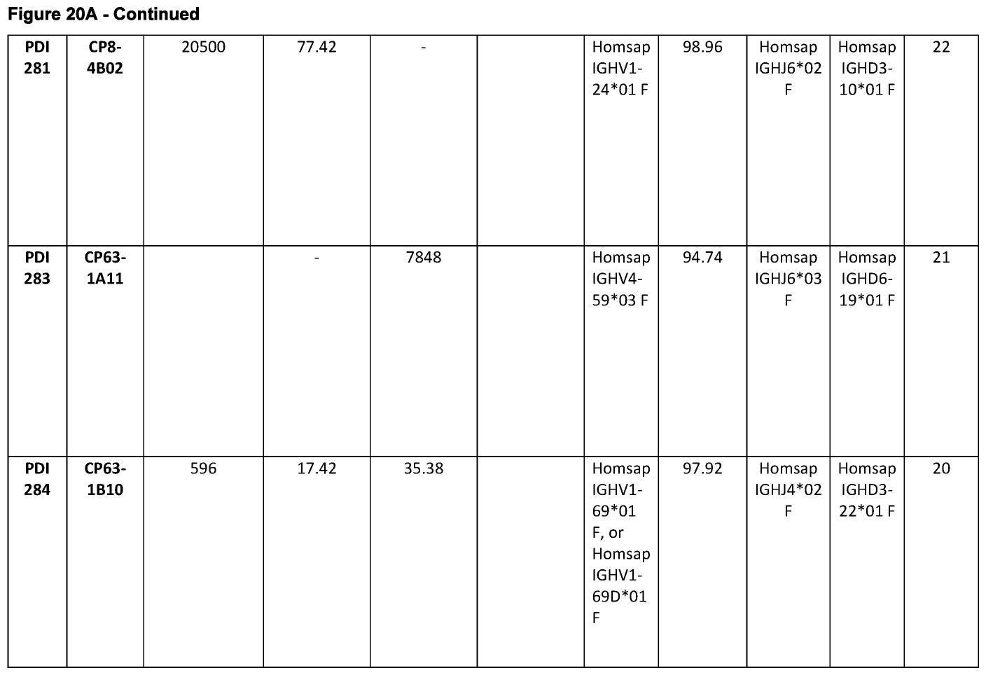

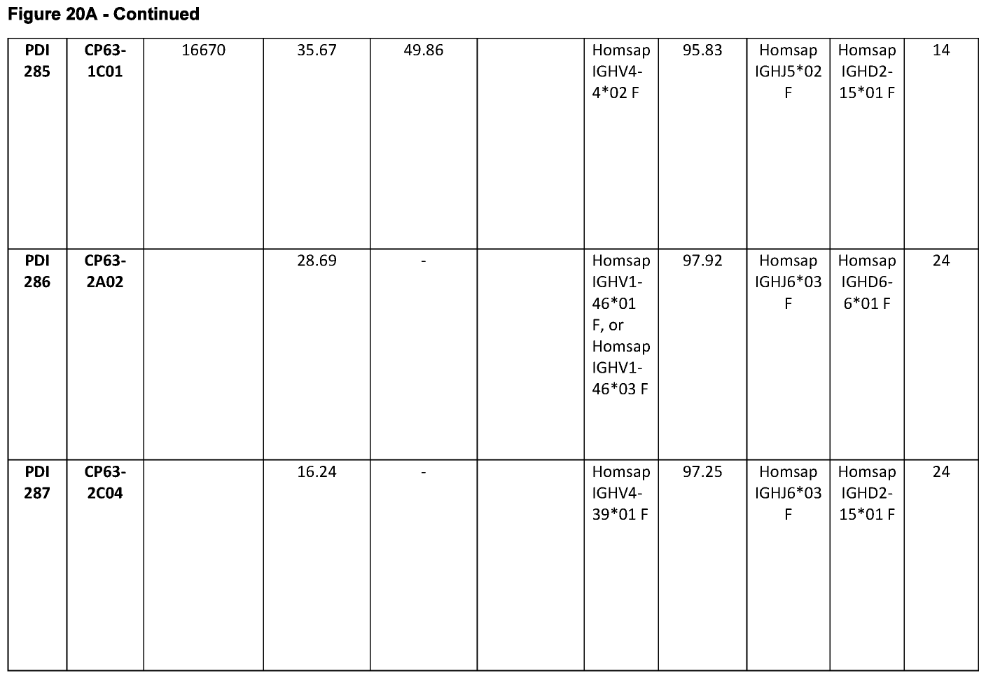

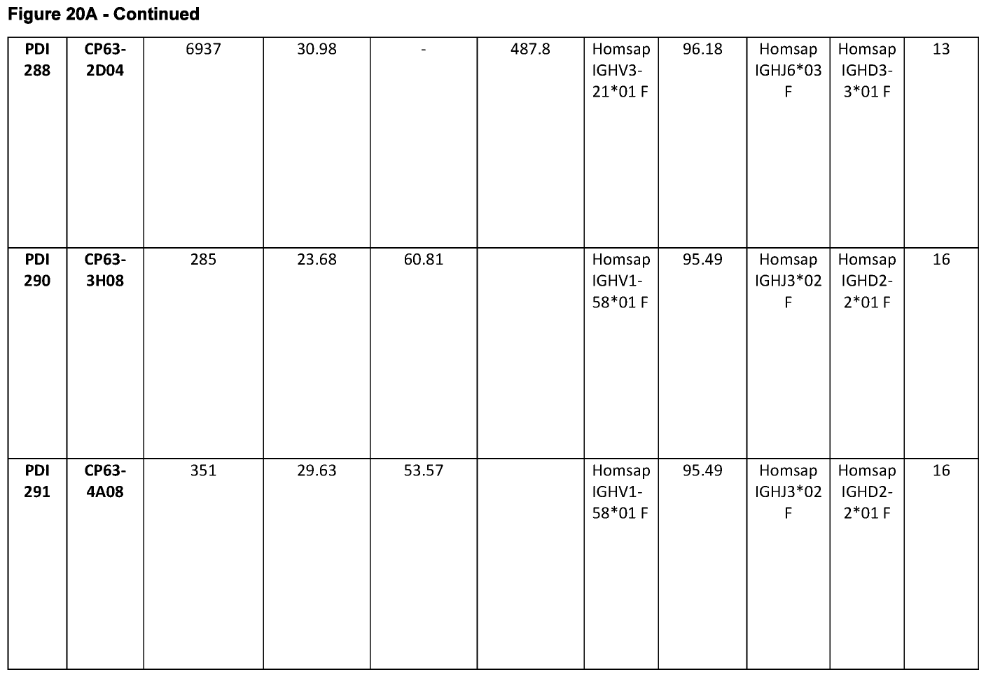

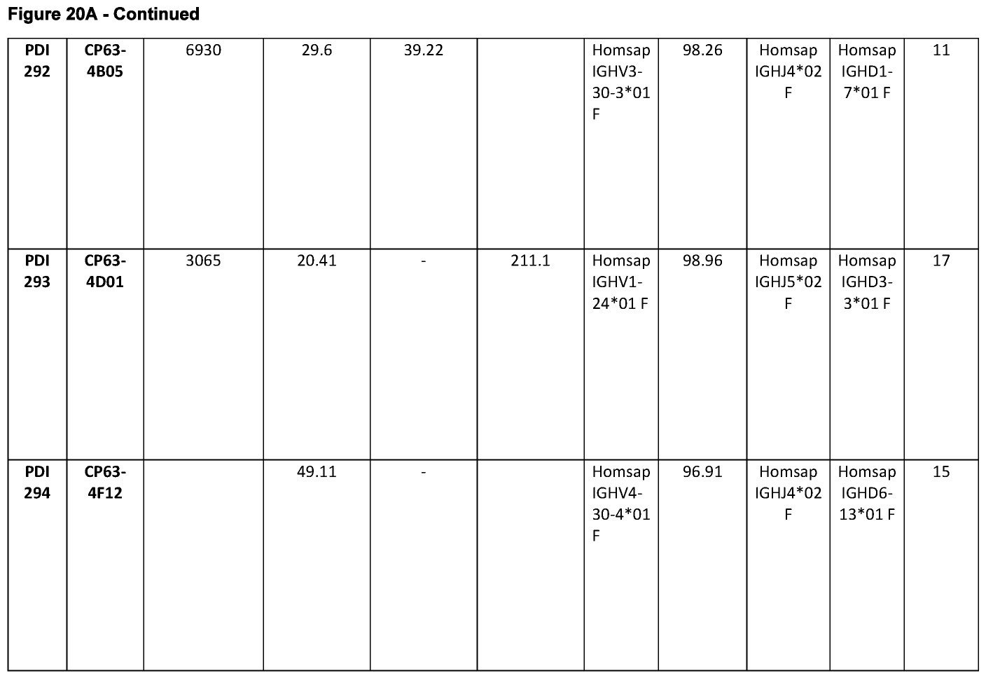

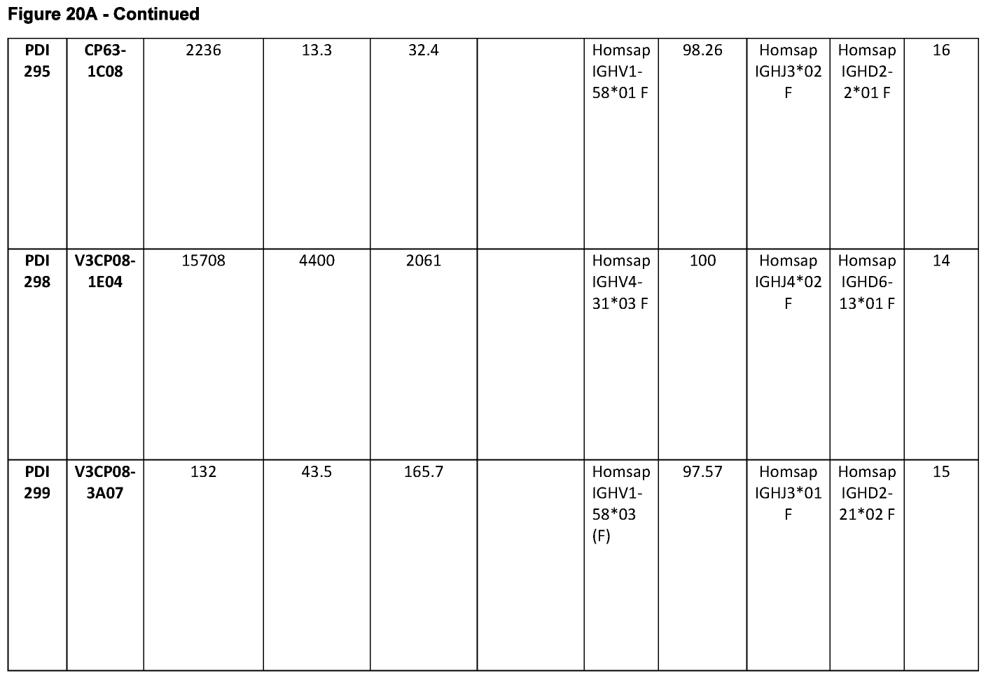

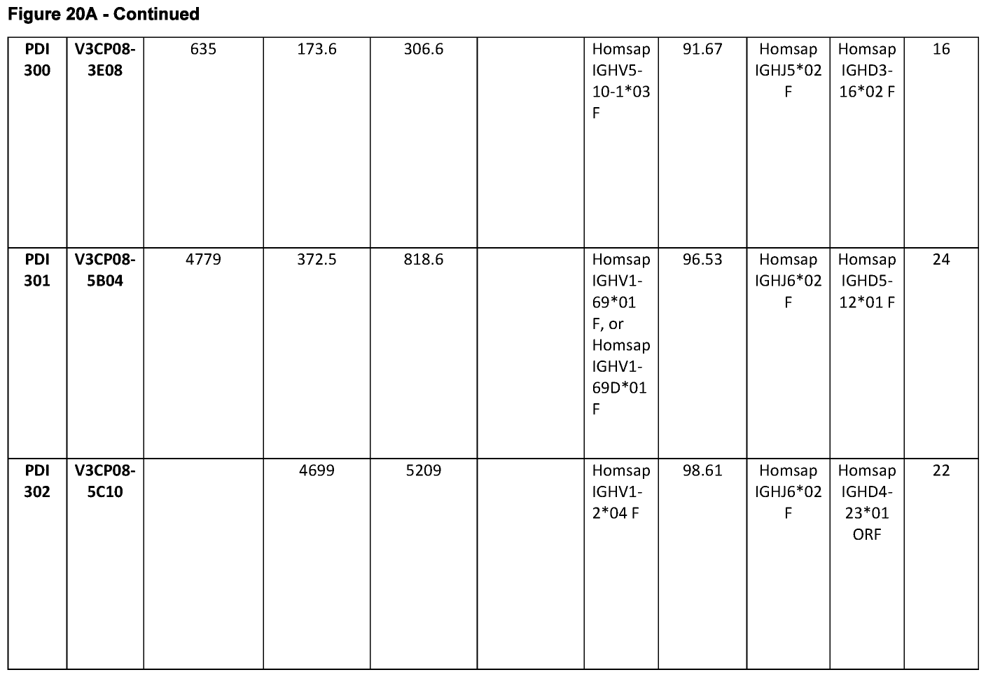

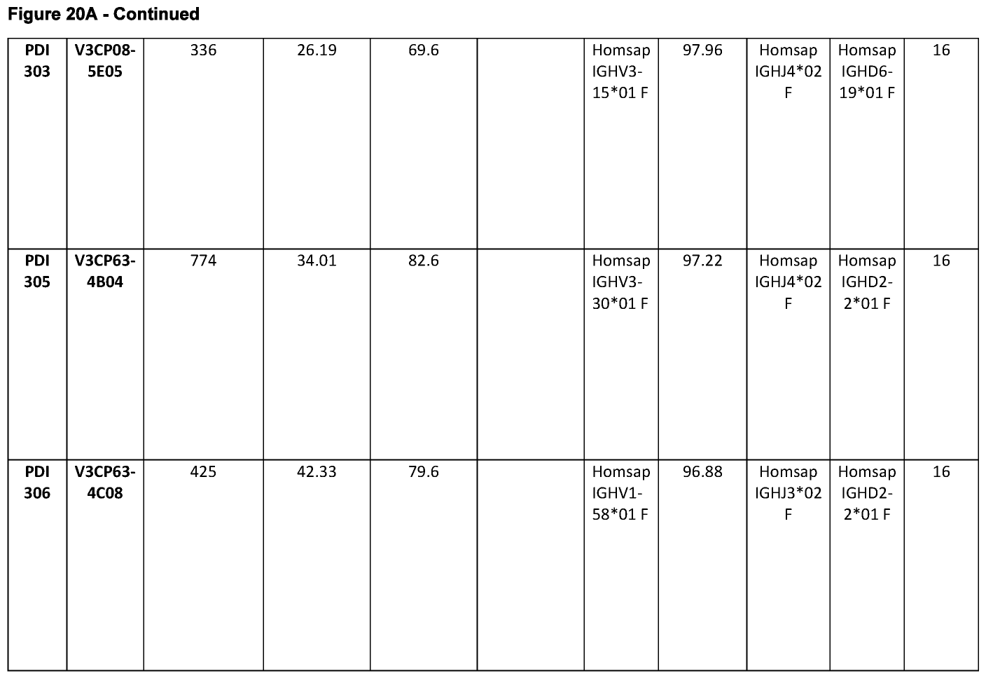

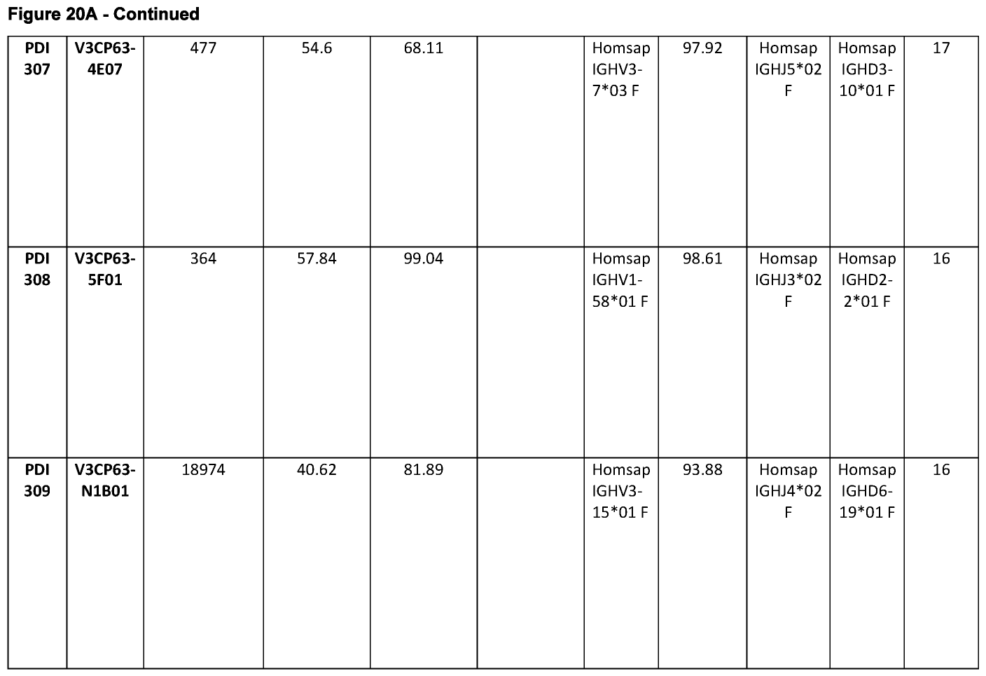

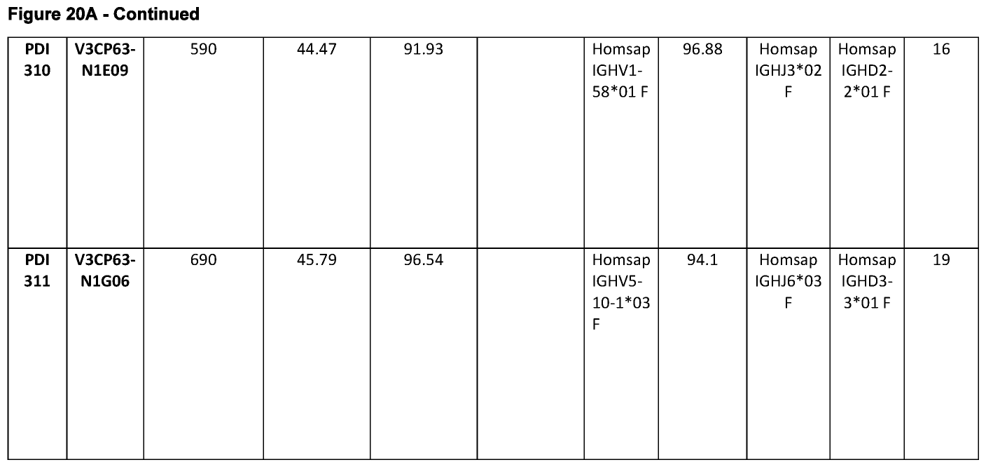

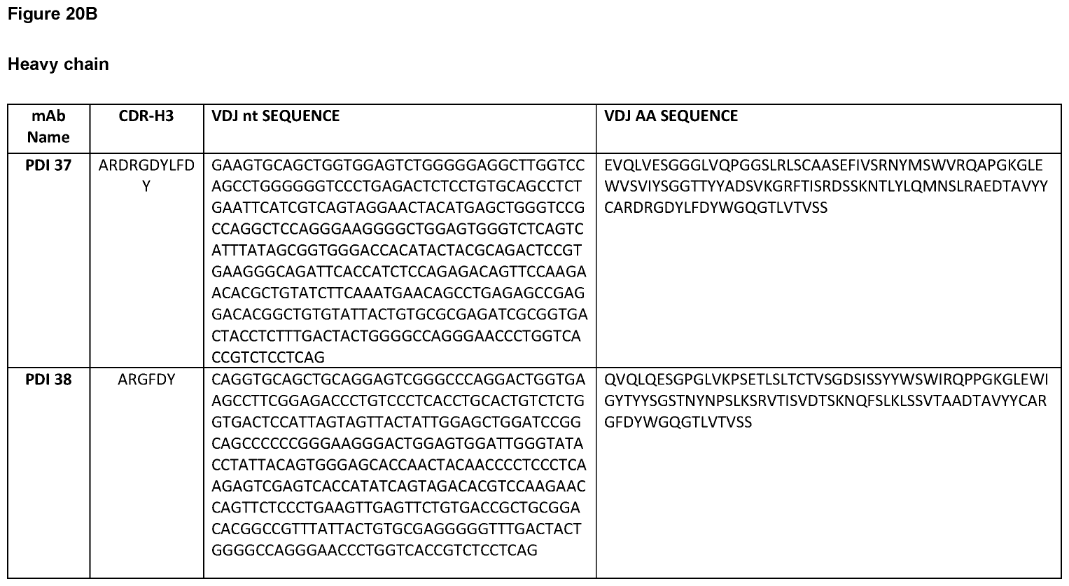

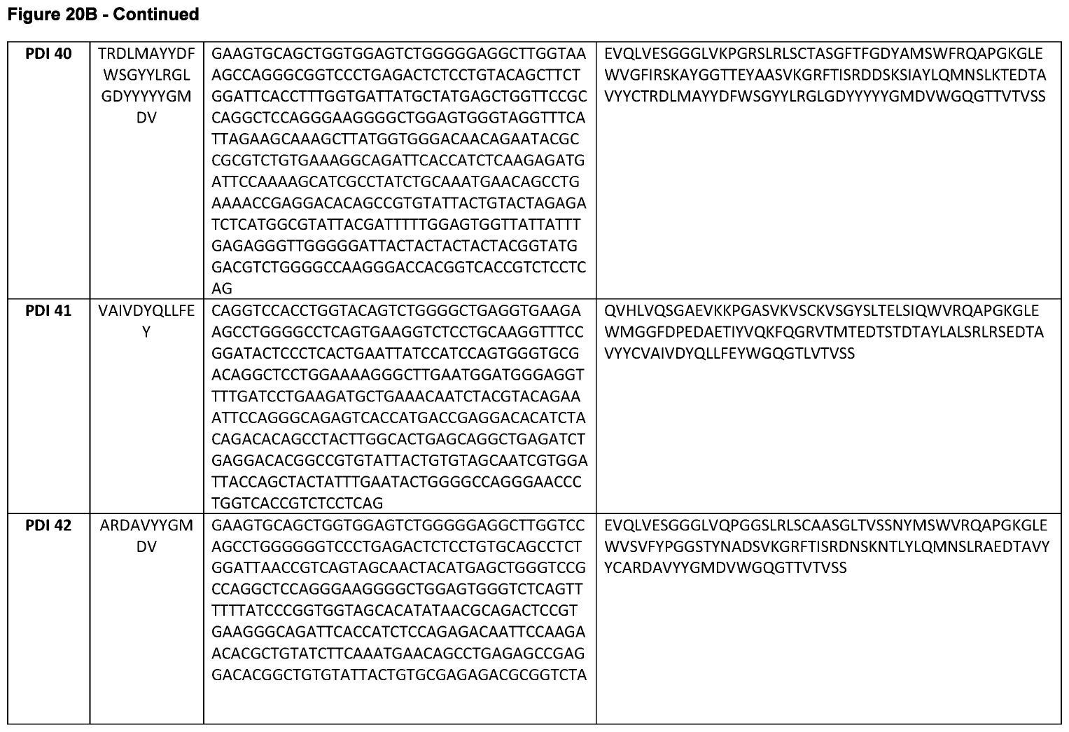

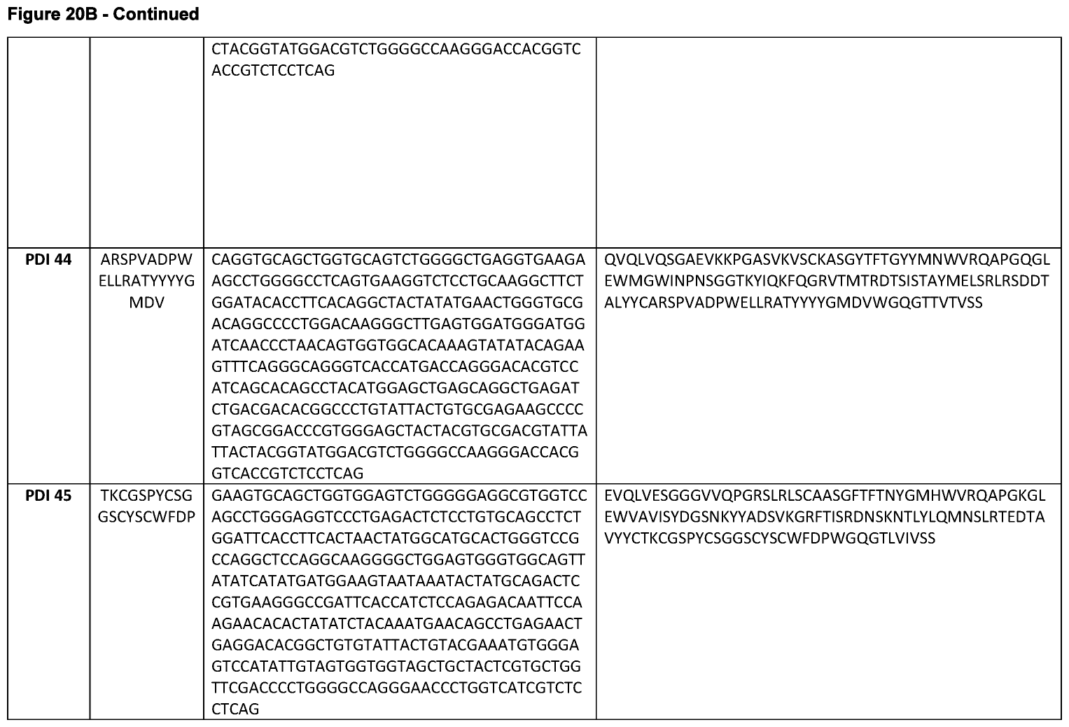

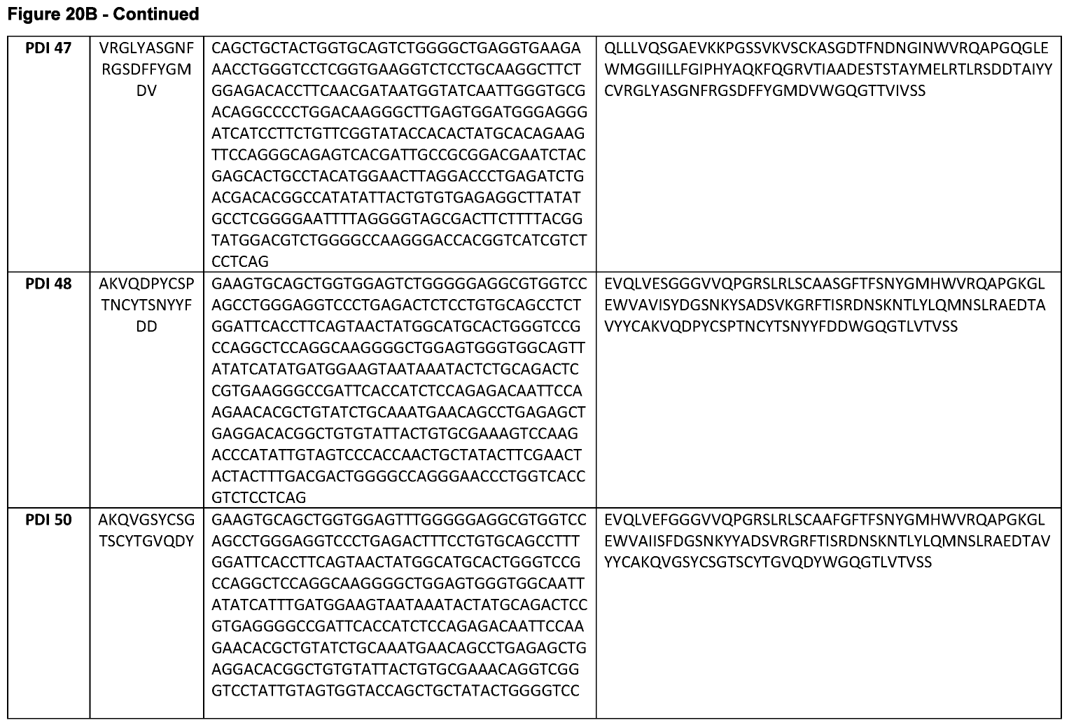

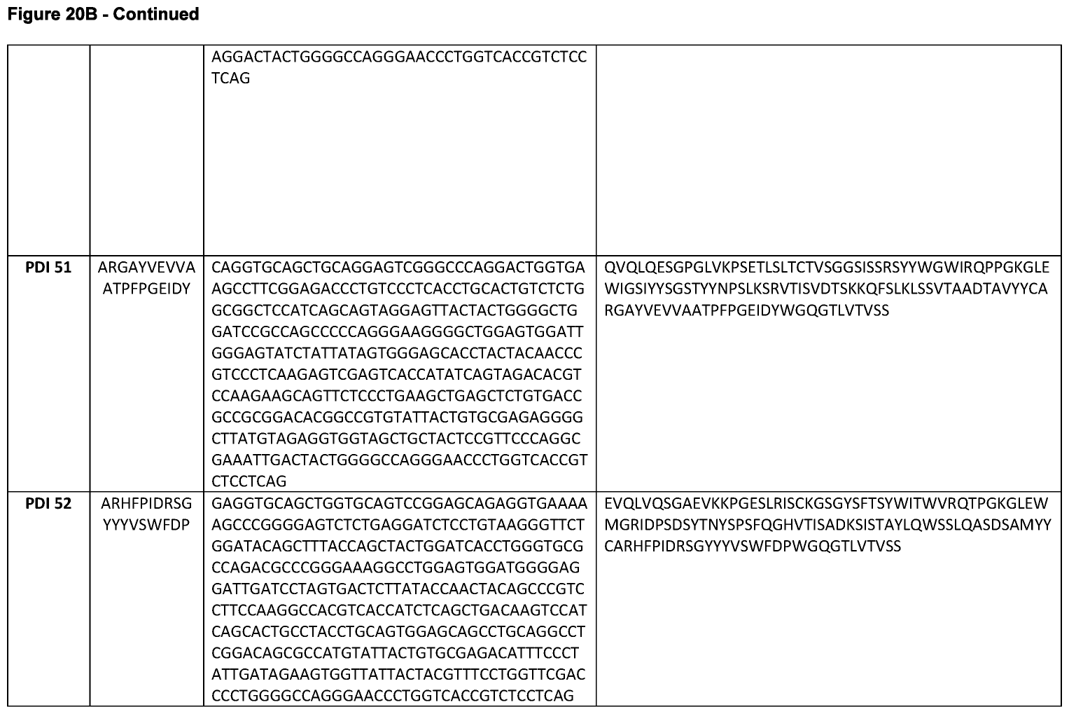

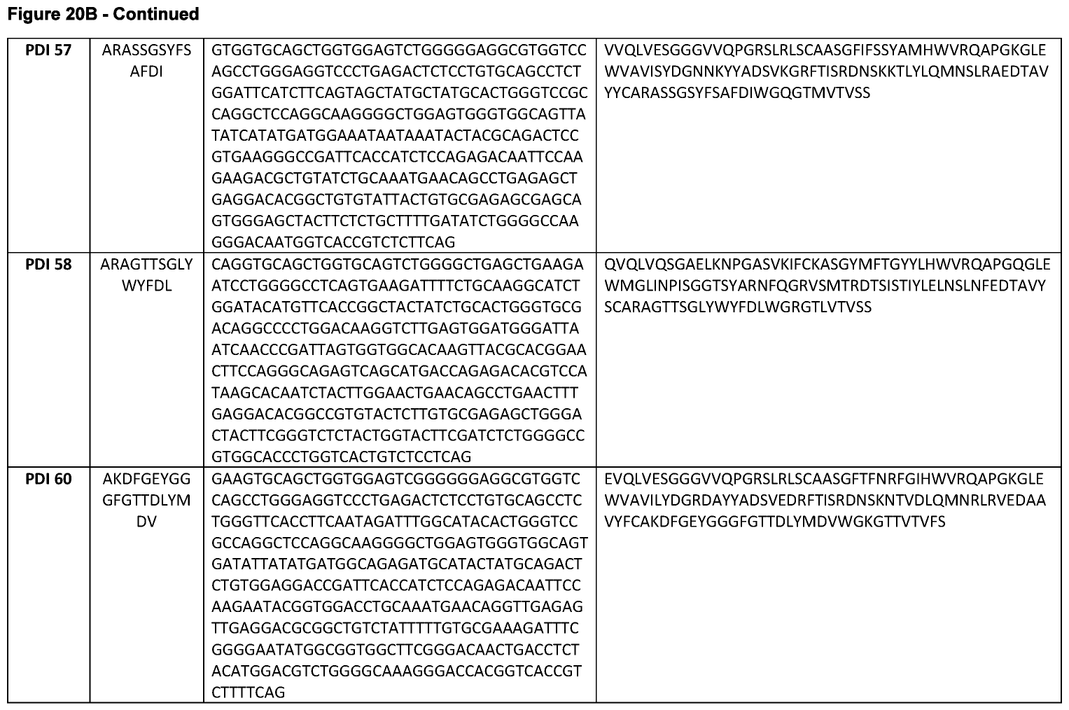

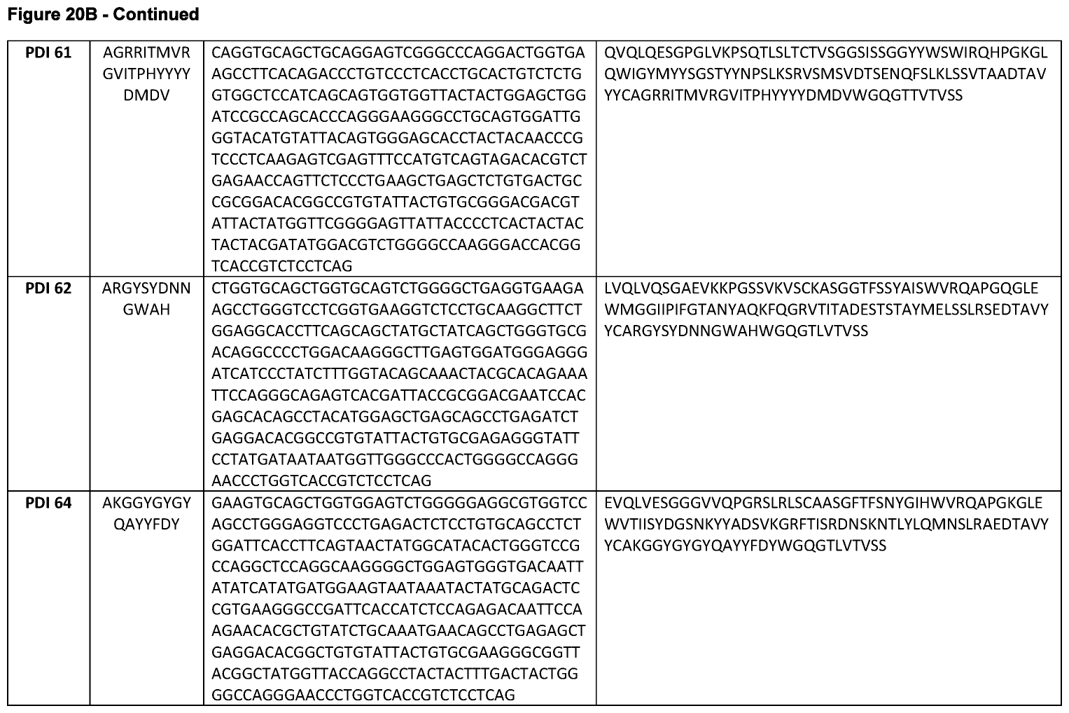

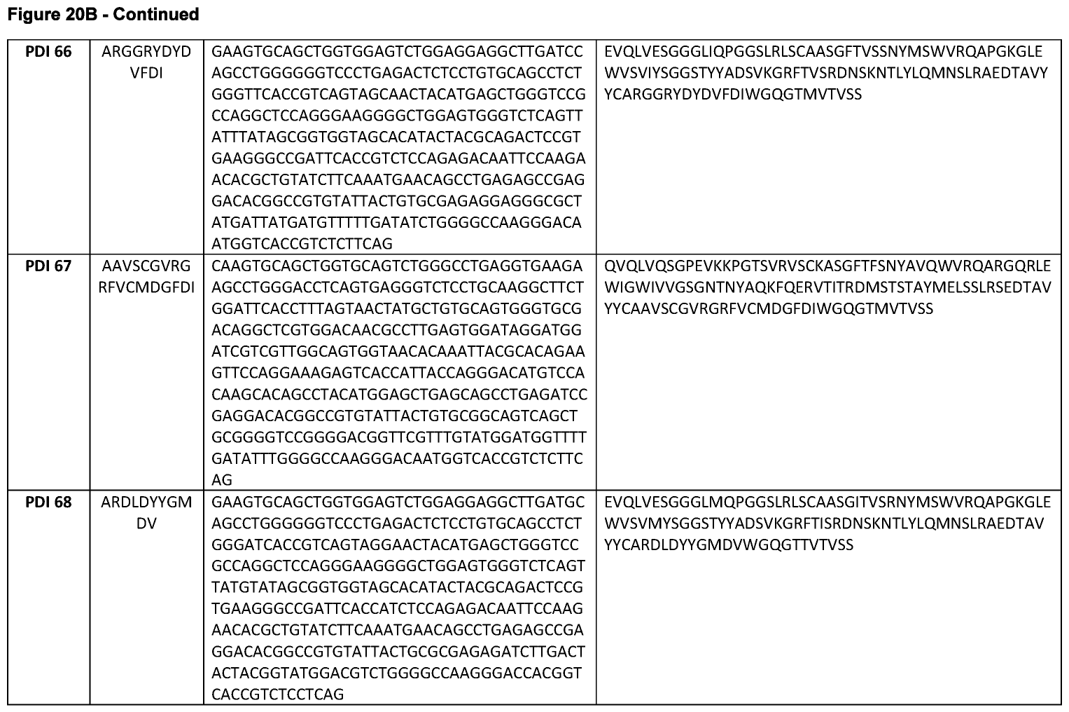

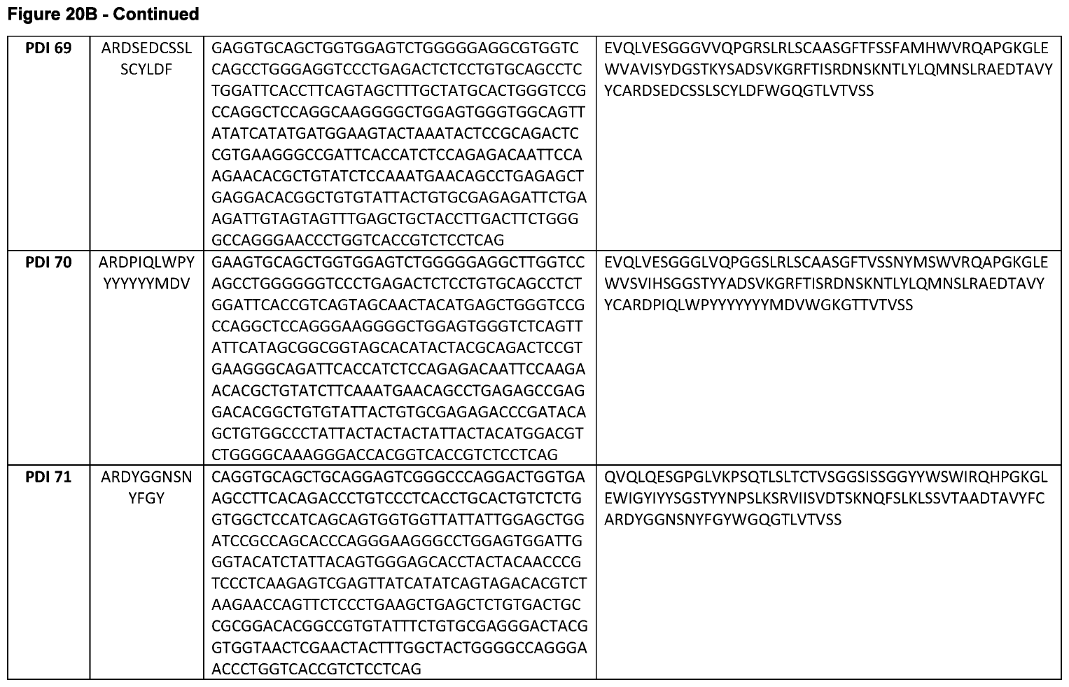

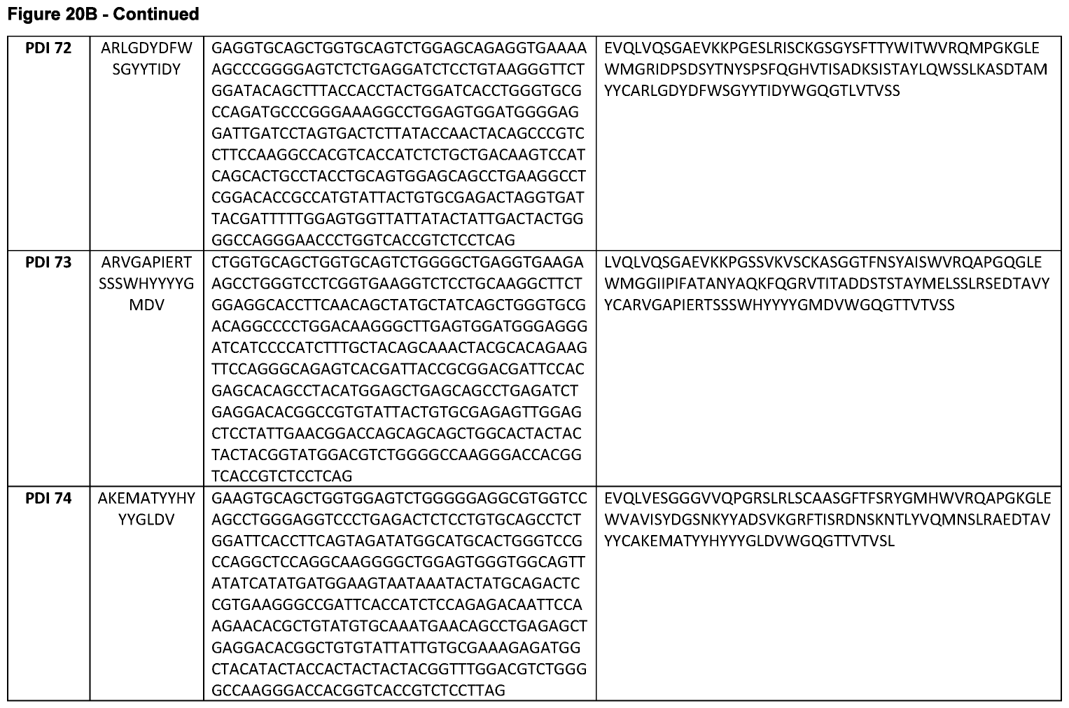

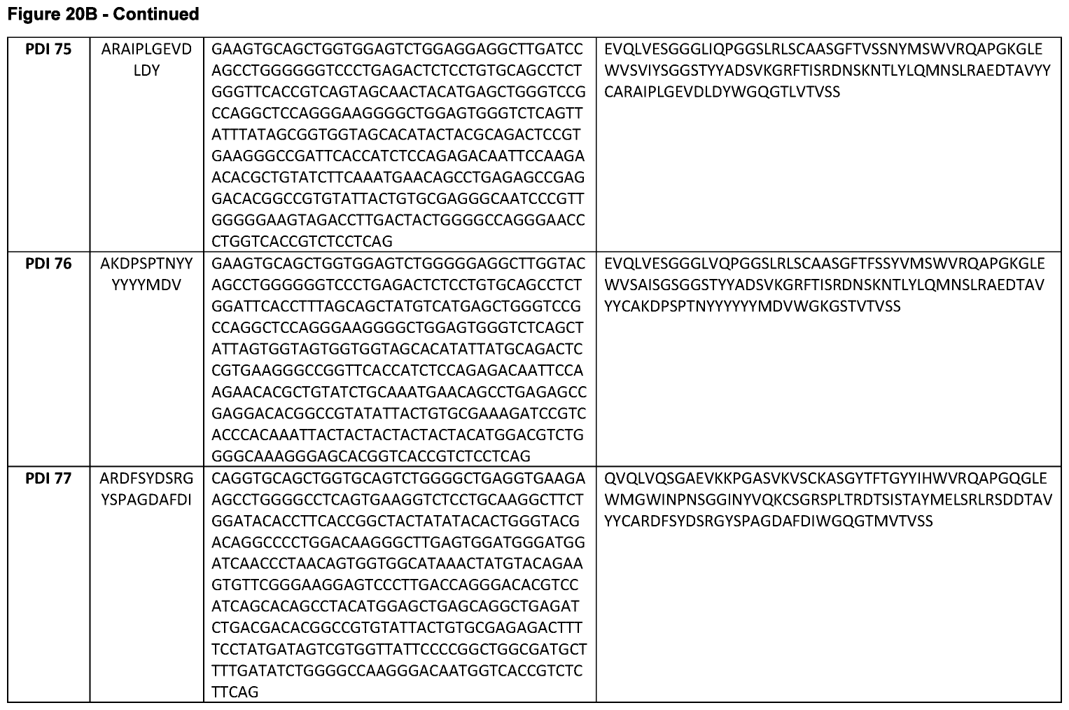

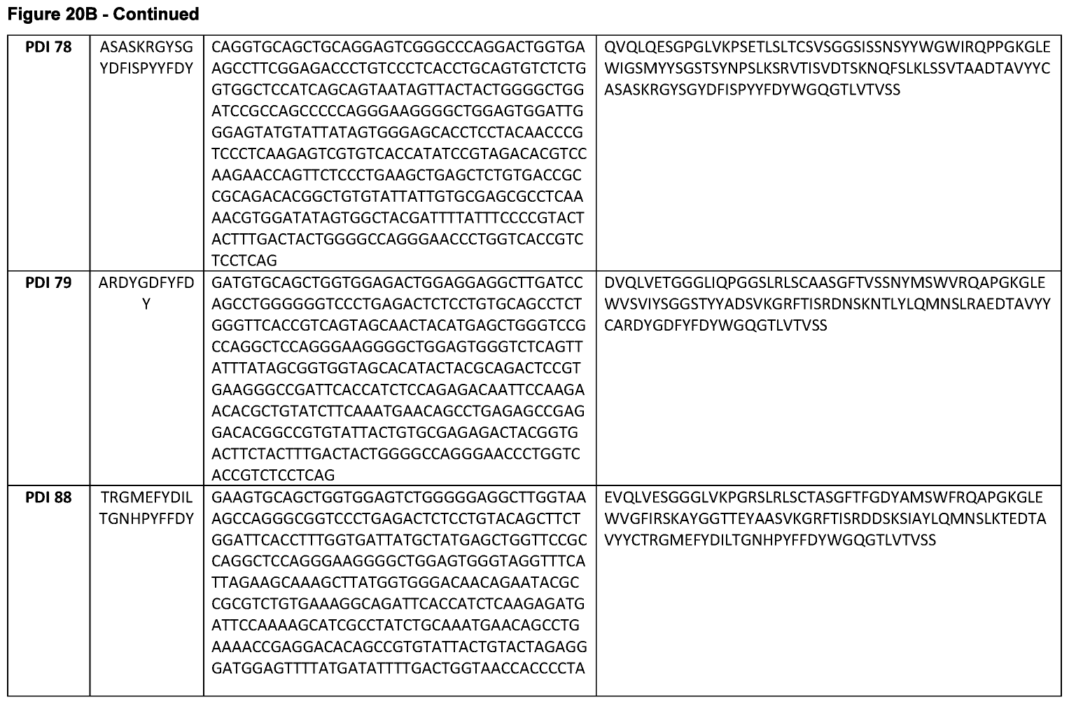

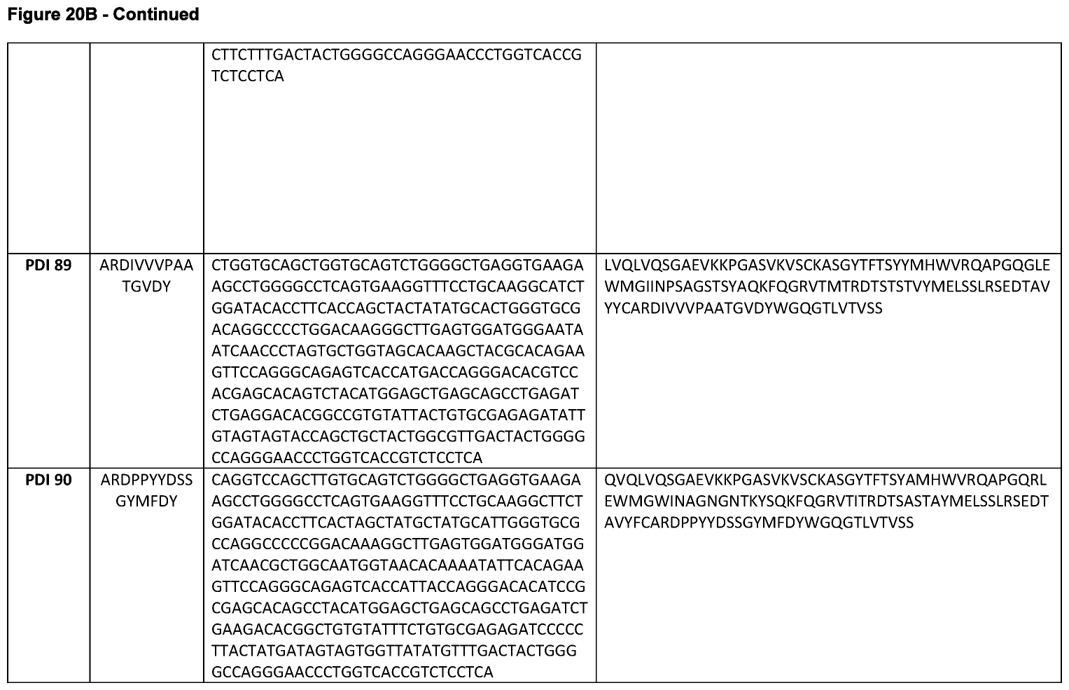

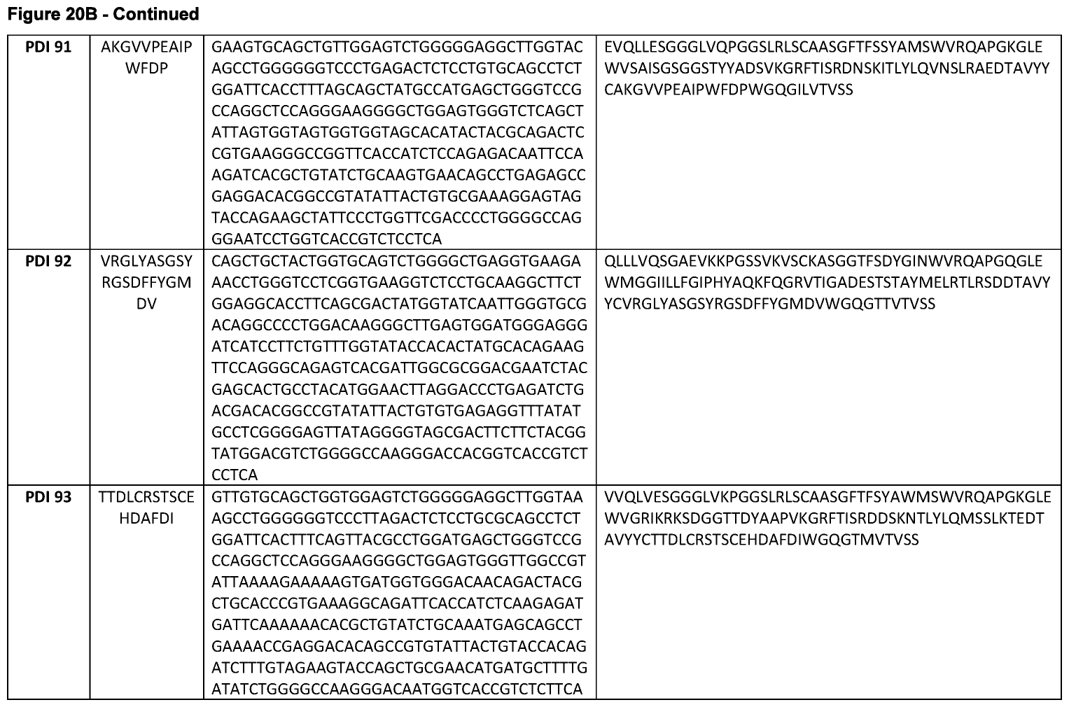

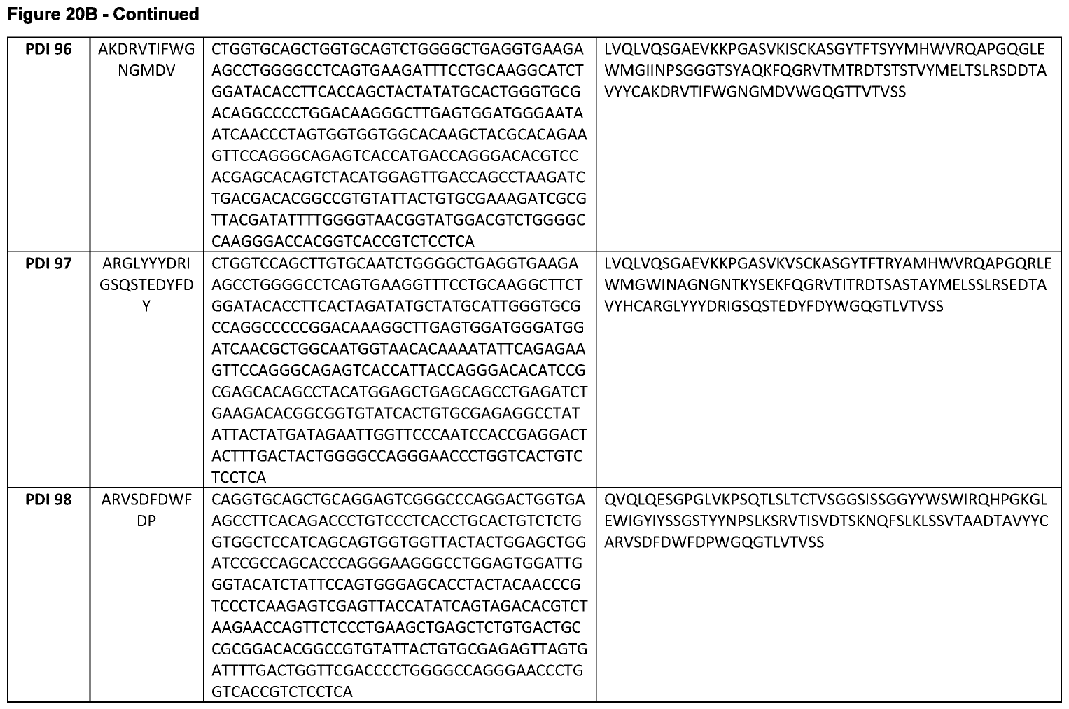

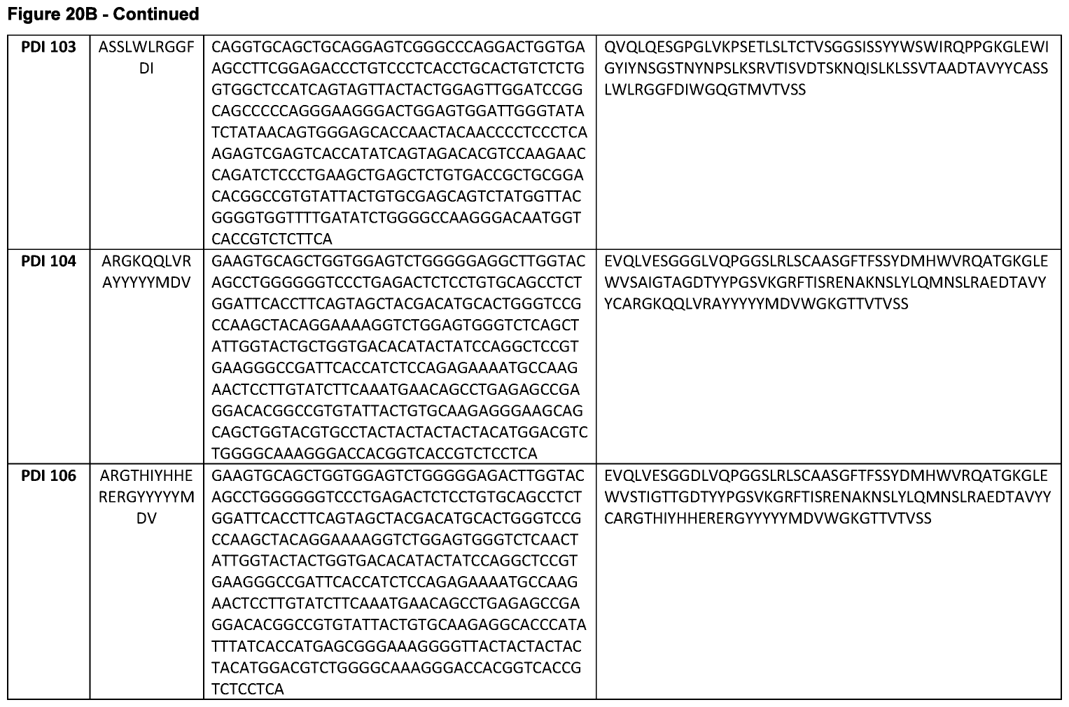

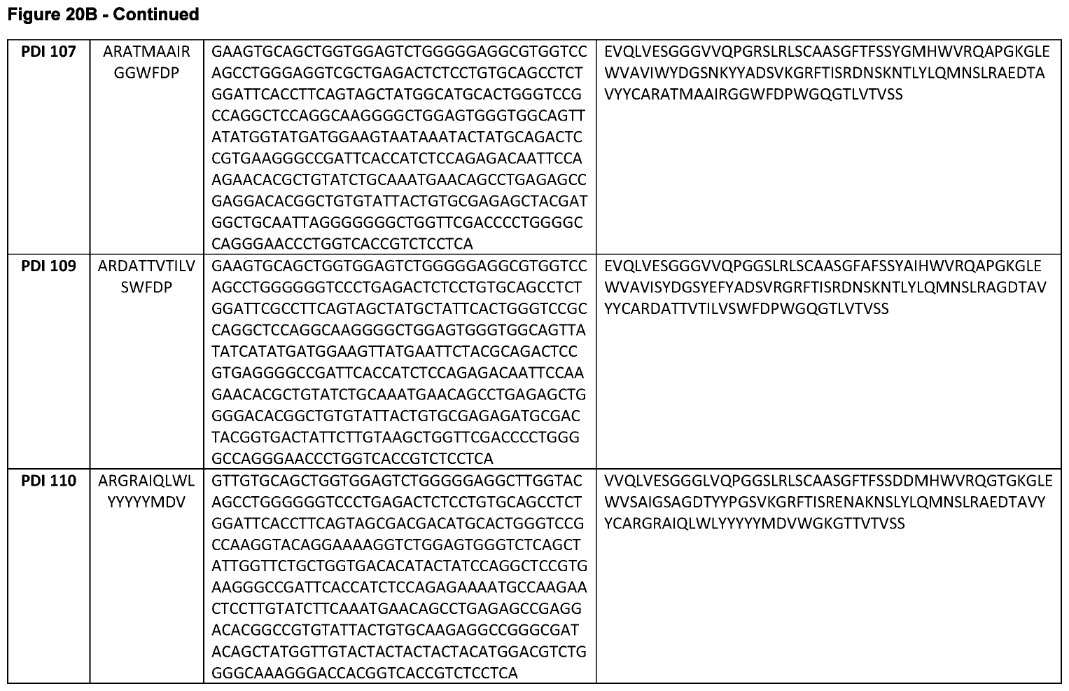

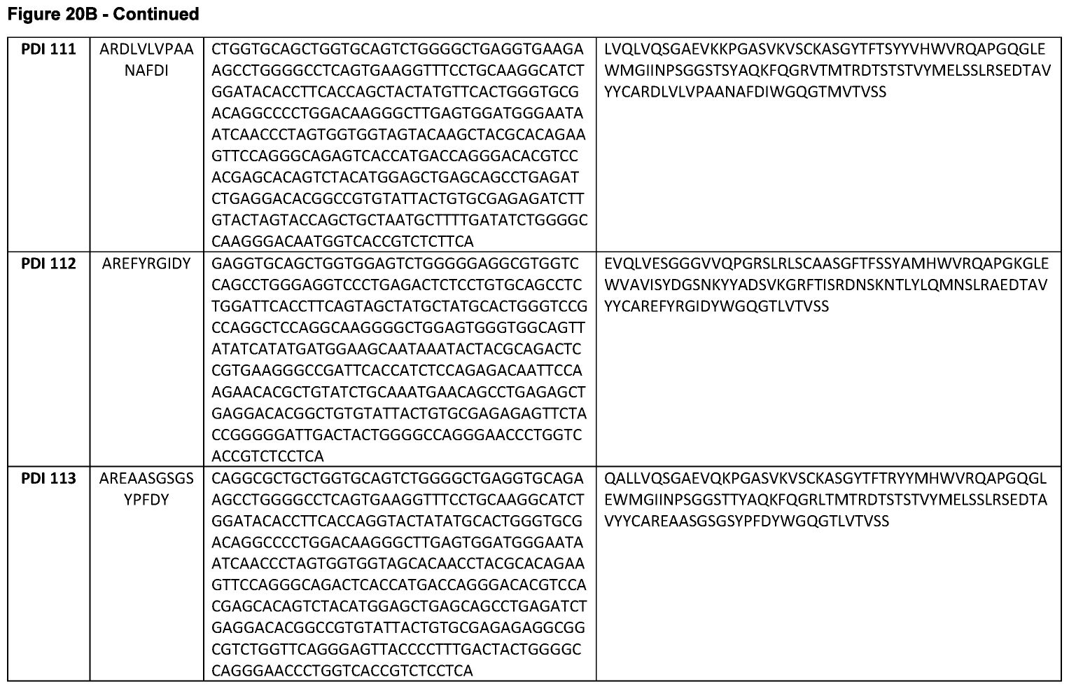

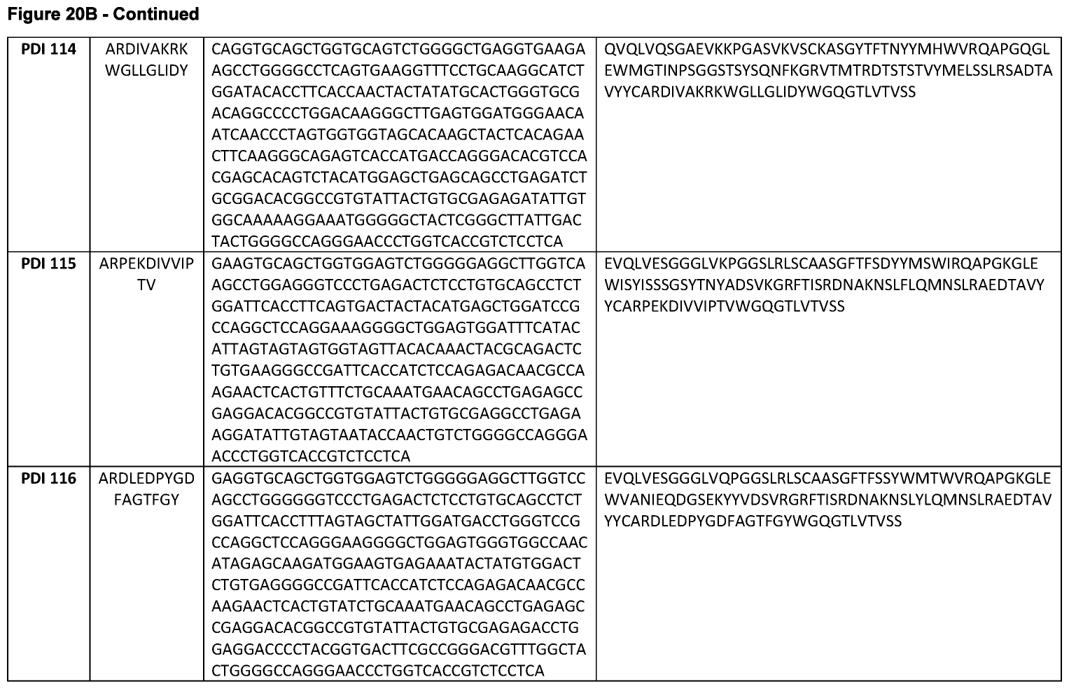

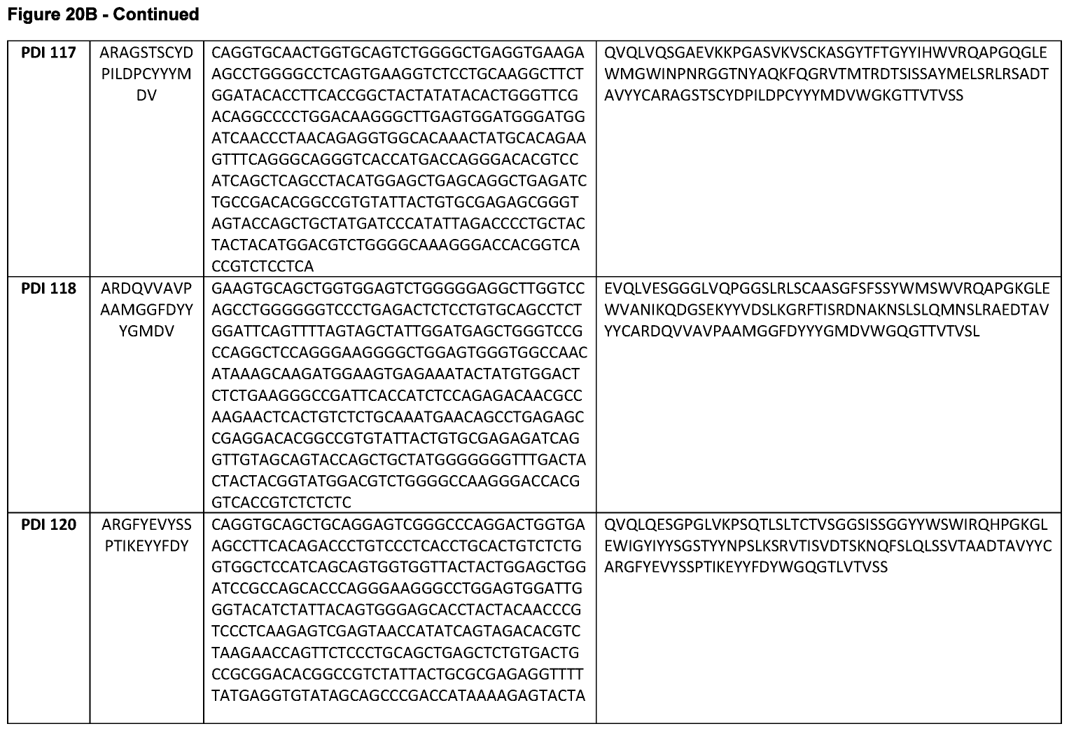

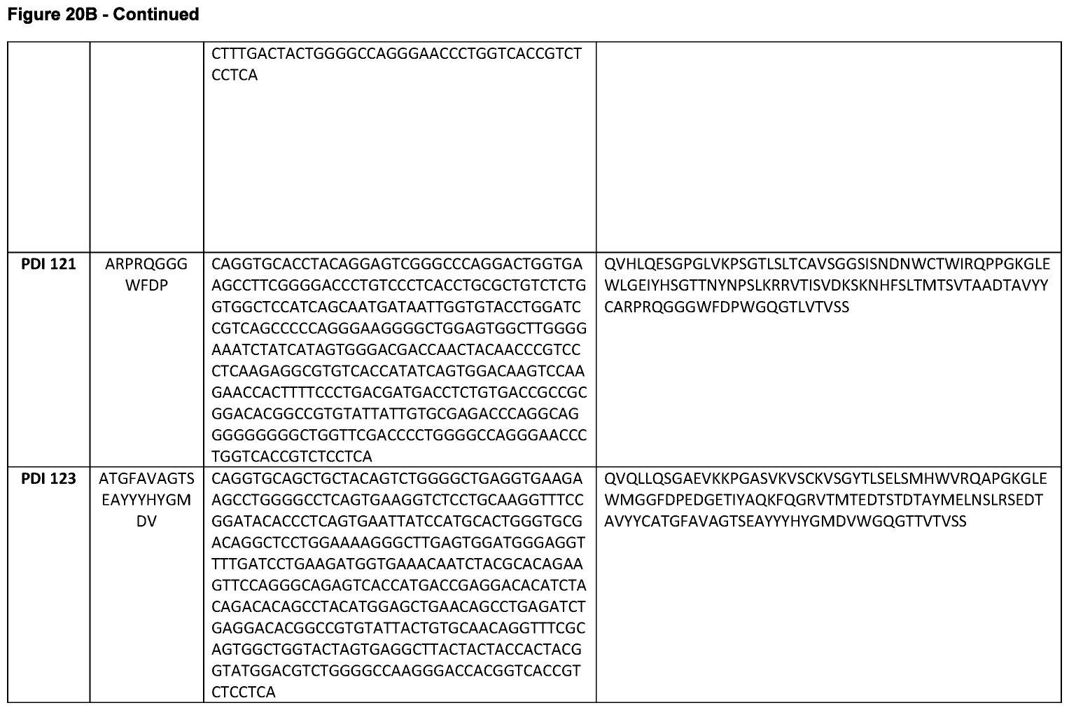

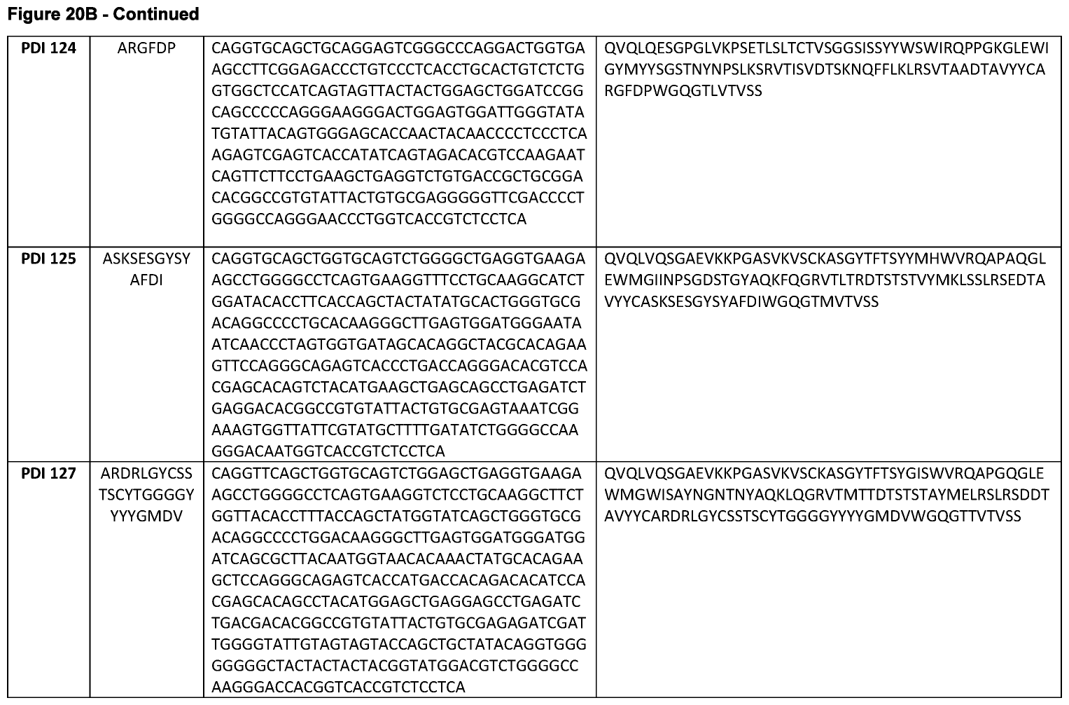

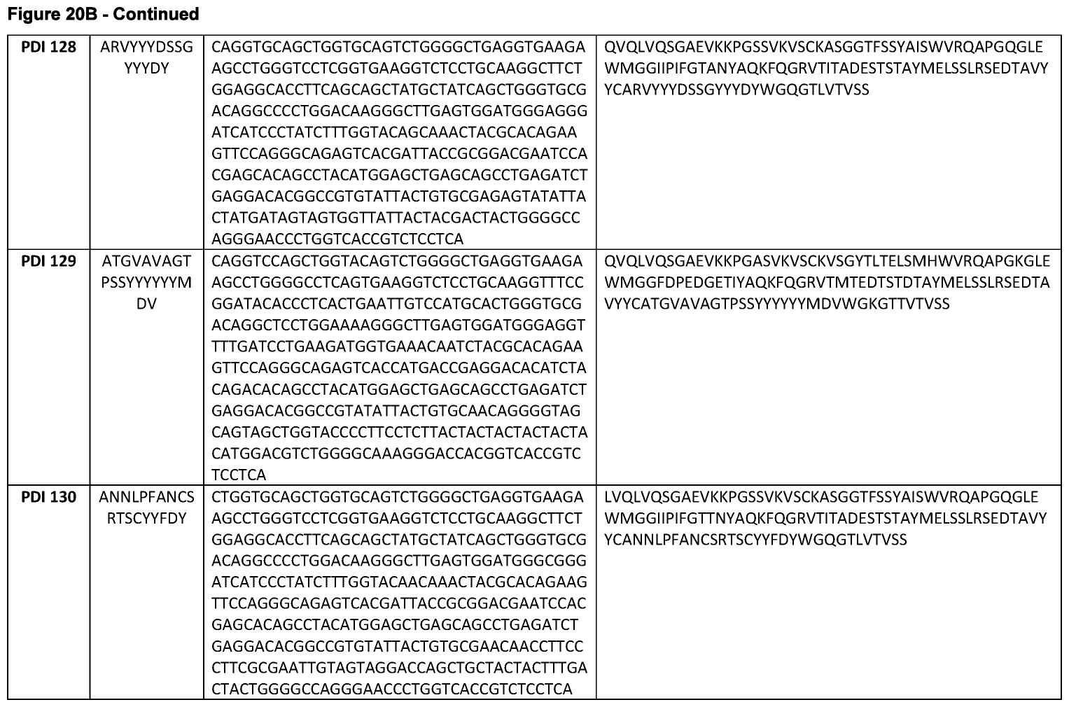

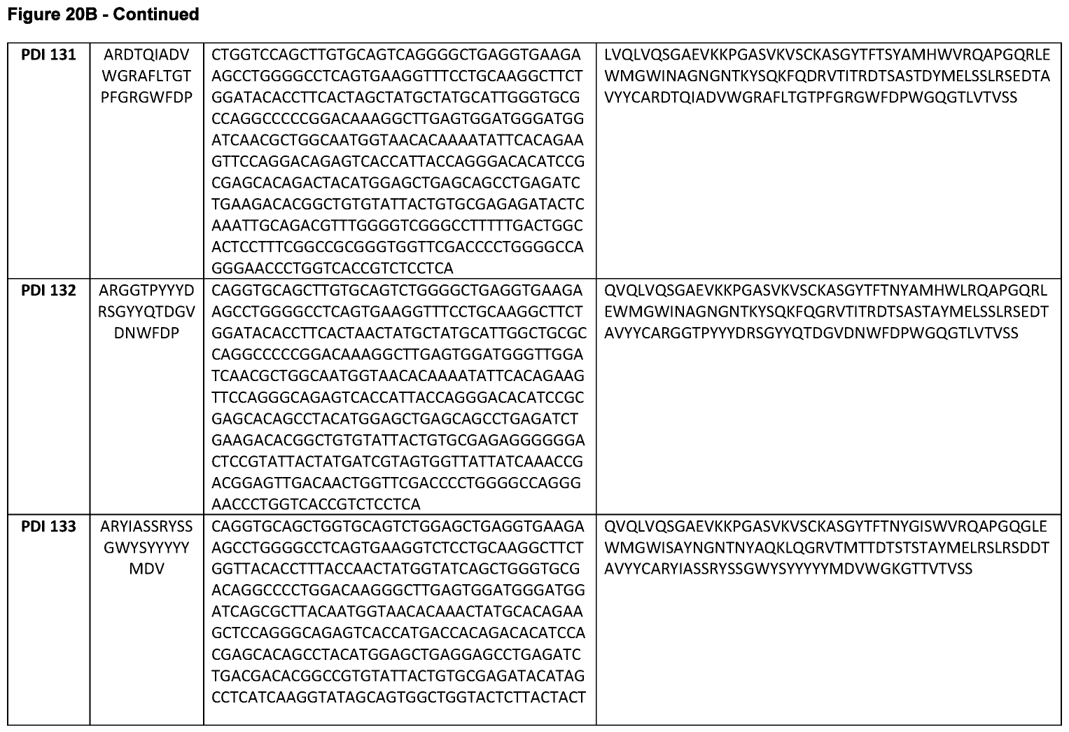

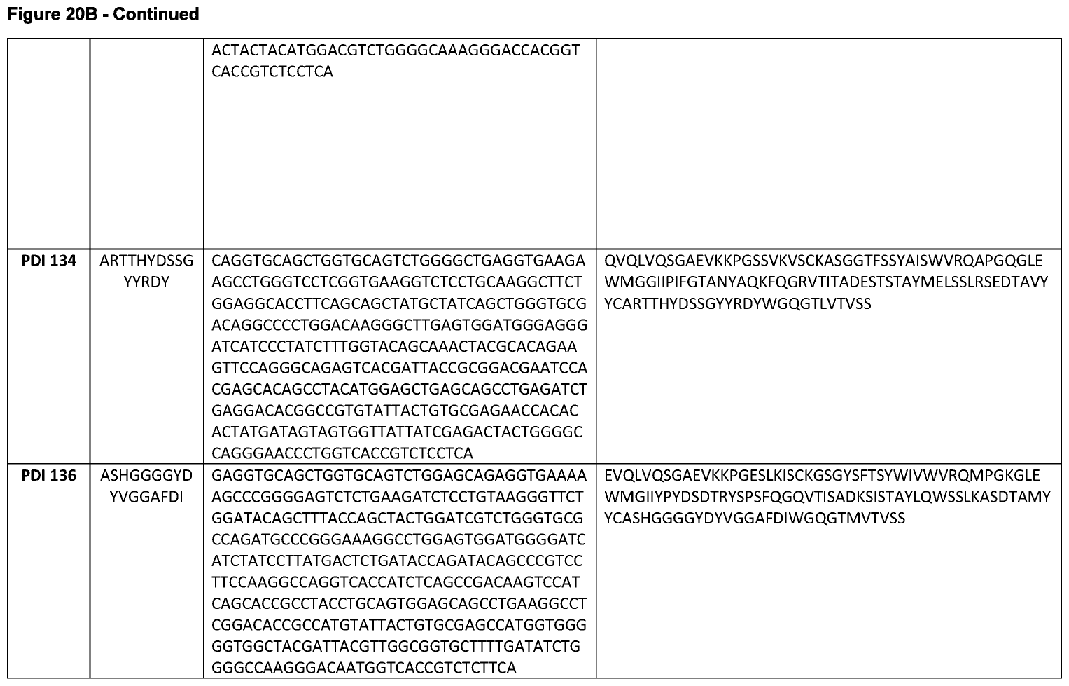

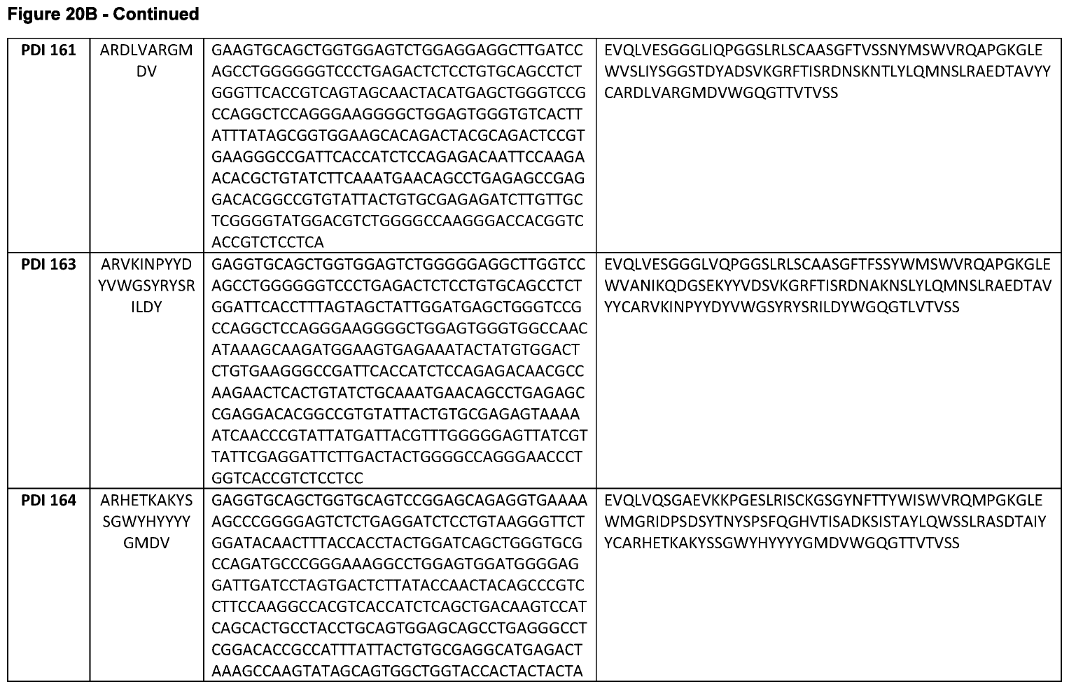

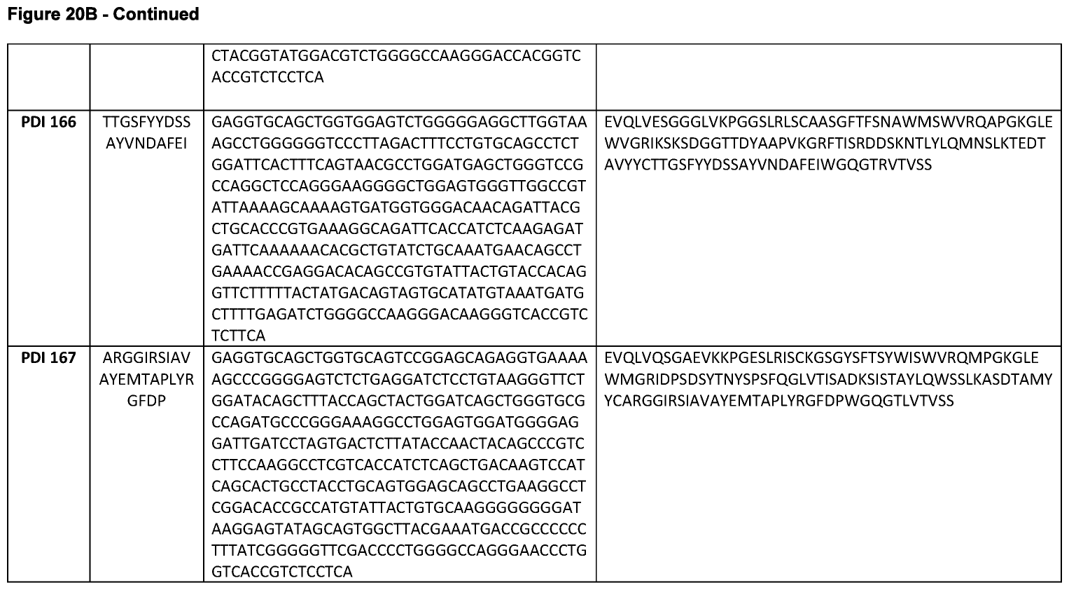

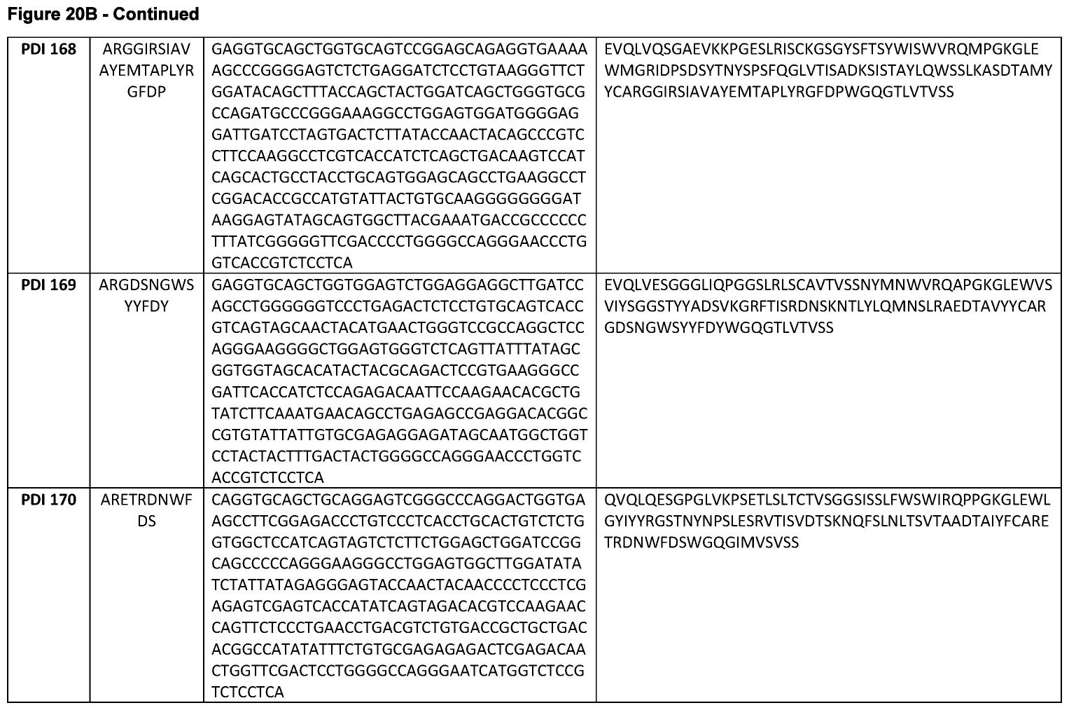

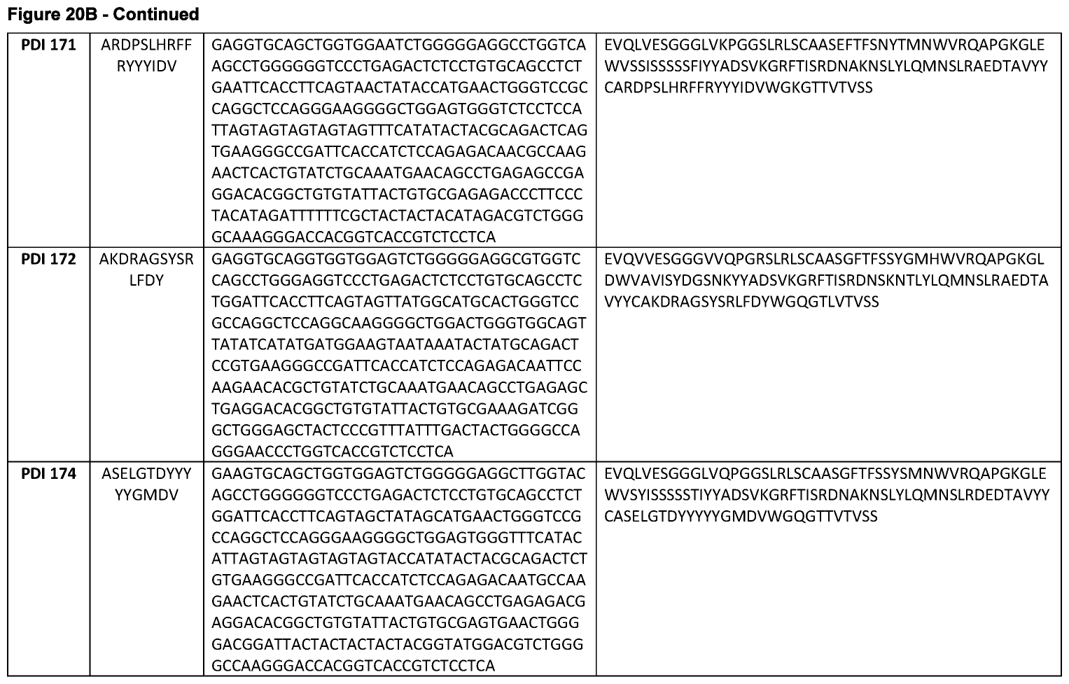

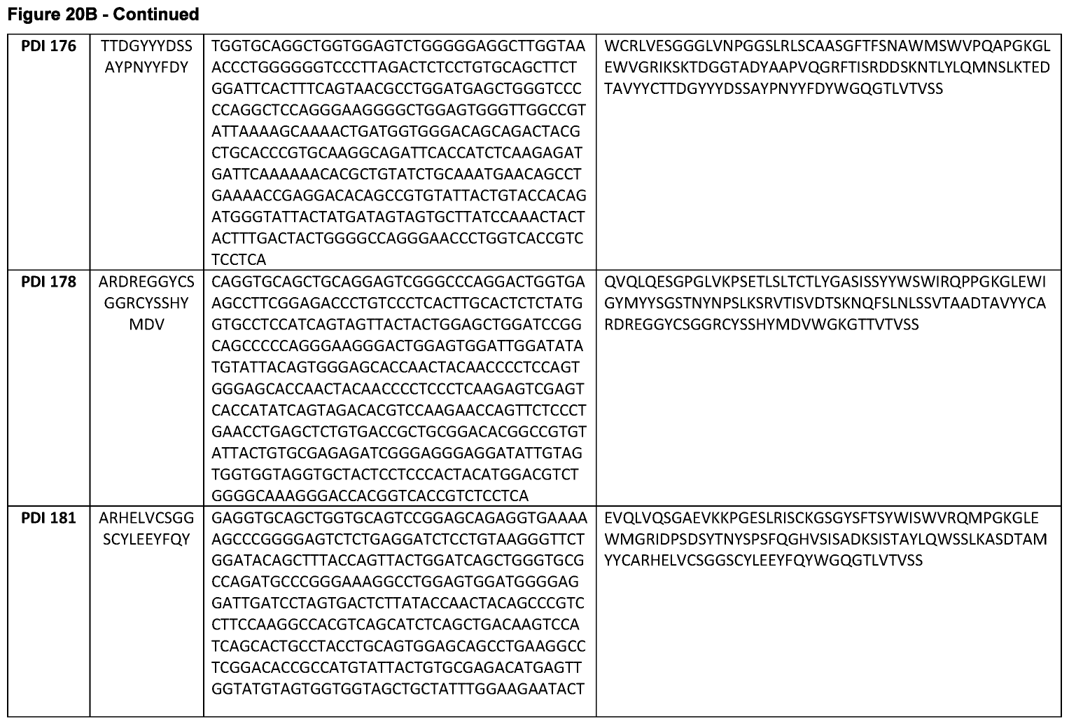

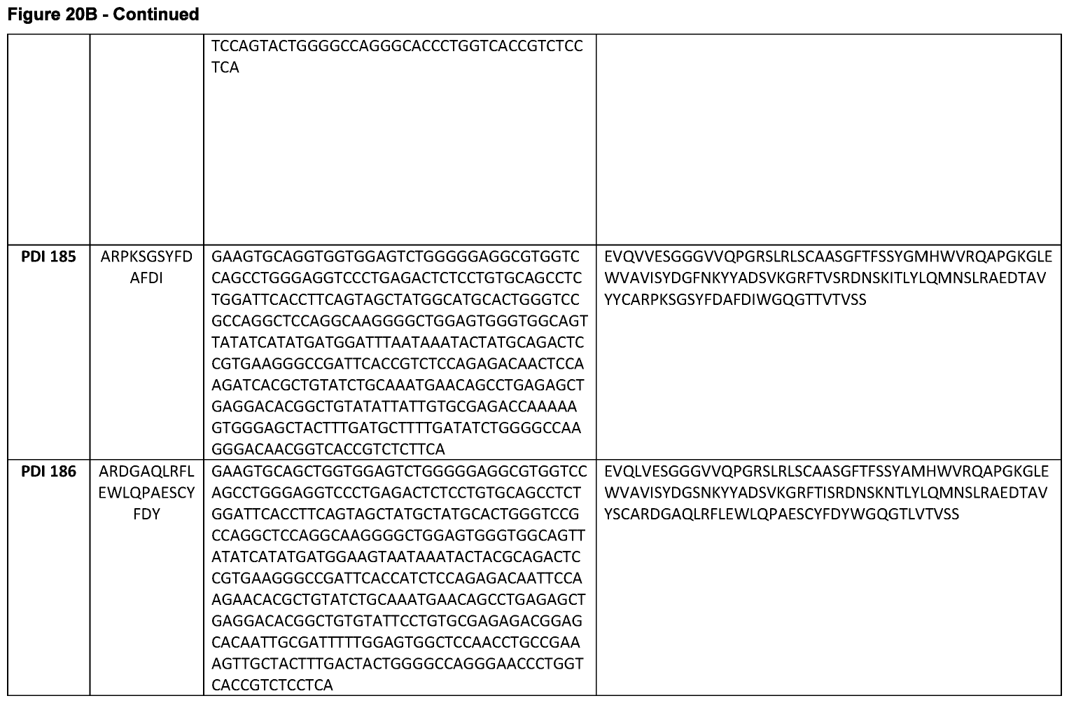

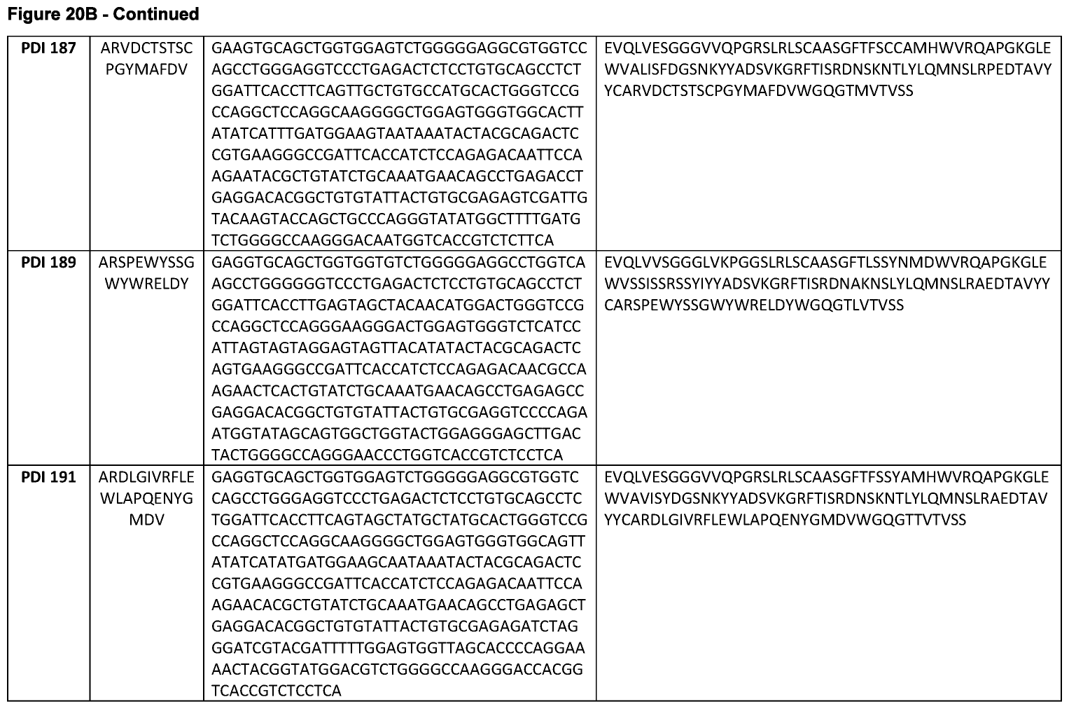

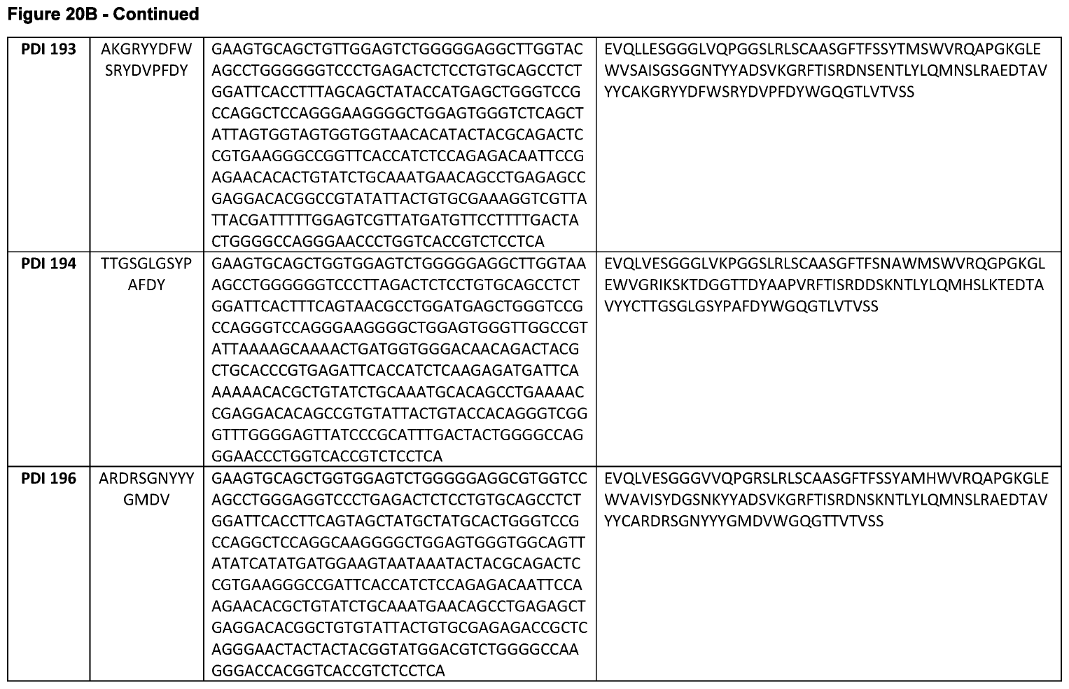

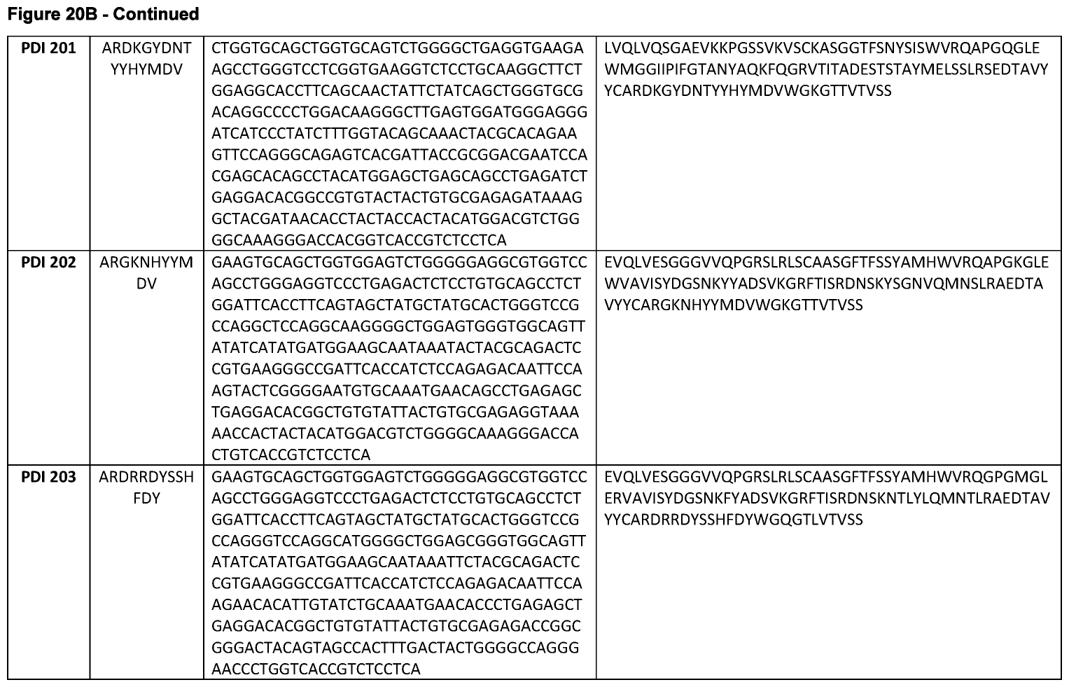

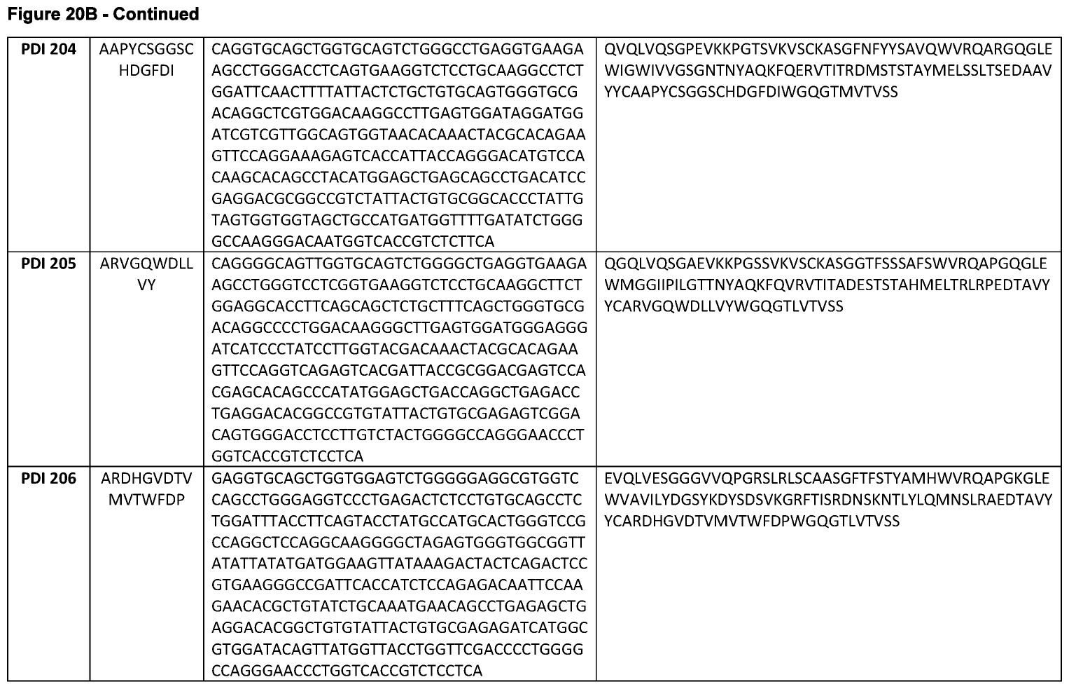

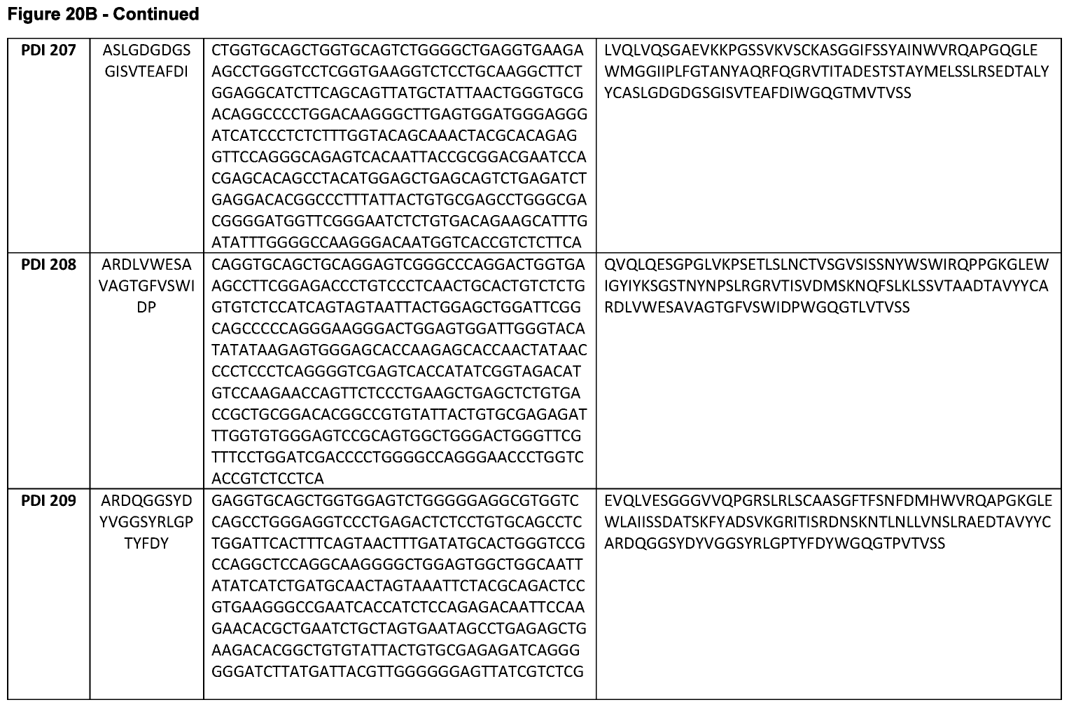

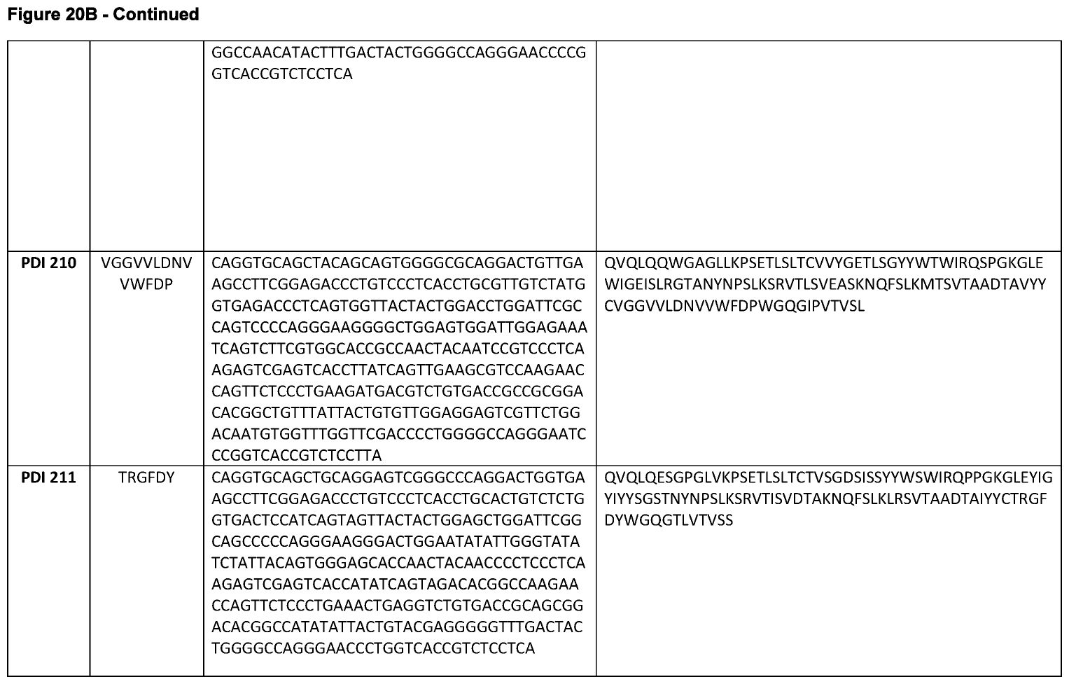

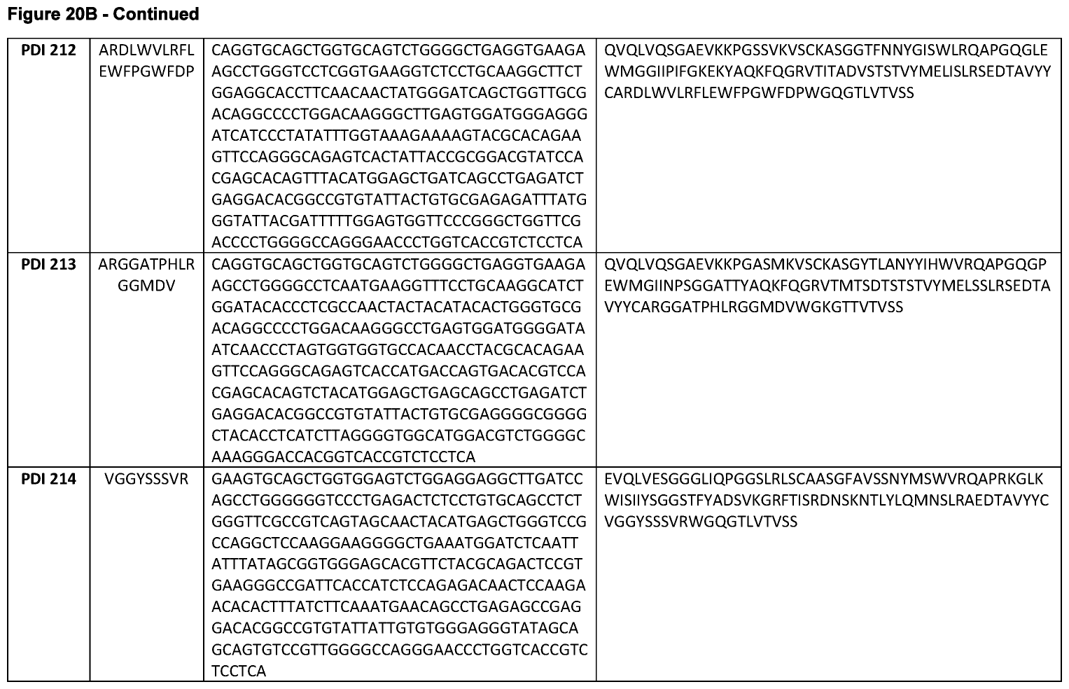

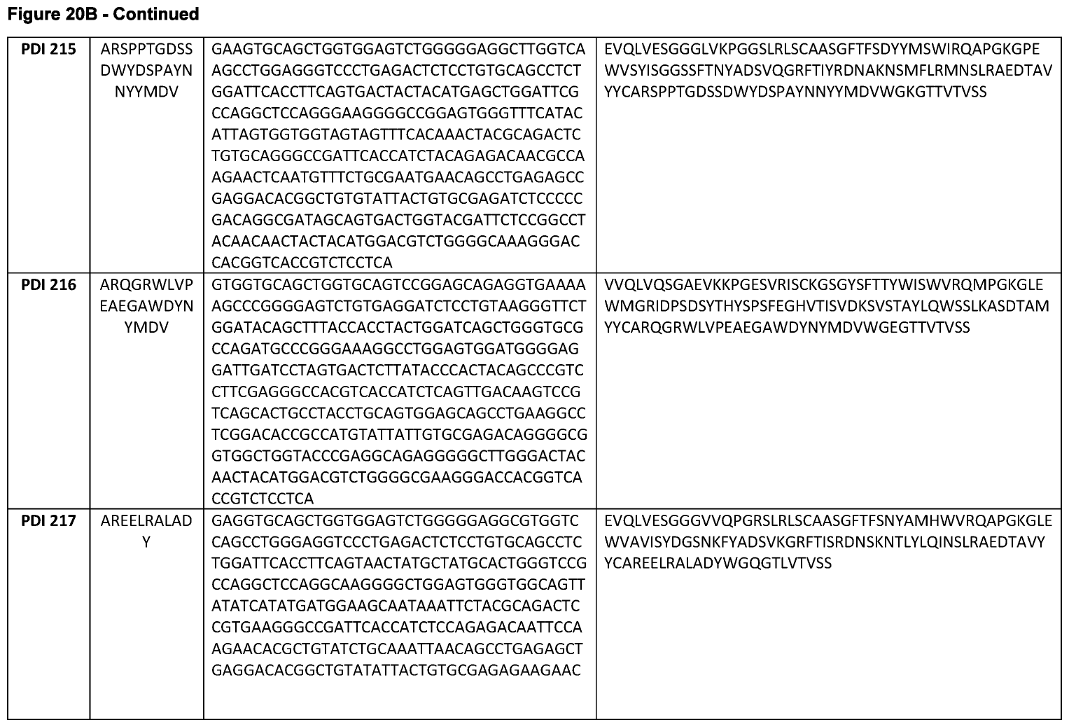

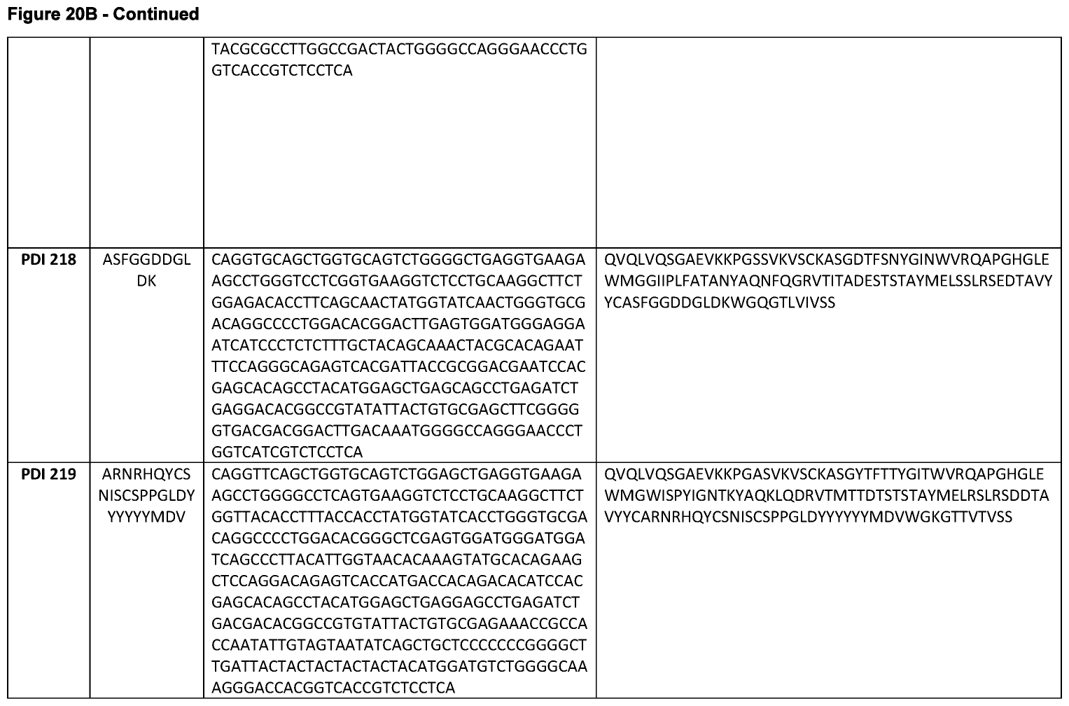

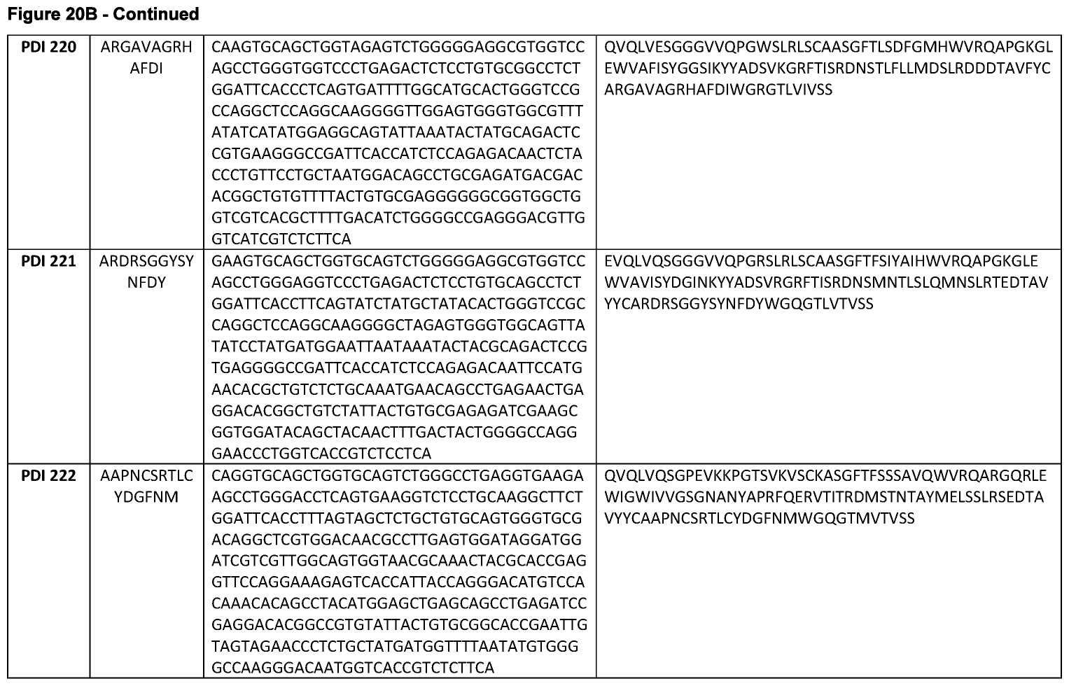

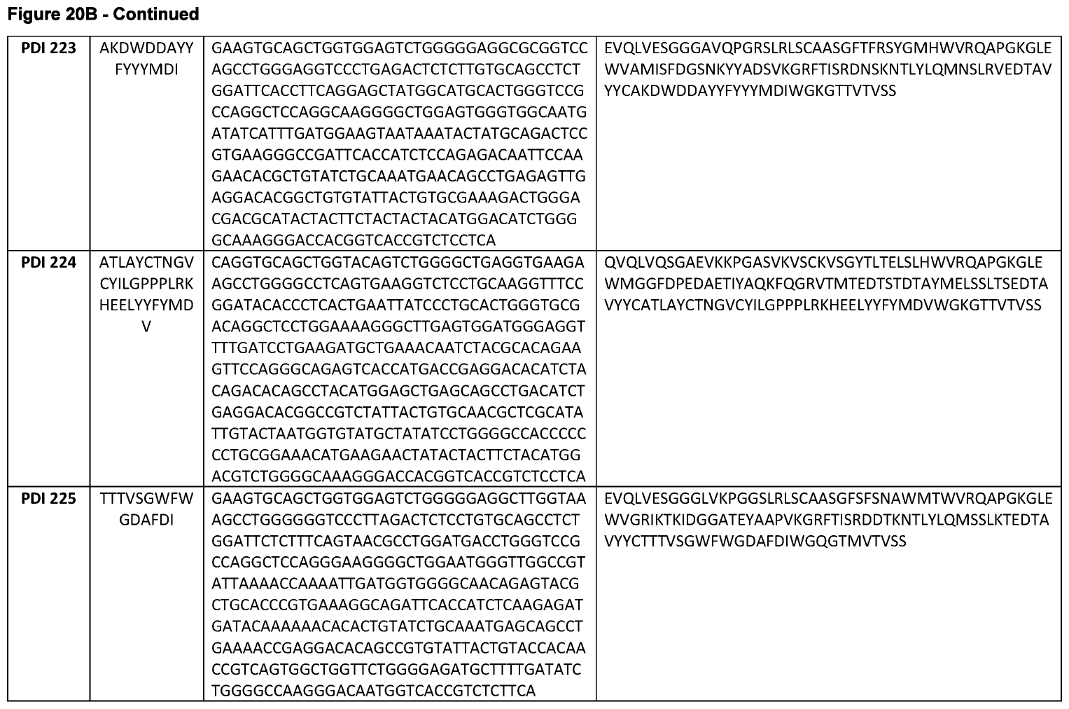

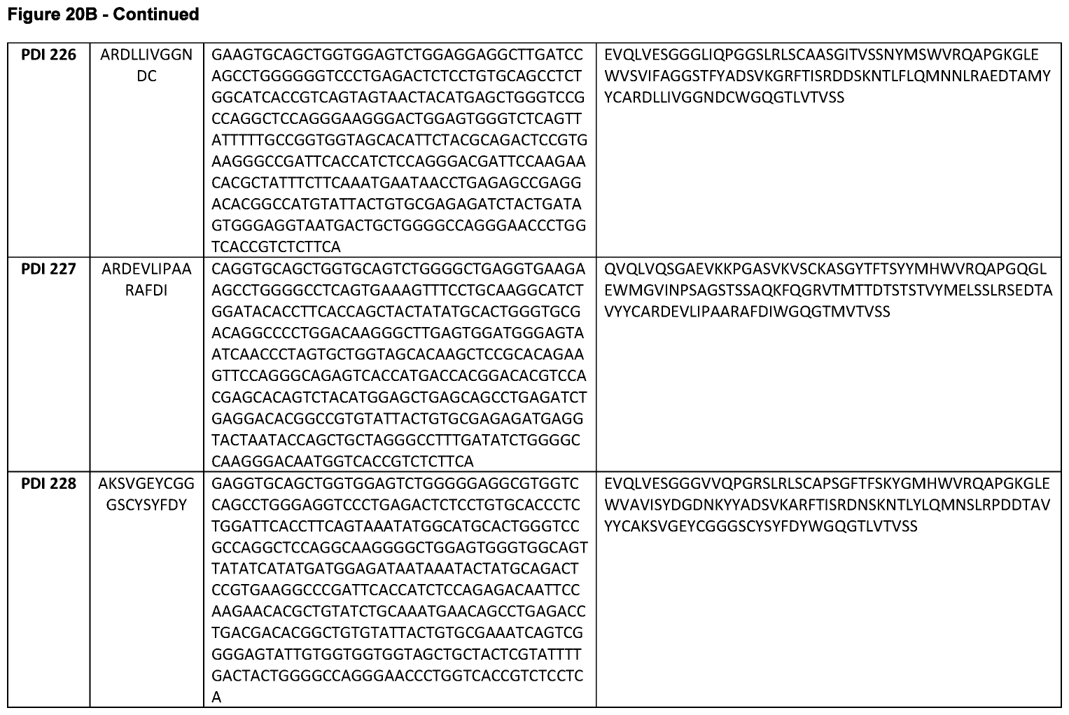

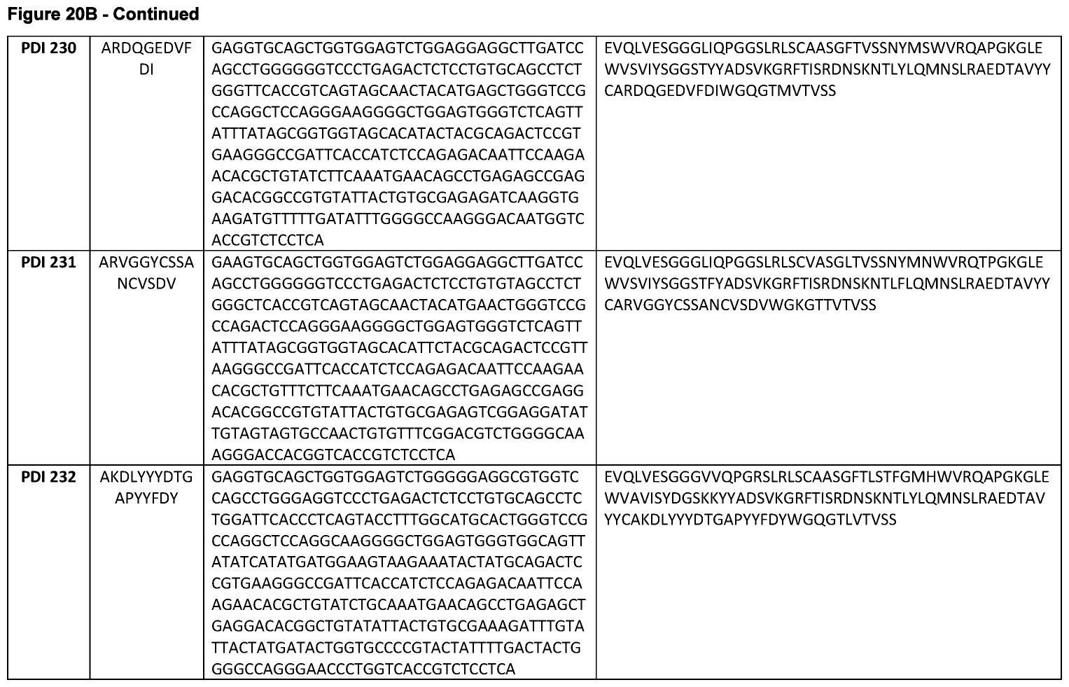

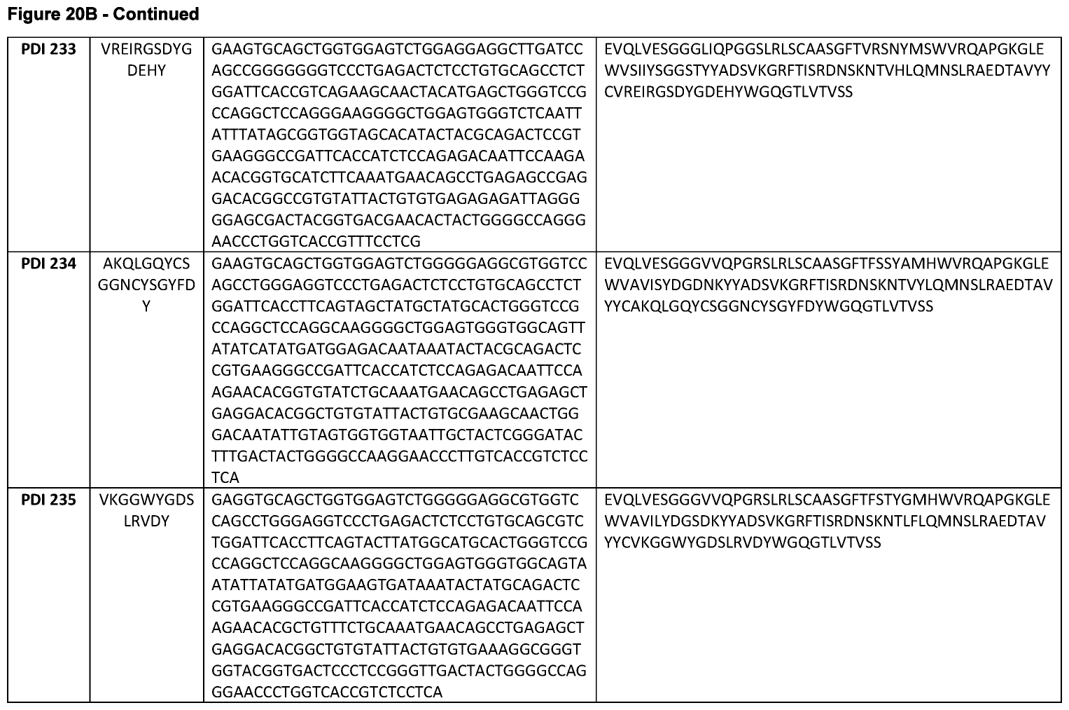

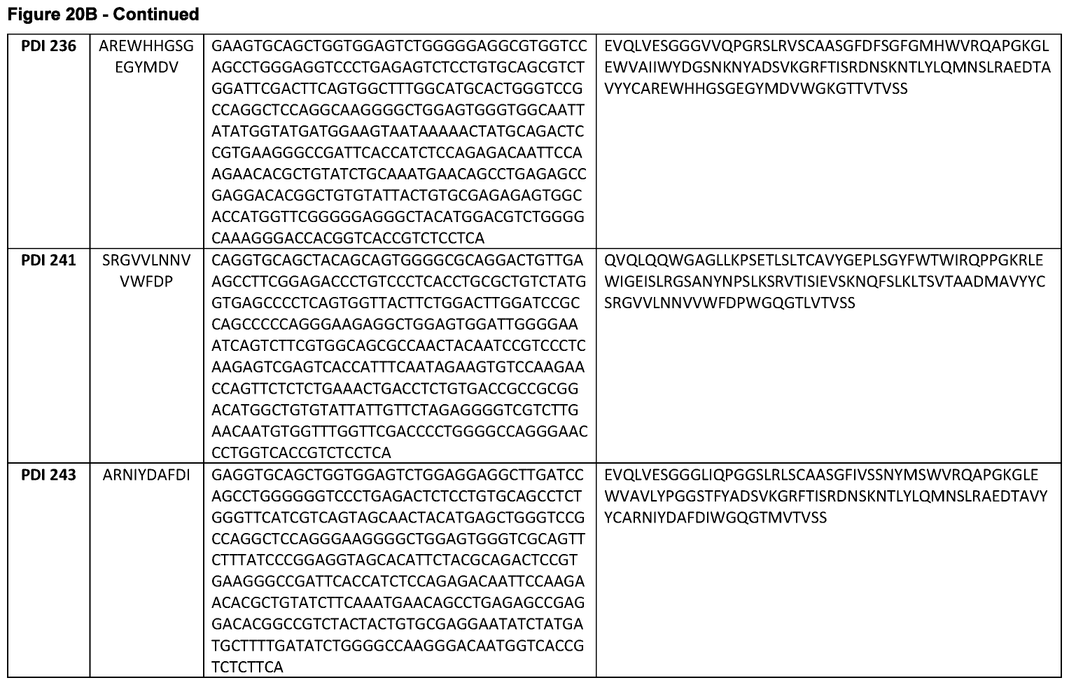

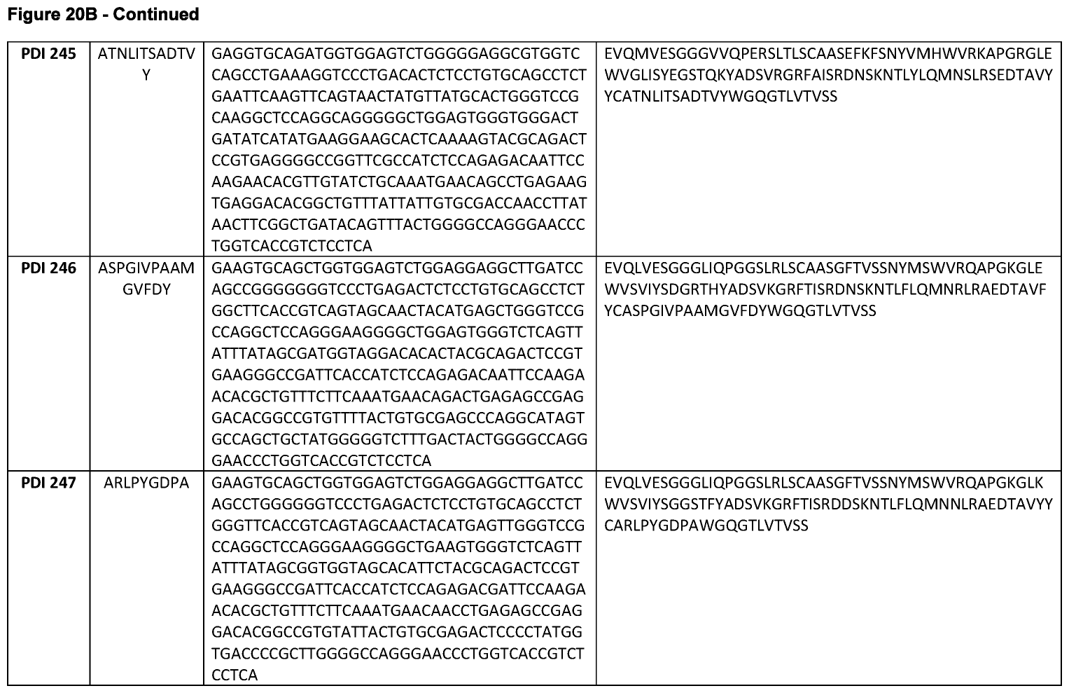

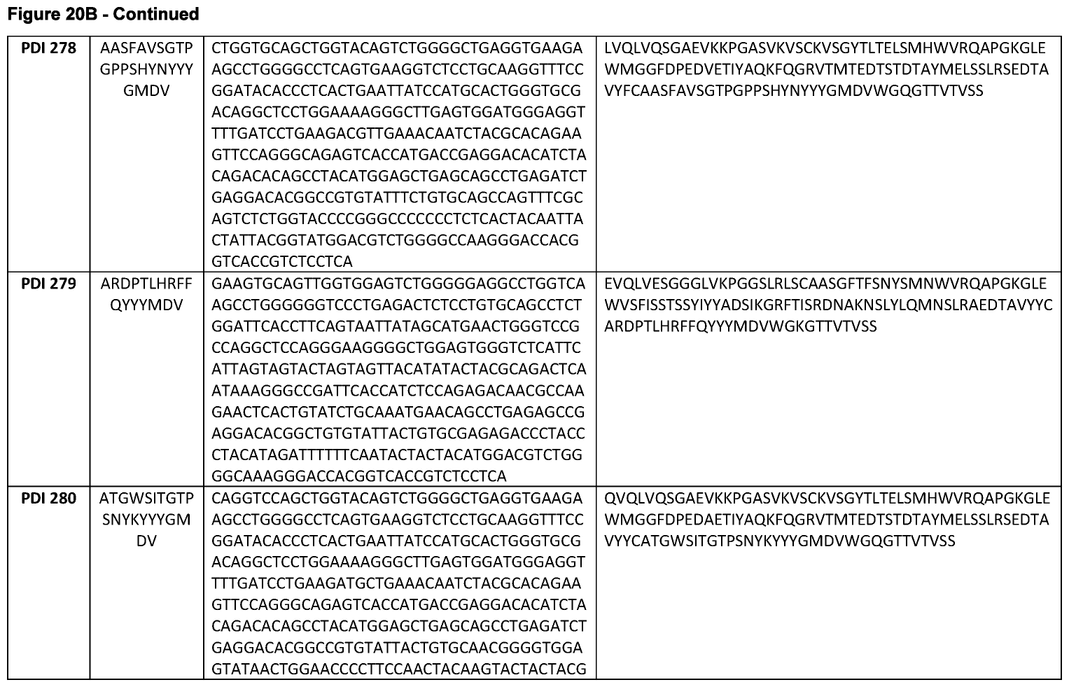

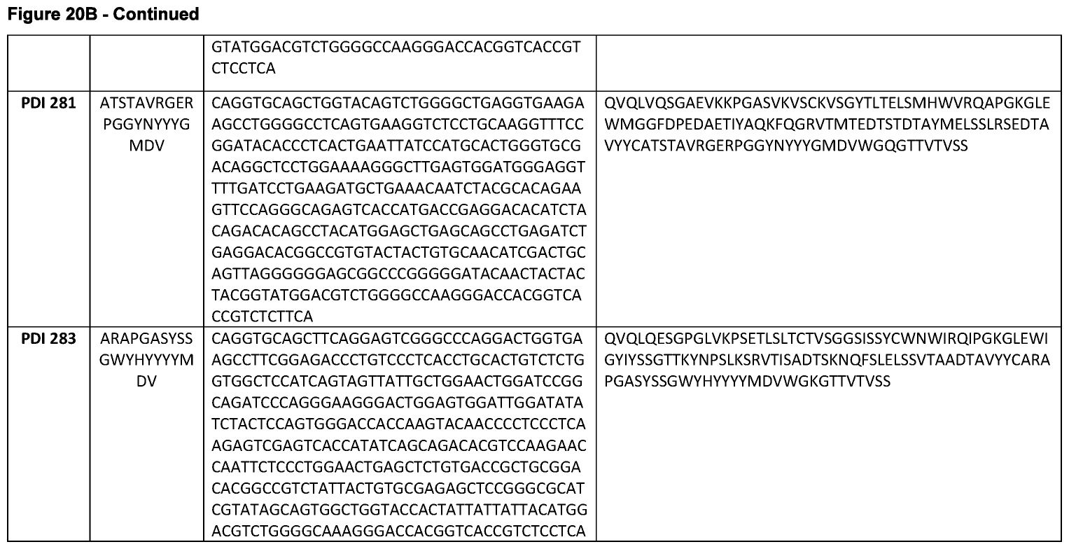

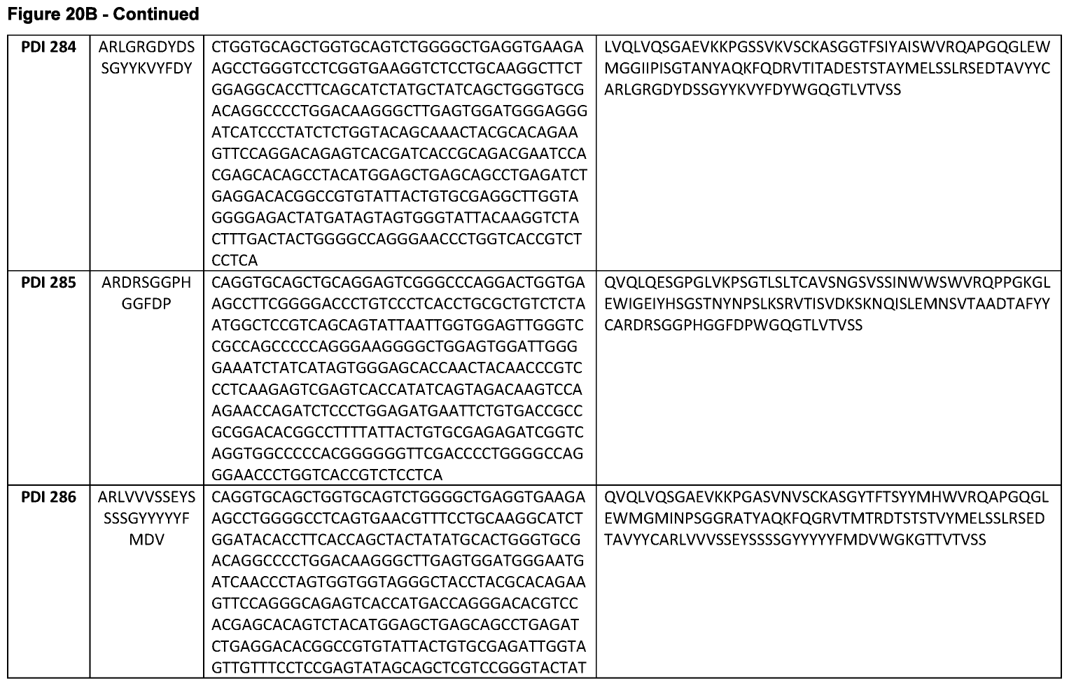

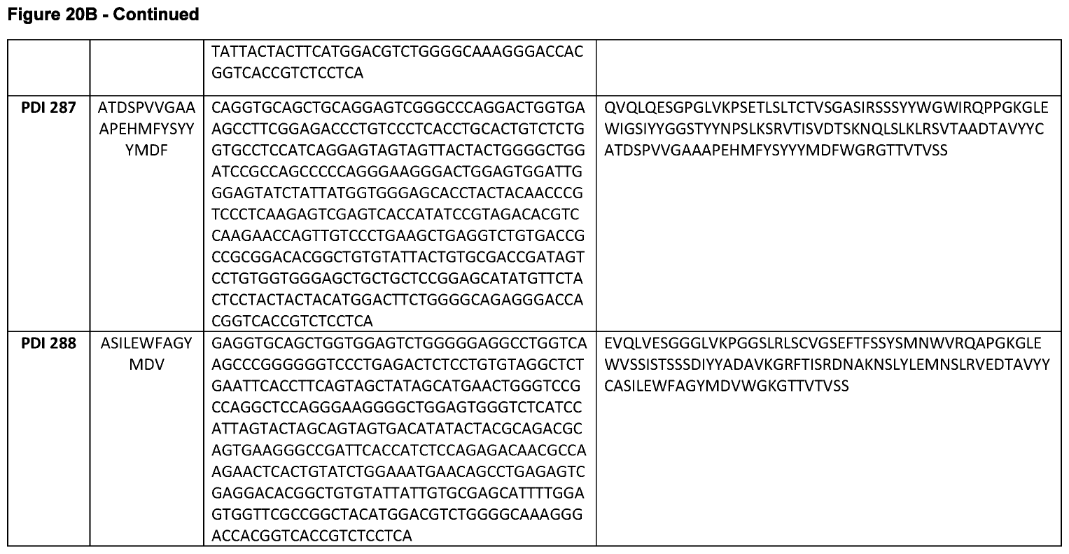

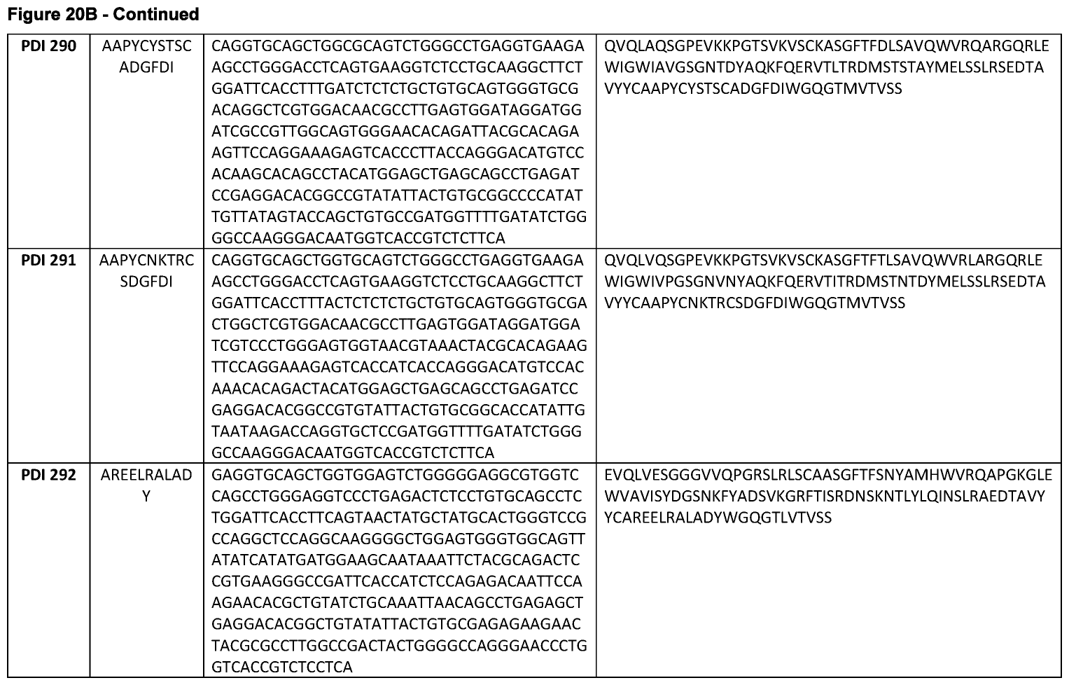

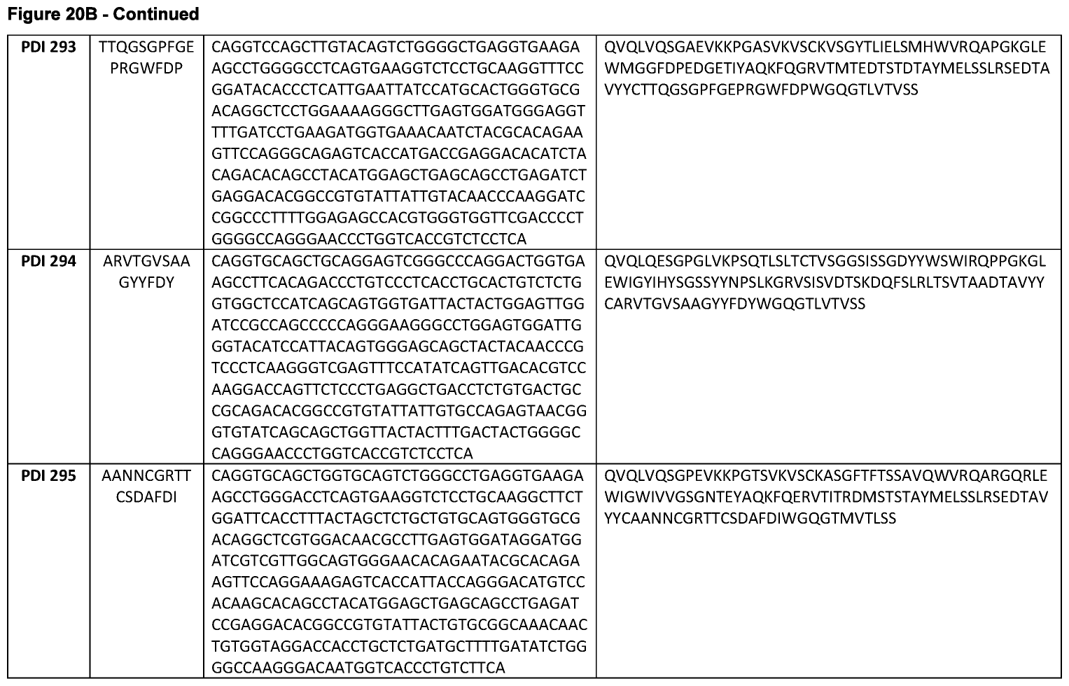

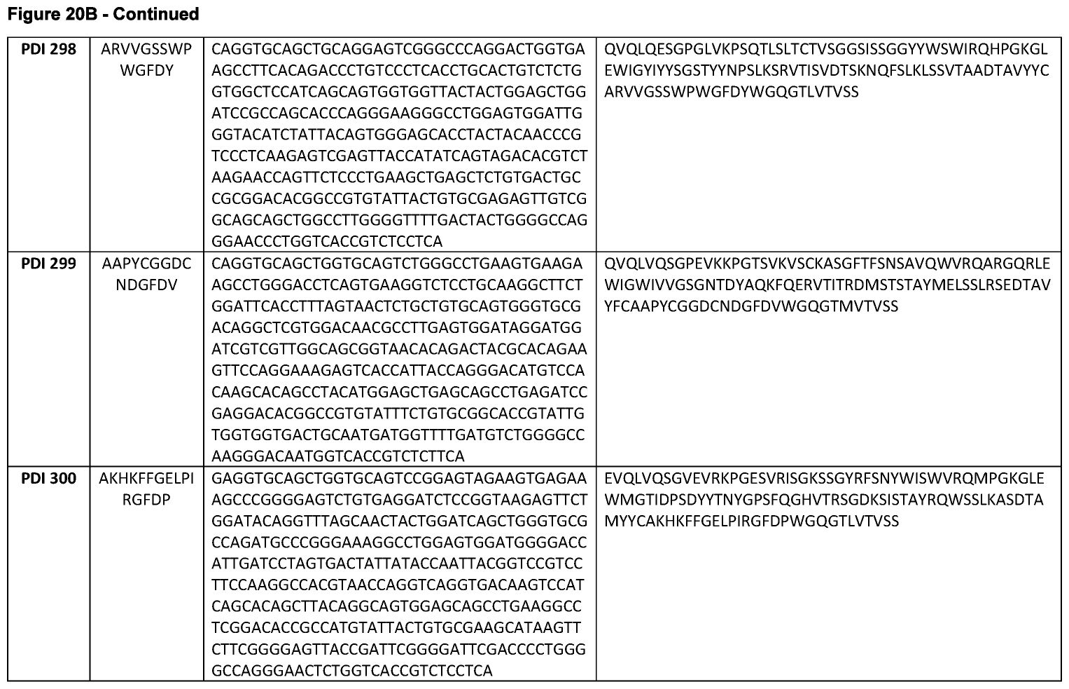

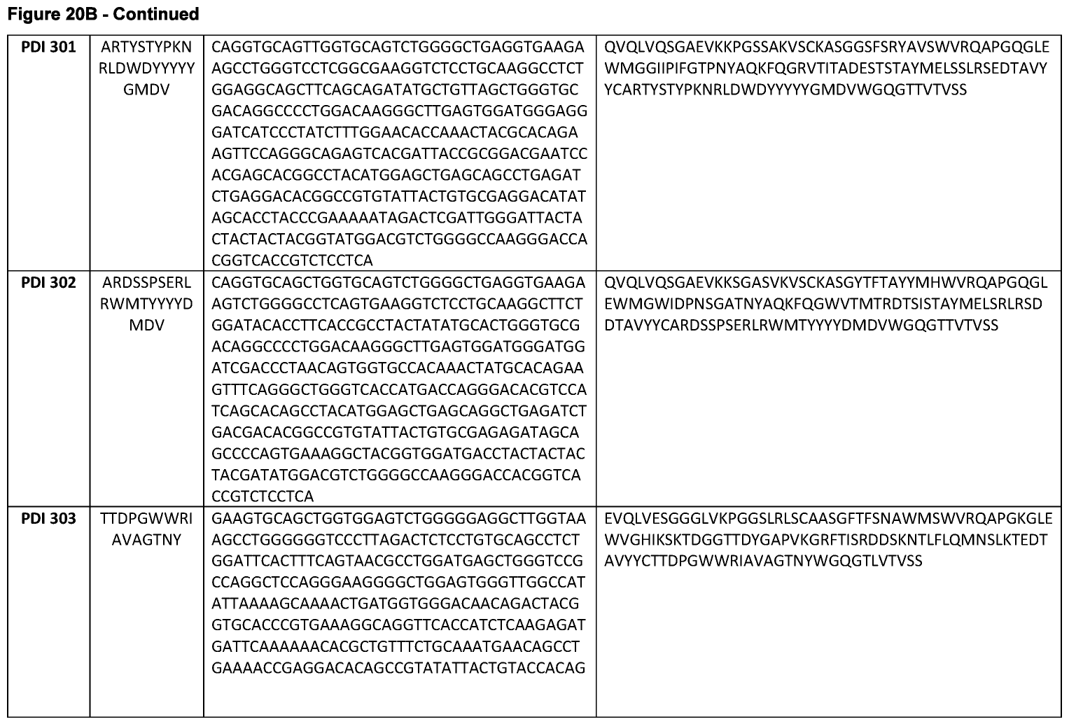

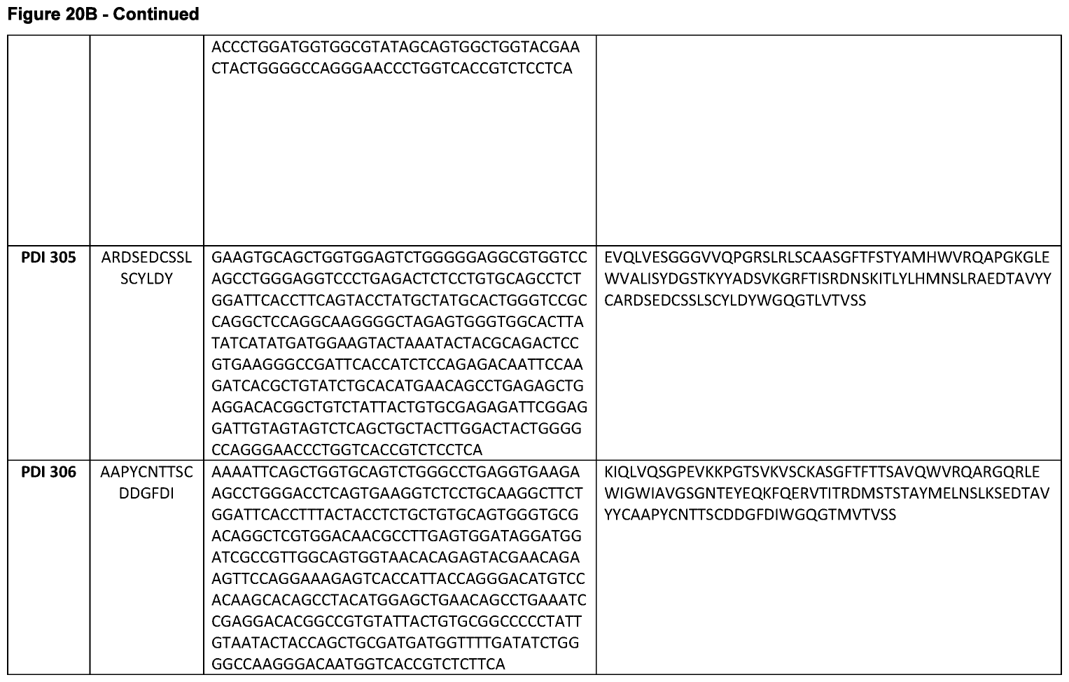

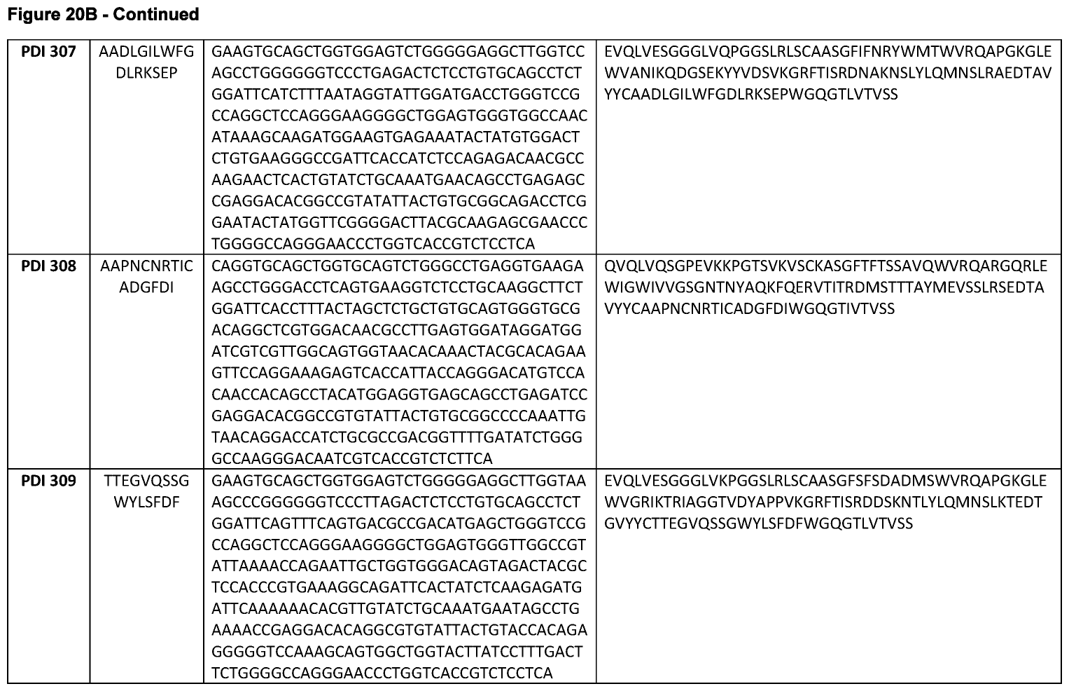

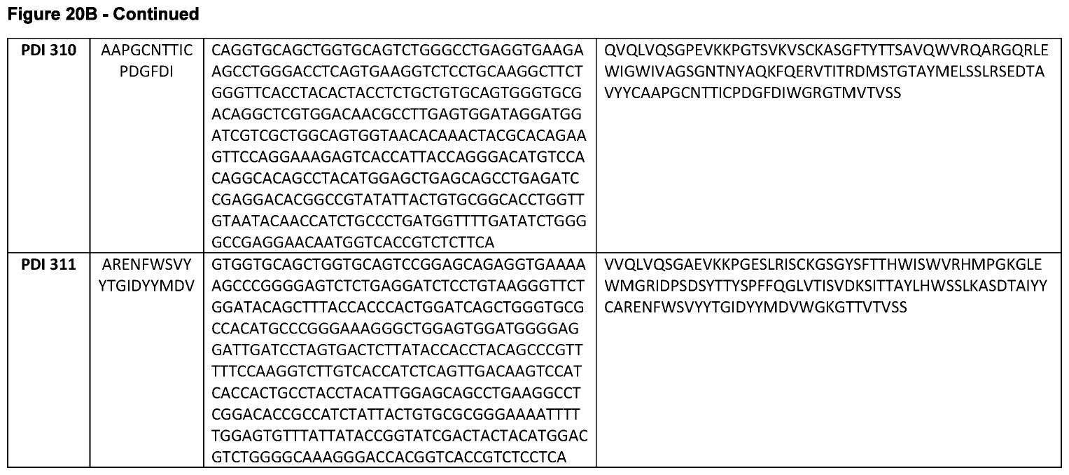

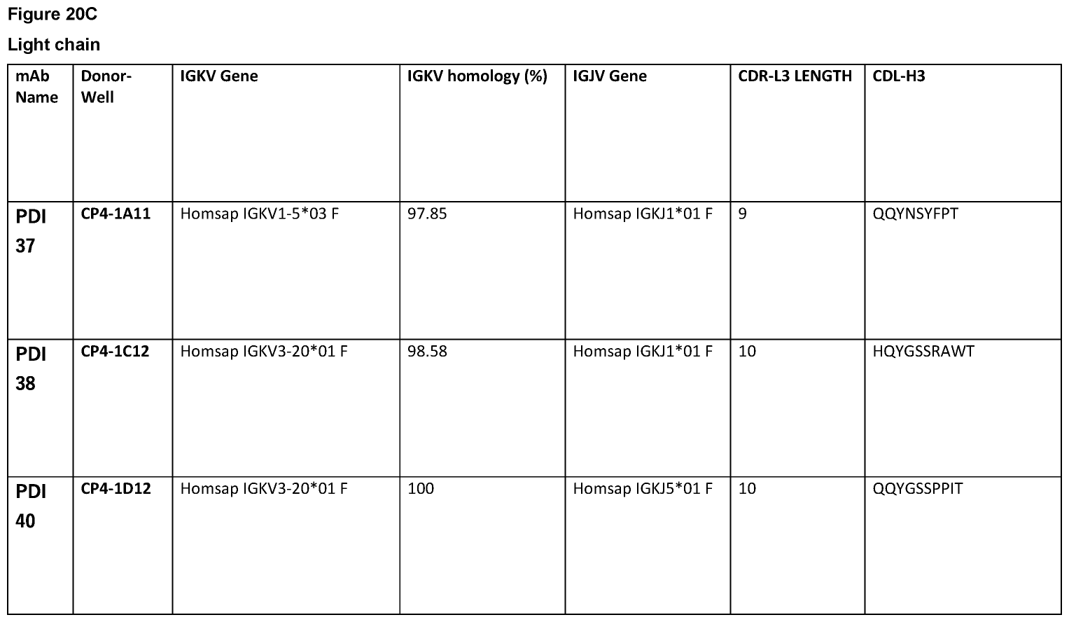

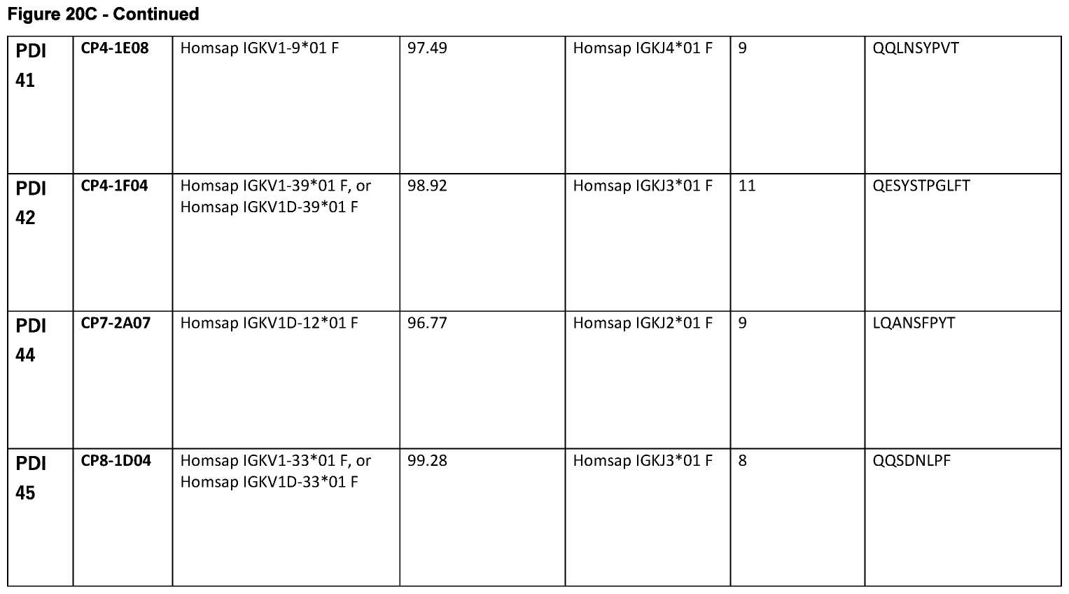

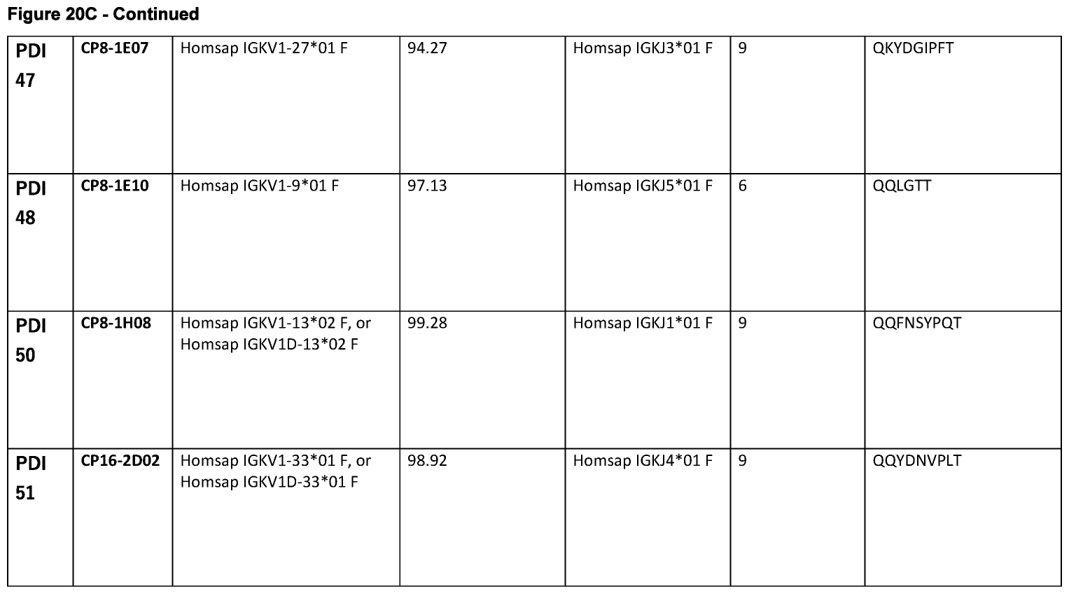

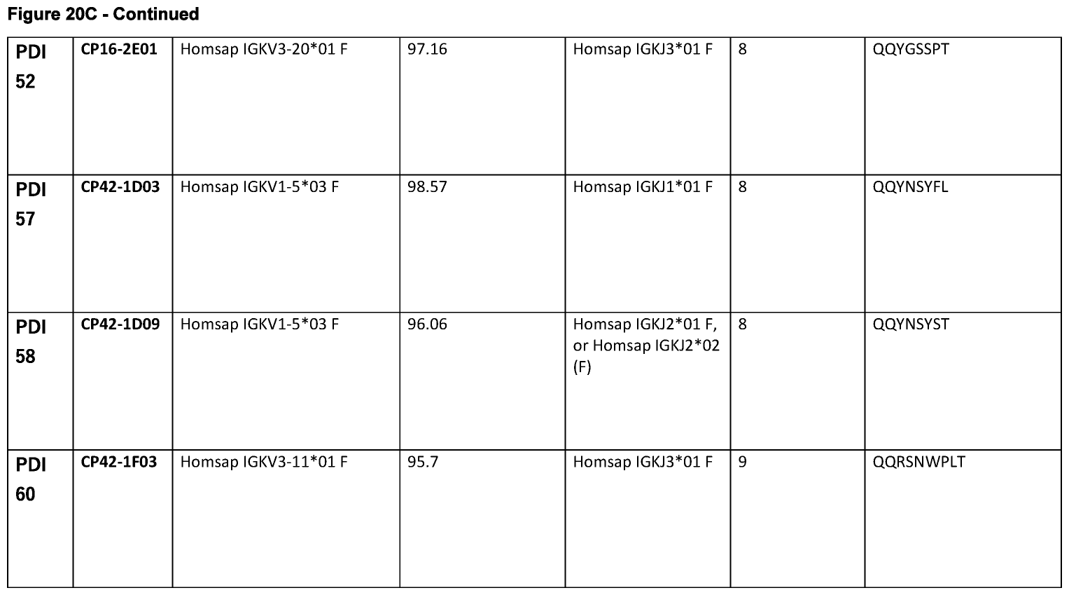

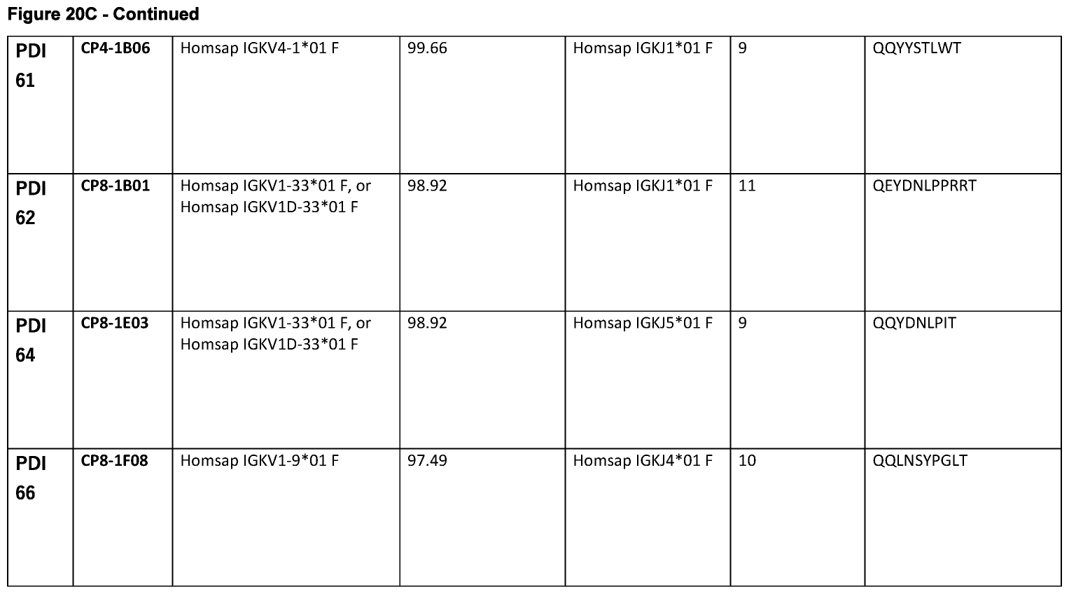

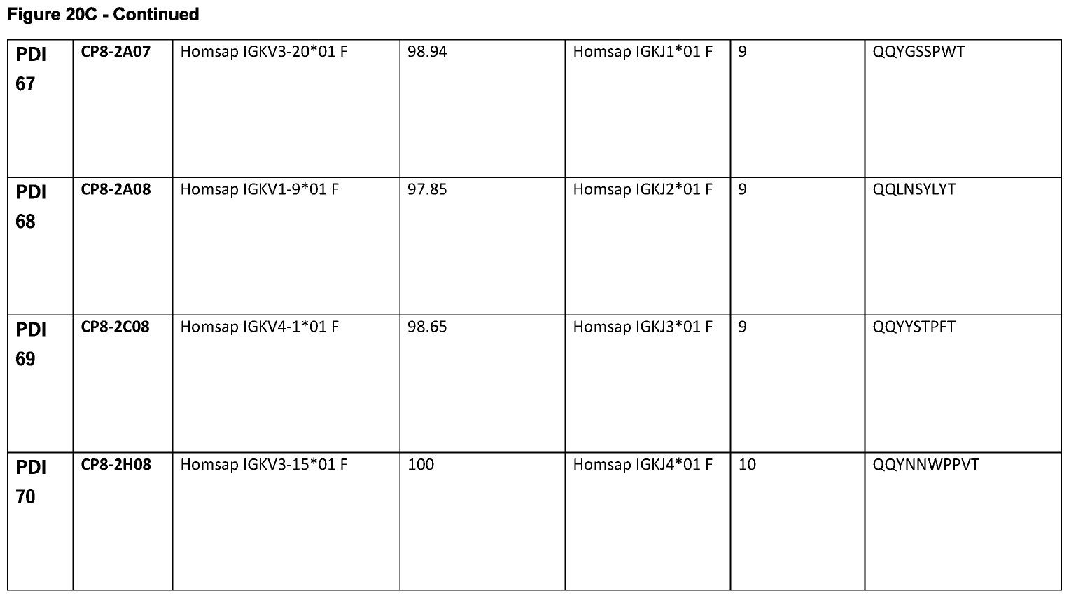

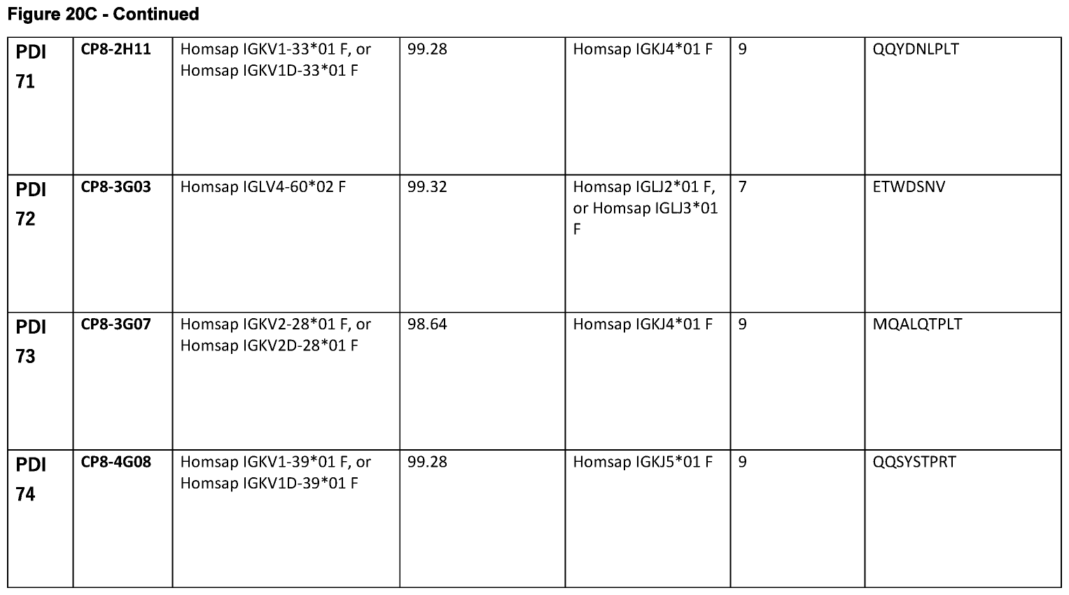

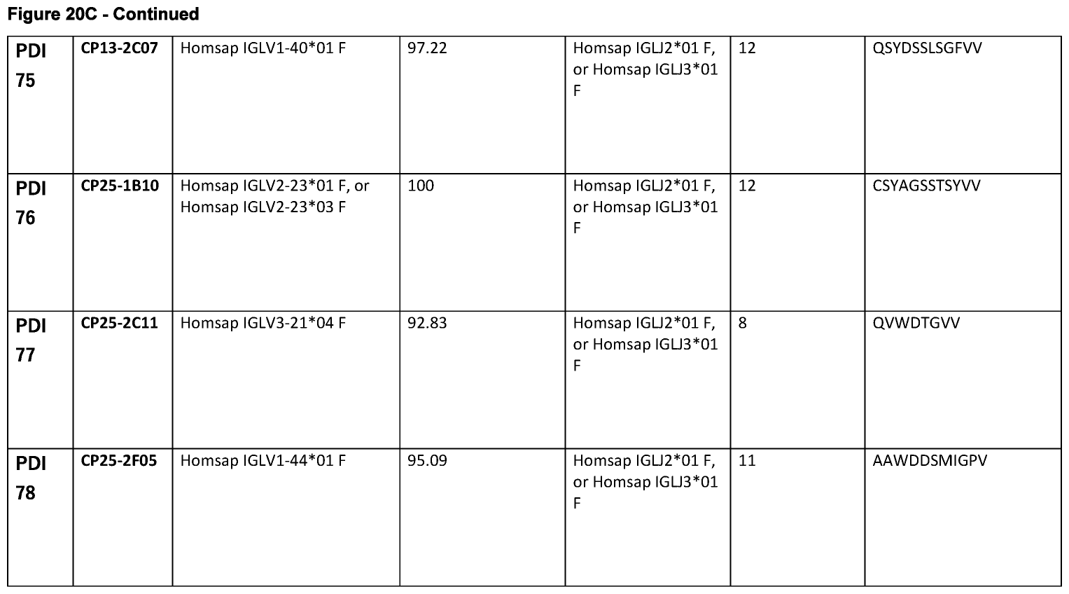

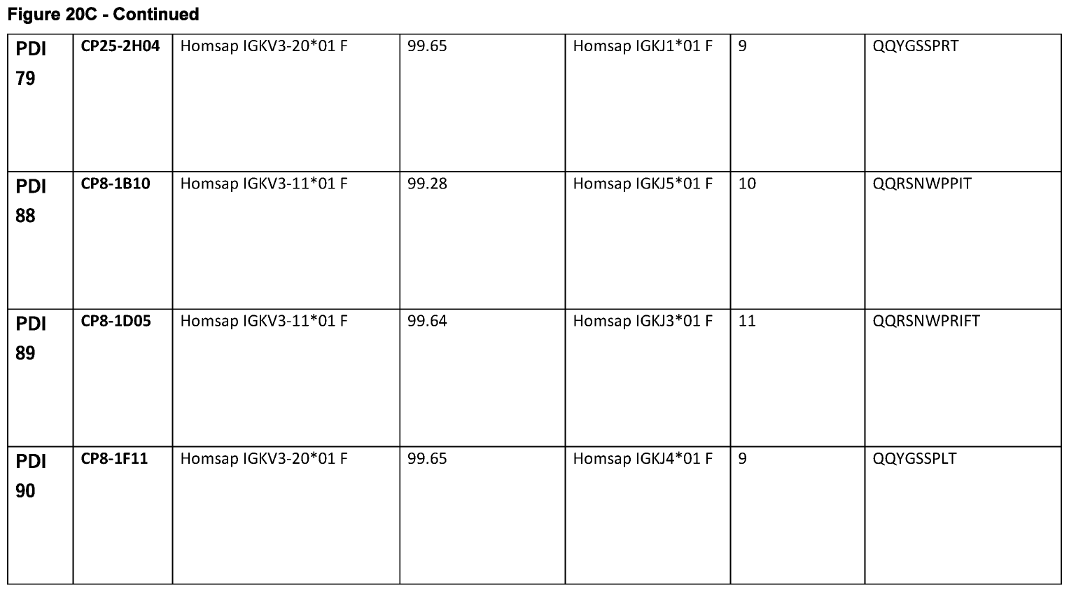

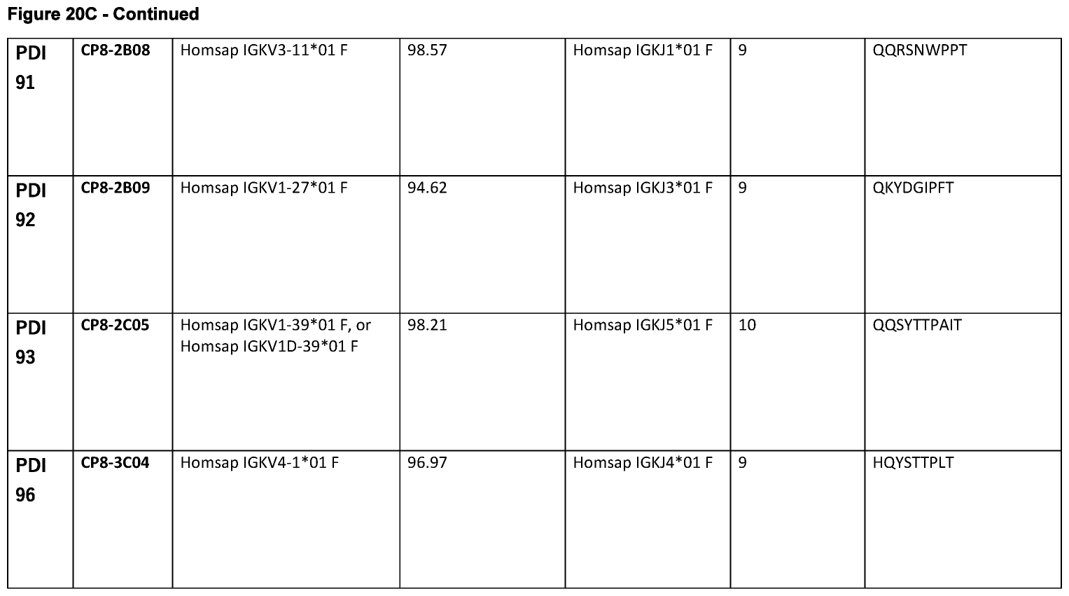

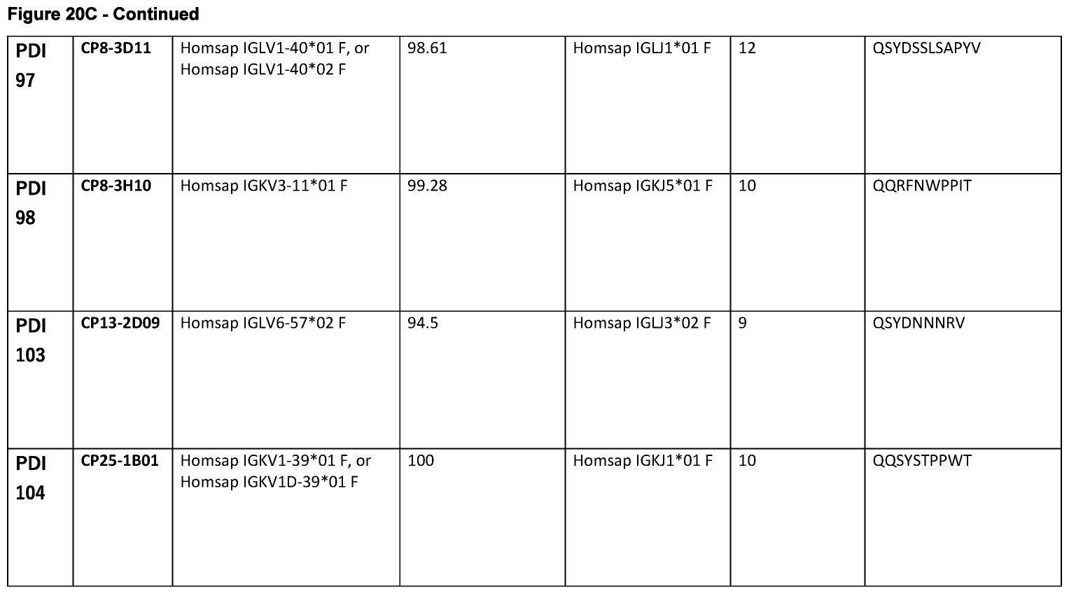

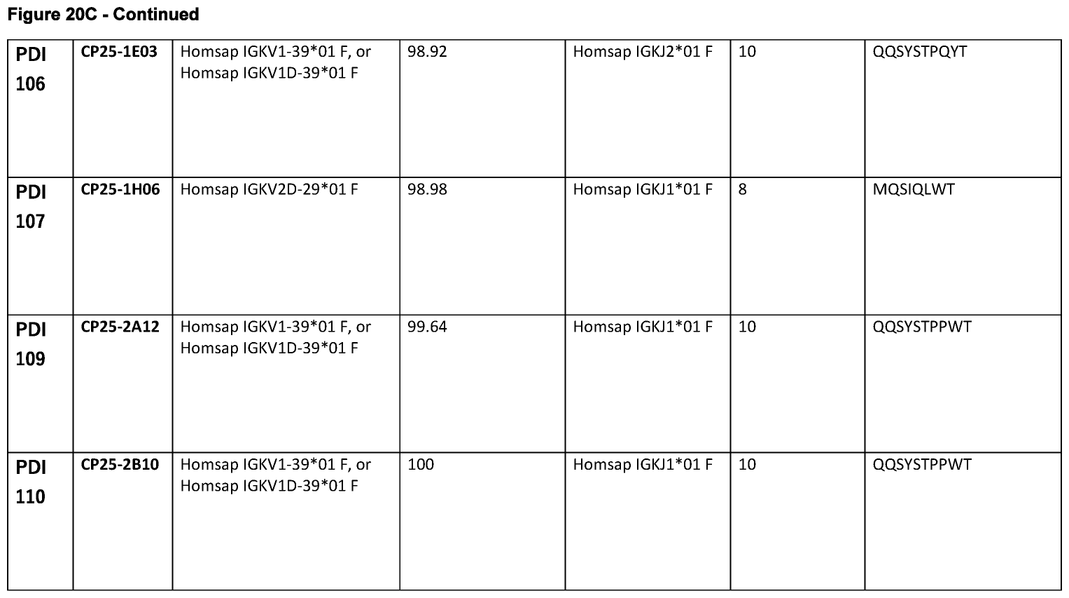

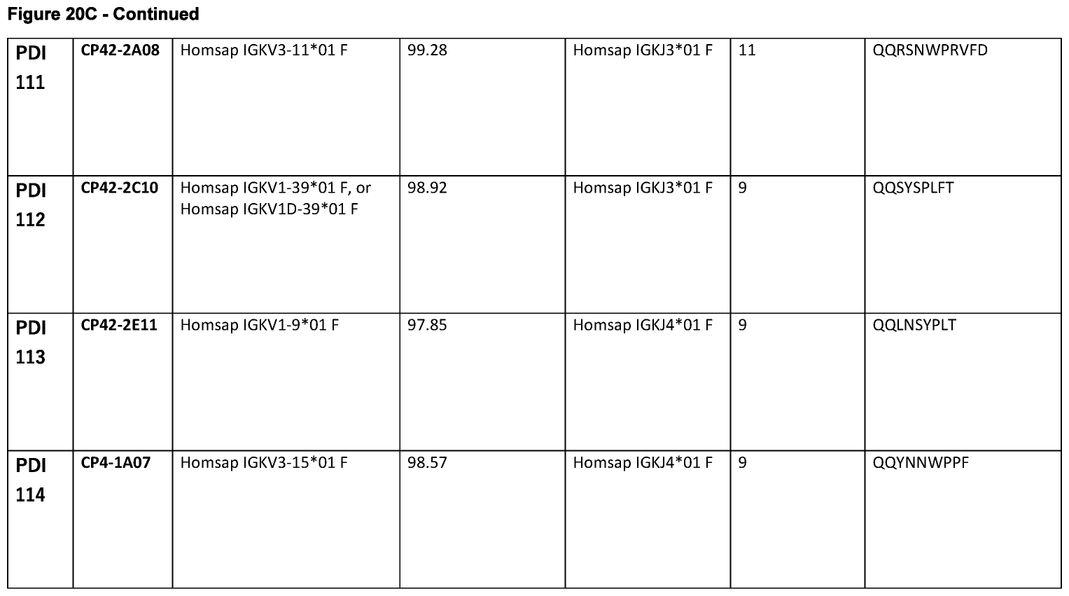

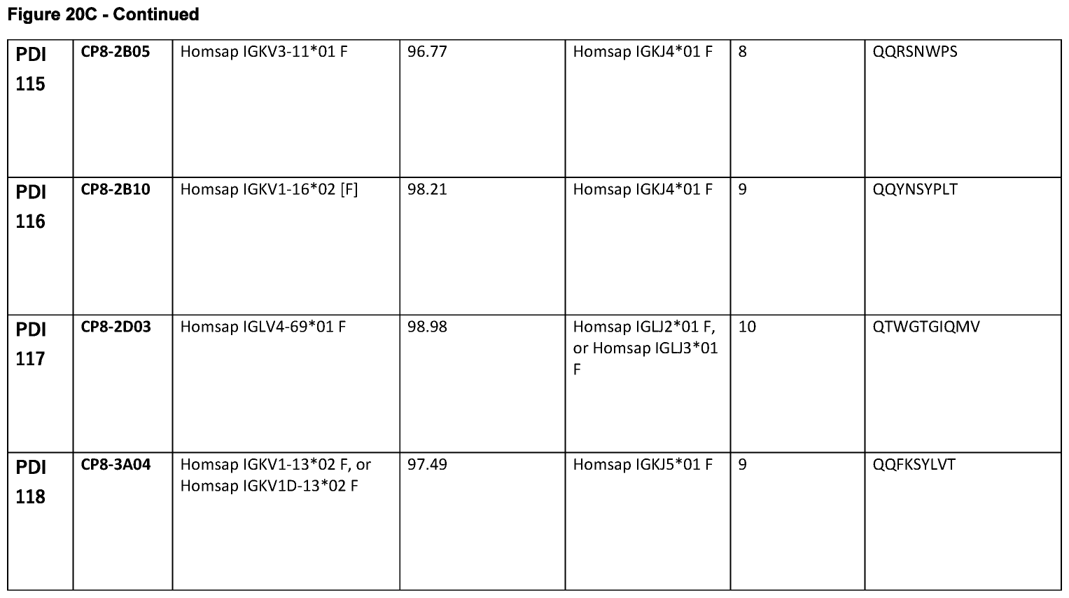

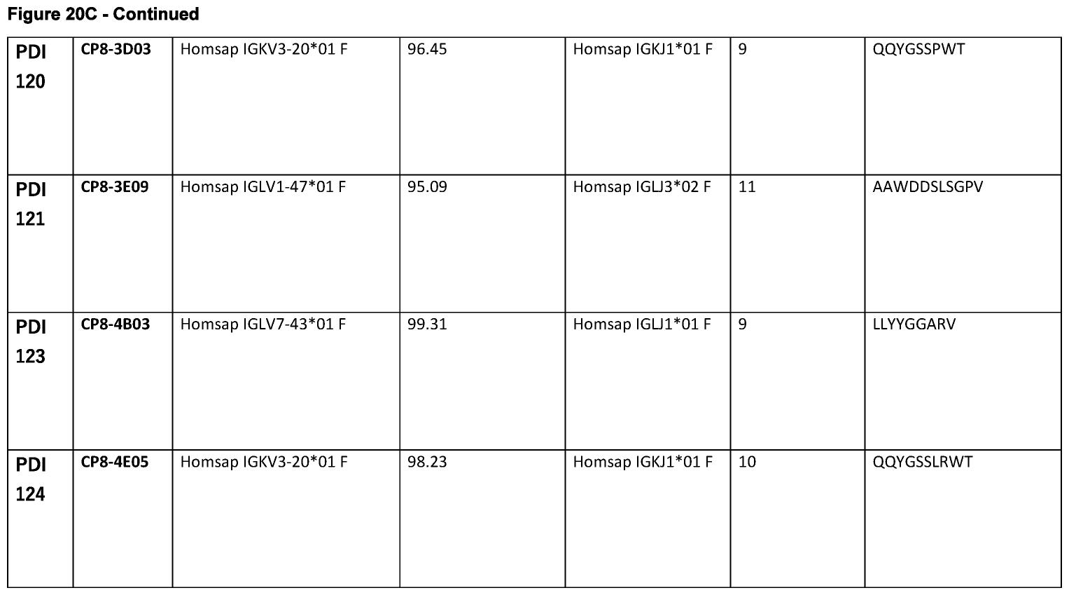

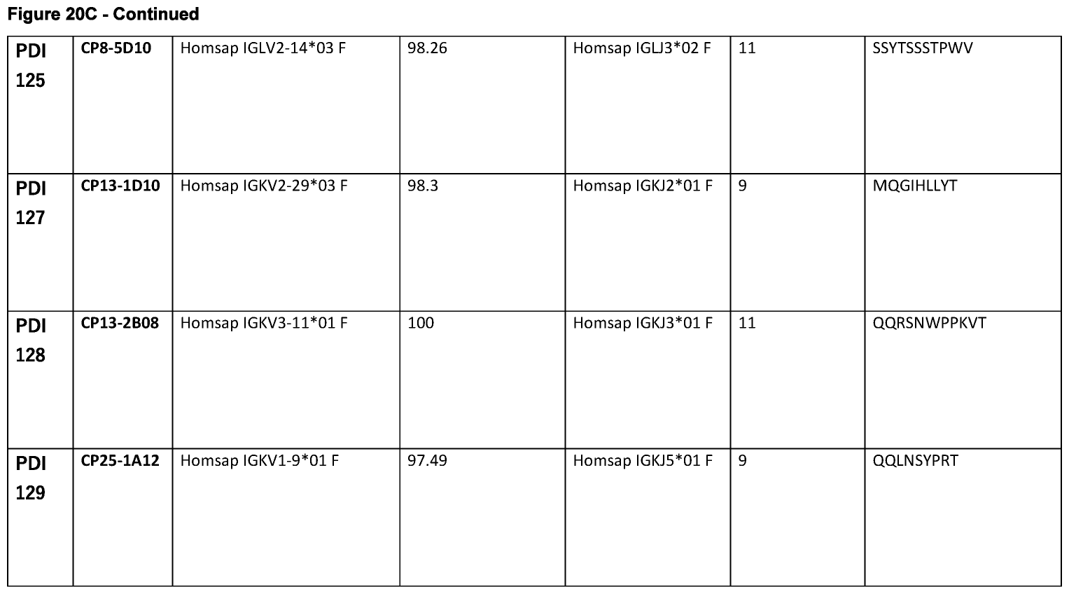

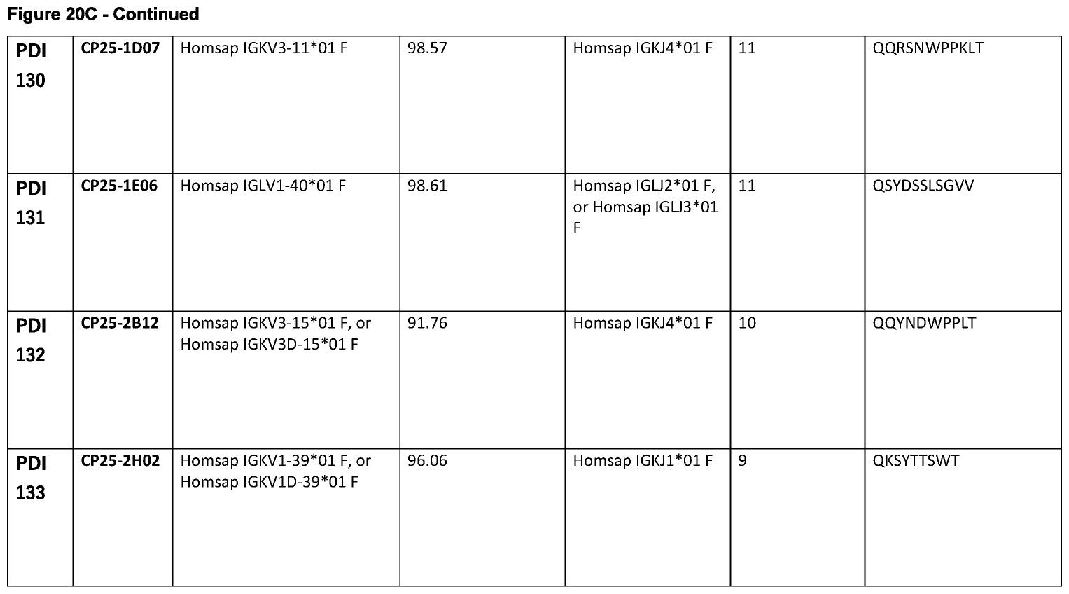

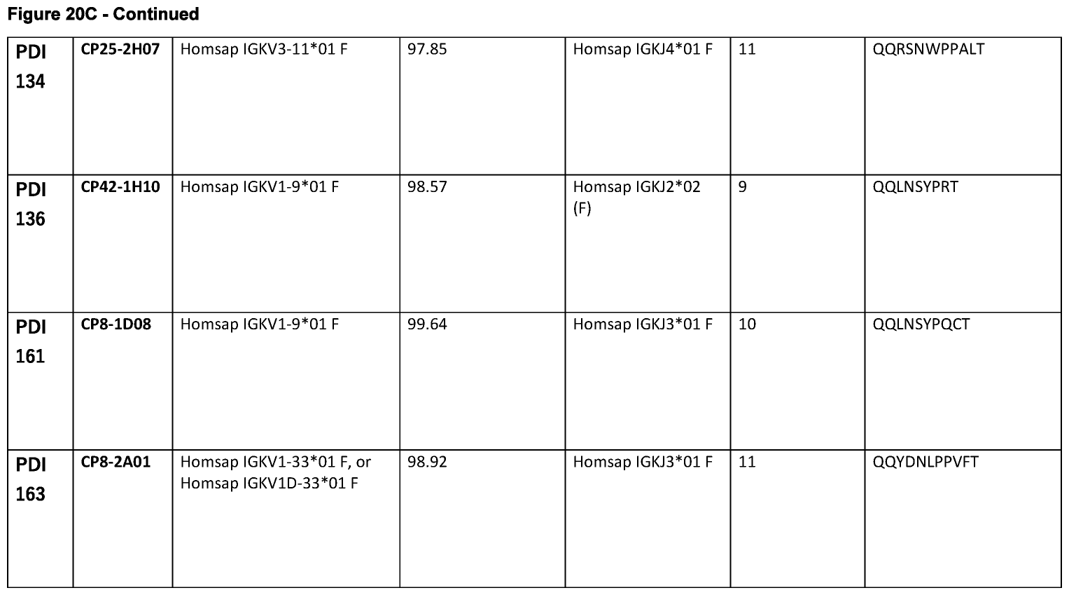

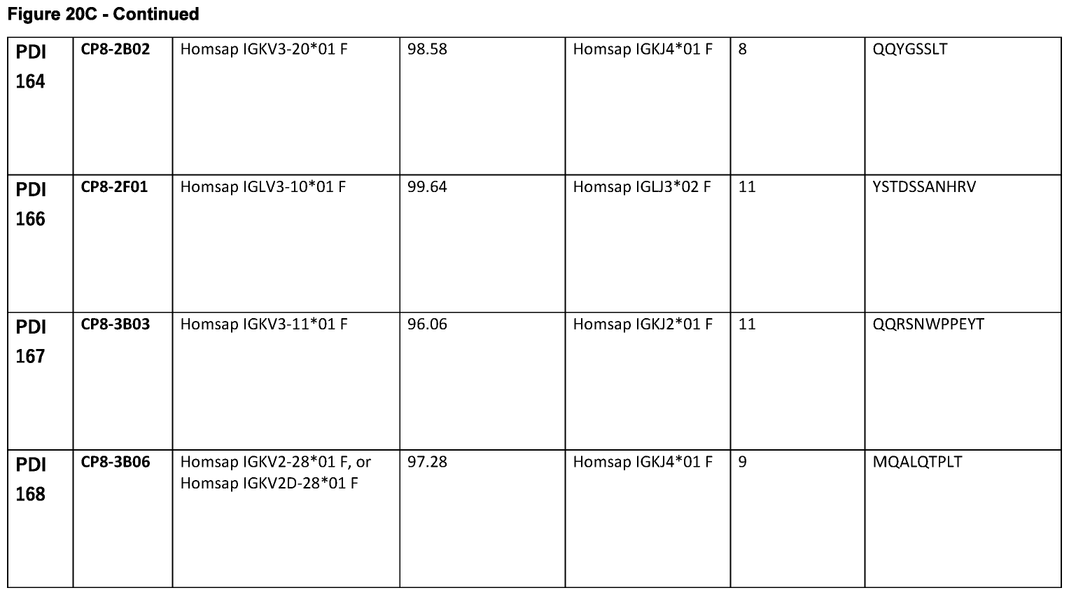

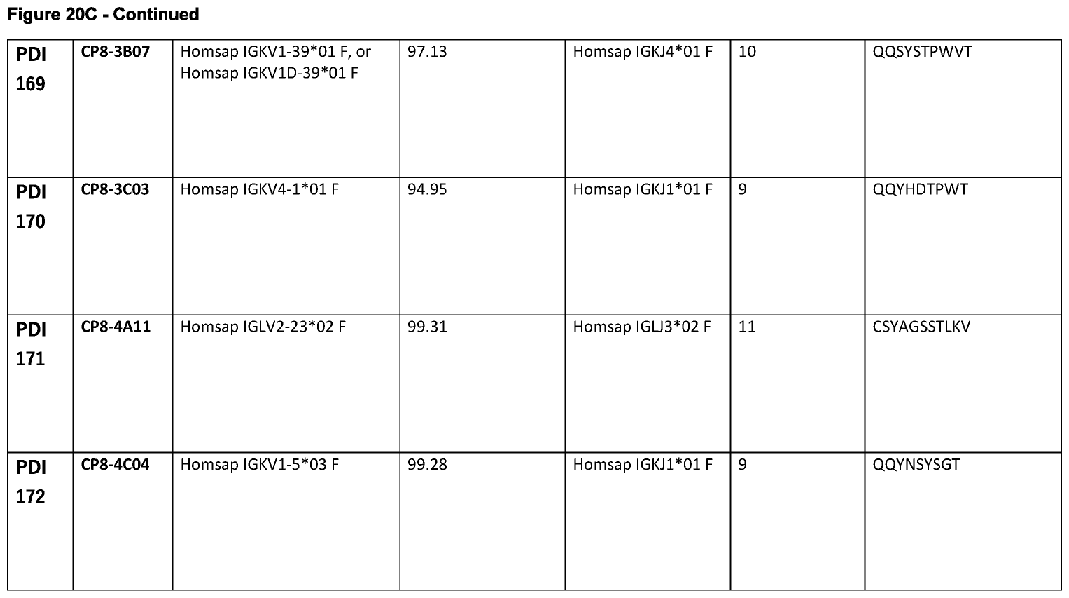

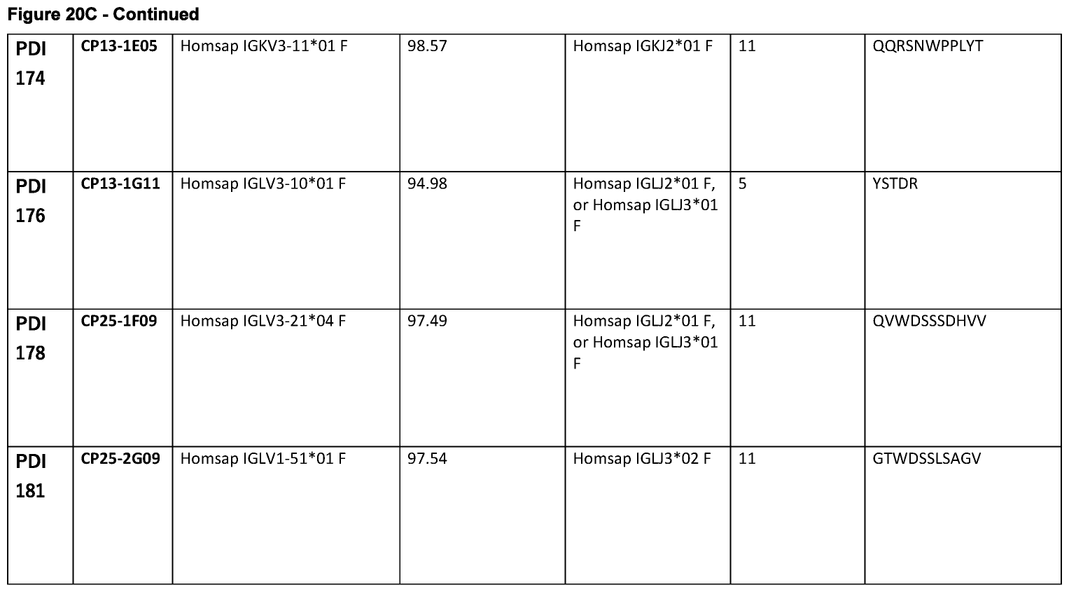

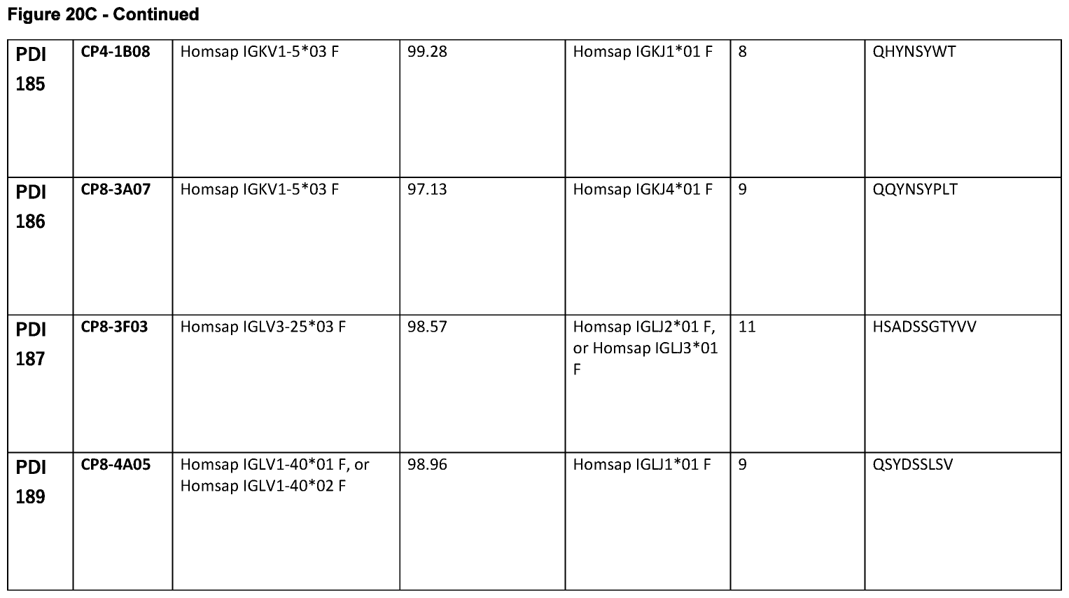

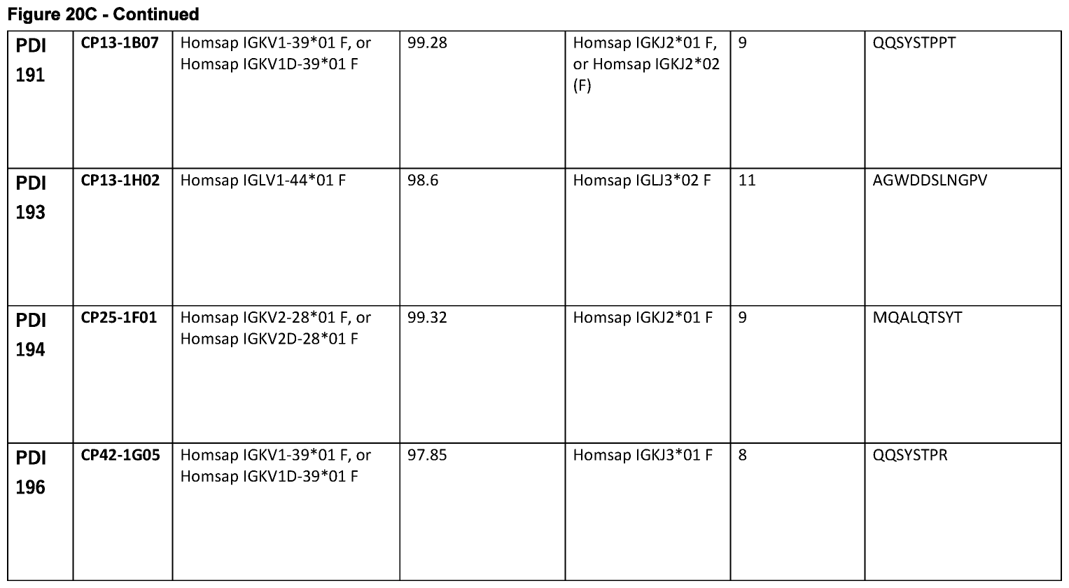

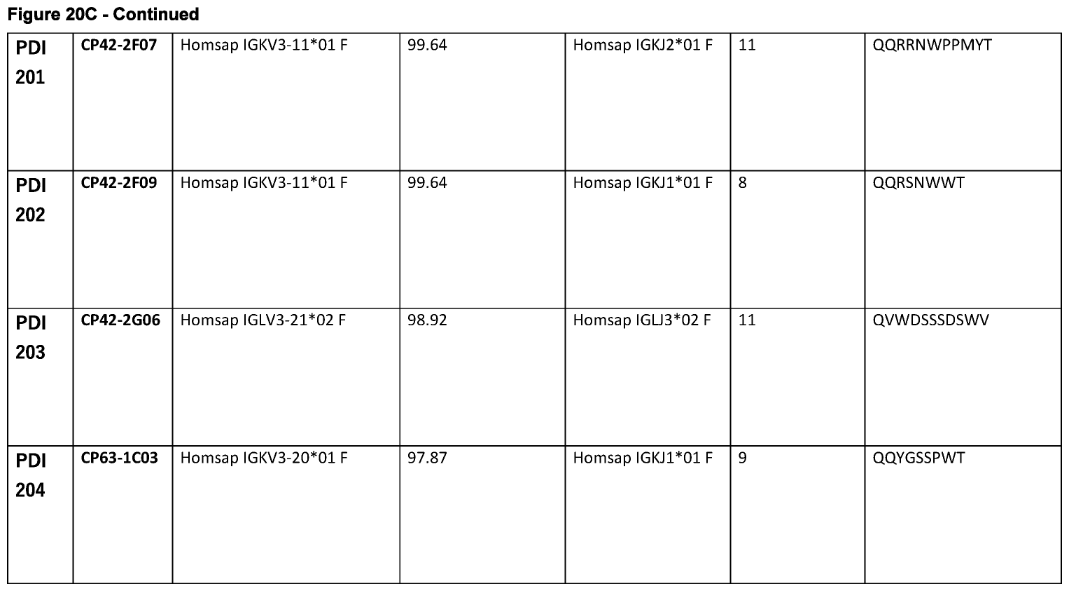

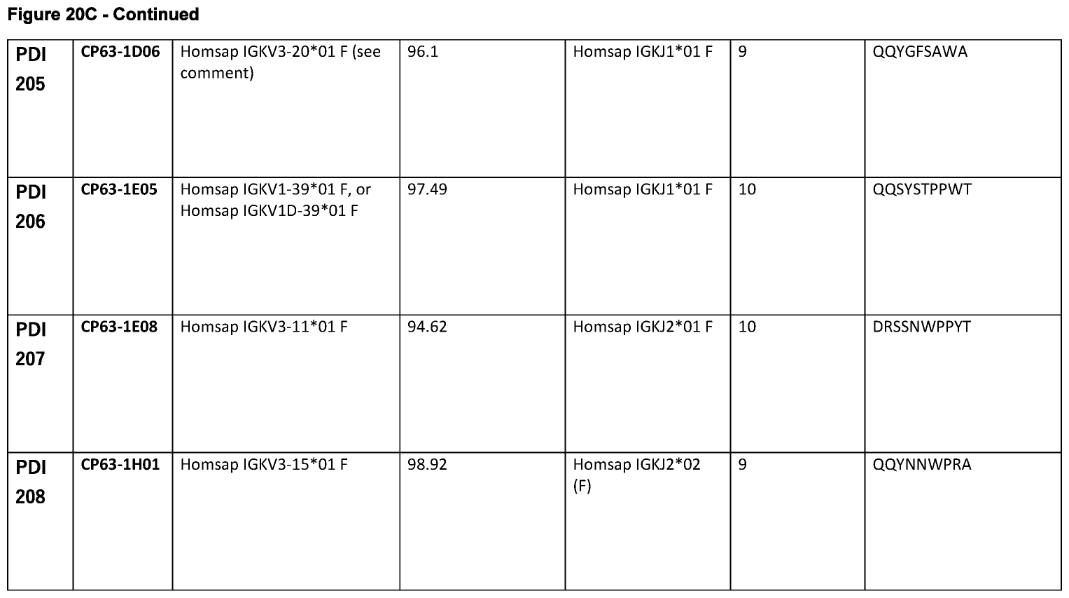

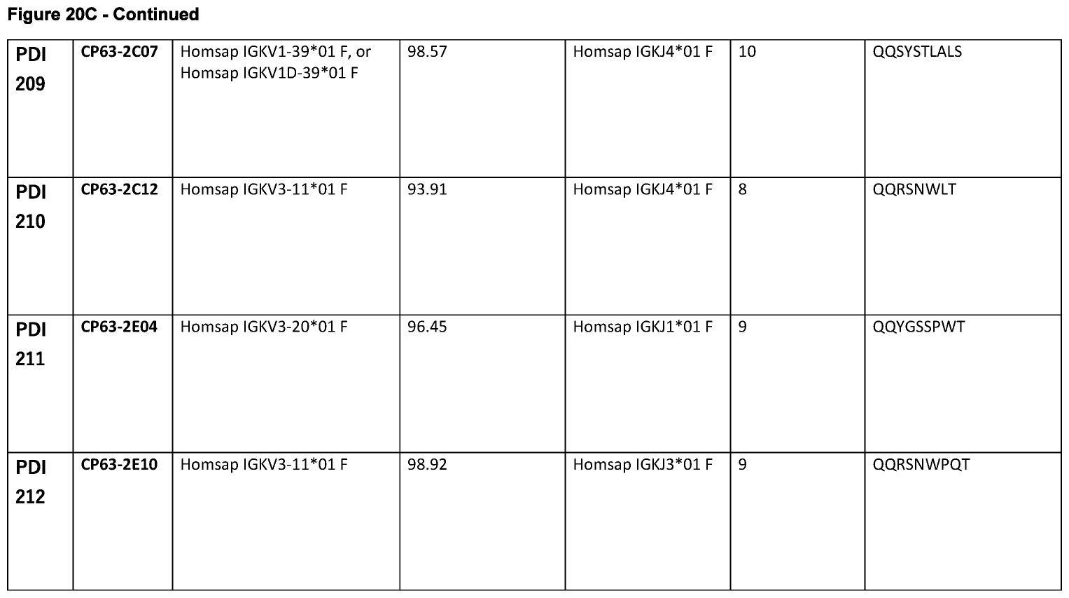

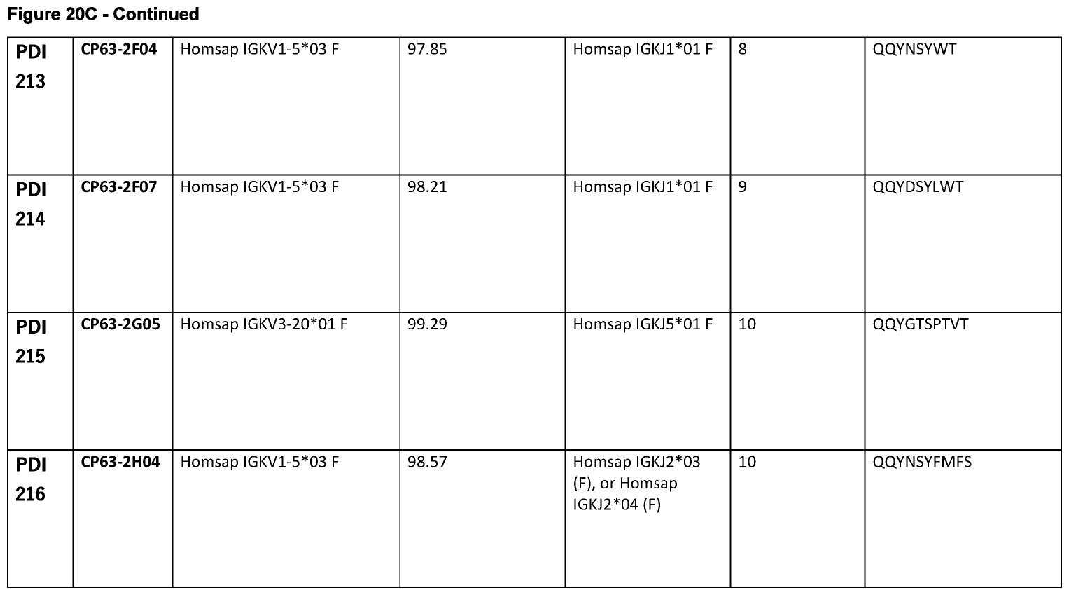

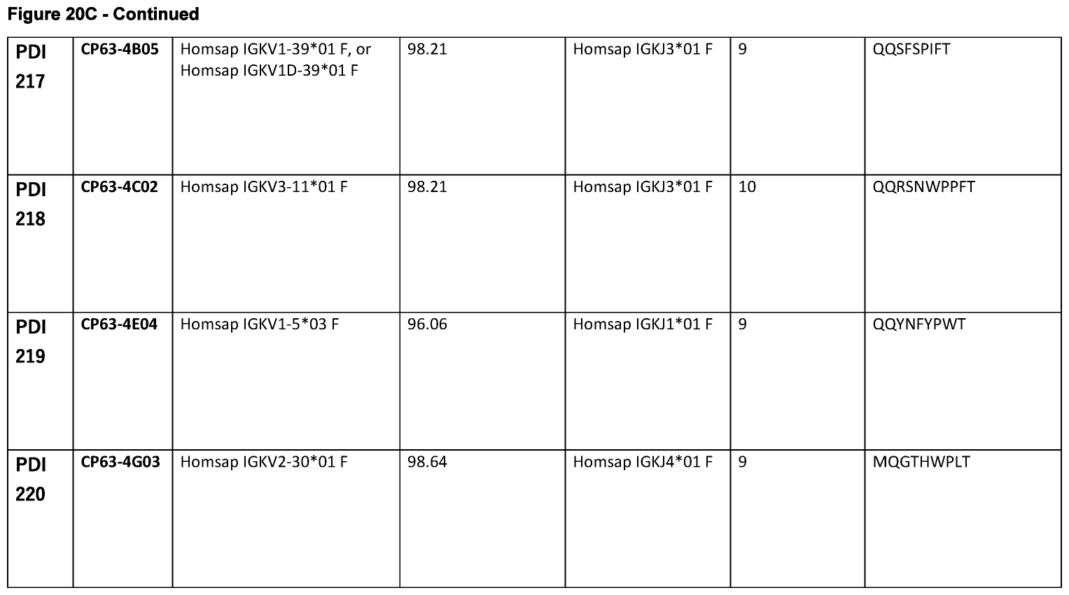

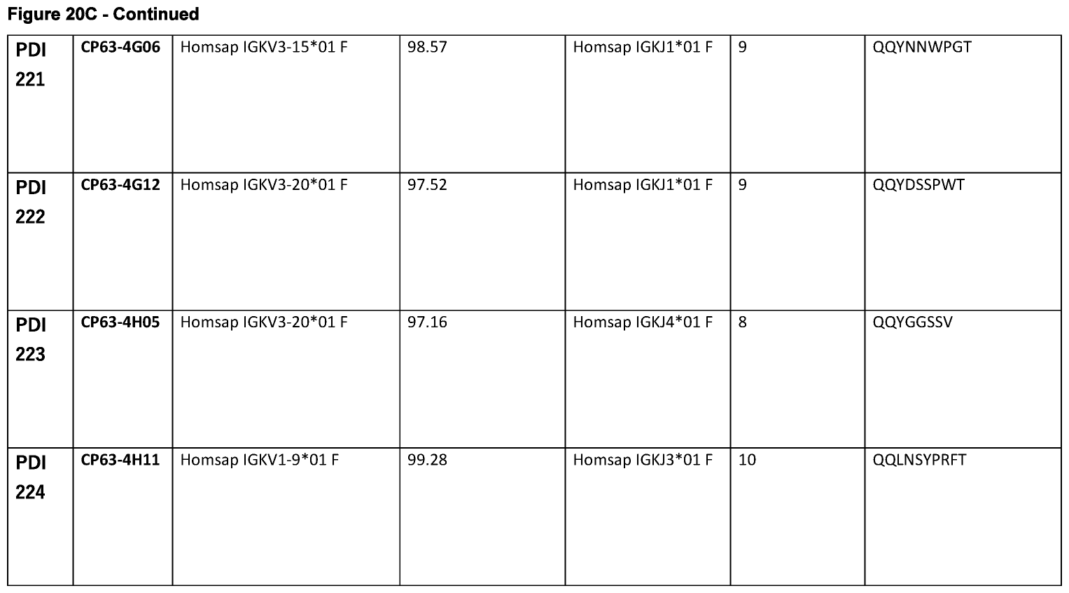

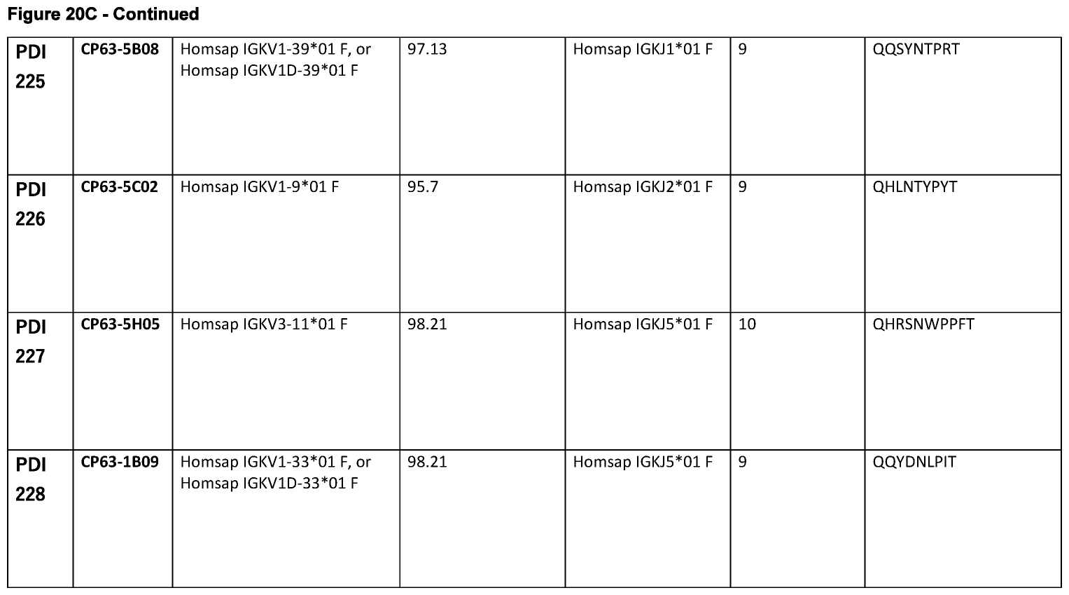

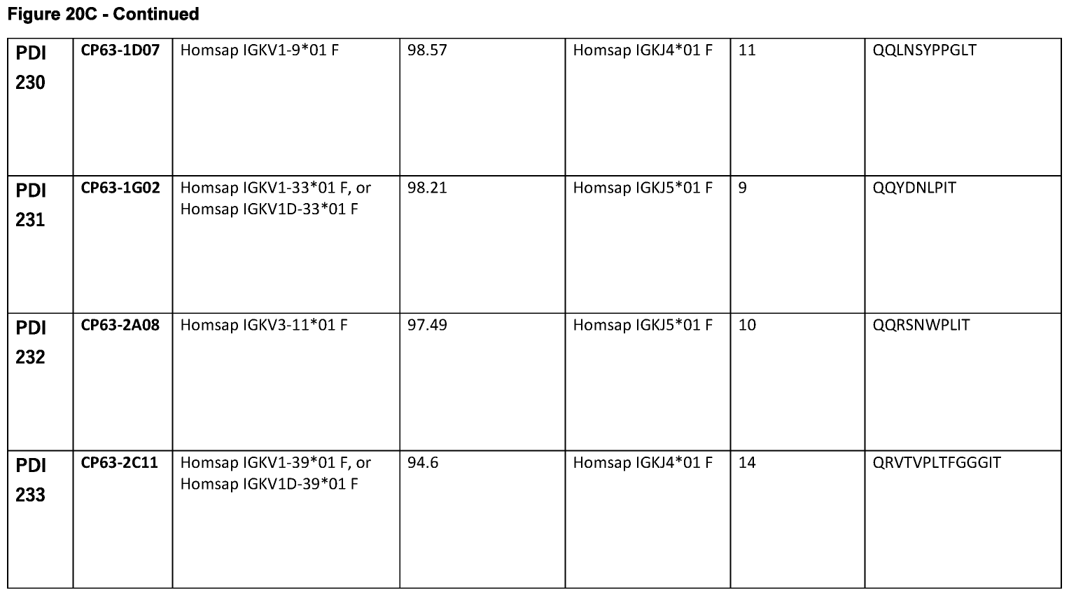

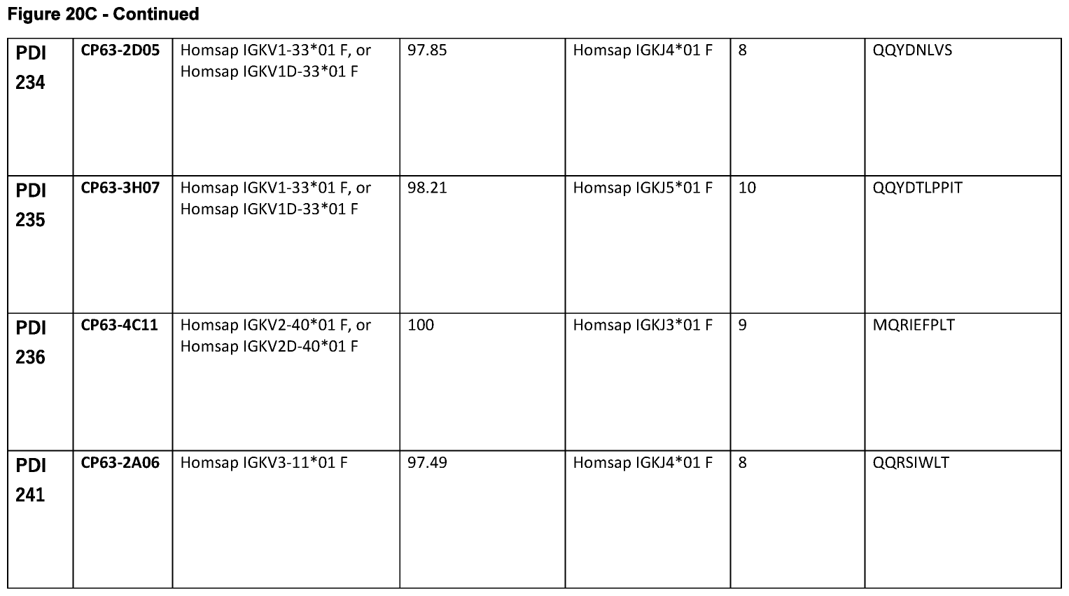

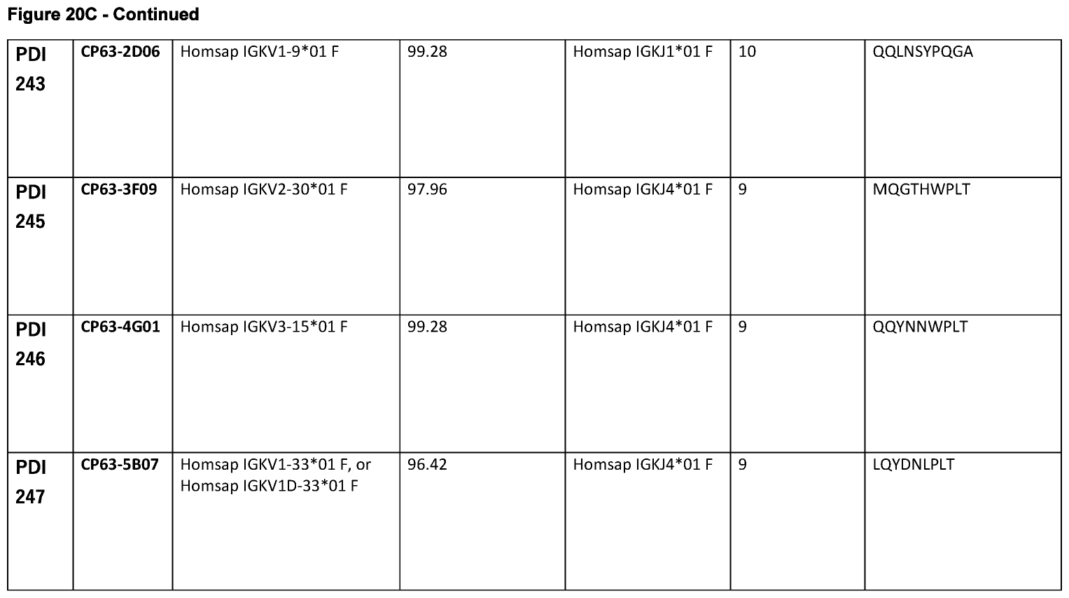

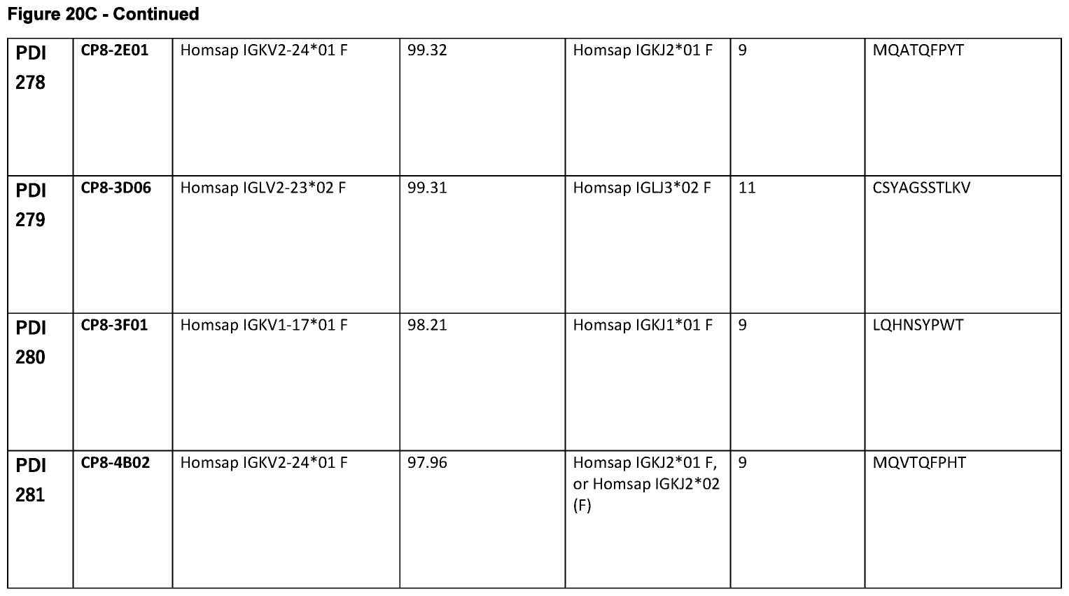

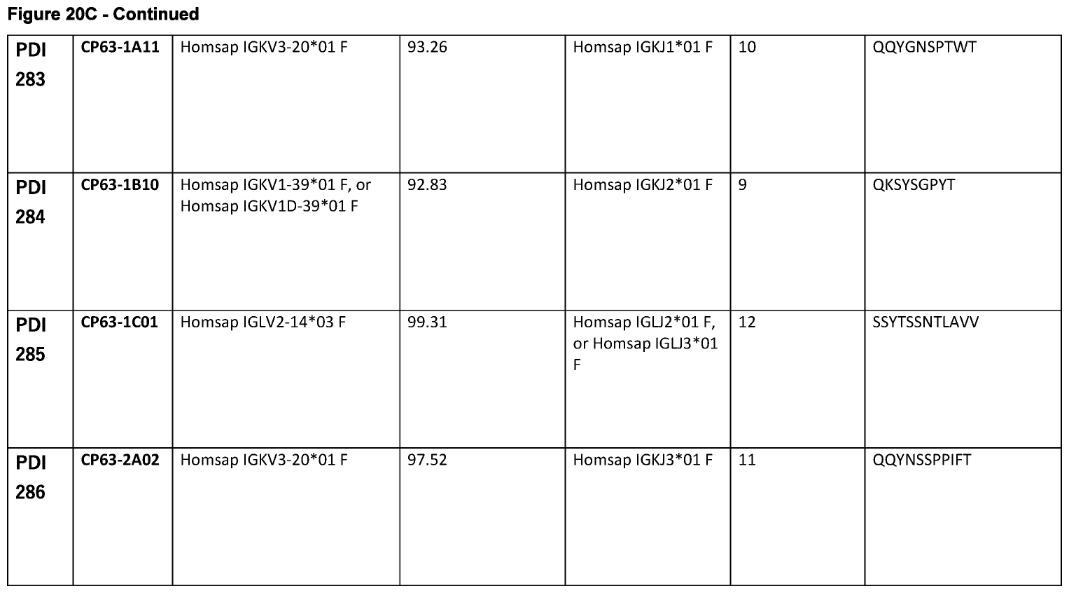

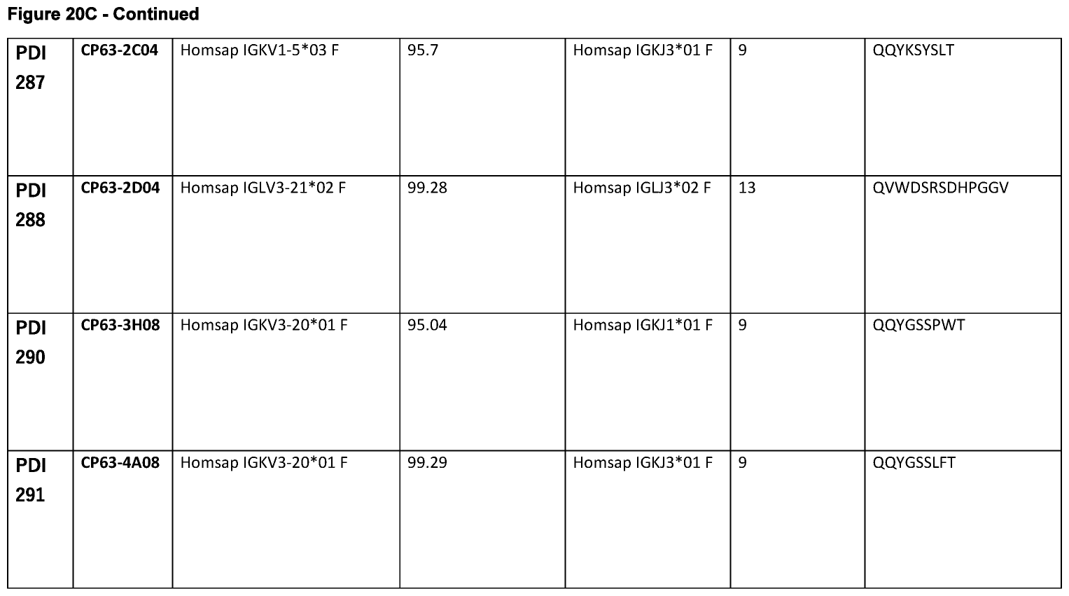

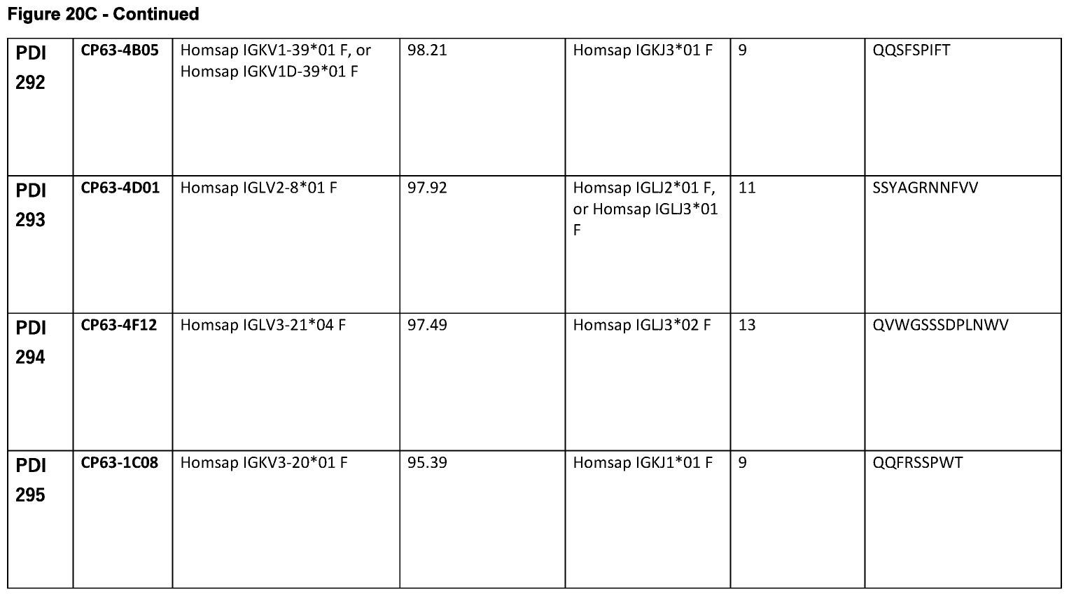

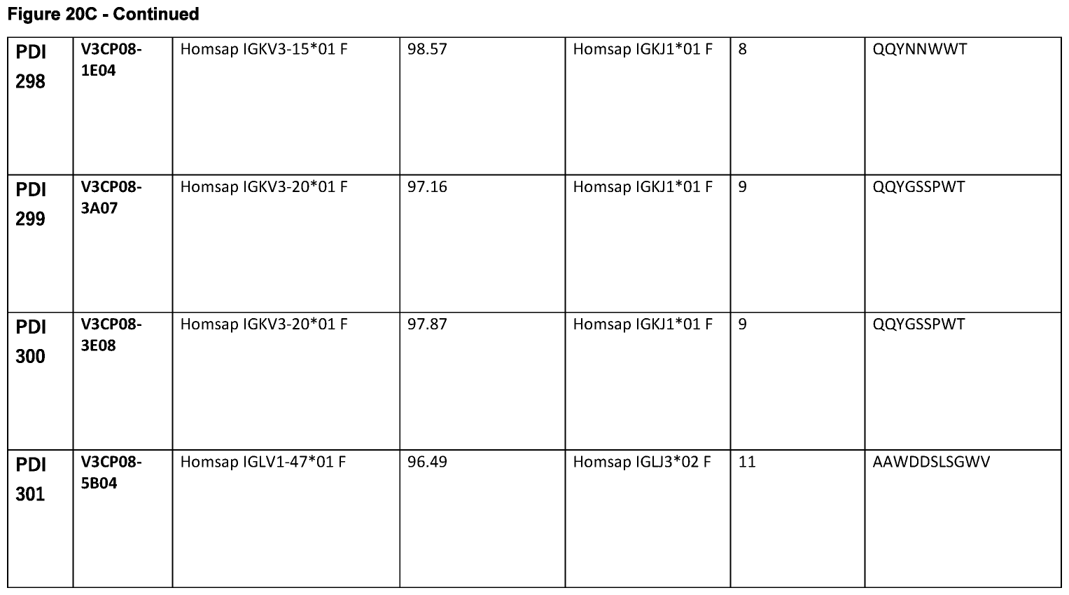

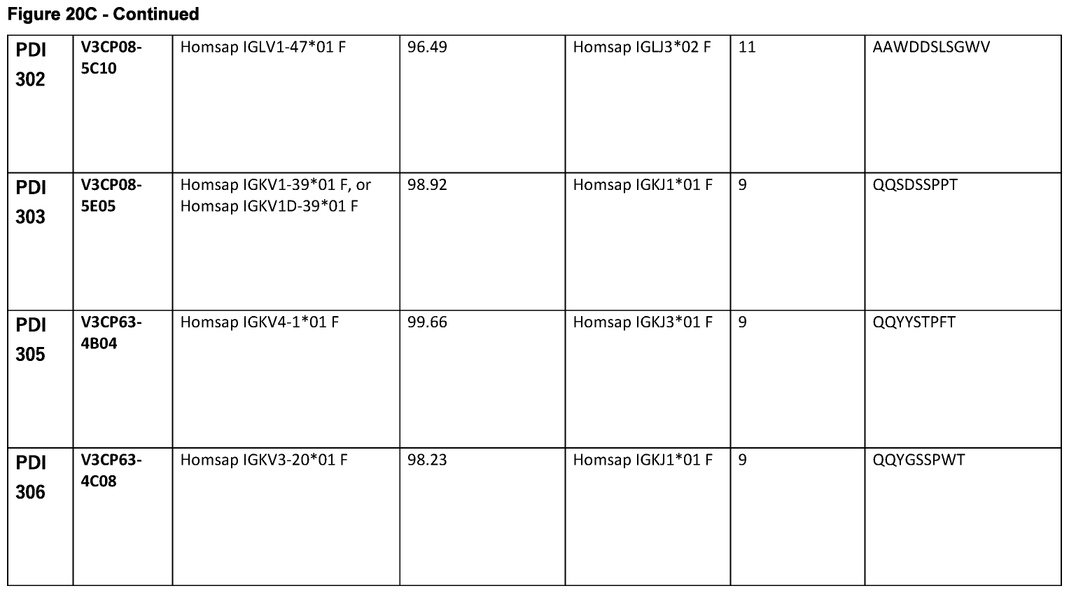

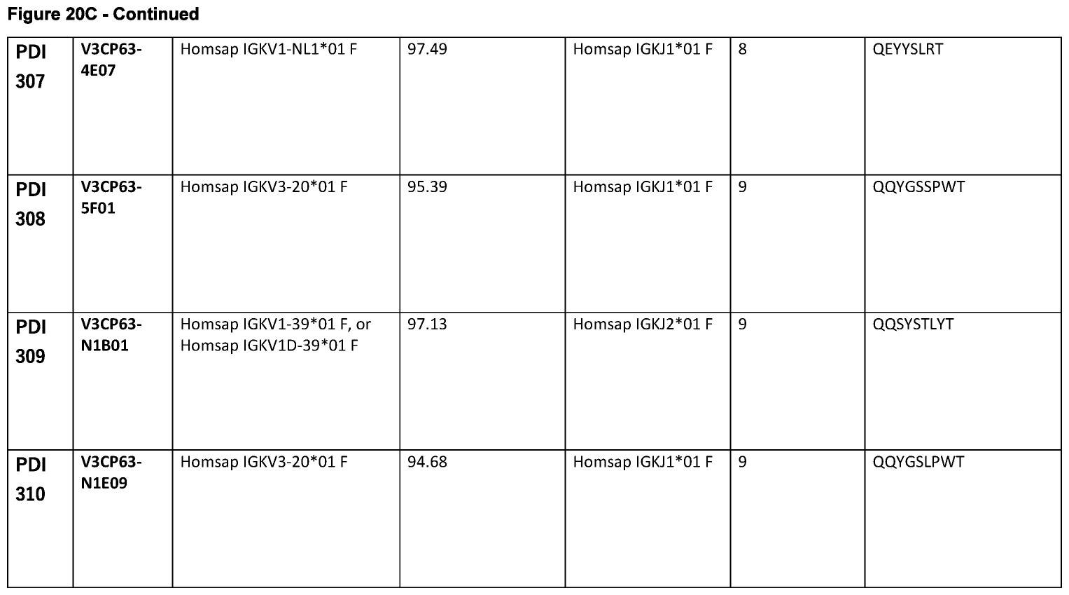

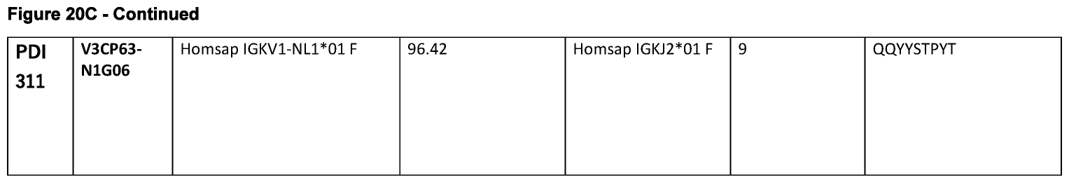

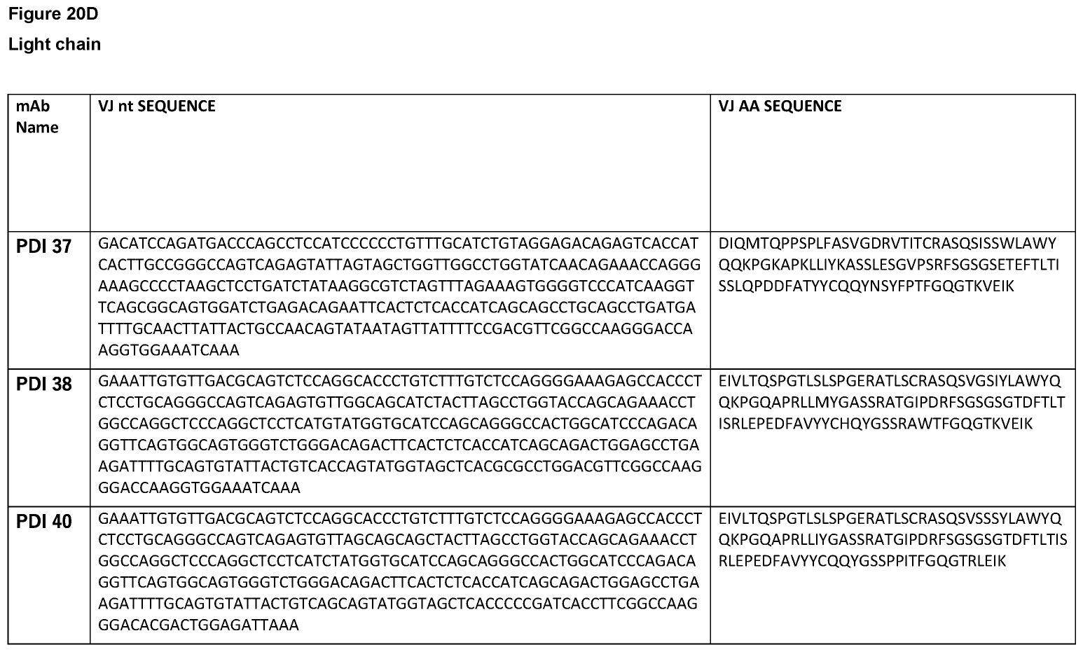

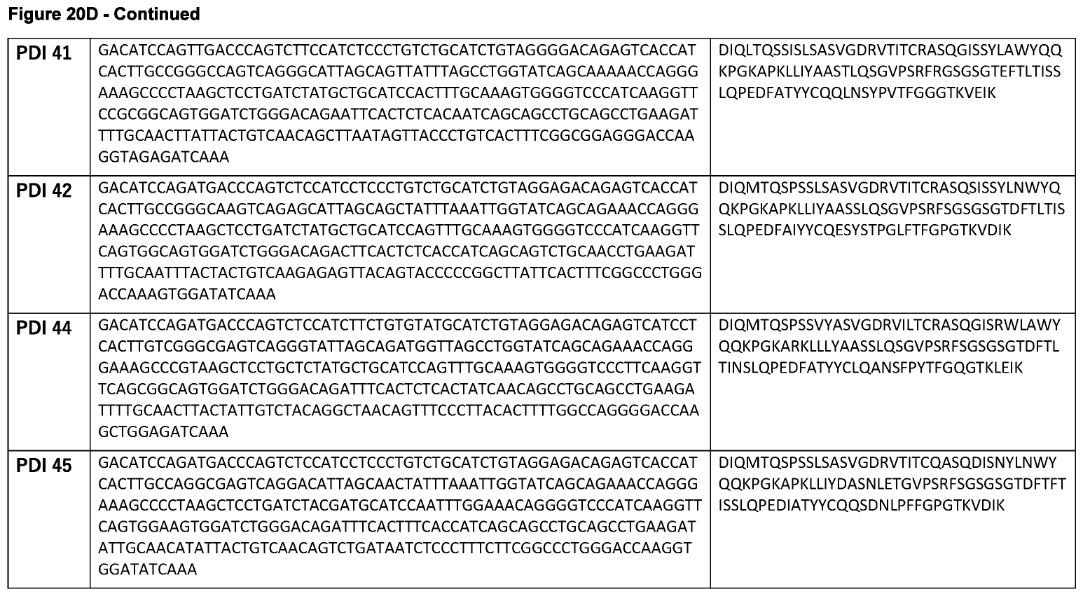

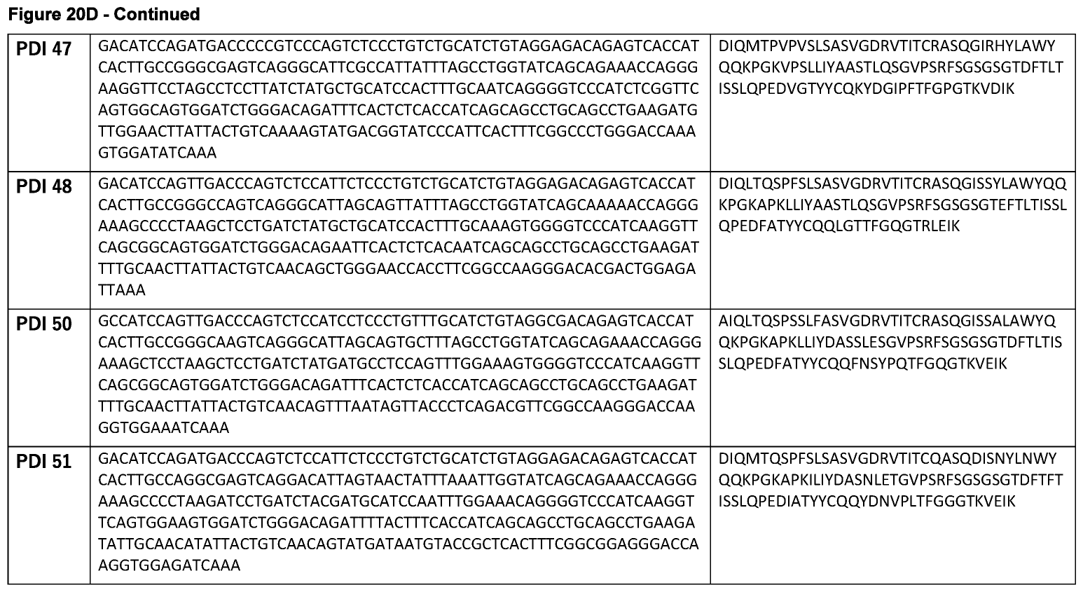

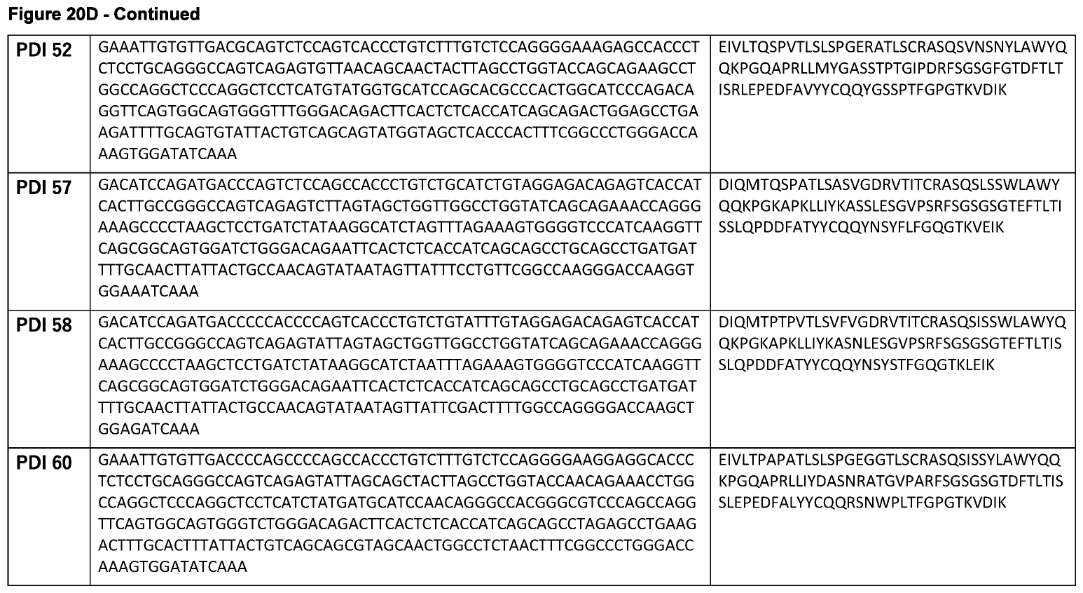

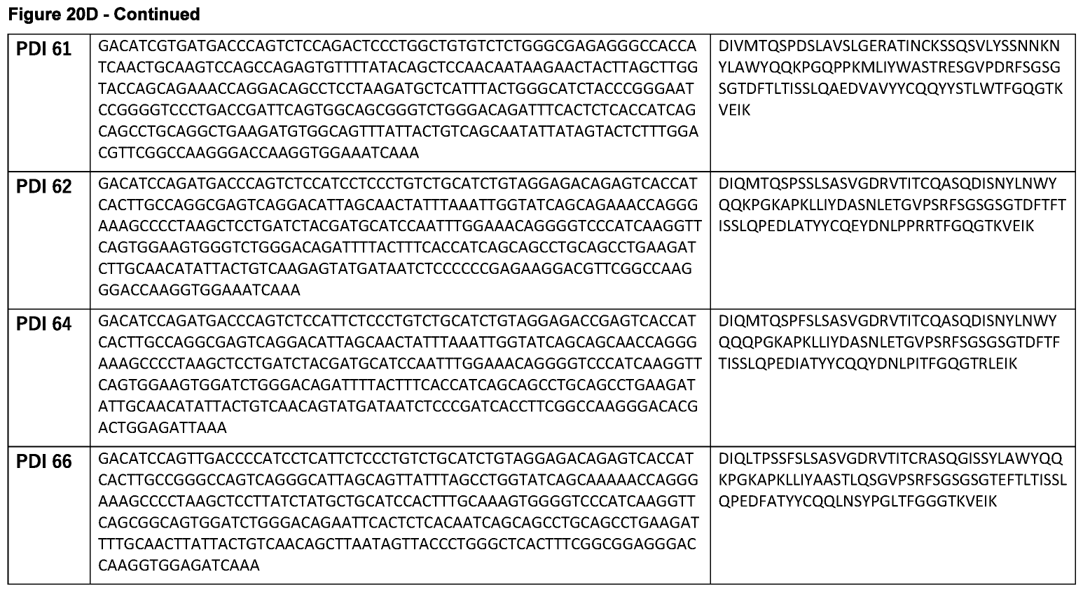

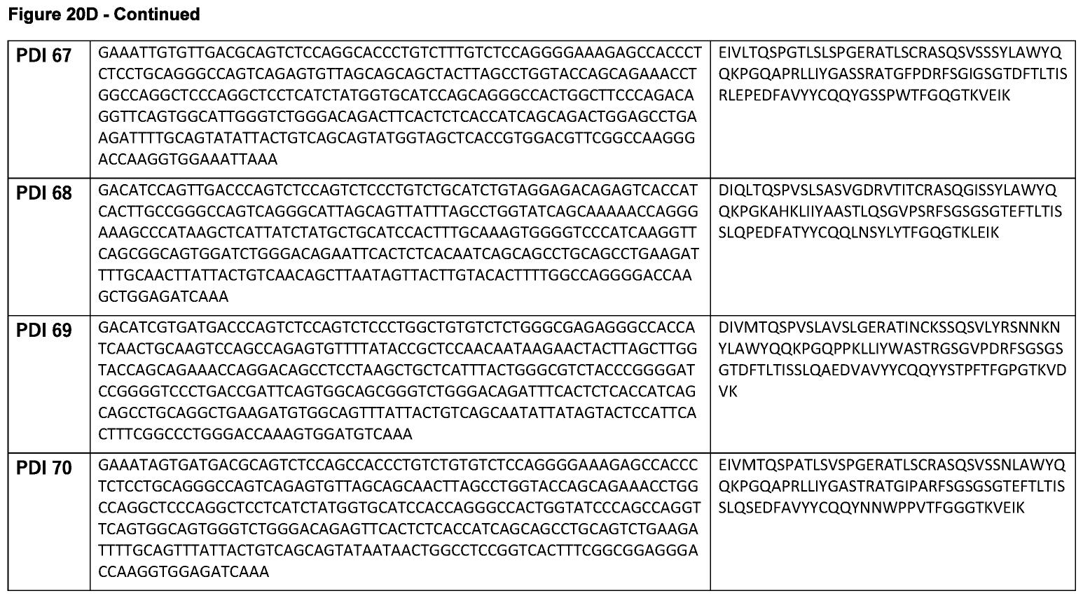

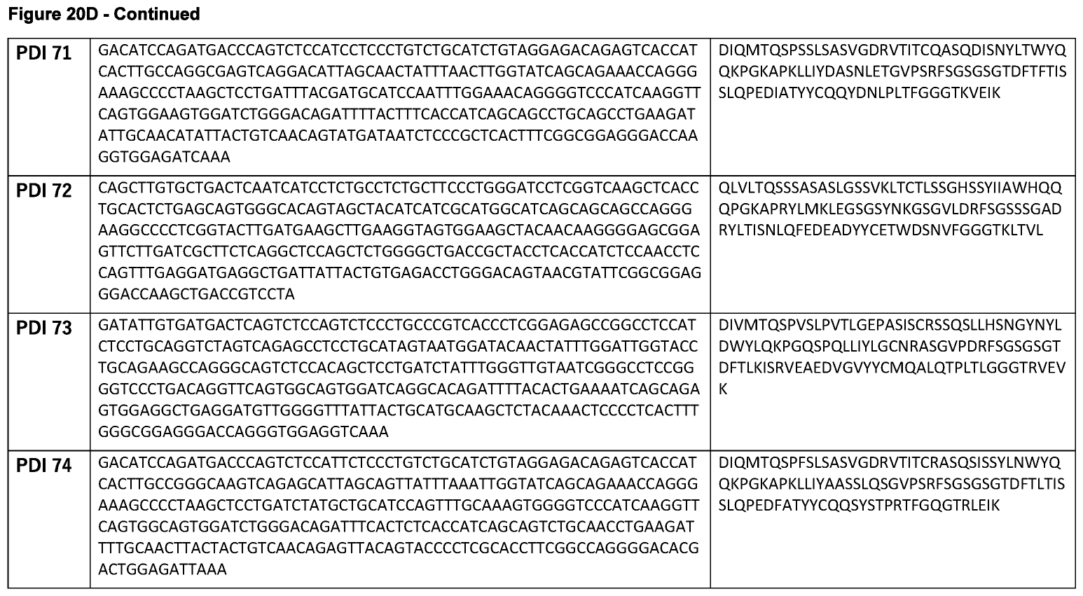

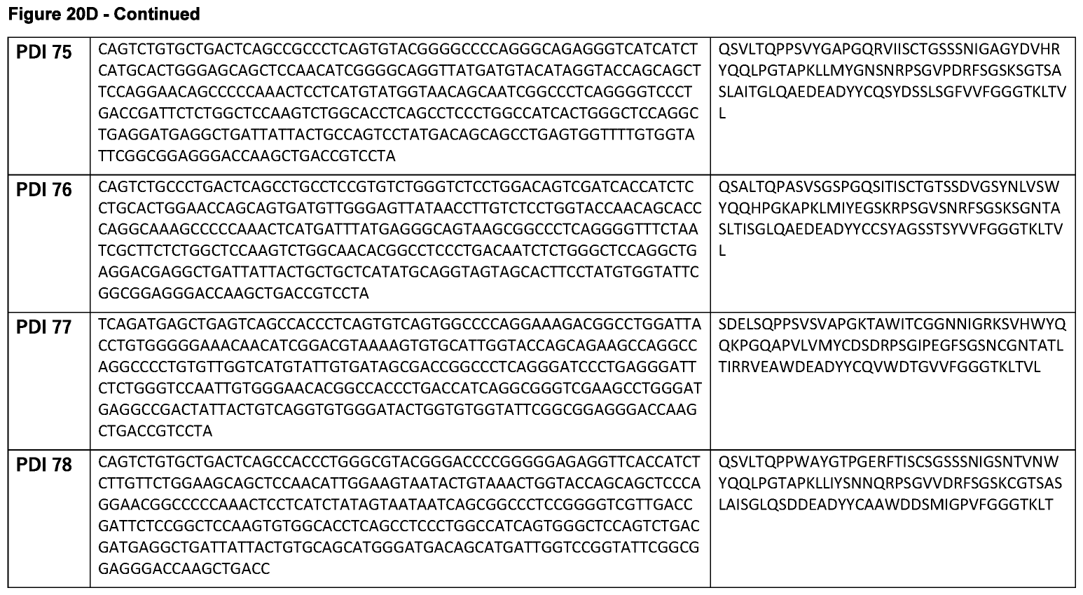

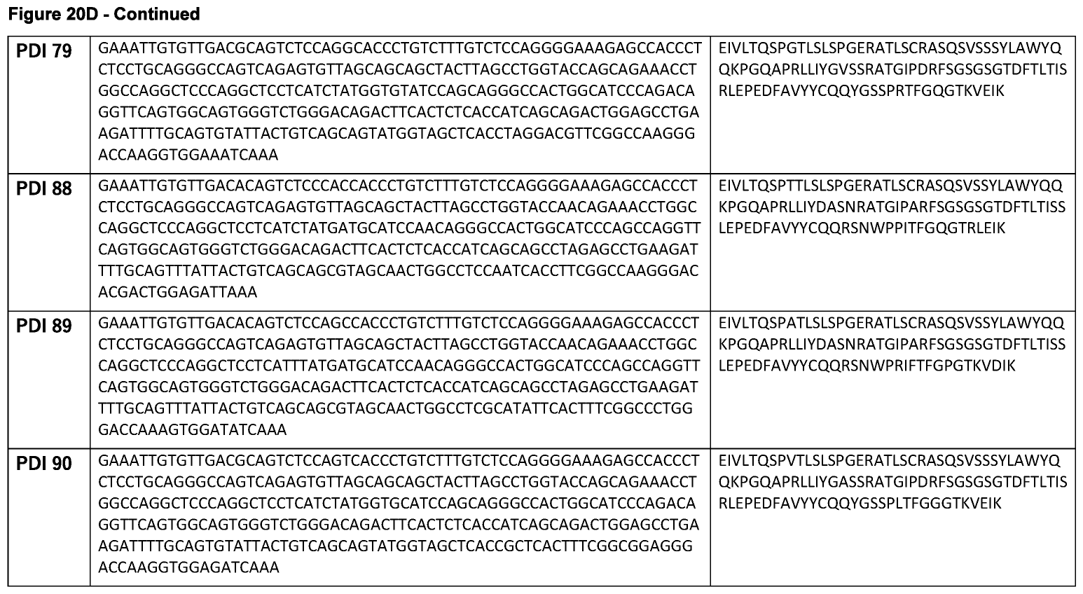

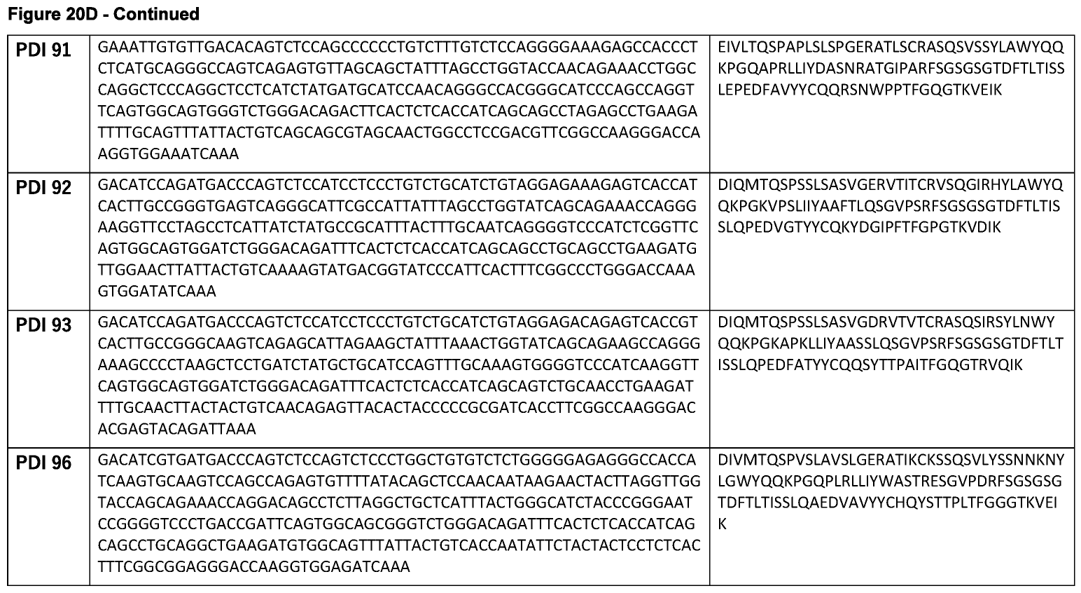

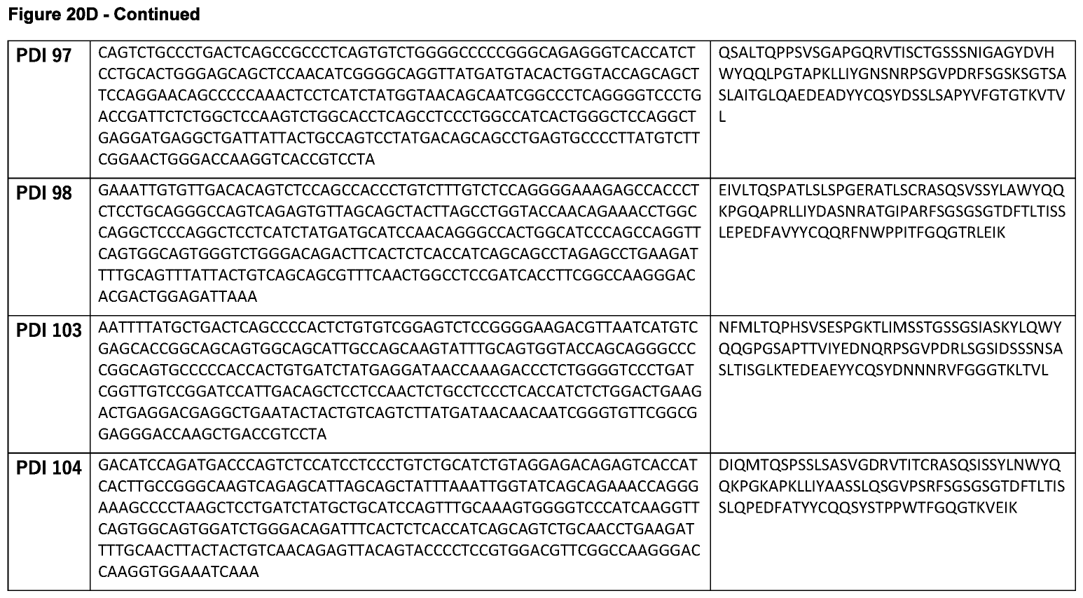

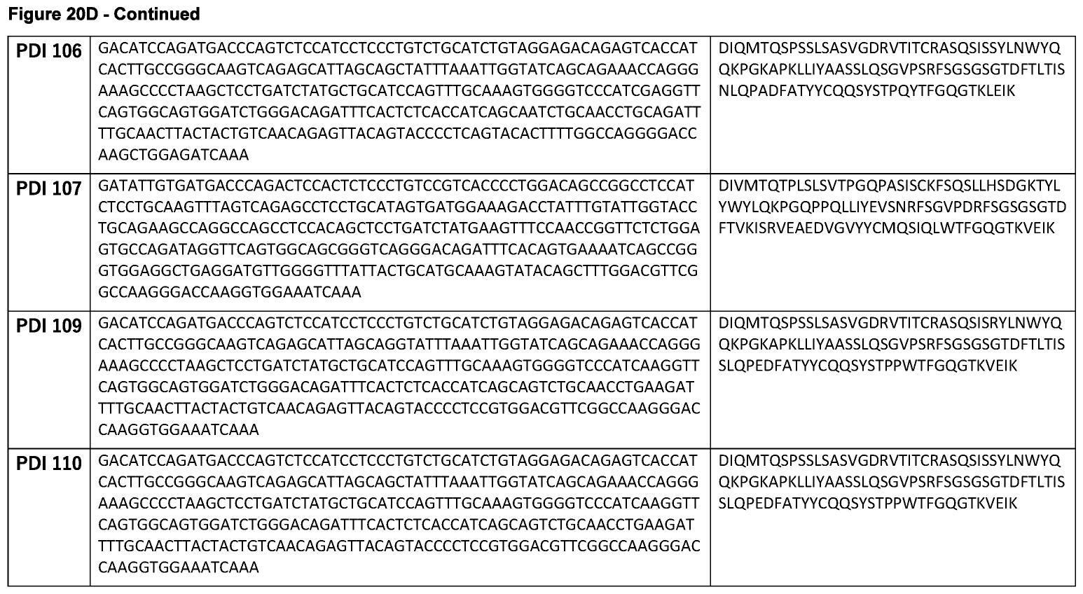

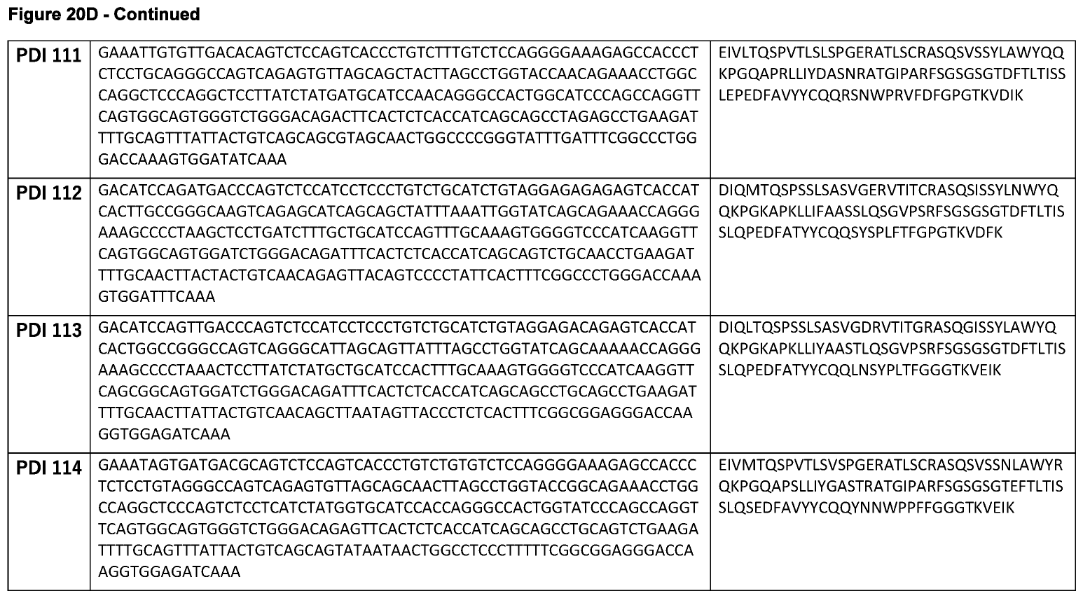

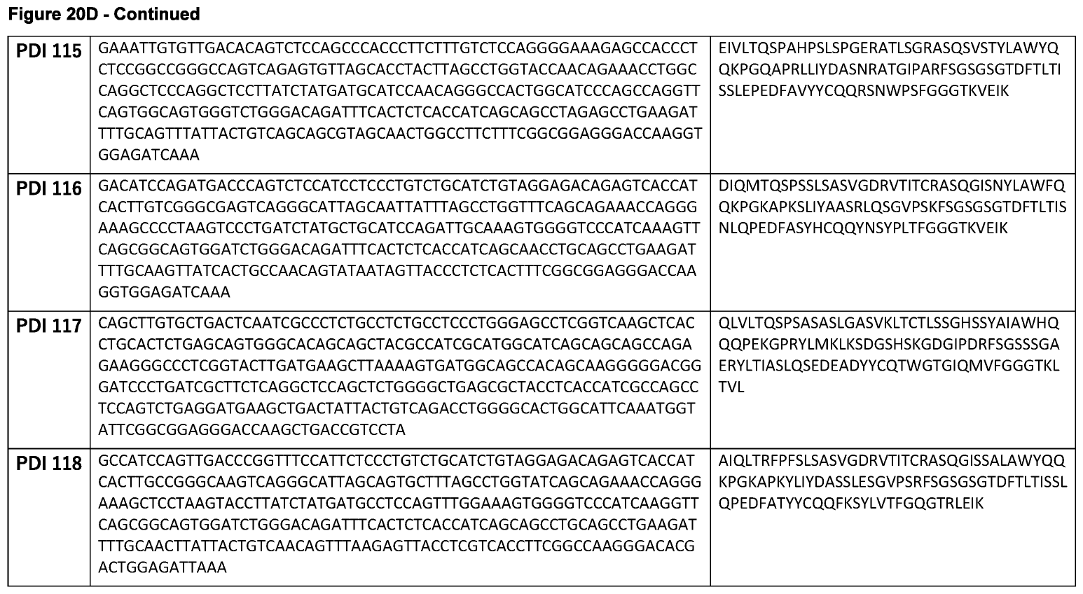

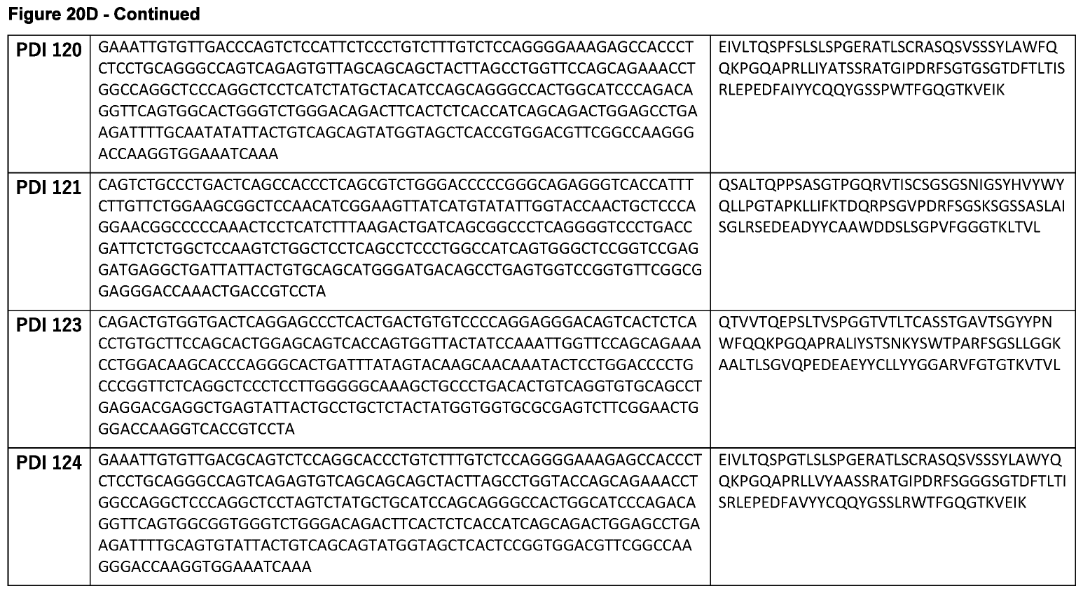

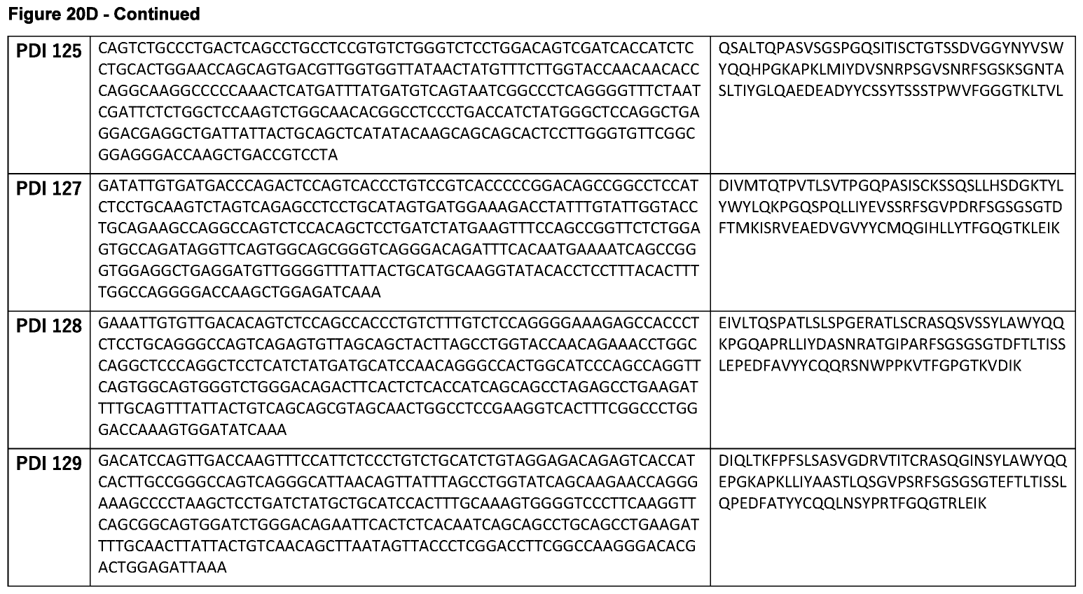

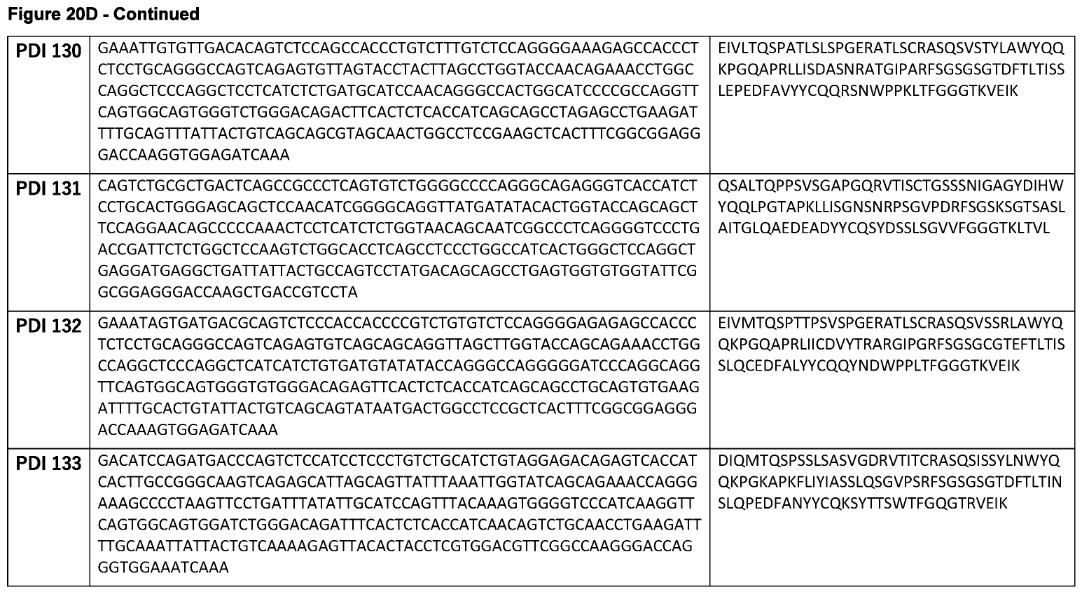

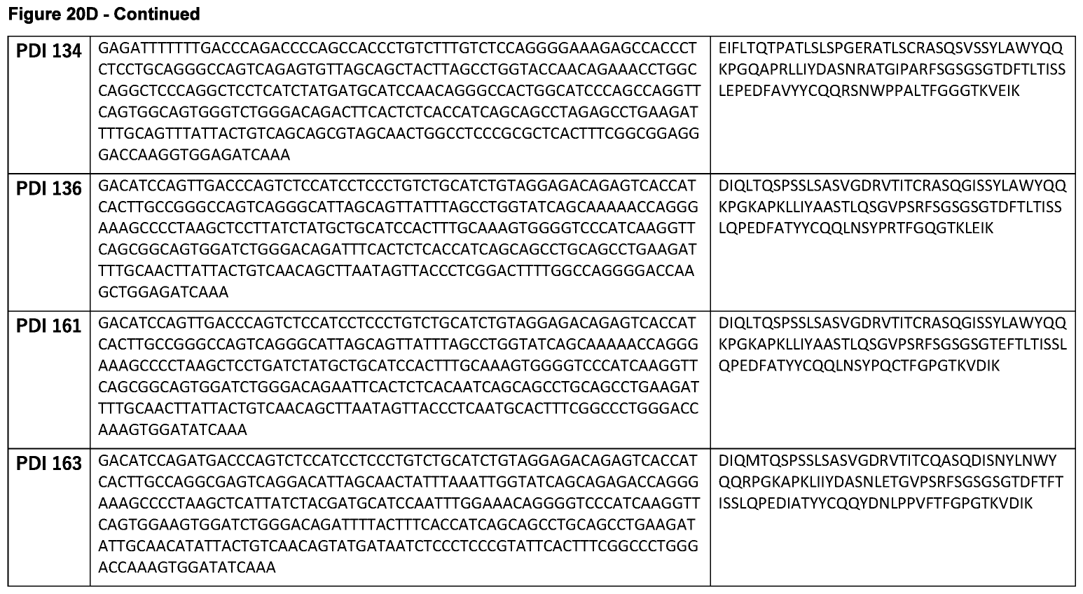

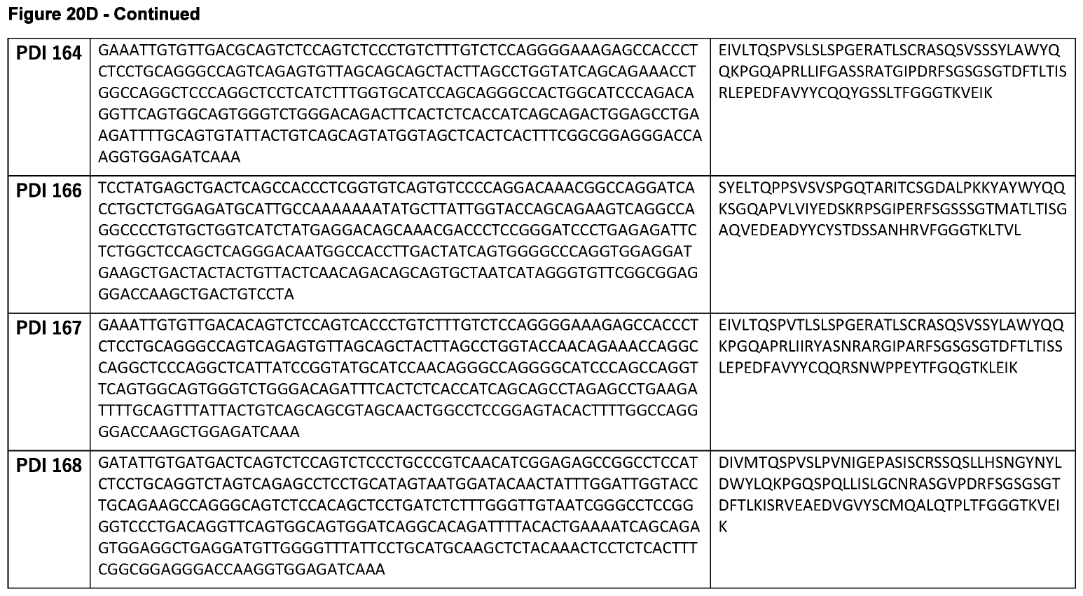

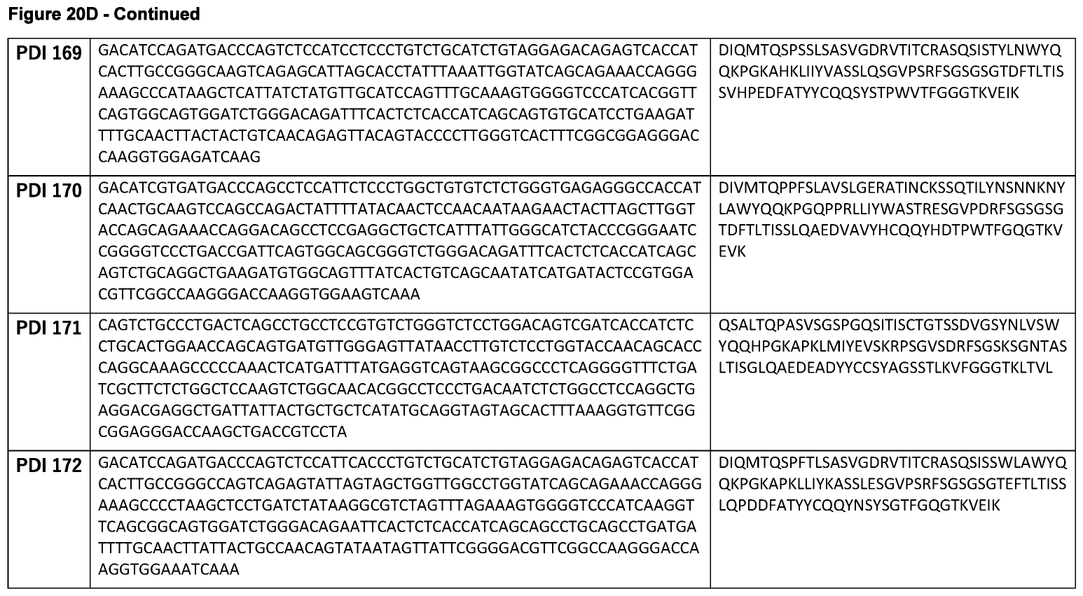

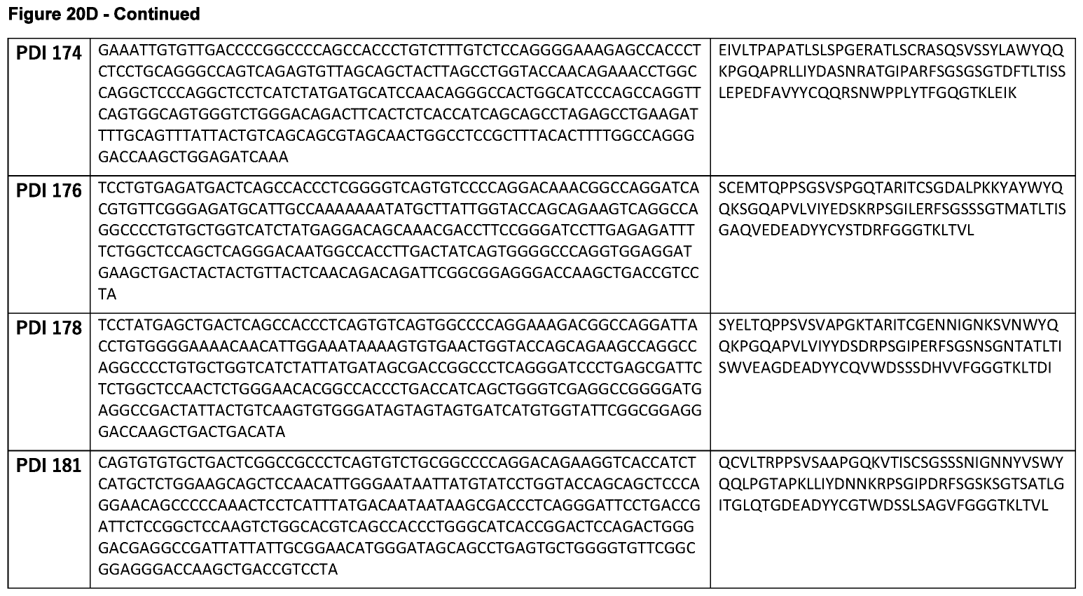

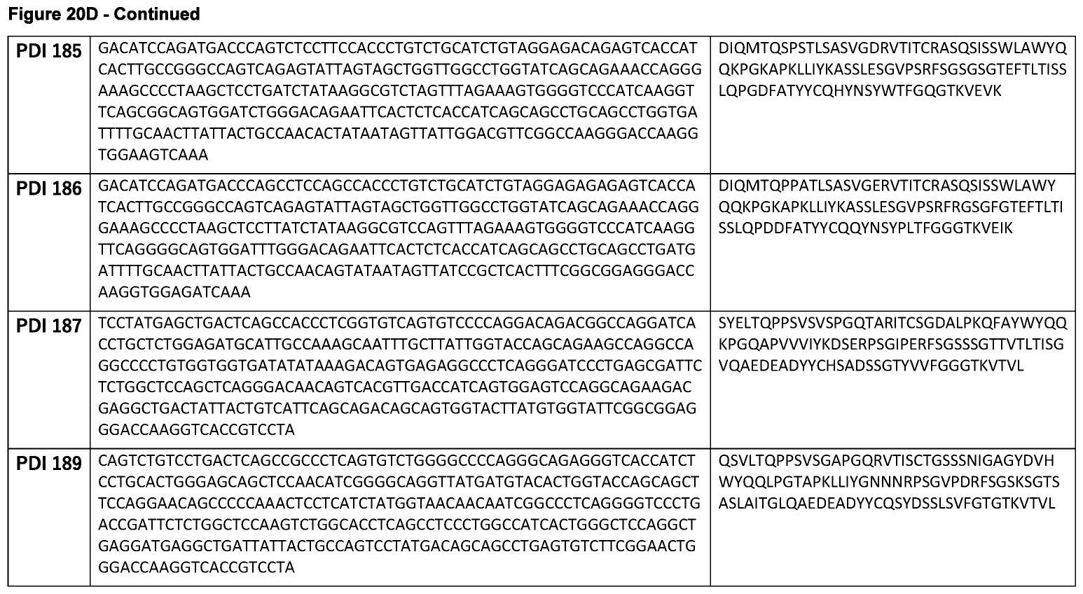

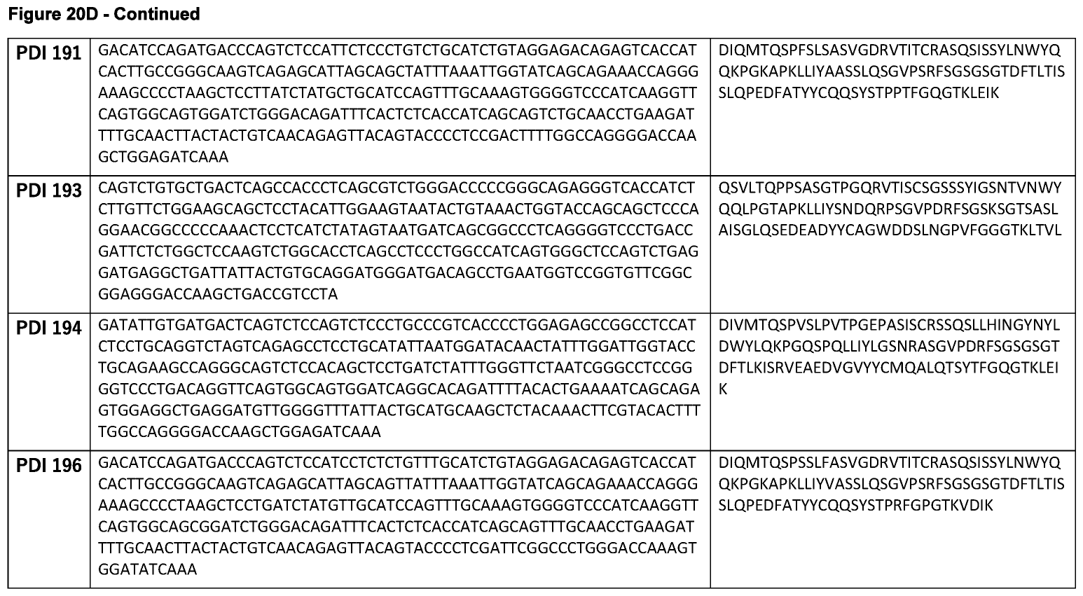

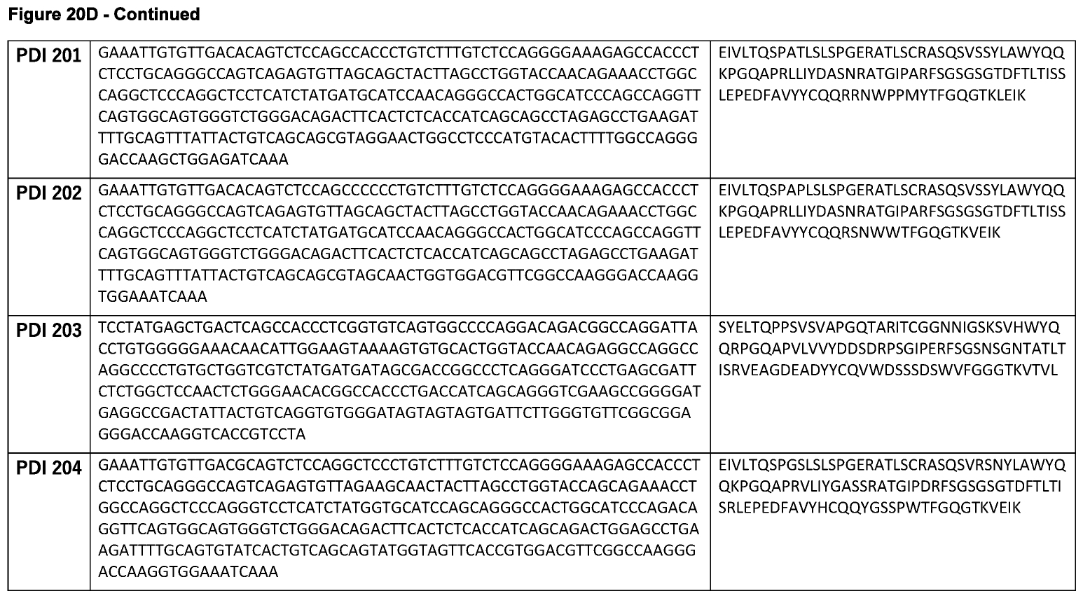

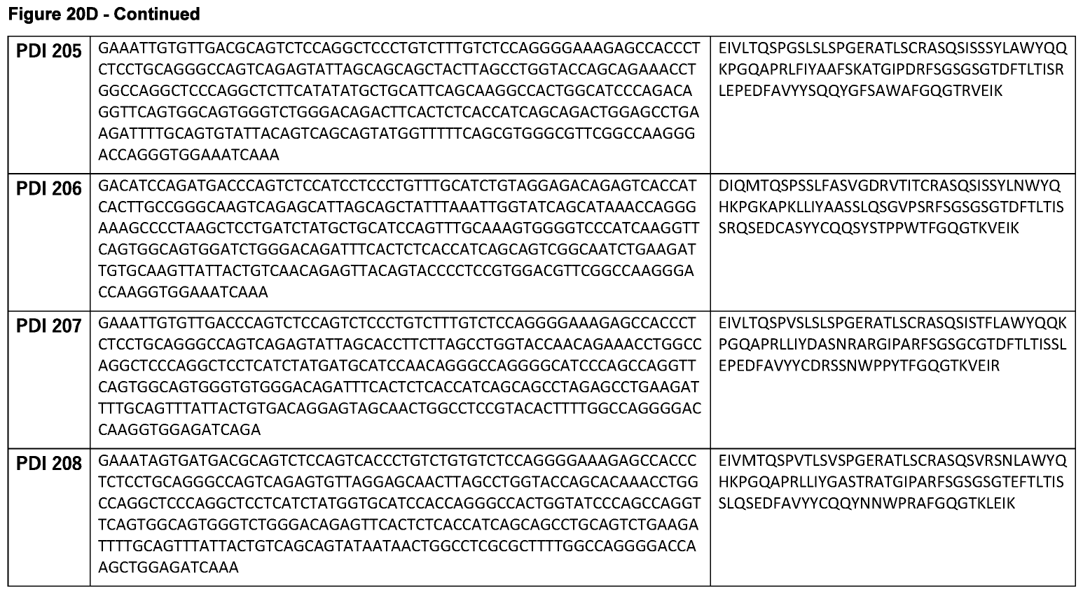

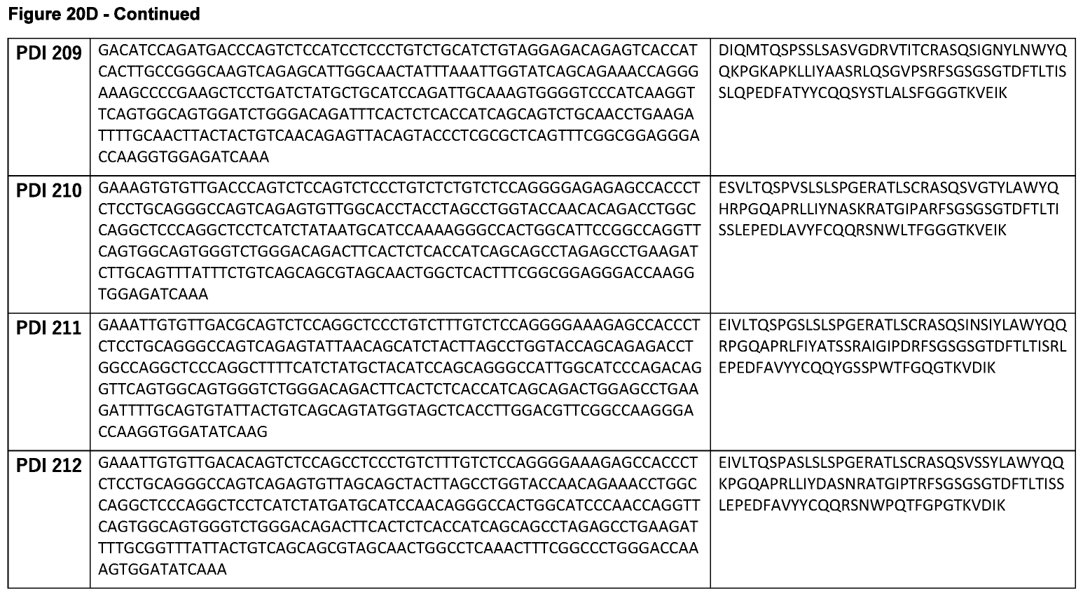

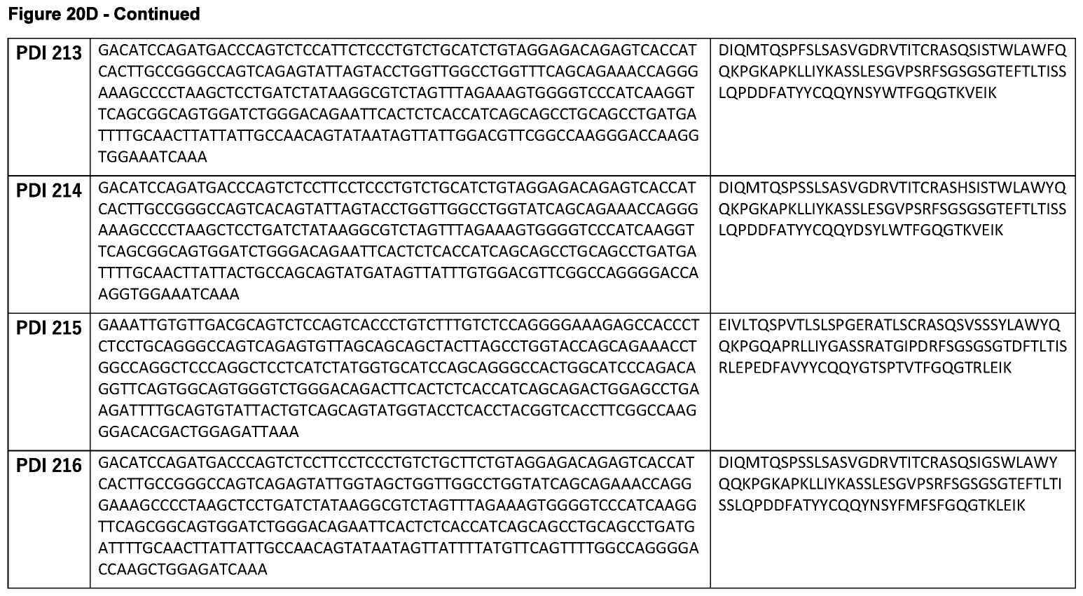

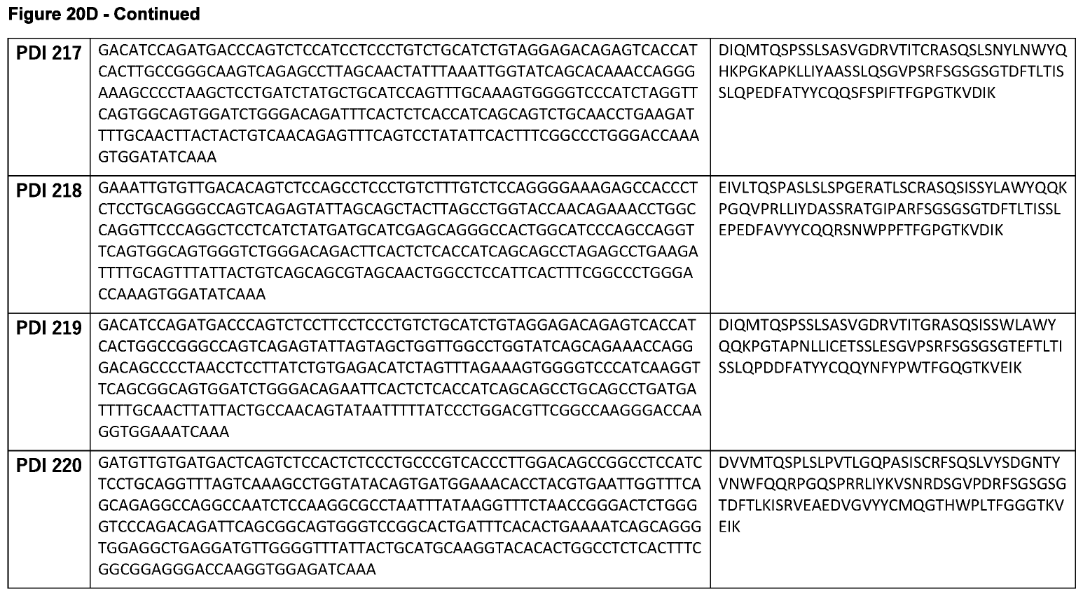

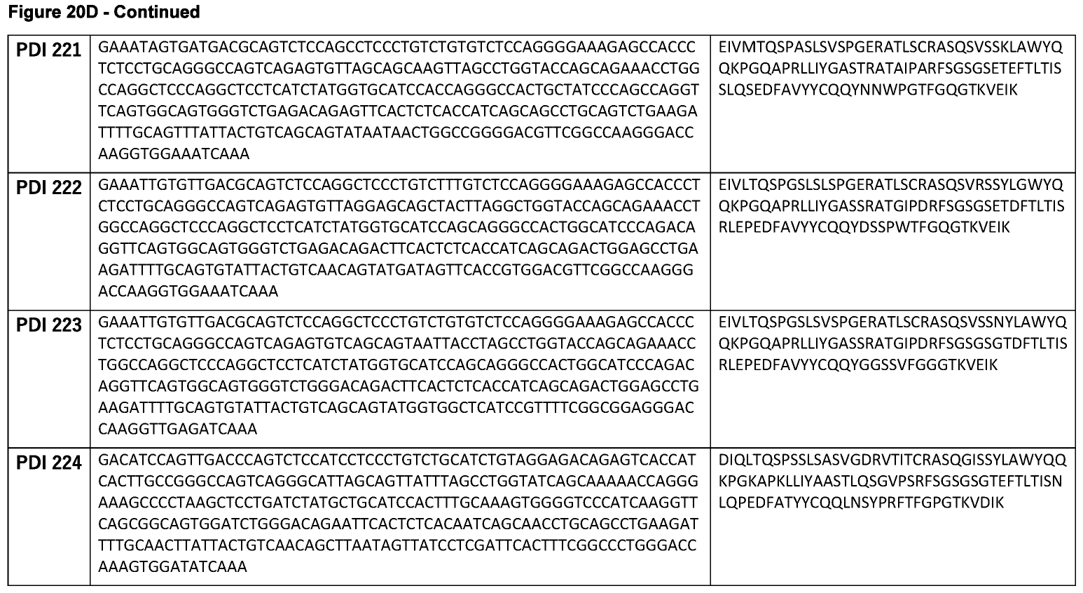

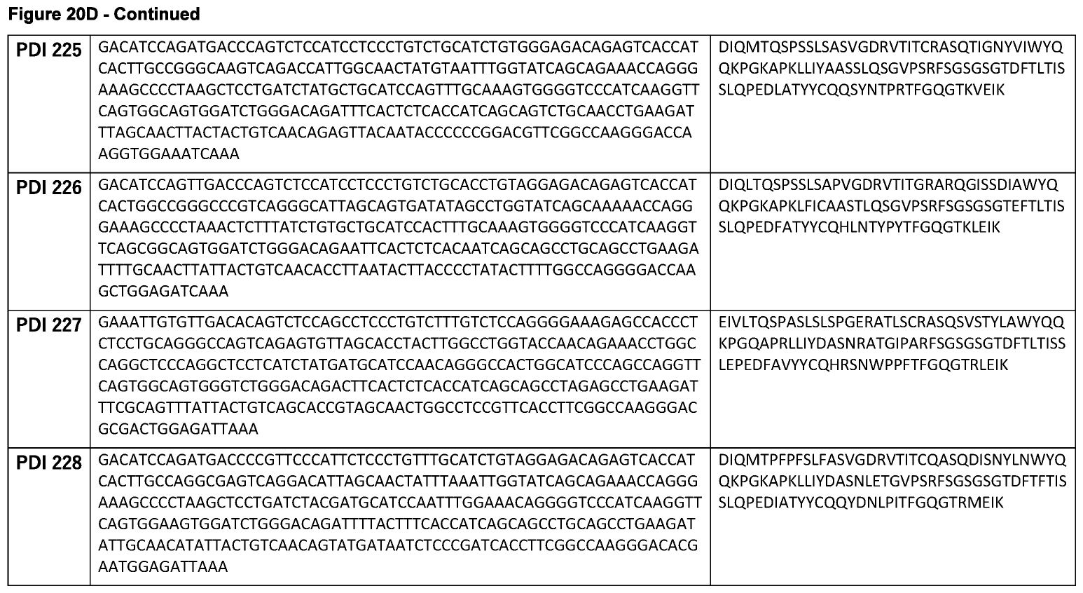

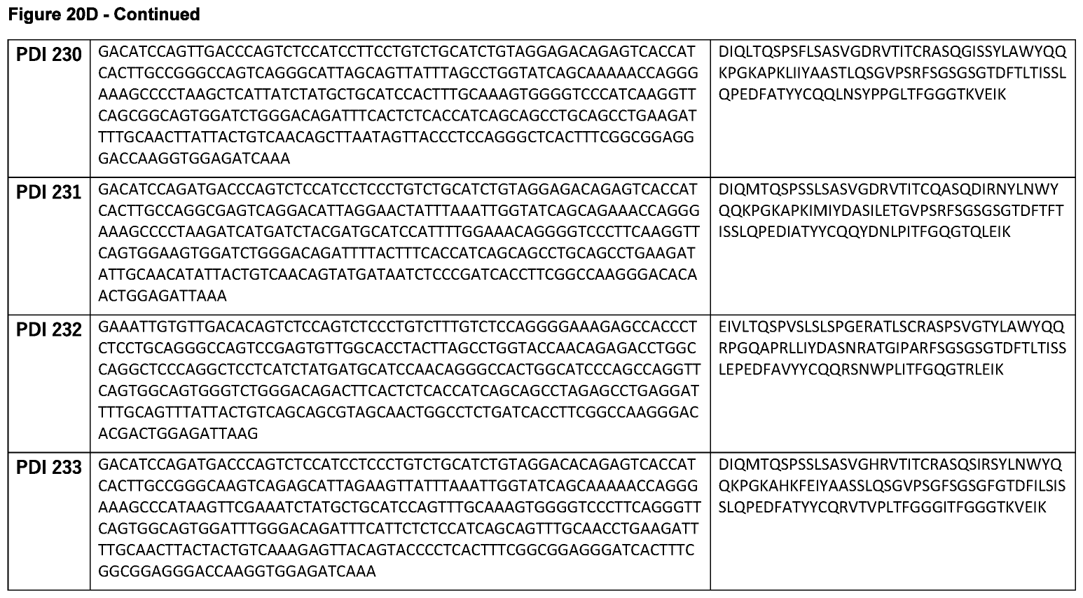

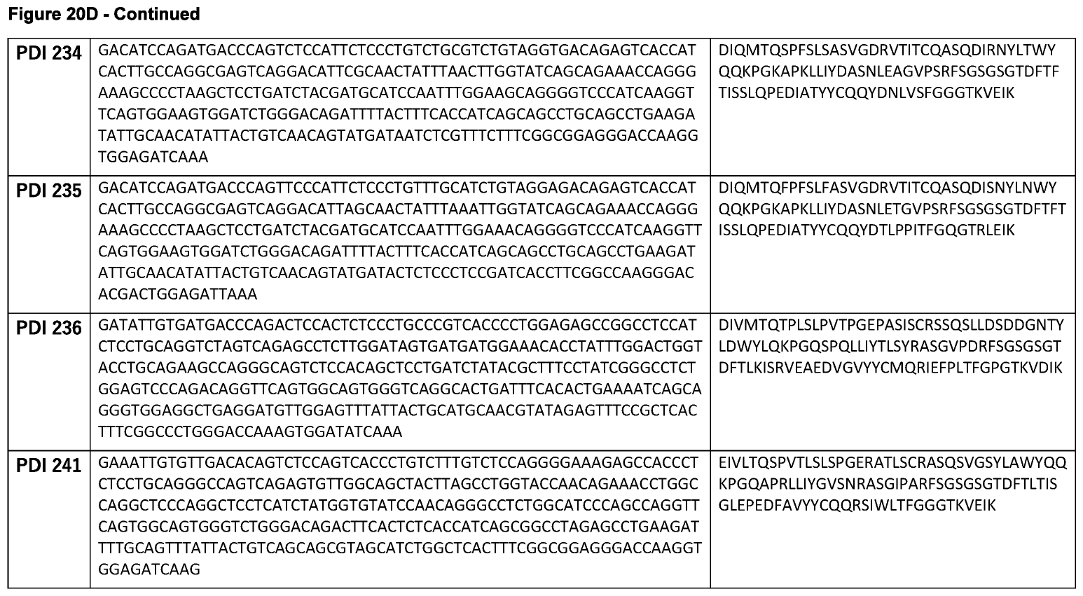

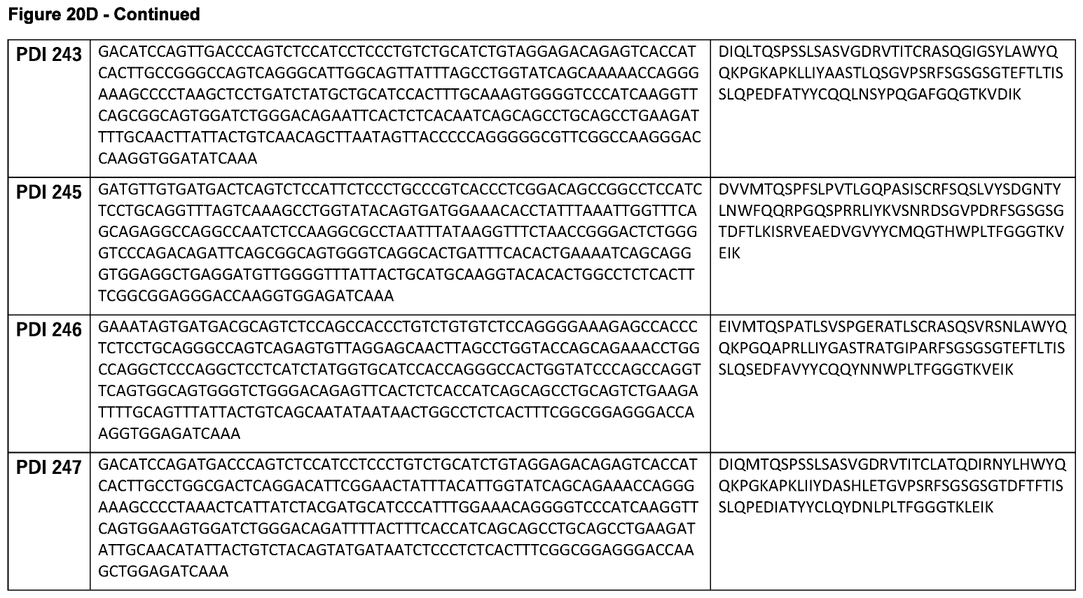

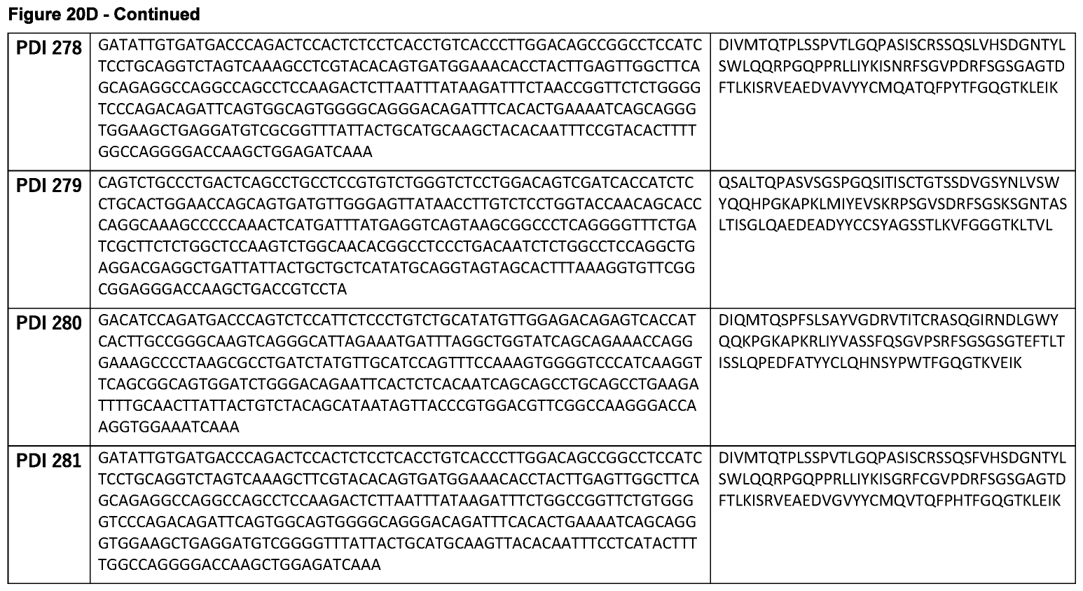

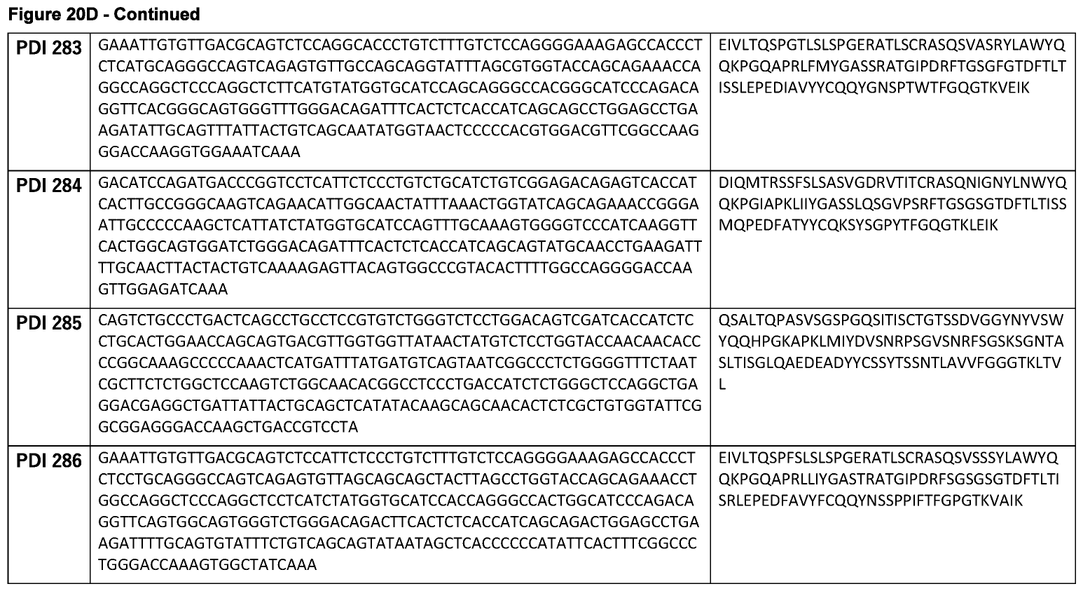

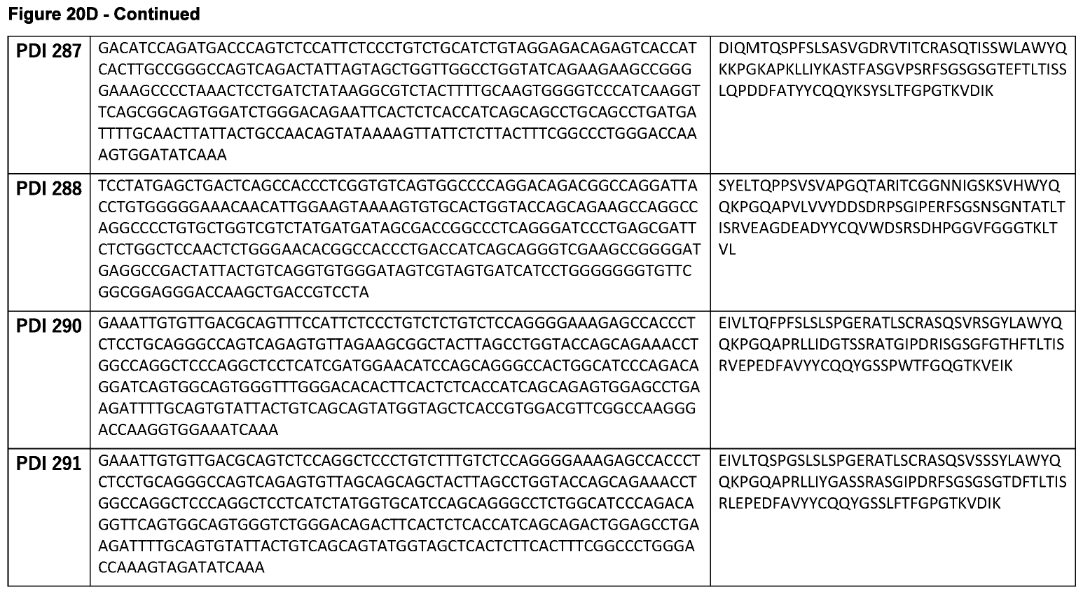

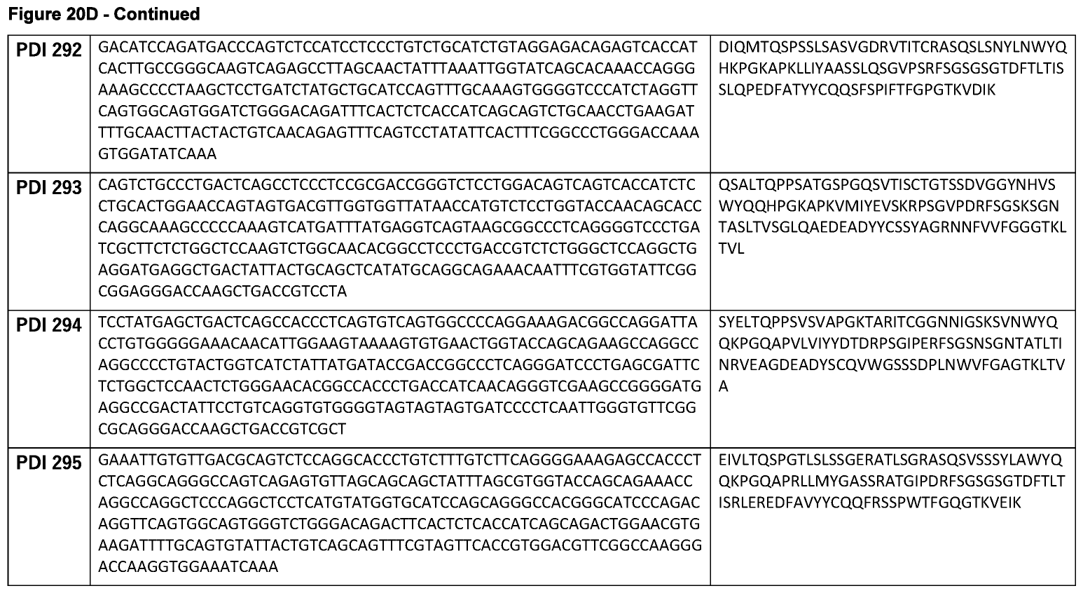

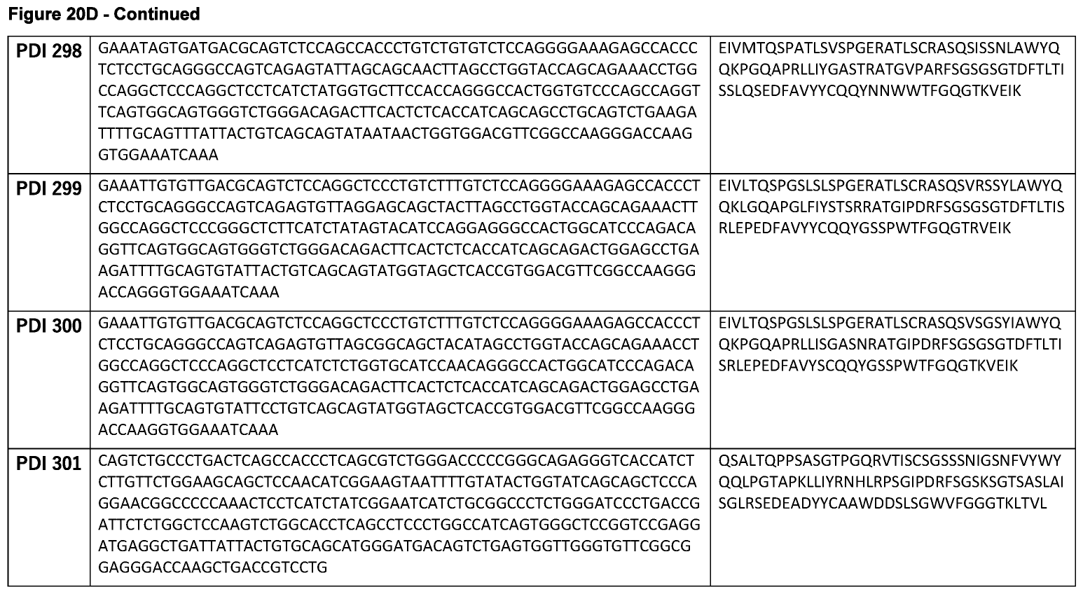

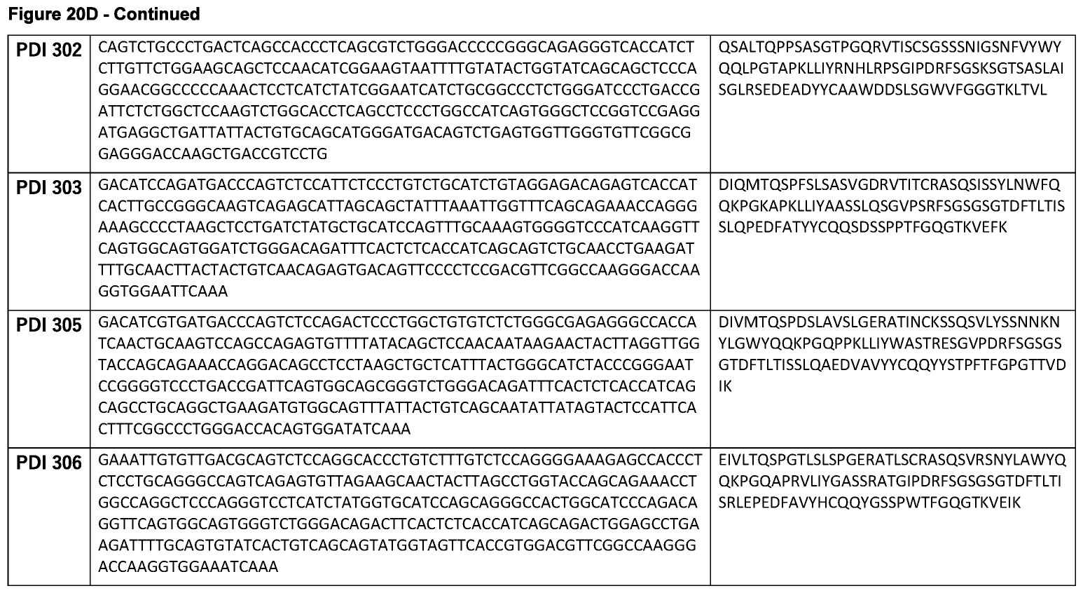

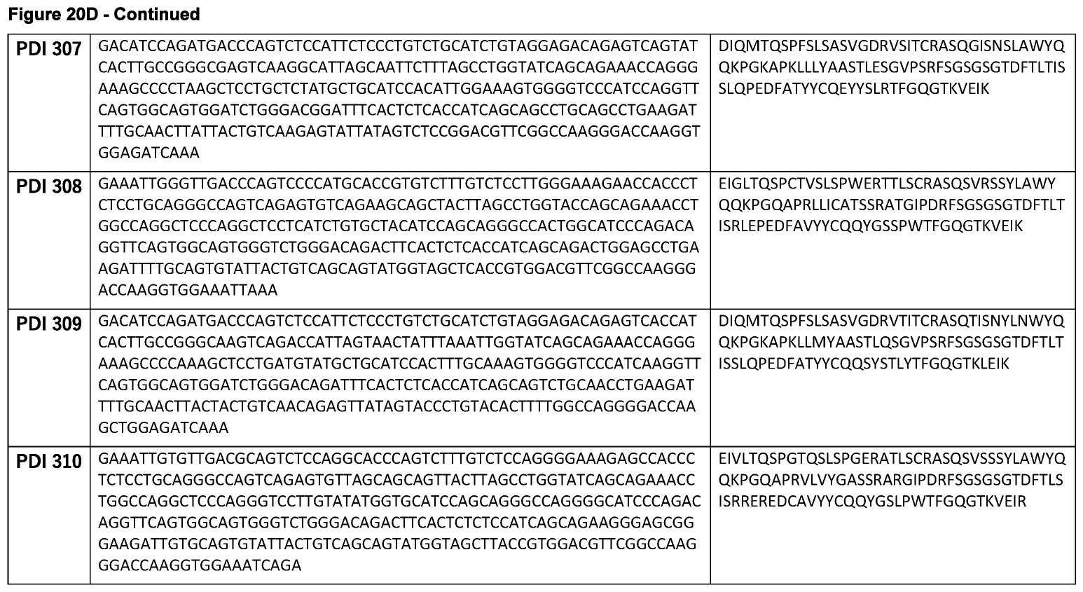

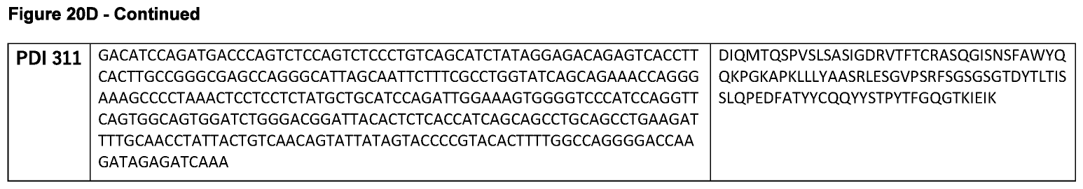

The utility of combining two or more antibodies into cocktails to limit the selection of viral escape mutants has been established in vitro (Baum et al., 2020b; Weisblum et al., 2020). Now in the context of emerging VOC, antibody cocktails also provide a logical strategy for redundant protection in the face of ongoing viral evolution. However, the parameters for optimal cocktail selection remain unclear. Utilising library-panning approaches and targeted isolation from convalescent subjects, the present inventors have characterised a diverse panel of 92 neutralising human mAbs including comprehensive structural analysis of a subset. Highly potent mAbs bound one of up to putative epitope clusters localised across all faces of the RBD, which except for PDI (FIG. 20), efficiently blocked engagement of the cellular receptor ACE2. Single antibodies from a range of different epitope clusters were tested for antiviral effect in vivo using a murine challenge model, with all mAbs tested displaying protection at high dose, for example see Example 5.

The present inventors have demonstrated that the antibodies described herein bind to different epitope clusters, and can be used alone or in combination to neutralise SARS-CoV-2. As discussed herein, the extent of viral suppression correlated with in vitro measurements of neutralising activity but not binding affinity, suggesting functional potency is a key defining metric for protective efficacy

Accordingly, in one embodiment, the present invention provides a composition comprising at least one antibody or an antigen-binding fragment thereof as described herein and a pharmaceutically acceptable carrier.

The term “pharmaceutically acceptable carrier” includes any inert substance that is combined with the at least one antibody or an antigen-binding fragment thereof for preparing an agreeable or convenient dosage form. The “pharmaceutically acceptable carrier” is an excipient that is non-toxic to recipients at the dosages and concentrations employed and is compatible with other ingredients of the formulation comprising at least one antibody or an antigen-binding fragment thereof.

Combinations of two non-competing RBD-specific antibodies have reached late-stage clinical development or approval (Baum et al., 2020a; Jones et al., 2021; Zost et al., 2020a), and strong protective effects following prophylactic administration of analogous cocktails in mice and hamsters was observed in Example 5. The most potent combination was structurally comparable to the Regeneron cocktail and comprised mAb PDI 96 binding a REGN10987-like epitope, and PDI 222, a VH1-58 class antibody binding the upper RBD. The inclusion of a third non-competing antibody (PDI 215), albeit with comparatively lower in vitro neutralisation potency, did not enhance protection in vivo suggesting maintenance of maximal potency is paramount. Without wishing to be bound by theory the present inventors propose there are combinatorial advantages for a three or more mAb cocktail binding diverse epitopes, by allowing for two or more active agents to be maintained in the face of variants of concern and thereby limiting the de novo emergence of escape variants in treatment settings. A dependence for mAb interactions with Fc-receptor (FcR) bearing immune effector cells has been established for maximal protection in vivo for some RBD-specific mAbs in mice (Schafer et al., 2021). However we find no reductions in protective capacity for the PDI 222/PDI 96 cocktail in hamsters. In addition, antibody-FcR engagement has been reported to be greater importance in treatment settings than when used as prophylaxis (Winkler et al., 2021).

In another embodiment, the present invention provides a composition comprising at least two antibodies or an antigen-binding fragments thereof as described herein and a pharmaceutically acceptable carrier.

In another embodiment, the present invention provides a composition comprising at least three antibodies or an antigen-binding fragments thereof as described herein and a pharmaceutically acceptable carrier.

In another embodiment, the present invention provides a composition comprising at least four antibodies or an antigen-binding fragments thereof as described herein and a pharmaceutically acceptable carrier.

In one embodiment, the present invention provides a composition comprising a first antibody or an antigen-binding fragment thereof that binds to a first epitope cluster of the SARS-CoV-2 spike RBD and a second antibody or an antigen-binding fragment thereof that binds to a second epitope cluster of the SARS-CoV-2 spike RBD, and a pharmaceutically acceptable carrier.

In one embodiment, the first epitope cluster is selected from the group consisting of epitope cluster 1, epitope clusters 2, epitope cluster 3, epitope cluster 4, epitope cluster 5 and epitope cluster 6.

In one embodiment, wherein the first epitope cluster is epitope cluster 1, the first antibody or antigen binding fragment thereof is selected from the group consisting of;
an antibody or an antigen-binding fragment thereof wherein the antibody or an antigen-binding fragment thereof comprises a HCDR1 of EFIVSRNY (SEQ ID NO: 18), a HCDR2 of IYSGGTT (SEQ ID NO: 44), a HCDR3 of ARDRGDYLFDY (SEQ ID NO: 70), a LCDR1 of QSISSW (SEQ ID NO: 96), a LCDR2 of KAS (SEQ ID NO: 122), and a LCDR3 of QQYNSYFPT (SEQ ID NO: 148);
an antibody or an antigen-binding fragment thereof wherein the antibody or an antigen-binding fragment thereof comprises a HCDR1 of GLTVSSNY (SEQ ID NO: 20), a HCDR2 of FYPGGST (SEQ ID NO: 46), a HCDR3 of ARDAVYYGMDV (SEQ ID NO: 72), a LCDR1 of QSISSY (SEQ ID NO: 98), a LCDR2 of AAS (SEQ ID NO: 124), and a LCDR3 of QESYSTPGLFT (SEQ ID NO: 150);
an antibody or an antigen-binding fragment thereof wherein the antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134); and
an antibody or an antigen-binding fragment thereof wherein the antibody or an antigen-binding fragment thereof comprises a HCDR1 of GLTVSSNY (SEQ ID NO: 19), a HCDR2 of IYSGGST (SEQ ID NO: 45), a HCDR3 of ARVGGYCSSANCVSDV (SEQ ID NO: 71), a LCDR1 of QDIRNY (SEQ ID NO: 97), a LCDR2 of DAS (SEQ ID NO: 123), and a LCDR3 of QQYDNLPIT (SEQ ID NO: 149);
   and the second antibody epitope cluster is cluster 2, the second antibody or antigen binding fragment thereof comprises a HCDR1 of GETLSGYY (SEQ ID NO: 11), a HCDR2 of ISLRGTA (SEQ ID NO: 37), a HCDR3 of VGGVVLDNVVWFDP (SEQ ID NO: 63), a LCDR1 of QSVGTY (SEQ ID NO: 89), a LCDR2 of NAS (SEQ ID NO: 115), and a LCDR3 of QQRSNWLT (SEQ ID NO: 141).

In another embodiment, wherein the first epitope cluster is epitope cluster 1, the first antibody or antigen binding fragment thereof is selected from the group consisting of;
an antibody or an antigen-binding fragment thereof wherein the antibody or an antigen-binding fragment thereof comprises a HCDR1 of EFIVSRNY (SEQ ID NO: 18), a HCDR2 of IYSGGTT (SEQ ID NO: 44), a HCDR3 of ARDRGDYLFDY (SEQ ID NO: 70), a LCDR1 of QSISSW (SEQ ID NO: 96), a LCDR2 of KAS (SEQ ID NO: 122), and a LCDR3 of QQYNSYFPT (SEQ ID NO: 148);
an antibody or an antigen-binding fragment thereof wherein the antibody or an antigen-binding fragment thereof comprises a HCDR1 of GLTVSSNY (SEQ ID NO: 20), a HCDR2 of FYPGGST (SEQ ID NO: 46), a HCDR3 of ARDAVYYGMDV (SEQ ID NO: 72), a LCDR1 of QSISSY (SEQ ID NO: 98), a LCDR2 of AAS (SEQ ID NO: 124), and a LCDR3 of QESYSTPGLFT (SEQ ID NO: 150);
an antibody or an antigen-binding fragment thereof wherein the antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134); and
an antibody or an antigen-binding fragment thereof wherein the antibody or an antigen-binding fragment thereof comprises a HCDR1 of GLTVSSNY (SEQ ID NO: 19), a HCDR2 of IYSGGST (SEQ ID NO: 45), a HCDR3 of ARVGGYCSSANCVSDV (SEQ ID NO: 71), a LCDR1 of QDIRNY (SEQ ID NO: 97), a LCDR2 of DAS (SEQ ID NO: 123), and a LCDR3 of QQYDNLPIT (SEQ ID NO: 149);
   and the second antibody epitope cluster is cluster 4, the second antibody or antigen binding fragment thereof comprises a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154).

In another embodiment, wherein the first epitope cluster is epitope cluster 1, the first antibody or antigen binding fragment thereof is selected from the group consisting of;
an antibody or an antigen-binding fragment thereof wherein the antibody or an antigen-binding fragment thereof comprises a HCDR1 of EFIVSRNY (SEQ ID NO: 18), a HCDR2 of IYSGGTT (SEQ ID NO: 44), a HCDR3 of ARDRGDYLFDY (SEQ ID NO: 70), a LCDR1 of QSISSW (SEQ ID NO: 96), a LCDR2 of KAS (SEQ ID NO: 122), and a LCDR3 of QQYNSYFPT (SEQ ID NO: 148);
an antibody or an antigen-binding fragment thereof wherein the antibody or an antigen-binding fragment thereof comprises a HCDR1 of GLTVSSNY (SEQ ID NO: 20), a HCDR2 of FYPGGST (SEQ ID NO: 46), a HCDR3 of ARDAVYYGMDV (SEQ ID NO: 72), a LCDR1 of QSISSY (SEQ ID NO: 98), a LCDR2 of AAS (SEQ ID NO: 124), and a LCDR3 of QESYSTPGLFT (SEQ ID NO: 150);
an antibody or an antigen-binding fragment thereof wherein the antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134); and
an antibody or an antigen-binding fragment thereof wherein the antibody or an antigen-binding fragment thereof comprises a HCDR1 of GLTVSSNY (SEQ ID NO: 19), a HCDR2 of IYSGGST (SEQ ID NO: 45), a HCDR3 of ARVGGYCSSANCVSDV (SEQ ID NO: 71), a LCDR1 of QDIRNY (SEQ ID NO: 97), a LCDR2 of DAS (SEQ ID NO: 123), and a LCDR3 of QQYDNLPIT (SEQ ID NO: 149);
   and the second antibody epitope cluster is cluster 4, the second antibody or antigen binding fragment thereof comprises a HCDR1 of GFTFSDYY (SEQ ID NO: 24), a HCDR2 of ISGGSSFT (SEQ ID NO: 50), and a HCDR3 of ARSPPTGDSSDWYDSPAYNNYYMDV (SEQ ID NO: 76).

In a further embodiment, wherein the first epitope cluster is epitope cluster 1, the first antibody or antigen binding fragment thereof is selected from the group consisting of;
an antibody or an antigen-binding fragment thereof wherein the antibody or an antigen-binding fragment thereof comprises a HCDR1 of EFIVSRNY (SEQ ID NO: 18), a HCDR2 of IYSGGTT (SEQ ID NO: 44), a HCDR3 of ARDRGDYLFDY (SEQ ID NO: 70), a LCDR1 of QSISSW (SEQ ID NO: 96), a LCDR2 of KAS (SEQ ID NO: 122), and a LCDR3 of QQYNSYFPT (SEQ ID NO: 148);
an antibody or an antigen-binding fragment thereof wherein the antibody or an antigen-binding fragment thereof comprises a HCDR1 of GLTVSSNY (SEQ ID NO: 20), a HCDR2 of FYPGGST (SEQ ID NO: 46), a HCDR3 of ARDAVYYGMDV (SEQ ID NO: 72), a LCDR1 of QSISSY (SEQ ID NO: 98), a LCDR2 of AAS (SEQ ID NO: 124), and a LCDR3 of QESYSTPGLFT (SEQ ID NO: 150);
an antibody or an antigen-binding fragment thereof wherein the antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134); and
an antibody or an antigen-binding fragment thereof wherein the antibody or an antigen-binding fragment thereof comprises a HCDR1 of GLTVSSNY (SEQ ID NO: 19), a HCDR2 of IYSGGST (SEQ ID NO: 45), a HCDR3 of ARVGGYCSSANCVSDV (SEQ ID NO: 71), a LCDR1 of QDIRNY (SEQ ID NO: 97), a LCDR2 of DAS (SEQ ID NO: 123), and a LCDR3 of QQYDNLPIT (SEQ ID NO: 149);
   and the second antibody epitope cluster is cluster 5, the second antibody or antigen binding fragment thereof comprises a HCDR1 of GFTFSYAW (SEQ ID NO: 21), a HCDR2 of IKRKSDGGTT (SEQ ID NO: 47), a HCDR3 of TTDLCRSTSCEHDAFDI (SEQ ID NO: 73), a LCDR1 of QSIRSY (SEQ ID NO: 99), a LCDR2 of AAS (SEQ ID NO: 125), and a LCDR3 of QQSYTTPAIT (SEQ ID NO: 151).

In a further embodiment, wherein the first epitope cluster is epitope cluster 1, the first antibody or antigen binding fragment thereof is selected from the group consisting of;
an antibody or an antigen-binding fragment thereof wherein the antibody or an antigen-binding fragment thereof comprises a HCDR1 of EFIVSRNY (SEQ ID NO: 18), a HCDR2 of IYSGGTT (SEQ ID NO: 44), a HCDR3 of ARDRGDYLFDY (SEQ ID NO: 70), a LCDR1 of QSISSW (SEQ ID NO: 96), a LCDR2 of KAS (SEQ ID NO: 122), and a LCDR3 of QQYNSYFPT (SEQ ID NO: 148);
an antibody or an antigen-binding fragment thereof wherein the antibody or an antigen-binding fragment thereof comprises a HCDR1 of GLTVSSNY (SEQ ID NO: 20), a HCDR2 of FYPGGST (SEQ ID NO: 46), a HCDR3 of ARDAVYYGMDV (SEQ ID NO: 72), a LCDR1 of QSISSY (SEQ ID NO: 98), a LCDR2 of AAS (SEQ ID NO: 124), and a LCDR3 of QESYSTPGLFT (SEQ ID NO: 150);
an antibody or an antigen-binding fragment thereof wherein the antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134); and
an antibody or an antigen-binding fragment thereof wherein the antibody or an antigen-binding fragment thereof comprises a HCDR1 of GLTVSSNY (SEQ ID NO: 19), a HCDR2 of IYSGGST (SEQ ID NO: 45), a HCDR3 of ARVGGYCSSANCVSDV (SEQ ID NO: 71), a LCDR1 of QDIRNY (SEQ ID NO: 97), a LCDR2 of DAS (SEQ ID NO: 123), and a LCDR3 of QQYDNLPIT (SEQ ID NO: 149);
   and the second antibody epitope cluster is cluster 6, the second antibody or antigen binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133).

In a further embodiment, wherein the first epitope cluster is epitope cluster 2, the first antibody or antigen binding fragment thereof comprises a HCDR1 of GETLSGYY (SEQ ID NO: 11), a HCDR2 of ISLRGTA (SEQ ID NO: 37), a HCDR3 of VGGVVLDNVVWFDP (SEQ ID NO: 63), a LCDR1 of QSVGTY (SEQ ID NO: 89), a LCDR2 of NAS (SEQ ID NO: 115), and a LCDR3 of QQRSNWLT (SEQ ID NO: 141), and the second antibody epitope cluster is cluster 4, the second antibody or antigen binding fragment thereof comprises a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154).

In a further embodiment, wherein the first epitope cluster is epitope cluster 2, the first antibody or antigen binding fragment thereof comprises a HCDR1 of GETLSGYY (SEQ ID NO: 11), a HCDR2 of ISLRGTA (SEQ ID NO: 37), a HCDR3 of VGGVVLDNVVWFDP (SEQ ID NO: 63), a LCDR1 of QSVGTY (SEQ ID NO: 89), a LCDR2 of NAS (SEQ ID NO: 115), and a LCDR3 of QQRSNWLT (SEQ ID NO: 141), and the second antibody epitope cluster is cluster 4, the second antibody or antigen binding fragment thereof comprises a HCDR1 of GFTFSDYY (SEQ ID NO: 24), a HCDR2 of ISGGSSFT (SEQ ID NO: 50), and a HCDR3 of ARSPPTGDSSDWYDSPAYNNYYMDV (SEQ ID NO: 76)

In a further embodiment, wherein the first epitope cluster is epitope cluster 2, the first antibody or antigen binding fragment thereof comprises a HCDR1 of GETLSGYY (SEQ ID NO: 11), a HCDR2 of ISLRGTA (SEQ ID NO: 37), a HCDR3 of VGGVVLDNVVWFDP (SEQ ID NO: 63), a LCDR1 of QSVGTY (SEQ ID NO: 89), a LCDR2 of NAS (SEQ ID NO: 115), and a LCDR3 of QQRSNWLT (SEQ ID NO: 141), and the second antibody epitope cluster is cluster 5, the second antibody or antigen binding fragment thereof comprises a HCDR1 of GFTFSYAW (SEQ ID NO: 21), a HCDR2 of IKRKSDGGTT (SEQ ID NO: 47), a HCDR3 of TTDLCRSTSCEHDAFDI (SEQ ID NO: 73), a LCDR1 of QSIRSY (SEQ ID NO: 99), a LCDR2 of AAS (SEQ ID NO: 125), and a LCDR3 of QQSYTTPAIT (SEQ ID NO: 151).

In a further embodiment, wherein the first epitope cluster is epitope cluster 2, the first antibody or antigen binding fragment thereof comprises a HCDR1 of GETLSGYY (SEQ ID NO: 11), a HCDR2 of ISLRGTA (SEQ ID NO: 37), a HCDR3 of VGGVVLDNVVWFDP (SEQ ID NO: 63), a LCDR1 of QSVGTY (SEQ ID NO: 89), a LCDR2 of NAS (SEQ ID NO: 115), and a LCDR3 of QQRSNWLT (SEQ ID NO: 141), and the second antibody epitope cluster is cluster 6, the second antibody or antigen binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133).

In a further embodiment, wherein the first epitope cluster is epitope cluster 4, the first antibody or antigen binding fragment thereof comprises a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154), and the second antibody epitope cluster is cluster 5, the second antibody or antigen binding fragment thereof comprises a HCDR1 of GFTFSYAW (SEQ ID NO: 21), a HCDR2 of IKRKSDGGTT (SEQ ID NO: 47), a HCDR3 of TTDLCRSTSCEHDAFDI (SEQ ID NO: 73), a LCDR1 of QSIRSY (SEQ ID NO: 99), a LCDR2 of AAS (SEQ ID NO: 125), and a LCDR3 of QQSYTTPAIT (SEQ ID NO: 151).

In a further embodiment, wherein the first epitope cluster is epitope cluster 4, the first antibody or antigen binding fragment thereof comprises a HCDR1 of GFTFSDYY (SEQ ID NO: 24), a HCDR2 of ISGGSSFT (SEQ ID NO: 50), and a HCDR3 of ARSPPTGDSSDWYDSPAYNNYYMDV (SEQ ID NO: 76), and the second antibody epitope cluster is cluster 5, the second antibody or antigen binding fragment thereof comprises a HCDR1 of GFTFSYAW (SEQ ID NO: 21), a HCDR2 of IKRKSDGGTT (SEQ ID NO: 47), a HCDR3 of TTDLCRSTSCEHDAFDI (SEQ ID NO: 73), a LCDR1 of QSIRSY (SEQ ID NO: 99), a LCDR2 of AAS (SEQ ID NO: 125), and a LCDR3 of QQSYTTPAIT (SEQ ID NO: 151).

In a further embodiment, wherein the first epitope cluster is epitope cluster 4, the first antibody or antigen binding fragment thereof comprises a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154), and the second antibody epitope cluster is cluster 6, the second antibody or antigen binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133).

In a further embodiment, wherein the first epitope cluster is epitope cluster 4, the first antibody or antigen binding fragment thereof comprises a HCDR1 of GFTFSDYY (SEQ ID NO: 24), a HCDR2 of ISGGSSFT (SEQ ID NO: 50), and a HCDR3 of ARSPPTGDSSDWYDSPAYNNYYMDV (SEQ ID NO: 76), and the second antibody epitope cluster is cluster 6, the second antibody or antigen binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133).

In a further embodiment, wherein the first epitope cluster is epitope cluster 5, the first antibody or antigen binding fragment thereof comprises a HCDR1 of GFTFSYAW (SEQ ID NO: 21), a HCDR2 of IKRKSDGGTT (SEQ ID NO: 47), a HCDR3 of TTDLCRSTSCEHDAFDI (SEQ ID NO: 73), a LCDR1 of QSIRSY (SEQ ID NO: 99), a LCDR2 of AAS (SEQ ID NO: 125), and a LCDR3 of QQSYTTPAIT (SEQ ID NO: 151), and the second antibody epitope cluster is cluster 6, the second antibody or antigen binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133).

In a preferred embodiment, the present invention provides a composition comprising at least two antibodies or an antigen-binding fragments thereof and a pharmaceutically acceptable carrier, wherein the first antibody or an antigen binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133), and the second antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134).

In a preferred embodiment, the present invention provides a composition comprising at least three antibodies or an antigen-binding fragments thereof and a pharmaceutically acceptable carrier, wherein the first antibody or antigen binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133), and the second antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134), and the third antibody or an antigen binding fragment thereof comprises a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154).

As used herein the term “epitope cluster” refers to the amino acid residues of an antigen (e.g. the SARS-CoV-2 spike RBD) that are contacted by an antibody that binds to that antigen. Epitope clusters that do not overlap with one another are also referred to herein as “non-competing” (for antibody binding) epitopes.

In one embodiment, the first epitope cluster of the SARS-CoV-2 spike RBD and the second epitope cluster of the SARS-CoV-2 spike RBD do not overlap.

In another embodiment, the present invention provides a composition comprising a first antibody or an antigen-binding fragment thereof that binds to a epitope cluster 4 of the SARS-CoV-2 spike RBD and a second antibody or an antigen-binding fragment thereof that binds to an epitope cluster of the SARS-CoV-2 spike RBD overlapping with the ACE2 binding site of the spike protein, and a pharmaceutically acceptable carrier.

In one embodiment, the antibody or an antigen-binding fragment thereof that binds to an epitope cluster 4 of the SARS-CoV-2 spike RBD.

In one embodiment, the antibody or an antigen-binding fragment thereof that binds to an epitope cluster 4 of the SARS-CoV-2 spike RBD comprises a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154).

In another embodiment, the antibody or an antigen-binding fragment thereof that binds to an epitope cluster 4 of the SARS-CoV-2 spike RBD comprises a HCDR1 of GFTFSDYY (SEQ ID NO: 24), a HCDR2 of ISGGSSFT (SEQ ID NO: 50), and a HCDR3 of ARSPPTGDSSDWYDSPAYNNYYMDV (SEQ ID NO: 76).

With regard to the epitope cluster scheme used in Hastie et al. (2021) Science 374(6566): 472-478, “cluster 1” as used herein overlaps with epitope “RBD2b.1”, “cluster 2” as used herein overlaps with epitope “RBD2a”, “cluster 4” as used herein overlaps with epitope “RBD4” or “RBD5”, “cluster 5” as used herein overlaps with epitope “RBD5”, and “cluster 6” as used herein overlaps with epitope “RBD5”.

With regard to the epitope cluster scheme used in Barnes, et al. (2020) Nature 588, 682-687, “cluster 1” as used herein overlaps with epitope “class 1”, “cluster 4” as used herein overlaps with epitope “Class 3”, “cluster 5” as used herein overlaps with epitope “Class 3”, and “cluster 6” as used herein overlaps with epitope “Class 3”.

In one embodiment, the present invention provides a composition comprising a first antibody or an antigen-binding fragment thereof that binds to a first epitope cluster of the SARS-CoV-2 spike RBD and a second antibody or an antigen-binding fragment thereof that binds to the same epitope cluster of the SARS-CoV-2 spike RBD, and a pharmaceutically acceptable carrier.

Without wishing to be bound by theory, the present inventors propose that the use of more than one antibody, including antibodies that bind to different epitope clusters, can be used to treat subjects and reduce the possibility of escape mutations.

Exemplary mutations include substitutions in spike proteins such as those of B.1.1.7 (Alpha): 69del, 70del, 144del, (E484K*), (S494P*), N501Y, A570D, D614G, P681H, T7161, S982A, D1118H (K1191N*), B.1.351 (Beta): D80A, D215G, 241del, 242del, 243del, K417N, E484K, N501Y, D614G, A701V, B.1.617.2 (Delta): T19R, (V70F*), T951, G142D, E156−, F157−, R158G, (A222V*), (W258L*), (K417N*), L452R, T478K, D614G, P681R, D950N, P1 (Gamma): L18F, T20N, P26S, D138Y, R190S, K417T, E484K, N501Y, D614G, H655Y, T10271, B.1.1.529 (Omicron BA.1): G339D, S371L, S373P, S375F, K417N, N440K, G446S, S477N, T478K, E484A, Q493R, G496S, Q498R, N501Y, Y505H, and B.1.1.529 (Omicron BA.2): G339D, S371F, S373P, S375F, T376A, D405N, R408S, K417N, N440K, S477N, T478K, E484A, Q493R, Q498R, N501Y, Y505H. B.1.1.529 (Omicron BA.3): which has the following Spike Protein Substitutions: G339D, S371 F, S373P, S375F, D405N, K417N, N440K, G446S, S477N, T478K, E484A, Q493R, Q498R, N501Y, Y505H, B.1.1.529 (Omicron BA.4): G339D, S371 F, S373P, S375F, T376A, D405N, R408S, K417N, N440K, L452R, S477N, T478K, E484A, F486V; R493Q, Q498R, N501Y, Y505H, B.1.1.529 (Omicron BA.5): G339D, S371 F, S373P, S375F, T376A, D405N, R408S, K417N, N440K, L452R, S477N, T478K, E484A, F486V; R493Q, Q498R, N501Y, Y505H, (Omicron BA.2.75): G339H, S371F, S373P, S375F, T376A, D405N, R408S, K417N, N440K, G446S, N460K, S477N, T478K, E484A, Q493, Q498R, N501Y, Y505H.

Epitopes distal to the critical changes in the RBD at E484 and K417 carried by both B1.351 and P1 VOC, such as those bound by REGN10987-like antibodies (C135 (Barnes et al., 2020b), and PDI 96 described herein), show consistent recognition and neutralisation of VOC. However, mutagenesis suggests that substitutions at G446, while not widely prevalent in circulating virus strains, could potentially drive escape from such mAbs. Importantly, the stereotypic VH1-58 class of mAbs appears resistant to E484 and K417 changes in the RBD. Structurally, this class of antibodies, while binding proximally to both the K417 and E484 RBD residues, do not form contacts with either sidechain and therefore maintain inhibition of ACE2 binding to current SARS-CoV-2 VOC. Similarly, structure-based prediction suggests such mAbs would also be resistant in the face of L452 mutations as carried by fast emerging B.1.617 variants.

The exceptional and conserved potency for the VH1-58 class of mAbs described herein (e.g. (PDI 222, PDI 209, PDI 204) also observed in reported mAbs (Dejnirattisai et al., 2021; Robbiani et al., 2020; Zost et al., 2020a) and found in multiple donors as described herein, suggests this common epitope abridging the ACE2 interaction site is a durable target for broad immunity in the face of viral variants. Elicitation of analogous responses by vaccination, in conjunction with REGN10987-like specificities, is a major consideration for engineering spike immunogens for robust protection in the face of ongoing viral evolution.

In one embodiment the present invention provides an isolated polynucleotide comprising a nucleic acid which encodes an antibody or an antigen-binding fragment thereof molecule as described herein.

As used herein the term “nucleic acid molecule” as used in the present invention refers to a polymeric form of nucleotides and includes both sense and antisense strands of RNA, cDNA, genomic DNA, and synthetic forms and mixed polymers of the above. A nucleotide refers to a ribonucleotide, deoxynucleotide or a modified form of either type of nucleotide. The term also includes single- and double-stranded forms of DNA. In addition, a polynucleotide may include either or both naturally-occurring and modified nucleotides linked together by naturally-occurring and/or non-naturally occurring nucleotide linkages. The nucleic acid molecules may be modified chemically or biochemically or may contain non-natural or derivatized nucleotide bases, as will be readily appreciated by those of skill in the art. A reference to a nucleic acid sequence encompasses its complement unless otherwise specified. Thus, a reference to a nucleic acid molecule having a particular sequence should be understood to encompass its complementary strand, with its complementary sequence.

In another embodiment, the present invention provides a vector comprising the polynucleotide as described herein.

As used herein the term “vector” includes a nucleic acid molecule into which a second nucleic acid molecule can be inserted for introduction into a host where it will be replicated, and in some cases expressed. In other words, a vector is capable of transporting a nucleic acid molecule to which it has been linked. Cloning as well as expression vectors are contemplated by the term “vector”, as used herein. Vectors include, but are not limited to, plasmids, cosmids, bacterial artificial chromosomes (BAC) and yeast artificial chromosomes (YAC) and vectors derived from bacteriophages or plant or animal (including human) viruses. Vectors comprise an origin of replication recognised by the proposed host and in case of expression vectors, promoter and other regulatory regions recognised by the host. A vector containing a second nucleic acid molecule is introduced into a cell by transformation, transfection, or by making use of viral entry mechanisms. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome.

In one embodiment the present invention provides a host cell comprising a polynucleotide as described herein.

In one embodiment the present invention provides a method for prophylaxis or treatment of SARS-CoV-2 infection in a subject comprising administering an effective amount of an antibody or an antigen-binding fragment thereof as described herein.

In one embodiment the present invention provides a method for prophylaxis or treatment of SARS-CoV-2 infection in a subject comprising administering an effective amount of a composition as described herein.

In one embodiment the present invention provides a use of an antibody or an antigen-binding fragment thereof as described herein in the manufacture of a medicament for prophylaxis or treatment of SARS-CoV-2 infection in a subject.

In one embodiment the present invention provides a use of a composition as described herein in the manufacture of a medicament for prophylaxis or treatment of SARS-CoV-2 infection in a subject.

### EXAMPLES

### Example 1: Materials and Methods

### Subject Recruitment and Sample Collection

Individuals recovered from COVID-19 were recruited through contacts with the investigators and invited to provide a blood sample. Whole blood was obtained with sodium heparin anticoagulant. Plasma was collected and stored at −80° C., and PBMCs were isolated via Ficoll-Paque separation, cryopreserved in 10% DMSO/FCS and stored in liquid nitrogen. Selection of the 6 subjects for isolation and sequencing of spike-specific B cells was based upon prior screening for potent plasma neutralising activity in a limiting dilution microneutralisation assay.

### Generation of Recombinant Proteins

The generation of SARS-CoV-2 RBD, hACE2, HKU-1 spike and SARS-CoV RBD proteins for ELISA and/or flow cytometry has been previously described in detail (Juno et al., 2020b). A construct for expressing SARS-CoV-2 with six proline stabilisation mutations (Hexapro) was synthesised and expressed as previously described (Hsieh et al., 2020). A construct for expressing NTD (residues 1-290) was synthesised with a C-terminal Avitag and polyhistidine tag. A construct for expressing SARS-CoV spike protein was synthesised containing 2P mutations and a C-terminal trimerization foldon, Avitag and polyhistidine tag. All coronavirus proteins were expressed using transient transfection of Expi293 or ExpiCHO cells and purified by Ni-NTA affinity and size-exclusion chromatography using a Superdex S75 increase 10/300 column or a Superose 6 16/70 column (Cytiva).

### Limiting Dilution Microneutralisation Assay

SARS-CoV-2 isolate CoV/Australia/VIC01/2020 was passaged in Vero cells and stored at −80 C. Plasma was heat-inactivated at 56° C. for 30 min. Plasma was serially diluted 1:20 to 1:10240 before addition of 100 TCID50 of SARS-CoV-2 in MEM/0.5% BSA and incubation at room temperature for 1 hour. Residual virus infectivity in the plasma/virus mixtures was assessed in quadruplicate wells of Vero cells incubated in serum-free media containing 1 μg/ml TPCK trypsin at 37° C./5% CO2; viral cytopathic effect was read on day 5 and neutralising antibody titre calculated using the Reed/Muench method as described (Subbarao et al., 2004).

Recovery of Human Monoclonal Antibodies from SARS-CoV-2-Specific B Cells

Fluorescent B cell probes for identification of SARS-CoV-2 S-specific B cells within cryopreserved human PBMC were generated as described (Juno et al., 2020a). Cells were stained with Aqua viability dye (ThermoFisher) before incubation with B cell probes (Hexapro spike or RBD) and monoclonal antibodies for surface staining CD19-ECD (J3-119) (Beckman Coulter), CD20 Alexa700 (2H7), IgM-BUV395 (G20-127), CD21-BUV737 (B-ly4), IgD-Cy7PE (IA6-2), IgG-BV786 (G18-145) (BD), CD14-BV510 (M5E2), CD3-BV510 (OKT3), CD8a-BV510 (RPA-T8), CD16-BV510 (3G8), CD10-BV510 (H110a) (Biolegend). Single antigen-specific class-switched B cells (S or RBD+, CD19+IgD−IgG+) were sorted using a BD Aria II into 96-well plates, subject to cDNA generation and multiplex PCR and Sanger sequencing, as previously described (Juno et al., 2020a; Tiller et al., 2008). Productive, recombined heavy (V-D-J) and light (V-J) chain immunoglobulin sequences were synthesised (Geneart) and cloned into human IgG1 expression vectors for recombinant production in Expi293 mammalian cell culture using transient transfection. After 4-5 days, IgG1 was purified from culture supernatants using Protein-A affinity chromatography.

### Phage Library Isolation of SARS-CoV-2 Spike and RBD Antibodies

Biopanning for SARS-CoV-2 spike and RBD human antibodies displayed in a Human antibody phage library was performed as previously described (Panousis et al., 2016). Phages displaying SARS-CoV-2 spike and RBD specific Fabs were enriched after three rounds of biopanning on biotinylated SARS-CoV-2 spike or RBD protein immobilised to streptavidin Dynabeads (Dynal M-280, Invitrogen, cat #112.06D). After the third round of panning, individual clones were selected for further analyses by ELISA for the presence of SARS-CoV-2 spike and RBD binding phage respectively. Positive clones were sequenced and annotated using the International ImMunoGeneTics database (IMGT) and aligned in Geneious Prime. Fabs from positive phage were reformatted into IgG1 expression plasmids and used to transiently transfect Expi293 cells. Human IgG1 antibodies were purified using Protein-A affinity chromatography.

Screening ELISA for Phage Library mAbs

96-well flat-bottomed MaxiSorp plates were coated with 50 μl of 125 nM recombinant protein in PBS at room temperature for one hour. All washes were done three times using PBS and 0.1% Tween (DPBS-T) and all incubations were performed for one hour at room temperature. Coated plates were washed and blocked by incubation with 4% skim milk solution. Plates were washed and then incubated with 50 μl of 125 nM IgG1. The plates were washed and incubated with horseradish peroxidase (HRP)-conjugated Goat anti-Human IgG secondary antibody (1:5000). After a final wash, 50 μL of azino-bis-3-ethylbenthiazoline-6-sulfonic acid (ABTS liquid substrate; Sigma) was added and incubated in the dark at room temperature for 20 minutes and 50 μL of 1% SDS was used to stop the reaction. Absorbance was read at 405 nm and all samples were done in duplicate.

### Assessment of mAb Binding Specificity by ELISA

96-well Maxisorp plates (Thermo Fisher) were coated overnight at 4° C. with 2 μg/mL recombinant SARS-CoV-2 S, SARS-CoV-2 RBD, SARS-CoV-2 NTD, SARS-CoV S, HKU-1 S or OC43 S proteins. After blocking with 1% FCS in PBS, antibodies diluted in PBS incubated for two hours and then washed prior to incubation with 1:20000 dilution of HRP-conjugated anti-human IgG (Sigma) for 1 hour. Plates were washed and developed using TMB substrate (Sigma), stopped using 0.16 M sulphuric acid and read at 450 nm. Effective concentration midpoints (EC50) concentrations were calculated using a fitted curve (4 parameter log regression) and Prism 9.0 software (Graphpad).

### ACE2-RBD Inhibition ELISA

An ELISA was performed to measure the ability of antibodies to block interaction between recombinant human ACE2 and RBD proteins. 96-well Maxisorp plates (Thermo Fisher) were coated overnight at 4° C. with 2.5 pg/ml of recombinant RBD protein in carbonate-bicarbonate coating buffer (Sigma). After blocking with PBS containing 1% BSA, duplicate wells of 2.5-fold serially diluted mAbs (from 5 μg/ml) were added and incubated for 1 hour at room temperature. Plates were then incubated with 1 μg/ml of biotinylated recombinant ACE2 protein for 1 hour at room temperature followed by incubation with HRP-conjugated streptavidin (ThermoFisher Scientific) for hour at room temperature. Plates were developed with TMB substrate (Sigma), stopped with 0.15 M sulphuric acid and read at 450 nm.

Microneutralisation Assay with ELISA-Based Read Out

Wildtype SARS-CoV-2 (CoV/Australia/VIC/01/2020) and B.1.351 (CoV/Australia/QLD/1520/2020) isolates were passaged in Vero cells and stored at −80° C. 96-well flat bottom plates were seeded with Vero cells (20,000 cells per well in 100 μl). The next day, Vero cells were washed once with 200 μl serum-free DMEM and added with 150 μl of infection media (serum-free DMEM with 1.33 pg/ml TPCK trypsin). 2-fold serial dilutions of mAbs (from 5 μg/ml) were incubated with WT and B.1.351 SARS-CoV-2 isolates at 2000 TCID₅₀/ml at 37° C. for 1 hour. Next, mAb-virus mixtures (50 μl) were added to Vero cells in duplicate and incubated at 37° C. for 48 hours. ‘Cells only’ and ‘virus+cells’ controls were included to represent 0% and 100% infectivity respectively. After 48 hours, all cell culture media were carefully removed from wells and 200 μl of 4% formaldehyde was added to fix the cells for 30 mins at room temperature. The plates were then dunked in a 1% formaldehyde bath for 30 minutes to inactivate any residual virus prior to removal from the BSL3 facility. Cells were washed once in PBS and then permeabilised with 150 μl of 0.1% Triton-X for 15 minutes. Following one wash in PBS, wells were blocked with 200 μl of blocking solution (4% BSA with 0.1% Tween-20) for 1 hour. After three washes in PBST (PBS with 0.05% Tween-20), wells were added with 100 μl of rabbit polyclonal anti-SARS-CoV N antibody (Rockland, #200-401-A50) at a 1:8000 dilution in dilution buffer (PBS with 0.2% Tween-20, 0.1% BSA and 0.5% NP-40) for 1 hour. Plates were then washed six times in PBST and added with 100 μl of goat anti-rabbit IgG (Abcam, #ab6721) at a 1:8000 dilution for hour. After six washes in PBST, plates were developed with TMB and stopped with 0.15M H₂SO₄. OD values read at 450 nm were then used to calculate % neutralisation with the following formula: (‘Virus+cells’−‘sample’)÷(‘Virus+cells’−‘Cells only’)×100. IC50 values were determined using four-parameter nonlinear regression in GraphPad Prism with curve fits constrained to have a minimum of 0% and maximum of 100% neutralisation.

### Affinity Measurements Using Bio-Layer Interferometry

Affinity determination measurements were performed on the Octet RED96e (FortéBio). Assays were performed at 25° C. in solid black 96-well plates agitated at rpm. Kinetic buffer was composed of PBS pH 7.4 supplemented with 0.1% (w/v) BSA and 0.05% (v/v) TWEEN-20. All assays were performed using anti-human IgG Fc capture sensor tips (AHC) sensors (FortéBio). A 60 s biosensor baseline step was applied before human antibodies (5 pg/mL) were loaded onto AHC sensors. For affinity measurements against SARS-CoV-2 RBD, antibodies were loaded by submerging sensor tips for 200 s and then washed in kinetics buffer for 60 s. Association measurements were performed by dipping into a two-fold dilution series of SARS-CoV-RBD from 6-200 nM for 180 s and dissociation was measured in kinetics buffer for 180 s. For affinity measurements against SARS-CoV-2 spike, antibodies were loaded by submerging sensor tips until a response of 0.5 nm then washed in kinetics buffer for 60 s. Association measurements were performed by dipping into a two-fold dilution series of SARS-CoV-2 spike from 3-100 nM for 180 s and dissociation was measured in kinetics buffer for 180 s. Sensor tips were regenerated using a cycle of 5 s in 10 mM glycine pH 1.5 and 5 s in kinetic buffer repeated five times. Baseline drift was corrected by subtracting the average shift of an antibody loaded sensor not incubated with protein and an unloaded sensor incubated with protein. Curve fitting analysis was performed with Octet Data Analysis 10.0 software using a global fit 1:1 model to determine K_{D} values and kinetic parameters. Curves that could not be fitted were excluded from the analyses. Mean kinetic constants and standard error of the mean reported are the result of three independent experiments.

### Two-Way Competition Bio-Layer Interferometry for Epitope Assignment

In antibody competition experiments using bio-layer interferometry, NTA sensors were loaded with 3 μg/mL of SARS-CoV-2 RBD for 5 min. After loading, sensor tips were first submerged into wells containing 200 nM of the first antibody for 10 min then subsequently dipped into wells containing 200 nM of a second antibody for 5 min. One SARS-CoV-2 RBD loaded sensor was dipped only into the second antibody to determine the maximum response in the absence of the first antibody binding. Sensor tips were regenerated using a cycle of 5 s in 300 mM imidazole pH 7.5 and 5 s in kinetic buffer repeated five times. Competition was calculated by dividing the maximum response of the second antibody binding to immobilized SARS-CoV-2 RBD in the presence of the first antibody by the maximum response of the second antibody binding in the absence of the first antibody.

### Plaque Reduction Neutralization Assay

Plaque reduction neutralisation titres (PRNT) were calculated using clinical isolates of SARS-CoV-2 hCoV-19/Australia/VIC01/2020 and hCoV-19/Australia/VIC2089/2020 (D614G/N501Y). Duplicate two-fold serial dilutions of antibody were prepared in DME media (ThermoFisher) and combined with an equal volume of DME media+2 μg/ml trypsin TPCK (ThermoFisher) containing 125 TCID50 SARS-CoV-2. The antibody/virus mixture was incubated at room temperature for one hour before plating onto confluent monolayers of Vero cells (clone CCL81) in 24-well plates and a further incubation at 37° C. supplied with 5% CO₂ for 1 hour. 0.6 mL of DME media containing 4% FBS+1.5% (w/v) methylcellulose (Sigma) was added to each well and plates were incubated for five days at 37° C. supplied with 5% CO₂. Plaques were visualised and counted by staining with 0.2% (w/v) crystal violet after fixation in 4% formaldehyde. IC50 values were calculated using four-parameter logistic regression using GraphPad Prism 8.0 (GraphPad Software Inc).

### Variant RBD Multiplex Array

A multiplex array consisting of reported RBD variants (n=24) was used to examine the epitope resilience of human mAbs as previously described (Pymm et al., 2021). Briefly RBD variant multiplex bead cocktails were generated. Bead cocktails (1000 beads of each region per well) and mAbs were added at 8-fold 1:4 titrations, from a starting concentration of 80 nM per well into 384 well plates. Plates were incubated for 2 h with shaking, washed twice with 0.05% PBS Tween and relative mAb binding was detected using anti-human IgG-PE (#9040-09, Southern Biotech) at 1.3ug/ml for 2 hours with shaking.

Relative ACE2 inhibition was conducted using the same RBD variant multiplex bead cocktail and mAbs titrated, as described above, with the addition of 25ug/ml biotinylated ACE2 per well. Plates were incubated for 2 h with shaking, washed twice with 0.05% PBS Tween followed by the addition of Streptavidin-PE (#S866, Thermo Fisher) and PE Biotin-XX conjugate (#P811, Thermo Fisher) for another 1 h with shaking. Plates were acquired on a Flexmap3D™ (Luminex Corporation). The relative binding of mAb or ACE2, was detected as phycoerythrin-labelled reporter and is measured as MFI (Median Fluorescence Intensity)

### Prophylaxis Studies in Mice Using SARS-CoV-2 D614G N501Y Virus

C57BL/6J mice were bred and housed at the Walter and Eliza Hall Institute of Medical Research. All procedures involving animals and live SARS-CoV-2 were conducted in an OGTR-approved Physical Containment Level 3 (PC-3) facility at the Walter and Eliza Hall Institute of Medical Research (Cert-3621; IA88_20). All animal procedures were approved by The Walter and Eliza Hall Institute of Medical Research Animal Ethics Committee (2020.016). Human antibodies were administered to mice in pL phosphate buffered saline by intraperitoneal injection 24 hours prior to infection. SARS-CoV-2 infection (clinical isolate hCoV-19/Australia/VIC2089/2020) of C57BL/6J mice was performed using an inhalation exposure system (Glas-Col, LLC) for 45 minutes loaded with 1.5×10⁷ SARS-CoV-2 TCID50. Mice used for experimentation were 7-10 weeks of age.

### Measurement of Viral Burden in SARS-CoV-2 Infected Mice

Three days post-infection, mice were humanely killed and lungs removed and homogenised in a Bullet Blender (Next Advance Inc) in 1 mL DME media (ThermoFisher) containing steel homogenisation beads (Next Advance Inc). Samples were clarified by centrifugation at 10,000×g for 5 minutes before virus quantification by TCID50 assays. SARS-CoV-2 live virus quantification by TCID50 assay: SARS-CoV-2 lung TCID50 was determined by plating 1:7 serially-diluted lung tissue homogenate onto confluent layers of Vero cells (clone CCL81) in DME media (ThermoFisher) containing 0.5 pg/ml trypsin-TPCK (ThermoFisher) in replicates of six on 96-well plates. Plates were incubated at 37° C. supplied with 5% CO₂ for four days before measuring cytopathic effect under light microscope. The TCID50 calculation was performed using the Spearman and Kärber method.

### Prophylaxis Studies in Hamsters

Groups 8-wk-old Syrian hamsters were obtained from Envigo and housed under ABSL3 containment. Animals were administered 5 mg/kg or 0.25 mg/kg human mAbs as single agents or cocktails via intraperitoneal injection. Animals were subsequently challenged 24 hours later under ketamine-xylazine anaesthesia by intranasal instillation of 100 μl of SARS-CoV-2 (strain SARS-CoV-2/human/USA/WA-CDC-WA1/2020). From 2 days prior to challenge until euthanasia, hamsters were evaluated clinically and weighed. An equal number of hamsters from each treatment group was necropsied 3 d after challenge, ˜100 mg of right cranial and right caudal lung lobes and nasal tissues was excised, immersed in 0.9 ml of BA1/FBS, and homogenized. Tissue homogenates were frozen at −80° C. Virus titrations were performed using a double-overlay plaque assay on Vero E6 cells in 6-well plates. Briefly, serial 10-fold dilutions of tissue homogenates were inoculated onto cells and incubated 45 min, and 2 ml of a first overlay (0.5% agarose in MEM) without neutral red was added to each well. A second 2-ml overlay containing 0.06 mg/ml neutral red was added after 1 day and plaques were counted after an additional 1 and 2 d.

### Crystallociraphy

Fab fragments were generated from full length IgG1 antibodies through cleavage with IgG degrading enzyme E (IGDE) for 16-24 hours at 37° C. Fab fragments were purified using Lambda and Kappa Select columns (Cytiva) per the manufacturer's protocol. Complexes of SARS-CoV-2 RBD bound to Fab fragments were purified via SEC in 20 mM HEPES pH 7.5, 150 mM NaCl. Crystallization trials were set up with protein concentrations of 8 and 4 mg/ml at the Collaborative Crystallization Centre at Commonwealth Scientific and Industrial Research Organisation (CSIROC3, Parkville) at 20° C. Crystals of SARS-CoV-2 RBD-PDI 37 appeared in 0.2 M ammonium sulphate, 20% PEG3350, 0.1 M Tris-chloride pH 8.5 and were harvested with mother liquor containing 25% glycerol. SARS-CoV-2 RBD-PDI 42 crystals were obtained in 10% PEG8000, 0.2 M NaCl, 0.1 M sodium dihydrogen-dipotassium hydrogen phosphate pH 6.2 and were flash frozen in mother liquor containing 30% glycerol. Crystals of SARS-CoV-2 RBD-PDI 210 appeared in 15% PEG6000, 0.1% (w/v) n-Octyl-b-D-glucoside and crystals of SARS-CoV-2 RBD-WCSL 129- PDI 96 grew in 0.1 M Tri-sodium citrate pH 5.5, 10% PEG8000. These crystals were flash frozen in mother liquor containing 20% glycerol or 20% 2-Methyl-2,4-pentanediol, respectively.

Hanging drop vapour diffusion crystallization trials were performed in-house for crystal optimization of SARS-CoV-2 RBD bound to WCSL 119, WCSL 129, WCSL 129- PDI 93, PDI 215, and PDI 231. Diffraction quality crystals of SARS-CoV-2 RBD-WCSL 119 crystals grew in 14% PEG3350, 0.2 M potassium thiocyanate seeded from initial screens and were stepwise transferred into cryo-protectant containing 30% ethylene glycol. SARS-CoV-2 RBD-WCSL 129 were obtained in 18% PEG3350, 0.1 M tri sodium citrate pH 5.5 seeded from initial screens and were harvested with 30% glycerol in mother liquor. A crystallization plate of SARS-CoV-2 RBD-PDI 215 was incubated at 4° C. for 48 hours and subsequently transferred to 20° C. Crystals appeared after 2 days in 12% isopropanol, 12% PEG4000, 0.1 M tri sodium citrate pH 5.6 seeded from initial screens and were flash frozen in mother liquor containing 30% glycerol. SARS-CoV-2 RBD-WCSL 129- PDI 93 crystals grew in 23% PEG3350, 0.1 M sodium acetate pH 4.5 seeded from initial screens and SARS-CoV-2 RBD-PDI 231 crystals in 18% PEG3350, 10% N,N-Dimethyldodecylamine N-oxide (LDAO), 0.2 M sodium sulphate seeded from initial screens. Mother liquor containing 6% glycerol was used as cryo-protectant for SARS-CoV-2 RBD-WCSL 129-PDI 93 and mother liquor containing 20% butanediol was used for SARS-CoV-2 RBD-PDI 231 crystals.

X-ray diffraction data was collected at the MX2 beamline at the Australian Synchrotron, recorded with an Eiger 16M detector (Dectris) and processed using the XDS package. Molecular replacement using Phaser was performed to solve the phase problem. SARS-CoV-2 RBD (PDB ID 6W41) and Fab structures of high sequence similarity were use as search models (PDB ID 6XC4 (heavy chain, HC) and 6UTA (light chain, LC) for PDI 37, for PDI 42, 7JXC for PDI 93, 3N9G for PDI 96, 51ES (heavy chain, HC) and 5WL2 (light chain, LC) for WCSL 119, 5HHV (HC) and 6A67 (LC) for WCSL 129, 6MHR for PDI 210, 6PE7 for PDI 215, 7CHB (HC) and 6PHB (LC) for PDI 231). For complexes of SARS-2 RBD bound by two Fabs simultaneously (WCSL 129-PDI 93, or WCSL 129-PDI 96) coordinates of the previously solved SARS-CoV-2 RBD-WCSL 129 structure was used as a search model instead of PDB ID 6W41. Iterative rounds of model building and refinement were undertaken using COOT (Emsley et al., 2010) and Phenix (Adams et al., 2010). Figures of the complexes were prepared using the PyMOL Molecular Graphics System, Version 2.3.0 (Schrôdinger, LLC). Interactions, interfaces and buried areas from solvent were analysed using PDBePISA v1.52 (Krissinel and Henrick, 2007). The atomic coordinates and structure factor files have been deposited in the Protein Data Bank. Accession numbers are listed in the crystallographic Table 1.

TABLE 1
Data collection and refinement statistics for SARS CoV-2 RBD FAb complexes

| | | | | | | | | | SARS-CoV-2 | |
|---|---|---|---|---|---|---|---|---|---|---|
| | SARS-CoV-2 | | SARS-CoV-2 | | SARS-CoV-2 | | SARS-CoV-2 | | RBD-WCSL 129- | |
| | RBD-PDI 37 | | RBD-PDI 42 | | RBD-WCSL 119 | | RBD-WCSL 129 | | PDI 93 | |
| PDB ID | 7MZF | | 7MZG | | 7MZH | | 7MZI | | 7MZJ | |
| Data collection | | | | | | | | | | |
| Space group | C 2 2 2₁ | | C 2 2 2₁ | | P 1 2₁ 1 | | I 1 2 1 | | P 1 | |
| Cell dimensions | | | | | | | | | | |
| a, b, c (Å) | 83.92, 147.66, | | 82.27, 150.77, | | 93.70, 72.83, | | 106.57, 71.89, | | 39.04, 104.72, | |
| | 146.10 | | 145.90 | | 103.99 | | 113.69 | | 127.27 | |
| α, β, γ (°) | 90.00, 90.00, | | 90.00, 90.00, | | 90.00, 90.71, | | 90.00, 108.32, | | 88.72, 83.68, | |
| | 90.00 | | 90.00 | | 90.00 | | 90.00 | | 82.80 | |
| Resolution (Å) | 48.70-2.49 | | 48.63-2.00 | | 46.85-2.10 | | 44.55-1.85 | | 48.20-2.40 | |
| | (2.64-2.49) | | (2.12-2.00) | | (2.14-2.10) | | (1.89-1.85) | | (2.54-2.40) | |
| Total no. of | 325436 | (50938) | 426407 | (68231) | 416245 | (22048) | 243523 | (15191) | 278333 | (43673) |
| reflections | | | | | | | | | | |
| No. of unique | 31884 | (4951) | 61570 | (9662) | 81929 | (4526) | 69190 | (4230) | 76559 | (12144) |
| reflections | | | | | | | | | | |
| Rₘₑₐₛ (%) | 18.0 | (166.8) | 10.9 | (103.2) | 8.1 | (92.0) | 9.3 | (96.6) | 11.3 | (93.0) |
| Rₚᵢₘ (%) | 5.6 | (51.3) | 4.1 | (38.1) | 3.5 | (41.1) | 4.9 | (50.4) | 5.9 | (49.1) |
| I/σ(I) | 11.3 | (1.4) | 11.8 | (1.5) | 11.8 | (1.8) | 9.6 | (1.6) | 9.3 | (1.5) |
| CC_{1/2} | 99.7 | (64.1) | 99.8 | (76.6) | 99.9 | (80.5) | 99.8 | (68.0) | 99.7 | (64.9) |
| Completeness (%) | 99.5 | (96.9) | 99.7 | (98.1) | 99.9 | (99.9) | 99.3 | (98.6) | 98.4 | (97.0) |
| Redundancy | 10.2 | (10.3) | 6.9 | (7.1) | 5.1 | (4.9) | 3.5 | (3.6) | 3.6 | (3.6) |
| Wilson B (Å²) | 55.4 | | 40.4 | | 40.4 | | 22.6 | | 52.7 | |
| Refinement | | | | | | | | | | |
| Resolution (Å) | 42.46-2.49 | | 40.87-2.00 | | 45.70-2.10 | | 44.55-1.85 | | 48.20-2.40 | |
| No. reflections | 31873 | (2997) | 61441 | (6050) | 81788 | (8117) | 69174 | (6799) | 76530 | (7578) |

| | | | | | |
|---|---|---|---|---|---|
| R_{work}/R_{free} (%) | 18.51/22.72 | 20.03/23.64 | 21.99/26.53 | 17.40/20.75 | 22.21/26.82 |
| No. atoms | | | | | |
| Protein | 4761 | 4697 | 9478 | 4739 | 15619 |
| Ligand/ion | 50 | 74 | 99 | 136 | 84 |
| Water | 162 | 456 | 226 | 426 | 258 |
| B factors | | | | | |
| Protein | 54 | 43 | 49 | 34 | 55 |
| Ligand/ion | 94 | 74 | 82 | 65 | 69 |
| Water | 53 | 47 | 46 | 39 | 46 |
| R.m.s. deviations | | | | | |
| Bond lengths (Å) | 0.004 | 0.003 | 0.009 | 0.012 | 0.003 |
| Bond angles (°) | 0.693 | 0.646 | 1.081 | 1.120 | 0.504 |
| Validation | | | | | |
| MolProbity score | 1.35 | 1.21 | 1.42 | 1.27 | 1.56 |
| Clashscore | 2.70 | 2.59 | 3.75 | 3.06 | 4.71 |
| Poor rotamers (%) | 0.00 | 0.20 | 0.00 | 0.57 | 0.00 |
| Ramachandran | | | | | |
| plot | | | | | |
| Favored (%) | 95.78 | 97.08 | 96.22 | 96.95 | 95.35 |
| Allowed (%) | 4.22 | 2.92 | 3.78 | 3.05 | 4.65 |
| Disallowed (%) | 0.00 | 0.00 | 0.00 | 0.00 | 0.00 |

| | SARS-CoV-2 | | | | | | | |
|---|---|---|---|---|---|---|---|---|
| | RBD-WCSL | | SARS-CoV-2 | | SARS-CoV-2 | | SARS-CoV-2 | |
| | 129-PDI 96 | | RBD-PDI 210 | | RBD-PDI 215 | | RBD-PDI 231 | |
| PDB ID | 7MZK | | 7MZL | | 7MZM | | 7MZN | |
| Data collection | | | | | | | | |
| Space group | P 1 | | P 4 2₁ 2 | | C 2 2 2₁ | | P 2₁ 2₁ 2₁ | |
| Cell dimensions | | | | | | | | |
| a, b, c (Å) | 71.90, 103.56, | | 177.56, 177.56, | | 95.80, 120.33, | | 55.48, 103.71 | |
| | 106.29, | | 68.57 | | 128.36 | | 163.20 | |
| α, β, γ (°) | 103.52, 101.82, | | 90.00, 90.00, | | 90.00, 90.00, | | 90.00, 90.00, | |
| | 98.20 | | 90.00 | | 90.00 | | 90.00 | |
| Resolution (Å) | 49.33-2.25 | | 46.3-3.70 | | 48.75-2.30 | | 48.92-3.10 | |
| | (2.38-2.25) | | (4.05-3.70) | | (2.44-2.30) | | (3.31-3.10) | |
| Total no. of | 488458 | (78995) | 157692 | (37093) | 452899 | (72272) | 117965 | (20669) |
| reflections | | | | | | | | |
| No. of unique | 133496 | (21202) | 12240 | (2874) | 33371 | (5266) | 17774 | (3143) |
| reflections | | | | | | | | |
| Rₘₑₐₛ (%) | 12.6 | (85.8) | 35.8 | (136.2) | 17.1 | (168.6) | 30.3 | (134.7) |
| Rₚᵢₘ (%) | 6.5 | (44.1) | 9.9 | (37.8) | 4.6 | (44.6) | 11.6 | (52.1) |
| I/σ(I) | 8.1 | (1.6) | 7.5 | (2.3) | 11.6 | (2.0) | 6.5 | (1.5) |
| CC_{1/2} | 99.5 | (67.0) | 99.5 | (76.4) | 99.7 | (66.0) | 98.9 | (74.3) |
| Completeness (%) | 98.2 | (96.8) | 99.9 | (100.0) | 99.8 | (98.8) | 99.9 | (99.9) |
| Redundancy | 3.7 | (3.7) | 12.9 | (12.9) | 13.6 | (13.7) | 6.6 | (6.6) |
| Wilson B (Å²) | 45.0 | | 71.2 | | 52.7 | | 63.9 | |
| Refinement | | | | | | | | |
| Resolution (Å) | 49.33-2.25 | | 46.30-3.70 | | 47.90-2.30 | | 48.92-3.10 | |
| No. reflections | 133432 | (13187) | 12207 | (1199) | 33293 | (3292) | 17687 | (1690) |

| | | | | |
|---|---|---|---|---|
| R_{work}/R_{free} (%) | 19.72/23.51 | 22.15/25.62 | 21.60/25.73 | 22.33/25.85 |
| No. atoms | | | | |
| Protein | 15891 | 4543 | 4841 | 4799 |
| Ligand/ion | 115 | 38 | 32 | 43 |
| Water | 797 | n/a | 107 | n/a |
| B factors | | | | |
| Protein | 51 | 102 | 53 | 64 |
| Ligand/ion | 79 | 162 | 97 | 141 |
| Water | 45 | n/a | 47 | n/a |
| R.m.s. deviations | | | | |
| Bond lengths (Å) | 0.002 | 0.001 | 0.004 | 0.002 |
| Bond angles (°) | 0.522 | 0.447 | 0.75 | 0.483 |
| Validation | | | | |
| MolProbity score | 1.42 | 1.52 | 1.34 | 1.47 |
| Clashscore | 4.74 | 3.68 | 2.66 | 3.61 |
| Poor rotamers | 0.12 | 0.22 | 0.96 | 0.00 |
| (%) | | | | |
| Ramachandran | | | | |
| plot | | | | |
| Favored (%) | 96.92 | 94.78 | 95.71 | 95.40 |
| Allowed (%) | 3.08 | 5.22 | 4.29 | 4.60 |
| Disallowed (%) | 0.00 | 0.00 | 0.00 | 0.00 |

X-ray diffraction data were collected on single crystals.
*Values in parentheses are for highest-resolution shell.

Cryo-EM sample preparation and data acquisition

The pre-fusion stabilised spike trimer (Hexapro) was purified over SEC and concentrated to 2.5 mg/ml. Fab fragments of human IgG1 antibodies PDI 93, PDI 96, PDI 210, PDI 215, PDI 222, WCSL 119 and WCSL 129 were purified over SEC and concentrated to 6 mg/ml. Prior to grid preparation spike trimer was incubated with a Fab fragment at a 4:1 molar ratio for 30 minutes at room temperature. The grids (Quantifoil Cu R1.2/1.3) were glow discharged in air at 10 mA for 90 s using Pelco EasyGlow. The 3 μl samples were applied to the grids at 4° C. and 100% humidity and plunge frozen in liquid ethane using Vitrobot Mark IV (Thermo Fisher Scientific, USA). Data was collected on Titan Krios (Thermo Fisher Scientific, USA) 300 kV electron microscope using K2 detector (Gatan, USA). The data were collected during three sessions.

In the first session datasets for antibody-Spike complexes for candidates PDI 96, PDI 210, WCSL 129, PDI 93 and PDI 222 were collected with 1049, 1008, 1003, 1063 and 723 movies respectively, each movie containing 50 frames at a pixel size of 1.8 Å and total exposure of 50.0 e/Å². In the second session datasets for antibody-Spike complexes for candidates PDI 215 and WCSL 119 were collected with and 1866 movies respectively, each movie containing 40 frames at a pixel size of 1.8 Å and total exposure of 52.2 e/Å². The acquisition for these two sessions was performed at 81 K indicated magnification using the energy filter slit width of 15 eV. In the third session a higher resolution dataset was collected for the PDI 222 antibody-Spike complex. This dataset was collected with 1900 movies, each movie containing frames at a pixel size of 1.06 Å and total exposure of 50.0 e/Å². The acquisition was performed at 130K indicated magnification using the energy filter slit width of 15 eV. All data were collected using FEI EPU software using a 9-hole beam-image shift acquisition scheme with one exposure in each hole.

### Cryo-EM Data Processing

### Processing of Medium-Resolution Datasets for PDI 96, PDI 210, WCSL 129, PDI 93 and PDI 222

All datasets were processed independently. Data collected in the first two sessions at moderate resolution (1.8 Å pixel size) were subjected to the correction of beam-induced motion using MotionCor2 (Zheng et al., 2017), followed by CTF estimation using Gctf (Zhang, 2016). Micrographs with CTF fit resolution below 6 Å were selected for further examination. Template-based particle picking was performed using Gautomatch (http://www.mrc-Imb.cam.ac.uk/kzhang/) using projections from EMDB 23566 low-pass filtered to 20 Å. Particle coordinates were imported into Relion 3.1 (Zivanov et al., 2018). Particle extraction using 2× binned particles (112 pixel box size) yielded 892,966 (PDI 96), 939,575 (PDI 210), 959,431 (WCSL 129), 994,647 (PDI 93), 709,466 (PDI 222), 1,925,957 (WCSL 119) and 2,089,347 (PDI 215) particles.

All further processing for PDI 96, PDI 210, WCSL 129, PDI 93 and PDI 222 was performed in Relion 3.1. First, particles underwent 2D classification, good classes were selected and proceeded to 3D classification. Particles from the best 3D class or classes were reextracted without rescaling (224 pixel box size) and subjected to 3D refinement, resulting in datasets containing 214,450 (PDI 96), 381,942 (PDI 210), 238,812 (WCSL 129), 236,164 (PDI 93) and 176,819 (PDI 222) particles. Following post-processing, the final datasets gave maps in C1 symmetry between 4.07 Å and 4.43 Å (Table 2) based on the gold standard Fourier shell correlation cut-off of 0.143. Local resolution was determined using the internal local resolution procedure in Relion, using half-reconstructions as input maps.

TABLE 2
Summary of interactions between SARS-
CoV-2 RBD and human antibodies.
SARS CoV-2 RBD and PDI 37 interactions

| | | | | |
|---|---|---|---|---|
| SARS CoV- | | PDI 37 | | Distance |
| 2 RBD | Group | V_{H} | Group | (Å) |

Hydrogen bonds

| | | | | |
|---|---|---|---|---|
| Thr 415 | OG1 | Tyr 58 | OH | 2.7 |
| Lys 417 | N | Tyr 52 | OH | 3.8 |
| Lys 417 | NZ | Asp 98 | OD1 | 3.4 |
| Asp 420 | OD2 | Thr 56 | OG1 | 2.6 |
| Tyr 421 | OH | Ser 53 | N | 3.5 |
| Tyr 421 | OH | Ser 53 | OG | 3.4 |
| Tyr 421 | OH | Gly 54 | N | 2.9 |
| Tyr 453 | OH | Asp 98 | OD2 | 2.7 |
| Leu 455 | O | Tyr 33 | OH | 2.6 |
| Arg 457 | O | Ser 53 | OG | 2.6 |
| Lys 458 | O | Ser 53 | OG | 3.5 |
| Tyr 473 | OH | Ser 53 | OG | 3.8 |
| Tyr 473 | OH | Arg 31 | O | 2.6 |
| Gln 474 | O | Arg 31 | NE | 2.9 |
| Ala 475 | O | Asn 32 | ND2 | 2.9 |
| Ala 475 | O | Ile 28 | N | 3.3 |
| Ser 477 | N | Glu 26 | O | 2.8 |
| Ser 477 | OG | Glu 26 | O | 3.4 |
| Thr 478 | N | Glu 26 | OE1 | 3.7 |
| Thr 478 | OG1 | Glu 26 | OE1 | 3.7 |
| Asn 487 | OD1 | Arg 94 | NH1 | 3.0 |
| Tyr 489 | OH | Arg 94 | NH2 | 2.8 |

Salt bridges

| | | | | |
|---|---|---|---|---|
| Lys 417 | NZ | Asp 98 | OD1 | 3.4 |

Other SARS CoV-2 interfacing residues (PDI 37 V_{H})

| | | | | |
|---|---|---|---|---|
| Phe 27 | Arg 96 | Gly 97 | Asp 101 | Tyr 102 |

Other PDI 37 V_{H} interfacing residues (SARS CoV-2)

| | | | | |
|---|---|---|---|---|
| Gly 416 | Phe 456 | Asn 460 | Gly 476 | Phe 486 |

SARS CoV-2 RBD and PDI 37 interactions

| | | | | |
|---|---|---|---|---|
| SARS CoV- | | PDI 37 | | Distance |
| 2 RBD | Group | V_{L} | Group | (Å) |

Hydrogen bonds

| | | | | |
|---|---|---|---|---|
| Arg 403 | NH1 | Asn 92 | O | 2.9 |
| Arg 403 | NH1 | Asn 92 | OD1 | 3.5 |
| Lys 417 | NZ | Asn 92 | OD1 | 3.5 |
| Tyr 453 | OH | Asn 92 | ND2 | 3.4 |
| Gln 498 | OE1 | Ser 30 | OG | 3.8 |
| Gln 498 | OE1 | Ser 31 | OG | 3.6 |
| Thr 500 | OG1 | Ser 28 | OG | 3.2 |
| Thr 500 | OG1 | Glu 68 | OE1 | 3.0 |
| Asn 501 | N | Glu 68 | OE2 | 3.0 |

Other SARS CoV-2 interfacing residues (PDI 37 V_{L})

| | | | | |
|---|---|---|---|---|
| Ile 2 | Ile 29 | Trp 32 | Ser 93 | Tyr 94 |

Other PDI 37 V_{H} interfacing residues (SARS CoV-2)

| | | | |
|---|---|---|---|
| Arg 408 | Tyr 495 | Gly 502 | Tyr 505 |

SARS CoV-2 RBD and PDI 42 interactions

| | | | | |
|---|---|---|---|---|
| SARS CoV- | | PDI 37 | | Distance |
| 2 RBD | Group | V_{H} | Group | (Å) |

Hydrogen bonds

| | | | | |
|---|---|---|---|---|
| Thr 415 | OG1 | Tyr 58 | OH | 2.54 |
| Asp 420 | OD2 | Ser 56 | OG | 2.71 |
| Tyr 421 | OH | Gly 54 | N | 2.84 |
| Leu 455 | O | Tyr 33 | OH | 2.70 |
| Tyr 473 | OH | Ser 31 | O | 2.67 |
| Ala 475 | O | Thr 28 | N | 3.11 |
| Ala 475 | O | Asn 32 | ND2 | 3.00 |
| Glu 484 | OE2 | Tyr 102 | OH | 2.80 |
| Asn 487 | ND2 | Gly 26 | O | 2.85 |
| Asn 487 | OD1 | Arg 97 | NH1 | 2.91 |
| Asn 487 | OD1 | Arg 97 | NH2 | 2.71 |
| Phe 490 | N | Tyr 102 | OH | 3.67 |
| Gln 493 | NE2 | Tyr 102 | OH | 3.47 |

Other SARS CoV-2 interfacing residues (PDI 42 V_{H})

| | | | | |
|---|---|---|---|---|
| Val 2 | Gly 26 | Leu 27 | Thr 28 | Ser 31 |
| Asn 32 | Tyr 33 | Tyr 52 | Pro 53 | Gly 54 |
| Ser 56 | Tyr 58 | Arg 97 | Ala 99 | Val 100 |
| Tyr 101 | Asp 105 | | | |

Other PDI 42 V_{H} interfacing residues (SARS CoV-2)

| | | | | |
|---|---|---|---|---|
| Gly 416 | Lys 417 | Tyr 453 | Phe 456 | Arg 457 |
| Lys 458 | Ser 459 | Asn 460 | Gly 476 | Ser 477 |
| Phe 486 | Tyr 489 | | | |

SARS CoV-2 RBD and PDI 42 interactions

| | | | | |
|---|---|---|---|---|
| SARS CoV- | | PDI 42 | | Distance |
| 2 RBD | Group | V_{L} | Group | (Å) |

Hydrogen bonds

| | | | | |
|---|---|---|---|---|
| Arg 403 | NH1 | Tyr 92 | O | 3.28 |
| Arg 403 | NH2 | Tyr 92 | O | 3.14 |
| Arg 403 | NH2 | Thr 94 | OG1 | 3.26 |
| Asp 405 | OD2 | Thr 94 | OG1 | 3.42 |
| Tyr 495 | O | Tyr 92 | OH | 2.93 |
| Gly 496 | O | Tyr 92 | OH | 2.97 |
| Asn 501 | ND2 | Ser 28 | O | 3.22 |
| Asn 501 | OD1 | Ser 28 | N | 2.86 |
| Gly 502 | N | Gln 27 | OE1 | 2.98 |

Other SARS CoV-2 interfacing residues (PDI 42 V_{L})

| | | | | |
|---|---|---|---|---|
| Ile 2 | Gln 27 | Ser 28 | Tyr 32 | Gln 55 |
| Tyr 92 | Thr 94 | | | |

Other PDI 42 V_{L} interfacing residues (SARS CoV-2)

| | | | | |
|---|---|---|---|---|
| Gly 416 | Lys 417 | Tyr 453 | Leu 455 | Phe 486 |
| Gln 493 | Gln 498 | Tyr 505 | | |

SARS CoV-2 RBD and PDI 231interactions

| | | | | |
|---|---|---|---|---|
| SARS CoV- | | PDI 231 | | Distance |
| 2 RBD | Group | V_{H} | Group | (Å) |

Hydrogen bonds

| | | | | |
|---|---|---|---|---|
| Lys 417 | NZ | Tyr 52 | OH | 3.7 |
| Asp 420 | OD2 | Ser 56 | OG | 2.5 |
| Tyr 421 | OH | Gly 54 | N | 2.7 |
| Tyr 421 | OH | Ser 53 | N | 3.5 |
| Leu 455 | O | Tyr 33 | OH | 2.4 |
| Lys 458 | O | Ser 53 | OG | 3.4 |
| Asn 460 | ND2 | Gly 54 | O | 3.3 |
| Tyr 473 | OH | Ser 53 | OG | 3 |
| Tyr 473 | OH | Ser 31 | O | 3.3 |
| Ala 475 | O | Asn 32 | ND2 | 2.9 |
| Ala 475 | O | Thr 28 | N | 3.4 |
| Ser 477 | N | Thr 28 | OG1 | 3.8 |
| Glu 484 | OE2 | Ser 103 | OG | 3.9 |
| Tyr 489 | OH | Ser 109 | N | 3.2 |

Other SARS CoV-2 interfacing residues (PDI 231 V_{H})

| | | | | |
|---|---|---|---|---|
| Gly 26 | Leu 27 | Phe 58 | Arg 97 | Tyr 101 |
| Asn 106 | Cys 107 | Val 108 | | |

Other PDI 231 V_{H} interfacing residues (SARS CoV-2)

| | | | | |
|---|---|---|---|---|
| Thr 415 | Gly 416 | Tyr 453 | Phe 456 | Arg 457 |
| Gln 474 | Gly 476 | Gly 485 | Phe 486 | Asn 487 |
| Cys 488 | Gln 493 | | | |

SARS CoV-2 RBD and PDI 231interactions

| | | | | |
|---|---|---|---|---|
| SARS CoV- | | PDI 231 | | Distance |
| 2 RBD | Group | V_{L} | Group | (Å) |

Hydrogen bonds

| | | | | |
|---|---|---|---|---|
| Arg 403 | NH2 | Asn 93 | OD1 | 3.2 |
| Gln 498 | OE1 | Arg 30 | NH2 | 3.3 |
| Asn 501 | OD1 | Arg 30 | NH1 | 2.7 |
| Tyr 505 | OH | Asn 93 | ND2 | 2.8 |
| Tyr 505 | OH | Asp 92 | OD2 | 3.1 |

Salt bridges

| | | | | |
|---|---|---|---|---|
| Arg 403 | NH1 | Asp 92 | OD1 | 3.6 |
| Arg 403 | NH1 | Asp 92 | OD2 | 3.2 |
| Lys 417 | NZ | Asp 92 | OD1 | 3.7 |

Other SARS CoV-2 interfacing residues (PDI 231 V_{L})

| | | |
|---|---|---|
| Asp 28 | Ile 29 | Tyr 32 |

Other PDI 231 V_{L} interfacing residues (SARS CoV-2)

| | | | |
|---|---|---|---|
| Gln 493 | Ser 494 | Gly 496 | Gly 502 |

SARS CoV-2 RBD and WCSL 129 interactions
SARS CoV-2 interfacing residues (WCSL 129 V_{H})

| | | | | |
|---|---|---|---|---|
| Ala 33 | Trp 47 | Ala 50 | Tyr 59 | Val 99 |
| Trp 101 | Gly 102 | | | |

WCSL 129 VH interfacing residues (SARS CoV-2)

| | | | | |
|---|---|---|---|---|
| Leu 455 | Phe 456 | Thr 478 | Phe 486 | Asn 487 |
| Tyr 489 | Gln 493 | | | |

SARS CoV-2 RBD and WCSL 129 interactions

| | | | | |
|---|---|---|---|---|
| SARS CoV- | | WCSL 129 | | Distance |
| 2 RBD | Group | V_{L} | Group | (Å) |

Hydrogen bonds

| | | | | |
|---|---|---|---|---|
| Arg 403 | NH1 | Gln 54 | OE1 | 3.26 |
| Asp 405 | OD2 | Arg 55 | NH2 | 3.6 |
| Lys 417 | N | Asp 53 | OD2 | 2.80 |
| Lys 417 | NZ | Gln 54 | OE1 | 3.15 |
| Tyr 421 | OH | Asn 52 | ND2 | 2.89 |
| Lys 458 | NZ | Ser 31 | O | 2.79 |
| Lys 458 | NZ | Asp 94 | OD1 | 3.35 |
| Tyr 473 | OH | Ser 31 | O | 3.12 |
| Gly 476 | N | Asn 32 | OD1 | 3.41 |
| Ser 477 | N | Asp 93 | O | 3.87 |
| Ser 477 | N | Leu 96 | O | 2.81 |
| Gln 493 | NE2 | Tyr 50 | OH | 3.41 |

Salt bridges

| | | | | |
|---|---|---|---|---|
| Lys 458 | NZ | Asp 94 | OD1 | 3.35 |
| Asp 405 | OD2 | Arg 55 | NH2 | 3.6 |

Other SARS CoV-3 interfacing residues (WCSL 129 V_{L})

| | | | | |
|---|---|---|---|---|
| Pro 33 | Ala 51 | Trp 92 | Ser 95 | Pro 97 |

Other WCSL 129 V_{L} interfacing residues (SARS CoV-2)

| | | | | |
|---|---|---|---|---|
| Gly 416 | Leu 455 | Phe 456 | Ala 475 | Thr 478 |
| Phe 486 | Asn 487 | Tyr 489 | Tyr 505 | |

SARS CoV-2 RBD and PDI 210 interactions

| | | | | |
|---|---|---|---|---|
| SARS CoV- | | PDI 210 | | Distance |
| 2 RBD | Group | V_{H} | Group | (Å) |

Hydrogen bonds

| | | | | |
|---|---|---|---|---|
| Arg 403 | NH1 | Thr 28 | OG1 | 3.5 |
| Tyr 421 | OH | Asn 103 | OD1 | 3.0 |
| Tyr 449 | OH | Ala 73 | O | 3.3 |
| Tyr 453 | OH | Ser 30 | OG | 2.9 |
| Arg 457 | O | Asn 103 | ND2 | 2.6 |
| Arg 457 | N | Asn 103 | OD1 | 3.3 |
| Tyr 489 | OH | Glu 50 | OE2 | 2.7 |
| Phe 490 | O | Tyr 33 | OH | 3.8 |
| Ser 494 | O | Ser 30 | OG | 2.5 |
| Gln 498 | NE2 | Ala 73 | O | 3.0 |
| Gln 498 | NE2 | Ser 74 | O | 3.1 |
| Tyr 505 | OH | Glu 27 | OE1 | 2.4 |

Other SARS CoV-2 interfacing residues (PDI 210 V_{H})

| | | | | |
|---|---|---|---|---|
| Gly 31 | Trp 47 | Leu 53 | Arg 54 | Thr 56 |
| Asn 58 | Val 100 | Val 105 | | |

Other PDI 210 V_{H} interfacing residues (SARS CoV-2)

| | | | | |
|---|---|---|---|---|
| Leu 455 | Phe 456 | Lys 458 | Tyr 473 | Ala 475 |
| Glu 484 | Gly 485 | Phe 486 | Gln 493 | Tyr 495 |
| Gly 496 | Asn 501 | | | |

SARS CoV-2 RBD and PDI 210 interactions

| | | | | |
|---|---|---|---|---|
| SARS CoV- | | PDI 210 | | Distance |
| 2 RBD | Group | V_{L} | Group | (Å) |

Hydrogen bonds

| | | | | |
|---|---|---|---|---|
| Ala 475 | O | Tyr 32 | OH | 3.1 |
| Ser 477 | N | Ser 92 | O | 2.5 |
| Asn 487 | OD1 | Arg 91 | NH1 | 3.5 |
| Asn 487 | ND2 | Arg 91 | O | 3.7 |
| Asn 487 | ND2 | Asn 93 | O | 3.3 |
| Tyr 489 | OH | Arg 91 | NH1 | 3.2 |

Other SARS CoV-2 interfacing residues (PDI 210 V_{L})

| | |
|---|---|
| Trp 94 | Leu 95 |

Other PDI 210 V_{L} interfacing residues (SARS CoV-2)

| | | |
|---|---|---|
| Gly 476 | Thr 478 | Phe 486 |

SARS CoV-2 RBD and WCSL 119 interactions

| | | | | |
|---|---|---|---|---|
| SARS CoV- | | WCSL | | Distance |
| 2 RBD | Group | 119 V_{H} | Group | (Å) |

Hydrogen bonds

| | | | | |
|---|---|---|---|---|
| Glu 484 | O | Tyr 33 | N | 3.2 |
| Glu 484 | N | Gly 31 | O | 3.1 |

Other interfacing residues (WCSL 119 V_{H})

| | | | | |
|---|---|---|---|---|
| Thr 30 | Tyr 32 | His 35 | Ser 99 | Tyr 100 |
| Tyr 101 | | | | |

Other interfacing residues on SARS-CoV-2 with WCSL 119 V_{H}

| | | | | |
|---|---|---|---|---|
| Gly 482 | Val 483 | Gly 485 | Phe 486 | Tyr 489 |

SARS CoV-2 RBD and WCSL 119 interactions

| | | | | |
|---|---|---|---|---|
| SARS CoV- | | WCSL | | Distance |
| 2 RBD | Group | 119 V_{L} | Group | (Å) |

Hydrogen bonds

| | | | | |
|---|---|---|---|---|
| Asn 487 | ND2 | Asn 32 | OD1 | 2.8 |
| Asn 487 | OD1 | Ala 33 | N | 3.8 |
| Tyr 489 | OH | His 35 | NE2 | 2.8 |
| Gln 493 | NE2 | Tyr 50 | OH | 3.1 |
| Gln 493 | NE2 | Leu 55 | O | 3.1 |

Other interfacing residues (WSCL 119 V_{L})

| | | | | |
|---|---|---|---|---|
| Tyr 51 | Leu 54 | Ala 57 | Gly 58 | Trp 92 |
| Pro 99 | | | | |

Other interfacing residues on SARS-CoV-2 with WCSL 119 V_{L}

| | | | | |
|---|---|---|---|---|
| Tyr 449 | Leu 455 | Phe 456 | Ala 475 | Phe 486 |

SARS CoV-2 RBD and PDI 215 interactions

| | | | | |
|---|---|---|---|---|
| SARS CoV- | | PDI 215 | | Distance |
| 2 RBD | Group | V_{H} | Group | (Å) |

Hydrogen bonds

| | | | | |
|---|---|---|---|---|
| Arg 346 | NH1 | Tyr 33 | OH | 3.3 |
| Arg 346 | NH2 | Tyr 33 | OH | 3.3 |
| Arg 346 | NH1 | Tyr 50 | OH | 2.8 |
| Arg 346 | NH2 | Pro 112 | O | 3.1 |
| Ser 349 | N | Ser 56 | O | 3 |
| Ala 352 | O | Ser 56 | OG | 2.8 |
| Asn 354 | ND2 | Asp 110 | O | 2.6 |
| Asn 354 | OD1 | Asp 110 | N | 3 |
| Arg 355 | O | Tyr 109 | N | 2.9 |
| Arg 357 | N | Asp 107 | O | 3.1 |
| Asn 448 | OD1 | Gln 65 | NE2 | 3.8 |
| Tyr 449 | N | Gln 65 | OE1 | 2.7 |
| Asn 450 | ND2 | Asn 59 | OD1 | 3 |
| Asn 450 | N | Gln 65 | OE1 | 3.8 |
| Asn 450 | OD1 | Gln 65 | NE2 | 3.4 |

Salt bridges

| | | | | |
|---|---|---|---|---|
| Glu 471 | OE2 | Lys 76 | NZ | 3.8 |
| Glu 484 | OE1 | Arg 82 | NH1 | 3.1 |
| Glu 484 | OE1 | Arg 19 | NE | 3.4 |
| Glu 484 | OE1 | Arg 82 | NH2 | 3.3 |
| Glu 484 | OE2 | Arg 82 | NH1 | 3.5 |
| Glu 484 | OE2 | Arg 82 | NH2 | 2.5 |

Other SARS CoV-2 interfacing residues (PDI 215V_{H})

| | | | | |
|---|---|---|---|---|
| Gly 54 | Ser 55 | Phe 57 | Thr 58 | Tyr 60 |
| Asp 62 | Gly 66 | Thr 69 | Tyr 71 | Arg 72 |
| Trp 108 | Tyr 114 | Tyr 117 | | |

Other PDI 215 V_{H} interfacing residues (SARS CoV-2)

| | | | | |
|---|---|---|---|---|
| Glu 340 | Val 341 | Ala 344 | Phe 347 | Ala 348 |
| Tyr 351 | Trp 353 | Lys 356 | Lys 444 | Gly 447 |
| Leu 452 | Arg 466 | Ile 468 | Thr 470 | Phe 490 |

SARS CoV-2 RBD and WCSL 129 and PDI 93 interactions
SARS CoV-2 interfacing residues (WCSL 129 V_{H})

| | | | | |
|---|---|---|---|---|
| Ala 33 | Trp 47 | Ala 50 | Tyr 59 | Val 99 |
| Trp 101 | Gly 102 | | | |

WCSL 129 V_{H} interfacing residues (SARS CoV-2)

| | | | | |
|---|---|---|---|---|
| Leu 455 | Phe 456 | Thr 478 | Phe 486 | Asn 487 |
| Tyr 489 | Phe 490 | Leu 492 | Gln 493 | |

SARS CoV-2 RBD and WCSL 129 and PDI 93 interactions

| | | | | |
|---|---|---|---|---|
| SARS CoV- | | WCSL 129 | | Distance |
| 2 RBD | Group | V_{L} | Group | (Å) |

Hydrogen bonds

| | | | | |
|---|---|---|---|---|
| Tyr 473 | OH | Ser 31 | O | 3.5 |
| Gly 476 | N | Asn 32 | OD1 | 3.2 |
| Lys 417 | N | Asp 53 | OD1 | 3.1 |
| Arg 403 | NH1 | Gln 54 | OE1 | 3.4 |
| Lys 417 | NZ | Gln 54 | OE1 | 3.0 |
| Ser 477 | N | Leu 96 | O | 3.0 |
| Gln 493 | OE1 | Tyr 50 | OH | 3.1 |
| Tyr 421 | OH | Asn 52 | ND2 | 3.0 |
| Tyr 421 | OH | Lys 67 | NZ | 3.2 |

Salt bridges

| | | | | |
|---|---|---|---|---|
| Asp 405 | OD2 | Arg 55 | NH2 | 3.7 |

Other SARS CoV-2 interfacing residues (WCSL 129 V_{L})

| | | | | |
|---|---|---|---|---|
| Gly 30 | Pro 33 | Ala 51 | Trp 92 | Asp 93 |
| Asp 94 | Ser 95 | Pro 97 | Pro 99 | |

Other WSCL 129 V_{L} interfacing residues (SARS CoV-2)

| | | | | |
|---|---|---|---|---|
| Tyr 453 | Leu 455 | Phe 456 | Lys 458 | Ala 475 |
| Thr 478 | Phe 486 | Asn 487 | Tyr 489 | Tyr 505 |

| | | | | |
|---|---|---|---|---|
| SARS CoV- | | PDI 93 | | Distance |
| 2 RBD | Group | V_{H} | Group | (Å) |

Hydrogen bonds

| | | | | |
|---|---|---|---|---|
| Tyr 449 | OH | Lys 52 | NZ | 3.3 |
| Asn 450 | OD1 | His 110 | N | 3.1 |
| Tyr 351 | OH | Thr 106 | O | 3.9 |
| Thr 470 | O | Tyr 31 | OH | 3.8 |
| Glu 484 | OE2 | Ser 30 | OG | 2.8 |
| Ser 494 | N | Asp 56 | OD2 | 3.7 |
| Ser 494 | OG | Asp 56 | OD2 | 2.7 |

Salt bridges

| | | | | |
|---|---|---|---|---|
| Arg 346 | NH2 | Glu 109 | OE2 | 2.6 |

Other SARS CoV-2 interfacing residues (PDI 93 V_{H})

| | | | | |
|---|---|---|---|---|
| Trp 33 | Arg 53 | Ser 55 | Ser 105 | Cys 108 |
| Asp 111 | | | | |

Other PDI 93 V_{H} interfacing residues (SARS CoV-2)

| Ser 349 | Ala 352 | Lys 444 | Tyr 449 | Leu 452 |
|---|---|---|---|---|
| Arg 466 | Ile 468 | Ile 472 | Gly 482 | Phe 490 |
| Gln 493 | | | | |
| SARS CoV- | | PDI 93 | | Distance |
| 2 RBD | Group | V_{L} | Group | (Å) |

Hydrogen bonds

| | | | | |
|---|---|---|---|---|
| Arg 346 | NH2 | Ser 31 | O | 3.2 |
| Arg 346 | NH1 | Ser 31 | OG | 2.6 |
| Asn 440 | O | Arg 30 | NE | 3.7 |
| Leu 441 | O | Tyr 32 | OH | 2.7 |
| Ser 443 | O | Tyr 32 | OH | 3.9 |
| Lys 444 | NZ | Ser 91 | O | 2.7 |
| Lys 444 | NZ | Ser 91 | OG | 2.8 |
| Val 445 | O | Thr 94 | N | 2.8 |
| Val 445 | N | Tyr 92 | O | 3.1 |
| Gly 447 | O | Thr 94 | OG1 | 2.7 |
| Asn 448 | ND2 | Tyr 32 | OH | 3.1 |

Other SARS CoV-2 interfacing residues (PDI 93 V_{L})

| | | | |
|---|---|---|---|
| Ala 50 | Ser 52 | Thr 93 | Pro 95 |

Other PDI 93 V_{L} interfacing residues (SARS CoV-2)

| | | | |
|---|---|---|---|
| Thr 345 | Asp 442 | Gly 446 | Asn 450 |

SARS CoV-2 RBD and WCSL 129 and PDI 96 interactions
SARS CoV-2 interfacing residues (WCSL 129 V_{H})

| | | | | |
|---|---|---|---|---|
| Ala 33 | Trp 47 | Ala 50 | Tyr 59 | Val 99 |
| Trp 101 | Gly 102 | | | |

WCSL 129 V_{H} interfacing residues (SARS CoV-2)

| | | | | |
|---|---|---|---|---|
| Leu 455 | Phe 456 | Thr 478 | Phe 486 | Asn 487 |
| Tyr 489 | Gln 493 | | | |

SARS CoV-2 RBD and WCSL 129 and PDI 96 interactions

| | | | | |
|---|---|---|---|---|
| SARS CoV- | | WSCL 129 | | Distance |
| 2 RBD | Group | V_{L} | Group | (Å) |

Hydrogen bonds

| | | | | |
|---|---|---|---|---|
| Arg 403 | NH2 | Gln 54 | OE1 | 3.3 |
| Thr 415 | OG1 | Lys 67 | O | 3.6 |
| Lys 417 | N | Asp 53 | OD2 | 2.9 |
| Lys 417 | NZ | Gln 54 | OE1 | 3.8 |
| Tyr 421 | OH | Asn 52 | ND2 | 2.9 |
| Lys 458 | NZ | Ser 31 | O | 2.8 |
| Lys 458 | NZ | Asn 32 | OD1 | 3.3 |
| Tyr 473 | OH | Ser 31 | O | 3.2 |
| Gly 476 | N | Asn 32 | OD1 | 3.3 |
| Ser 477 | N | Asp 93 | O | 3.8 |
| Ser 477 | N | Leu 96 | O | 2.9 |
| Tyr 505 | OH | Arg 55 | NH1 | 2.9 |

Salt bridges

| | | | | |
|---|---|---|---|---|
| Asp 405 | OD2 | Arg 55 | NH2 | 3.9 |
| Lys 458 | NZ | Asp 94 | OD1 | 4 |

Other SARS CoV-2 interfacing residues (WCSL 129 V_{L})

| | | | | |
|---|---|---|---|---|
| Pro 33 | Ala 51 | Trp 92 | Ser 95 | Pro 97 |
| Pro 99 | | | | |

Other WCSL 129 V_{L} interfacing residues (SARS CoV-2)

| | | | | |
|---|---|---|---|---|
| Gly 416 | Leu 455 | Phe 456 | Ala 475 | Thr 478 |
| Phe 486 | Asn 487 | Tyr 489 | | |

| | | | | |
|---|---|---|---|---|
| SARS CoV- | | PDI 96 | | Distance |
| 2 RBD | Group | V_{H} | Group | (Å) |

Hydrogen bonds

| | | | | |
|---|---|---|---|---|
| Val 445 | O | Asn 52 | ND2 | 3.1 |
| Ser 443 | O | Phe 104 | N | 2.9 |
| Val 445 | N | Tyr 33 | OH | 2.8 |

Other SARS CoV-2 interfacing residues (PDI 96 V_{H})

| | | | | |
|---|---|---|---|---|
| Ile 50 | Gly 57 | Thr 58 | Thr 102 | Ile 103 |
| Trp 105 | | | | |

Other PDI 96 V_{H} interfacing residues (SARS CoV-2)

| Asn 439 | Asn 440 | Leu 441 | Lys 444 | Gly 446 |
|---|---|---|---|---|
| Pro 499 | Thr 500 | | | |
| SARS CoV- | | PDI 96 | | Distance |
| 2 RBD | Group | V_{L} | Group | (Å) |

Hydrogen bonds

| | | | | |
|---|---|---|---|---|
| Asn 440 | OD1 | Ser 33 | OG | 2.7 |
| Asn 440 | ND2 | Tyr 38 | OH | 3 |
| Asn 440 | ND2 | Asn 34 | OD1 | 3.3 |

Other SARS CoV-2 interfacing residues (PDI 96 V_{L})

| | |
|---|---|
| Tyr 31 | Ser 32 |

Other PDI 96 V_{L} interfacing residues (SARS CoV-2)

| | | | |
|---|---|---|---|
| Ser 373 | Trp 436 | Asn 437 | Leu 441 |

SARS CoV-2 RBD and PDI 222 interactions

| | | | | |
|---|---|---|---|---|
| SARS CoV- | | PDI 222 | | Distance |
| 2 RBD | Group | V_{H} | Group | (Å) |

Hydrogen bonds

| | | | | |
|---|---|---|---|---|
| Asp 420 | OD2 | Arg 103 | NH2 | 2.85 |
| Tyr 421 | OH | Arg 103 | NE | 3.13 |
| Asn 460 | OD1 | Arg 103 | NH2 | 3.03 |
| Ala 475 | O | Cys 106 | N | 2.7 |
| Ser 477 | N | Asp 108 | OD1 | 3.78 |
| Asn 487 | ND2 | Tyr 107 | O | 3.1 |
| Gln 493 | NE2 | Ser 55 | O | 3.22 |

Salt bridges

| | | | | |
|---|---|---|---|---|
| Asp 420 | OD2 | Arg 103 | NH2 | 2.85 |

Other SARS CoV-2 interfacing residues (PDI 222 V_{H})

| | | | | |
|---|---|---|---|---|
| Trp 50 | Val 52 | Gly 54 | Asn 57 | Thr 104 |
| Leu 105 | Asp 108 | Phe 110 | | |

Other PDI 222 V_{H} interfacing residues (SARS CoV-2)

| | | | | |
|---|---|---|---|---|
| Leu 455 | Lys 458 | Tyr 473 | Gly 476 | Gly 485 |
| Phe 486 | Tyr 489 | | | |

SARS CoV-2 RBD and PDI 222 interactions

| | | | | |
|---|---|---|---|---|
| SARS CoV- | | PDI 222 | | Distance |
| 2 RBD | Group | V_{L} | Group | (Å) |

Hydrogen bonds

| | | | | |
|---|---|---|---|---|
| Glu 484 | O | Arg 30 | NH1 | 3.34 |
| Asn 487 | N | Tyr 33 | OH | 3.58 |
| Cys 488 | SG | Tyr 33 | OH | 3.07 |

Other PDI 222 V_{L} interfacing residues (SARS CoV-2)

| | |
|---|---|
| Val 483 | Phe 486 |

Extracted and 2×binned particles for WCSL 119 were imported into cryoSPARC 3.1.0, for 2D classification, ab-initio reconstruction and heterogeneous refinement (Punjani et al., 2017). At this point 329780 particles were exported back into Relion 3.1 for re-extraction without binning. Full-scale particles were imported again into cryoSPARC for homogeneous refinement with per-group CTF parameter optimisation yielding a 4.11 Å map.

For PDI 215 2× binned particles were subject to multiple rounds of 2D classification in Relion 3.1 omitting the CTF until the first peak. After re-extraction without binning, the 17464 particles corresponding to Spike bound to PDI 215 were imported to cryoSPARC, subjected to 2D classification and heterogeneous refinement (using the ab-initio generated model). The 11707 best particles were used for a homogeneous refinement with per-group CTF parameter optimisation yielding 8.3 Å map. Local resolution for all maps was determined in Relion 3.1 with the internal local resolution procedure, using half-reconstructions as input maps.

### High Resolution Data Processing for PDI 222

The PDI 222 dataset collected at 1.06 Å pixel size was subjected to the correction of beam-induced motion using MotionCor2 (Zheng et al., 2017), followed by CTF estimation using Gctf (Zhang, 2016). Micrographs with CTF fit resolution below 4 Å were selected for further examination. Template-based particle picking was performed using Gautomatch (http://ww.mrcAmb.camac.uk/kzhang/) using projections from EMDB 23566 low-pass filtered to 20 Å. Particle coordinates were imported into Relion 3.1 (Zivanov et al., 2018). Particle extraction yielded 522,835 particles binned to 220 pixels (2× binning).

Following 2D and 3D classification, 106927 particles were selected for further processing. Particles were reextracted without rescaling (440 pixel box size) and subjected to a 3D reconstruction using C1 symmetry, yielding a 3.58 Å resolution map. These data were further subjected to 2× rounds of Ctf Refinement (beam tilt, trefoil, 4^{th} order aberrations, anisotropic magnification and per-particle defocus and astigmatism) and Bayesian Polishing (Zivanov et al., 2018). The 3D Refinement of “shiny” particles with C3 symmetry yielded a 2.82 Å resolution consensus map. All RBD appeared to be in the “up” position, however, the density of the Fabs was very poor due to the flexibility of RBD domains relative to the rest of the Spike. To overcome this, focused refinement of an individual RBD bound to a variable Fab fragment (vfAb) was performed. First, to roughly overlay all RBD-fAbs, the symmetry was expanded to C1 in Relion. Following symmetry expansion, the signal for 2 RBD-Fabs and the Spike N-terminal domains was subtracted and the remaining Spike with a single RBD domain bound to Fab was 3D refined to a resolution of 2.8 Å. This was followed by another round of signal subtraction leaving only the signal for a single remaining RBD bound to the Fab variable fragment (representing all Spike RBD-vfAb fragments aligned to each other). The 3D refinement with local searches yielded a 3 Å map. To further improve map quality, particles were subjected to 2 rounds of 3D classification without alignments followed by masked 3D refinement with local searches. The final dataset of 182,255 particles yielded a 3.12 Å resolution map based on a gold standard Fourier shell correlation cut-off of 0.143. Local resolution was determined using the internal local resolution procedure in Relion, using half-reconstructions as input maps.

### Model Building

Following focused refinement, a high-resolution model was built using the PDI 222-RBD map. The COVOX 253 RBD crystal structure (PDBID: 7BEN) was used for rigid-body docking of the RBD and vfAb following by iterative model adjustment and rebuilding in COOT (Emsley et al., 2010) and real-space refinement in PHENIX (Adams et al., 2002). Model validation was performed in MolProbity (Chen et al., 2010). Figures were prepared using the PyMOL Molecular Graphics System, Version 2.3.0 (Schrôdinger, LLC) and Chimera v1.1.3 (Pettersen et al., 2021).

### Statistical Analyses

Grouped data are generally presented as median +/−IQR, with groups compared by Mann-Whitney U tests using Prism 9.0 (Graphpad). Pairwise correlations were assessed using Spearmans tests in Prism 9.0 (Graphpad).

### Example 2: Recovery of Potent Human Neutralising Monoclonal Antibodies Against SARS-CoV-2 Spike

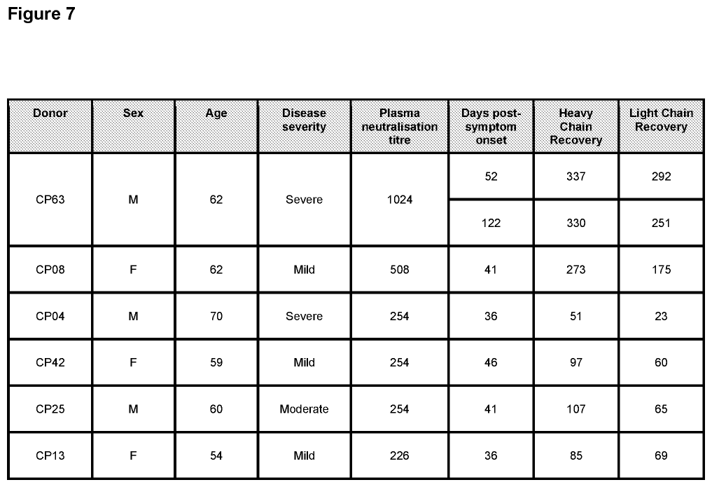

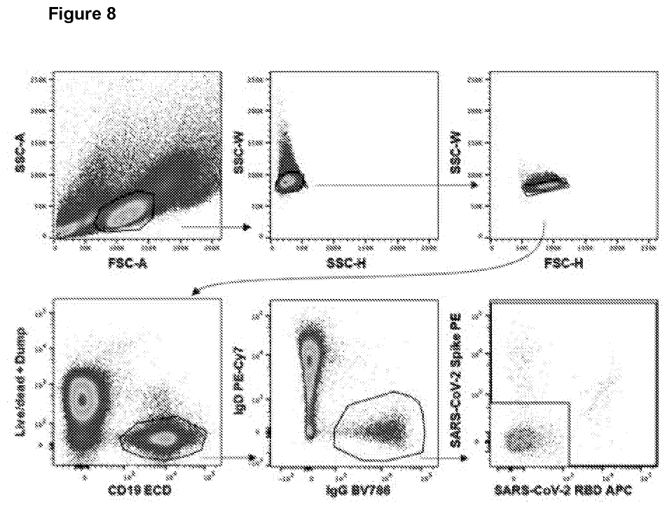

The present inventors have previously described a cohort of individuals recovered from COVID-19 (Juno et al., 2020b) who developed serological binding and neutralising antibodies against SARS-CoV-2 spike following recovery. From 6 donors (FIG. 7), single IgG memory B cells (CD19+IgD−IgG+) that bound to SARS-CoV-2 spike and/or RBD probes were sorted and recovered recombined immunoglobulin gene sequences using multiplex RT-PCR (gating in FIG. 8). A total of 1280 heavy chain immunoglobulins with 935 paired light chains were recovered (FIG. 7), and 212 antibodies (denoted with the prefix PDI) were selected for expression in mammalian cell culture.

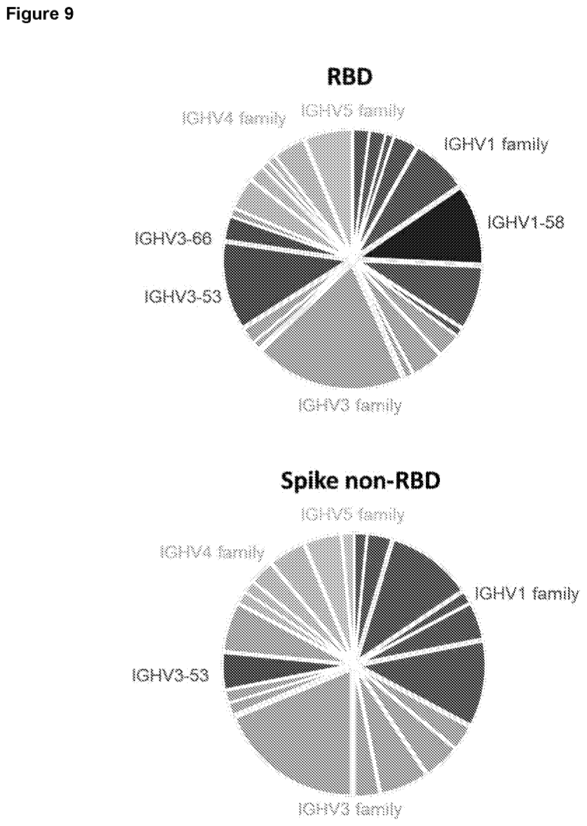

161 of 212 human mAbs expressed bound S or RBD (FIG. 20) and were further screened for neutralising activity against SARS-CoV-2 isolate hCoV-19/Australia/VIC01/2020 using a robust, limiting dilution live-virus microneutralisation format. 69 neutralising mAbs were identified with neutralisation potency ranging from ng/ml to 167 μg/ml (FIG. 1A). Reactivity was assessed by ELISA for binding to SARS-CoV-2 spike (69/69), RBD (56/69) or the NTD (8/69), or for cross-reactive recognition of spike proteins from SARS-CoV-1 (13/69) (FIG. 1B) or endemic human betacoronaviruses HKU1 (0/69) and OC43 (0/69) (data not shown). Sequence analysis of recovered immunoglobulins confirmed recurrent selection of previously described stereotypic antibody classes (FIG. 9), with VH3-53/3-66 (Barnes et al., 2020b; Robbiani et al., 2020; Yan et al., 2021) or VH1-58 (Chen et al., 2021 a; Dejnirattisai et al., 2021) germlines featuring prominently amongst the most potent RBD-specific mAbs. Overall, 26 mAbs showed high potency with an endpoint microneutralisation titre of <2 μg/ml, comparable with well characterised antibodies casirivimab (REGN10933) and imdevimab (REGN10987) (Hansen et al., 2020).

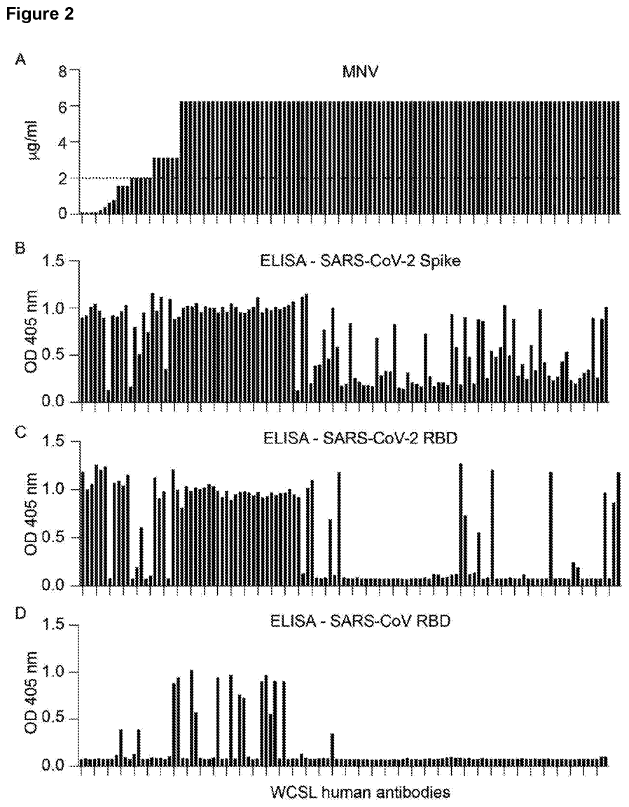

In parallel, a semi-synthetic human Fab library with a diversity of 10¹¹, was screened using SARS-CoV-2 spike or RBD. Of the 760 individual phage clones selected, 121 unique clones were identified and which were then expressed recombinantly (data not shown). All phage display human mAbs (denoted with the prefix WCSL) were also screened for neutralising activity against SARS-CoV-2 virus, with 22 neutralising mAbs identified ranging in neutralisation potency from 0.098 pg/ml to 3.1 μg/ml (FIG. 2A). Reactivity was assessed by ELISA for binding to SARS-CoV-spike (109/121), SARS-CoV-2 RBD (58/121) or SARS-CoV RBD (16/121; FIG. 2B-D). Overall, 16 WCSL mAbs met the criteria demarking high potency with microneutralisation endpoint titre <2 μg/ml).

### Example 3: Structural Definition of the Antigenic Landscape of the RBD

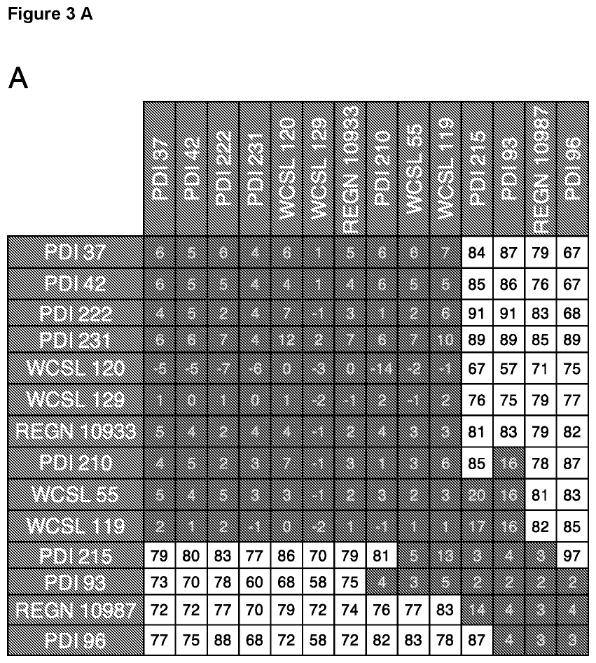

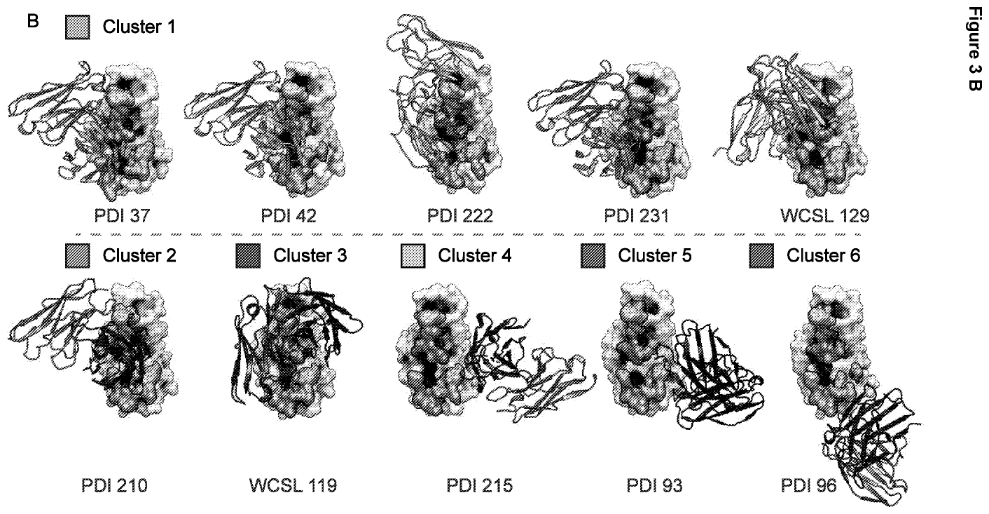

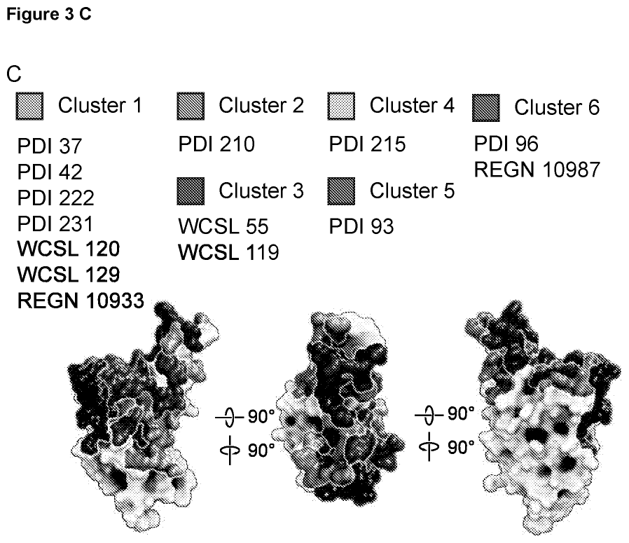

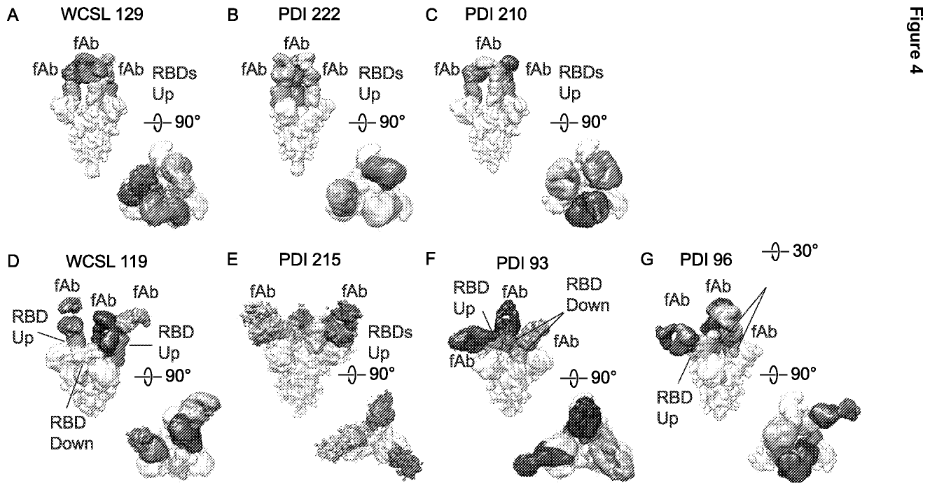

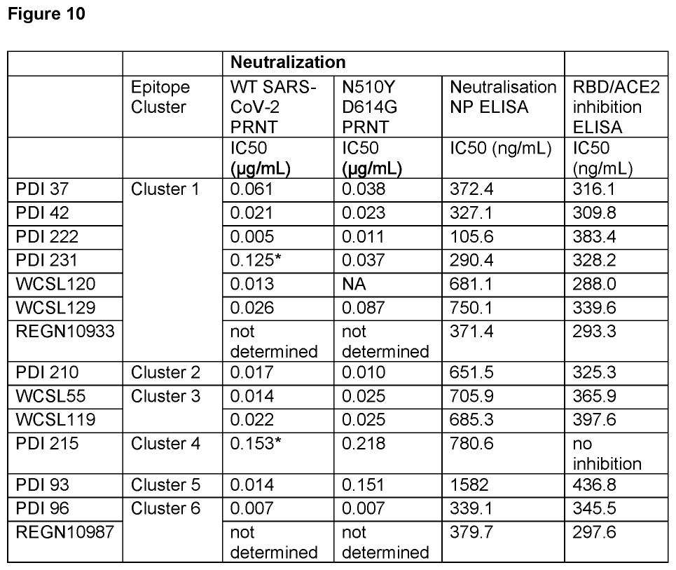

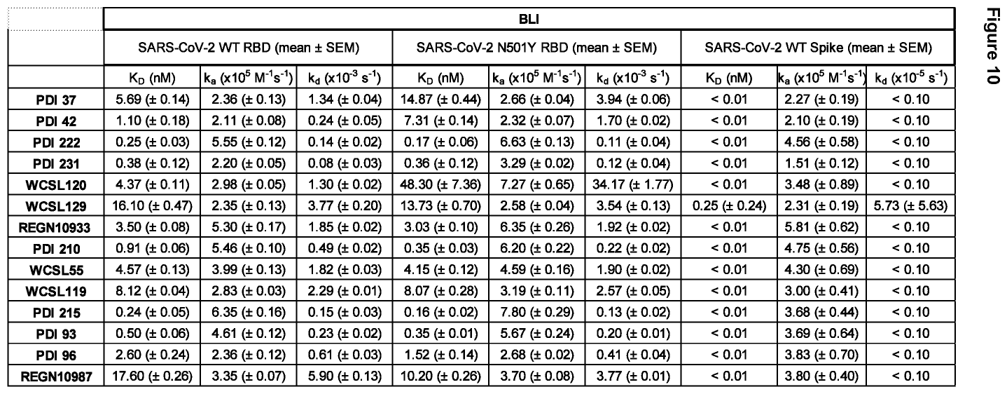

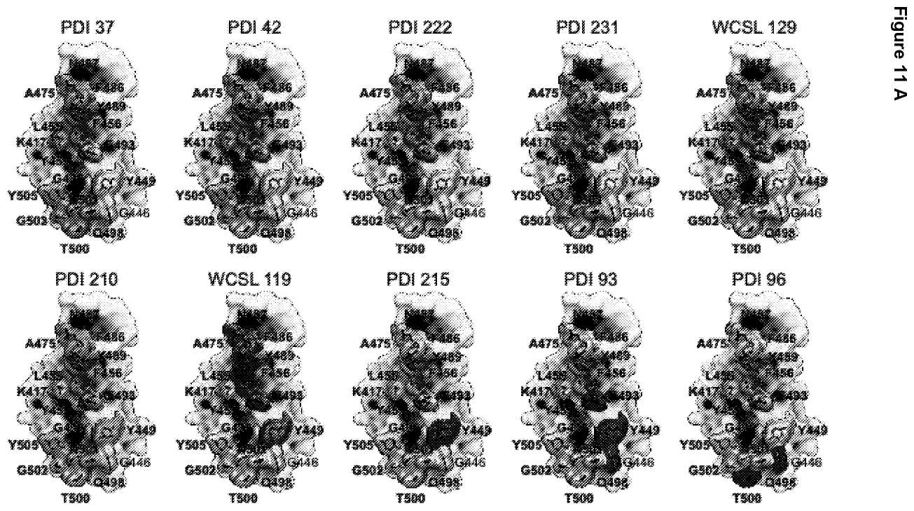

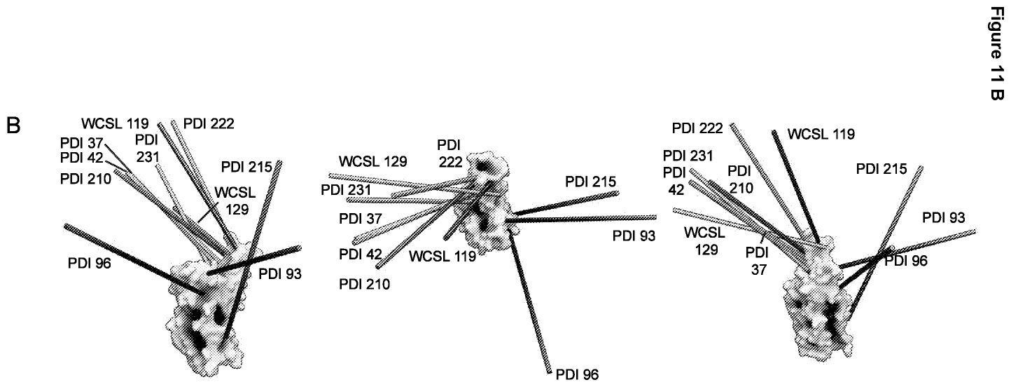

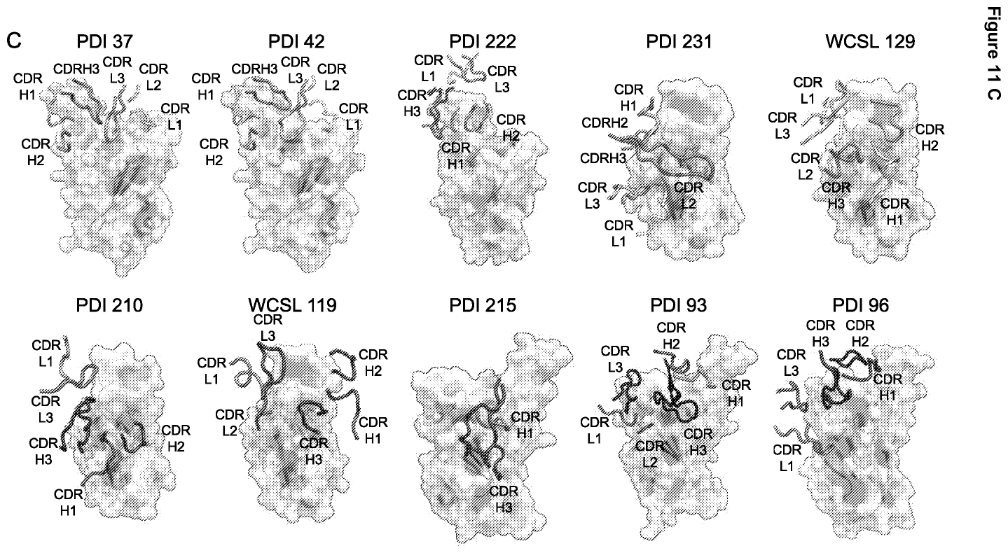

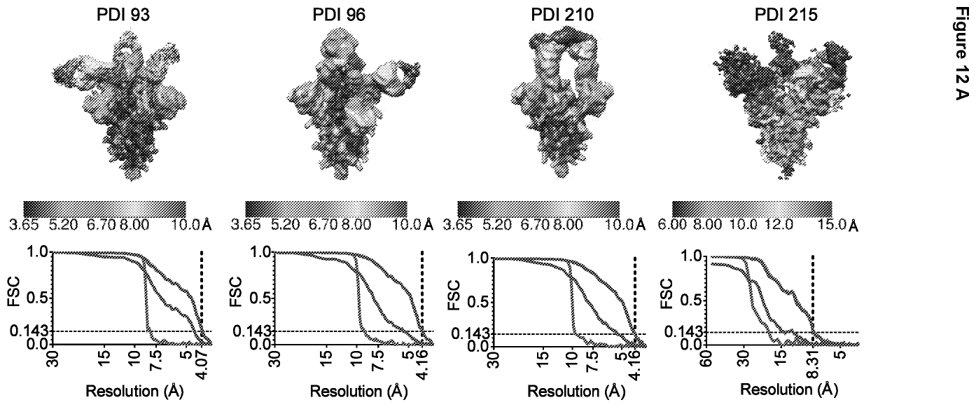

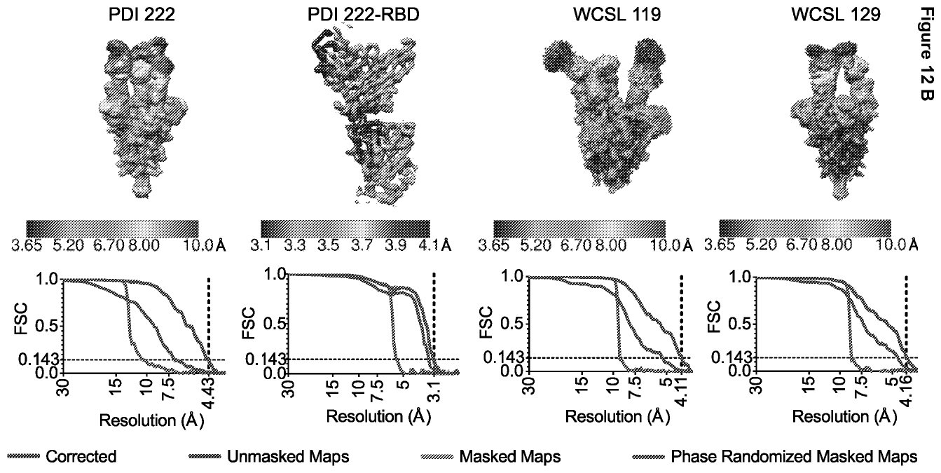

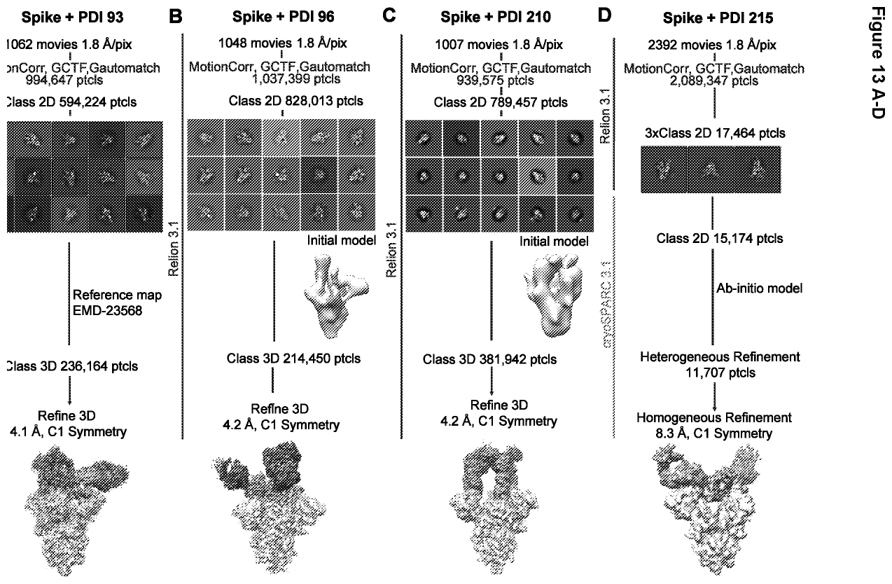

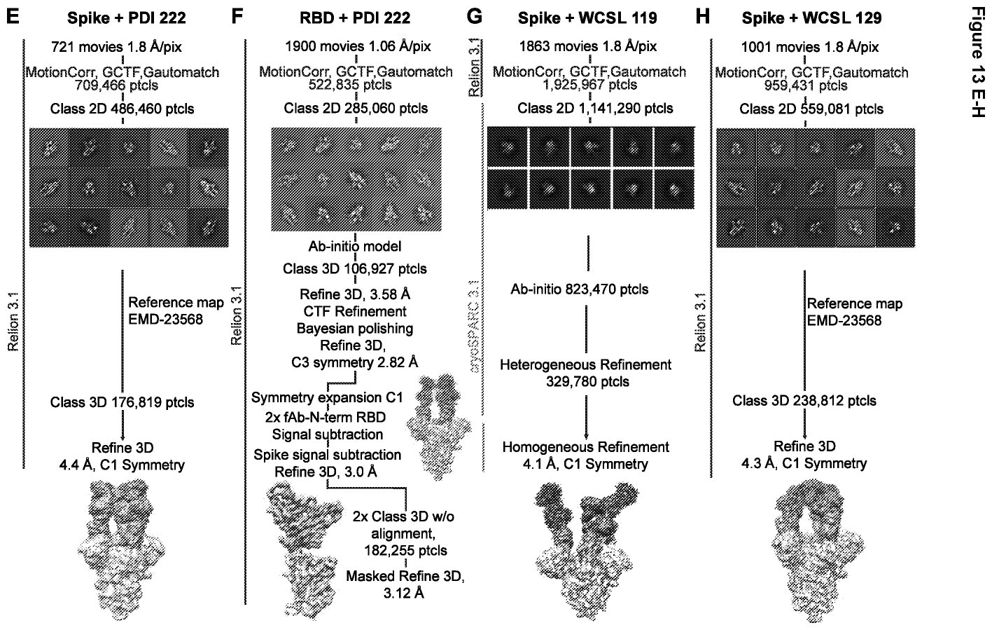

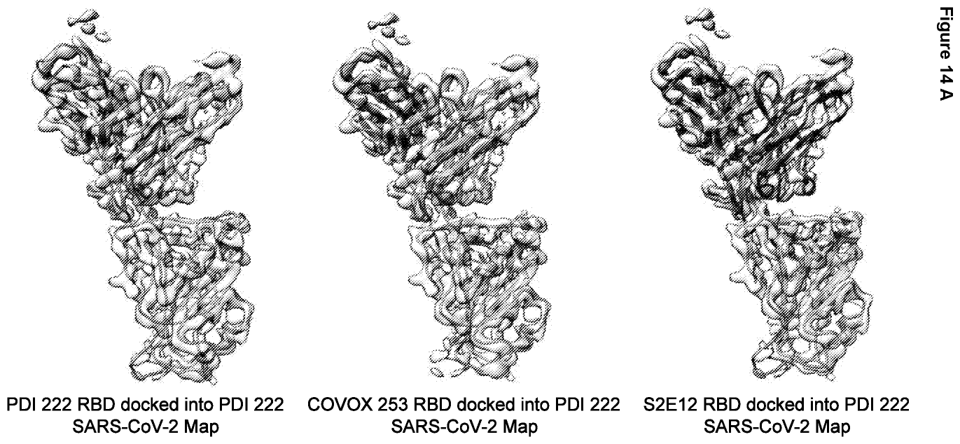

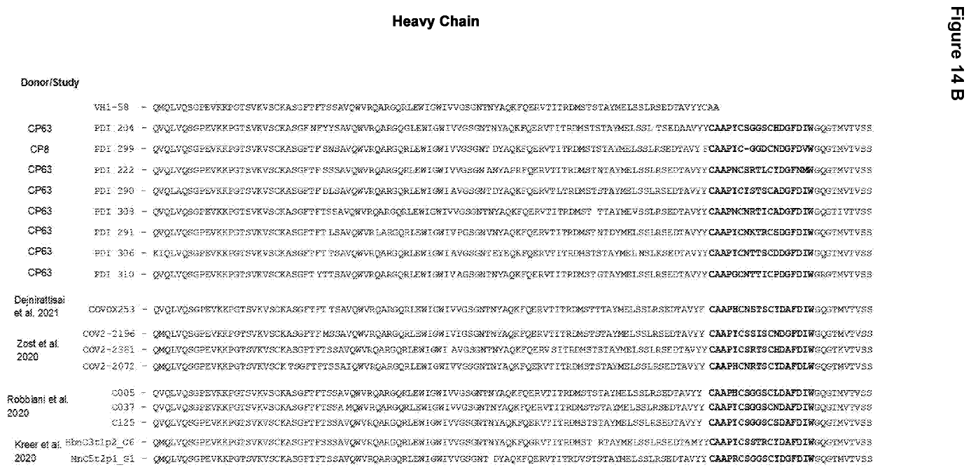

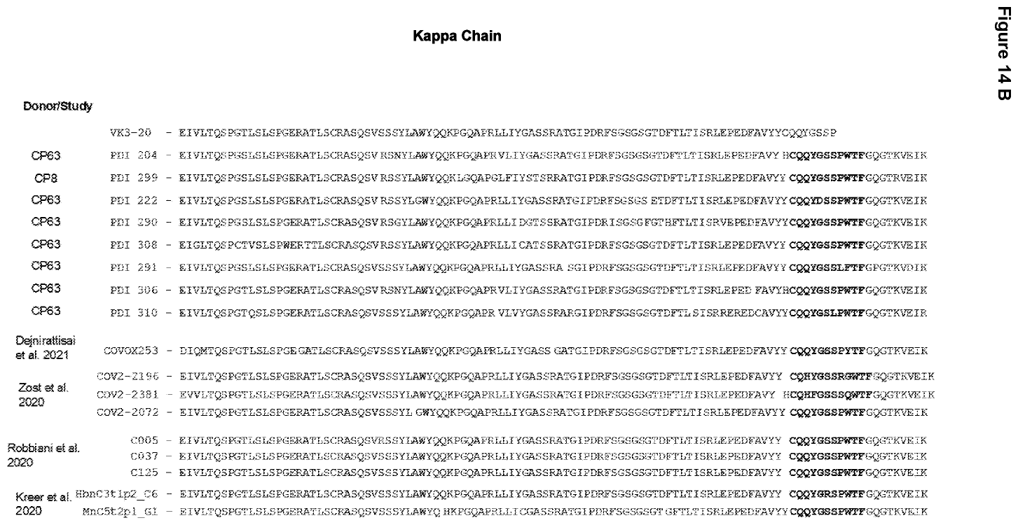

A subset of 12 potently neutralising RBD-specific mAbs representing diverse germline families (8 from convalescent donors and 4 from phage display) were down-selected for further functional and structural analysis. The binding characteristics of each antibody to the RBD was assessed using bio-layer interferometry (BLI), the capacity to block RBD interaction with ACE2 assessed by ELISA, and potent neutralisation activity confirmed using both plaque reduction neutralisation titres (PRNT) and an ELISA-based micro-neutralisation readout (FIG. 10). Epitope diversity was assessed by pairwise competition panning using BLI (FIG. 3A). Based upon clustering patterns of mutual binding inhibition and subsequent structural analysis, six putative epitope clusters distributed widely across the RBD surface were defined (FIG. 3B); cluster 1 (PDI 37, PDI 42, PDI 222, PDI 231, WCSL 120, WCSL 129, REGN10933), cluster 2 (PDI 210), cluster 3 (WCSL 55 and WCSL 119), cluster 4 (PDI 215), cluster 5 (PDI 93) and cluster 6 (PDI 96, REGN10987).

Structural analysis was carried out by X-ray crystallography using co-complex of Fabs bound to monomeric RBD (9 of 12 mAbs) (Table 1 and 2) and by high-resolution cryo-EM using Fab bound to trimeric spike (1 of 12 mAbs) (Table 3). Crystallographic datasets for antibodies were obtained in complex with RBD and both PDI 93 and PDI 96 were obtained in co-complex with RBD and WCSL 129. Antibodies from all clusters displayed partial overlap of binding sites with that of ACE2, providing a mechanistic basis for potent neutralisation activity (FIG. 3B; FIG. 11A). PDI 231, PDI 37, PDI 42 and PDI 210 bound the RBD with similar angles of approach (FIG. B) and had binding footprints which showed the most overlap with the ACE2 interaction site. PDI 231 and PDI 42 contact 14 RBD residues that form part of the ACE2 interaction site (FIG. 11A).

TABLE 3
Cryo-EM data collection, refinement and validation statistics

| | Spike + | | | | | | | |
|---|---|---|---|---|---|---|---|---|
| | PDI 222 | | | | | | | |
| | (high | Spike + | Spike + | Spike + | Spike + | Spike + | Spike + | Spike + |
| | resolution) | PDI 93 | PDI 96 | PDI 210 | PDI 215 | PDI 222 | WCSL 119 | WCSL 129 |
| Data collection | | | | | | | | |
| and processing | | | | | | | | |
| Magnification | 130K | 81K | 81K | 81K | 81K | 81K | 81K | 81K |
| Voltage (kV) | 300 keV | 300 keV | 300 keV | 300 keV | 300 keV | 300 keV | 300 keV | 300 keV |
| Electron exposure | 50 | 50 | 50 | 50 | 52 | 50 | 52 | 50 |
| (e−/Å²) | | | | | | | | |
| Defocus range (μm) | | | | | | | | |
| Pixel size (Å) | 1.06 | 1.8 | 1.8 | 1.8 | 1.8 | 1.8 | 1.8 | 1.8 |
| Symmetry imposed | C1 | C1 | C1 | C1 | C1 | C1 | C1 | C1 |
| Initial particle | 522,835 | 994,647 | 1,037,399 | 939,575 | 2,089,347 | 709,466 | 1,925,957 | 959,431 |
| images (no.) | | | | | | | | |
| Final particle | 182,255 | 236,164 | 214,450 | 381,942 | 11,707 | 176,819 | 329,780 | 238,812 |
| images (no.) | | | | | | | | |
| Map resolution (Å) | 3.12 | 4.1 | 4.2 | 4.2 | 8.3 | 4.4 | 4.1 | 4.3 |
| FSC threshold | 0.143 | 0.143 | 0.143 | 0.143 | 0.143 | 0.143 | 0.143 | 0.143 |
| Map resolution | 3.11-4.12 | 3.7-11.2 | 3.7-12.0 | 3.6-13.7 | 5.8-25 | 3.9-14 | 3.6-16.5 | 3.6-14.6 |
| range (Å) | | | | | | | | |

| | |
|---|---|
| Refinement | |
| Initial model | 7BEN |
| used (PDB code) | |
| Model resolution (Å) | 3.20 |
| FSC threshold | 0.5 |
| Map sharpening | −60 |
| B factor (Å²) | |
| Model composition | |
| Non-hydrogen atoms | 3311 |
| Protein residues | 422 |
| Ligands | 1 |
| B factors (Å²) | |
| Protein (min-max | 59-134 |
| (avr)) | (90) |
| Ligand | 97 |
| R.m.s. deviations | |
| Bond lengths (Å) | 0.004 |
| Bond angles (°) | 0.676 |
| Validation | |
| MolProbity score | 1.72 |
| Clashscore | 8.49 |
| Poor rotamers (%) | 0.00 |
| Ramachandran plot | |
| Favored (%) | 96.15 |
| Allowed (%) | 3.85 |
| Disallowed (%) | 0.00 |

Antibodies derived from germlines VH3-53 or VH3-66 with a short CDR-H3 of 9-12 residues comprise a stereotypic class commonly isolated from individuals after recovery from COVID-19 (Robbiani et al., 2020; Yan et al., 2021). Within cluster 1, mAbs PDI 37 and PDI 42 bound similarly to the previously characterised binding mode (Barnes et al., 2020a) (FIG. 3B) and fall within defined public clonotypes 1 and 2 (Tan et al., 2021). In contrast PDI 231, while sharing VH3-53 gene usage, has a longer CDR-H3 at 16 residues (FIG. 20) that diverges from the public clonotype sequence signatures. The longer CDR-H3 loop is accommodated through an alteration of the conformation of the b-sheet and loop between K45 and G57 which is raised away from the RBD surface. This allows protrusion of the CDR H3 loop across the ACE2 binding surface of the RBD without alteration of the approach angle of the antibody (FIG. 3B; FIG. 11C). A previously reported VH3-53 antibody structure (COVA2-39) with an equivalent length CDR-H3 adopts a different angle of approach to the RBD and the position of the heavy chain is rotated nearly 90 degrees from VH3-53/66 binding mode 1.

While not of the same gene usage WCSL 129 bound a similar epitope largely overlapping that of the VH3-53/66 class, but with its centre shifted slightly towards RBD residue S477 (FIG. 11B). Nevertheless, all of the RBD residue contacts of WCSL 129 fall within the epitope defined by the VH3-53/66 class antibodies with no contacts unique to WCSL 129 (FIG. 11A). PDI 210 in cluster 2 also largely overlaps the epitope of the VH3-53/66 mAbs (FIG. 3C), with a slightly larger heavy chain footprint on the RBD (838.7 Å² compared with 765.6 Å² for PDI 37) contacting additional RBD residues Q498 and Y505 that form part of the RBD-ACE2 interface (FIG. 11A).

PDI 222 is derived from a VH1-58 germline sequence which forms another stereotypic class of antibody isolated from convalescent COVID-19 patients (Chen et al., 2021 a; Dejnirattisai et al., 2021; Dong et al., 2021; Robbiani et al, 2020; Kreer et al., 2020; Tortorici et al., 2020). The VH1-58 class has a well-defined epitope on the RBD centered on RBD residue N487 (FIG. 3B; FIG. 11B) overlapping the ACE2 interaction site at fewer residues (7 vs. 14) than the VH3-53/66 class antibodies PDI 37, PDI 42 and PDI 231 (FIG. 11A).

WCSL 119 in cluster 3 bound an epitope centred around RBD residue E484 (FIG. 11B) which partly overlaps with the ACE2 binding site, sharing 8 RBD contacts (FIG. 3B and FIG. 11A). The CDR-H1 and CDR-H3 and the CDR-L1 and CDR-L3 loops are responsible for most contacts with the RBD (FIG. 11C). VH1-2 heavy chain gene usage is also reported to be enriched in the response to SARS-CoV-2 RBD in convalescent subjects (Robbiani et al., 2020), with structures resolved to date falling into two binding modes (Yuan et al., 2021, Rapp et al., 2021). The WCSL 119 epitope overlaps that of previously reported VH1-2 binding modes though the interactions and approach angle differ.

Interestingly, PDI 215 in cluster 4 bound a unique epitope on the distal face of the RBD (FIG. 3C), a location consistent with an inability to inhibit ACE2 and RBD interaction in vitro despite one shared interaction residue on the RBD Y449 (FIG. 10 and FIG. 11A). The inability to inhibit ACE2 binding places PDI 215 within the “class 3” RBD antibodies (Barnes et al., 2020a). The CDR-H3 loop of PDI 215 stretches down to contact RBD residue V341, over 27 Å from the nearest ACE2 binding residue (FIG. 11C). The interaction of PDI 215 with the RBD was also unusual in that it is driven entirely by the antibody heavy chain (FIG. 11C), with the closest approach of the light chain to the RBD being over 6.5 Å (FIG. 3B).

In agreement with BLI analysis, PDI 93 (cluster 5) and PDI 96 (cluster 6) bound epitopes distal to cluster 1 and 2 mAbs (FIG. 3C; FIG. 11B). These epitopes interact with fewer ACE2 contact residues than the antibodies in clusters 1-3, sharing only three (PDI 93) or two (PDI 96) contacts on the RBD (FIG. 11A). PDI 96 largely overlaps the resolved epitope for REGN10987 (Hansen et al., 2020), though it has a light chain CDR-L1 loop not found in REGN10987 that binds a cleft on the side of the RBD (FIG. 11C). This forms additional hydrogen bond interactions with the RBD N440 sidechain and contacts the L441 and S373 sidechains. The two contacts shared with the ACE2 epitope are at the top of the PDI 96 binding site at RBD residues G446 and T500 (FIG. 3B; FIG. 11A).

The light chain of PDI 93 overlaps the light chain of the previously characterised C135 (Barnes et al., 2020a), though the orientation of the antibody results in an epitope sharing only 4 of 17 contact residues. A recently published antibody COVOX 278 (Liu et al., 2021) contacts a similar epitope to PDI 93, sharing 17 of the 26 PDI 93 RBD contact residues, notably a difference in positioning of the CDR-H3 loop in PDI 93 might allow for greater tolerance of the L452R mutation than is reported for COVOX 278. Neither chain of PDI 93 directly overlaps ACE2, though the heavy chain interacts with the ACE2 contact residues G446, Y449 and Q493 of the RBD (FIG. 11A).

### Example 4: Antibody Recognition in the Context of the Spike Trimer

Fab binding in the context of the trimeric spike was assessed using cryo-EM. Datasets for PDI 93, PDI 96, PDI 210, PDI 215, PDI 222, WCSL 119 and WCSL 129 were collected at a resolution of 4.07-8.3 Å to allow determination of spike conformation and antibody occupancy (Table 2; FIG. 12 and FIG. 13). Consistent with cryo-EM structures for other mAbs overlapping the ACE-2 binding motif and previously grouped as “class 1” RBD antibodies (Barnes et al., 2020a; Brouwer et al., 2020; Dejnirattisai et al., 2021; Rogers et al., 2020), WCSL 129, PDI 210 and PDI 222 bound the RBD only in the up (open) conformation, with three antibodies bound to each spike trimer (FIG. 4A-C). In contrast, WCSL 119 bound the RBD in both up and down conformations with two of the RBDs up and a single RBD down (FIG. 4D). The complex between PDI 215 and SARS-CoV-2 spike appeared unstable on the grids, with only 1% of particles containing intact spike, which may suggest a mode of neutralisation for this antibody. PDI 215 is bound to two RBDs in the up conformation (FIG. 4E), with the third RBD being disordered and limited density visible for much of the RBD and antibody. PDI 93 and PDI 96 which bind to distal RBD epitopes bound at all three RBDs simultaneously in a one-up and two-down conformation (FIG. 4F, G), which is the most prevalent conformation of this spike construct in the absence of antibody (Hsieh et al., 2020). WCSL 119, PDI 93 and PDI 96 fall into the previously proposed “class 2” RBD antibodies (Barnes et al., 2020a) though they have very distinct epitopes on the RBD.

We recovered multiple highly potent antibodies from VH1-58 germlines, which have recently been identified as another stereotypic class in humans (Chen et al., 2021a; Dejnirattisai et al., 2021; Dong et al., 2021). High resolution cryo-EM of the PDI 222-spike complex shows an antibody interaction site on the RBD consistent with the published structures of other VH1-58 class antibodies COVOX-253 (Dejnirattisai et al., 2021) and S2E12 (Tortorici et al., 2020) (FIG. 14). Strong genetic conservation of key determinants such as a di-cysteine motif within the CDR-H3 which is important for the high affinity binding of this clonotype to the SARS-CoV-2 RBD (Dong et al., 2021) and selection of near identical VH3-20-derived kappa chains was observed.

### Example 5: Protective Capacity of Human mAbs in Mice and Hamsters

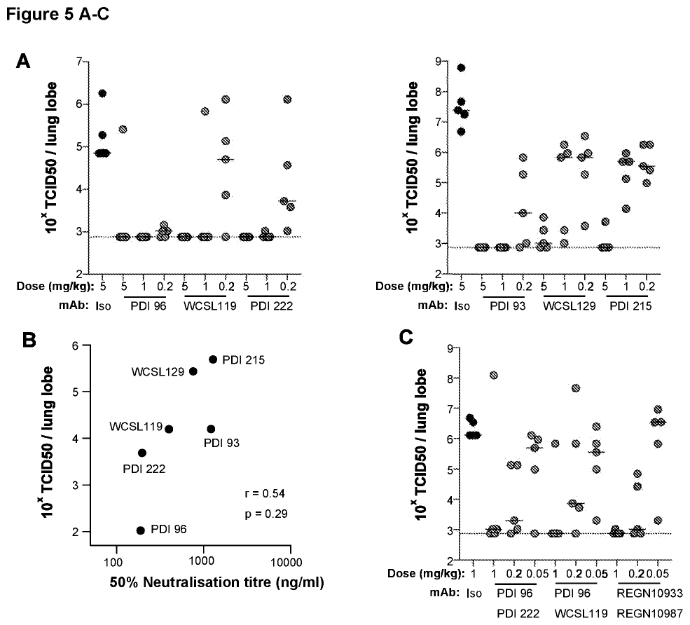

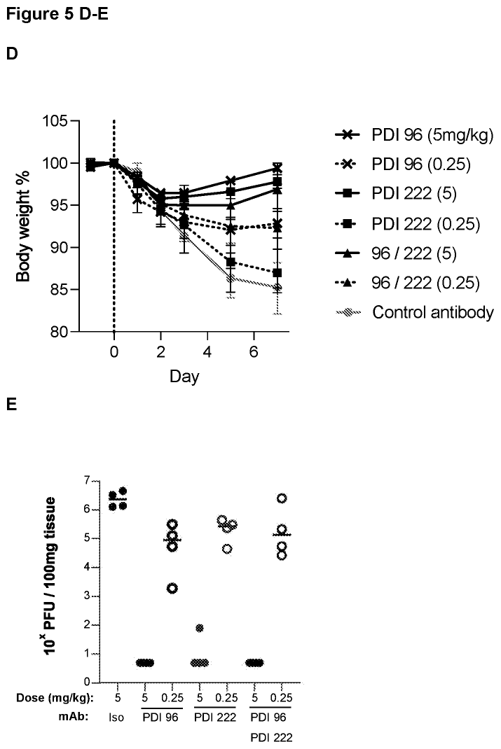

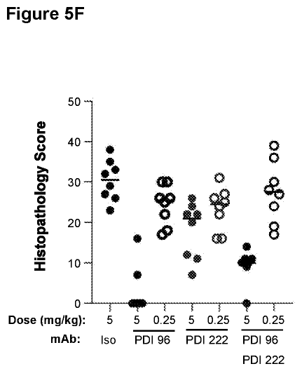

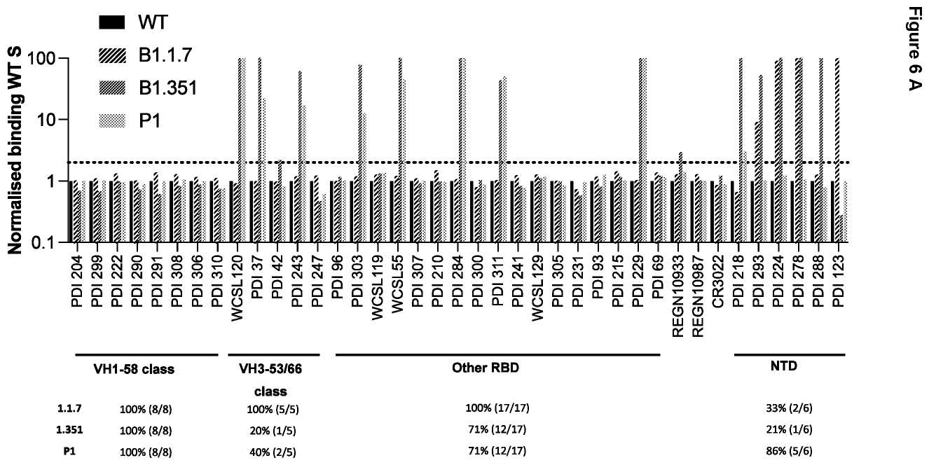

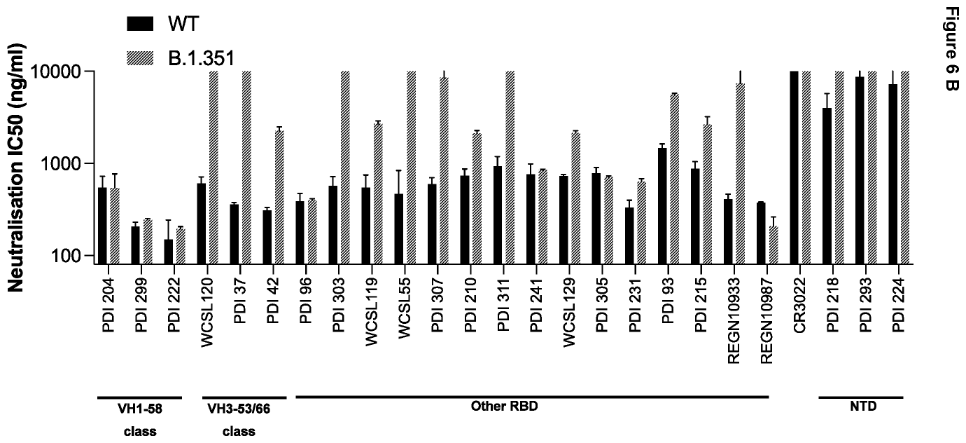

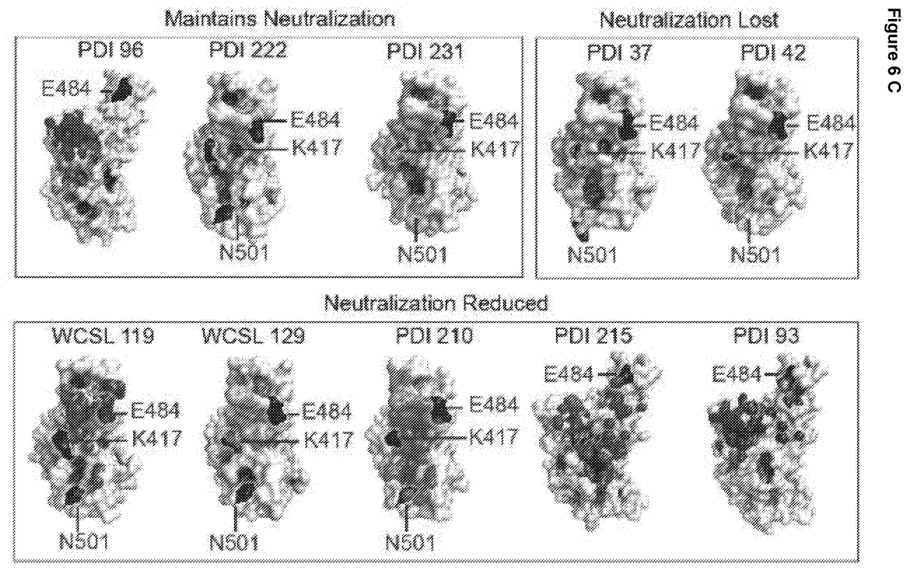

The capacity of mAbs to mediate antiviral activity in vivo was initially assessed using an isolate of SARS-CoV-2 (D614G N501Y) capable of supporting viral replication in wildtype mice (Pymm et al., 2021). Mice were given 5, 1 or 0.2 mg/kg doses of six different mAbs one day prior to aerosolised challenge with SARS-CoV-2, with a primary endpoint of viral titres recoverable within lung homogenates. At 5 mg/kg, significant protection was afforded by all mAbs tested, with protection waning for most mAbs in line with dose (FIG. 5A). Notably, a good correlation existed between in vitro microneutralisation activity and lung viral titres in mice after treatment with the lowest dose of antibody, suggesting neutralisation potency is a key driver of in vivo protective potential (FIG. 5B). Based upon the robust protection observed as single agents, two cocktails consisting of pairs of antibodies binding non-competing RBD epitopes were selected: WCSL 119/PDI 96 (clusters 3 and 6) and PDI 222/PDI 96 (clusters 1 and 6). When used to prophylactically treat mice, both cocktails demonstrated potent protection at 1 mg/kg, comparable to the benchmark Regeneron cocktail, with some protective effects still observable at the low dose of 0.2 mg/kg (FIG. 5C).

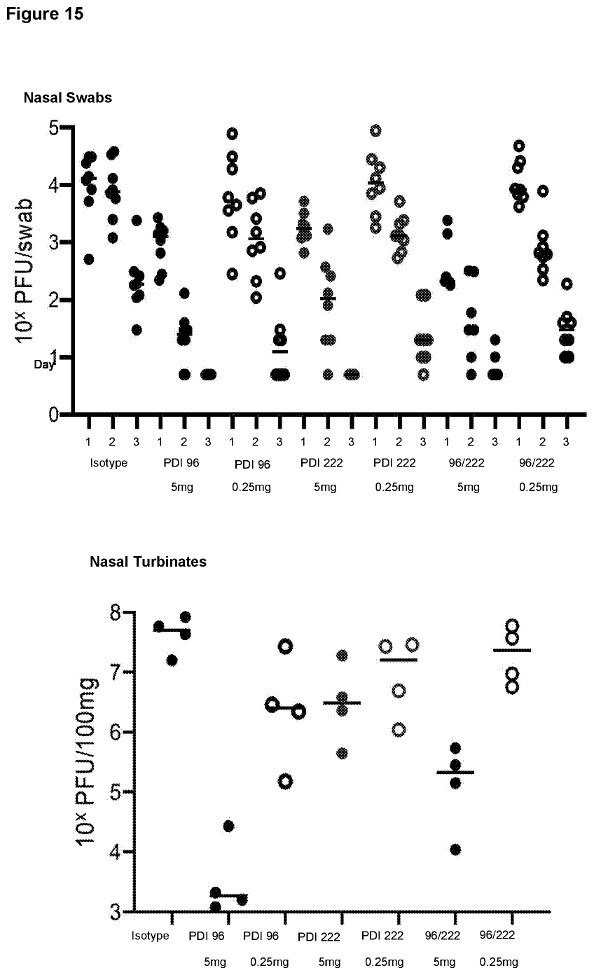

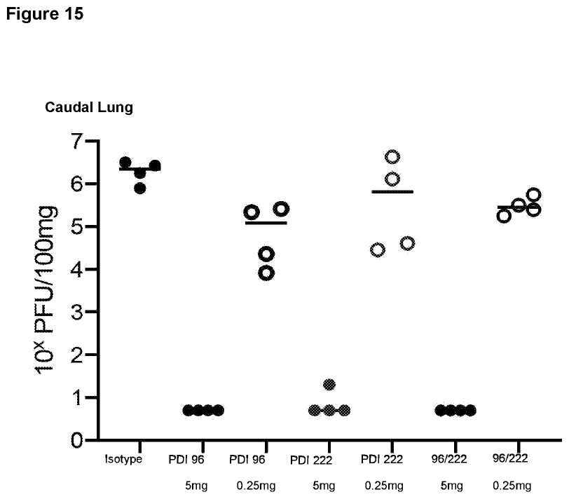

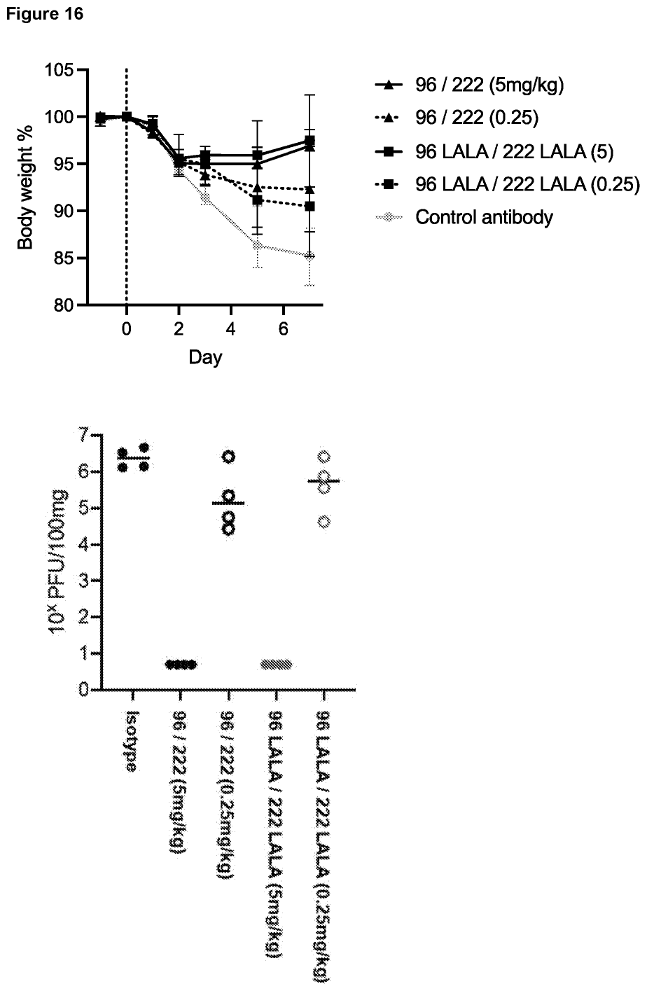

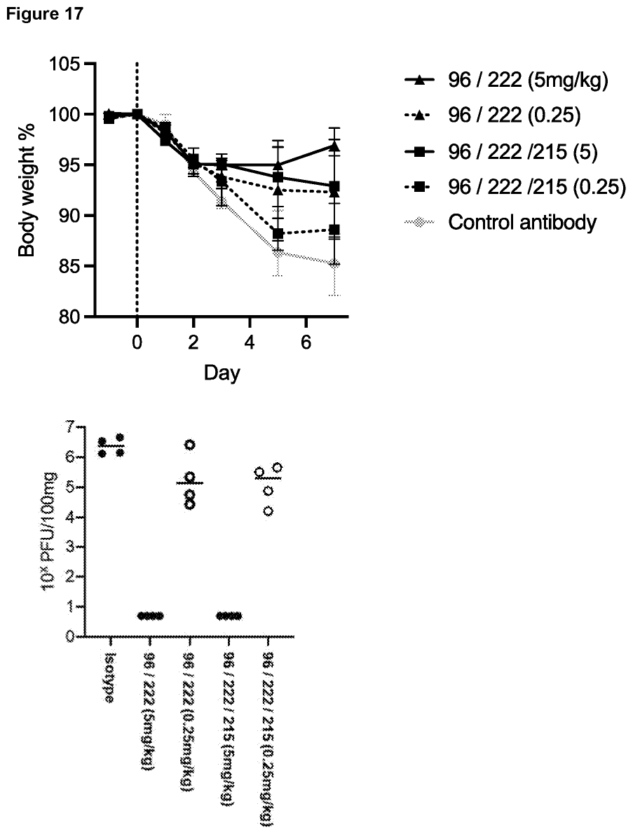

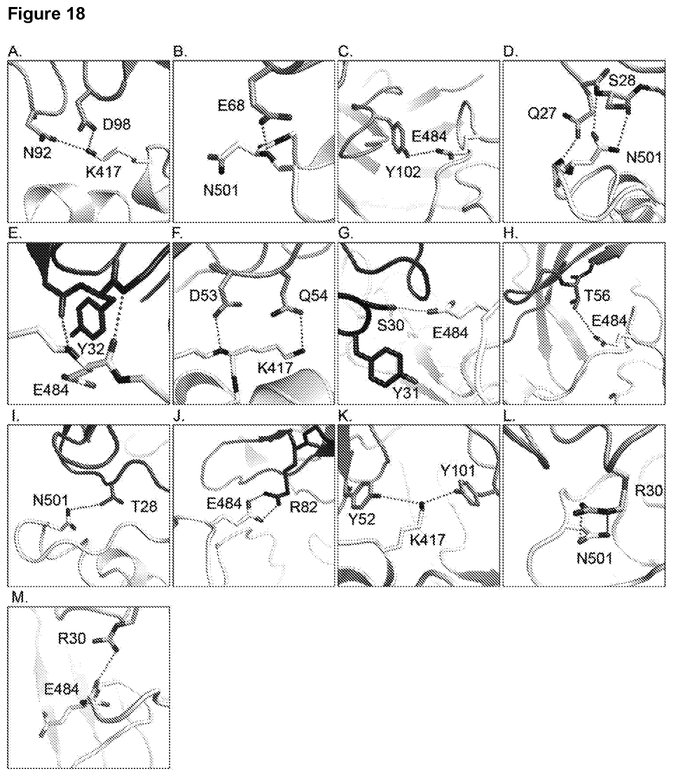

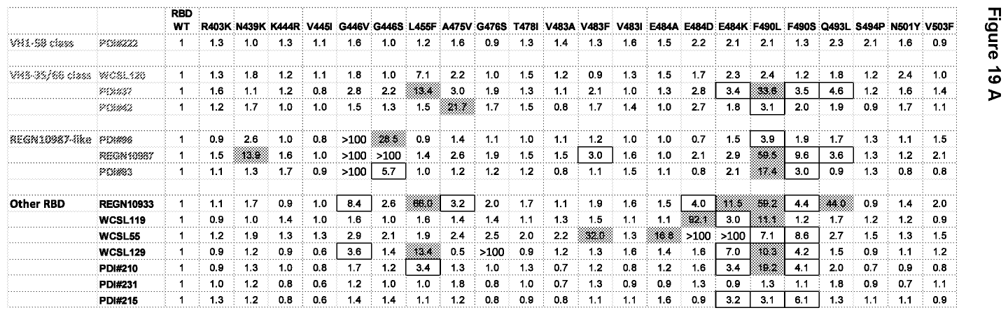

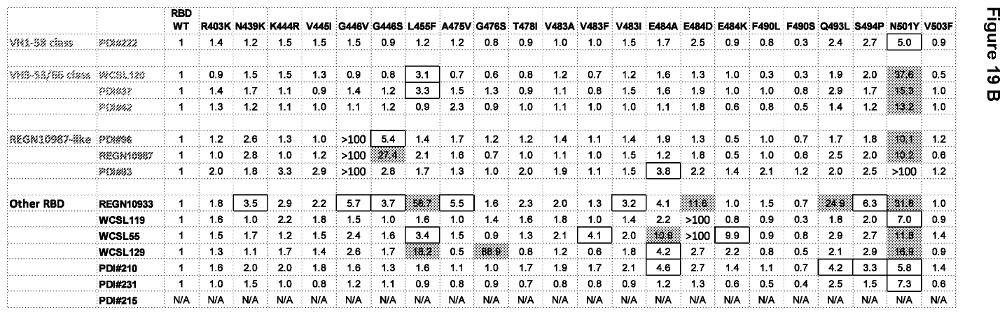

We next studied Syrian hamsters, a model with more human-relevant pathogenesis, to further explore the potential of mAbs as treatments and prophylaxis of COVID-19. Antibodies PDI 222 and PDI 96 were delivered singly or in a cocktail via intraperitoneal injection one day prior to intranasal challenge with SARS-CoV-2 as previously described (Schafer et al., 2021). Passive infusion at 5 mg/kg of each single antibody or a cocktail provided robust protection against SARS-CoV-2-induced weight loss (FIG. 5D), with levels of virus at 3 dpi virtually undetectable in the cranial (FIG. 5E) or the caudal lung (FIG. 15). Virus levels within nasal turbinates (3 dpi) or recovered from swabs (1-3 dpi) were dramatically reduced relative to animals dosed with isotype controls, suggesting accelerated viral clearance after challenge (FIG. 15). Reduced protection was observed at 0.25 mg/kg dosing, however an ˜1.5 log reduction in viral levels in the lung and nasal passage and reduced weight loss were still evident. The contribution of Fc-mediated effector functions was assessed using variants of PDI 96 and PDI 222 modified with inclusion of LALA mutations proven to abrogate Fc-receptor engagement across a wide range of species (Saunders, 2019). Both weight loss and viral levels in the lung were unchanged relative to unmodified antibodies (FIG. 16), suggesting protection is primarily driven by neutralisation potency alone in this model. The protective capacity of a cocktail of three non-competing RBD mAbs (PDI 96, PDI 222 and PDI 215) was compared, however no additional protective benefit from inclusion of a third mAb was observed in this example (FIG. 17).

Gross pathology in the lung was assessed using haemotoxylin and eosin staining on animals sacrificed at 3 and 7 dpi. Infusion with 5 mg/kg PDI 96 or the PDI 96/222 cocktail provided robust protection against lung pathology associated with SARS-CoV-2 challenge (FIG. 5F), with PDI 222 providing moderate protection. Infusion with 0.25 mg/kg of either single or a cocktail of antibodies did not prevent lung pathology.

### Example 6: Epitope Resilience of Human mAbs in the Context of SARS-Cov-2 VOC

The emergence of SARS-CoV-2 variants with reduced susceptibility to neutralisation by antibody is a significant confounder for currently authorised treatments. A panel of neutralising mAbs (N=40) was screened for any loss of recognition to recombinant spike proteins from B.1.351, B.1.1.7 or P.1 VOC. In line with other reports (Chen et al., 2021b; Wang et al., 2021 c), a significant loss of binding to B.1.351 and P.1 VOC was observed for approximately a third of anti-RBD mAbs, most strikingly those from the VH3-53/66 stereotypic class including PDI 37, PDI 42 and WCSL 120 (FIG. 6A). In contrast, binding to B.1.1.7 was largely unaffected. Recognition by NTD-specific neutralising mAbs was compromised for B.1.351 (1 of 6 still binding) and B.1.1.7 (2 of 6) while the recognition of P.1 was largely maintained (5 of 6).

Neutralising activity was assessed using a virus neutralisation assay against WT and B.1.351 virus, with many antibodies displaying a significant loss of neutralisation potency against B.1.351 (FIG. 6B). Near total loss of B.1.351 neutralisation was observed for most of the VH3-53/66 stereotypic class and other selected RBD mAbs including REGN10933. Structural analysis reveals such antibodies directly interact with one or more of positions 417, 484 and 501 of the RBD which are substituted in the B.1.351 variant (FIG. 6C). In the VH3-53/66 class PDI 37 forms hydrogen bonds and stacking interactions with the K417 sidechain and hydrogen bonds with N501 (FIG. 17A, B). Similarly, PDI 42 has stacking interactions against the K417 sidechain and forms hydrogen bonds with both E484 and N501 sidechains (FIG. 17C, D), though these interactions would be lost with B.1.351.

Antibodies WCSL 119, WCSL 129, PDI 93, PDI 210, PDI 231 and PDI 215 directly contact at least one residue mutated in the VOC but suffer only a partial reduction (3-4-fold relative to WT) of neutralisation potency (FIG. 6C). Hydrogen bonds between WCSL 119 and RBD residue E484 are solely with the peptide backbone (FIG. 17E), which may be less impacted by substitution, while WCSL 129 forms hydrogen bonds with K417 (FIG. 17F), though some of these might also be maintained with a K417N substitution. PDI 93 forms a hydrogen bond with residue E484 and PDI 210 forms Van der Waals contacts with RBD residues E484 and N501 (FIG. 17G, H, I). PDI 215 forms a salt bridge to RBD residue E484 (FIG. 17J). PDI 231 makes direct contacts with the K417 and N501 sidechains (FIG. 17K, L), however recognition of the RBD is likely maintained via longer CDR-H3 loop making additional contacts across the top surface of the RBD.

As expected, PDI 96 and REGN10987 binding epitopes distal from the relevant E484K and K417N/T mutations (FIG. 5C), bind VOC and mediate potent neutralisation of B.1.351. The resilience of REGN10987-like antibodies was further tested using a multiplex array of single amino-acid RBD changes observed in circulating viruses, with the glycine at position 446 of the RBD flagged as a critical residue for epitope recognition (FIG. 19).

Finally, all antibodies from the VH1-58 stereotypic class (PDI 222, PDI 209, PDI 204) maintain broad recognition of VOC and potent neutralisation of B.1.351. Structural analysis shows that while antibody PDI 222 as well as published VH1-58 antibodies COVOX-253 and S2E12 all bind a shared epitope near the E484 and K417 residues (FIG. 14), the only interaction is a hydrogen bond to the backbone carbonyl oxygen (FIG. 17M). None of the VH1-58 class antibodies directly contact the sidechains of these residues nor residue N501 which is substituted in B.1.351, B.1.1.7 and P.1.

The ability of neutralising antibodies described herein to neutralise B.1.617.2 (Delta) is examined. COV2-2130 (PDI 222) and REGN10987 (PDI 96) are able to neutralise B.1.617.2 (Delta) (Chen, R. E. et al. In vivo monoclonal antibody efficacy against SARS-CoV-2 variant strains. Nature https://doi.org/10.1038/s41586-021-03720-y (2021)). Without wishing to be bound by theory, the present inventors propose that mAb PDI 96 which binds a REGN10987-like epitope, and PDI 222, a VH1-58 class antibody, which binds the upper RBD, binding a COV2-2130-like epitope. REGN10987 and COV2-2130 are able to neutralise B.1.617.2.

### Example 7: Neutralisation of SARS-CoV-2 VOC, Including Omicron BA.1

A panel of neutralising mAbs (N=28) were screened for neutralising activity against the SARS-CoV-2 variants Beta, Delta and Omicron BA.1 using the robust, limiting dilution live-virus microneutralisation format described above.

FIG. 22 demonstrates many antibodies displayed a significant loss of neutralisation potency against Omicron BA.1. For example, REGN10933 and REGN10987 were demonstrated to not neutralise Omicron BA.1. In contrast, PDI 204, PDI 222, PDI 291 and PDI 306 strongly neutralised Omicron BA.1, and PDI 308 and PDI 307 demonstrated intermediate neutralisation of Omicron BA.1. PDI 204, PDI 299, PDI 96, PDI 222, PDI 241, and PDI 305 strongly neutralised Beta.

### Example 8: Neutralisation of SARS-CoV-2 VOC, Including Omicron BA.1, BA.2 and BA.4

A panel of neutralising mAbs (N=22) were screened for neutralising activity against the SARS-CoV-2 variants Omicron BA.1, Omicron BA.2 and Omicron BA.3 using the robust, limiting dilution live-virus microneutralisation format described above.

FIG. 23 demonstrates many antibodies displayed a significant loss of neutralisation potency against Omicron BA.1, BA.2 and/or BA.4. For example, REGN10933 and REGN10987 were demonstrated to not neutralise Omicron BA.1, BA.2 or BA.4. In contrast, PDI 222, PDI 291, PDI 306 and PDI 204 neutralised Omicron BA.1, PDI 222, PDI 291, PDI 306, PDI 204, PDI 231, and PDI 093 neutralised Omicron BA.2, and PDI 204 and PDI 093 neutralised Omicron BA.4.

### REFERENCES

Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., et al. (2010). PHENIX: a comprehensive Python-based system for macromolecular structure solution. Acta Crystallogr D Biol Crystallogr 66, 213-221.
Andreano, E., Nicastri, E., Paciello, I., Pileri, P., Manganaro, N., Piccini, G., Manenti, A., Pantano, E., Kabanova, A., Troisi, M., et al. (2021). Extremely potent human monoclonal antibodies from COVID-19 convalescent patients. Cell 184, 1821-e1816.
Barnes, C. O., Jette, C. A., Abernathy, M. E., Dam, K. A., Esswein, S. R., Gristick, H. B., Malyutin, A. G., Sharaf, N. G., Huey-Tubman, K. E., Lee, Y. E., et al. (2020a). SARS-CoV-2 neutralizing antibody structures inform therapeutic strategies. Nature 588, 682-687.
Barnes, C. O., West, A. P., Jr., Huey-Tubman, K. E., Hoffmann, M. A. G., Sharaf, N. G., Hoffman, P. R., Koranda, N., Gristick, H. B., Gaebler, C., Muecksch, F., et al. (2020b). Structures of Human Antibodies Bound to SARS-CoV-2 Spike Reveal Common Epitopes and Recurrent Features of Antibodies. Cell 182, 828-842 e816.
Baum, A., Ajithdoss, D., Copin, R., Zhou, A., Lanza, K., Negron, N., Ni, M., Wei, Y., Mohammadi, K., Musser, B., et al. (2020a). REGN-COV2 antibodies prevent and treat SARS-CoV-2 infection in rhesus macaques and hamsters. Science 370, 1110-1115.
Baum, A., Fulton, B. O., Wloga, E., Copin, R., Pascal, K. E., Russo, V., Giordano, S., Lanza, K., Negron, N., Ni, M., et al. (2020b). Antibody cocktail to SARS-CoV-2 spike protein prevents rapid mutational escape seen with individual antibodies. Science 369, 1014-1018.
Brouwer, P. J. M., Caniels, T. G., van der Straten, K., Snitselaar, J. L., Aldon, Y., Bangaru, S., Torres, J. L., Okba, N. M. A., Claireaux, M., Kerster, G., et al. (2020). Potent neutralizing antibodies from COVID-19 patients define multiple targets of vulnerability. Science 369, 643-650.
Cerutti, G., Guo, Y., Zhou, T., Gorman, J., Lee, M., Rapp, M., Reddem, E. R., Yu, J., Bahna, F., Bimela, J., et al. (2021). Potent SARS-CoV-2 neutralizing antibodies directed against spike N-terminal domain target a single supersite. Cell Host Microbe 29, 819-833 e817.
Chen, E. C., Gilchuk, P., Zost, S. J., Suryadevara, N., Winkler, E. S., Cabel, C. R., Binshtein, E., Sutton, R. E., Rodriguez, J., Day, S., et al. (2021a). Convergent antibody responses to the SARS-CoV-2 spike protein in convalescent and vaccinated individuals. bioRxiv.
Chen, R. E., Zhang, X., Case, J. B., Winkler, E. S., Liu, Y., VanBlargan, L. A., Liu, J., Errico, J. M., Xie, X., Suryadevara, N., et al. (2021 b). Resistance of SARS-CoV-variants to neutralization by monoclonal and serum-derived polyclonal antibodies. Nat Med 27, 717-726.
Dejnirattisai, W., Zhou, D., Ginn, H. M., Duyvesteyn, H. M. E., Supasa, P., Case, J. B., Zhao, Y., Walter, T. S., Mentzer, A. J., Liu, C., et al. (2021). The antigenic anatomy of SARS-CoV-2 receptor binding domain. Cell 184, 2183-2200 e2122.
Dong, J., Zost, S. J., Greaney, A. J., Starr, T. N., Dingens, A. S., Chen, E. C., Chen, R. E., Case, J. B., Sutton, R. E., Gilchuk, P., et al. (2021). Genetic and structural basis for recognition of SARS-CoV-2 spike protein by a two-antibody cocktail. bioRxiv.
Emsley, P., Lohkamp, B., Scott, W. G., and Cowtan, K. (2010). Features and development of Coot. Acta Crystallogr D Biol Crystallogr 66, 486-501.
Graham, C., Seow, J., Huettner, I., Khan, H., Kouphou, N., Acors, S., Winstone, H., Pickering, S., Galao, R. P., Dupont, L., et al. (2021). Neutralization potency of monoclonal antibodies recognizing dominant and subdominant epitopes on SARS-CoV-2 Spike is impacted by the B.1.1.7 variant. Immunity.
Greaney, A. J., Loes, A. N., Gentles, L. E., Crawford, K. H. D., Starr, T. N., Malone, K. D., Chu, H. Y., and Bloom, J. D. (2021 a). Antibodies elicited by mRNA-1273 vaccination bind more broadly to the receptor binding domain than do those from SARS-CoV-2 infection. Sci Transl Med 13.
Greaney, A. J., Starr, T. N., Barnes, C. O., Weisblum, Y., Schmidt, F., Caskey, M., Gaebler, C., Cho, A., Agudelo, M., Finkin, S., et al. (2021 b). Mutational escape from the polyclonal antibody response to SARS-CoV-2 infection is largely shaped by a single class of antibodies. bioRxiv.
Greaney, A. J., Starr, T. N., Gilchuk, P., Zost, S. J., Binshtein, E., Loes, A. N., Hilton, S. K., Huddleston, J., Eguia, R., Crawford, K. H. D., et al. (2021c). Complete Mapping of Mutations to the SARS-CoV-2 Spike Receptor-Binding Domain that Escape Antibody Recognition. Cell Host Microbe 29, 44-57 e49.
Hansen, J., Baum, A., Pascal, K. E., Russo, V., Giordano, S., Wloga, E., Fulton, B. O., Yan, Y., Koon, K., Patel, K., et al. (2020). Studies in humanized mice and convalescent humans yield a SARS-CoV-2 antibody cocktail. Science 369,1010-1014.
Hsieh, C. L., Goldsmith, J. A., Schaub, J. M., DiVenere, A. M., Kuo, H. C., Javanmardi, K., Le, K. C., Wrapp, D., Lee, A. G., Liu, Y., et al. (2020). Structure-based design of prefusion-stabilized SARS-CoV-2 spikes. Science 369, 1501-1505.
Jones, B. E., Brown-Augsburger, P. L., Corbett, K. S., Westendorf, K., Davies, J., Cujec, T. P., Wiethoff, C. M., Blackbourne, J. L., Heinz, B. A., Foster, D., et al. (2021). The neutralizing antibody, LY-CoV555, protects against SARS-CoV-2 infection in nonhuman primates. Sci Transl Med 13.
Juno, J. A., Tan, H.-X., Lee, W. S., Reynaldi, A., Kelly, H. G., Wragg, K., Esterbauer, R., Kent, H. E., Batten, C. J., Mordant, F. L., et al. (2020a). Immunogenic profile of SARS-CoV-2 spike in individuals recovered from COVID-19. medRxiv, 2020.2005.2017.20104869.
Juno, J. A., Tan, H. X., Lee, W. S., Reynaldi, A., Kelly, H. G., Wragg, K., Esterbauer, R., Kent, H. E., Batten, C. J., Mordant, F. L., et al. (2020b). Humoral and circulating follicular helper T cell responses in recovered patients with COVID-19. Nat Med 26, 1428-1434.
Kemp, S. A., Collier, D. A., Datir, R., Ferreira, I., Gayed, S., Jahun, A., Hosmillo, M., Rees-Spear, C., Mlcochova, P., Lumb, I. U., et al. (2020). Neutralising antibodies in Spike mediated SARS-CoV-2 adaptation. medRxiv.
Krissinel, E., and Henrick, K. (2007). Inference of macromolecular assemblies from crystalline state. J Mol Biol 372, 774-797.
Liu, L., Wang, P., Nair, M. S., Yu, J., Rapp, M., Wang, Q., Luo, Y., Chan, J. F., Sahi, V., Figueroa, A., et al. (2020). Potent neutralizing antibodies against multiple epitopes on SARS-CoV-2 spike. Nature 584, 450-456.
Liu C., Ginn H. M., Dejnirattisai W., Supasa P., Wang B., Tuekprakhon A., Nutalai R., Zhou D., Mentzer A. J., Zhao Y., Duyvesteyn H. M. E., et al. (2021). Reduced neutralization of SARS-CoV-2 B.1.617 by vaccine and convalescent serum. Cell online ahead of print, doi: 10.1016/j.cell.2021.06.020.
McCallum, M., De Marco, A., Lempp, F. A., Tortorici, M. A., Pinto, D., Walls, A. C., Beltramello, M., Chen, A., Liu, Z., Zatta, F., et al. (2021). N-terminal domain antigenic mapping reveals a site of vulnerability for SARS-CoV-2. Cell.
Misasi, J., and Sullivan, N. J. (2021). Immunotherapeutic strategies to target vulnerabilities in the Ebolavirus glycoprotein. Immunity 54, 412-436.
Nielsen, S. C. A., Yang, F., Jackson, K. J. L., Hoh, R. A., Roltgen, K., Jean, G. H., Stevens, B. A., Lee, J. Y., Rustagi, A., Rogers, A. J., et al. (2020). Human B Cell Clonal Expansion and Convergent Antibody Responses to SARS-CoV-2. Cell Host Microbe 28, 516-525 e515.
Panousis, C., Dhagat, U., Edwards, K. M., Rayzman, V., Hardy, M. P., Braley, H., Gauvreau, G. M., Hercus, T. R., Smith, S., Sehmi, R., et al. (2016). CSL311, a novel, potent, therapeutic monoclonal antibody for the treatment of diseases mediated by the common beta chain of the IL-3, G M-CSF and IL-5 receptors. MAbs 8, 436-453.
Pettersen, E. F., Goddard, T. D., Huang, C. C., Meng, E. C., Couch, G. S., Croll, T. I., Morris, J. H., and Ferrin, T. E. (2021). UCSF ChimeraX: Structure visualization for researchers, educators, and developers. Protein Sci 30, 70-82.
Pymm, P., Adair, A., Chan, L. J., Cooney, J. P., Mordant, F. L., Allison, C. C., Lopez, E., Haycroft, E. R., O'Neill, M. T., Tan, L. L., et al. (2021). Nanobody cocktails potently neutralize SARS-CoV-2 D614G N501Y variant and protect mice. Proc Natl Acad Sci USA 118.
Rapp, M., Guo, Y., Reddem, E. R., Yu, J., Liu, L., Wang, P., Cerutti, G., Katsamba, P., Bimela, J. S., Bahna, F. A., et al. (2021). Modular basis for potent SARS-CoV-2 neutralization by a prevalent VH11-2-derived antibody class. Cell Rep 35, 108950.
Robbiani, D. F., Gaebler, C., Muecksch, F., Lorenzi, J. C. C., Wang, Z., Cho, A., Agudelo, M., Barnes, C. O., Gazumyan, A., Finkin, S., et al. (2020). Convergent antibody responses to SARS-CoV-2 in convalescent individuals. Nature 584, 437-442.
Rocca, A., Biagi, C., Scarpini, S., Dondi, A., Vandini, S., Pierantoni, L., and Lanari, M. (2021). Passive Immunoprophylaxis against Respiratory Syncytial Virus in Children: Where Are We Now?Int J Mol Sci 22.
Rogers, T. F., Zhao, F., Huang, D., Beutler, N., Burns, A., He, W. T., Limbo, O., Smith, C., Song, G., Woehl, J., et al. (2020). Isolation of potent SARS-CoV-2 neutralizing antibodies and protection from disease in a small animal model. Science 369, 956-963.
Saunders, K. O. (2019). Conceptual Approaches to Modulating Antibody Effector Functions and Circulation Half-Life. Front Immunol 10, 1296.
Schafer, A., Muecksch, F., Lorenzi, J. C. C., Leist, S. R., Cipolla, M., Bournazos, S., Schmidt, F., Maison, R. M., Gazumyan, A., Martinez, D. R., et al. (2021). Antibody potency, effector function, and combinations in protection and therapy for SARS-CoV-2 infection in vivo. J Exp Med 218.
Subbarao, K., McAuliffe, J., Vogel, L., Fahle, G., Fischer, S., Tatti, K., Packard, M., Shieh, W. J., Zaki, S., and Murphy, B. (2004). Prior infection and passive transfer of neutralizing antibody prevent replication of severe acute respiratory syndrome coronavirus in the respiratory tract of mice. J Virol 78, 3572-3577.
Tan, T. J. C., Yuan, M., Kuzelka, K., Padron, G. C., Beal, J. R., Chen, X., Wang, Y., Rivera-Cardona, J., Zhu, X., Stadtmueller, B. M., et al. (2021). Sequence signatures of two IGHV3-53/3-66 public clonotypes to SARS-CoV-2 receptor binding domain. bioRxiv.
Taylor, P. C., Adams, A. C., Hufford, M. M., de la Torre, I., Winthrop, K., and Gottlieb, R. L. (2021). Neutralizing monoclonal antibodies for treatment of COVID-19. Nat Rev Immunol.
Tiller, T., Meffre, E., Yurasov, S., Tsuiji, M., Nussenzweig, M. C., and Wardemann, H. (2008). Efficient generation of monoclonal antibodies from single human B cells by single cell R T-PCR and expression vector cloning. J Immunol Methods 329, 112-124.
Tortorici, M. A., Beltramello, M., Lempp, F. A., Pinto, D., Dang, H. V., Rosen, L. E., McCallum, M., Bowen, J., Minola, A., Jaconi, S., et al. (2020). Ultrapotent human antibodies protect against SARS-CoV-2 challenge via multiple mechanisms. Science 370, 950-957.
Voss, W. N., Hou, Y. J., Johnson, N. V., Delidakis, G., Kim, J. E., Javanmardi, K., Horton, A. P., Bartzoka, F., Paresi, C. J., Tanno, Y., et al. (2021). Prevalent, protective, and convergent IgG recognition of SARS-CoV-2 non-RBD spike epitopes. Science 372, 1108-1112.
Wang, C., van Haperen, R., Gutierrez-Alvarez, J., Li, W., Okba, N. M. A., Albulescu, I., Widjaja, I., van Dieren, B., Fernandez-Delgado, R., Sola, I., et al. (2021a). A conserved immunogenic and vulnerable site on the coronavirus spike protein delineated by cross-reactive monoclonal antibodies. Nat Commun 12, 1715.
Wang, P., Casner, R. G., Nair, M. S., Wang, M., Yu, J., Cerutti, G., Liu, L., Kwong, P. D., Huang, Y., Shapiro, L., et al. (2021 b). Increased resistance of SARS-CoV-2 variant P.1 to antibody neutralization. Cell Host Microbe.
Wang, P., Nair, M. S., Liu, L., Iketani, S., Luo, Y., Guo, Y., Wang, M., Yu, J., Zhang, B., Kwong, P. D., et al. (2021c). Antibody resistance of SARS-CoV-2 variants B.1.351 and B.1.1.7. Nature.
Wang, Z., Schmidt, F., Weisblum, Y., Muecksch, F., Barnes, C. O., Finkin, S., Schaefer-Babajew, D., Cipolla, M., Gaebler, C., Lieberman, J. A., et al. (2021d). mRNA vaccine-elicited antibodies to SARS-CoV-2 and circulating variants. Nature 592, 616-622.
Weisblum, Y., Schmidt, F., Zhang, F., DaSilva, J., Poston, D., Lorenzi, J. C., Muecksch, F., Rutkowska, M., Hoffmann, H. H., Michailidis, E., et al. (2020). Escape from neutralizing antibodies by SARS-CoV-2 spike protein variants. Elife 9.
Winkler, E. S., Gilchuk, P., Yu, J., Bailey, A. L., Chen, R. E., Chong, Z., Zost, S. J., Jang, H., Huang, Y., Allen, J. D., et al. (2021). Human neutralizing antibodies against SARS-CoV-2 require intact Fc effector functions for optimal therapeutic protection. Cell 184, 1804-1820 e1816.
Yan, Q., He, P., Huang, X., Luo, K., Zhang, Y., Yi, H., Wang, Q., Li, F., Hou, R., Fan, X., et al. (2021). Germline IGHV3-53-encoded RBD-targeting neutralizing antibodies are commonly present in the antibody repertoires of COVID-19 patients. Emerg Microbes Infect, 1-48.
Yuan M., Huang D., Lee C. D., Wu N. C., Jackson A. M., Zhu X., Liu H., Peng L., Gils M. J., Sanders R. W., et al. (2021) Structural and functional ramifications of antigenic drift in recent SARS-CoV-2 variants. Science, onloine ahead of print. DOI: 10.1126/science.abh 1139.
Zhou, D., Dejnirattisai, W., Supasa, P., Liu, C., Mentzer, A. J., Ginn, H. M., Zhao, Y., Duyvesteyn, H. M. E., Tuekprakhon, A., Nutalai, R., et al. (2021). Evidence of escape of SARS-CoV-2 variant B.1.351 from natural and vaccine-induced sera. Cell.
Zost, S. J., Gilchuk, P., Case, J. B., Binshtein, E., Chen, R. E., Nkolola, J. P., Schafer, A., Reidy, J. X., Trivette, A., Nargi, R. S., et al. (2020a). Potently neutralizing and protective human antibodies against SARS-CoV-2. Nature 584, 443-449.
Zost, S. J., Gilchuk, P., Chen, R. E., Case, J. B., Reidy, J. X., Trivette, A., Nargi, R. S., Sutton, R. E., Suryadevara, N., Chen, E. C., et al. (2020b). Rapid isolation and profiling of a diverse panel of human monoclonal antibodies targeting the SARS-CoV-2 spike protein. Nat Med 26, 1422-1427.

1. An antibody or an antigen-binding fragment thereof that neutralises SARS-CoV-2.

2. An antibody or an antigen-binding fragment thereof that neutralises a SARS-CoV-2 variant selected from the group consisting of B.1.1.7 (Alpha), B.1.351 (Beta), B.1.617.2 (Delta), P.1 (Gamma), B.1.427 (Epsilon), B.1.429 (Epsilon), B.1.525 (Eta), B.1.526 (Iota) B.1.617.1 (Kappa), B.1.617.3, and B.1.1.529.

3. An antibody or an antigen-binding fragment thereof according to claim 1, wherein the antibody or an antigen-binding fragment thereof that neutralises SARS-CoV-2 with an IC50 of less than 2 μg/ml.

4. An antibody or an antigen-binding fragment thereof according to claim 1, wherein the antibody is not REGN10933 and REGN10987.

5. An antibody or an antigen-binding fragment thereof according to claim 1 that binds to epitope cluster 1, 2, 3, 4, 5, or 6 of the SARS-CoV-2 spike receptor-binding domain (RBD).

6.-10. (canceled)

11. An antibody or an antigen-binding fragment thereof according to claim 5, wherein the antibody or an antigen-binding fragment thereof comprises
(a) a HCDR1 of EFIVSRNY (SEQ ID NO: 18), a HCDR2 of IYSGGTT (SEQ ID NO: 44), a HCDR3 of ARDRGDYLFDY (SEQ ID NO: 70), a LCDR1 of QSISSW (SEQ ID NO: 96), a LCDR2 of KAS (SEQ ID NO: 122), and a LCDR3 of QQYNSYFPT (SEQ ID NO: 148);
(b) a HCDR1 of GLTVSSNY (SEQ ID NO: 20), a HCDR2 of FYPGGST (SEQ ID NO: 46), a HCDR3 of ARDAVYYGMDV (SEQ ID NO: 72), a LCDR1 of QSISSY (SEQ ID NO: 98), a LCDR2 of AAS (SEQ ID NO: 124), and a LCDR3 of QESYSTPGLFT (SEQ ID NO: 150);
(c) a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134);
(d) a HCDR1 of GLTVSSNY (SEQ ID NO: 19), a HCDR2 of IYSGGST (SEQ ID NO: 45), a HCDR3 of ARVGGYCSSANCVSDV (SEQ ID NO: 71), a LCDR1 of QDIRNY (SEQ ID NO: 97), a LCDR2 of DAS (SEQ ID NO: 123), and a LCDR3 of QQYDNLPIT (SEQ ID NO: 149);
(e) a HCDR1 of GETLSGYY (SEQ ID NO: 11), a HCDR2 of ISLRGTA (SEQ ID NO: 37), a HCDR3 of VGGVVLDNVVWFDP (SEQ ID NO: 63), a LCDR1 of QSVGTY (SEQ ID NO: 89), a LCDR2 of NAS (SEQ ID NO: 115), and a LCDR3 of QQRSNWLT (SEQ ID NO: 141);
(f) a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154);
(q) a HCDR1 of GFTFSDYY (SEQ ID NO: 24), a HCDR2 of ISGGSSFT (SEQ ID NO: 50), and a HCDR3 of ARSPPTGDSSDWYDSPAYNNYYMDV (SEQ ID NO: 76);
(h) a HCDR1 of GFTFSYAW (SEQ ID NO: 21), a HCDR2 of IKRKSDGGTT (SEQ ID NO: 47), a HCDR3 of TTDLCRSTSCEHDAFDI (SEQ ID NO: 73), a LCDR1 of QSIRSY (SEQ ID NO: 99), a LCDR2 of AAS (SEQ ID NO: 125), and a LCDR3 of QQSYTTPAIT (SEQ ID NO: 151);
(i) a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133);
(i) a HCDR1 of GFNFYYSA (SEQ ID NO: 1), a HCDR2 of IVVGSGNT (SEQ ID NO: 27), a HCDR3 of AAPYCSGGSCHDGFDI (SEQ ID NO: 53), a LCDR1 of QSVRSNY (SEQ ID NO: 79), a LCDR2 of GAS (SEQ ID NO: 105), and a LCDR3 of QQYGSSPWT (SEQ ID NO: 131);
(k) a HCDR1 of GFTFSNSA (SEQ ID NO: 2), a HCDR2 of IVVGSGNT (SEQ ID NO: 28), a HCDR3 of AAPYCGGDCNDGFDV (SEQ ID NO: 54), a LCDR1 of QSVRSSY (SEQ ID NO: 80), a LCDR2 of STS (SEQ ID NO: 106), and a LCDR3 of QQYGSSPWT (SEQ ID NO: 132);
(1) a HCDR1 of GFTFDLSA (SEQ ID NO: 5), a HCDR2 of IAVGSGNT (SEQ ID NO: 31), a HCDR3 of AAPYCYSTSCADGFDI (SEQ ID NO: 57), a LCDR1 of QSVRSGY (SEQ ID NO: 83), a LCDR2 of GTS (SEQ ID NO: 109), and a LCDR3 of QQYGSSPWT (SEQ ID NO: 135);
(m) a HCDR1 of GFTFSNAW (SEQ ID NO: 6), a HCDR2 of IKSKTDGGTT (SEQ ID NO: 32), a HCDR3 of TTDPGWWRIAVAGTNY (SEQ ID NO: 58), a LCDR1 of QSISSY (SEQ ID NO: 84), a LCDR2 of AAS (SEQ ID NO: 110), and a LCDR3 of QQSDSSPPT (SEQ ID NO: 136);
(n) a HCDR1 of GFTFTLSA (SEQ ID NO: 7), a HCDR2 of IVPGSGNV (SEQ ID NO: 33), a HCDR3 of AAPYCNKTRCSDGFDI (SEQ ID NO: 59), a LCDR1 of QSVSSSY (SEQ ID NO: 85), a LCDR2 of GAS (SEQ ID NO: 111), and a LCDR3 of QQYGSSLFT (SEQ ID NO: 137);
(o) a HCDR1 of GFTFTSSA (SEQ ID NO: 8), a HCDR2 of IVVGSGNT (SEQ ID NO: 34), a HCDR3 of AAPNCNRTICADGFDI (SEQ ID NO: 60), a LCDR1 of QSVRSSY (SEQ ID NO: 86), a LCDR2 of ATS (SEQ ID NO: 112), and a LCDR3 of QQYGSSPWT (SEQ ID NO: 138);
(p) a HCDR1 of GFTFTTSA (SEQ ID NO: 9), a HCDR2 of IAVGSGNT (SEQ ID NO: 35), a HCDR3 of AAPYCNTTSCDDGFDI (SEQ ID NO: 61), a LCDR1 of QSVRSNY (SEQ ID NO: 87), a LCDR2 of GAS (SEQ ID NO: 113), and a LCDR3 of QQYGSSPWT (SEQ ID NO: 139);
(q) a HCDR1 of GFIFNRYW (SEQ ID NO: 10), a HCDR2 of IKQDGSEK (SEQ ID NO: 36), a HCDR3 of AADLGILWFGDLRKSEP (SEQ ID NO: 62), a LCDR1 of QGISNS (SEQ ID NO: 88), a LCDR2 of AAS (SEQ ID NO: 114), and a LCDR3 of QEYYSLRT (SEQ ID NO: 140);
(r) a HCDR1 of GFTYTTSA (SEQ ID NO: 12), a HCDR2 of IVAGSGNT (SEQ ID NO: 38), a HCDR3 of AAPGCNTTICPDGFDI (SEQ ID NO: 64), a LCDR1 of QSVSSSY (SEQ ID NO: 90), a LCDR2 of GAS (SEQ ID NO: 116), and a LCDR3 of QQYGSLPWT (SEQ ID NO: 142);
(s) a HCDR1 of GGTFSIYA (SEQ ID NO: 13), a HCDR2 of IIPISGTA (SEQ ID NO: 39), a HCDR3 of ARLGRGDYDSSGYYKVYFDY (SEQ ID NO: 65), a LCDR1 of QNIGNY (SEQ ID NO: 91), a LCDR2 of GAS (SEQ ID NO: 117), and a LCDR3 of QKSYSGPYT (SEQ ID NO: 143);
(t) a HCDR1 of GYRFSNYW (SEQ ID NO: 14), a HCDR2 of IDPSDYYT (SEQ ID NO: 40), a HCDR3 of AKHKFFGELPIRGFDP (SEQ ID NO: 66), a LCDR1 of QSVSGSY (SEQ ID NO: 92), a LCDR2 of GAS (SEQ ID NO: 118), and a LCDR3 of QQYGSSPWT (SEQ ID NO: 144);
(u) a HCDR1 of GYSFTTHW (SEQ ID NO: 15), a HCDR2 of IDPSDSYT (SEQ ID NO: 41), a HCDR3 of ARENFWSVYYTGIDYYMDV (SEQ ID NO: 67), a LCDR1 of QGISNS (SEQ ID NO: 93), a LCDR2 of AAS (SEQ ID NO: 119), and a LCDR3 of QQYYSTPYT (SEQ ID NO: 145);
(v) a HCDR1 of GEPLSGYF (SEQ ID NO: 16), a HCDR2 of ISLRGSA (SEQ ID NO: 42), a HCDR3 of SRGVVLNNVVWFDP (SEQ ID NO: 68), a LCDR1 of QSVGSY (SEQ ID NO: 94), a LCDR2 of GVS (SEQ ID NO: 120), and a LCDR3 of QQRSIWLT (SEQ ID NO: 146);
(w) a HCDR1 of GFTFSTYA (SEQ ID NO: 17), a HCDR2 of ISYDGSTK (SEQ ID NO: 43), a HCDR3 of ARDSEDCSSLSCYLDY (SEQ ID NO: 69), a LCDR1 of QSVLYSSNNKNY (SEQ ID NO: 95), a LCDR2 of WAS (SEQ ID NO: 121), and a LCDR3 of QQYYSTPFT (SEQ ID NO: 147);
(x) a HCDR1 of GFIVSSNY (SEQ ID NO: 22), a HCDR2 of LYPGGST (SEQ ID NO: 48), a HCDR3 of ARNIYDAFDI (SEQ ID NO: 74), a LCDR1 of QGIGSY (SEQ ID NO: 100), a LCDR2 of AAS (SEQ ID NO: 126), and a LCDR3 of QQLNSYPQGA (SEQ ID NO: 152);
(y) a HCDR1 of GFTVSSNY (SEQ ID NO: 25), a HCDR2 of IYSGGST (SEQ ID NO: 51), a HCDR3 of ARLPYGDPA (SEQ ID NO: 77), a LCDR1 of QDIRNY (SEQ ID NO: 103), a LCDR2 of DAS (SEQ ID NO: 129), and a LCDR3 of LQYDNLPLT (SEQ ID NO: 155); or
(z) a HCDR1 of GFTFSSFA (SEQ ID NO: 26), a HCDR2 of ISYDGSTK (SEQ ID NO: 52), a HCDR3 of ARDSEDCSSLSCYLDF (SEQ ID NO: 78), a LCDR1 of QSVLYRSNNKNY (SEQ ID NO: 104), a LCDR2 of WAS (SEQ ID NO: 130), and a LCDR3 of QQYYSTPFT (SEQ ID NO: 156).

12.-38. (canceled)

39. An antibody or an antigen-binding fragment thereof according to claim 1, wherein the antibody or an antigen-binding fragment thereof neutralises more than one SARS-CoV-2 variant selected from the group consisting of B.1.1.7 (Alpha), B.1.351 (Beta), B.1.617.2 (Delta), P.1 (Gamma), B.1.427 (Epsilon), B.1.429 (Epsilon), B.1.525 (Eta), B.1.526 (Iota) B.1.617.1 (Kappa), B.1.617.3, and B.1.1.529 (Omicron).

40. A composition comprising at least one antibody or an antigen-binding fragment thereof according to claim 11 and a pharmaceutically acceptable carrier.

41. A composition comprising a first antibody or an antigen-binding fragment thereof that binds to a first epitope cluster of the SARS-CoV-2 spike RBD and a second antibody or an antigen-binding fragment thereof that binds to a second epitope cluster of the SARS-CoV-2 spike RBD, and a pharmaceutically acceptable carrier.

42. A composition according to claim 41 wherein the first epitope cluster of the SARS-CoV-2 spike RBD and the second epitope cluster of the SARS-CoV-2 spike RBD do not overlap.

43. A composition according to claim 41, wherein the first antibody or an antigen binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133), and the second antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134): or
wherein the first antibody or antigen binding fragment thereof comprises a HCDR1 of GYTFTSYY (SEQ ID NO: 3), a HCDR2 of INPSGGGT (SEQ ID NO: 29), a HCDR3 of AKDRVTIFWGNGMDV (SEQ ID NO: 55), a LCDR1 of QSVLYSSNNKNY (SEQ ID NO: 81), a LCDR2 of WAS (SEQ ID NO: 107), and a LCDR3 of HQYSTTPLT (SEQ ID NO: 133), and the second antibody or an antigen-binding fragment thereof comprises a HCDR1 of GFTFSSSA (SEQ ID NO: 4), a HCDR2 of IVVGSGNA (SEQ ID NO: 30), a HCDR3 of AAPNCSRTLCYDGFNM (SEQ ID NO: 56), a LCDR1 of QSVRSSY (SEQ ID NO: 82), a LCDR2 of GAS (SEQ ID NO: 108), and a LCDR3 of QQYDSSPWT (SEQ ID NO: 134), and further comprising a third antibody or an antigen binding fragment thereof comprising a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154).

44. (canceled)

45. A composition comprising a first antibody or an antigen-binding fragment thereof that binds to an epitope cluster 4 of the SARS-CoV-2 spike RBD and a second antibody or an antigen-binding fragment thereof that binds to an epitope cluster of the SARS-CoV-2 spike RBD overlapping with the ACE2 binding site of the spike protein, and a pharmaceutically acceptable carrier.

46. A composition according to claim 45, wherein the antibody or an antigen-binding fragment thereof that binds to an epitope cluster 4 of the SARS-CoV-2 spike RBD.

47. A composition according to claim 46 wherein the antibody or an antigen-binding fragment thereof that binds to an epitope cluster 4 of the SARS-CoV-2 spike RBD comprises a HCDR1 of GFTFSDYY (SEQ ID NO: 24), a HCDR2 of ISGGSSFT (SEQ ID NO: 50), a HCDR3 of ARSPPTGDSSDWYDSPAYNNYYMDV (SEQ ID NO: 76), a LCDR1 of QSVSSSY (SEQ ID NO: 102), a LCDR2 of GAS (SEQ ID NO: 128), and a LCDR3 of QQYGTSPTVT (SEQ ID NO: 154); or
wherein the antibody or an antigen-binding fragment thereof that binds to an epitope cluster 4 of the SARS-CoV-2 spike RBD comprises a HCDR1 of GFTFSDYY (SEQ ID NO: 24), a HCDR2 of ISGGSSFT (SEQ ID NO: 50), and a HCDR3 of ARSPPTGDSSDWYDSPAYNNYYMDV (SEQ ID NO: 76).

48. (canceled)

49. An isolated polynucleotide comprising a nucleic acid which encodes an antibody or an antigen-binding fragment thereof according to claim 1.

50. A vector comprising the polynucleotide of claim 49.

51. A host cell comprising the polynucleotide of claim 50.

52. A method for prophylaxis or treatment of SARS-CoV-2 infection in a subject comprising administering an effective amount of an antibody or an antigen-binding fragment thereof according to claim 1.

53. A method for prophylaxis or treatment of SARS-CoV-2 infection in a subject comprising administering an effective amount of a composition according to claim 40.

54.-55. (canceled)


SEQUENCE LISTING (292 sequences)

SEQ ID NO: 1 - protein, 8 aa, Homo sapiens
GFNFYYSA

SEQ ID NO: 2 - protein, 8 aa, Homo sapiens
GFTFSNSA

SEQ ID NO: 3 - protein, 8 aa, Homo sapiens
GYTFTSYY

SEQ ID NO: 4 - protein, 8 aa, Homo sapiens
GFTFSSSA

SEQ ID NO: 5 - protein, 8 aa, Homo sapiens
GFTFDLSA

SEQ ID NO: 6 - protein, 8 aa, Homo sapiens
GFTFSNAW

SEQ ID NO: 7 - protein, 8 aa, Homo sapiens
GFTFTLSA

SEQ ID NO: 8 - protein, 8 aa, Homo sapiens
GFTFTSSA

SEQ ID NO: 9 - protein, 8 aa, Homo sapiens
GFTFTTSA

SEQ ID NO: 10 - protein, 8 aa, Homo sapiens
GFIFNRYW

SEQ ID NO: 11 - protein, 8 aa, Homo sapiens
GETLSGYY

SEQ ID NO: 12 - protein, 8 aa, Homo sapiens
GFTYTTSA

SEQ ID NO: 13 - protein, 8 aa, Homo sapiens
GGTFSIYA

SEQ ID NO: 14 - protein, 8 aa, Homo sapiens
GYRFSNYW

SEQ ID NO: 15 - protein, 8 aa, Homo sapiens
GYSFTTHW

SEQ ID NO: 16 - protein, 8 aa, Homo sapiens
GEPLSGYF

SEQ ID NO: 17 - protein, 8 aa, Homo sapiens
GFTFSTYA

SEQ ID NO: 18 - protein, 8 aa, Homo sapiens
EFIVSRNY

SEQ ID NO: 19 - protein, 8 aa, Homo sapiens
GLTVSSNY

SEQ ID NO: 20 - protein, 8 aa, Homo sapiens
GLTVSSNY

SEQ ID NO: 21 - protein, 8 aa, Homo sapiens
GFTFSYAW

SEQ ID NO: 22 - protein, 8 aa, Homo sapiens
GFIVSSNY

SEQ ID NO: 23 - protein, 8 aa, Homo sapiens
GGTFSIYA

SEQ ID NO: 24 - protein, 8 aa, Homo sapiens
GFTFSDYY

SEQ ID NO: 25 - protein, 8 aa, Homo sapiens
GFTVSSNY

SEQ ID NO: 26 - protein, 8 aa, Homo sapiens
GFTFSSFA

SEQ ID NO: 27 - protein, 8 aa, Homo sapiens
IVVGSGNT

SEQ ID NO: 28 - protein, 8 aa, Homo sapiens
IVVGSGNT

SEQ ID NO: 29 - protein, 8 aa, Homo sapiens
INPSGGGT

SEQ ID NO: 30 - protein, 8 aa, Homo sapiens
IVVGSGNA

SEQ ID NO: 31 - protein, 8 aa, Homo sapiens
IAVGSGNT

SEQ ID NO: 32 - protein, 10 aa, Homo sapiens
IKSKTDGGTT

SEQ ID NO: 33 - protein, 8 aa, Homo sapiens
IVPGSGNV

SEQ ID NO: 34 - protein, 8 aa, Homo sapiens
IVVGSGNT

SEQ ID NO: 35 - protein, 8 aa, Homo sapiens
IAVGSGNT

SEQ ID NO: 36 - protein, 8 aa, Homo sapiens
IKQDGSEK

SEQ ID NO: 37 - protein, 7 aa, Homo sapiens
ISLRGTA

SEQ ID NO: 38 - protein, 8 aa, Homo sapiens
IVAGSGNT

SEQ ID NO: 39 - protein, 8 aa, Homo sapiens
IIPISGTA

SEQ ID NO: 40 - protein, 8 aa, Homo sapiens
IDPSDYYT

SEQ ID NO: 41 - protein, 8 aa, Homo sapiens
IDPSDSYT

SEQ ID NO: 42 - protein, 7 aa, Homo sapiens
ISLRGSA

SEQ ID NO: 43 - protein, 8 aa, Homo sapiens
ISYDGSTK

SEQ ID NO: 44 - protein, 7 aa, Homo sapiens
IYSGGTT

SEQ ID NO: 45 - protein, 7 aa, Homo sapiens
IYSGGST

SEQ ID NO: 46 - protein, 7 aa, Homo sapiens
FYPGGST

SEQ ID NO: 47 - protein, 10 aa, Homo sapiens
IKRKSDGGTT

SEQ ID NO: 48 - protein, 7 aa, Homo sapiens
LYPGGST

SEQ ID NO: 49 - protein, 8 aa, Homo sapiens
IIPISGTA

SEQ ID NO: 50 - protein, 8 aa, Homo sapiens
ISGGSSFT

SEQ ID NO: 51 - protein, 7 aa, Homo sapiens
IYSGGST

SEQ ID NO: 52 - protein, 8 aa, Homo sapiens
ISYDGSTK

SEQ ID NO: 53 - protein, 16 aa, Homo sapiens
AAPYCSGGSCHDGFDI

SEQ ID NO: 54 - protein, 15 aa, Homo sapiens
AAPYCGGDCNDGFDV

SEQ ID NO: 55 - protein, 15 aa, Homo sapiens
AKDRVTIFWGNGMDV

SEQ ID NO: 56 - protein, 16 aa, Homo sapiens
AAPNCSRTLCYDGFNM

SEQ ID NO: 57 - protein, 16 aa, Homo sapiens
AAPYCYSTSCADGFDI

SEQ ID NO: 58 - protein, 16 aa, Homo sapiens
TTDPGWWRIAVAGTNY

SEQ ID NO: 59 - protein, 16 aa, Homo sapiens
AAPYCNKTRCSDGFDI

SEQ ID NO: 60 - protein, 16 aa, Homo sapiens
AAPNCNRTICADGFDI

SEQ ID NO: 61 - protein, 16 aa, Homo sapiens
AAPYCNTTSCDDGFDI

SEQ ID NO: 62 - protein, 17 aa, Homo sapiens
AADLGILWFGDLRKSEP

SEQ ID NO: 63 - protein, 14 aa, Homo sapiens
VGGVVLDNVVWFDP

SEQ ID NO: 64 - protein, 16 aa, Homo sapiens
AAPGCNTTICPDGFDI

SEQ ID NO: 65 - protein, 20 aa, Homo sapiens
ARLGRGDYDSSGYYKVYFDY

SEQ ID NO: 66 - protein, 16 aa, Homo sapiens
AKHKFFGELPIRGFDP

SEQ ID NO: 67 - protein, 19 aa, Homo sapiens
ARENFWSVYYTGIDYYMDV

SEQ ID NO: 68 - protein, 14 aa, Homo sapiens
SRGVVLNNVVWFDP

SEQ ID NO: 69 - protein, 16 aa, Homo sapiens
ARDSEDCSSLSCYLDY

SEQ ID NO: 70 - protein, 11 aa, Homo sapiens
ARDRGDYLFDY

SEQ ID NO: 71 - protein, 16 aa, Homo sapiens
ARVGGYCSSANCVSDV

SEQ ID NO: 72 - protein, 11 aa, Homo sapiens
ARDAVYYGMDV

SEQ ID NO: 73 - protein, 17 aa, Homo sapiens
TTDLCRSTSCEHDAFDI

SEQ ID NO: 74 - protein, 10 aa, Homo sapiens
ARNIYDAFDI

SEQ ID NO: 75 - protein, 20 aa, Homo sapiens
ARLGRGDYDSSGYYKVYFDY

SEQ ID NO: 76 - protein, 25 aa, Homo sapiens
ARSPPTGDSSDWYDSPAYNNYYMDV

SEQ ID NO: 77 - protein, 9 aa, Homo sapiens
ARLPYGDPA

SEQ ID NO: 78 - protein, 16 aa, Homo sapiens
ARDSEDCSSLSCYLDF

SEQ ID NO: 79 - protein, 7 aa, Homo sapiens
QSVRSNY

SEQ ID NO: 80 - protein, 7 aa, Homo sapiens
QSVRSSY

SEQ ID NO: 81 - protein, 12 aa, Homo sapiens
QSVLYSSNNKNY

SEQ ID NO: 82 - protein, 7 aa, Homo sapiens
QSVRSSY

SEQ ID NO: 83 - protein, 7 aa, Homo sapiens
QSVRSGY

SEQ ID NO: 84 - protein, 6 aa, Homo sapiens
QSISSY

SEQ ID NO: 85 - protein, 7 aa, Homo sapiens
QSVSSSY

SEQ ID NO: 86 - protein, 7 aa, Homo sapiens
QSVRSSY

SEQ ID NO: 87 - protein, 7 aa, Homo sapiens
QSVRSNY

SEQ ID NO: 88 - protein, 6 aa, Homo sapiens
QGISNS

SEQ ID NO: 89 - protein, 6 aa, Homo sapiens
QSVGTY

SEQ ID NO: 90 - protein, 7 aa, Homo sapiens
QSVSSSY

SEQ ID NO: 91 - protein, 6 aa, Homo sapiens
QNIGNY

SEQ ID NO: 92 - protein, 7 aa, Homo sapiens
QSVSGSY

SEQ ID NO: 93 - protein, 6 aa, Homo sapiens
QGISNS

SEQ ID NO: 94 - protein, 6 aa, Homo sapiens
QSVGSY

SEQ ID NO: 95 - protein, 12 aa, Homo sapiens
QSVLYSSNNKNY

SEQ ID NO: 96 - protein, 6 aa, Homo sapiens
QSISSW

SEQ ID NO: 97 - protein, 6 aa, Homo sapiens
QDIRNY

SEQ ID NO: 98 - protein, 6 aa, Homo sapiens
QSISSY

SEQ ID NO: 99 - protein, 6 aa, Homo sapiens
QSIRSY

SEQ ID NO: 100 - protein, 6 aa, Homo sapiens
QGIGSY

SEQ ID NO: 101 - protein, 6 aa, Homo sapiens
QNIGNY

SEQ ID NO: 102 - protein, 7 aa, Homo sapiens
QSVSSSY

SEQ ID NO: 103 - protein, 6 aa, Homo sapiens
QDIRNY

SEQ ID NO: 104 - protein, 12 aa, Homo sapiens
QSVLYRSNNKNY

SEQ ID NO: 105-130 - skipped (26 entries)

SEQ ID NO: 131 - protein, 9 aa, Homo sapiens
QQYGSSPWT

SEQ ID NO: 132 - protein, 9 aa, Homo sapiens
QQYGSSPWT

SEQ ID NO: 133 - protein, 9 aa, Homo sapiens
HQYSTTPLT

SEQ ID NO: 134 - protein, 9 aa, Homo sapiens
QQYDSSPWT

SEQ ID NO: 135 - protein, 9 aa, Homo sapiens
QQYGSSPWT

SEQ ID NO: 136 - protein, 9 aa, Homo sapiens
QQSDSSPPT

SEQ ID NO: 137 - protein, 9 aa, Homo sapiens
QQYGSSLFT

SEQ ID NO: 138 - protein, 9 aa, Homo sapiens
QQYGSSPWT

SEQ ID NO: 139 - protein, 9 aa, Homo sapiens
QQYGSSPWT

SEQ ID NO: 140 - protein, 8 aa, Homo sapiens
QEYYSLRT

SEQ ID NO: 141 - protein, 8 aa, Homo sapiens
QQRSNWLT

SEQ ID NO: 142 - protein, 9 aa, Homo sapiens
QQYGSLPWT

SEQ ID NO: 143 - protein, 9 aa, Homo sapiens
QKSYSGPYT

SEQ ID NO: 144 - protein, 9 aa, Homo sapiens
QQYGSSPWT

SEQ ID NO: 145 - protein, 9 aa, Homo sapiens
QQYYSTPYT

SEQ ID NO: 146 - protein, 8 aa, Homo sapiens
QQRSIWLT

SEQ ID NO: 147 - protein, 9 aa, Homo sapiens
QQYYSTPFT

SEQ ID NO: 148 - protein, 9 aa, Homo sapiens
QQYNSYFPT

SEQ ID NO: 149 - protein, 9 aa, Homo sapiens
QQYDNLPIT

SEQ ID NO: 150 - protein, 11 aa, Homo sapiens
QESYSTPGLFT

SEQ ID NO: 151 - protein, 10 aa, Homo sapiens
QQSYTTPAIT

SEQ ID NO: 152 - protein, 10 aa, Homo sapiens
QQLNSYPQGA

SEQ ID NO: 153 - protein, 9 aa, Homo sapiens
QKSYSGPYT

SEQ ID NO: 154 - protein, 10 aa, Homo sapiens
QQYGTSPTVT

SEQ ID NO: 155 - protein, 9 aa, Homo sapiens
LQYDNLPLT

SEQ ID NO: 156 - protein, 9 aa, Homo sapiens
QQYYSTPFT

SEQ ID NO: 157 - protein, 123 aa, Homo sapiens
QVQLVQSGPEVKKPGTSVKVSCKASGFTFTSSAVQWVRQARGQRLEWIGWIVVGSGNTEYAQKFQERVTITRDMSTSTAYMELSSLRSEDTAVYYCAANNCGRTTCSDAFDIWGQGTMVTLSS

SEQ ID NO: 158 - protein, 130 aa, Homo sapiens
LVQLVQSGAEVKKPGSSVKVSCKASGGTFNSYAISWVRQAPGQGLEWMGGIIPIFATANYAQKFQGRVTITADDSTSTAYMELSSLRSEDTAVYYCARVGAPIERTSSSWHYYYYGMDVWGQGTTVTVSS

SEQ ID NO: 159 - protein, 117 aa, Homo sapiens
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDQGEDVFDIWGQGTMVTVSS

SEQ ID NO: 160 - protein, 118 aa, Homo sapiens
QVQLVQSGAEVKKPGSSVKVSCKASGDTFSNYGINWVRQAPGHGLEWMGGIIPLFATANYAQNFQGRVTITADESTSTAYMELSSLRSEDTAVYYCASFGGDDGLDKWGQGTLVIVSS

SEQ ID NO: 161 - protein, 124 aa, Homo sapiens
QVQLVQSGAEVKKPGASVKVSCKVSGYTLIELSMHWVRQAPGKGLEWMGGFDPEDGETIYAQKFQGRVTMTEDTSTDTAYMELSSLRSEDTAVYYCTTQGSGPFGEPRGWFDPWGQGTLVTVSS

SEQ ID NO: 162 - protein, 117 aa, Homo sapiens
EVQLVESGGGLMQPGGSLRLSCAASGITVSRNYMSWVRQAPGKGLEWVSVMYSGGSTYYADSVKGRFTISRDNSKNTLYLQMNSLRAEDTAVYYCARDLDYYGMDVWGQGTTVTVSS

SEQ ID NO: 163 - protein, 139 aa, Homo sapiens
QVQLVQSGAEVKKPGASVKVSCKVSGYTLTELSLHWVRQAPGKGLEWMGGFDPEDAETIYAQKFQGRVTMTEDTSTDTAYMELSSLTSEDTAVYYCATLAYCTNGVCYILGPPPLRKHEELYYFYMDVWGKGTTVTVSS

SEQ ID NO: 164 - protein, 131 aa, Homo sapiens
LVQLVQSGAEVKKPGASVKVSCKVSGYTLTELSMHWVRQAPGKGLEWMGGFDPEDVETIYAQKFQGRVTMTEDTSTDTAYMELSSLRSEDTAVYFCAASFAVSGTPGPPSHYNYYYGMDVWGQGTTVTVSS

SEQ ID NO: 165 - protein, 131 aa, Homo sapiens
QVQLVQSGAEVKKPGSSAKVSCKASGGSFSRYAVSWVRQAPGQGLEWMGGIIPIFGTPNYAQKFQGRVTITADESTSTAYMELSSLRSEDTAVYYCARTYSTYPKNRLDWDYYYYYGMDVWGQGTTVTVSS

SEQ ID NO: 166 - protein, 128 aa, Homo sapiens
QVQLVQSGAEVKKPGASVKVSCKVSGYTLTELSMHWVRQAPGKGLEWMGGFDPEDAETIYAQKFQGRVTMTEDTSTDTAYMELSSLRSEDTAVYYCATGWSITGTPSNYKYYYGMDVWGQGTTVTVSS

SEQ ID NO: 167 - protein, 117 aa, Homo sapiens
DVQLVETGGGLIQPGGSLRLSCAASGFTVSSNYMSWVRQAPGKGLEWVSVIYSGGSTYYADSVKGRFTISRDNSKNTLYLQMNSLRAEDTAVYYCARDYGDFYFDYWGQGTLVTVSS

SEQ ID NO: 168 - protein, 118 aa, Homo sapiens
EVQLVESGGGVVQPGRSLRLSCAASGFTFSNYAMHWVRQAPGKGLEWVAVISYDGSNKFYADSVKGRFTISRDNSKNTLYLQINSLRAEDTAVYYCAREELRALADYWGQGTLVTVSS

SEQ ID NO: 169 - protein, 120 aa, Homo sapiens
EVQLVESGGGLVKPGGSLRLSCVGSEFTFSSYSMNWVRQAPGKGLEWVSSISTSSSDIYYADAVKGRFTISRDNAKNSLYLEMNSLRVEDTAVYYCASILEWFAGYMDVWGKGTTVTVSS

SEQ ID NO: 170 - protein, 121 aa, Homo sapiens
EVQLVESGGGLIQPGGSLRLSCAASGFTVSSNYMSWVRQAPGKGLEWVSVIYSDGRTHYADSVKGRFTISRDNSKNTLFLQMNRLRAEDTAVFYCASPGIVPAAMGVFDYWGQGTLVTVSS

SEQ ID NO: 171 - protein, 117 aa, Homo sapiens
EVQLVESGGGLIQPGGSLRLSCAASGFTVSSNYMSWVRQAPGKGLEWVSLIYSGGSTDYADSVKGRFTISRDNSKNTLYLQMNSLRAEDTAVYYCARDLVARGMDVWGQGTTVTVSS

SEQ ID NO: 172 - protein, 121 aa, Homo sapiens
QVQLQESGPGLVKPSQTLSLTCTVSGGSISSGGYYWSWIRQHPGKGLEWIGYIYYSGSTYYNPSLKSRVIISVDTSKNQFSLKLSSVTAADTAVYFCARDYGGNSNYFGYWGQGTLVTVSS

SEQ ID NO: 173 - protein, 128 aa, Homo sapiens
QVQLLQSGAEVKKPGASVKVSCKVSGYTLSELSMHWVRQAPGKGLEWMGGFDPEDGETIYAQKFQGRVTMTEDTSTDTAYMELNSLRSEDTAVYYCATGFAVAGTSEAYYYHYGMDVWGQGTTVTVSS

SEQ ID NO: 174 - protein, 120 aa, Homo sapiens
LVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYSYDNNGWAHWGQGTLVTVSS

SEQ ID NO: 175 - protein, 131 aa, Homo sapiens
EVQLVQSGAEVKKPGESLRISCKGSGYSFTSYWISWVRQMPGKGLEWMGRIDPSDSYTNYSPSFQGLVTISADKSISTAYLQWSSLKASDTAMYYCARGGIRSIAVAYEMTAPLYRGFDPWGQGTLVTVSS

SEQ ID NO: 176 - protein, 122 aa, Homo sapiens
EVQLVESGGGVVQPGRSLRVSCAASGFDFSGFGMHWVRQAPGKGLEWVAIIWYDGSNKNYADSVKGRFTISRDNSKNTLYLQMNSLRAEDTAVYYCAREWHHGSGEGYMDVWGKGTTVTVSS

SEQ ID NO: 177 - protein, 121 aa, Homo sapiens
EVQLLESGGGLVQPGGSLRLSCAASGFTFSSYAMSWVRQAPGKGLEWVSAISGSGGSTYYADSVKGRFTISRDNSKITLYLQVNSLRAEDTAVYYCAKGVVPEAIPWFDPWGQGILVTVSS

SEQ ID NO: 178 - protein, 122 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARVVGSSWPWGFDYWGQGTLVTVSS

SEQ ID NO: 179 - protein, 121 aa, Homo sapiens
QVQLQESGPGLVKPSGTLSLTCAVSNGSVSSINWWSWVRQPPGKGLEWIGEIYHSGSTNYNPSLKSRVTISVDKSKNQISLEMNSVTAADTAFYYCARDRSGGPHGGFDPWGQGTLVTVSS

SEQ ID NO: 180 - protein, 125 aa, Homo sapiens
EVQLVESGGGLVKPGGSLRLSCAASGFSFSDADMSWVRQAPGKGLEWVGRIKTRIAGGTVDYAPPVKGRFTISRDDSKNTLYLQMNSLKTEDTGVYYCTTEGVQSSGWYLSFDFWGQGTLVTVSS

SEQ ID NO: 181 - protein, 128 aa, Homo sapiens
LVQLVQSGAEVKKPGASVKVSCKASGYTFTRYAMHWVRQAPGQRLEWMGWINAGNGNTKYSEKFQGRVTITRDTSASTAYMELSSLRSEDTAVYHCARGLYYYDRIGSQSTEDYFDYWGQGTLVTVSS

SEQ ID NO: 182 - protein, 129 aa, Homo sapiens
QVQLVQSGAEVKKPGASVKVSCKVSGYTLTELSMHWVRQAPGKGLEWMGGFDPEDAETIYAQKFQGRVTMTEDTSTDTAYMELSSLRSEDTAVYYCATSTAVRGERPGGYNYYYGMDVWGQGTTVTVSS

SEQ ID NO: 183 - protein, 127 aa, Homo sapiens
EVQLVQSGAEVKKPGESLRISCKGSGYSFTSYWISWVRQMPGKGLEWMGRIDPSDSYTNYSPSFQGHVSISADKSISTAYLQWSSLKASDTAMYYCARHELVCSGGSCYLEEYFQYWGQGTLVTVSS

SEQ ID NO: 184 - protein, 130 aa, Homo sapiens
QVQLVQSGAEVKKPGASVKVSCKASGYTFTGYYMNWVRQAPGQGLEWMGWINPNSGGTKYIQKFQGRVTMTRDTSISTAYMELSRLRSDDTALYYCARSPVADPWELLRATYYYYGMDVWGQGTTVTVSS

SEQ ID NO: 185 - protein, 129 aa, Homo sapiens
VVQLVQSGAEVKKPGESVRISCKGSGYSFTTYWISWVRQMPGKGLEWMGRIDPSDSYTHYSPSFEGHVTISVDKSVSTAYLQWSSLKASDTAMYYCARQGRWLVPEAEGAWDYNYMDVWGEGTTVTVSS

SEQ ID NO: 186 - protein, 121 aa, Homo sapiens
EVQLVESGGGVVQPGRSLRLSCAASGFTFSTYGMHWVRQAPGKGLEWVAVILYDGSDKYYADSVKGRFTISRDNSKNTLFLQMNSLRAEDTAVYYCVKGGWYGDSLRVDYWGQGTLVTVSS

SEQ ID NO: 187 - protein, 129 aa, Homo sapiens
QVQLQESGPGLVKPSETLSLTCSVSGGSISSNSYYWGWIRQPPGKGLEWIGSMYYSGSTSYNPSLKSRVTISVDTSKNQFSLKLSSVTAADTAVYYCASASKRGYSGYDFISPYYFDYWGQGTLVTVSS

SEQ ID NO: 188 - protein, 124 aa, Homo sapiens
EVQLVESGGGLVKPGGSLRLSCAASGFSFSNAWMTWVRQAPGKGLEWVGRIKTKIDGGATEYAAPVKGRFTISRDDTKNTLYLQMSSLKTEDTAVYYCTTTVSGWFWGDAFDIWGQGTMVTVSS

SEQ ID NO: 189 - protein, 124 aa, Homo sapiens
EVQLVESGGGLVKPGGSLRLSCAASEFTFSNYTMNWVRQAPGKGLEWVSSISSSSSFIYYADSVKGRFTISRDNAKNSLYLQMNSLRAEDTAVYYCARDPSLHRFFRYYYIDVWGKGTTVTVSS

SEQ ID NO: 190 - protein, 128 aa, Homo sapiens
QVQLQESGPGLVKPSETLSLTCTLYGASISSYYWSWIRQPPGKGLEWIGYMYYSGSTNYNPSLKSRVTISVDTSKNQFSLNLSSVTAADTAVYYCARDREGGYCSGGRCYSSHYMDVWGKGTTVTVSS

SEQ ID NO: 191 - protein, 122 aa, Homo sapiens
QVQLVQSGAEVKKPGASMKVSCKASGYTLANYYIHWVRQAPGQGPEWMGIINPSGGATTYAQKFQGRVTMTSDTSTSTVYMELSSLRSEDTAVYYCARGGATPHLRGGMDVWGKGTTVTVSS

SEQ ID NO: 192 - protein, 122 aa, Homo sapiens
EVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARATMAAIRGGWFDPWGQGTLVTVSS

SEQ ID NO: 193 - protein, 131 aa, Homo sapiens
EVQLVESGGGVVQPGRSLRLSCAASGFTFSNFDMHWVRQAPGKGLEWLAIISSDATSKFYADSVKGRITISRDNSKNTLNLLVNSLRAEDTAVYYCARDQGGSYDYVGGSYRLGPTYFDYWGQGTPVTVSS

SEQ ID NO: 194 - protein, 119 aa, Homo sapiens
QVQLQESGPGLVKPSQTLSLTCTVSGGSISSGGYYWSWIRQHPGKGLEWIGYIYSSGSTYYNPSLKSRVTISVDTSKNQFSLKLSSVTAADTAVYYCARVSDFDWFDPWGQGTLVTVSS

SEQ ID NO: 195 - protein, 124 aa, Homo sapiens
EVQLVESGGGVVQPGRSLRLSCAASGFTLSTFGMHWVRQAPGKGLEWVAVISYDGSKKYYADSVKGRFTISRDNSKNTLYLQMNSLRAEDTAVYYCAKDLYYYDTGAPYYFDYWGQGTLVTVSS

SEQ ID NO: 196 - protein, 119 aa, Homo sapiens
EVQLVESGGGLIQPGGSLRLSCAASGFTVSSNYMSWVRQAPGKGLEWVSVIYSGGSTYYADSVKGRFTVSRDNSKNTLYLQMNSLRAEDTAVYYCARGGRYDYDVFDIWGQGTMVTVSS

SEQ ID NO: 197 - protein, 128 aa, Homo sapiens
EVQLVESGGDLVQPGGSLRLSCAASGFTFSSYDMHWVRQATGKGLEWVSTIGTTGDTYYPGSVKGRFTISRENAKNSLYLQMNSLRAEDTAVYYCARGTHIYHHERERGYYYYYMDVWGKGTTVTVSS

SEQ ID NO: 198 - protein, 129 aa, Homo sapiens
EVQLVESGGGVVQPGRSLRLSCAASGFTFSNYGMHWVRQAPGKGLEWVAVISYDGSNKYSADSVKGRFTISRDNSKNTLYLQMNSLRAEDTAVYYCAKVQDPYCSPTNCYTSNYYFDDWGQGTLVTVSS

SEQ ID NO: 199 - protein, 127 aa, Homo sapiens
EVQLVESGGGVVQPGRSLRLSCAASGFTFTNYGMHWVRQAPGKGLEWVAVISYDGSNKYYADSVKGRFTISRDNSKNTLYLQMNSLRTEDTAVYYCTKCGSPYCSGGSCYSCWFDPWGQGTLVIVSS

SEQ ID NO: 200 - protein, 108 aa, Homo sapiens
EIVLTQSPGTLSLSSGERATLSGRASQSVSSSYLAWYQQKPGQAPRLLMYGASSRATGIPDRFSGSGSGTDFTLTISRLEREDFAVYYCQQFRSSPWTFGQGTKVEIK

SEQ ID NO: 201 - protein, 111 aa, Homo sapiens
DIVMTQSPVSLPVTLGEPASISCRSSQSLLHSNGYNYLDWYLQKPGQSPQLLIYLGCNRASGVPDRFSGSGSGTDFTLKISRVEAEDVGVYYCMQALQTPLTLGGGTRVEV

SEQ ID NO: 202 - protein, 109 aa, Homo sapiens
DIQLTQSPSFLSASVGDRVTITCRASQGISSYLAWYQQKPGKAPKLIIYAASTLQSGVPSRFSGSGSGTDFTLTISSLQPEDFATYYCQQLNSYPPGLTFGGGTKVEIK

SEQ ID NO: 203 - protein, 108 aa, Homo sapiens
EIVLTQSPASLSLSPGERATLSCRASQSISSYLAWYQQKPGQVPRLLIYDASSRATGIPARFSGSGSGTDFTLTISSLEPEDFAVYYCQQRSNWPPFTFGPGTKVDIK

SEQ ID NO: 204 - protein, 111 aa, Homo sapiens
QSALTQPPSATGSPGQSVTISCTGTSSDVGGYNHVSWYQQHPGKAPKVMIYEVSKRPSGVPDRFSGSKSGNTASLTVSGLQAEDEADYYCSSYAGRNNFVVFGGGTKLTVL

SEQ ID NO: 205 - protein, 107 aa, Homo sapiens
DIQLTQSPVSLSASVGDRVTITCRASQGISSYLAWYQQKPGKAHKLIIYAASTLQSGVPSRFSGSGSGTEFTLTISSLQPEDFATYYCQQLNSYLYTFGQGTKLEIK

SEQ ID NO: 206 - protein, 108 aa, Homo sapiens
DIQLTQSPSSLSASVGDRVTITCRASQGISSYLAWYQQKPGKAPKLLIYAASTLQSGVPSRFSGSGSGTEFTLTISNLQPEDFATYYCQQLNSYPRFTFGPGTKVDIK

SEQ ID NO: 207 - protein, 112 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVAVYYCMQATQFPYTFGQGTKLEIK

SEQ ID NO: 208 - protein, 110 aa, Homo sapiens
QSALTQPPSASGTPGQRVTISCSGSSSNIGSNFVYWYQQLPGTAPKLLIYRNHLRPSGIPDRFSGSKSGTSASLAISGLRSEDEADYYCAAWDDSLSGWVFGGGTKLTVL

SEQ ID NO: 209 - protein, 107 aa, Homo sapiens
DIQMTQSPFSLSAYVGDRVTITCRASQGIRNDLGWYQQKPGKAPKRLIYVASSFQSGVPSRFSGSGSGTEFTLTISSLQPEDFATYYCLQHNSYPWTFGQGTKVEIK

SEQ ID NO: 210 - protein, 108 aa, Homo sapiens
EIVLTQSPGTLSLSPGERATLSCRASQSVSSSYLAWYQQKPGQAPRLLIYGVSSRATGIPDRFSGSGSGTDFTLTISRLEPEDFAVYYCQQYGSSPRTFGQGTKVEIK

SEQ ID NO: 211 - protein, 106 aa, Homo sapiens
DIQMTQSPSSLSASVGDRVTITCRASQSLSNYLNWYQHKPGKAPKLLIYAASSLQSGVPSRFSGSGSGTDFTLTISSLQPEDFATYYCQQSFSPIFTFGPGTKVDI

SEQ ID NO: 212 - protein, 110 aa, Homo sapiens
SYELTQPPSVSVAPGQTARITCGGNNIGSKSVHWYQQKPGQAPVLVVYDDSDRPSGIPERFSGSNSGNTATLTISRVEAGDEADYYCQVWDSRSDHPGGVFGGGTKLTVL

SEQ ID NO: 213 - protein, 107 aa, Homo sapiens
EIVMTQSPATLSVSPGERATLSCRASQSVRSNLAWYQQKPGQAPRLLIYGASTRATGIPARFSGSGSGTEFTLTISSLQSEDFAVYYCQQYNNWPLTFGGGTKVEIK

SEQ ID NO: 214 - protein, 108 aa, Homo sapiens
DIQLTQSPSSLSASVGDRVTITCRASQGISSYLAWYQQKPGKAPKLLIYAASTLQSGVPSRFSGSGSGTEFTLTISSLQPEDFATYYCQQLNSYPQCTFGPGTKVDIK

SEQ ID NO: 215 - protein, 107 aa, Homo sapiens
DIQMTQSPSSLSASVGDRVTITCQASQDISNYLTWYQQKPGKAPKLLIYDASNLETGVPSRFSGSGSGTDFTFTISSLQPEDIATYYCQQYDNLPLTFGGGTKVEIK

SEQ ID NO: 216 - protein, 109 aa, Homo sapiens
QTVVTQEPSLTVSPGGTVTLTCASSTGAVTSGYYPNWFQQKPGQAPRALIYSTSNKYSWTPARFSGSLLGGKAALTLSGVQPEDEAEYYCLLYYGGARVFGTGTKVTVL

SEQ ID NO: 217 - protein, 109 aa, Homo sapiens
DIQMTQSPSSLSASVGDRVTITCQASQDISNYLNWYQQKPGKAPKLLIYDASNLETGVPSRFSGSGSGTDFTFTISSLQPEDLATYYCQEYDNLPPRRTFGQGTKVEIK

SEQ ID NO: 218 - protein, 112 aa, Homo sapiens
DIVMTQSPVSLPVNIGEPASISCRSSQSLLHSNGYNYLDWYLQKPGQSPQLLISLGCNRASGVPDRFSGSGSGTDFTLKISRVEAEDVGVYSCMQALQTPLTFGGGTKVEIK

SEQ ID NO: 219 - protein, 113 aa, Homo sapiens
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPLTFGPGTKVDIK

SEQ ID NO: 220 - protein, 107 aa, Homo sapiens
EIVLTQSPAPLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 221 - protein, 106 aa, Homo sapiens
EIVMTQSPATLSVSPGERATLSCRASQSISSNLAWYQQKPGQAPRLLIYGASTRATGVPARFSGSGSGTDFTLTISSLQSEDFAVYYCQQYNNWWTFGQGTKVEIK

SEQ ID NO: 222 - protein, 112 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSNTLAVVFGGGTKLTVL

SEQ ID NO: 223 - protein, 107 aa, Homo sapiens
DIQMTQSPFSLSASVGDRVTITCRASQTISNYLNWYQQKPGKAPKLLMYAASTLQSGVPSRFSGSGSGTDFTLTISSLQPEDFATYYCQQSYSTLYTFGQGTKLEIK

SEQ ID NO: 224 - protein, 112 aa, Homo sapiens
QSALTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSAPYVFGTGTKVTVL

SEQ ID NO: 225 - protein, 112 aa, Homo sapiens
DIVMTQTPLSSPVTLGQPASISCRSSQSFVHSDGNTYLSWLQQRPGQPPRLLIYKISGRFCGVPDRFSGSGAGTDFTLKISRVEAEDVGVYYCMQVTQFPHTFGQGTKLEIK

SEQ ID NO: 226 - protein, 110 aa, Homo sapiens
QCVLTRPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGGGTKLTVL

SEQ ID NO: 227 - protein, 107 aa, Homo sapiens
DIQMTQSPSSVYASVGDRVILTCRASQGISRWLAWYQQKPGKARKLLLYAASSLQSGVPSRFSGSGSGTDFTLTINSLQPEDFATYYCLQANSFPYTFGQGTKLEIK

SEQ ID NO: 228 - protein, 108 aa, Homo sapiens
DIQMTQSPSSLSASVGDRVTITCRASQSIGSWLAWYQQKPGKAPKLLIYKASSLESGVPSRFSGSGSGTEFTLTISSLQPDDFATYYCQQYNSYFMFSFGQGTKLEIK

SEQ ID NO: 229 - protein, 108 aa, Homo sapiens
DIQMTQFPFSLFASVGDRVTITCQASQDISNYLNWYQQKPGKAPKLLIYDASNLETGVPSRFSGSGSGTDFTFTISSLQPEDIATYYCQQYDTLPPITFGQGTRLEIK

SEQ ID NO: 230 - protein, 108 aa, Homo sapiens
QSVLTQPPWAYGTPGERFTISCSGSSSNIGSNTVNWYQQLPGTAPKLLIYSNNQRPSGVVDRFSGSKCGTSASLAISGLQSDDEADYYCAAWDDSMIGPVFGGGTKLT

SEQ ID NO: 231 - protein, 107 aa, Homo sapiens
DIQMTQSPSSLSASVGDRVTITCRASQTIGNYVIWYQQKPGKAPKLLIYAASSLQSGVPSRFSGSGSGTDFTLTISSLQPEDLATYYCQQSYNTPRTFGQGTKVEIK

SEQ ID NO: 232 - protein, 111 aa, Homo sapiens
QSALTQPASVSGSPGQSITISCTGTSSDVGSYNLVSWYQQHPGKAPKLMIYEVSKRPSGVSDRFSGSKSGNTASLTISGLQAEDEADYYCCSYAGSSTLKVFGGGTKLTVL

SEQ ID NO: 233 - protein, 108 aa, Homo sapiens
SYELTQPPSVSVAPGKTARITCGENNIGNKSVNWYQQKPGQAPVLVIYYDSDRPSGIPERFSGSNSGNTATLTISWVEAGDEADYYCQVWDSSSDHVVFGGGTKLTDI

SEQ ID NO: 234 - protein, 106 aa, Homo sapiens
DIQMTQSPFSLSASVGDRVTITCRASQSISTWLAWFQQKPGKAPKLLIYKASSLESGVPSRFSGSGSGTEFTLTISSLQPDDFATYYCQQYNSYWTFGQGTKVEIK

SEQ ID NO: 235 - protein, 111 aa, Homo sapiens
DIVMTQTPLSLSVTPGQPASISCKFSQSLLHSDGKTYLYWYLQKPGQPPQLLIYEVSNRFSGVPDRFSGSGSGTDFTVKISRVEAEDVGVYYCMQSIQLWTFGQGTKVEIK

SEQ ID NO: 236 - protein, 108 aa, Homo sapiens
DIQMTQSPSSLSASVGDRVTITCRASQSIGNYLNWYQQKPGKAPKLLIYAASRLQSGVPSRFSGSGSGTDFTLTISSLQPEDFATYYCQQSYSTLALSFGGGTKVEIK

SEQ ID NO: 237 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRFNWPPITFGQGTRLEIK

SEQ ID NO: 238 - protein, 108 aa, Homo sapiens
EIVLTQSPVSLSLSPGERATLSCRASPSVGTYLAWYQQRPGQAPRLLIYDASNRATGIPARFSGSGSGTDFTLTISSLEPEDFAVYYCQQRSNWPLITFGQGTRLEIK

SEQ ID NO: 239 - protein, 108 aa, Homo sapiens
DIQLTPSSFSLSASVGDRVTITCRASQGISSYLAWYQQKPGKAPKLLIYAASTLQSGVPSRFSGSGSGTEFTLTISSLQPEDFATYYCQQLNSYPGLTFGGGTKVEIK

SEQ ID NO: 240 - protein, 108 aa, Homo sapiens
DIQMTQSPSSLSASVGDRVTITCRASQSISSYLNWYQQKPGKAPKLLIYAASSLQSGVPSRFSGSGSGTDFTLTISNLQPADFATYYCQQSYSTPQYTFGQGTKLEIK

SEQ ID NO: 241 - protein, 104 aa, Homo sapiens
DIQLTQSPFSLSASVGDRVTITCRASQGISSYLAWYQQKPGKAPKLLIYAASTLQSGVPSRFSGSGSGTEFTLTISSLQPEDFATYYCQQLGTTFGQGTRLEIK

SEQ ID NO: 242 - protein, 106 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQSDNLPFFGPGTKVDIK

SEQ ID NO: 243 - protein, 123 aa, Homo sapiens
QVQLVQSGPEVKKPGTSVKVSCKASGFNFYYSAVQWVRQARGQGLEWIGWIVVGSGNTNYAQKFQERVTITRDMSTSTAYMELSSLTSEDAAVYYCAAPYCSGGSCHDGFDIWGQGTMVTVSS

SEQ ID NO: 244 - protein, 122 aa, Homo sapiens
QVQLVQSGPEVKKPGTSVKVSCKASGFTFSNSAVQWVRQARGQRLEWIGWIVVGSGNTDYAQKFQERVTITRDMSTSTAYMELSSLRSEDTAVYFCAAPYCGGDCNDGFDVWGQGTMVTVSS

SEQ ID NO: 245 - protein, 122 aa, Homo sapiens
LVQLVQSGAEVKKPGASVKISCKASGYTFTSYYMHWVRQAPGQGLEWMGIINPSGGGTSYAQKFQGRVTMTRDTSTSTVYMELTSLRSDDTAVYYCAKDRVTIFWGNGMDVWGQGTTVTVSS

SEQ ID NO: 246 - protein, 123 aa, Homo sapiens
QVQLVQSGPEVKKPGTSVKVSCKASGFTFSSSAVQWVRQARGQRLEWIGWIVVGSGNANYAPRFQERVTITRDMSTNTAYMELSSLRSEDTAVYYCAAPNCSRTLCYDGFNMWGQGTMVTVSS

SEQ ID NO: 247 - protein, 123 aa, Homo sapiens
QVQLAQSGPEVKKPGTSVKVSCKASGFTFDLSAVQWVRQARGQRLEWIGWIAVGSGNTDYAQKFQERVTLTRDMSTSTAYMELSSLRSEDTAVYYCAAPYCYSTSCADGFDIWGQGTMVTVSS

SEQ ID NO: 248 - protein, 125 aa, Homo sapiens
EVQLVESGGGLVKPGGSLRLSCAASGFTFSNAWMSWVRQAPGKGLEWVGHIKSKTDGGTTDYGAPVKGRFTISRDDSKNTLFLQMNSLKTEDTAVYYCTTDPGWWRIAVAGTNYWGQGTLVTVSS

SEQ ID NO: 249 - protein, 123 aa, Homo sapiens
QVQLVQSGPEVKKPGTSVKVSCKASGFTFTLSAVQWVRLARGQRLEWIGWIVPGSGNVNYAQKFQERVTITRDMSTNTDYMELSSLRSEDTAVYYCAAPYCNKTRCSDGFDIWGQGTMVTVSS

SEQ ID NO: 250 - protein, 123 aa, Homo sapiens
QVQLVQSGPEVKKPGTSVKVSCKASGFTFTSSAVQWVRQARGQRLEWIGWIVVGSGNTNYAQKFQERVTITRDMSTTTAYMEVSSLRSEDTAVYYCAAPNCNRTICADGFDIWGQGTIVTVSS

SEQ ID NO: 251 - protein, 123 aa, Homo sapiens
KIQLVQSGPEVKKPGTSVKVSCKASGFTFTTSAVQWVRQARGQRLEWIGWIAVGSGNTEYEQKFQERVTITRDMSTSTAYMELNSLKSEDTAVYYCAAPYCNTTSCDDGFDIWGQGTMVTVSS

SEQ ID NO: 252 - protein, 124 aa, Homo sapiens
EVQLVESGGGLVQPGGSLRLSCAASGFIFNRYWMTWVRQAPGKGLEWVANIKQDGSEKYYVDSVKGRFTISRDNAKNSLYLQMNSLRAEDTAVYYCAADLGILWFGDLRKSEPWGQGTLVTVSS

SEQ ID NO: 253 - protein, 120 aa, Homo sapiens
QVQLQQWGAGLLKPSETLSLTCVVYGETLSGYYWTWIRQSPGKGLEWIGEISLRGTANYNPSLKSRVTLSVEASKNQFSLKMTSVTAADTAVYYCVGGVVLDNVVWFDPWGQGIPVTVSL

SEQ ID NO: 254 - protein, 123 aa, Homo sapiens
QVQLVQSGPEVKKPGTSVKVSCKASGFTYTTSAVQWVRQARGQRLEWIGWIVAGSGNTNYAQKFQERVTITRDMSTGTAYMELSSLRSEDTAVYYCAAPGCNTTICPDGFDIWGRGTMVTVSS

SEQ ID NO: 255 - protein, 127 aa, Homo sapiens
LVQLVQSGAEVKKPGSSVKVSCKASGGTFSIYAISWVRQAPGQGLEWMGGIIPISGTANYAQKFQDRVTITADESTSTAYMELSSLRSEDTAVYYCARLGRGDYDSSGYYKVYFDYWGQGTLVTVSS

SEQ ID NO: 256 - protein, 123 aa, Homo sapiens
EVQLVQSGVEVRKPGESVRISGKSSGYRFSNYWISWVRQMPGKGLEWMGTIDPSDYYTNYGPSFQGHVTRSGDKSISTAYRQWSSLKASDTAMYYCAKHKFFGELPIRGFDPWGQGTLVTVSS

SEQ ID NO: 257 - protein, 126 aa, Homo sapiens
VVQLVQSGAEVKKPGESLRISCKGSGYSFTTHWISWVRHMPGKGLEWMGRIDPSDSYTTYSPFFQGLVTISVDKSITTAYLHWSSLKASDTAIYYCARENFWSVYYTGIDYYMDVWGKGTTVTVSS

SEQ ID NO: 258 - protein, 120 aa, Homo sapiens
QVQLQQWGAGLLKPSETLSLTCAVYGEPLSGYFWTWIRQPPGKRLEWIGEISLRGSANYNPSLKSRVTISIEVSKNQFSLKLTSVTAADMAVYYCSRGVVLNNVVWFDPWGQGTLVTVSS

SEQ ID NO: 259 - protein, 123 aa, Homo sapiens
EVQLVESGGGVVQPGRSLRLSCAASGFTFSTYAMHWVRQAPGKGLEWVALISYDGSTKYYADSVKGRFTISRDNSKITLYLHMNSLRAEDTAVYYCARDSEDCSSLSCYLDYWGQGTLVTVSS

SEQ ID NO: 260 - protein, 117 aa, Homo sapiens
EVQLVESGGGLVQPGGSLRLSCAASEFIVSRNYMSWVRQAPGKGLEWVSVIYSGGTTYYADSVKGRFTISRDSSKNTLYLQMNSLRAEDTAVYYCARDRGDYLFDYWGQGTLVTVSS

SEQ ID NO: 261 - protein, 122 aa, Homo sapiens
EVQLVESGGGLIQPGGSLRLSCVASGLTVSSNYMNWVRQTPGKGLEWVSVIYSGGSTFYADSVKGRFTISRDNSKNTLFLQMNSLRAEDTAVYYCARVGGYCSSANCVSDVWGKGTTVTVSS

SEQ ID NO: 262 - protein, 117 aa, Homo sapiens
EVQLVESGGGLVQPGGSLRLSCAASGLTVSSNYMSWVRQAPGKGLEWVSVFYPGGSTYNADSVKGRFTISRDNSKNTLYLQMNSLRAEDTAVYYCARDAVYYGMDVWGQGTTVTVSS

SEQ ID NO: 263 - protein, 126 aa, Homo sapiens
VVQLVESGGGLVKPGGSLRLSCAASGFTFSYAWMSWVRQAPGKGLEWVGRIKRKSDGGTTDYAAPVKGRFTISRDDSKNTLYLQMSSLKTEDTAVYYCTTDLCRSTSCEHDAFDIWGQGTMVTVSS

SEQ ID NO: 264 - protein, 116 aa, Homo sapiens
EVQLVESGGGLIQPGGSLRLSCAASGFIVSSNYMSWVRQAPGKGLEWVAVLYPGGSTFYADSVKGRFTISRDNSKNTLYLQMNSLRAEDTAVYYCARNIYDAFDIWGQGTMVTVSS

SEQ ID NO: 265 - protein, 132 aa, Homo sapiens
EVQLVESGGGLVKPGGSLRLSCAASGFTFSDYYMSWIRQAPGKGPEWVSYISGGSSFTNYADSVQGRFTIYRDNAKNSMFLRMNSLRAEDTAVYYCARSPPTGDSSDWYDSPAYNNYYMDVWGKGTTVTVSS

SEQ ID NO: 266 - protein, 115 aa, Homo sapiens
EVQLVESGGGLIQPGGSLRLSCAASGFTVSSNYMSWVRQAPGKGLKWVSVIYSGGSTFYADSVKGRFTISRDDSKNTLFLQMNNLRAEDTAVYYCARLPYGDPAWGQGTLVTVSS

SEQ ID NO: 267 - protein, 123 aa, Homo sapiens
EVQLVESGGGVVQPGRSLRLSCAASGFTFSSFAMHWVRQAPGKGLEWVAVISYDGSTKYSADSVKGRFTISRDNSKNTLYLQMNSLRAEDTAVYYCARDSEDCSSLSCYLDFWGQGTLVTVSS

SEQ ID NO: 268 - protein, 108 aa, Homo sapiens
EIVLTQSPGSLSLSPGERATLSCRASQSVRSNYLAWYQQKPGQAPRVLIYGASSRATGIPDRFSGSGSGTDFTLTISRLEPEDFAVYHCQQYGSSPWTFGQGTKVEIK

SEQ ID NO: 269 - protein, 108 aa, Homo sapiens
EIVLTQSPGSLSLSPGERATLSCRASQSVRSSYLAWYQQKLGQAPGLFIYSTSRRATGIPDRFSGSGSGTDFTLTISRLEPEDFAVYYCQQYGSSPWTFGQGTRVEIK

SEQ ID NO: 270 - protein, 113 aa, Homo sapiens
DIVMTQSPVSLAVSLGERATIKCKSSQSVLYSSNNKNYLGWYQQKPGQPLRLLIYWASTRESGVPDRFSGSGSGTDFTLTISSLQAEDVAVYYCHQYSTTPLTFGGGTKVEIK

SEQ ID NO: 271 - protein, 108 aa, Homo sapiens
EIVLTQSPGSLSLSPGERATLSCRASQSVRSSYLGWYQQKPGQAPRLLIYGASSRATGIPDRFSGSGSETDFTLTISRLEPEDFAVYYCQQYDSSPWTFGQGTKVEIK

SEQ ID NO: 272 - protein, 108 aa, Homo sapiens
EIVLTQFPFSLSLSPGERATLSCRASQSVRSGYLAWYQQKPGQAPRLLIDGTSSRATGIPDRISGSGFGTHFTLTISRVEPEDFAVYYCQQYGSSPWTFGQGTKVEIK

SEQ ID NO: 273 - protein, 107 aa, Homo sapiens
DIQMTQSPFSLSASVGDRVTITCRASQSISSYLNWFQQKPGKAPKLLIYAASSLQSGVPSRFSGSGSGTDFTLTISSLQPEDFATYYCQQSDSSPPTFGQGTKVEFK

SEQ ID NO: 274 - protein, 108 aa, Homo sapiens
EIVLTQSPGSLSLSPGERATLSCRASQSVSSSYLAWYQQKPGQAPRLLIYGASSRASGIPDRFSGSGSGTDFTLTISRLEPEDFAVYYCQQYGSSLFTFGPGTKVDIK

SEQ ID NO: 275 - protein, 108 aa, Homo sapiens
EIGLTQSPCTVSLSPWERTTLSCRASQSVRSSYLAWYQQKPGQAPRLLICATSSRATGIPDRFSGSGSGTDFTLTISRLEPEDFAVYYCQQYGSSPWTFGQGTKVEIK

SEQ ID NO: 276 - protein, 108 aa, Homo sapiens
EIVLTQSPGTLSLSPGERATLSCRASQSVRSNYLAWYQQKPGQAPRVLIYGASSRATGIPDRFSGSGSGTDFTLTISRLEPEDFAVYHCQQYGSSPWTFGQGTKVEIK

SEQ ID NO: 277 - protein, 106 aa, Homo sapiens
DIQMTQSPFSLSASVGDRVSITCRASQGISNSLAWYQQKPGKAPKLLLYAASTLESGVPSRFSGSGSGTDFTLTISSLQPEDFATYYCQEYYSLRTFGQGTKVEIK

SEQ ID NO: 278 - protein, 106 aa, Homo sapiens
ESVLTQSPVSLSLSPGERATLSCRASQSVGTYLAWYQHRPGQAPRLLIYNASKRATGIPARFSGSGSGTDFTLTISSLEPEDLAVYFCQQRSNWLTFGGGTKVEIK

SEQ ID NO: 279 - protein, 108 aa, Homo sapiens
EIVLTQSPGTQSLSPGERATLSCRASQSVSSSYLAWYQQKPGQAPRVLVYGASSRARGIPDRFSGSGSGTDFTLSISRREREDCAVYYCQQYGSLPWTFGQGTKVEIR

SEQ ID NO: 280 - protein, 107 aa, Homo sapiens
DIQMTRSSFSLSASVGDRVTITCRASQNIGNYLNWYQQKPGIAPKLIIYGASSLQSGVPSRFTGSGSGTDFTLTISSMQPEDFATYYCQKSYSGPYTFGQGTKLEIK

SEQ ID NO: 281 - protein, 108 aa, Homo sapiens
EIVLTQSPGSLSLSPGERATLSCRASQSVSGSYIAWYQQKPGQAPRLLISGASNRATGIPDRFSGSGSGTDFTLTISRLEPEDFAVYSCQQYGSSPWTFGQGTKVEIK

SEQ ID NO: 282 - protein, 107 aa, Homo sapiens
DIQMTQSPVSLSASIGDRVTFTCRASQGISNSFAWYQQKPGKAPKLLLYAASRLESGVPSRFSGSGSGTDYTLTISSLQPEDFATYYCQQYYSTPYTFGQGTKIEIK

SEQ ID NO: 283 - protein, 106 aa, Homo sapiens
EIVLTQSPVTLSLSPGERATLSCRASQSVGSYLAWYQQKPGQAPRLLIYGVSNRASGIPARFSGSGSGTDFTLTISGLEPEDFAVYYCQQRSIWLTFGGGTKVEIK

SEQ ID NO: 284 - protein, 113 aa, Homo sapiens
DIVMTQSPDSLAVSLGERATINCKSSQSVLYSSNNKNYLGWYQQKPGQPPKLLIYWASTRESGVPDRFSGSGSGTDFTLTISSLQAEDVAVYYCQQYYSTPFTFGPGTTVDIK

SEQ ID NO: 285 - protein, 107 aa, Homo sapiens
DIQMTQPPSPLFASVGDRVTITCRASQSISSWLAWYQQKPGKAPKLLIYKASSLESGVPSRFSGSGSETEFTLTISSLQPDDFATYYCQQYNSYFPTFGQGTKVEIK

SEQ ID NO: 286 - protein, 107 aa, Homo sapiens
DIQMTQSPSSLSASVGDRVTITCQASQDIRNYLNWYQQKPGKAPKIMIYDASILETGVPSRFSGSGSGTDFTFTISSLQPEDIATYYCQQYDNLPITFGQGTQLEIK

SEQ ID NO: 287 - protein, 109 aa, Homo sapiens
DIQMTQSPSSLSASVGDRVTITCRASQSISSYLNWYQQKPGKAPKLLIYAASSLQSGVPSRFSGSGSGTDFTLTISSLQPEDFAIYYCQESYSTPGLFTFGPGTKVDIK

SEQ ID NO: 288 - protein, 108 aa, Homo sapiens
DIQMTQSPSSLSASVGDRVTVTCRASQSIRSYLNWYQQKPGKAPKLLIYAASSLQSGVPSRFSGSGSGTDFTLTISSLQPEDFATYYCQQSYTTPAITFGQGTRVQIK

SEQ ID NO: 289 - protein, 108 aa, Homo sapiens
DIQLTQSPSSLSASVGDRVTITCRASQGIGSYLAWYQQKPGKAPKLLIYAASTLQSGVPSRFSGSGSGTEFTLTISSLQPEDFATYYCQQLNSYPQGAFGQGTKVDIK

SEQ ID NO: 290 - protein, 109 aa, Homo sapiens
EIVLTQSPVTLSLSPGERATLSCRASQSVSSSYLAWYQQKPGQAPRLLIYGASSRATGIPDRFSGSGSGTDFTLTISRLEPEDFAVYYCQQYGTSPTVTFGQGTRLEIK

SEQ ID NO: 291 - protein, 107 aa, Homo sapiens
DIQMTQSPSSLSASVGDRVTITCLATQDIRNYLHWYQQKPGKAPKLIIYDASHLETGVPSRFSGSGSGTDFTFTISSLQPEDIATYYCLQYDNLPLTFGGGTKLEIK

SEQ ID NO: 292 - protein, 113 aa, Homo sapiens
DIVMTQSPVSLAVSLGERATINCKSSQSVLYRSNNKNYLAWYQQKPGQPPKLLIYWASTRGSGVPDRFSGSGSGTDFTLTISSLQAEDVAVYYCQQYYSTPFTFGPGTKVDVK